(19) United States
(12) Patent Application Publication
(10) Pub. No.: US 2026/0224589 A1
(43) Pub. Date: Aug. 6, 2026

(54) KRAS INHIBITORS

(71) Applicant: Erasca, Inc., San Diego, CA (US)

(72) Inventors: Nicholas A. Isley, San Diego, CA (US); Jun Feng, San Diego, CA (US); Jean-Michel Vernier, San Diego, CA (US); Marcos Gonzalez-Lopez, San Diego, CA (US); Peter C. Chua, San Diego, CA (US); Ping Chen, San Diego, CA (US); Adriana Irimia, San Diego, CA (US); Hengmiao Cheng, San Diego, CA (US); Takasuke Mukaiyama, San Diego, CA (US); Lucas Nguyen, San Diego, CA (US)

(21) Appl. No.: 19/157,251

(22) PCT Filed: Feb. 16, 2024
(86) PCT No.: PCT/US2024/016228

§ 371 Date: Aug. 15, 2025

Related U.S. Application Data
(60) Provisional application No. 63/485,723, filed on Feb. 17, 2023.
(60) Provisional application No. 63/593,377, filed on Oct. 26, 2023.
(60) Provisional application No. 63/600,469, filed on Nov. 17, 2023.
(60) Provisional application No. 63/615,162, filed on Dec. 27, 2023.

(51) Int. Cl. A61K 31/553; A61K 31/519; A61K 31/551; A61K 31/554; C07B 59/00; C07D 519/00

(52) U.S. Cl. CPC A61K 31/553; A61K 31/519; A61K 31/551; A61K 31/554; C07B 59/002; C07D 519/00; C07B 2200/05

(57) ABSTRACT

The present embodiments provide compounds of Formula (I), compositions of the compounds, and methods for treating diseases such as cancer.

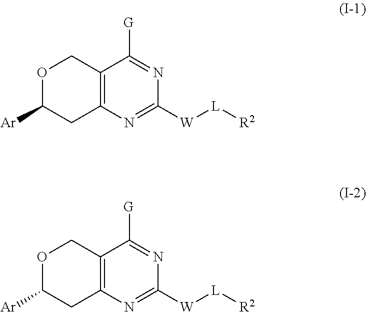

### BACKGROUND

Embodiments herein relate to compounds, compositions and methods for the treatment of RAS-mediated disease. In particular, embodiments herein relate to compounds and methods for treating diseases such as cancer via targeting oncogenic mutants of the K-RAS isoform.

Ras proteins are small guanine nucleotide-binding proteins that act as molecular switches by cycling between active GTP-bound and inactive GDP-bound conformations. Ras signaling is regulated through a balance between activation by guanine nucleotide exchange factors (GEFs), most commonly son of sevenless (SOS), and inactivation by GTPase-activating proteins (GAPs) such as neurofibromin or p120GAP. The Ras proteins play an important role in the regulation of cell proliferation, differentiation, and survival. Dysregulation of the Ras signaling pathway is almost invariably associated with disease. Hyper-activating somatic mutations in Ras are among the most common lesions found in human cancer. Most of these mutations have been shown to decrease the sensitivity of Ras to GAP stimulation and decrease its intrinsic GTPase activity, leading to an increase in the active GTP-bound population. Although mutation of any one of the three Ras isoforms (K-Ras, N-Ras, or H-Ras) has been shown to lead to oncogenic transformation, K-Ras mutations are by far the most common in human cancer. For example, K-Ras mutations are known to be often associated with pancreatic, colorectal and non-small-cell lung carcinomas. Similarly, H-Ras mutations are common in cancers such as papillary thyroid cancer, lung cancers and skin cancers. Finally, N-Ras mutations occur frequently in hepatocellular carcinoma.

K-Ras is the most frequently mutated oncoprotein in human cancers, and the G12D mutation is among the most prevalent. Accordingly, there is a need to develop selective inhibitors of KRAS G12D or G12V. The present embodiments meet this and other needs.

### SUMMARY

In one aspect, the present embodiments provide compounds of Formula (I-1) or Formula (I-2):

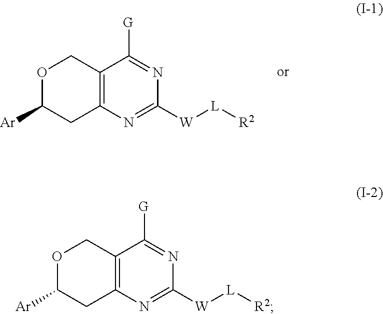

or a stereoisomer, a tautomer, or a pharmaceutically acceptable salt thereof, wherein:
   G is

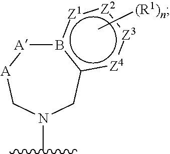

Ar is aryl or heteroaryl;
   wherein Ar is optionally substituted with 1 to 5 groups selected from —OH, halo, C₁₋₃ alkyl, C₂-C₄ alkynyl, C₁₋₃ haloalkyl, C₃₋₆ cycloalkyl, a fused C₃₋₆ cycloalkyl, —CN, —CD₃, —SF₅ and —NH₂;
A is —CF₂, —CR^{7a}R^{7b}, or bond;
A′ is —CH₂—, —O—, —NH—, —S—, and —C═O;
B is C or N;
W is —O—, —NR³, —S—,

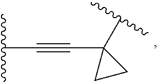 [SMILES: *C#CC1(*)CC1]

or absent;
L is C₁₋₃ alkyl or absent;
each of Z¹, Z², Z³, Z⁴ is independently null, CR¹, CH, O, N(R¹), NH, N, or S;
   wherein Z¹, Z², Z³, Z⁴ combine to form a 5- or 6-membered aromatic or heteroaromatic ring;
each R¹ is an optional substitution and is independently halo, C₁₋₆ alkyl, —OC₁-C₆, —(CH₂)ₙ—N(R^{1b})(R^{1c}), —C(O)R^{1a}, —C(O)—N(R^{1b})(R^{1c}), —C(═NH)N(R^{1b})(R^{1c}), —C(═S)N(R^{1b})(R^{1c}), —NH—(CH₂)ₙ—(SO)ₚR^{1a}, —S(O)ₚR^{1a}, —S(O)ₚ(R^{1b})(R^{1c}), —S(O)ₚN(R^{1b})(R^{1c}), —P(O)(R^{1b})(R^{1c}), C₃₋₆ cycloalkyl, 5- or 6-membered heteroaromatic ring or

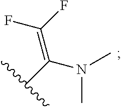 [SMILES: *C(=C(F)F)N(C)C]

or
   two adjacent R¹ combine to form a 5- to 7-membered aryl, 5- to 7-membered heteroaryl, 3- to 6-membered carbocylic, or 4- to 6-membered heterocyclic ring, any of which are optionally substituted with OH, amino, —C(O)N(R^{1b})(R^{1c}), halo, CN, CF₃, C₁₋₄ alkyl, C₁₋₄ alkoxy, and C₁₋₃ hydroxyalkyl; wherein the 5- to 7-membered heteroaryl or 4- to 6-membered heterocyclic ring comprises carbon atoms and 1-4 groups selected from halo, —O—, C═O, —S(O)ₚ, —S(O)ₚNH—, —NH—, and —N(C₁₋₄ alkyl);
   R^{1a} is selected from halo, C₁₋₆ alkyl, C₁₋₃ hydroxyalkyl, C₁₋₃ haloalkyl, and 4- to 6-membered heterocyclyl;
   R^{1b} and R^{1c} are each independently selected from hydrogen, CD₃, C₁₋₆ alkyl, —OC₁-C₆, —(CH₂)ₙOH, —(CH₂)ₙOR^{1a}, —(CH₂)ₙCN, C₁₋₃ haloalkyl and 3- to 6-membered carbocyclic optionally substituted with halo; or
   R^{1b} and R^{1c} combine to form a 5- to 7-membered aryl, 5- to 7-membered heteroaryl, 3- to 6-membered carbocylic, or 4- to 10-membered heterocyclic ring, wherein the 5- to 7-membered heteroaryl or 4- to 10-membered heterocyclic ring comprises carbon atoms and 1-4 groups selected from —O—, C═O, S(O)ₚ, S(O)ₚNH, —NH—, and —N(C₁₋₄ alkyl), wherein the 5-to 7-membered aryl, 5- to 7-membered heteroaryl, 3- to 6-membered carbocylic, or 4- to 10-membered heterocyclic rings are optionally substituted with halo, —OH, C₁₋₄ alkyl, C₁₋₃ haloalkyl, C₁₋₄ alkoxy, —(CH₂)ₙN(R^{1d})(R^{1e}), C₁₋₄ hydroxyalkyl, —(CH₂)ₙOR^{1a}, —C(O)N(R^{1d})(R^{1e});
   R^{1d} and R^{1e} are each independently selected from hydrogen, CD₃, and C₁₋₆ alkyl;
   wherein when R¹ is aryl or heteroaryl, R¹ is optionally substituted with —OH, halo, C₁₋₃ alkyl, C₂-C₄ alkynyl, C₁₋₃ haloalkyl, —CN, —NH₂, or combinations thereof;
R² is a 4- to 10-membered heterocyclyl, 3- to 10-membered cycloalkyl, 6- to 10-membered aryl or 5- to 10-membered heteroaryl, wherein said heterocyclyl, cycloalkyl, aryl and heteroaryl are optionally substituted with deuterium, OH, halo, CN, CF₃, C₁₋₄ alkyl, C₁₋₄ alkoxy, C₂₋₄ alkenyl, C₁₋₄ haloalkenyl, —N(R^{1d})(R^{1e}), oxo, and C₁₋₃ hydroxyalkyl;
R^{7a} and R^{7b} are independently selected from hydrogen and C₁₋₄ alkoxy, or R^{7a} and R^{7a} may optionally be taken together with the atom to which they are attached to form a 4- to 6-membered spirocyclic ring wherein any one of the carbon atoms may be replaced by —O—, —S—, or —NH—;
n, at each occurrence, is an integer independently selected from 0, 1, 2, 3, and 4; and,
p, at each occurrence, is an integer independently selected from 0, 1, and 2.

In another aspect, the present embodiments provide compounds of Formula (I-1):

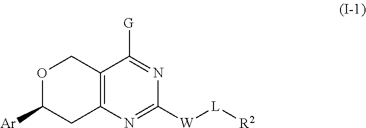

or a stereoisomer, a tautomer, a pharmaceutically acceptable salt thereof;
wherein G is

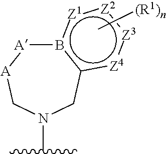

Ar is aryl or heteroaryl;
   wherein Ar is optionally substituted with —OH, halo, C₁₋₃ alkyl, C₂-C₄ alkynyl, C₁₋₃ haloalkyl, —CN, CF₃, —NH₂, or combinations thereof;
A is —CH₂, —CF₂, or bond;
A′ is —CH₂, O, NH, O, S, and —C═O;
B is C or N;
each of Z¹, Z², Z³, Z⁴ is independently null, CR¹, O, N(R¹), NH, or S;
   wherein of Z¹, Z², Z³, Z⁴ combine to form a 5- or 6-membered aromatic ring; each is independently CH or N;
W is O, NR³, S, or absent;
L is C₁₋₃ alkyl or absent;
each R¹ is independently N-alkylamido, N,N-dialkylamido, halo, C₁₋₆ alkyl or C₃₋₆ cycloalkyl, aryl, or heteroaryl, wherein when R¹ is aryl or heteroaryl, R¹ is optionally substituted with —OH, halo, C₁₋₃ alkyl, C₂-C₄ alkynyl, C₁₋₃ haloalkyl, —CN, CF₃, —NH₂, or combinations thereof; or
   two R¹ combine to form a 5- to 7-membered aryl, heteroaryl, carbocylic, or heterocyclic ring, any of which are optionally substituted with OH, amino, N-alkylamido, N,N-dialkylamido, halo, CN, CF₃, C₁₋₄ alkyl, C₁₋₄ alkoxy, and C₁₋₃ alkyl-OH;
   R² is 4-10 membered heterocyclyl, 3-10 membered cycloalkyl, 6-10 membered aryl or 5-10 membered heteroaryl, wherein said heterocyclyl, cycloalkyl, aryl and heteroaryl are optionally substituted with deuterium, OH, halo, CN, CF₃, C₁₋₄ alkyl, C₁₋₄ alkoxy, and C₁₋₃ alkyl-OH;
and,
   n is 0, 1, 2, 3, or 4.

In another aspect, the present embodiments provide a pharmaceutical composition comprising a pharmaceutically effective amount of the compounds disclosed herein, or a pharmaceutically acceptable salt thereof, and a pharmaceutically acceptable excipient.

In another embodiment, the present embodiments provide a method of treating a subject having cancer, the cancer characterized by the presence of a KRAS G12D or G12V mutation, the method comprising administering to the subject a therapeutically effective amount of a compound disclosed herein, or a pharmaceutically acceptable salt thereof, or a pharmaceutical composition as disclosed herein.

In another embodiment, the present embodiments provide a method for manufacturing a medicament for treating a subject having cancer, the cancer characterized by the presence of a KRAS G12D or G12V mutation, the medicament comprising a compound disclosed herein, or a pharmaceutically acceptable salt thereof, or a a pharmaceutical composition as disclosed herein, is used.

In another embodiment, the present embodiments provide for the use of a compound disclosed herein, or a pharmaceutically acceptable salt thereof, or a a pharmaceutical composition as disclosed herein, for the manufacture of a medicament for the treatment of cancer in a subject, the cancer characterized by the presence of a KRAS G12D or G12V mutation.

In another embodiment, the present embodiments provide the compounds disclosed herein, or a pharmaceutically acceptable salt thereof, or a a pharmaceutical composition as disclosed herein, for use in the treatment of cancer in a subject, the cancer characterized by a KRAS G12D or G12V mutation.

### DETAILED DESCRIPTION

### I. General

The present embodiments provide inhibitors of KRAS G12D or G12V and KRAS G12V and are useful for treating a cancer characterized by a KRAS G12D or G12V and G12V mutations.

### II. Definitions

Unless specifically indicated otherwise, all technical and scientific terms used herein have the same meaning as commonly understood by those of ordinary skill in the art to which the embodiments belong. In addition, any method or material similar or equivalent to a method or material described herein can be used in the practice of the present embodiments. For purposes of the present embodiments, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂-s, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted. Alkenyl can include a C₁ fragment, or longer, in which the carbon point of attachment is part of the unsaturation in the alkeynl group, i.e., alkenyl includes carbenyl fragments (the resultant pendant group being an alkylidene). Accordingly, alkenyl includes alkylidene substitutions. An example of alkylidene is ═C(R′)(R″). In a more specific example, the alkylidene substitution is

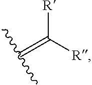

where R′ and R″ represent substitution at the vinylic position.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene. Alkenylene groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, isopropynylene, butynylene, sec-butynylene, pentynylene and hexynylene. Alkynylene groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The alkoxy component is as defined above. Examples of the alkoxyalkyl group include, but are not limited to, 2-ethoxy-ethyl and methoxymethyl.

“Alkylhydroxy” or “hydroxyalkyl” refers to an alkyl group, as defined above, where at least one of the hydrogen atoms is replaced with a hydroxy group. As for the alkyl group, alkylhydroxy groups can have any suitable number of carbon atoms, such as C₁₋₆. Exemplary alkylhydroxy groups include, but are not limited to, hydroxy-methyl (e.g., —CH₂OH), hydroxyethyl (where the hydroxy is in the 1- or 2-position) (e.g., —CHOHCH₃ or —CH₂CH₂OH), hydroxypropyl (where the hydroxy is in the 1-, 2- or 3-position) (e.g., —CHOHCH₂CH₃, —CH₂CHOHCH₃, or —CH₂CH₂CH₂OH), hydroxybutyl (where the hydroxy is in the 1-, 2-, 3- or 4-position) (e.g., —CHOHCH₂CH₂CH₃, —CH₂CHOHCH₂CH₃, —CH₂CH₂CHOHCH₃, or —CH₂CH₂CH₂CH₂OH), hydroxypentyl (where the hydroxy is in the 1-, 2-, 3-, 4- or 5-position) (e.g., —CHOHCH₂CH₂CH₂CH₃, —CH₂CHOHCH₂CH₂CH₃, —CH₂CH₂CHOHCH₂CH₃, —CH₂CH₂CH₂CH₂CHOHCH₃, or —CH₂CH₂CH₂CH₂CH₂OH), hydroxyhexyl (where the hydroxy is in the 1-, 2-, 3-, 4-, 5- or 6-position), 1,2-dihydroxyethyl, and the like.

“Halogen” or “halo” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl. Similarly, “haloalkenyl” refers to an alkenyl, as defined above, where some or all of the hydrogen atoms are replaced with halogen atoms. Haloalkenyl includes alkylidene substitutions. having one or more halogen substitutions. An example of alkylidene is ═C(R′)(R″). In a more specific example, the alkylidene substitution is

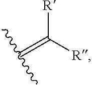

where R′ and R″ represent substitution at the vinylic position. A haloalkenyl, accordingly, refers to an alkylidene substitution where either R′ or R″ or both R′ and R″ include a halogen.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2-trifluoroethoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Cycloalkylene” refers to a cycloalkyl group having the number of carbon atoms indicated, and linking at least two other groups, i.e., a divalent radical. The two moieties linked to the cycloalkylene can be linked to the same atom or different atoms of the cycloalkylene group. Examples of cycloalkylene rings include cyclopropylene, cyclobutylene, cyclopentylene and cyclohexylene, among others. Cycloalkylene groups can be linked 1,1, 1,2, 1,3, or 1,4. The cyclohexylene ring, for example, can adopt a number of conformations, including the boat and chair conformations. The chair conformation of cyclohexylene can have substituents in an axial or equatorial orientation. The divalent nature of the cycloalkylenes results in cis and trans formations where cis refers to both substituents being on the same side (top or bottom) of the cycloalkylene ring, and where trans refers to the substituents being on on opposite sides of the cycloalkylene ring. For example, cis-1,2- and cis-1,4-cyclohexylene can have one substituent in the axial orientation and the other substituent in the equatorial orientation, while trans-1,2- and trans-1,4-cyclohexylene have both substituents in the axial or equatorial orientation. cis-1,3-cyclohexylene have both substituents in the axial or equatorial orientation, and trans-1,3-cyclohexylene can have one substituent in the axial orientation and the other substituent in the equatorial orientation. Cycloalkylene groups can be substituted or unsubstituted.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” or “heterocyclyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, dithiane, and hexahydro-1H-pyrrolizine. The heterocycloalkyl groups can also be fused to aromatic or non-aromatic ring systems to form members including, but not limited to, indoline. The heterocycloalkyl group include both spirocyclic and/or bridged heterocyclyl groups. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, morpholine, and hexahydro-1H-pyrrolizine.

“Heterocyclalkylene” refers to a heterocyclalkyl group, as defined above, linking at least two other groups. The two moieties linked to the heterocyclalkylene can be linked to the same atom or different atoms of the heterocyclalkylene. Heterocycloalkylene groups can be substituted or unsubstituted.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above. Alkyl-heterocycloalkyl groups can be substituted or unsubstituted.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Arylene” refers to an aryl group, as defined above, linking at least two other groups. The two moieties linked to the aryl can be linked to the same atom or different atoms of the aryl. Arylene groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroarylene” refers to a heteroaryl group, as defined above, linking at least two other groups. The two moieties linked to the heteroaryl are linked to different atoms of the heteroaryl. Heteroarylene groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

The groups defined above can optionally be substituted by any suitable number and type of substituents. Representative substituents include, but are not limited to, halogen, haloalkyl, haloalkoxy, —OR′, ═O, —OC(O)R′, —(O)R′, —O₂R′, —ONR′R″, —OC(O)NR′R″, =NR′, =N—OR′, —NR′R″, —NR″C(O)R′, —NR′—(O)NR″R′″, —NR″C(O)OR′, —NH—(NH₂)=NH, —NR′C(NH₂)=NH, —NH—(NH₂)═NR′, —SR′, —S(O)R′, —S(O)₂R′, —S(O)₂NR′R″, —NR′S(O)₂R″, —N₃ and —NO₂. R′, R″ and R′″ each independently refer to hydrogen, unsubstituted alkyl, such as unsubstituted C₁₋₆ alkyl. Alternatively, R′ and R″, or R″ and R′″, when attached to the same nitrogen, are combined with the nitrogen to which they are attached to form a heterocycloalkyl or heteroaryl ring, as defined above.

“Salt” refers to acid or base salts of the compounds, which can be used in the methods disclosed herein. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds disclosed herein are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present embodiments.

Certain compounds disclosed herein possess asymmetric carbon atoms (optical centers) or double bonds; the racemates, diastereomers, geometric isomers and individual isomers are all intended to be encompassed within the scope of the present embodiments.

“Hydrate” refers to a compound that is complexed to at least one water molecule. The compounds disclosed herein can be complexed with from 1 to 10 water molecules.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, coatings, sweeteners, flavors and colors. One of skill in the art will recognize that other pharmaceutical excipients are useful in the present embodiments.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Subject” refers to animals such as mammals, including, but not limited to, primates (e.g., humans), cows, sheep, goats, horses, dogs, cats, rabbits, rats, mice and the like. In certain embodiments, the subject is a human.

### III. Compounds

The present embodiments provide compounds of Formula (I-1) or Formula (I-2):

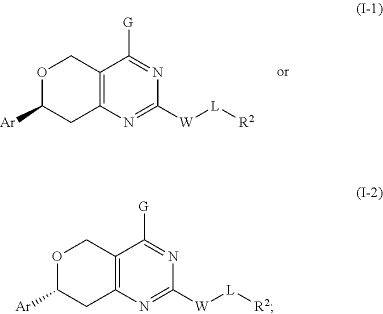

or a stereoisomer, a tautomer, or a pharmaceutically acceptable salt thereof, wherein:
   G is

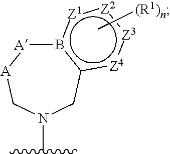

Ar is aryl or heteroaryl;
   wherein Ar is optionally substituted with 1 to 5 groups selected from —OH, halo, C₁₋₃ alkyl, C₂-C₄ alkynyl, C₁₋₃ haloalkyl, C₃₋₆ cycloalkyl, a fused C₃₋₆ cycloalkyl, —CN, —CD₃, —SF₅ and —NH₂;
A is —CF₂, —CR^{7a}R^{7b}, or bond;
A′ is —CH₂—, —O—, —NH—, —S—, and —C═O;
B is C or N;
W is —O—, —NR³, —S—,

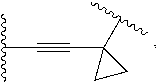 [SMILES: *C#CC1(*)CC1]

or absent;
L is C₁₋₃ alkyl or absent;
each of Z¹, Z², Z³, Z⁴ is independently null, CR¹, CH, O, N(R¹), NH, N, or S;
   wherein Z¹, Z², Z³, Z⁴ combine to form a 5- or 6-membered aromatic or heteroaromatic ring;
each R¹ is an optional substitution and is independently halo, C₁₋₆ alkyl, —OC₁-C₆, —(CH₂)ₙ—N(R^{1b})(R^{1c}), —C(O)R^{1a}, —C(O)—N(R^{1b})(R^{1c}), —C(═NH)N(R^{1b})(R^{1c}), —C(═S)N(R^{1b})(R^{1c}), —NH—(CH₂)ₙ—(SO)ₚR^{1a}, —S(O)ₚR^{1a}, —S(O)ₚ(R^{lb})(R^{1c}), —S(O)ₚN(R^{1b})(R^{1c}), —P(O)(R^{1b})(R^{1c}), C₃₋₆ cycloalkyl, 5- or 6-membered heteroaromatic ring or

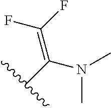 [SMILES: *C(=C(F)F)N(C)C]

or
   two adjacent R¹ combine to form a 5- to 7-membered aryl, 5- to 7-membered heteroaryl, 3- to 6-membered carbocylic, or 4- to 6-membered heterocyclic ring, any of which are optionally substituted with OH, amino, —C(O)N(R^{1b})(R^{1c}), halo, CN, CF₃, C₁₋₄ alkyl, C₁₋₄ alkoxy, and C₁₋₃ hydroxyalkyl; wherein the 5- to 7-membered heteroaryl or 4- to 6-membered heterocyclic ring comprises carbon atoms and 1-4 groups selected from halo, —O—, C═O, —S(O)ₚ, —S(O)ₚNH—, —NH—, and —N(C₁₋₄ alkyl);
   R^{1a} is selected from halo, C₁₋₆ alkyl, C₁₋₃ hydroxyalkyl, C₁₋₃ haloalkyl, and 4- to 6-membered heterocyclyl;
   R^{1b} and R^{1c} are each independently selected from hydrogen, CD₃, C₁₋₆ alkyl, —(CH₂)ₙOH, —(CH₂)ₙOR^{1a}, —(CH₂)ₙCN, C₁₋₃ haloalkyl and 3- to 6-membered carbocyclic optionally substituted with halo; or
   R^{1b} and R^{1c} combine to form a 5- to 7-membered aryl, 5- to 7-membered heteroaryl, 3- to 6-membered carbocylic, or 4- to 10-membered heterocyclic ring, wherein the 5- to 7-membered heteroaryl or 4- to 10-membered heterocyclic ring comprises carbon atoms and 1-4 groups selected from —O—, C═O, S(O)ₚ, S(O)ₚNH, —NH—, and —N(C₁₋₄ alkyl), wherein the 5-to 7-membered aryl, 5- to 7-membered heteroaryl, 3- to 6-membered carbocylic, or 4- to 10-membered heterocyclic rings are optionally substituted with halo, —OH, C₁₋₄ alkyl, C₁₋₃ haloalkyl, C₁₋₄ alkoxy, —(CH₂)ₙN(R^{1d})(R^{1e}), C₁₋₄ hydroxyalkyl, —(CH₂)ₙOR^{1a}, —C(O)N(R^{1d})(R^{1c});
   R^{1d} and R^{1c} are each independently selected from hydrogen, CD₃, and C₁₋₆ alkyl;
   wherein when R¹ is aryl or heteroaryl, R¹ is optionally substituted with —OH, halo, C₁₋₃ alkyl, C₂-C₄ alkynyl, C₁₋₃ haloalkyl, —CN, —NH₂, or combinations thereof;
R² is 4- to 10-membered heterocyclyl, 3- to 10-membered cycloalkyl, 6- to 10-membered aryl or 5- to 10-membered heteroaryl, wherein said heteroocyclyl, cycloalkyl, aryl and heteroaryl are optionally substituted with deuterium, OH, halo, CN, CF₃, C₁₋₄ alkyl, C₁₋₄ alkoxy, C₂₋₄ alkenyl, C₁₋₄ haloalkenyl, —N(R^{1d})(R^{1e}), oxo, and C₁₋₃ hydroxyalkyl;
R^{7a} and R^{7b} are independently selected from hydrogen and C₁₋₄ alkoxy, or R^{7a} and R^{7a} may optionally be taken together with the atom to which they are attached to form a 4- to 6-membered spirocyclic ring wherein any one of the carbon atoms may be replaced by —O—, —S—, or —NH—;
n, at each occurrence, is an integer independently selected from 0, 1, 2, 3, and 4; and,
p, at each occurrence, is an integer independently selected from 0, 1, and 2.

In embodiments, the present embodiments provide compounds, and pharmaceutically acceptable salts thereof, of Formula (I-1):

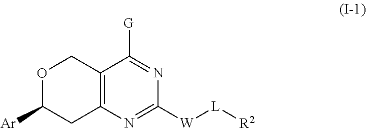

or a stereoisomer, a tautomer, a pharmaceutically acceptable salt thereof;
wherein G is

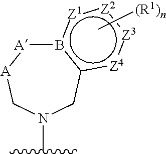

Ar is aryl or heteroaryl;
   wherein Ar is optionally substituted with —OH, halo, C₁₋₃ alkyl, C₂-C₄ alkynyl, C₁₋₃ haloalkyl, —CN, CF₃, —NH₂, or combinations thereof;
A is —CH₂, —CF₂, or bond;
A′ is —CH₂, O, NH, O, S, and —C═O;
B is C or N;
each of Z¹, Z², Z³, Z⁴ is independently null, CR¹, O, N(R¹), NH, or S;
   wherein of Z¹, Z², Z³, Z⁴ combine to form a 5- or 6-membered aromatic ring;
each is independently CH or N;
W is O, NR³, S, or absent;
L is C₁₋₃ alkyl or absent;
each R¹ is independently N-alkylamido, N,N-dialkylamido, halo, C₁₋₆ alkyl or C₃₋₆ cycloalkyl, aryl, or heteroaryl, wherein when R¹ is aryl or heteroaryl, R¹ is optionally substituted with —OH, halo, C₁₋₃ alkyl, C₂-C₄ alkynyl, C₁₋₃ haloalkyl, —CN, CF₃, —NH₂, or combinations thereof; or
   two R¹ combine to form a 5- to 7-membered aryl, heteroaryl, carbocylic, or heterocyclic ring, any of which are optionally substituted with OH, amino, N-alkylamido, N,N-dialkylamido, halo, CN, CF₃, C₁₋₄ alkyl, C₁₋₄ alkoxy, and C₁₋₃ alkyl-OH;
R² is 4-10 membered heterocyclyl, 3-10 membered cycloalkyl, 6-10 membered aryl or 5-10 membered heteroaryl, wherein said heterocyclyl, cycloalkyl, aryl and heteroaryl are optionally substituted with deuterium, OH, halo, CN, CF₃, C₁₋₄ alkyl, C₁₋₄ alkoxy, and C₁₋₃ alkyl-OH;
and,
n is 0, 1, 2, 3, or 4.

In some embodiments, the compound is a single atropisomer of Formula (I-1) or Formula (I-2).

In some embodiments, the compound is of Formula (Ia):

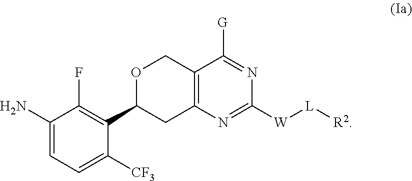

In some embodiments, the compound is of Formula (Ib):

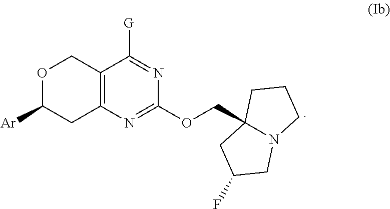

In some embodiments, the compound is of Formula (Ic):

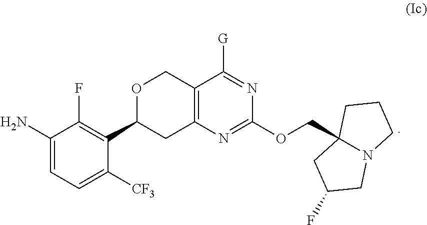

In some embodiments, the compound is of Formula (Id):

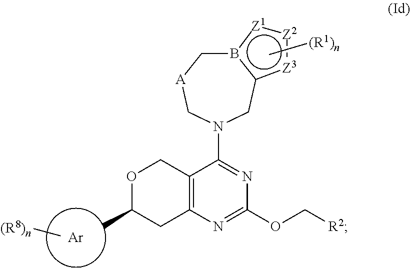

or a stereoisomer, a tautomer, or a pharmaceutically acceptable salt thereof,
wherein:
Ar is phenyl or pyridine;
each R⁸ is independently selected from NH₂, OH, CH₃, F, Cl, Br, I, CF₃ and CD₃;
Z¹ is O or N,
Z² is CR¹, CH, O, N(R¹), NH, or N;
Z³ is CR¹, CH, O, N(R¹), NH, or S; and,
n, at each occurrence, is an integer independently selected from 2, 3 and 4.

In some embodiments, Ar is:

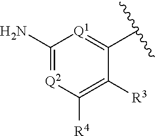

wherein Q¹ and Q² are independently CR⁷ or N; and,
each R³, R⁴, R⁵ and R⁷ are independently selected from H, CH₃, F, Cl, Br, I, CF₃ and CD₃.

In some embodiments, Ar is:

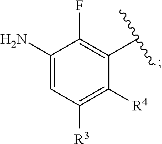

wherein R³ is H, Me, CF₃, Br, Cl, or CD₃; and
R⁴ is H, Me, CF₃, Cl, Br, or I.

In some embodiments, Ar is:

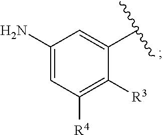

wherein R³ is H, Me, CF₃, Br, Cl, or CD₃; and
R⁴ is H, Me, CF₃, Cl, Br, or I

In some embodiments, Ar is selected from:

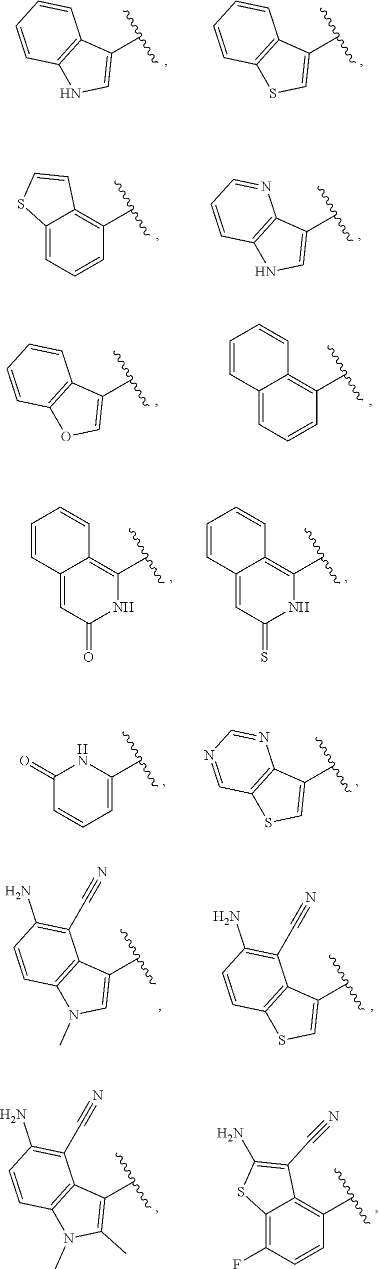

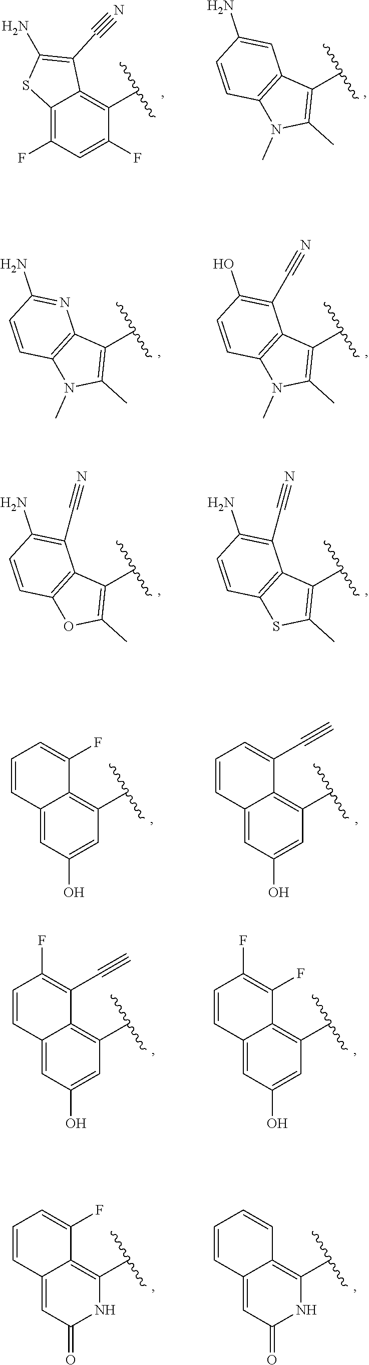

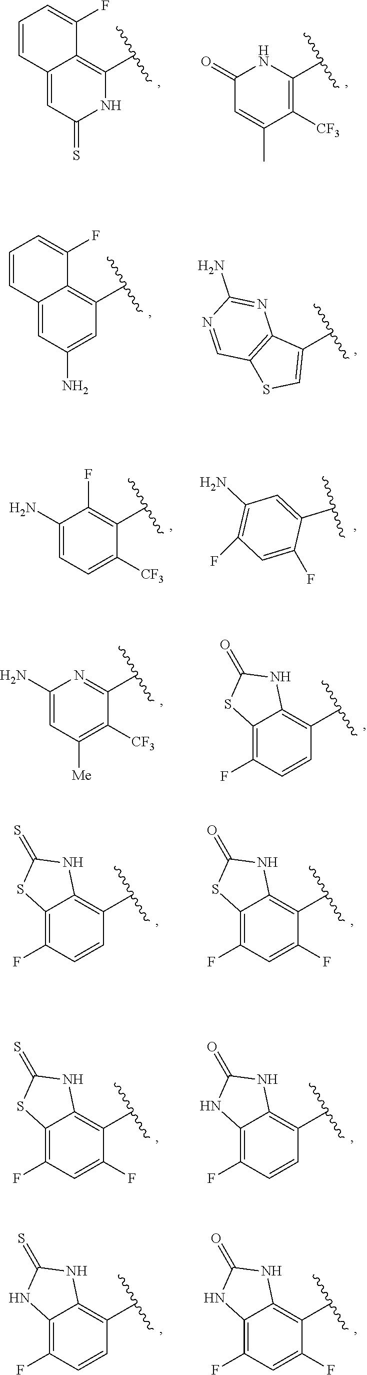

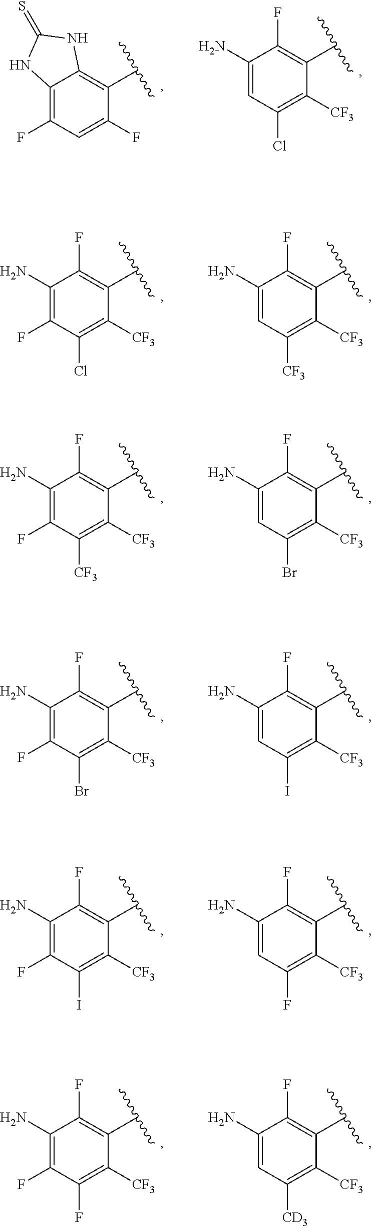

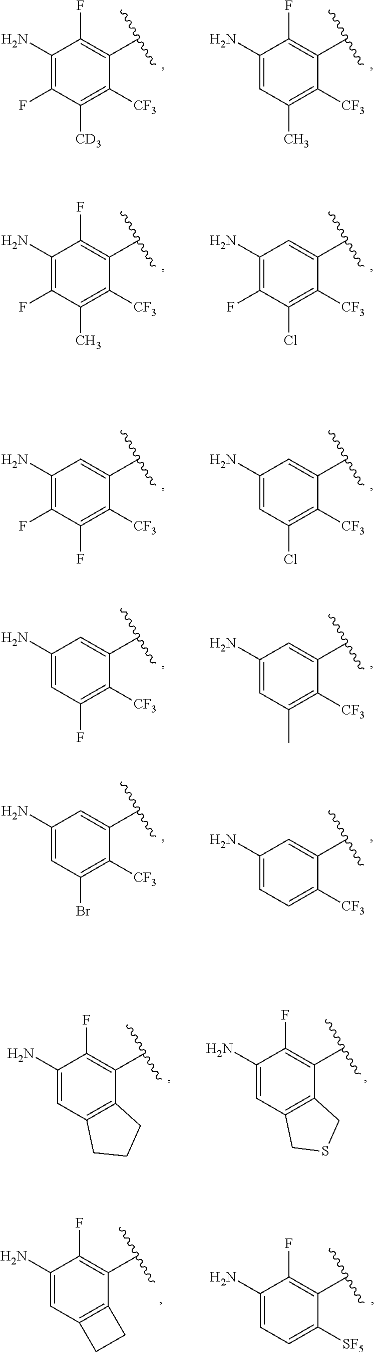

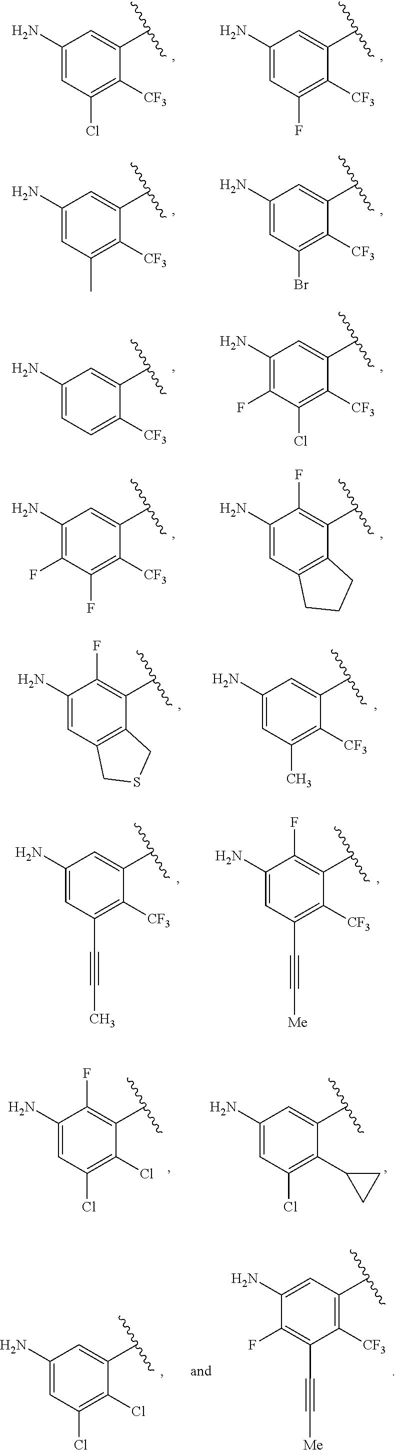

In some embodiments, W-L-R² is selected from:

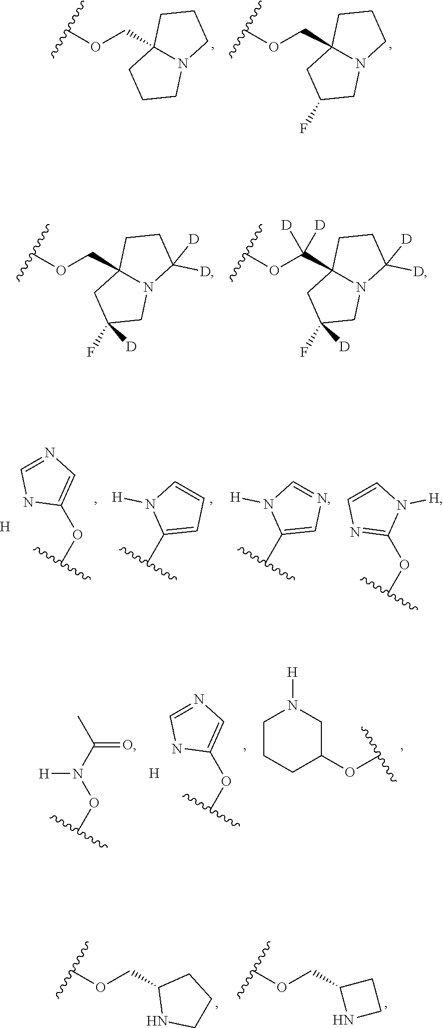

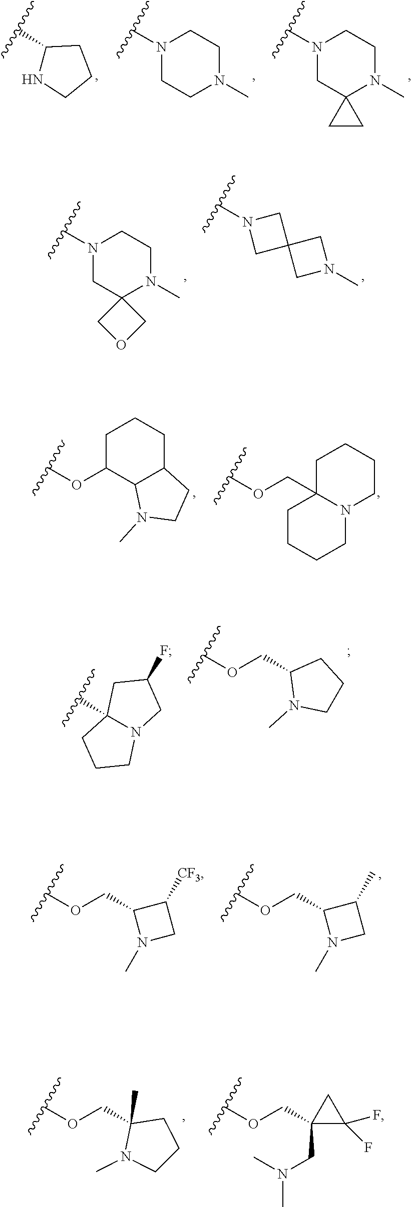

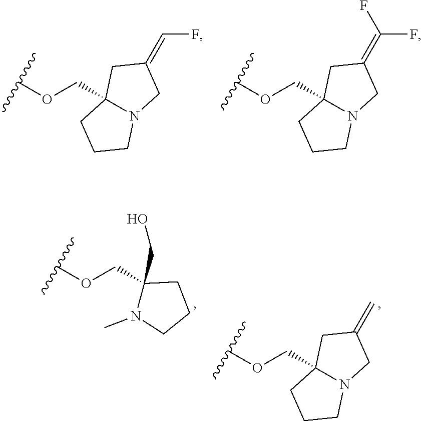 [SMILES: *OC[C@@]12CCCN1C/C(=C\F)C2 ; *OC[C@@]12CCCN1CC(=C(F)F)C2 ; *OC[C@]1(CO)CCCN1C ; *OC[C@@]12CCCN1CC(=C)C2]

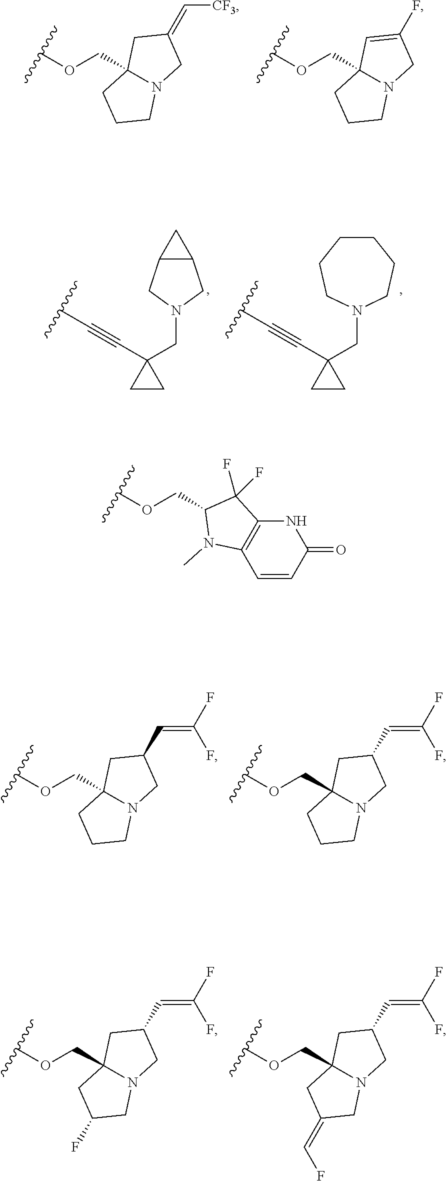

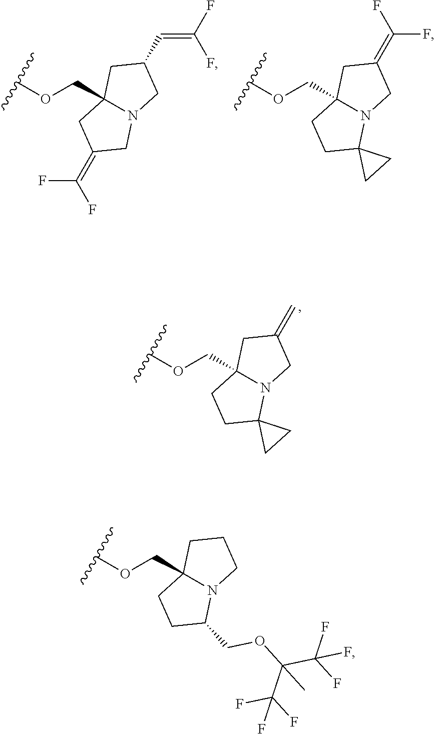

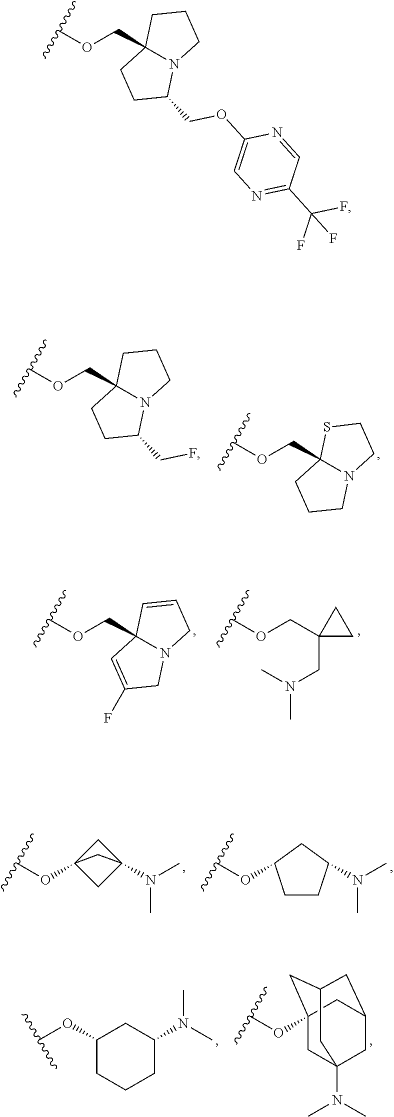

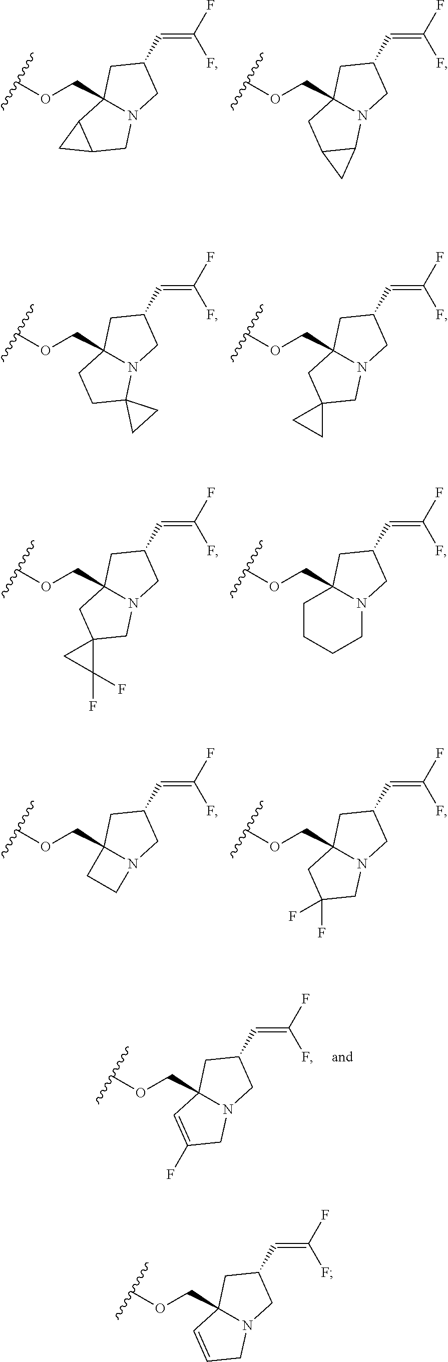

wherein each are optionally substituted with OH, halo, CN, CF₃, C₁₋₄ alkyl, C₁₋₄ alkoxy, and C₁₋₃ hydroxyalkyl.

In some embodiments, G is:

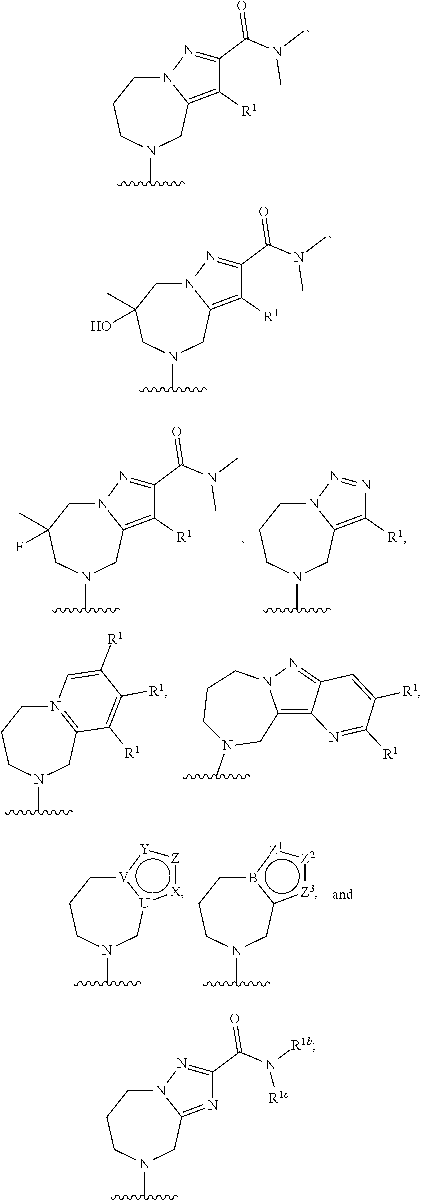

wherein U and V are independently C or N, with the proviso that they are not both N; and X, Y, and Z are independently, CR¹, N, NR¹, O or S; and U, V, X, Y, and Z comprise a heteroaromatic ring; and,
wherein Z¹, Z², Z³ are each independently selected from CR¹, CH, O, N(R¹), NH, N, and S.

In some embodiments, G is selected from:

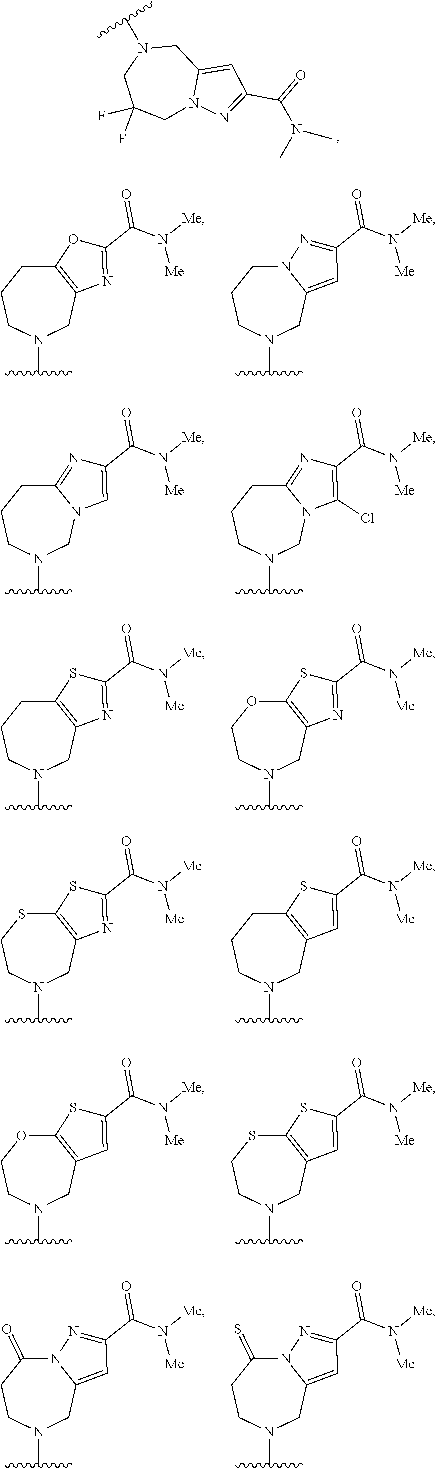

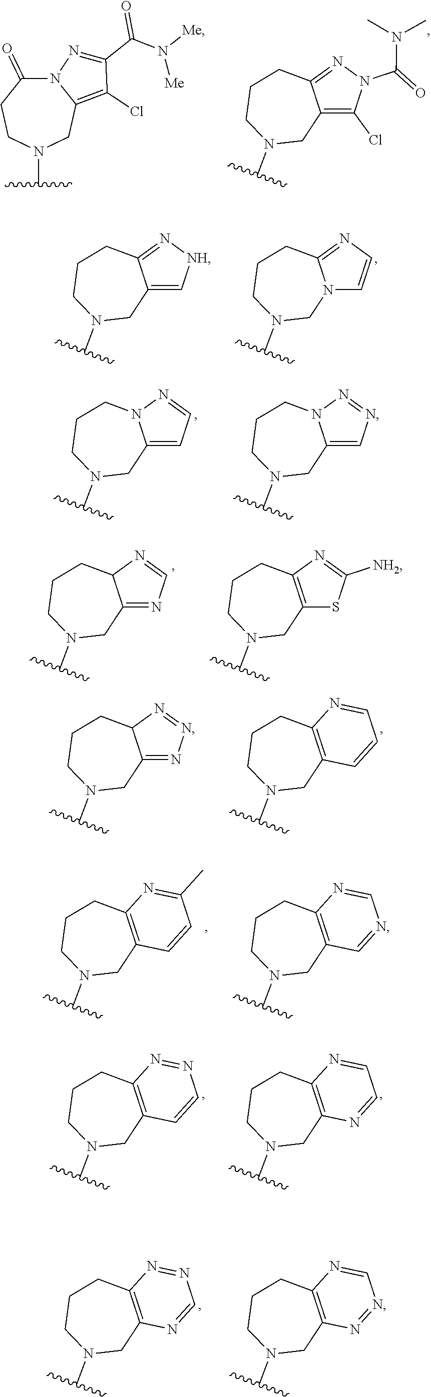

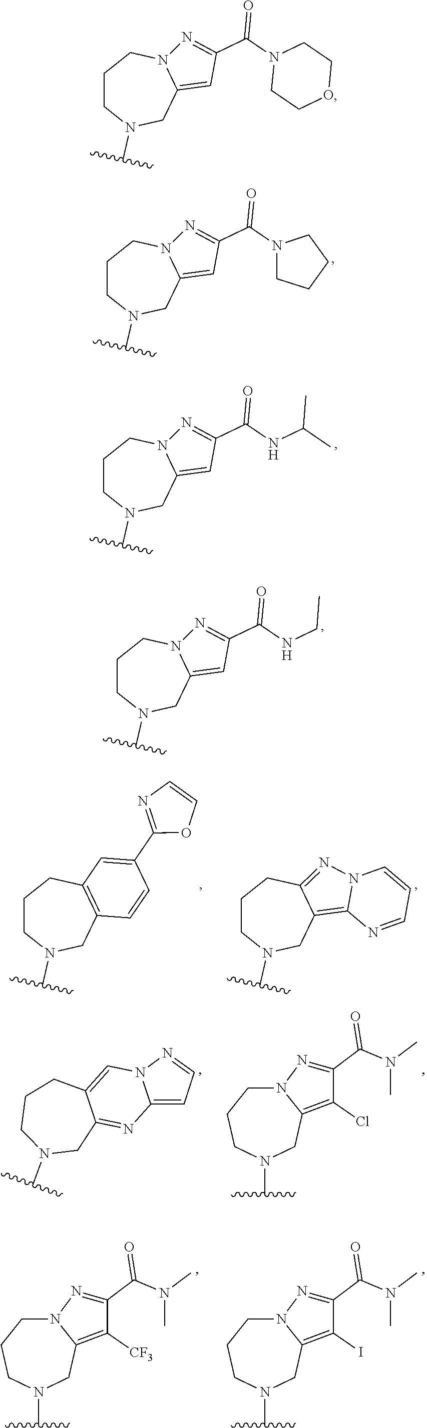

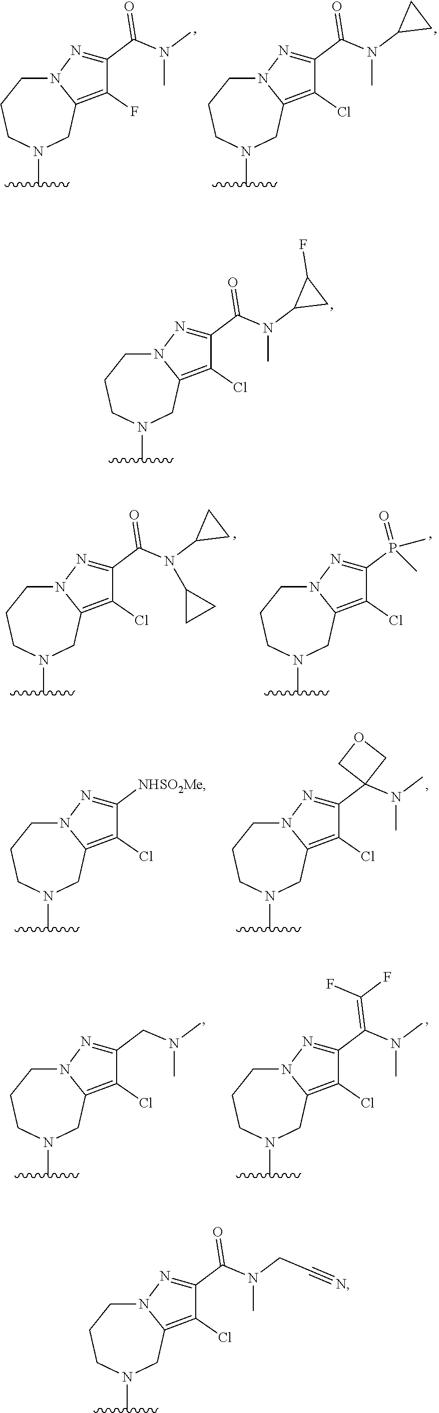

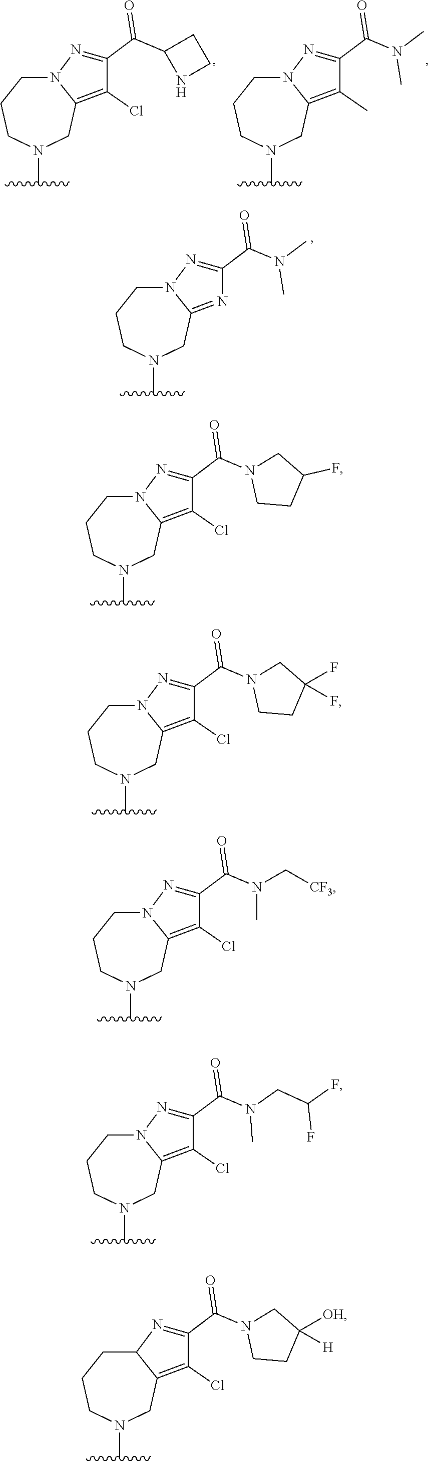

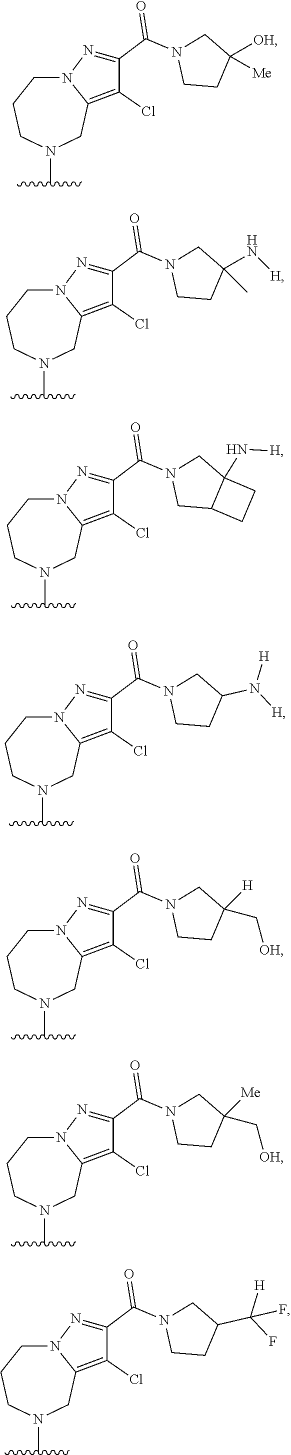

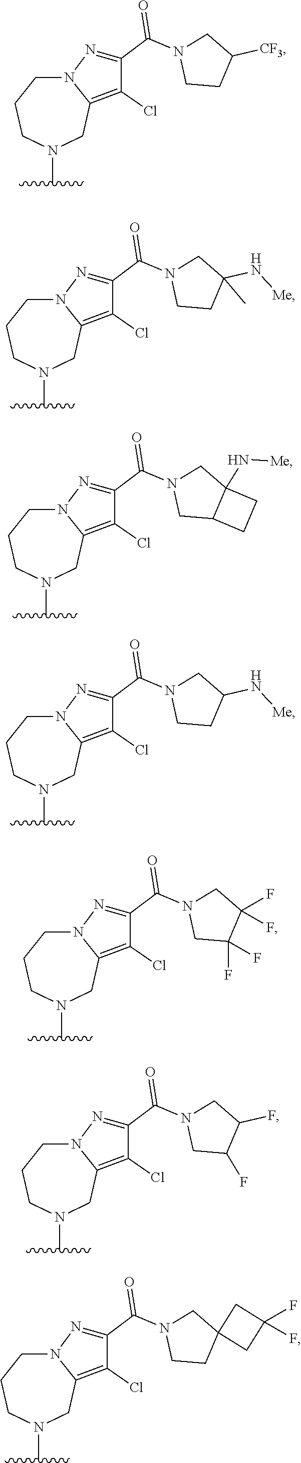

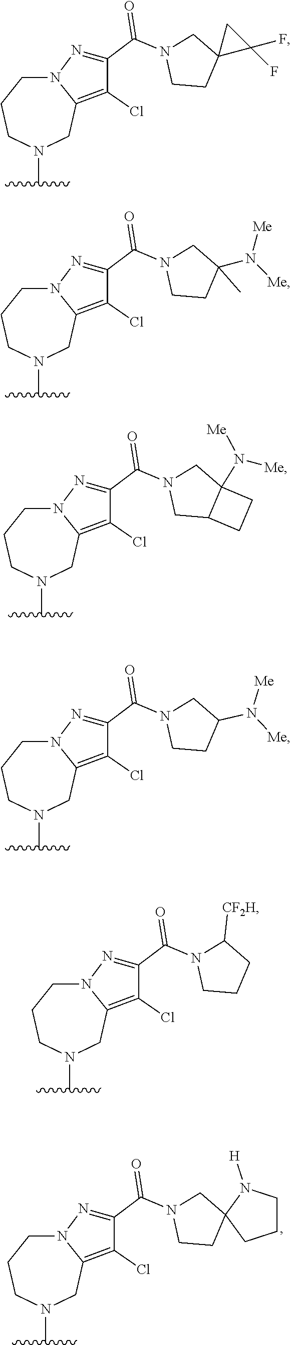 [SMILES: *N1CCCn2nc(C(=O)N3CCC4(C3)CC4(F)F)c(Cl)c2C1 ; *N1CCCn2nc(C(=O)N3CCC(C)(N(C)C)C3)c(Cl)c2C1 ; *N1CCCn2nc(C(=O)N3CC4CCC4(N(C)C)C3)c(Cl)c2C1 ; *N1CCCn2nc(C(=O)N3CCC(N(C)C)C3)c(Cl)c2C1 ; *N1CCCn2nc(C(=O)N3CCCC3C(F)F)c(Cl)c2C1 ; *N1CCCn2nc(C(=O)N3CCC4(CCCN4)C3)c(Cl)c2C1]

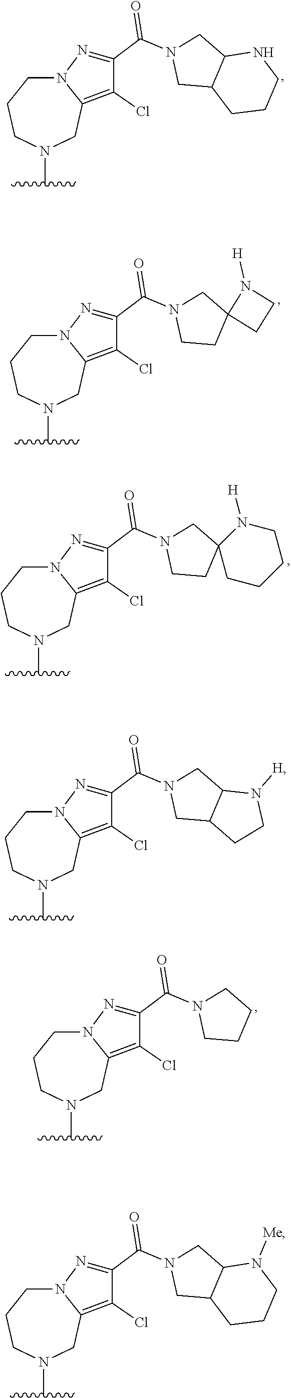

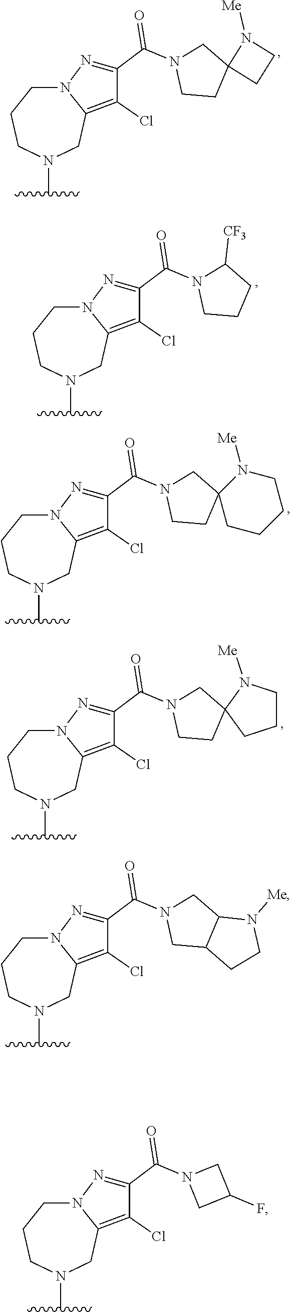 [SMILES: *N1CCCn2nc(C(=O)N3CCC4(CCN4C)C3)c(Cl)c2C1 ; *N1CCCn2nc(C(=O)N3CCCC3C(F)(F)F)c(Cl)c2C1 ; *N1CCCn2nc(C(=O)N3CCC4(CCCCN4C)C3)c(Cl)c2C1 ; *N1CCCn2nc(C(=O)N3CCC4(CCCN4C)C3)c(Cl)c2C1 ; *N1CCCn2nc(C(=O)N3CC4CCN(C)C4C3)c(Cl)c2C1 ; *N1CCCn2nc(C(=O)N3CC(F)C3)c(Cl)c2C1]

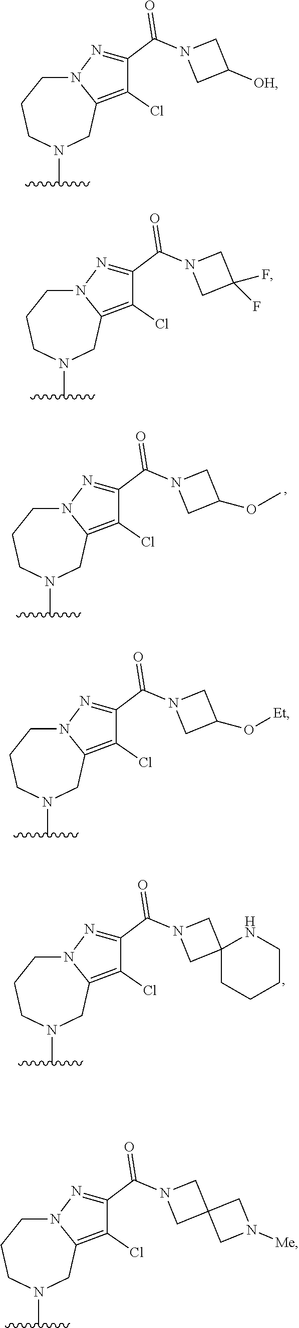 [SMILES: *N1CCCn2nc(C(=O)N3CC(O)C3)c(Cl)c2C1 ; *N1CCCn2nc(C(=O)N3CC(F)(F)C3)c(Cl)c2C1 ; *N1CCCn2nc(C(=O)N3CC(OC)C3)c(Cl)c2C1 ; *N1CCCn2nc(C(=O)N3CC(OCC)C3)c(Cl)c2C1 ; *N1CCCn2nc(C(=O)N3CC4(CCCCN4)C3)c(Cl)c2C1 ; *N1CCCn2nc(C(=O)N3CC4(CN(C)C4)C3)c(Cl)c2C1]

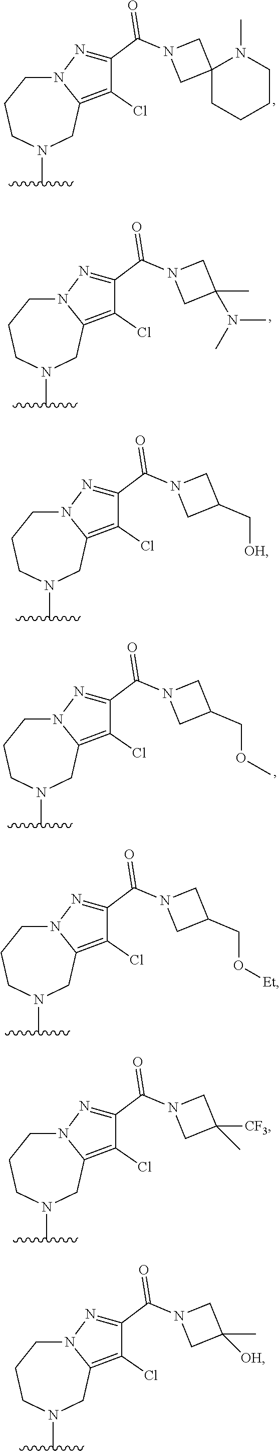

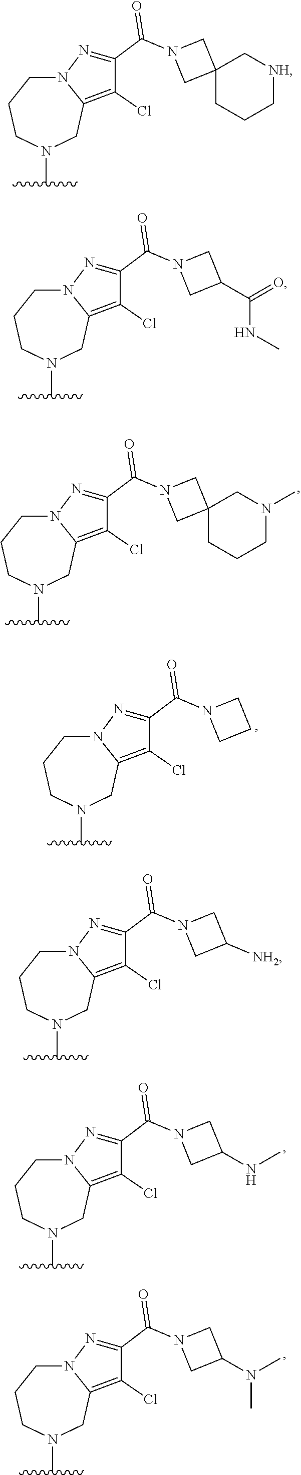

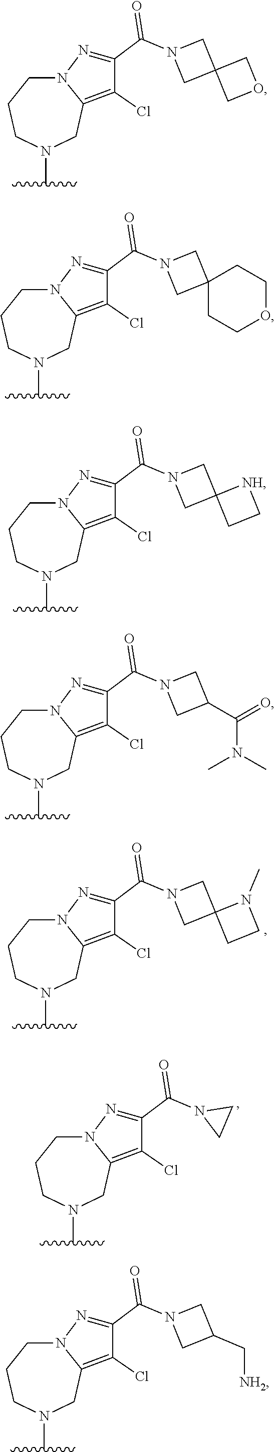

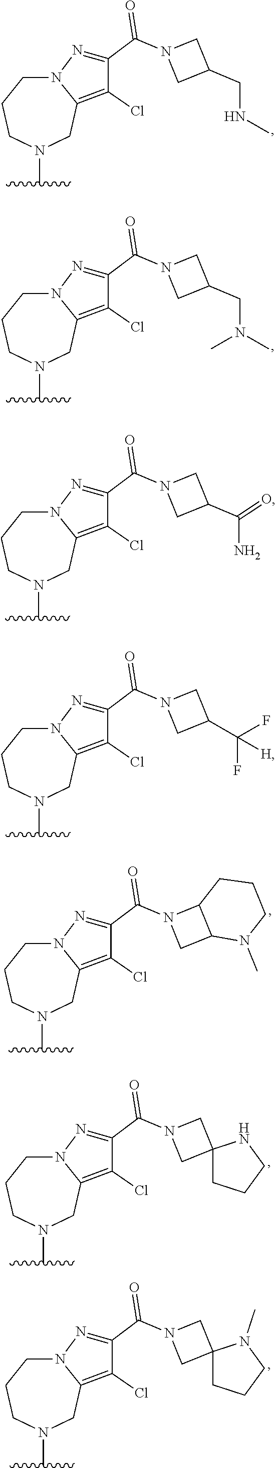

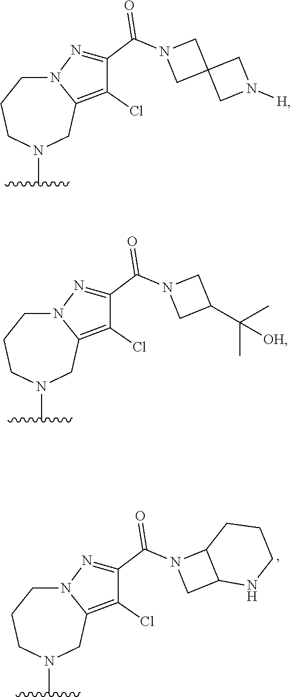

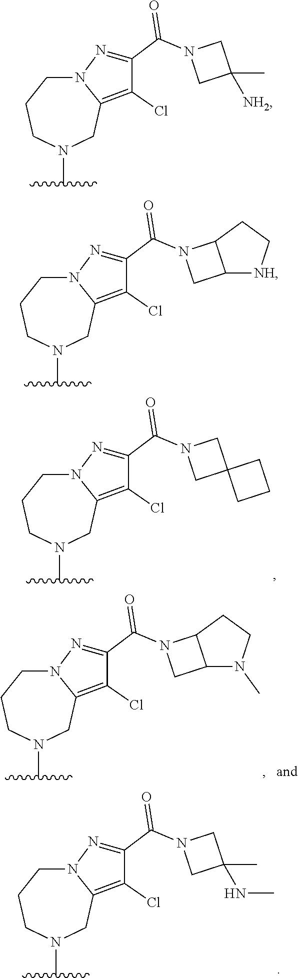 [SMILES: *N1CCCn2nc(C(=O)N3CC(C)(N)C3)c(Cl)c2C1 ; *N1CCCn2nc(C(=O)N3CC4NCCC43)c(Cl)c2C1 ; *N1CCCn2nc(C(=O)N3CC4(CCC4)C3)c(Cl)c2C1 ; *N1CCCn2nc(C(=O)N3CC4C3CCN4C)c(Cl)c2C1 ; *N1CCCn2nc(C(=O)N3CC(C)(NC)C3)c(Cl)c2C1]

In some embodiments, the compound is selected from

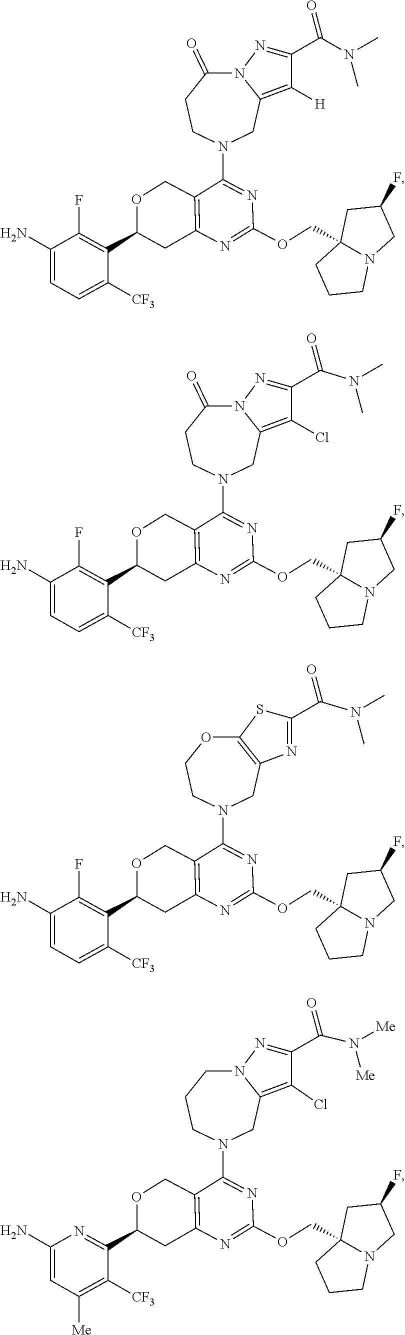

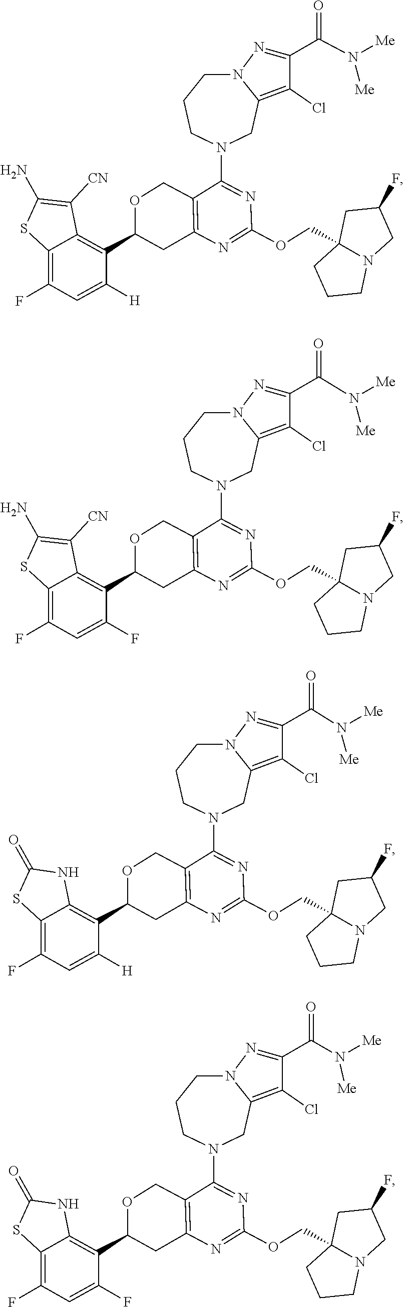

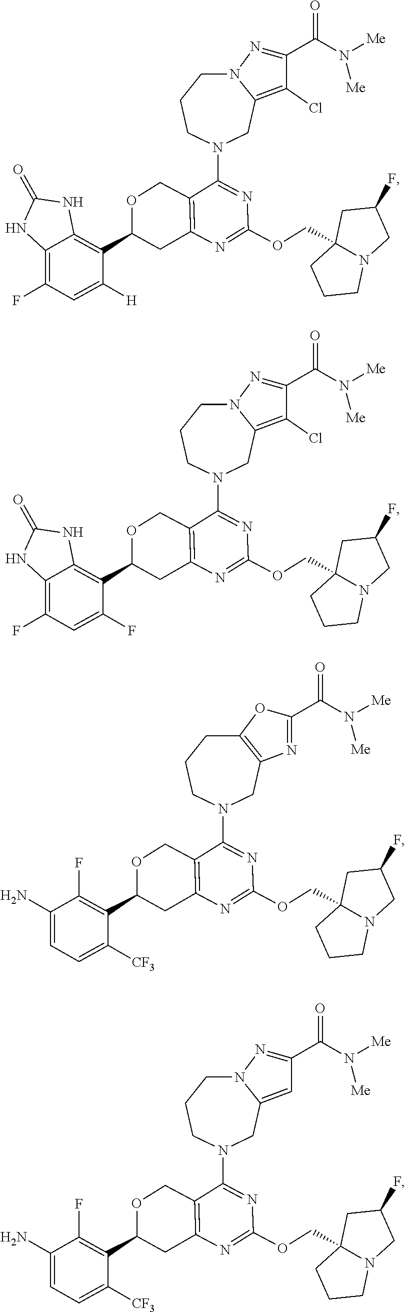

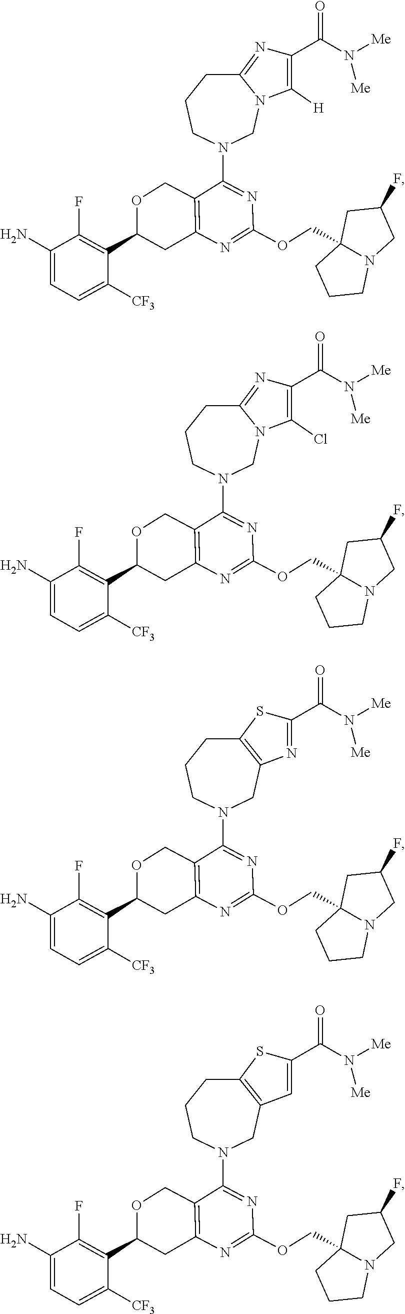

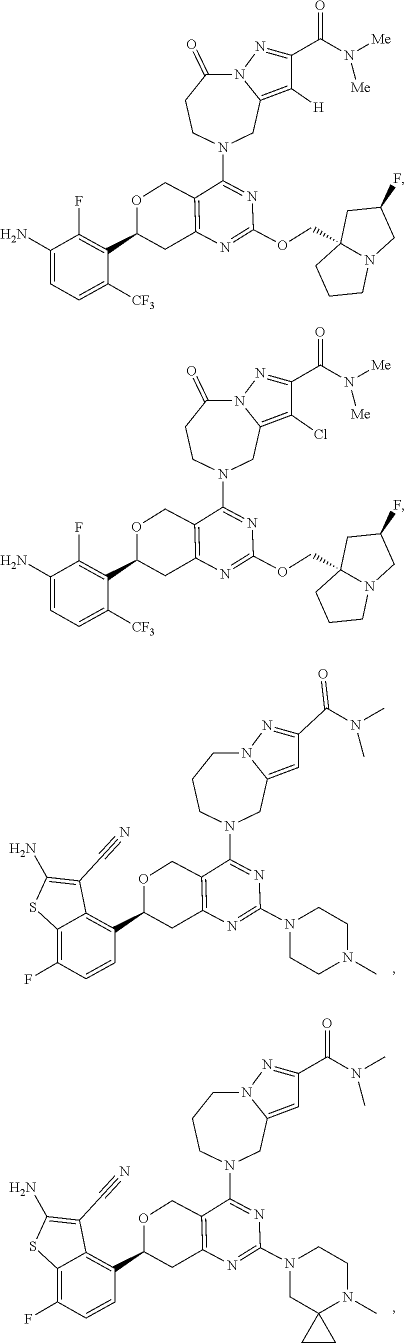

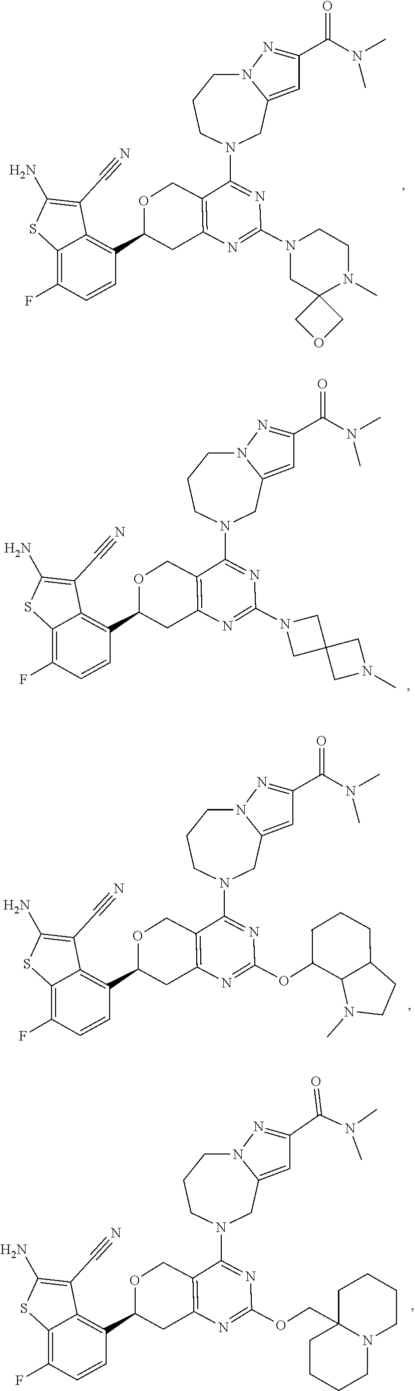

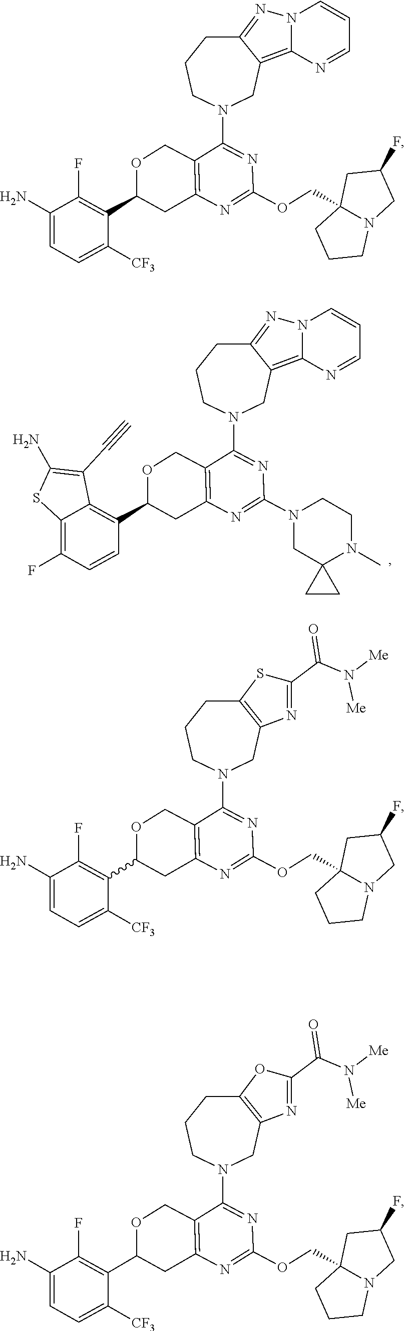

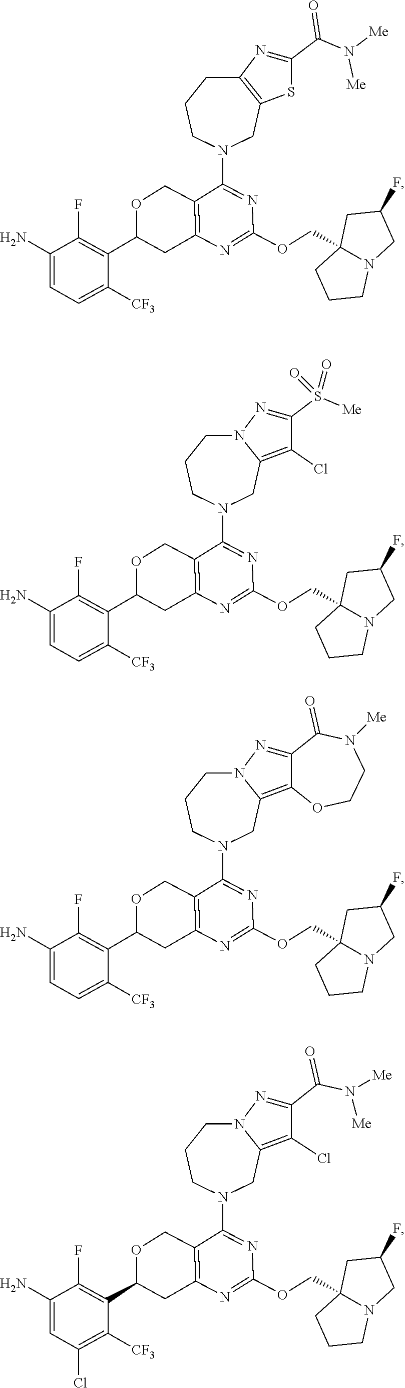 [SMILES: CN(C)C(=O)c1nc2c(s1)CN(c1nc(OC[C@@]34CCCN3C[C@H](F)C4)nc3c1COC(c1c(C(F)(F)F)ccc(N)c1F)C3)CCC2 ; CS(=O)(=O)c1nn2c(c1Cl)CN(c1nc(OC[C@@]34CCCN3C[C@H](F)C4)nc3c1COC(c1c(C(F)(F)F)ccc(N)c1F)C3)CCC2 ; CN1CCOc2c(nn3c2CN(c2nc(OC[C@@]45CCCN4C[C@H](F)C5)nc4c2COC(c2c(C(F)(F)F)ccc(N)c2F)C4)CCC3)C1=O ; CN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2]

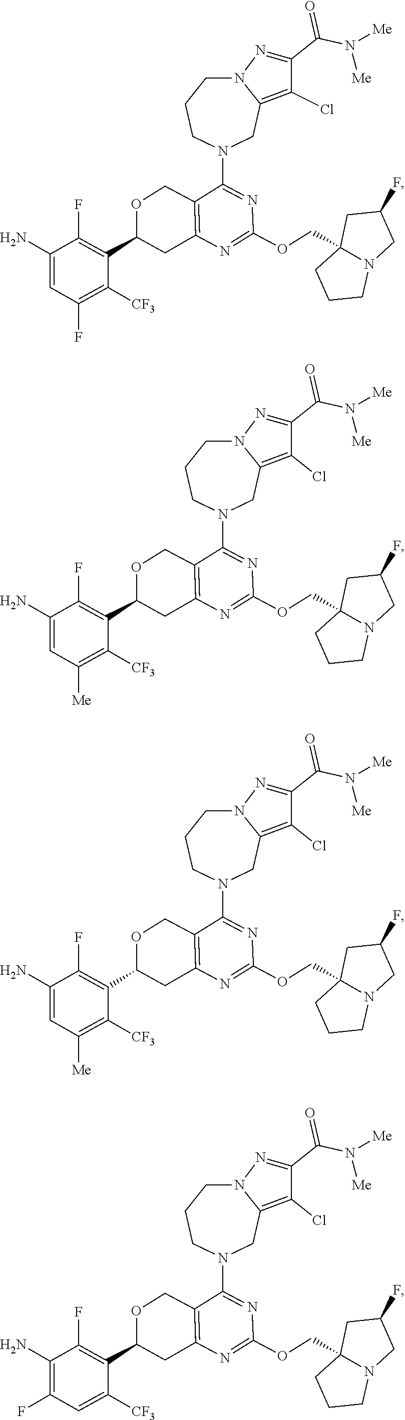 [SMILES: CN(C)C(=O)c1nn2c(c1Cl)CN(c1nc(OC[C@@]34CCCN3C[C@H](F)C4)nc3c1CO[C@H](c1c(F)c(N)cc(F)c1C(F)(F)F)C3)CCC2 ; Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F ; Cc1cc(N)c(F)c([C@H]2Cc3nc(OC[C@@]45CCCN4C[C@H](F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C[C@H](F)C4)nc3c1CO[C@H](c1c(C(F)(F)F)cc(F)c(N)c1F)C3)CCC2]

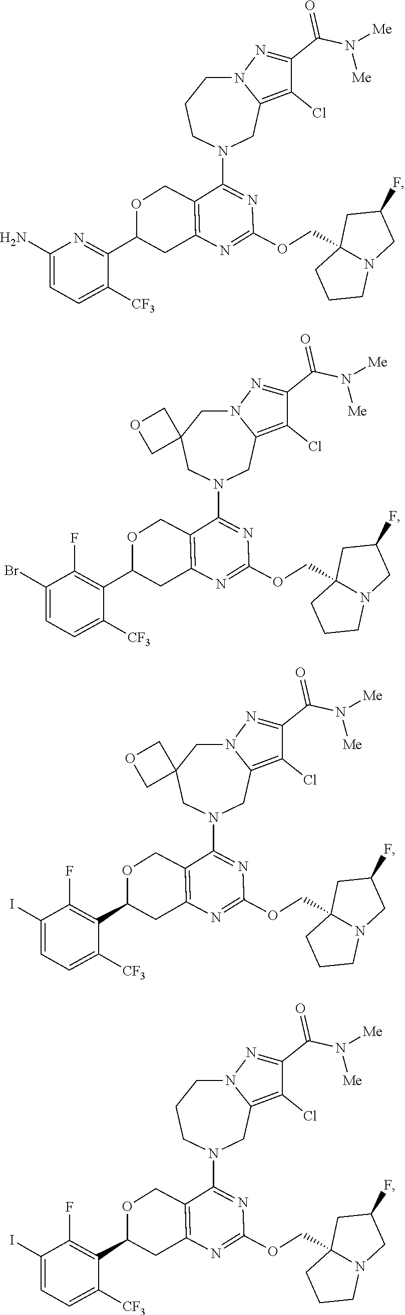

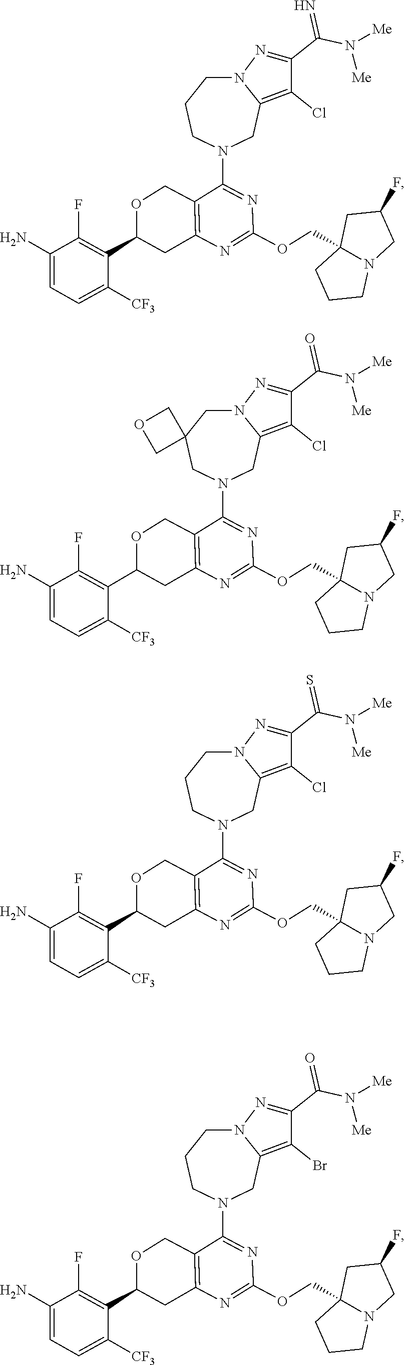

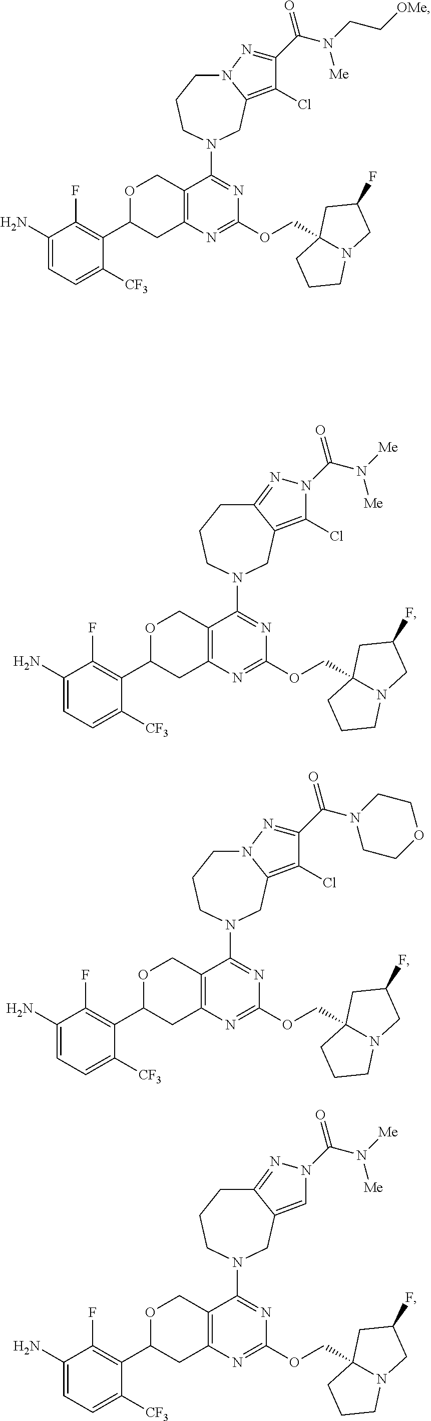

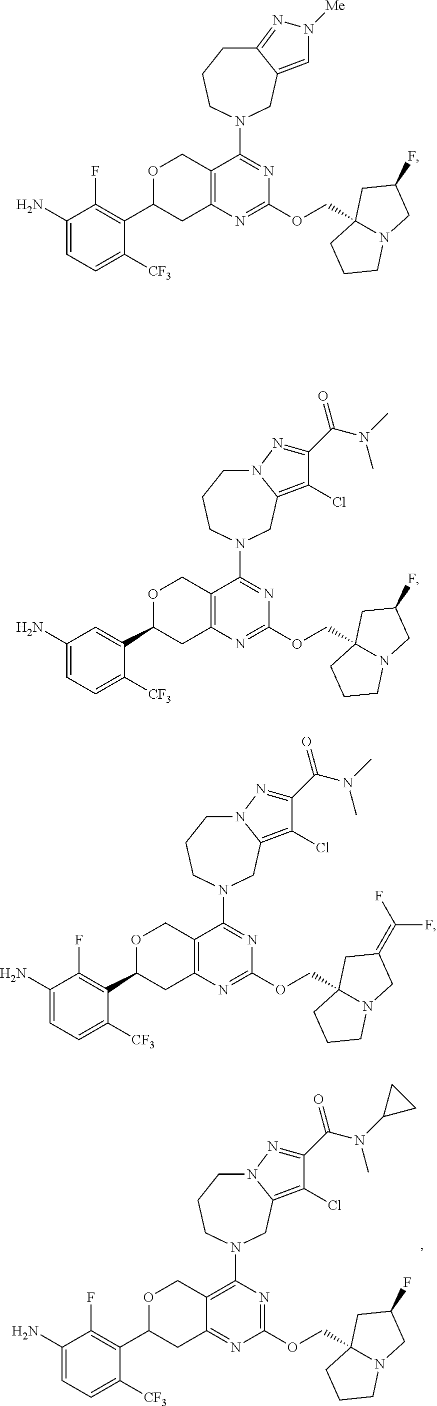 [SMILES: Cn1cc2c(n1)CCCN(c1nc(OC[C@@]34CCCN3C[C@H](F)C4)nc3c1COC(c1c(C(F)(F)F)ccc(N)c1F)C3)C2 ; CN(C)C(=O)c1nn2c(c1Cl)CN(c1nc(OC[C@@]34CCCN3C[C@H](F)C4)nc3c1CO[C@H](c1cc(N)ccc1C(F)(F)F)C3)CCC2 ; CN(C)C(=O)c1nn2c(c1Cl)CN(c1nc(OC[C@@]34CCCN3CC(=C(F)F)C4)nc3c1CO[C@H](c1c(C(F)(F)F)ccc(N)c1F)C3)CCC2 ; CN(C(=O)c1nn2c(c1Cl)CN(c1nc(OC[C@@]34CCCN3C[C@H](F)C4)nc3c1COC(c1c(C(F)(F)F)ccc(N)c1F)C3)CCC2)C1CC1]

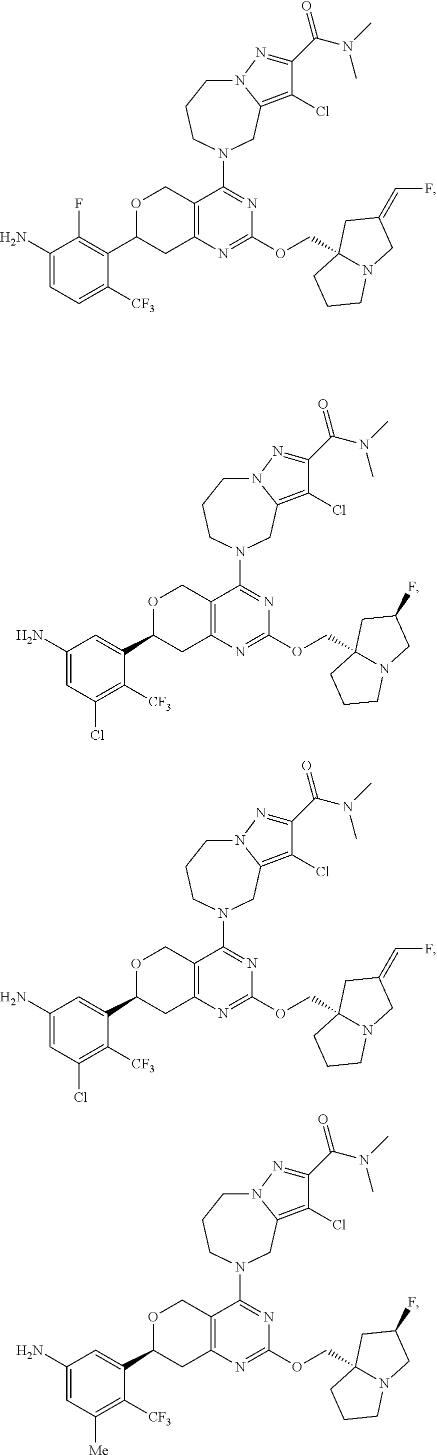 [SMILES: CN(C)C(=O)c1nn2c(c1Cl)CN(c1nc(OC[C@@]34CCCN3C/C(=C\F)C4)nc3c1COC(c1c(C(F)(F)F)ccc(N)c1F)C3)CCC2 ; CN(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1CO[C@H](c1cc(N)cc(Cl)c1C(F)(F)F)C3)CCC2 ; Cc1cc(N)cc([C@@H]2Cc3nc(OC[C@@]45CCCN4C[C@H](F)C5)nc(N4CCCn5nc(C(=O)N(C)C)c(Cl)c5C4)c3CO2)c1C(F)(F)F]

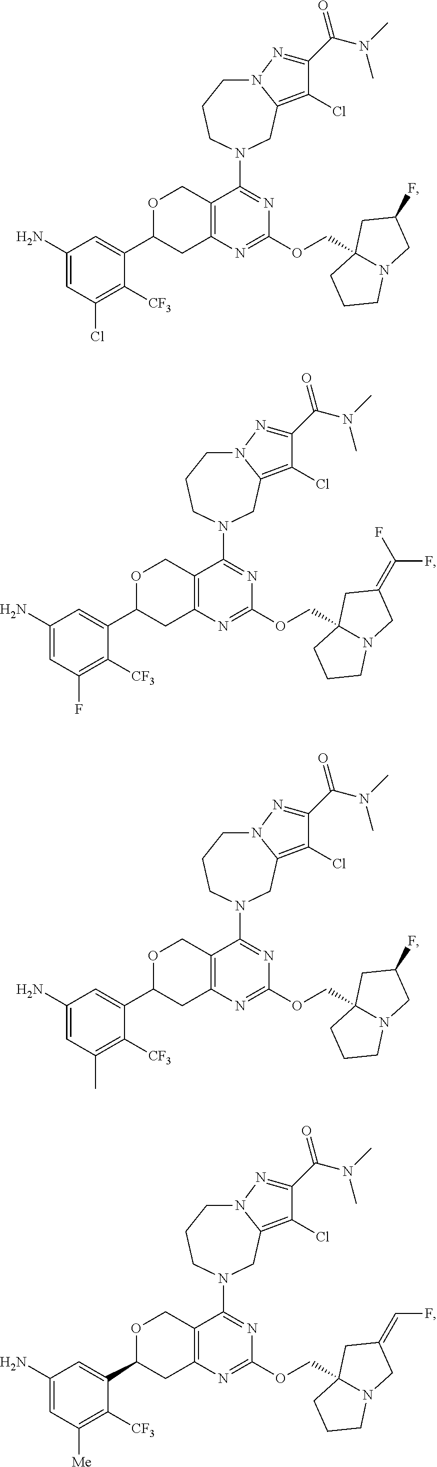 [SMILES: CN(C)C(=O)c1nn2c(c1Cl)CN(c1nc(OC[C@@]34CCCN3C[C@H](F)C4)nc3c1COC(c1cc(N)cc(Cl)c1C(F)(F)F)C3)CCC2 ; CN(C)C(=O)c1nn2c(c1Cl)CN(c1nc(OC[C@@]34CCCN3CC(=C(F)F)C4)nc3c1COC(c1cc(N)cc(F)c1C(F)(F)F)C3)CCC2 ; Cc1cc(N)cc(C2Cc3nc(OC[C@@]45CCCN4C[C@H](F)C5)nc(N4CCCn5nc(C(=O)N(C)C)c(Cl)c5C4)c3CO2)c1C(F)(F)F ; Cc1cc(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F]

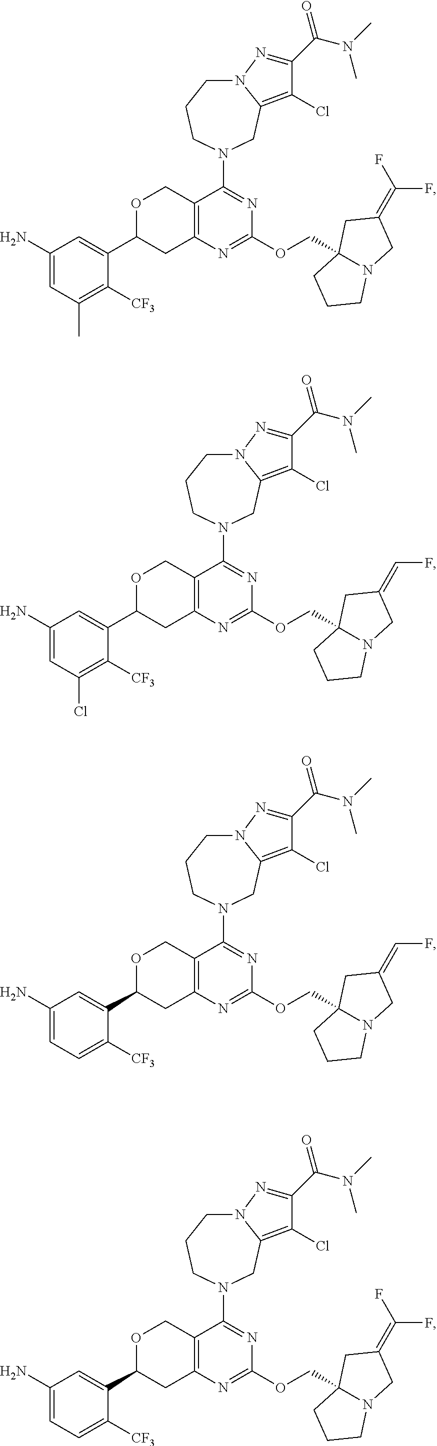 [SMILES: Cc1cc(N)cc(C2Cc3nc(OC[C@@]45CCCN4CC(=C(F)F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1CO[C@H](c1cc(N)ccc1C(F)(F)F)C3)CCC2 ; CN(C)C(=O)c1nn2c(c1Cl)CN(c1nc(OC[C@@]34CCCN3CC(=C(F)F)C4)nc3c1CO[C@H](c1cc(N)ccc1C(F)(F)F)C3)CCC2]

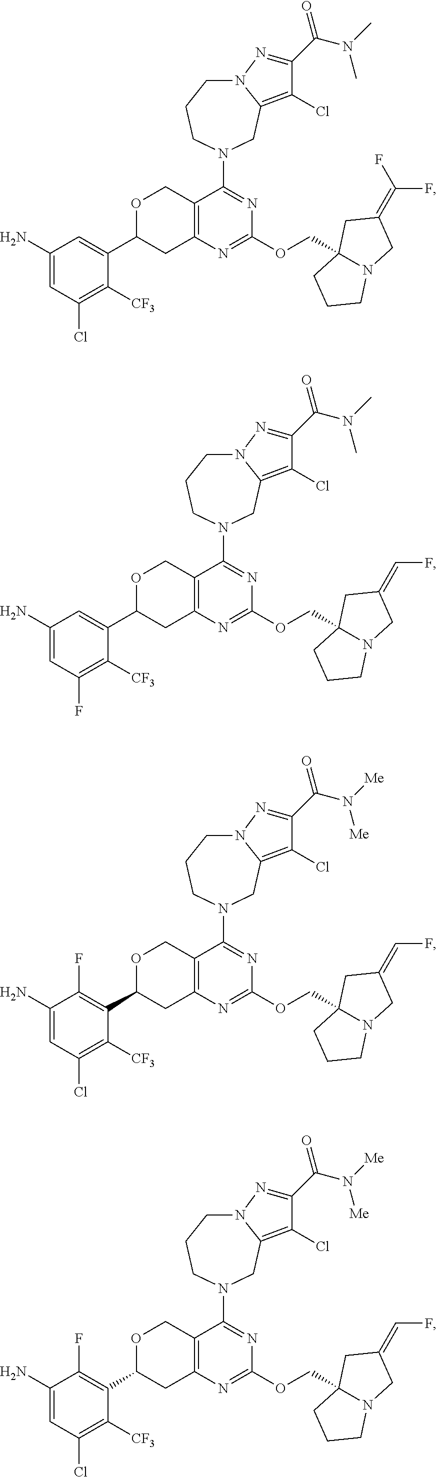 [SMILES: CN(C)C(=O)c1nn2c(c1Cl)CN(c1nc(OC[C@@]34CCCN3CC(=C(F)F)C4)nc3c1COC(c1cc(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1COC(c1cc(N)cc(F)c1C(F)(F)F)C3)CCC2 ; CN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1CO[C@@H](c1c(F)c(N)cc(Cl)c1C(F)(F)F)C3)CCC2]

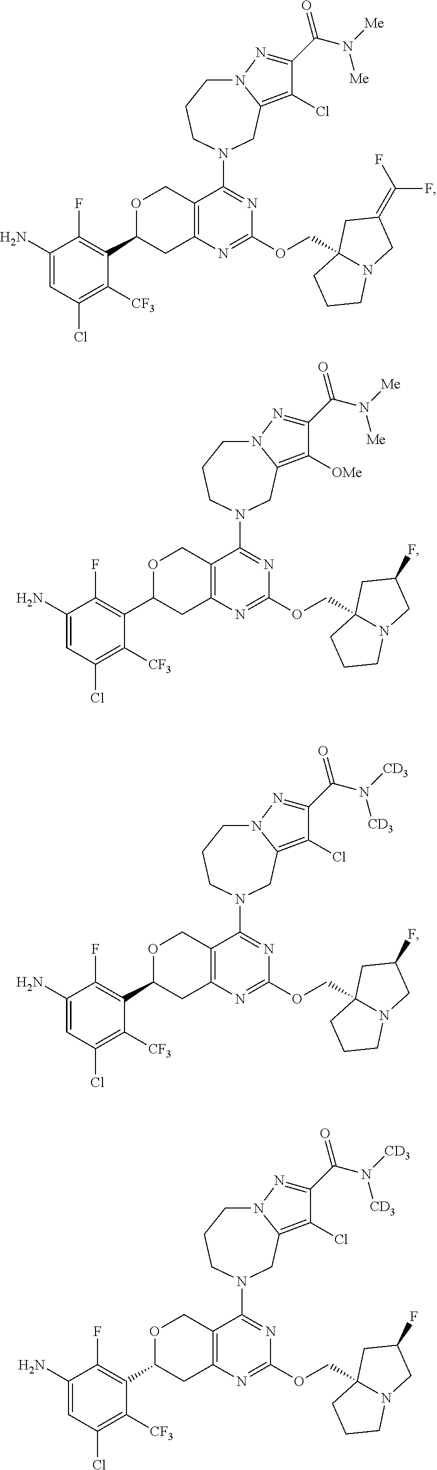 [SMILES: CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2 ; COc1c(C(=O)N(C)C)nn2c1CN(c1nc(OC[C@@]34CCCN3C[C@H](F)C4)nc3c1COC(c1c(F)c(N)cc(Cl)c1C(F)(F)F)C3)CCC2 ; [2H]C([2H])([2H])N(C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2)C([2H])([2H])[2H] ; [2H]C([2H])([2H])N(C(=O)c1nn2c(c1Cl)CN(c1nc(OC[C@@]34CCCN3C[C@H](F)C4)nc3c1CO[C@@H](c1c(F)c(N)cc(Cl)c1C(F)(F)F)C3)CCC2)C([2H])([2H])[2H]]

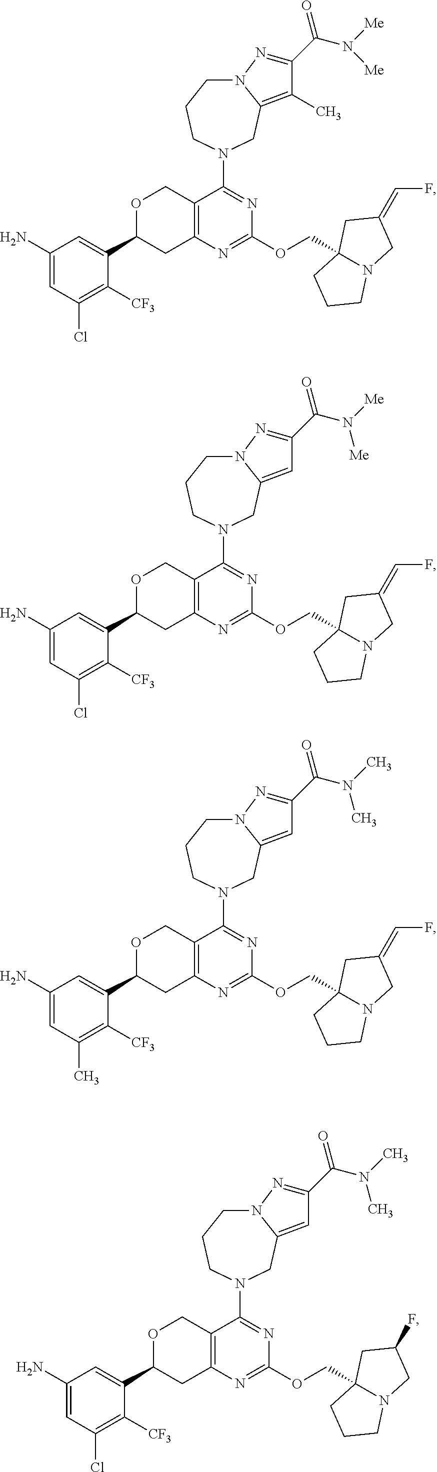 [SMILES: Cc1c(C(=O)N(C)C)nn2c1CN(c1nc(OC[C@@]34CCCN3C/C(=C\F)C4)nc3c1CO[C@H](c1cc(N)cc(Cl)c1C(F)(F)F)C3)CCC2 ; CN(C)C(=O)c1cc2n(n1)CCCN(c1nc(OC[C@@]34CCCN3C/C(=C\F)C4)nc3c1CO[C@H](c1cc(N)cc(Cl)c1C(F)(F)F)C3)C2 ; Cc1cc(N)cc([C@@H]2Cc3nc(OC[C@@]45CCCN4C/C(=C\F)C5)nc(N4CCCn5nc(C(=O)N(C)C)cc5C4)c3CO2)c1C(F)(F)F ; CN(C)C(=O)c1cc2n(n1)CCCN(c1nc(OC[C@@]34CCCN3C[C@H](F)C4)nc3c1CO[C@H](c1cc(N)cc(Cl)c1C(F)(F)F)C3)C2]

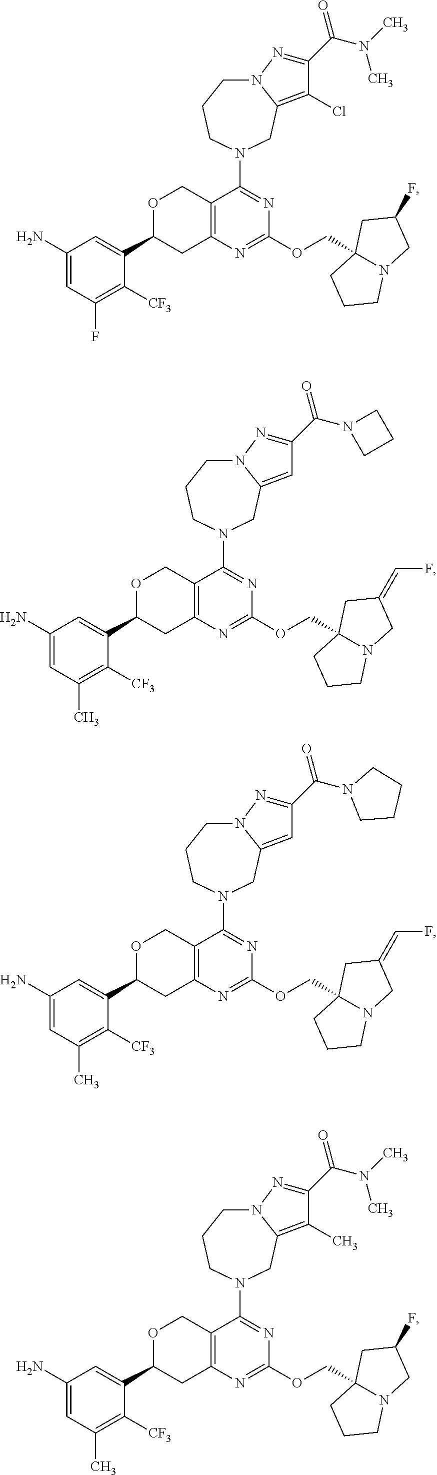 [SMILES: CN(C)C(=O)c1nn2c(c1Cl)CN(c1nc(OC[C@@]34CCCN3C[C@H](F)C4)nc3c1CO[C@H](c1cc(N)cc(F)c1C(F)(F)F)C3)CCC2 ; Cc1cc(N)cc([C@@H]2Cc3nc(OC[C@@]45CCCN4C/C(=C\F)C5)nc(N4CCCn5nc(C(=O)N6CCC6)cc5C4)c3CO2)c1C(F)(F)F ; Cc1cc(N)cc([C@@H]2Cc3nc(OC[C@@]45CCCN4C/C(=C\F)C5)nc(N4CCCn5nc(C(=O)N6CCCC6)cc5C4)c3CO2)c1C(F)(F)F ; Cc1cc(N)cc([C@@H]2Cc3nc(OC[C@@]45CCCN4C[C@H](F)C5)nc(N4CCCn5nc(C(=O)N(C)C)c(C)c5C4)c3CO2)c1C(F)(F)F]

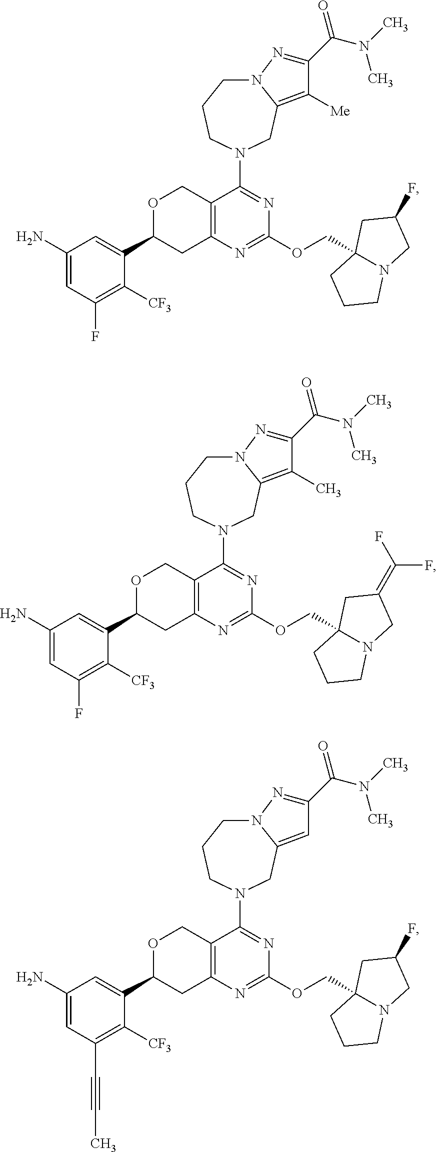 [SMILES: Cc1c(C(=O)N(C)C)nn2c1CN(c1nc(OC[C@@]34CCCN3C[C@H](F)C4)nc3c1CO[C@H](c1cc(N)cc(F)c1C(F)(F)F)C3)CCC2 ; Cc1c(C(=O)N(C)C)nn2c1CN(c1nc(OC[C@@]34CCCN3CC(=C(F)F)C4)nc3c1CO[C@H](c1cc(N)cc(F)c1C(F)(F)F)C3)CCC2 ; CC#Cc1cc(N)cc([C@@H]2Cc3nc(OC[C@@]45CCCN4C[C@H](F)C5)nc(N4CCCn5nc(C(=O)N(C)C)cc5C4)c3CO2)c1C(F)(F)F]

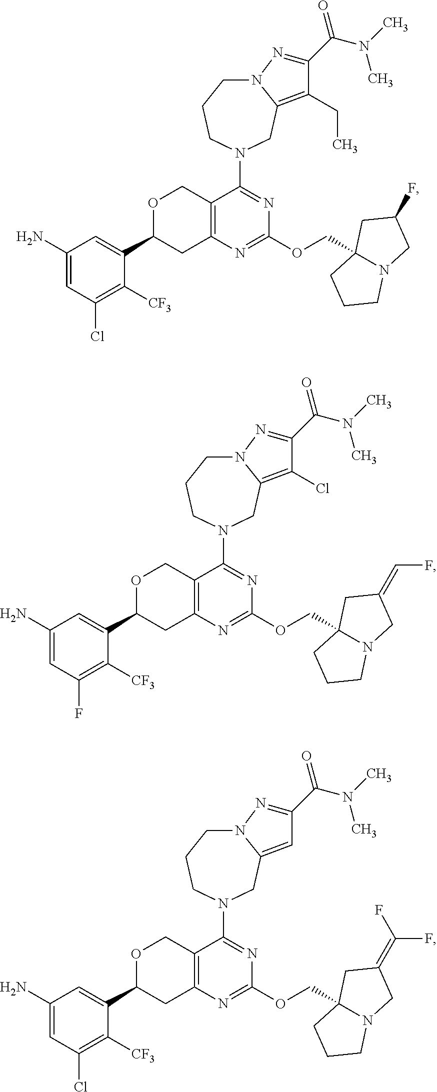 [SMILES: CCc1c(C(=O)N(C)C)nn2c1CN(c1nc(OC[C@@]34CCCN3C[C@H](F)C4)nc3c1CO[C@H](c1cc(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1CO[C@H](c1cc(N)cc(F)c1C(F)(F)F)C3)CCC2 ; CN(C)C(=O)c1cc2n(n1)CCCN(c1nc(OC[C@@]34CCCN3CC(=C(F)F)C4)nc3c1CO[C@H](c1cc(N)cc(Cl)c1C(F)(F)F)C3)C2]

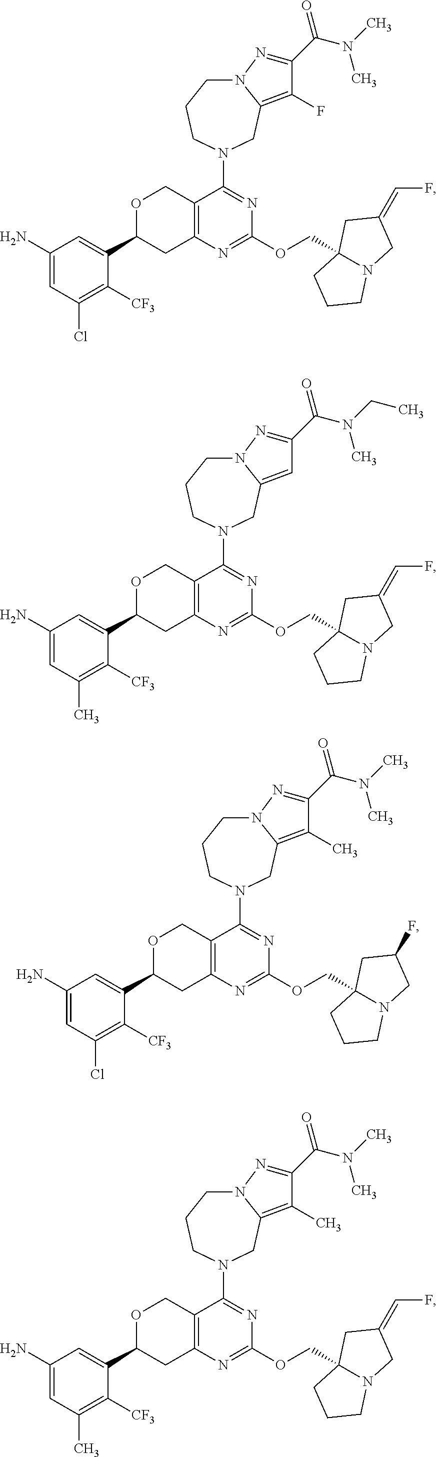 [SMILES: CN(C)C(=O)c1nn2c(c1F)CN(c1nc(OC[C@@]34CCCN3C/C(=C\F)C4)nc3c1CO[C@H](c1cc(N)cc(Cl)c1C(F)(F)F)C3)CCC2 ; CCN(C)C(=O)c1cc2n(n1)CCCN(c1nc(OC[C@@]34CCCN3C/C(=C\F)C4)nc3c1CO[C@H](c1cc(N)cc(C)c1C(F)(F)F)C3)C2 ; Cc1c(C(=O)N(C)C)nn2c1CN(c1nc(OC[C@@]34CCCN3C[C@H](F)C4)nc3c1CO[C@H](c1cc(N)cc(Cl)c1C(F)(F)F)C3)CCC2 ; Cc1cc(N)cc([C@@H]2Cc3nc(OC[C@@]45CCCN4C/C(=C\F)C5)nc(N4CCCn5nc(C(=O)N(C)C)c(C)c5C4)c3CO2)c1C(F)(F)F]

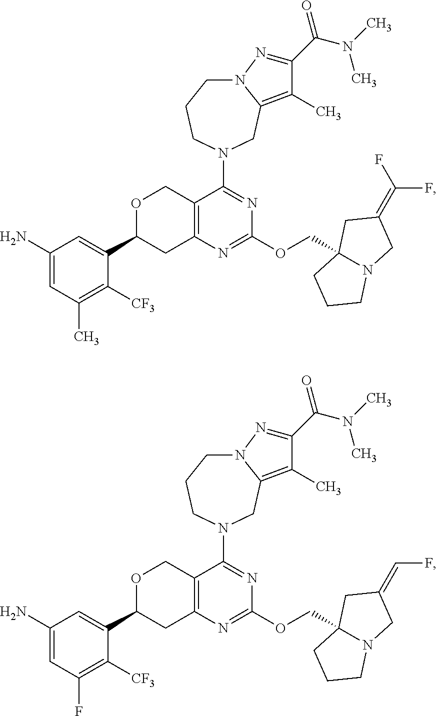 [SMILES: Cc1cc(N)cc([C@@H]2Cc3nc(OC[C@@]45CCCN4CC(=C(F)F)C5)nc(N4CCCn5nc(C(=O)N(C)C)c(C)c5C4)c3CO2)c1C(F)(F)F ; Cc1c(C(=O)N(C)C)nn2c1CN(c1nc(OC[C@@]34CCCN3C/C(=C\F)C4)nc3c1CO[C@H](c1cc(N)cc(F)c1C(F)(F)F)C3)CCC2]

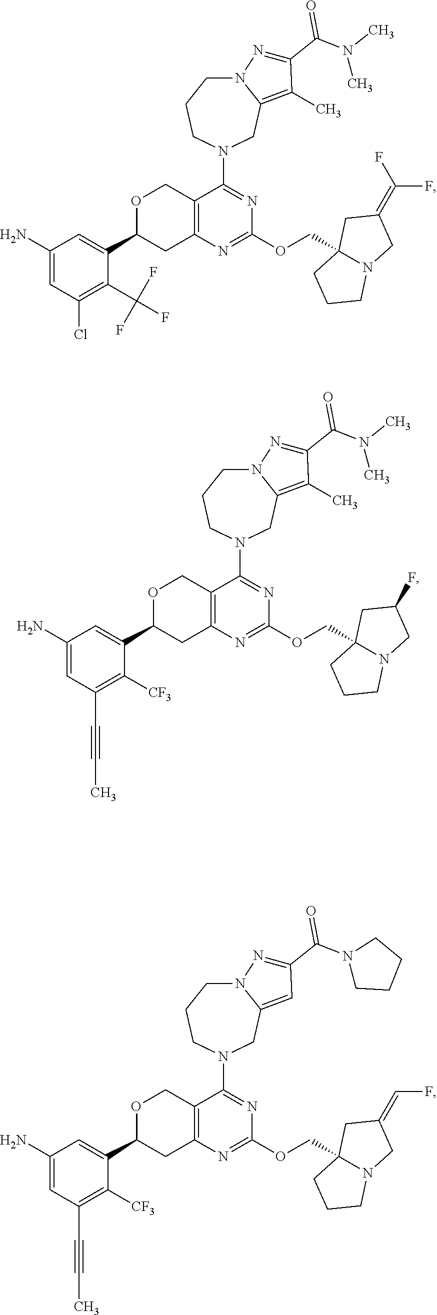 [SMILES: Cc1c(C(=O)N(C)C)nn2c1CN(c1nc(OC[C@@]34CCCN3CC(=C(F)F)C4)nc3c1CO[C@H](c1cc(N)cc(Cl)c1C(F)(F)F)C3)CCC2 ; CC#Cc1cc(N)cc([C@@H]2Cc3nc(OC[C@@]45CCCN4C[C@H](F)C5)nc(N4CCCn5nc(C(=O)N(C)C)c(C)c5C4)c3CO2)c1C(F)(F)F ; CC#Cc1cc(N)cc([C@@H]2Cc3nc(OC[C@@]45CCCN4C/C(=C\F)C5)nc(N4CCCn5nc(C(=O)N6CCCC6)cc5C4)c3CO2)c1C(F)(F)F]

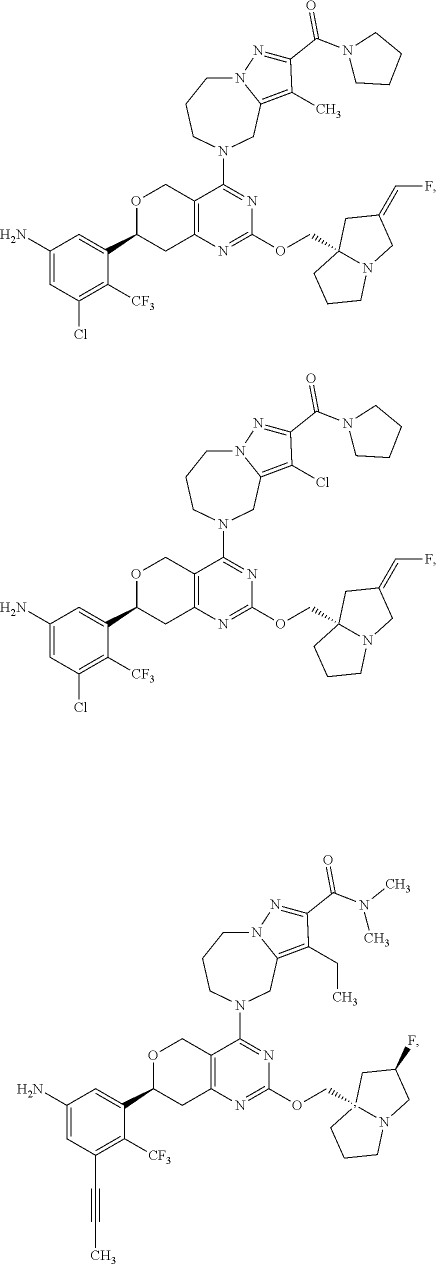

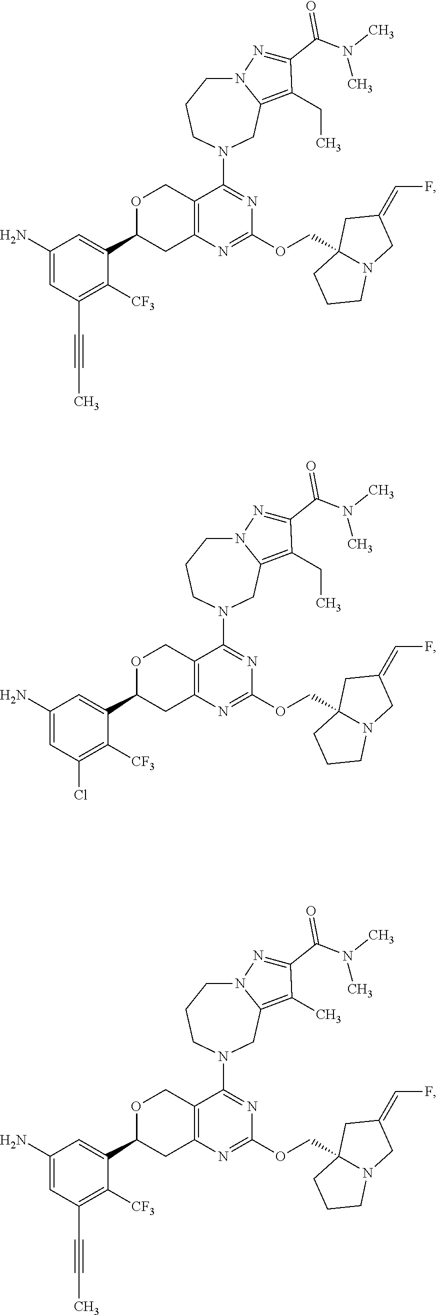 [SMILES: CC#Cc1cc(N)cc([C@@H]2Cc3nc(OC[C@@]45CCCN4C/C(=C\F)C5)nc(N4CCCn5nc(C(=O)N(C)C)c(CC)c5C4)c3CO2)c1C(F)(F)F ; CCc1c(C(=O)N(C)C)nn2c1CN(c1nc(OC[C@@]34CCCN3C/C(=C\F)C4)nc3c1CO[C@H](c1cc(N)cc(Cl)c1C(F)(F)F)C3)CCC2 ; CC#Cc1cc(N)cc([C@@H]2Cc3nc(OC[C@@]45CCCN4C/C(=C\F)C5)nc(N4CCCn5nc(C(=O)N(C)C)c(C)c5C4)c3CO2)c1C(F)(F)F]

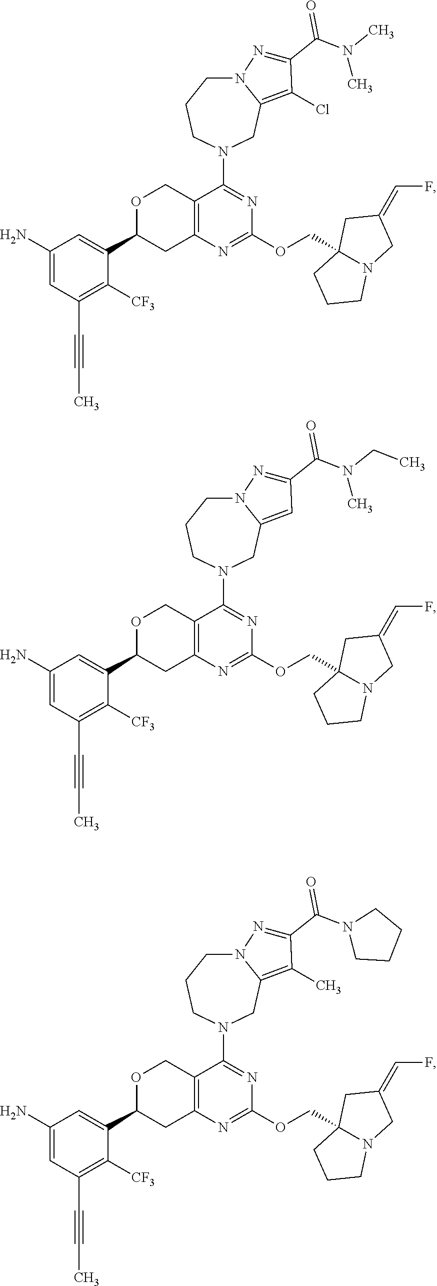 [SMILES: CC#Cc1cc(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F ; CC#Cc1cc(N)cc([C@@H]2Cc3nc(OC[C@@]45CCCN4C/C(=C\F)C5)nc(N4CCCn5nc(C(=O)N(C)CC)cc5C4)c3CO2)c1C(F)(F)F ; CC#Cc1cc(N)cc([C@@H]2Cc3nc(OC[C@@]45CCCN4C/C(=C\F)C5)nc(N4CCCn5nc(C(=O)N6CCCC6)c(C)c5C4)c3CO2)c1C(F)(F)F]

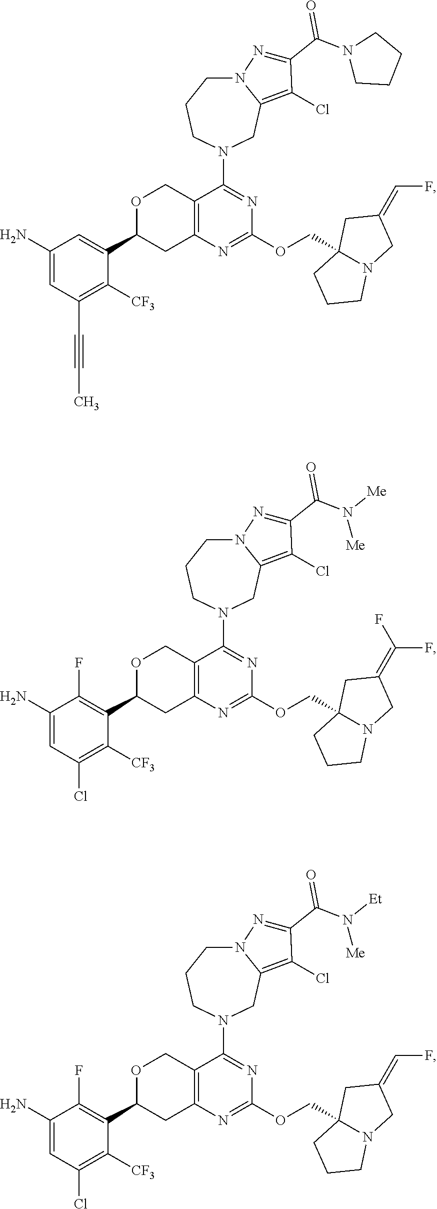 [SMILES: CC#Cc1cc(N)cc([C@@H]2Cc3nc(OC[C@@]45CCCN4C/C(=C\F)C5)nc(N4CCCn5nc(C(=O)N6CCCC6)c(Cl)c5C4)c3CO2)c1C(F)(F)F ; CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2 ; CCN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2]

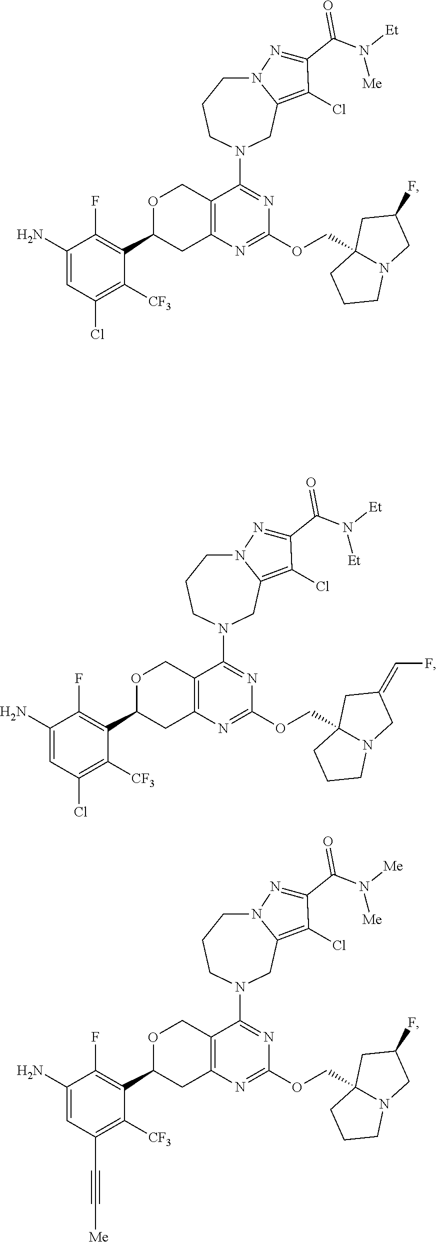 [SMILES: CCN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2 ; CCN(CC)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2 ; CC#Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F]

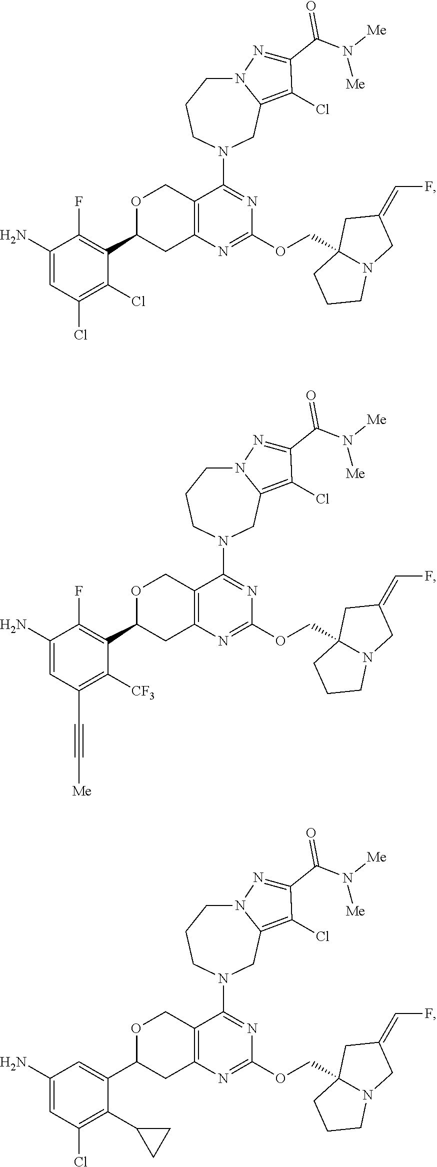 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1Cl)C3)CCC2 ; CC#Cc1cc(N)c(F)c([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C/C(=C\F)C4)nc3c1COC(c1cc(N)cc(Cl)c1C1CC1)C3)CCC2]

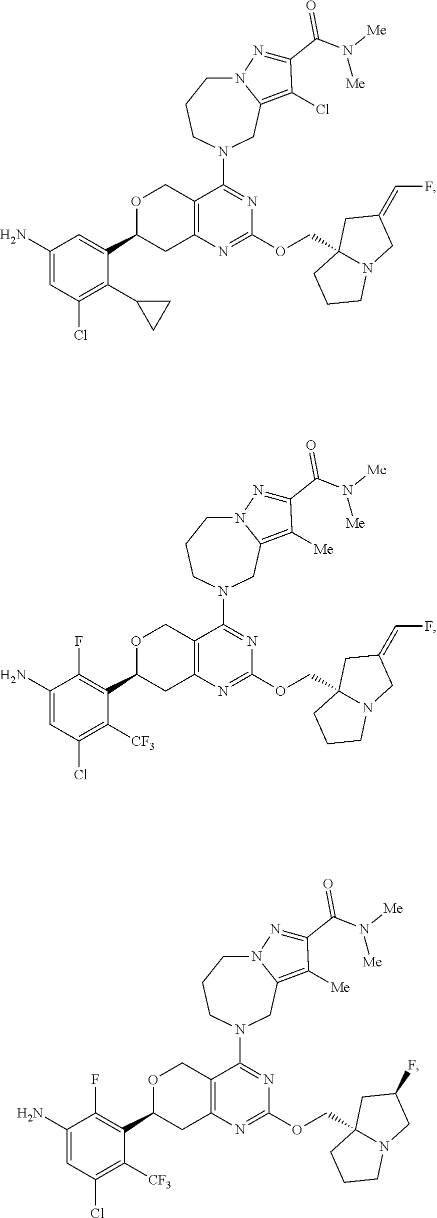 [SMILES: CN(C)C(=O)c1nn2c(c1Cl)CN(c1nc(OC[C@@]34CCCN3C/C(=C\F)C4)nc3c1CO[C@H](c1cc(N)cc(Cl)c1C1CC1)C3)CCC2 ; Cc1c(C(=O)N(C)C)nn2c1CN(c1nc(OC[C@@]34CCCN3C/C(=C\F)C4)nc3c1CO[C@H](c1c(F)c(N)cc(Cl)c1C(F)(F)F)C3)CCC2 ; Cc1c(C(=O)N(C)C)nn2c1CN(c1nc(OC[C@@]34CCCN3C[C@H](F)C4)nc3c1CO[C@H](c1c(F)c(N)cc(Cl)c1C(F)(F)F)C3)CCC2]

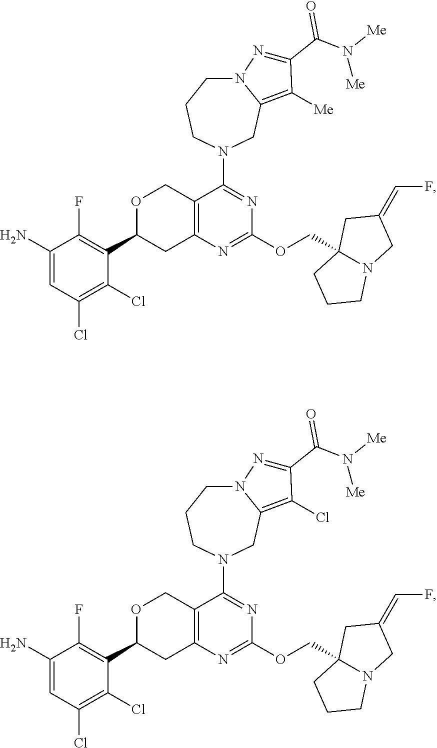 [SMILES: Cc1c(C(=O)N(C)C)nn2c1CN(c1nc(OC[C@@]34CCCN3C/C(=C\F)C4)nc3c1CO[C@H](c1c(F)c(N)cc(Cl)c1Cl)C3)CCC2 ; CN(C)C(=O)c1nn2c(c1Cl)CN(c1nc(OC[C@@]34CCCN3C/C(=C\F)C4)nc3c1CO[C@H](c1c(F)c(N)cc(Cl)c1Cl)C3)CCC2]

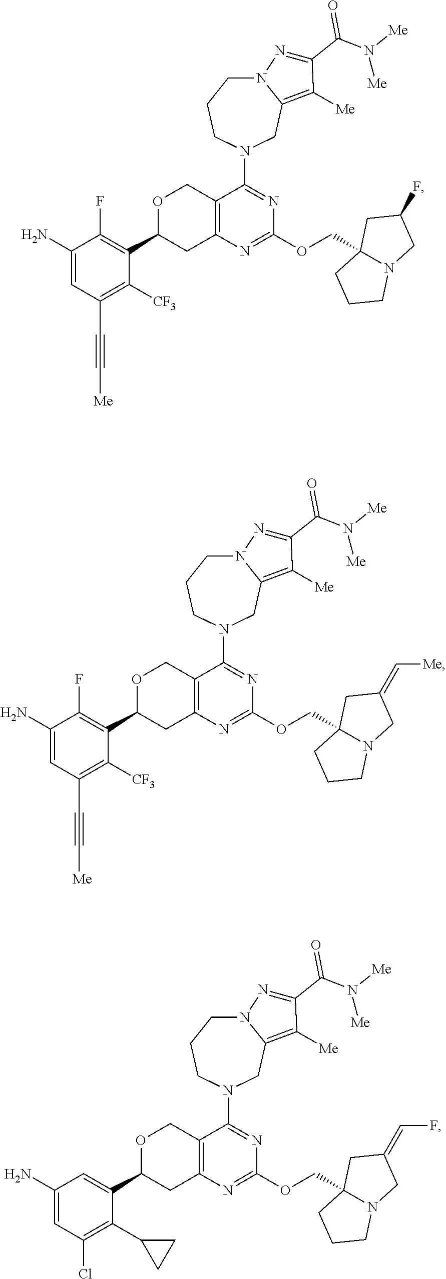 [SMILES: CC#Cc1cc(N)c(F)c([C@@H]2Cc3nc(OC[C@@]45CCCN4C[C@H](F)C5)nc(N4CCCn5nc(C(=O)N(C)C)c(C)c5C4)c3CO2)c1C(F)(F)F ; CC#Cc1cc(N)c(F)c([C@@H]2Cc3nc(OC[C@@]45CCCN4C/C(=C\C)C5)nc(N4CCCn5nc(C(=O)N(C)C)c(C)c5C4)c3CO2)c1C(F)(F)F ; Cc1c(C(=O)N(C)C)nn2c1CN(c1nc(OC[C@@]34CCCN3C/C(=C\F)C4)nc3c1CO[C@H](c1cc(N)cc(Cl)c1C1CC1)C3)CCC2]

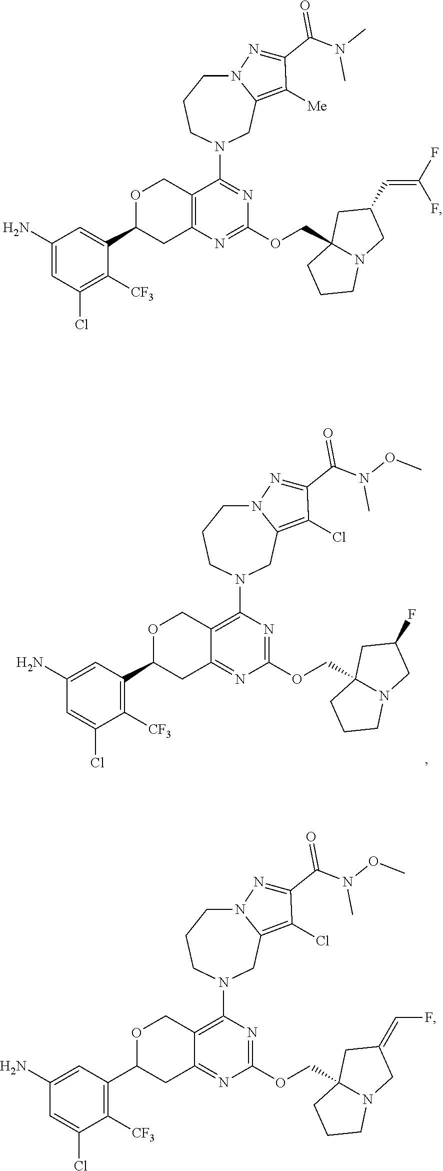 [SMILES: Cc1c(C(=O)N(C)C)nn2c1CN(c1nc(OC[C@]34CCCN3C[C@@H](C=C(F)F)C4)nc3c1CO[C@H](c1cc(N)cc(Cl)c1C(F)(F)F)C3)CCC2 ; CON(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2 ; CON(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2]

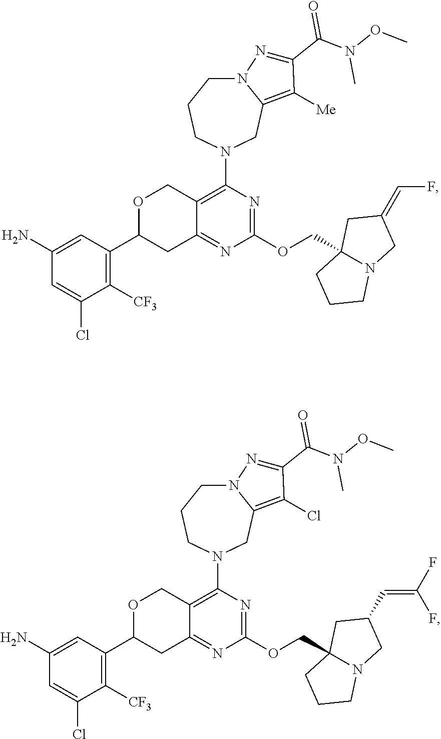 [SMILES: CON(C)C(=O)c1nn2c(c1C)CN(c1nc(OC[C@@]34CCCN3C/C(=C\F)C4)nc3c1COC(c1cc(N)cc(Cl)c1C(F)(F)F)C3)CCC2 ; CON(C)C(=O)c1nn2c(c1Cl)CN(c1nc(OC[C@]34CCCN3C[C@@H](C=C(F)F)C4)nc3c1COC(c1cc(N)cc(Cl)c1C(F)(F)F)C3)CCC2]

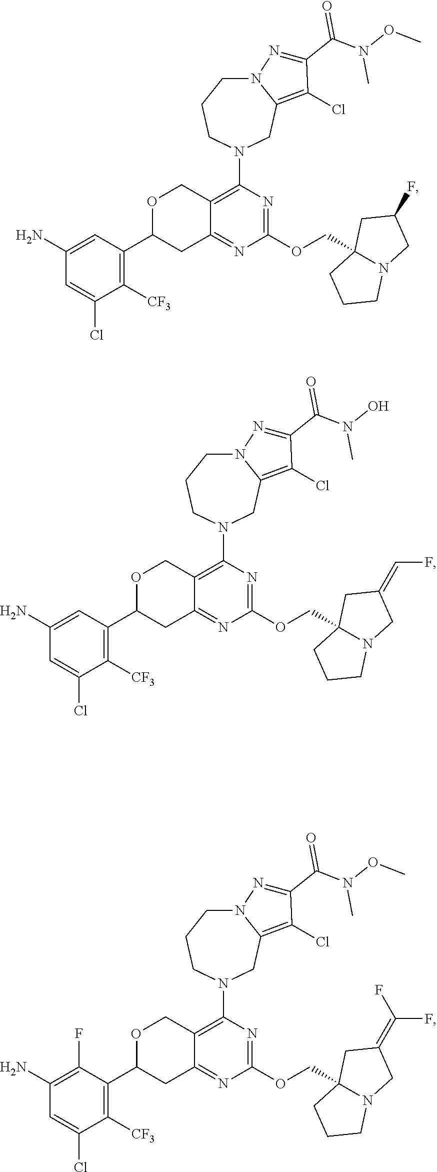 [SMILES: CON(C)C(=O)c1nn2c(c1Cl)CN(c1nc(OC[C@@]34CCCN3C[C@H](F)C4)nc3c1COC(c1cc(N)cc(Cl)c1C(F)(F)F)C3)CCC2 ; CN(O)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2 ; CON(C)C(=O)c1nn2c(c1Cl)CN(c1nc(OC[C@@]34CCCN3CC(=C(F)F)C4)nc3c1COC(c1c(F)c(N)cc(Cl)c1C(F)(F)F)C3)CCC2]

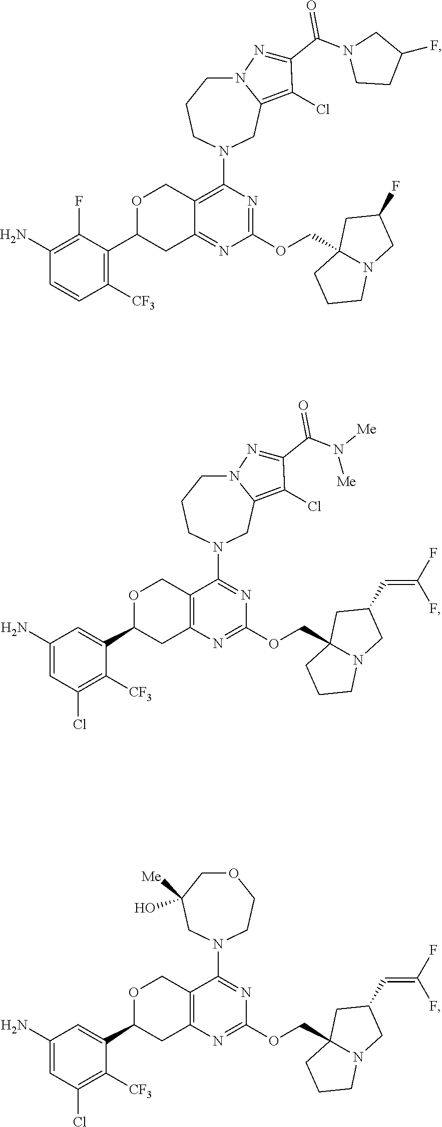 [SMILES: Nc1ccc(C(F)(F)F)c(C2Cc3nc(OC[C@@]45CCCN4C[C@H](F)C5)nc(N4CCCn5nc(C(=O)N6CCC(F)C6)c(Cl)c5C4)c3CO2)c1F ; CN(C)C(=O)c1nn2c(c1Cl)CN(c1nc(OC[C@]34CCCN3C[C@@H](C=C(F)F)C4)nc3c1CO[C@H](c1cc(N)cc(Cl)c1C(F)(F)F)C3)CCC2 ; C[C@@]1(O)COCCN(c2nc(OC[C@]34CCCN3C[C@@H](C=C(F)F)C4)nc3c2CO[C@H](c2cc(N)cc(Cl)c2C(F)(F)F)C3)C1]

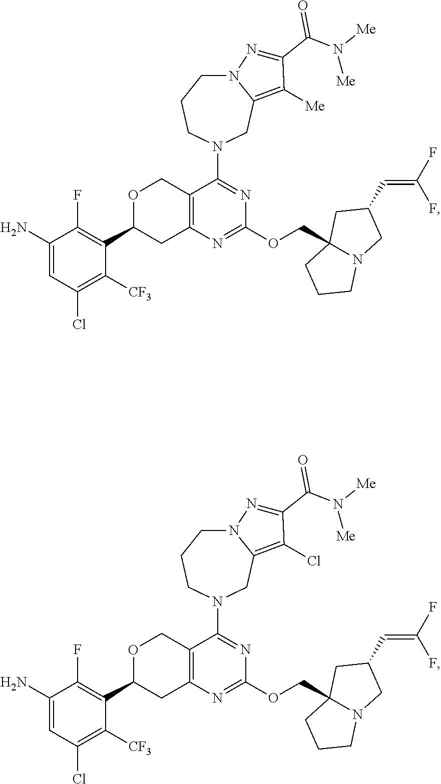 [SMILES: Cc1c(C(=O)N(C)C)nn2c1CN(c1nc(OC[C@]34CCCN3C[C@@H](C=C(F)F)C4)nc3c1CO[C@H](c1c(F)c(N)cc(Cl)c1C(F)(F)F)C3)CCC2 ; CN(C)C(=O)c1nn2c(c1Cl)CN(c1nc(OC[C@]34CCCN3C[C@@H](C=C(F)F)C4)nc3c1CO[C@H](c1c(F)c(N)cc(Cl)c1C(F)(F)F)C3)CCC2]

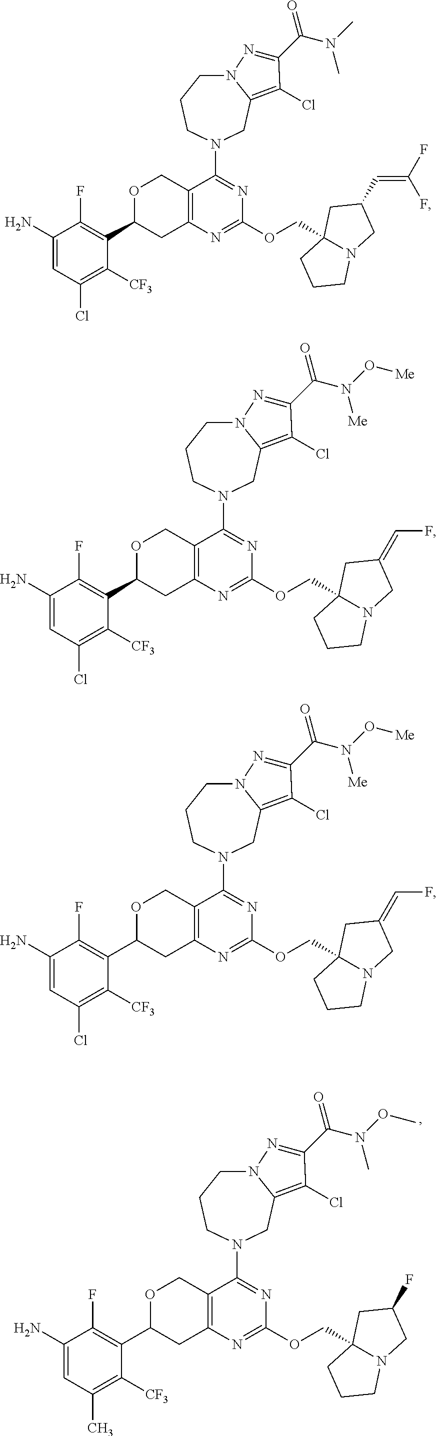 [SMILES: CN(C)C(=O)c1nn2c(c1Cl)CN(c1nc(OC[C@@]34CCCN3C[C@@H](C=C(F)F)C4)nc3c1CO[C@H](c1c(F)c(N)cc(Cl)c1C(F)(F)F)C3)CCC2 ; CON(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2 ; CON(C)C(=O)c1nn2c(c1Cl)CN(c1nc(OC[C@@]34CCCN3C/C(=C\F)C4)nc3c1COC(c1c(F)c(N)cc(Cl)c1C(F)(F)F)C3)CCC2 ; CON(C)C(=O)c1nn2c(c1Cl)CN(c1nc(OC[C@@]34CCCN3C[C@H](F)C4)nc3c1COC(c1c(F)c(N)cc(C)c1C(F)(F)F)C3)CCC2 ; ","]

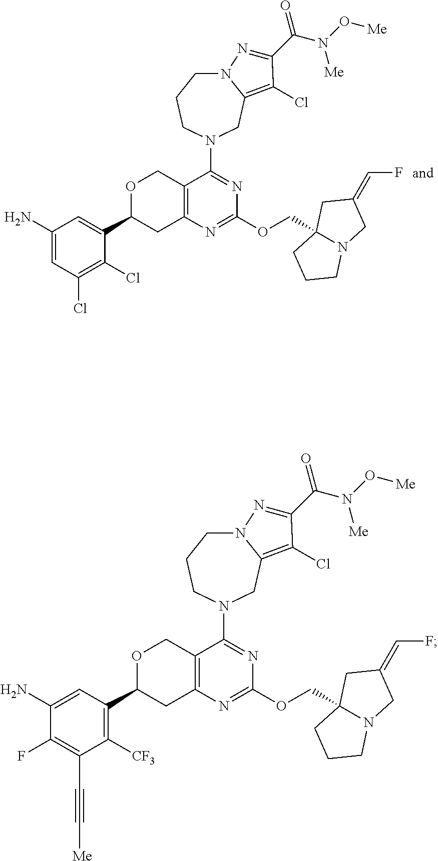 [SMILES: CON(C)C(=O)c1nn2c(c1Cl)CN(c1nc(OC[C@@]34CCCN3C/C(=C\F)C4)nc3c1CO[C@H](c1cc(N)cc(Cl)c1Cl)C3)CCC2 ; "and" ; CC#Cc1c(F)c(N)cc([C@@H]2Cc3nc(OC[C@@]45CCCN4C/C(=C\F)C5)nc(N4CCCn5nc(C(=O)N(C)OC)c(Cl)c5C4)c3CO2)c1C(F)(F)F]

or a stereoisomer, a tautomer, or a pharmaceutically acceptable salt thereof.

In some embodiments, the compound is selected from

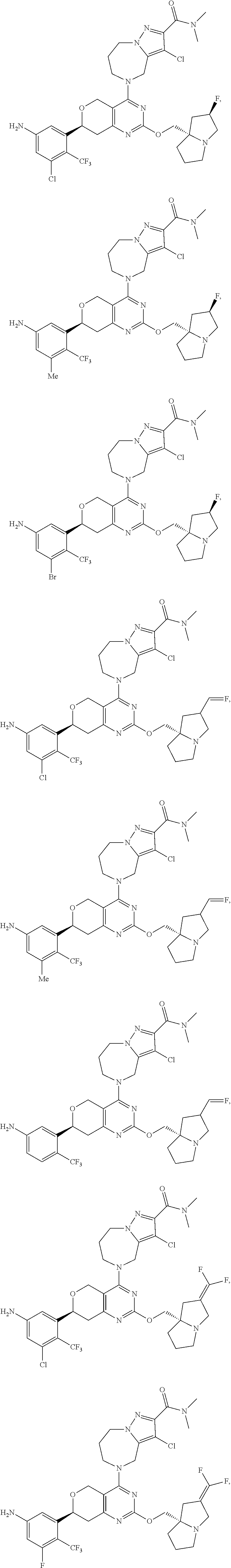

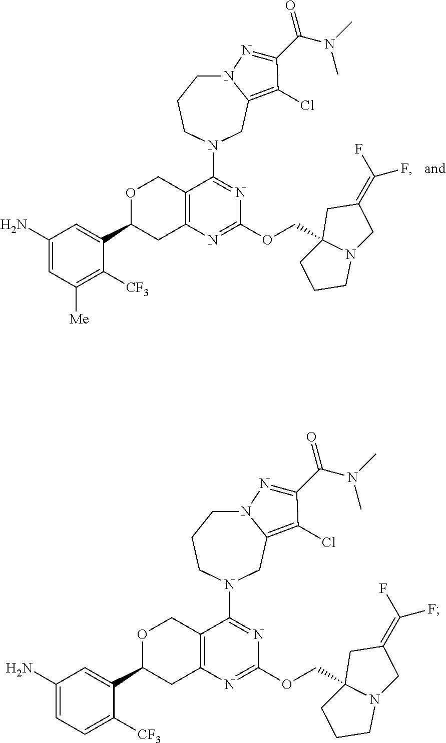 [SMILES: Cc1cc(N)cc([C@@H]2Cc3nc(OC[C@@]45CCCN4CC(=C(F)F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3CC(=C(F)F)C4)nc3c1CO[C@H](c1cc(N)ccc1C(F)(F)F)C3)CCC2]

or a stereoisomer, a tautomer, or a pharmaceutically acceptable salt thereof.

In some embodiments, the compound is selected from:

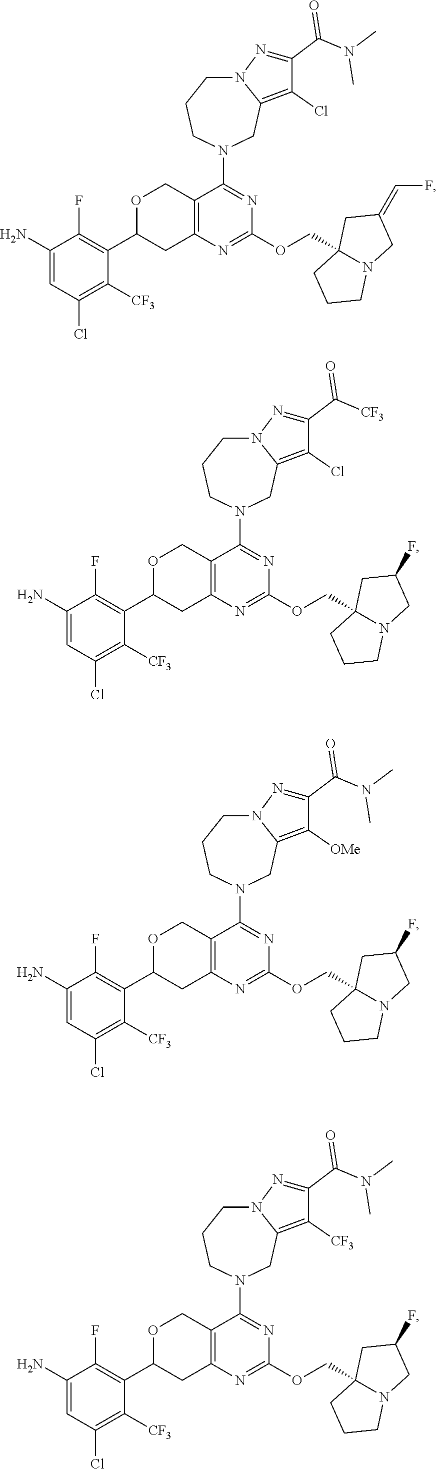 [SMILES: CN(C)C(=O)c1nn2c(c1Cl)CN(c1nc(OC[C@@]34CCCN3C/C(=C\F)C4)nc3c1COC(c1c(F)c(N)cc(Cl)c1C(F)(F)F)C3)CCC2 ; Nc1cc(Cl)c(C(F)(F)F)c(C2Cc3nc(OC[C@@]45CCCN4C[C@H](F)C5)nc(N4CCCn5nc(C(=O)C(F)(F)F)c(Cl)c5C4)c3CO2)c1F ; COc1c(C(=O)N(C)C)nn2c1CN(c1nc(OC[C@@]34CCCN3C[C@H](F)C4)nc3c1COC(c1c(F)c(N)cc(Cl)c1C(F)(F)F)C3)CCC2 ; CN(C)C(=O)c1nn2c(c1C(F)(F)F)CN(c1nc(OC[C@@]34CCCN3C[C@H](F)C4)nc3c1COC(c1c(F)c(N)cc(Cl)c1C(F)(F)F)C3)CCC2]

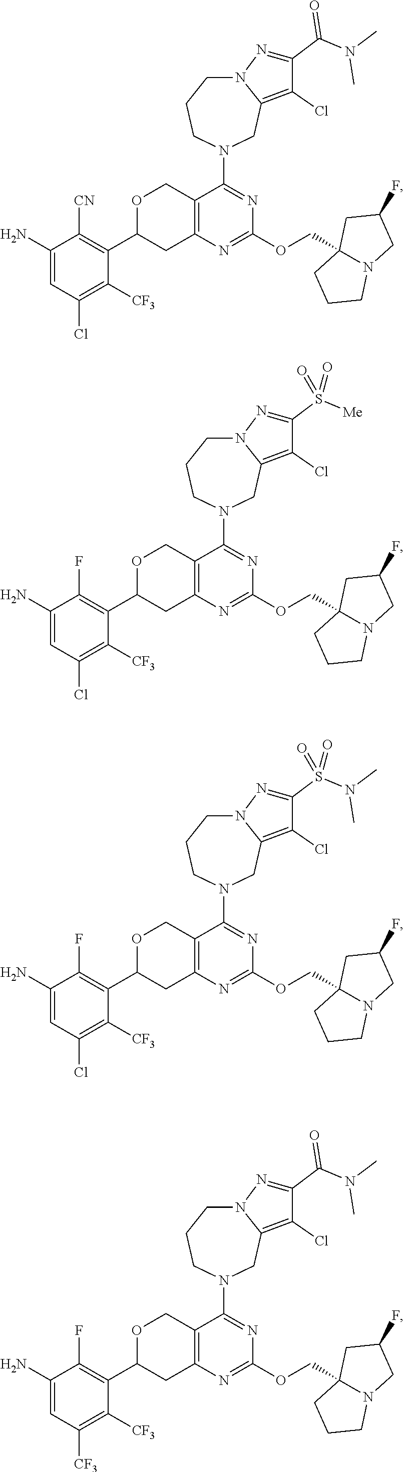 [SMILES: CN(C)C(=O)c1nn2c(c1Cl)CN(c1nc(OC[C@@]34CCCN3C[C@H](F)C4)nc3c1COC(c1c(C#N)c(N)cc(Cl)c1C(F)(F)F)C3)CCC2 ; CS(=O)(=O)c1nn2c(c1Cl)CN(c1nc(OC[C@@]34CCCN3C[C@H](F)C4)nc3c1COC(c1c(F)c(N)cc(Cl)c1C(F)(F)F)C3)CCC2 ; CN(C)S(=O)(=O)c1nn2c(c1Cl)CN(c1nc(OC[C@@]34CCCN3C[C@H](F)C4)nc3c1COC(c1c(F)c(N)cc(Cl)c1C(F)(F)F)C3)CCC2 ; CN(C)C(=O)c1nn2c(c1Cl)CN(c1nc(OC[C@@]34CCCN3C[C@H](F)C4)nc3c1COC(c1c(F)c(N)cc(C(F)(F)F)c1C(F)(F)F)C3)CCC2]

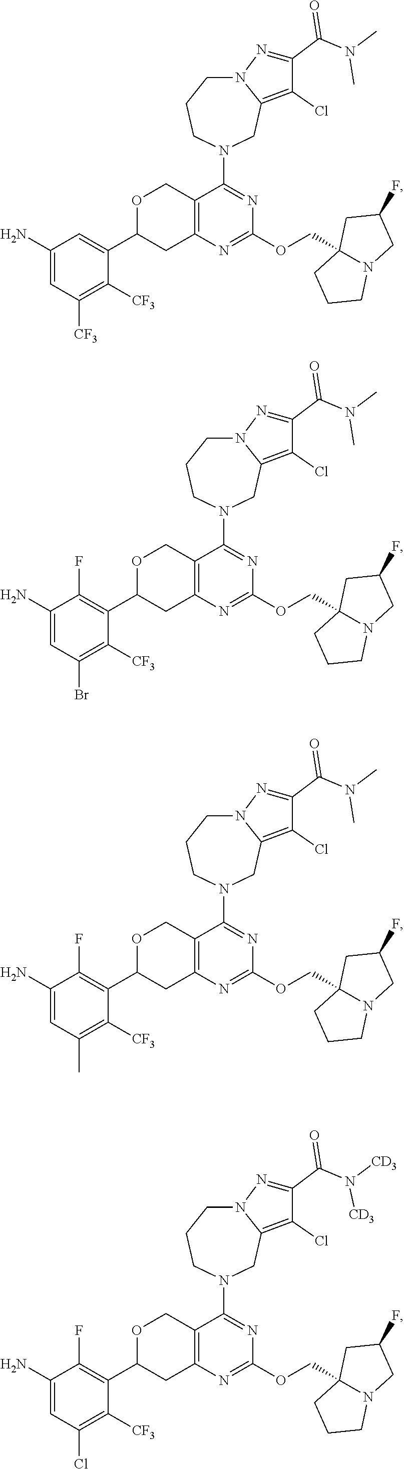 [SMILES: CN(C)C(=O)c1nn2c(c1Cl)CN(c1nc(OC[C@@]34CCCN3C[C@H](F)C4)nc3c1COC(c1cc(N)cc(C(F)(F)F)c1C(F)(F)F)C3)CCC2 ; CN(C)C(=O)c1nn2c(c1Cl)CN(c1nc(OC[C@@]34CCCN3C[C@H](F)C4)nc3c1COC(c1c(F)c(N)cc(Br)c1C(F)(F)F)C3)CCC2 ; Cc1cc(N)c(F)c(C2Cc3nc(OC[C@@]45CCCN4C[C@H](F)C5)nc(N4CCCn5nc(C(=O)N(C)C)c(Cl)c5C4)c3CO2)c1C(F)(F)F ; [2H]C([2H])([2H])N(C(=O)c1nn2c(c1Cl)CN(c1nc(OC[C@@]34CCCN3C[C@H](F)C4)nc3c1COC(c1c(F)c(N)cc(Cl)c1C(F)(F)F)C3)CCC2)C([2H])([2H])[2H]]

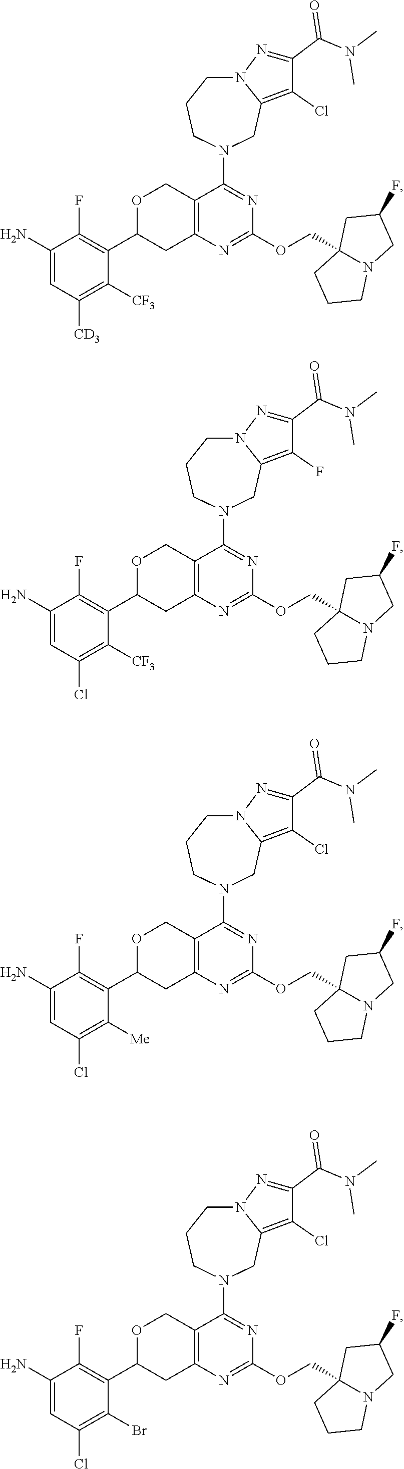 [SMILES: [2H]C([2H])([2H])c1cc(N)c(F)c(C2Cc3nc(OC[C@@]45CCCN4C[C@H](F)C5)nc(N4CCCn5nc(C(=O)N(C)C)c(Cl)c5C4)c3CO2)c1C(F)(F)F ; CN(C)C(=O)c1nn2c(c1F)CN(c1nc(OC[C@@]34CCCN3C[C@H](F)C4)nc3c1COC(c1c(F)c(N)cc(Cl)c1C(F)(F)F)C3)CCC2 ; Cc1c(Cl)cc(N)c(F)c1C1Cc2nc(OC[C@@]34CCCN3C[C@H](F)C4)nc(N3CCCn4nc(C(=O)N(C)C)c(Cl)c4C3)c2CO1 ; CN(C)C(=O)c1nn2c(c1Cl)CN(c1nc(OC[C@@]34CCCN3C[C@H](F)C4)nc3c1COC(c1c(F)c(N)cc(Cl)c1Br)C3)CCC2]

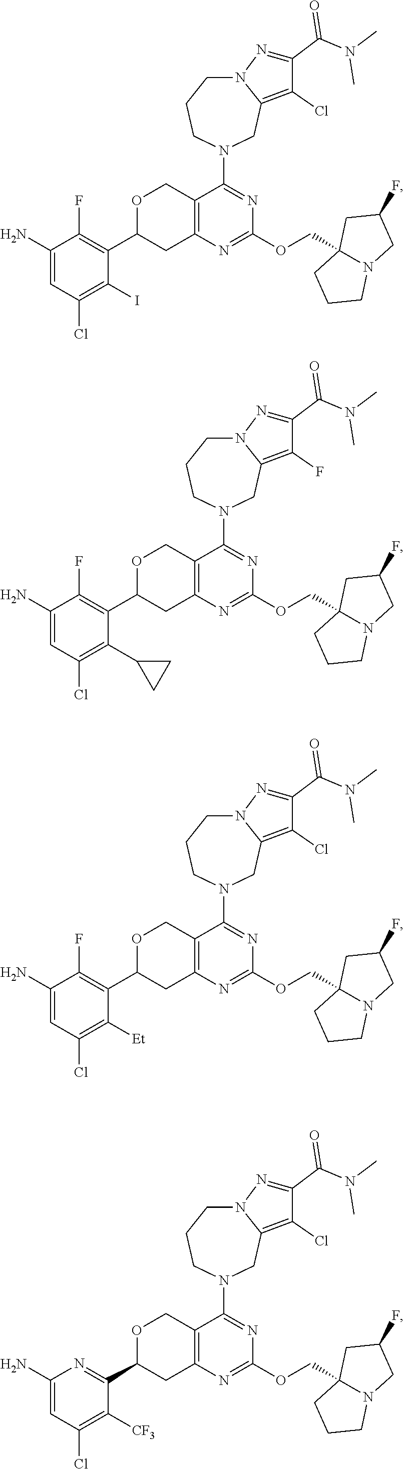 [SMILES: CN(C)C(=O)c1nn2c(c1Cl)CN(c1nc(OC[C@@]34CCCN3C[C@H](F)C4)nc3c1COC(c1c(F)c(N)cc(Cl)c1I)C3)CCC2 ; CN(C)C(=O)c1nn2c(c1F)CN(c1nc(OC[C@@]34CCCN3C[C@H](F)C4)nc3c1COC(c1c(F)c(N)cc(Cl)c1C1CC1)C3)CCC2 ; CCc1c(Cl)cc(N)c(F)c1C1Cc2nc(OC[C@@]34CCCN3C[C@H](F)C4)nc(N3CCCn4nc(C(=O)N(C)C)c(Cl)c4C3)c2CO1 ; CN(C)C(=O)c1nn2c(c1Cl)CN(c1nc(OC[C@@]34CCCN3C[C@H](F)C4)nc3c1CO[C@H](c1nc(N)cc(Cl)c1C(F)(F)F)C3)CCC2]

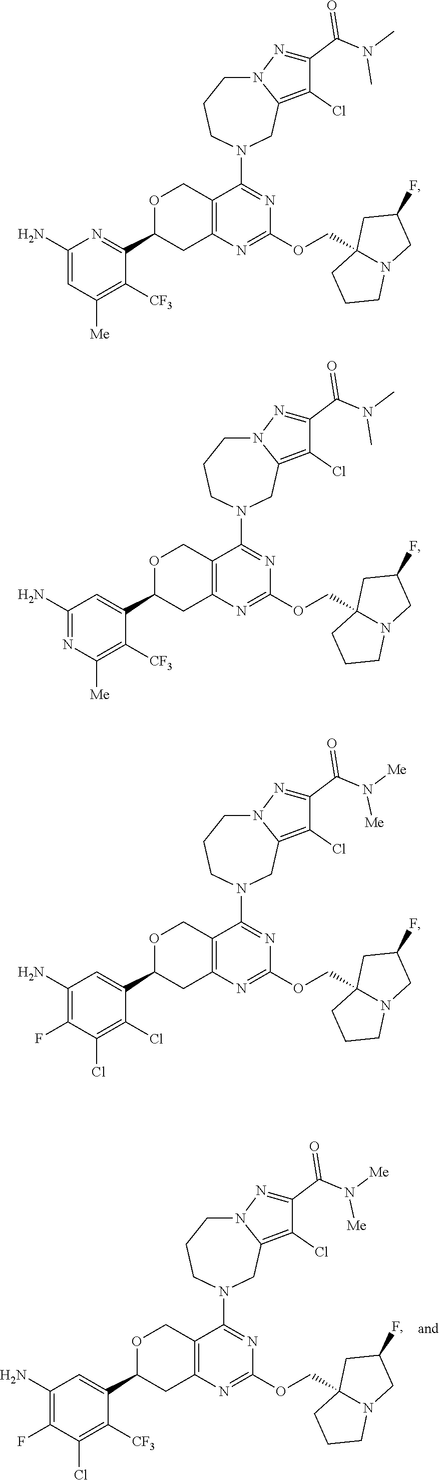 [SMILES: Cc1cc(N)nc([C@@H]2Cc3nc(OC[C@@]45CCCN4C[C@H](F)C5)nc(N4CCCn5nc(C(=O)N(C)C)c(Cl)c5C4)c3CO2)c1C(F)(F)F ; Cc1nc(N)cc([C@@H]2Cc3nc(OC[C@@]45CCCN4C[C@H](F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C[C@H](F)C4)nc3c1CO[C@H](c1cc(N)c(F)c(Cl)c1Cl)C3)CCC2 ; CN(C)C(=O)c1nn2c(c1Cl)CN(c1nc(OC[C@@]34CCCN3C[C@H](F)C4)nc3c1CO[C@H](c1cc(N)c(F)c(Cl)c1C(F)(F)F)C3)CCC2 ; "and"]

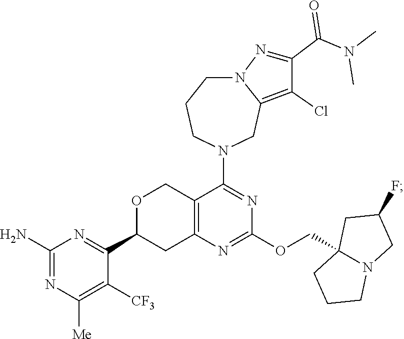 [SMILES: Cc1nc(N)nc([C@@H]2Cc3nc(OC[C@@]45CCCN4C[C@H](F)C5)nc(N4CCCn5nc(C(=O)N(C)C)c(Cl)c5C4)c3CO2)c1C(F)(F)F]

or a stereoisomer, a tautomer, or a pharmaceutically acceptable salt thereof.

In some embodiments, the compound is selected from

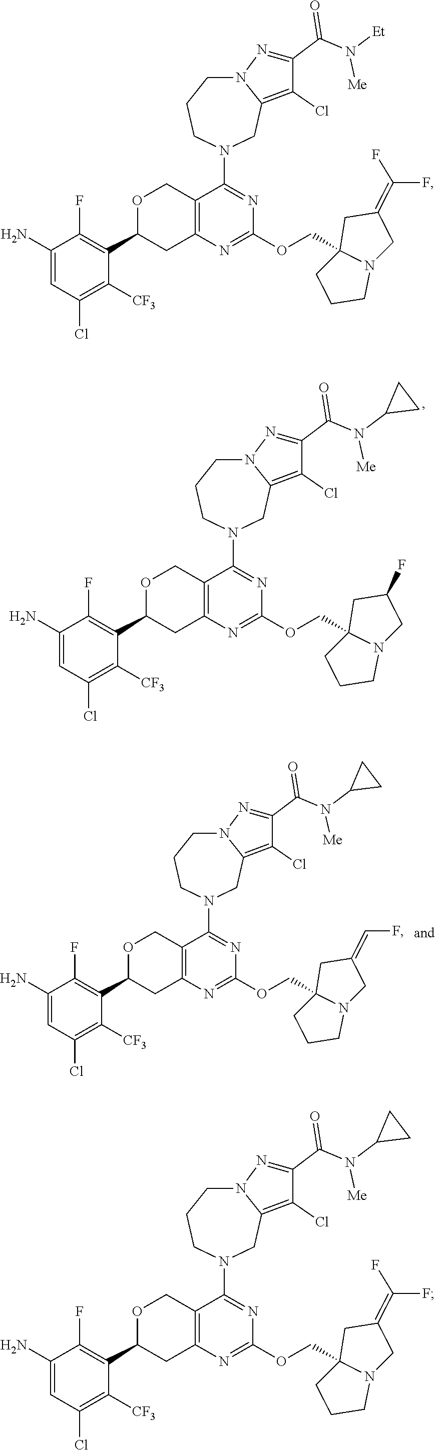 [SMILES: CCN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2 ; CN(C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2)C1CC1 ; CN(C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2)C1CC1 ; CN(C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2)C1CC1]

or a stereoisomer, a tautomer, or a pharmaceutically acceptable salt thereof.

In some embodiments, the compound is selected from

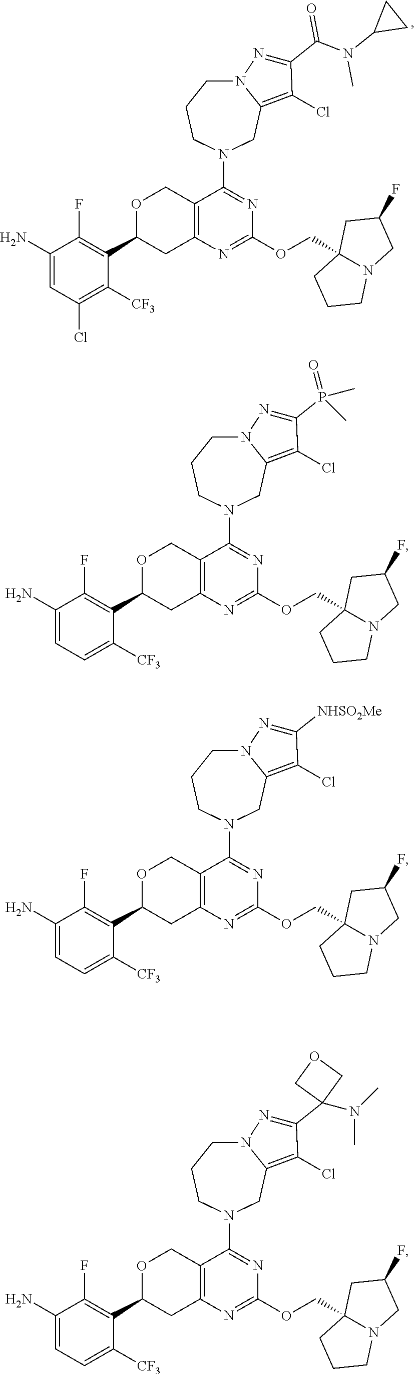

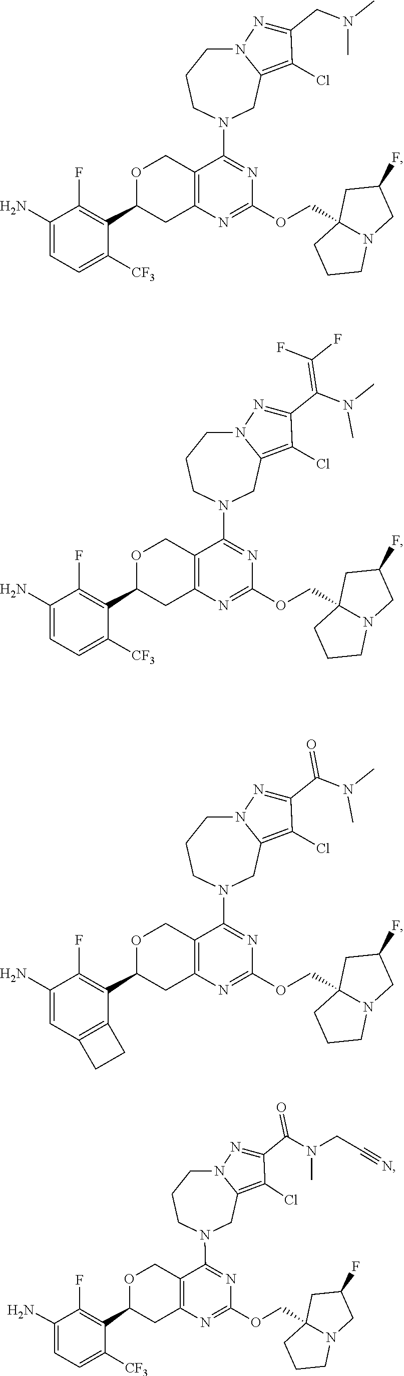 [SMILES: CN(C)Cc1nn2c(c1Cl)CN(c1nc(OC[C@@]34CCCN3C[C@H](F)C4)nc3c1CO[C@H](c1c(C(F)(F)F)ccc(N)c1F)C3)CCC2 ; CN(C)C(=C(F)F)c1nn2c(c1Cl)CN(c1nc(OC[C@@]34CCCN3C[C@H](F)C4)nc3c1CO[C@H](c1c(C(F)(F)F)ccc(N)c1F)C3)CCC2 ; CN(C)C(=O)c1nn2c(c1Cl)CN(c1nc(OC[C@@]34CCCN3C[C@H](F)C4)nc3c1CO[C@H](c1c(F)c(N)cc4c1CC4)C3)CCC2 ; CN(CC#N)C(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2]

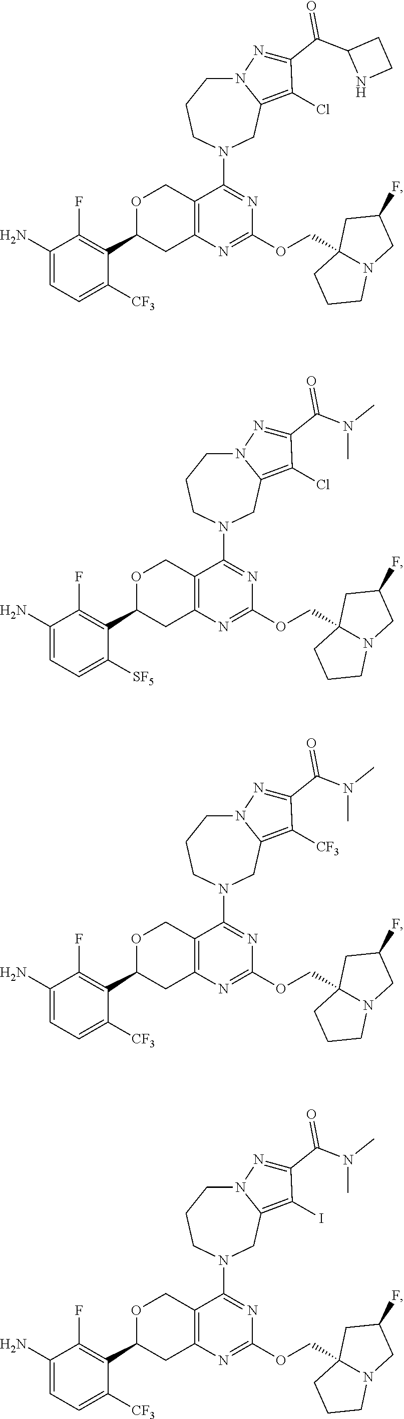

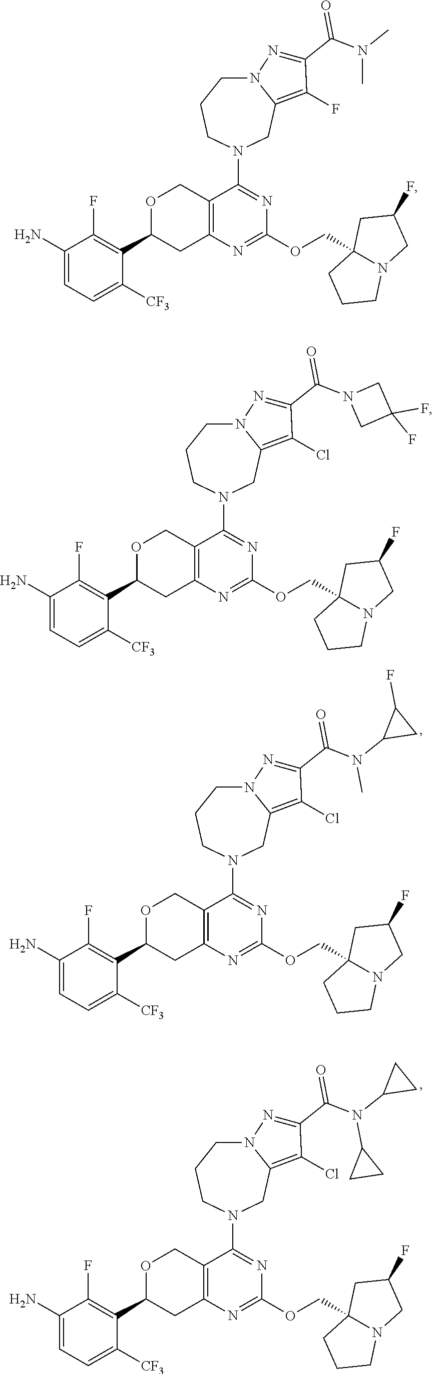

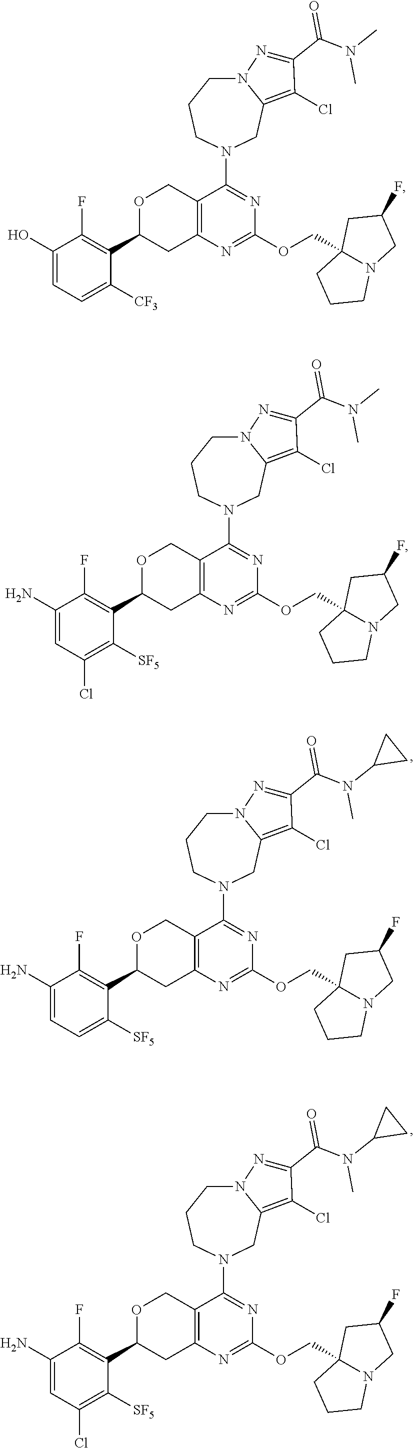

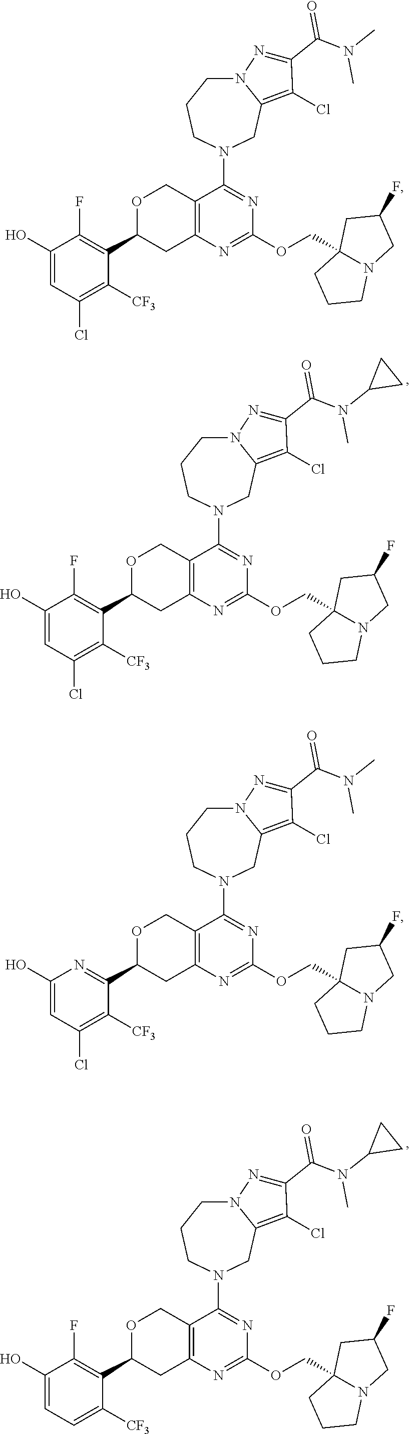 [SMILES: CN(C)C(=O)c1nn2c(c1Cl)CN(c1nc(OC[C@@]34CCCN3C[C@H](F)C4)nc3c1CO[C@H](c1c(F)c(O)cc(Cl)c1C(F)(F)F)C3)CCC2 ; CN(C(=O)c1nn2c(c1Cl)CN(c1nc(OC[C@@]34CCCN3C[C@H](F)C4)nc3c1CO[C@H](c1c(F)c(O)cc(Cl)c1C(F)(F)F)C3)CCC2)C1CC1 ; CN(C)C(=O)c1nn2c(c1Cl)CN(c1nc(OC[C@@]34CCCN3C[C@H](F)C4)nc3c1CO[C@H](c1nc(O)cc(Cl)c1C(F)(F)F)C3)CCC2 ; CN(C(=O)c1nn2c(c1Cl)CN(c1nc(OC[C@@]34CCCN3C[C@H](F)C4)nc3c1CO[C@H](c1c(C(F)(F)F)ccc(O)c1F)C3)CCC2)C1CC1]

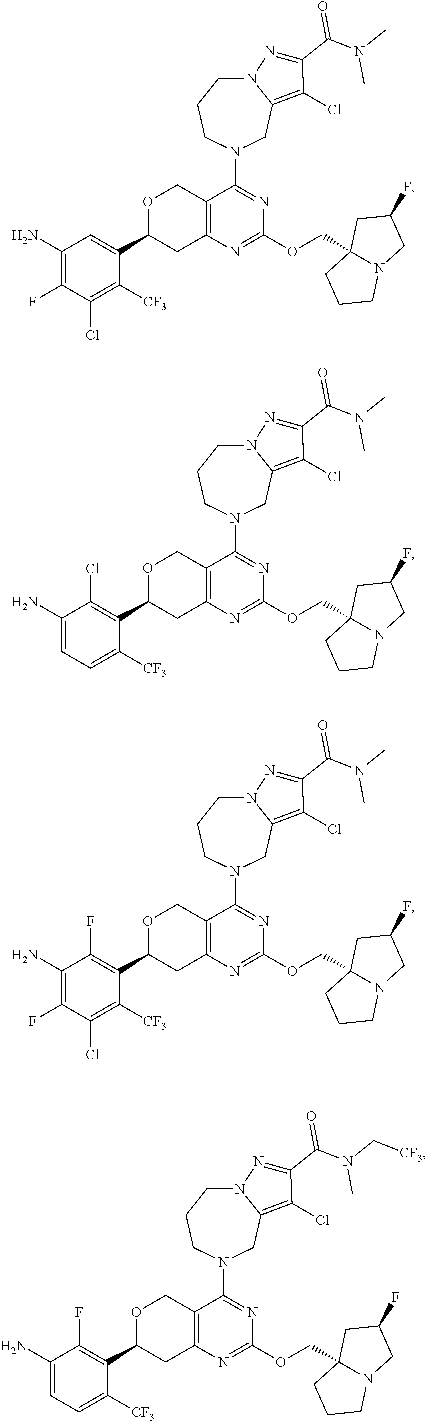 [SMILES: CN(C)C(=O)c1nn2c(c1Cl)CN(c1nc(OC[C@@]34CCCN3C[C@H](F)C4)nc3c1CO[C@H](c1cc(N)c(F)c(Cl)c1C(F)(F)F)C3)CCC2 ; CN(C)C(=O)c1nn2c(c1Cl)CN(c1nc(OC[C@@]34CCCN3C[C@H](F)C4)nc3c1CO[C@H](c1c(C(F)(F)F)ccc(N)c1Cl)C3)CCC2 ; CN(C)C(=O)c1nn2c(c1Cl)CN(c1nc(OC[C@@]34CCCN3C[C@H](F)C4)nc3c1CO[C@H](c1c(F)c(N)c(F)c(Cl)c1C(F)(F)F)C3)CCC2 ; CN(CC(F)(F)F)C(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2]

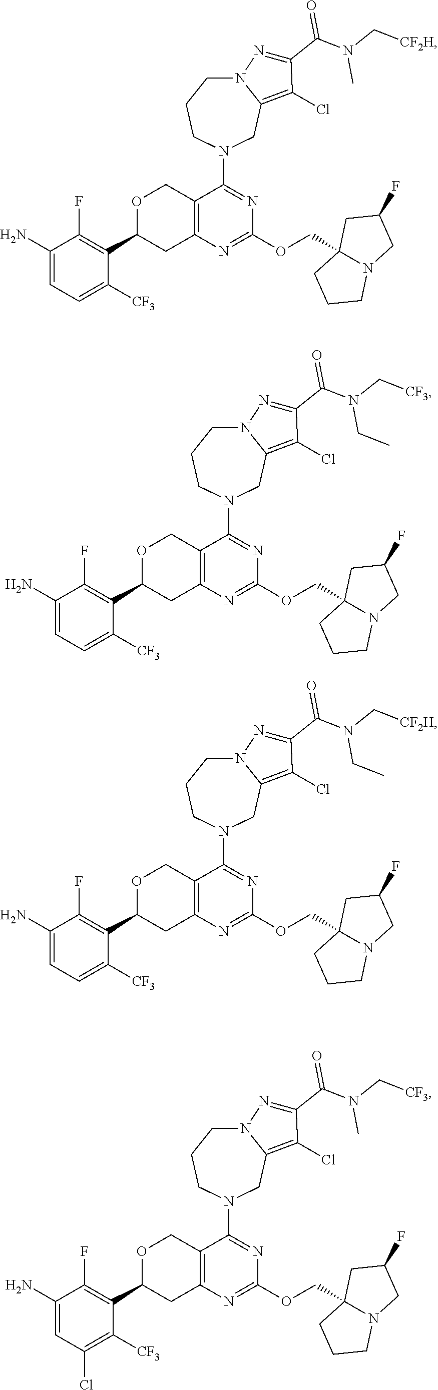

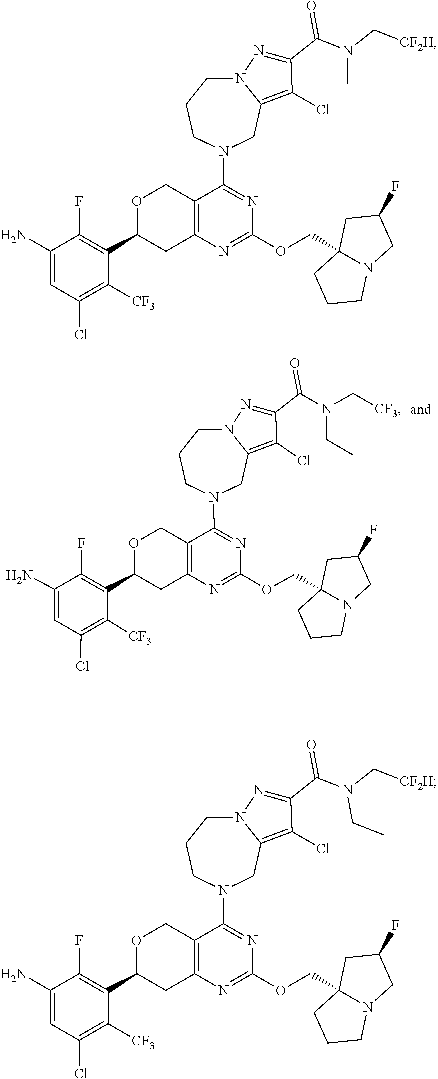 [SMILES: CN(CC(F)F)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2 ; CCN(CC(F)(F)F)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2 ; CCN(CC(F)F)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2]

or a stereoisomer, a tautomer, or a pharmaceutically acceptable salt thereof.

In some embodiments, the compound is selected from

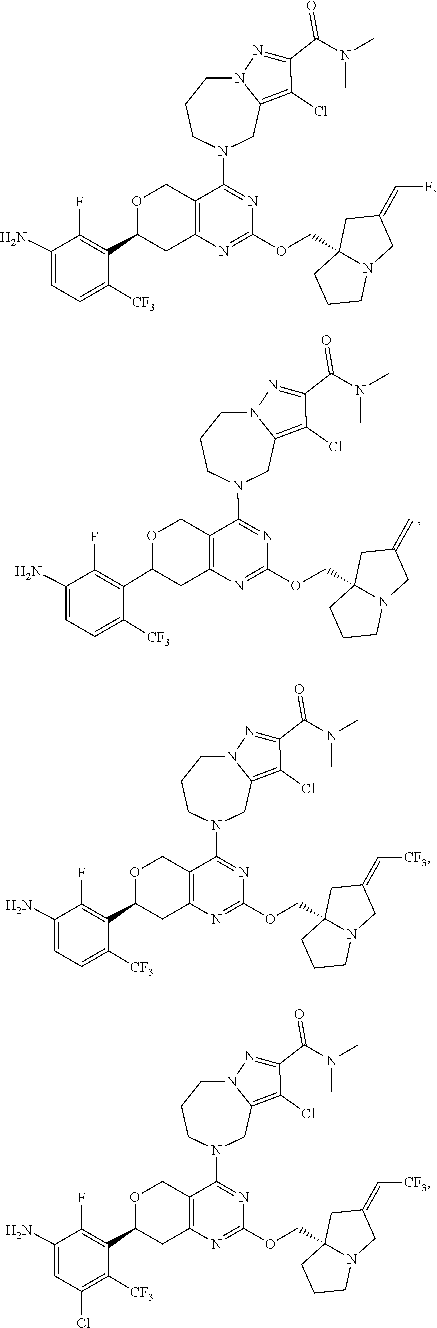

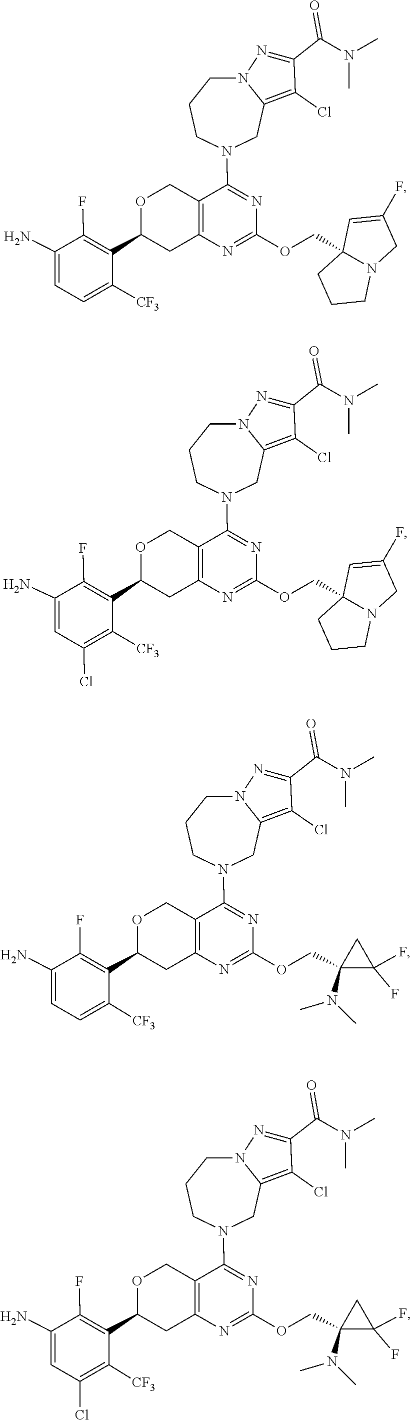 [SMILES: CN(C)C(=O)c1nn2c(c1Cl)CN(c1nc(OC[C@]34C=C(F)CN3CCC4)nc3c1CO[C@H](c1c(C(F)(F)F)ccc(N)c1F)C3)CCC2 ; CN(C)C(=O)c1nn2c(c1Cl)CN(c1nc(OC[C@]34C=C(F)CN3CCC4)nc3c1CO[C@H](c1c(F)c(N)cc(Cl)c1C(F)(F)F)C3)CCC2 ; CN(C)C(=O)c1nn2c(c1Cl)CN(c1nc(OC[C@]3(N(C)C)CC3(F)F)nc3c1CO[C@H](c1c(C(F)(F)F)ccc(N)c1F)C3)CCC2 ; CN(C)C(=O)c1nn2c(c1Cl)CN(c1nc(OC[C@]3(N(C)C)CC3(F)F)nc3c1CO[C@H](c1c(F)c(N)cc(Cl)c1C(F)(F)F)C3)CCC2]

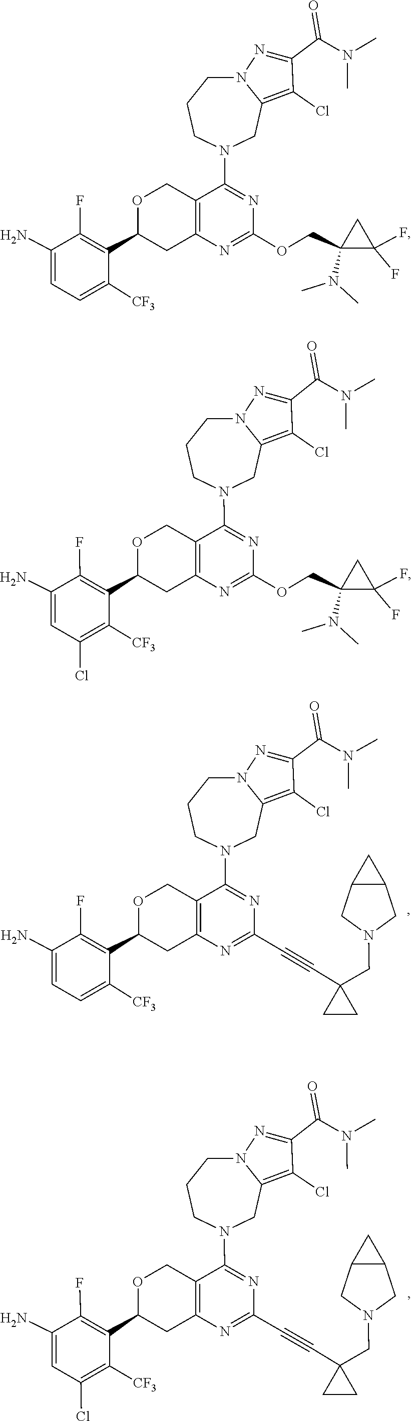 [SMILES: CN(C)C(=O)c1nn2c(c1Cl)CN(c1nc(OC[C@@]3(N(C)C)CC3(F)F)nc3c1CO[C@H](c1c(C(F)(F)F)ccc(N)c1F)C3)CCC2 ; CN(C)C(=O)c1nn2c(c1Cl)CN(c1nc(OC[C@@]3(N(C)C)CC3(F)F)nc3c1CO[C@H](c1c(F)c(N)cc(Cl)c1C(F)(F)F)C3)CCC2 ; CN(C)C(=O)c1nn2c(c1Cl)CN(c1nc(C#CC3(CN4CC5CC5C4)CC3)nc3c1CO[C@H](c1c(C(F)(F)F)ccc(N)c1F)C3)CCC2 ; CN(C)C(=O)c1nn2c(c1Cl)CN(c1nc(C#CC3(CN4CC5CC5C4)CC3)nc3c1CO[C@H](c1c(F)c(N)cc(Cl)c1C(F)(F)F)C3)CCC2]

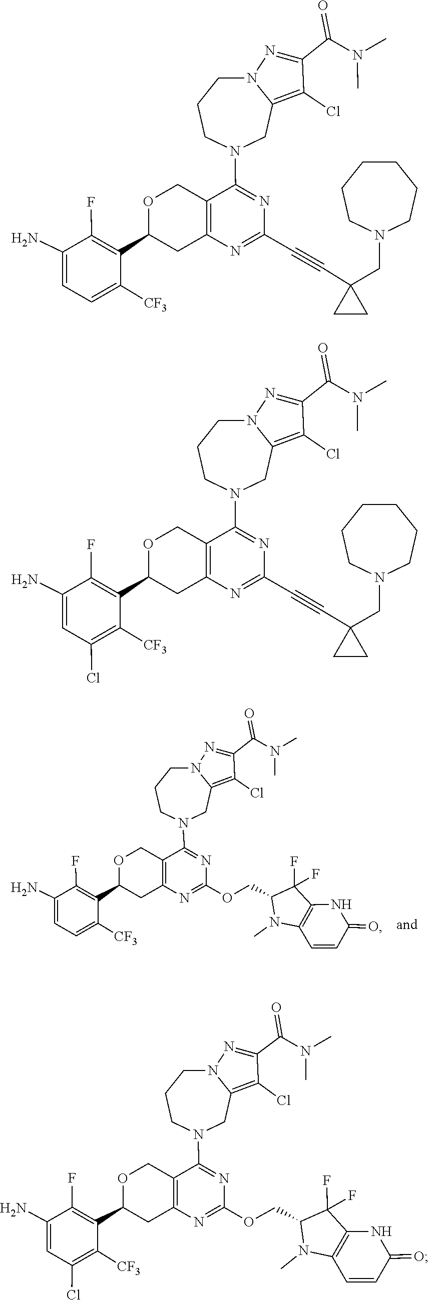

or a stereoisomer, a tautomer, or a pharmaceutically acceptable salt thereof.

In some embodiments, the compound is selected from

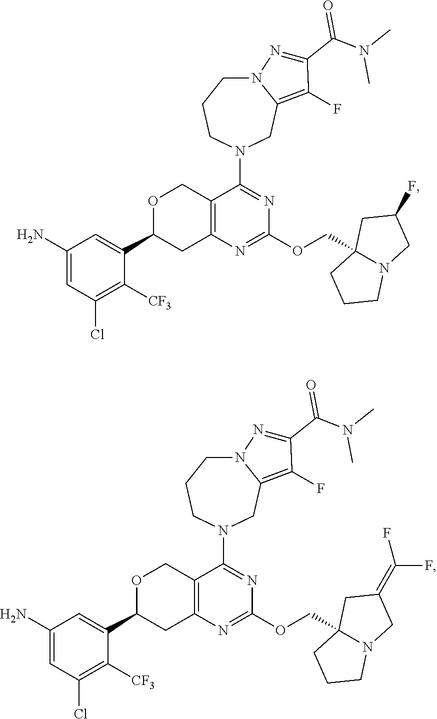 [SMILES: CN(C)C(=O)c1nn2c(c1F)CN(c1nc(OC[C@@]34CCCN3C[C@H](F)C4)nc3c1CO[C@H](c1cc(N)cc(Cl)c1C(F)(F)F)C3)CCC2 ; CN(C)C(=O)c1nn2c(c1F)CN(c1nc(OC[C@@]34CCCN3CC(=C(F)F)C4)nc3c1CO[C@H](c1cc(N)cc(Cl)c1C(F)(F)F)C3)CCC2]

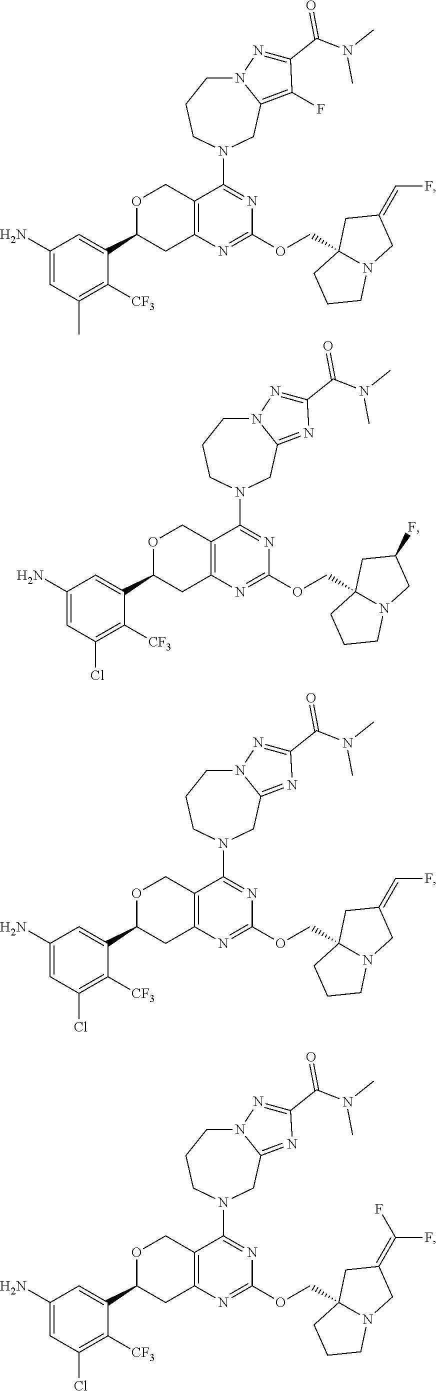

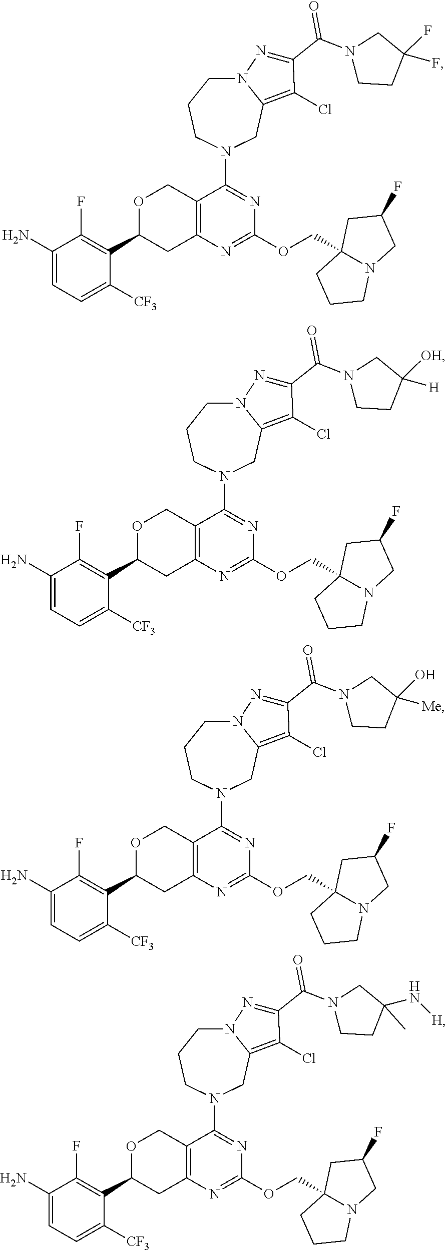

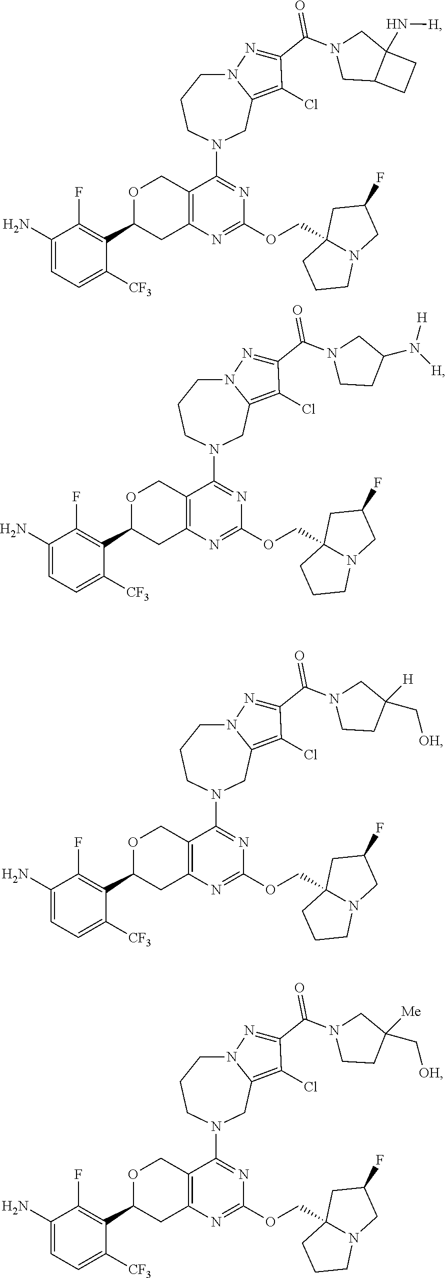

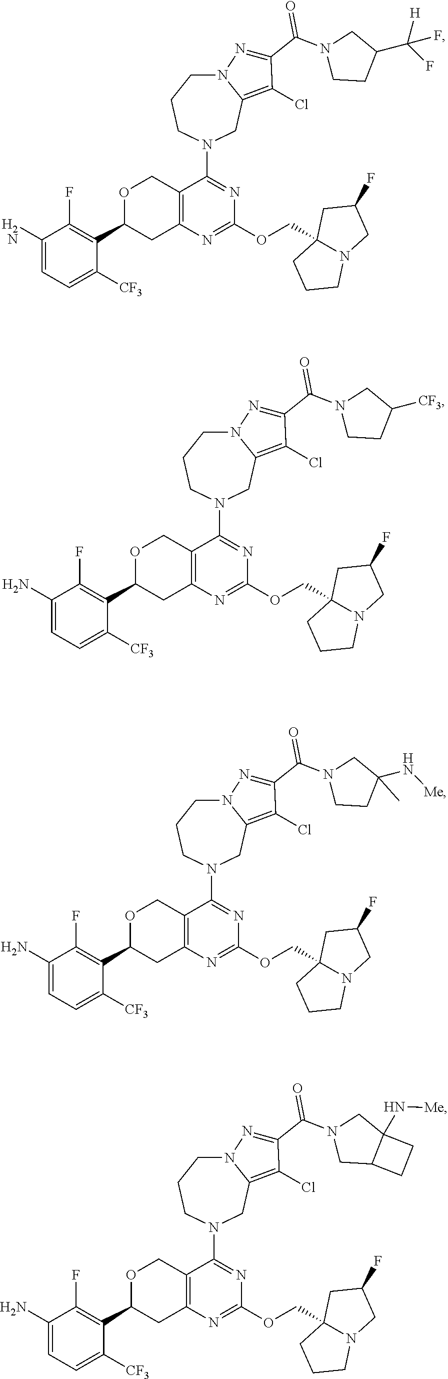 [SMILES: Nc1ccc(C(F)(F)F)c([C@@H]2Cc3nc(OC[C@@]45CCCN4C[C@H](F)C5)nc(N4CCCn5nc(C(=O)N6CCC(C(F)F)C6)c(Cl)c5C4)c3CO2)c1F ; Nc1ccc(C(F)(F)F)c([C@@H]2Cc3nc(OC[C@@]45CCCN4C[C@H](F)C5)nc(N4CCCn5nc(C(=O)N6CCC(C(F)(F)F)C6)c(Cl)c5C4)c3CO2)c1F ; CNC1(C)CCN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1 ; CNC12CCC1CN(C(=O)c1nn3c(c1Cl)CN(c1nc(OC[C@@]45CCCN4C[C@H](F)C5)nc4c1CO[C@H](c1c(C(F)(F)F)ccc(N)c1F)C4)CCC3)C2]

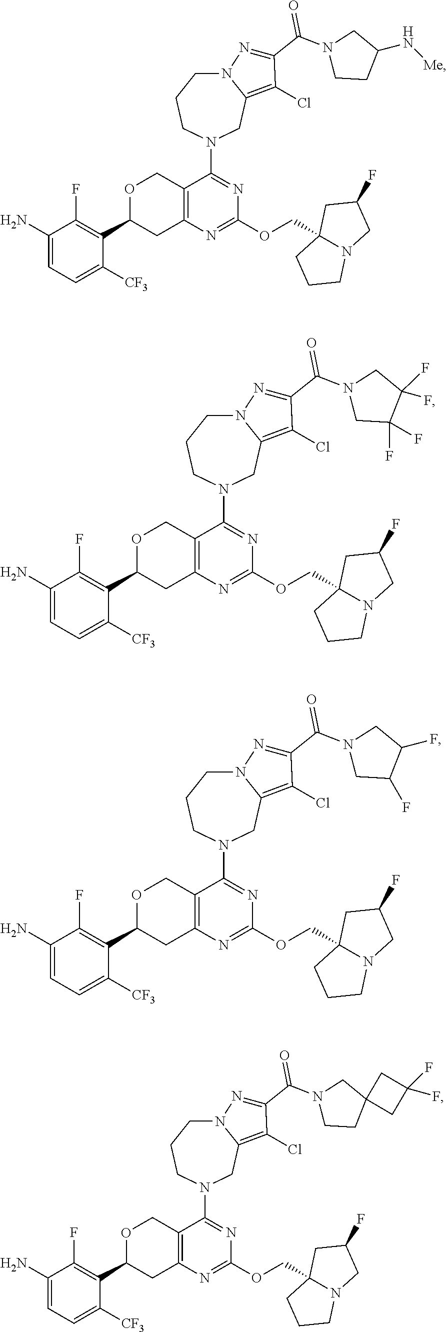 [SMILES: CNC1CCN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1 ; Nc1ccc(C(F)(F)F)c([C@@H]2Cc3nc(OC[C@@]45CCCN4C[C@H](F)C5)nc(N4CCCn5nc(C(=O)N6CC(F)(F)C(F)(F)C6)c(Cl)c5C4)c3CO2)c1F ; Nc1ccc(C(F)(F)F)c([C@@H]2Cc3nc(OC[C@@]45CCCN4C[C@H](F)C5)nc(N4CCCn5nc(C(=O)N6CC(F)C(F)C6)c(Cl)c5C4)c3CO2)c1F ; Nc1ccc(C(F)(F)F)c([C@@H]2Cc3nc(OC[C@@]45CCCN4C[C@H](F)C5)nc(N4CCCn5nc(C(=O)N6CCC7(C6)CC(F)(F)C7)c(Cl)c5C4)c3CO2)c1F]

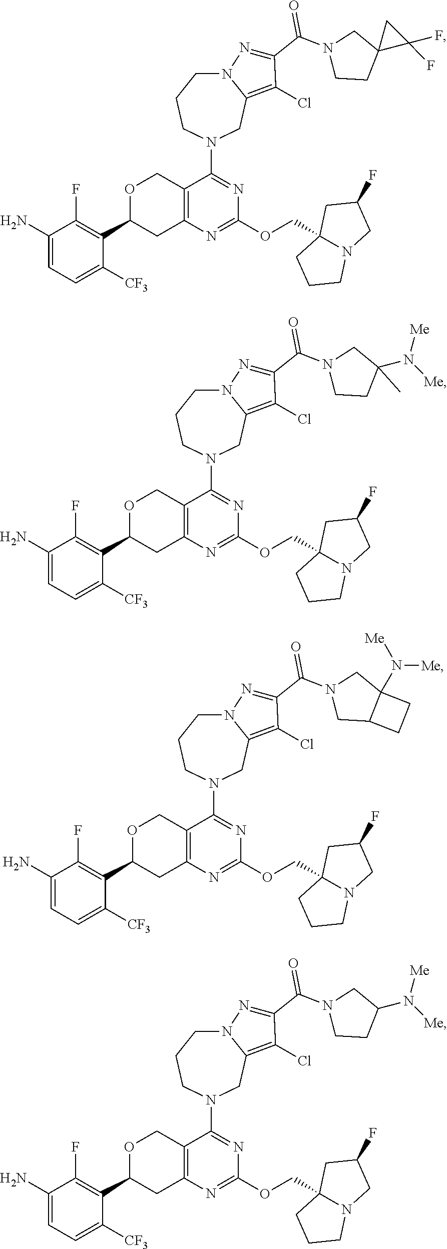

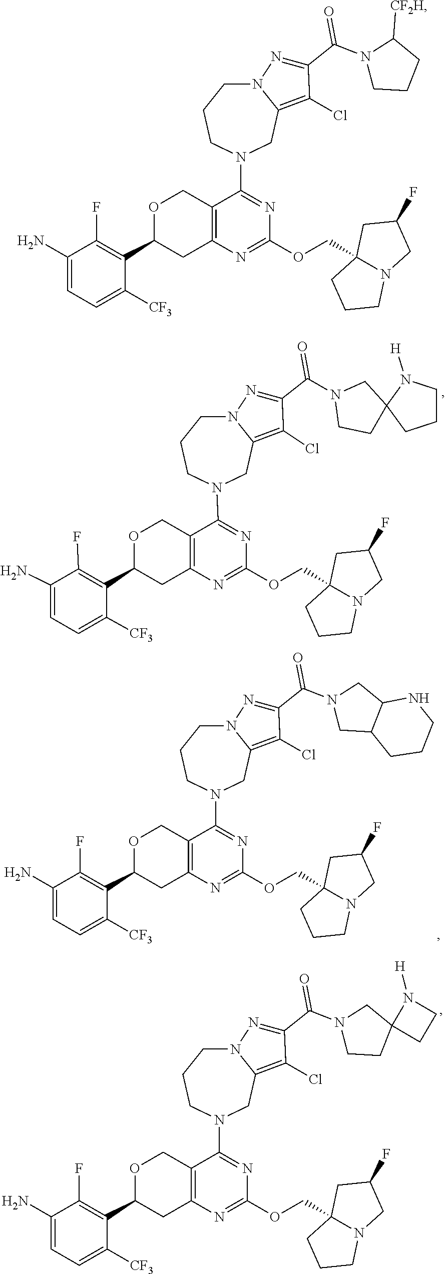

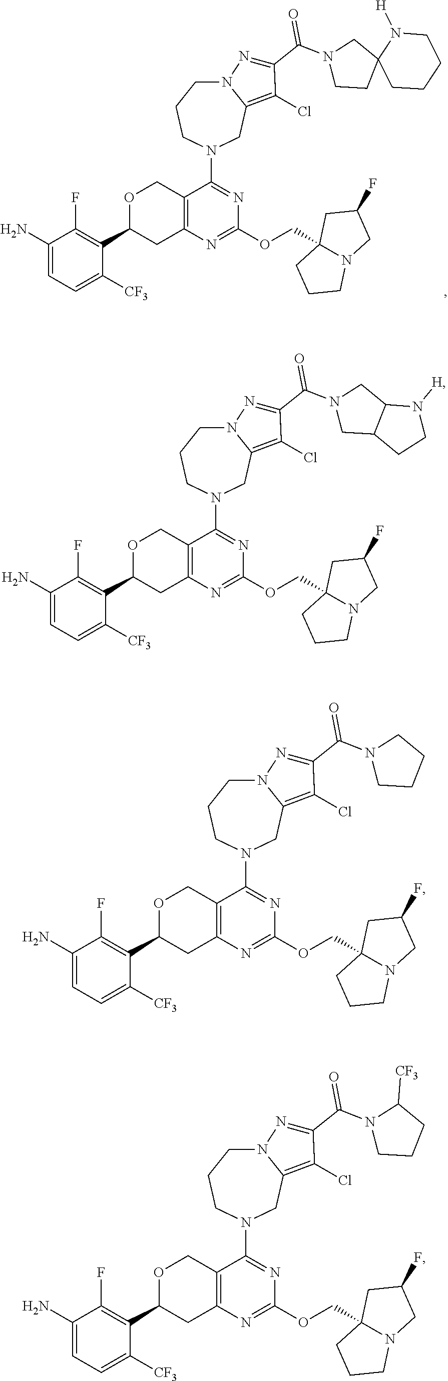

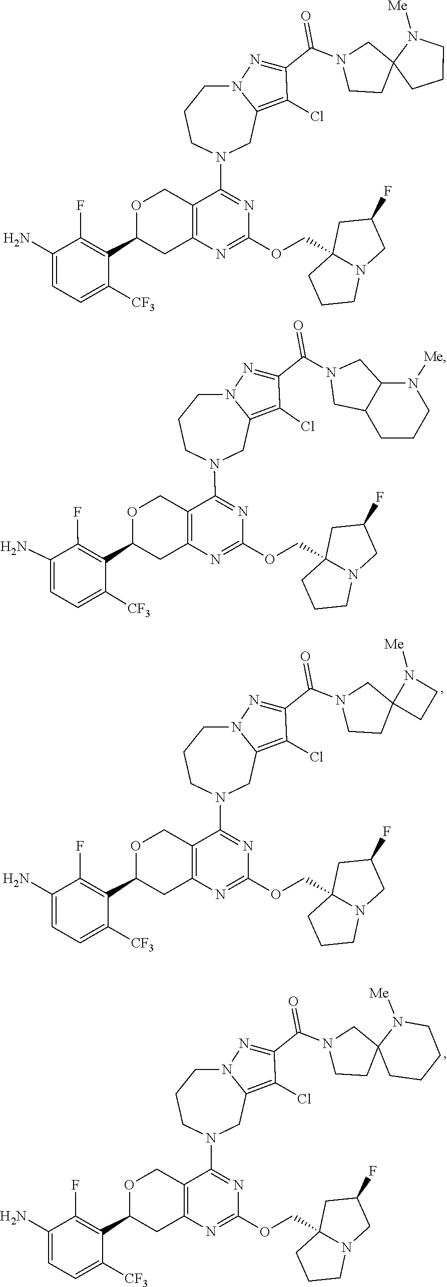

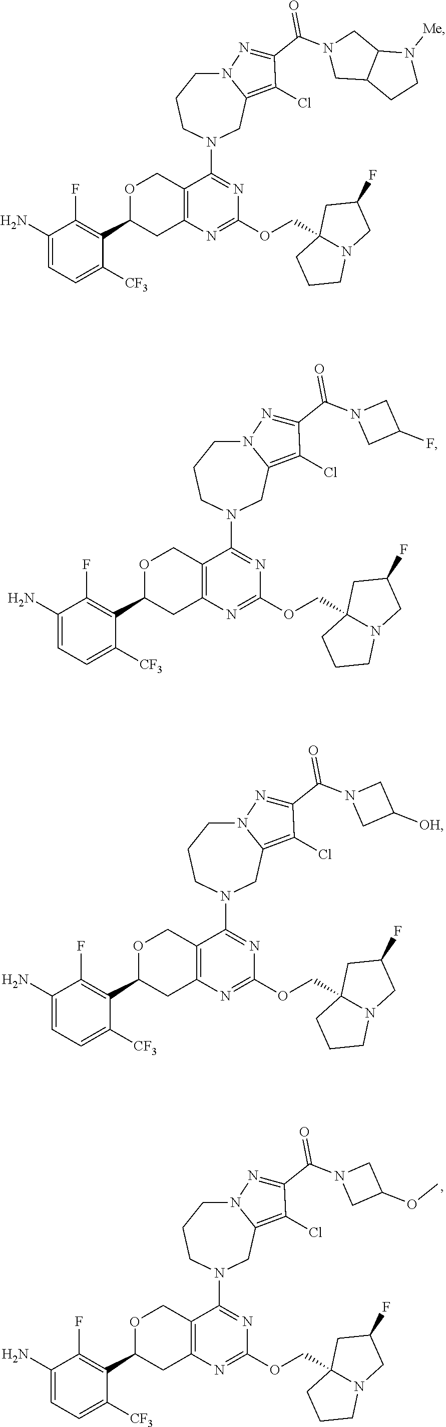 [SMILES: CN1CCC2CN(C(=O)c3nn4c(c3Cl)CN(c3nc(OC[C@@]56CCCN5C[C@H](F)C6)nc5c3CO[C@H](c3c(C(F)(F)F)ccc(N)c3F)C5)CCC4)CC21 ; Nc1ccc(C(F)(F)F)c([C@@H]2Cc3nc(OC[C@@]45CCCN4C[C@H](F)C5)nc(N4CCCn5nc(C(=O)N6CC(F)C6)c(Cl)c5C4)c3CO2)c1F ; Nc1ccc(C(F)(F)F)c([C@@H]2Cc3nc(OC[C@@]45CCCN4C[C@H](F)C5)nc(N4CCCn5nc(C(=O)N6CC(O)C6)c(Cl)c5C4)c3CO2)c1F ; COC1CN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1]

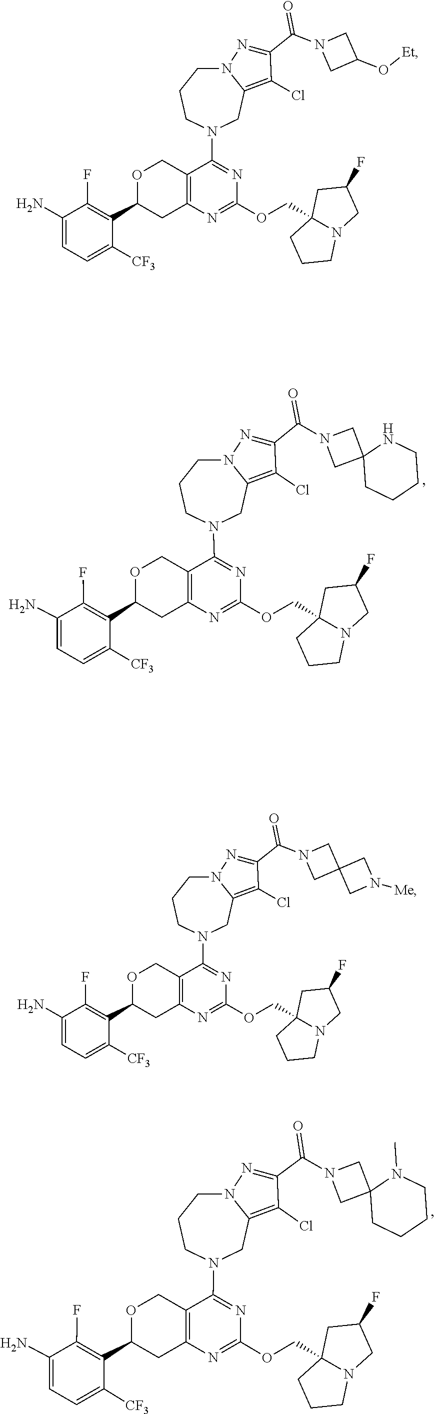 [SMILES: CCOC1CN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1 ; Nc1ccc(C(F)(F)F)c([C@@H]2Cc3nc(OC[C@@]45CCCN4C[C@H](F)C5)nc(N4CCCn5nc(C(=O)N6CC7(CCCCN7)C6)c(Cl)c5C4)c3CO2)c1F ; CN1CC2(C1)CN(C(=O)c1nn3c(c1Cl)CN(c1nc(OC[C@@]45CCCN4C[C@H](F)C5)nc4c1CO[C@H](c1c(C(F)(F)F)ccc(N)c1F)C4)CCC3)C2 ; CN1CCCCC12CN(C(=O)c1nn3c(c1Cl)CN(c1nc(OC[C@@]45CCCN4C[C@H](F)C5)nc4c1CO[C@H](c1c(C(F)(F)F)ccc(N)c1F)C4)CCC3)C2]

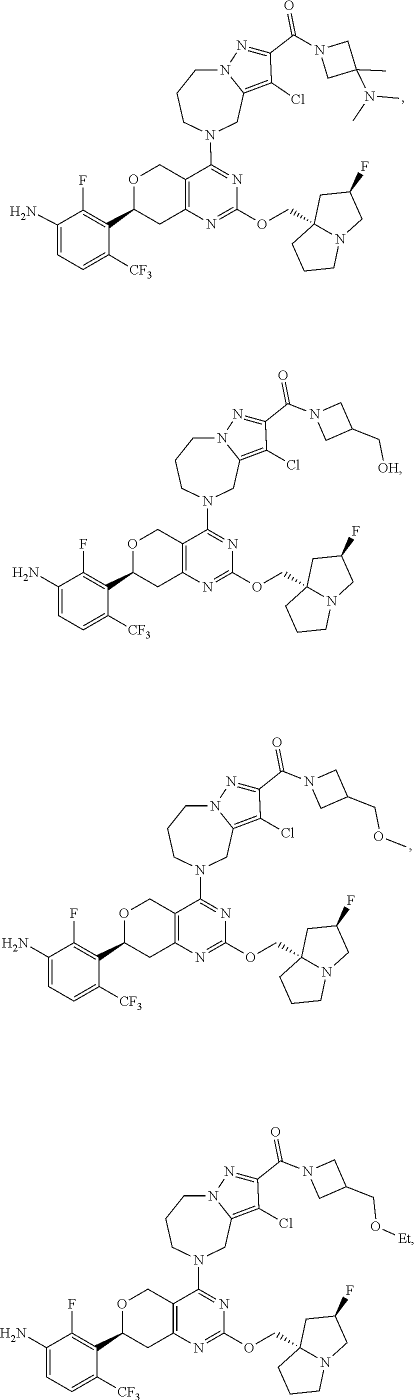 [SMILES: CN(C)C1(C)CN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1 ; Nc1ccc(C(F)(F)F)c([C@@H]2Cc3nc(OC[C@@]45CCCN4C[C@H](F)C5)nc(N4CCCn5nc(C(=O)N6CC(CO)C6)c(Cl)c5C4)c3CO2)c1F ; COCC1CN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1 ; CCOCC1CN(C(=O)c2nn3c(c2Cl)CN(c2nc(OC[C@@]45CCCN4C[C@H](F)C5)nc4c2CO[C@H](c2c(C(F)(F)F)ccc(N)c2F)C4)CCC3)C1]

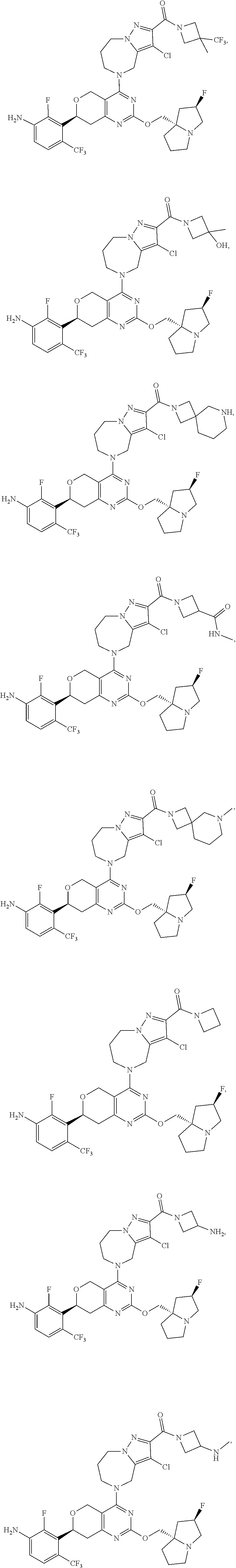

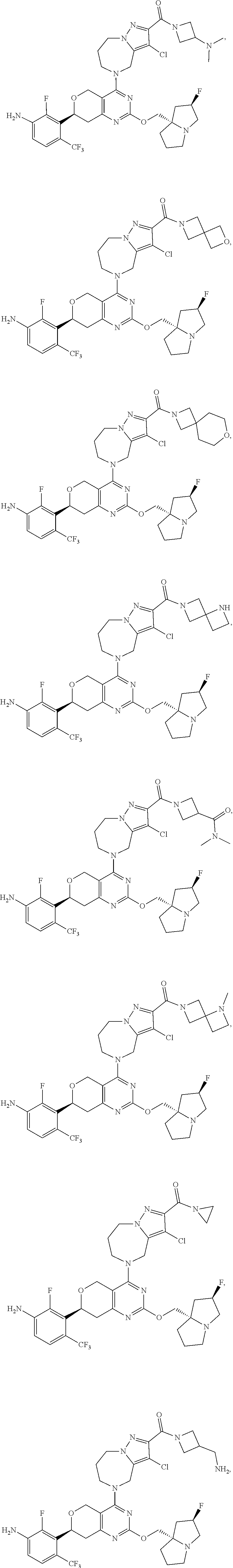

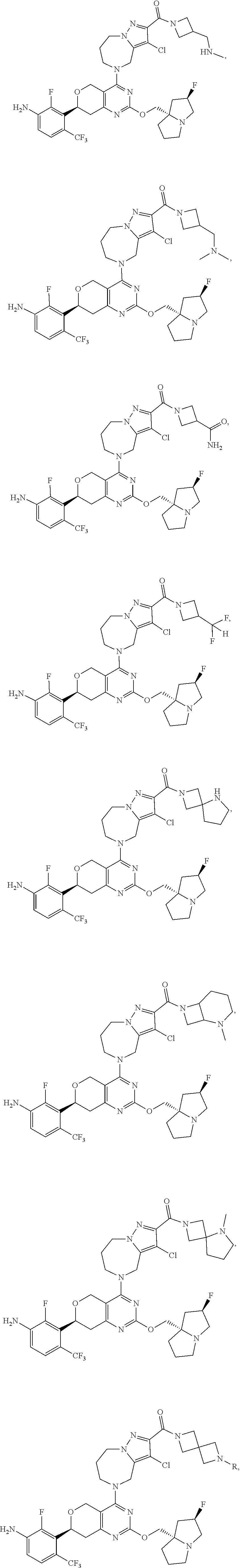

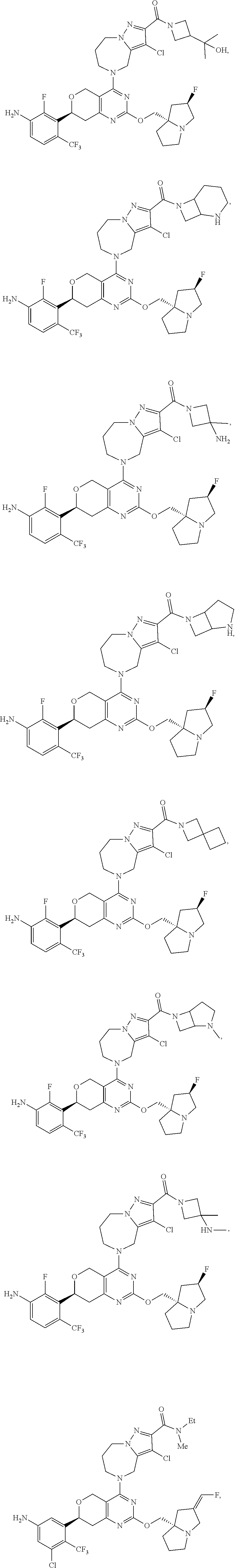

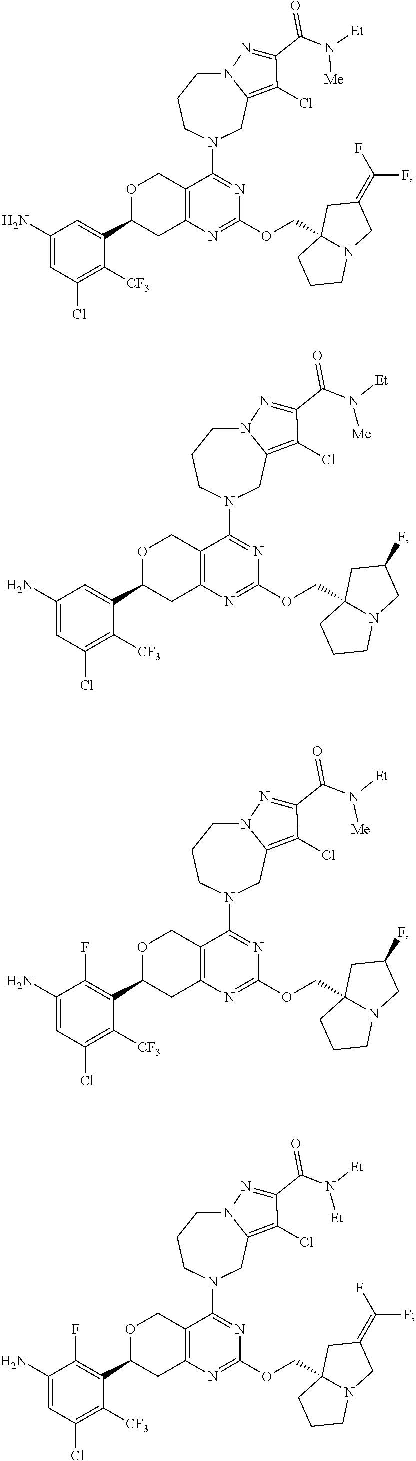 [SMILES: CCN(C)C(=O)c1nn2c(c1Cl)CN(c1nc(OC[C@@]34CCCN3CC(=C(F)F)C4)nc3c1CO[C@H](c1cc(N)cc(Cl)c1C(F)(F)F)C3)CCC2 ; CCN(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2 ; CCN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2 ; CCN(CC)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2]

or a stereoisomer, a tautomer, or a pharmaceutically acceptable salt thereof.

In some embodiments, the compound is selected from the compounds of Table 1a and 1b, or a stereoisomer, a tautomer, or a pharmaceutically acceptable salt thereof.

In some embodiments, the compound is selected from the compounds of Table 1a, or a stereoisomer, a tautomer, or a pharmaceutically acceptable salt thereof. In some embodiments, the compound is selected from the compounds of Table 1b, or a stereoisomer, a tautomer, or a pharmaceutically acceptable salt thereof.

In each of the preceding embodiments of specific compound above, the deuterated forms below may be employed:

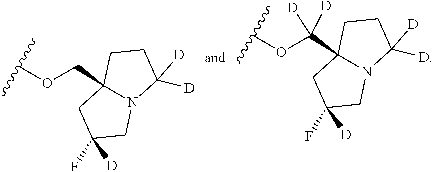

The compounds disclosed herein can exist as salts. The present embodiments include such salts, which can be pharmaceutically acceptable salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures) thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds disclosed herein contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts includes salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds disclosed herein can exist in unsolvated forms as well as solvated forms, including hydrated forms. In general, the solvated forms are equivalent to unsolvated forms and are encompassed within the scope of the present embodiments. Certain compounds disclosed herein may exist in multiple crystalline or amorphous forms. In general, all physical forms are equivalent for the uses contemplated by the present embodiments and are intended to be within the scope of the present embodiments.

Certain compounds disclosed herein possess asymmetric carbon atoms (optical centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present embodiments. The compounds disclosed herein do not include those which are known in art to be too unstable to synthesize and/or isolate. The present embodiments are meant to include compounds in racemic and optically pure forms. Optically active (R)- and (S)-, or (D)- and (L)-isomers may be prepared using chiral synthons or chiral reagents, or resolved using conventional techniques. The compounds disclosed herein can be provided as a mixture of atropisomers or can be pure atropisomers.

Isomers include compounds having the same number and kind of atoms, and hence the same molecular weight, but differing in respect to the structural arrangement or configuration of the atoms.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the embodiments.

Unless otherwise stated, the compounds disclosed herein may also contain unnatural proportions of atomic isotopes at one or more of the atoms that constitute such compounds. For example, the compounds disclosed herein may be labeled with radioactive or stable isotopes, such as for example deuterium (²H), tritium (3H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds disclosed herein, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds disclosed herein. Additionally, prodrugs can be converted to the compounds disclosed herein by chemical or biochemical methods in an ex vivo environment. For example, prodrugs can be slowly converted to the compounds disclosed herein when placed in a transdermal patch reservoir with a suitable enzyme or chemical reagent.

Compounds disclosed herein can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. The following synthetic reaction schemes are merely illustrative of some methods by which the compounds disclosed herein can be synthesized, and various modifications to these synthetic reaction schemes can be made and will be suggested to one skilled in the art having referred to the disclosure contained herein.

For illustrative purposes, reaction Schemes below provide routes for synthesizing the compounds disclosed herein as well as key intermediates. For a more detailed description of the individual reaction steps, see the Examples section below. Those skilled in the art will appreciate that other synthetic routes may be used. Although some specific starting materials and reagents are depicted in the Schemes and discussed below, other starting materials and reagents can be substituted to provide a variety of derivatives or reaction conditions. In addition, many of the compounds prepared by the methods described below can be further modified in light of this disclosure using conventional chemistry well known to those skilled in the art.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, or, about 20° C.

Some compounds in following schemes are depicted with generalized substituents; however, one skilled in the art will immediately appreciate that the nature of the substituents can varied to afford the various compounds contemplated in the present embodiments. Moreover, the reaction conditions are exemplary and alternative conditions are well known. The reaction sequences in the following examples are not meant to limit the scope of the embodiments as set forth in the claims.

### IV. Pharmaceutical Formulations

In some embodiments, pharmaceutical compositions comprise a compound of any one of the compounds disclosed herein and a pharmaceutically acceptable excipient.

In some embodiments, there is provided a pharmaceutical composition comprising a pharmaceutically effective amount of a compound of any one of Formula (I), or a pharmaceutically acceptable salt thereof, and a pharmaceutically acceptable excipient.

In some embodiments, the pharmaceutical composition further comprises an additional therapeutic agent.

In some embodiments, the additional therapeutic agent is a chemotherapeutic agent. In some embodiments, the chemotherapeutic agent is an anti-microtubule agent, a platinum coordination complex, an alkylating agent, an antibiotic agent, a topoisomerase II inhibitor, an antimetabolite, a topoisomerase I inhibitor, a hormone or hormonal analogue, a signal transduction pathway inhibitor, a non-receptor tyrosine kinase angiogenesis inhibitor, an immunotherapeutic agent, a proapoptotic agent, an inhibitor of LDH-A, an inhibitor of fatty acid biosynthesis, a cell cycle signalling inhibitor, a HDAC inhibitor, a proteasome inhibitor, or an inhibitor of cancer metabolism. In some embodiments, the chemotherapeutic agent is cisplatin, carboplatin, doxorubicin, ionizing radiation, docetaxel or paclitaxel.

The compounds disclosed herein can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds disclosed herein can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds disclosed herein can be administered transdermally. The compounds disclosed herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present embodiments also provide pharmaceutical compositions including one or more pharmaceutically acceptable carriers and/or excipients and either a compound of Formula I, or a pharmaceutically acceptable salt of a compound of Formula I.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other exceipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations can also be used orally using, for example, push-fit capsules made of gelatin, as well as soft, sealed capsules made of gelatin and a coating such as glycerol or sorbitol. Push-fit capsules can contain the compounds disclosed herein mixed with a filler or binders such as lactose or starches, lubricants such as talc or magnesium stearate, and, optionally, stabilizers. In soft capsules, the compounds disclosed herein may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds disclosed herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds disclosed herein can be delivered by transdermally, by a topical route, formulated as applicator sticks, solutions, suspensions, emulsions, gels, creams, ointments, pastes, jellies, paints, powders, and aerosols.

The compounds disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical formulations of the compounds disclosed herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

The pharmaceutical formulations of the compounds disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

In some embodiments, the formulations of the compounds disclosed herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR and/or MR modulator and disease or condition treated.

Single or multiple administrations of the compounds disclosed herein formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compounds disclosed herein is in a daily amount of between about 0.5 to about 30 mg per kilogram of body weight per day. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compounds disclosed herein for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987).

The compounds described herein can be used in combination with one another, with other active agents known to be useful in modulating a glucocorticoid receptor, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another.

After a pharmaceutical composition including a compound disclosed herein has been formulated in one or more acceptable carriers, it can be placed in an appropriate container and labeled for treatment of an indicated condition. For administration of the compounds of Formula I, such labeling would include, e.g., instructions concerning the amount, frequency and method of administration.

In some embodiments, the compositions disclosed herein are useful for parenteral administration, such as intravenous (IV) administration or administration into a body cavity or lumen of an organ. The formulations for administration will commonly comprise a solution of the compositions disclosed herein dissolved in one or more pharmaceutically acceptable carriers. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, tonicity adjusting agents, e.g., sodium acetate, sodium chloride, potassium chloride, calcium chloride, sodium lactate and the like. The concentration of the compositions in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions disclosed herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions disclosed herein into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### V. Methods

In some embodiments, there is provided a method of treating a disorder or condition in a subject, the method comprising administering to the human a therapeutically effective amount of a compound disclosed herein, or a pharmaceutically acceptable salt thereof, or a pharmaceutical composition as disclosed herein.

In some embodiments, there is provided a method for inhibiting KRAS G12D or G12V activity in a cell, comprising contacting the cell in which inhibition of KRAS G12D or G12V activity is desired with an effective amount of a compound disclosed herein or a pharmaceutically acceptable salt thereof.

In some embodiments, there is provided a method for inhibiting KRAS G12D or G12V activity in a cell, comprising contacting the cell in which inhibition of KRAS G12D or G12V activity is desired with the pharmaceutical composition disclosed herein.

In some embodiments, there is provided a method for treating a KRAS G12D or G12V-associated cancer comprising administering to a patient in need thereof a therapeutically effective amount of a compound disclosed herein, or a pharmaceutically acceptable salt thereof.

In some embodiments, there is provided a method for treating a KRAS G12D or G12V-associated cancer comprising administering to a patient in need thereof the pharmaceutical composition disclosed herein.

In some embodiments, there is provided a method of treating a subject having cancer, the cancer characterized by the presence of a KRAS G12D or G12V mutation, the method comprising administering to the human a therapeutically effective amount of a compound of any one of Formula (I), or a pharmaceutically acceptable salt thereof, or a a pharmaceutical composition as disclosed herein.

In some embodiments, there is provided a method for manufacturing a medicament for treating a subject having cancer, the cancer characterized by the presence of a KRAS G12D or G12V mutation, the compound comprising Formula (I), or a pharmaceutically acceptable salt thereof, or a pharmaceutical composition.

In some embodiments, there is provided a use of a compound of Formula (I), or a pharmaceutically acceptable salt thereof, or a a pharmaceutical composition as disclosed herein, for the manufacture of a medicament for the treatment in a human having cancer, the cancer characterized by the presence of a KRAS G12D or G12V mutation.

In some embodiments, there are provided compounds of Formula (I), or a pharmaceutically acceptable salt thereof, or a a pharmaceutical composition as disclosed herein, for use in the treatment of a subject having cancer, the cancer characterized by the presence of a KRAS G12D or G12V mutation.

In some embodiments, there is provided a method for treating cancer in a patient in need thereof, the method comprising (a) determining that the cancer is associated with a KRAS G12D or G12V mutation (e.g., a KRAS G12D or G12V-associated cancer); and (b) administering to the patient a therapeutically effective amount of a compound disclosed herein.

In some embodiments, there is provided a method for treating cancer in a patient in need thereof, the method comprising (a) determining that the cancer is associated with a KRAS G12D or G12V mutation (e.g., a KRAS G12D or G12V-associated cancer); and (b) administering to the patient the pharmaceutical composition disclosed herein.

In some embodiments, the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

In some embodiments, the cancer is non-small cell lung cancer, small cell lung cancer, colorectal cancer, rectal cancer, or pancreatic cancer.

In certain embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds of Formula (I) or a pharmaceutically acceptable salt thereof, can be inhibitors of KRAS G12D or G12V. For example, the inhibition constant (Ki) of the compounds disclosed herein can be less than about 50 μM, or less than about 40, 30, 20, 10, 9, 8, 7, 6, 5, 4, 3, 2, or less than about 1 μM. The inhibition constant (Ki) of the compounds disclosed herein can be less than about 1,000 nM, or less than about 900, 800, 700, 600, 500, 400, 300, 200, 100, 90, 80, 70, 60, 50, 40, 30, 20, 10, 9, 8, 7, 6, 5, 4, 3, 2, or less than about 1 nM. The inhibition constant (Ki) of the compounds disclosed herein can be less than about 1 nM, or less than about 0.9, 0.8, 0.7, 0.6, 0.5, 0.4, 0.3, 0.2, or less than about 0.1 nM.

The compounds of Formula (I) or a pharmaceutically acceptable salt thereof, can be selective inhibitors of KRAS G12D or G12V. For example, KRAS G12D or G12V inhibition constant (IC50) of the compounds disclosed herein can be at least 2-fold less than the inhibition constant of one or more of KRAS wild-type, or NRAS, or HRAS, or at least 3, 4, 5, 6, 7, 8, 9, 10, 20, 30, 40, 50, 60, 70, 80, 90 or 100-fold less. The KRAS G12D or G12V inhibition constant (Ki) of the compounds disclosed herein can also be at least 100-fold less than the inhibition constant of one or more of KRAS wild-type, or NRAS, or HRAS, or at least 200, 300, 400, 500, 600, 700, 800, 900, 1000, or 10,000-fold less.

### A. Cancer Combination Therapies

The compounds disclosed herein or salts thereof may be employed alone or in combination with other agents for treatment. For example, the second agent of the pharmaceutical combination formulation or dosing regimen may have complementary activities to the compounds disclosed herein such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound disclosed herein or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound disclosed herein, in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with embodiments herein. For example, a compound disclosed herein may be administered with another therapeutic agent simultaneously or sequentially in separate unit dosage forms or together in a single unit dosage form. Accordingly, the present embodiments provide a single unit dosage form comprising a compound of Formula (I), an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In certain embodiments, compositions disclosed herein are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound disclosed herein or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵⁵, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®., Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C₄, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds disclosed herein include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGi k antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C₂₂₅ or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2-methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-111 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA@); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (RI1577); orafenib, ABT510; Bcl-2 inhibitor such as oblimersen sodium (GENASENSE®); pixantrone; farnesyltransferase inhibitors such as lonafarnib (SCH 6636, SARASAR™; and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Chemotherapeutic agents also include treatments for Alzheimer's Disease such as donepezil hydrochloride and rivastigmine; treatments for Parkinson's Disease such as L-DOPA/carbidopa, entacapone, ropinrole, pramipexole, bromocriptine, pergolide, trihexephendyl, and amantadine; agents for treating multiple sclerosis (MS) such as beta interferon (e.g., Avonex® and Rebif®), glatiramer acetate, and mitoxantrone; treatments for asthma such as albuterol and montelukast sodium; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Additionally, chemotherapeutic agents include pharmaceutically acceptable salts, acids or derivatives of any of chemotherapeutic agents, described herein, as well as combinations of two or more of them.

### VI. Examples

### Abbreviations

ACN—acetonitrile
AC₂O—acetyl acetate
BINAP—(+/−)-2,2′-bis(diphenylphosphino)-1,1′-binaphthyl
Boc₂O—di-tert-butyl dicarbonate
BOP—(benzotriazol-1-yloxy)tris(dimethylamino)phosphonium hexafluorophosphate
DBU—1,8-diazabicyclo[5.4.0]undec-7-ene
DCE—1,2-dichloroethane
DCM—dichloromethane
DIEA or DIPEA—N,N-diisopropylethylamine
DMA—N,N-dimethylacetamide
DMAc—N,N-dimethylacetamide
DMAP—4-dimethylaminopyridine
DMF—N,N-dimethylformamide
DMSO—dimethyl sulfoxide
EA—ethyl acetate
EtOAc—ethyl acetate
EtOH—ethanol
HATU—2-(7-azabenzotriazol-1-yl)-N,N,N′,N′-tetramethyluronium hexafluorophosphate
HFIP—hexafluoroisopropanol
HOAc—acetic acid
iPrOAc—isopropyl acetate
KF—potassium fluoride
KOAc—potassium acetate
LDA—lithium diisopropylamide
LiHMDS—lithium bis(trimethylsilyl)amide
mCPBA—3-chloroperoxybenzoic acid
MeCN—acetonitrile
Mel—iodomethane
MeOH—methanol
MeONa—sodium methoxide or sodium methanolate
MTBE—methyl tert-butyl ether
MW—microwave
NaBH(OAc)₃—sodium triacetoxyborohydride
NIS—N-iodosuccinimide
P(Cy)₃ or PCy₃—tricyclohexylphosphine
P(t-Bu)₃HBF₄— tri-tert-butyl phosphonium tetrafluoroborate
Pd/C—palladium on carbon
Pd₂(dba)₃—tris(dibenzylideneacetone)dipalladium(0)
Pd₂(dba)₃CHCl₃—tris(dibenzylidenacetone)dipalladium(O), complexed with chloroform
Pd(dppf)Cl₂·CH₂Cl₂—[1,1′-bis(diphenylphosphino)ferrocene]dichloropalladium(II) or dichloro[1.1′-bis(diphenylphosphino)ferrocene]palladium(II), complexed with dichloromethane
Pd(PPh₃)₄—tetrakis(triphenylphosphine)palladium(O)
Pd(PPh₃)₂Cl₂—bis(triphenylphosphine)palladium(II) dichloride
PE—petroleum ether
PMBCl—4-methoxybenzylchloride
pTsA—p-toluenesulfonic acid
r.t.—room temperature
Sn₂(n-Bu)₆—hexabutylditin
TBSCl—tert-butyldimethylsilyl chloride or tert-butyldimethylchlorosilane
[Rh(COD)Cl]₂— chloro(1,5-cyclooctadiene)rhodium(I) dimer
TEA—triethylamine
TFA—trifluoroacetic acid or 2,2,2-trifluoroacetic acid
THF—tetrahydrofuran
THP—tetrahydropyran
TsOH—p-toluenesulfonic acid

### A. Synthetic Procedures

### General Procedure

The compounds disclosed may be prepared from commercially available reagents using the synthetic methods and reaction schemes herein, or using other reagents and conventional methods well known to those skilled in the art. For instance, compounds of the present invention may be prepared according to the general reaction Scheme Ia and Ib:

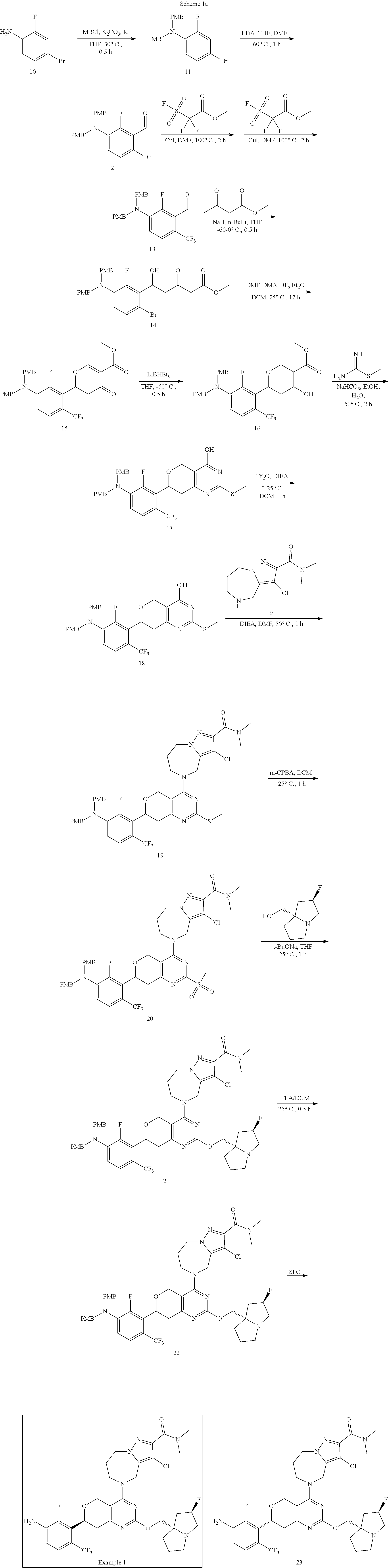

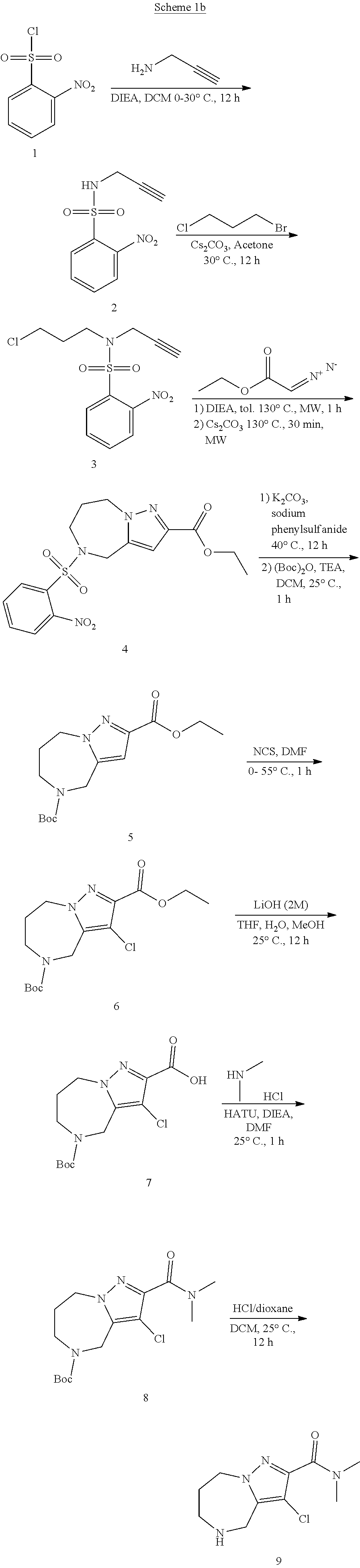

### 5-((S)-7-(3-Amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chlo ro-N,N-dimethyl-5,6,7,8-tetrahydro-4H-pyrazolo[1,5-a][1,4]diazepine-2-carboxamide

### Example 1

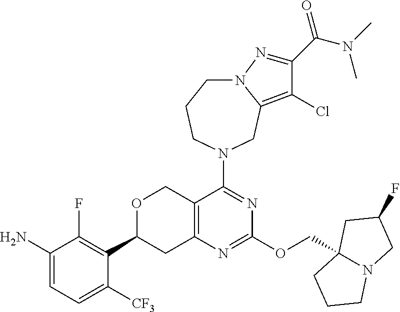 [SMILES: CN(C)C(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2]

### 5-((R)-7-(3-Amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chlo ro-N,N-dimethyl-5,6,7,8-tetrahydro-4H-pyrazolo[1,5-a][1,4]diazepine-2-carboxamide (23)

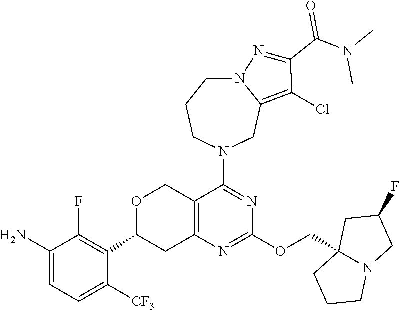 [SMILES: CN(C)C(=O)c1nn2c(c1Cl)CN(c1nc(OC[C@@]34CCCN3C[C@H](F)C4)nc3c1CO[C@@H](c1c(C(F)(F)F)ccc(N)c1F)C3)CCC2]

### 2-Nitro-N-(prop-2-yn-1-yl)benzenesulfonamide (2)

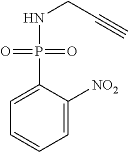

To a solution of prop-2-yn-1-amine (1) (1.72 g, 31.23 mmol, 2 mL) in DCM (80 mL) was added DIEA (7.42 g, 57.41 mmol, 10 mL) and 2-nitrobenzenesulfonyl chloride (6.46 g, 29.15 mmol) at 0° C. The mixture was stirred at 30° C. for 12 hours. LCMS showed clean conversion. The mixture was concentrated under reduced pressure. The residue was purified by flash silica gel chromatography (40 g SEPAFLASH® Silica Flash Column, Eluent of 30~50% Ethyl acetate/Petroleum ether gradient 30 mL/min) to afford the title compound (2) (5.2 g, 21.65 mmol, 74.26% yield) as a yellow solid. LCMS (ESI, m/z): 241.1 [M+H]+. ¹H NMR (400 MHz, CDCl₃) δ 8.24-8.17 (m, 1H), 7.96-7.89 (m, 1H), 7.80-7.74 (m, 2H), 5.71 (t, J=8.0 Hz, 1H), 4.03 (dd, J=2.4, 6.4 Hz, 2H), 1.98 (t, J=2.4 Hz, 1H).

### N-(3-chloropropyl)-2-nitro-N-(prop-2-yn-1-yl)benzenesulfonamide (3)

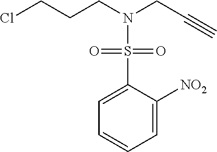 [SMILES: C#CCN(CCCCl)S(=O)(=O)c1ccccc1[N+](=O)[O-]]

To a solution of 2-nitro-N-prop-2-ynyl-benzenesulfonamide (2) (5.2 g, 21.65 mmol) in MeCN (80 mL) was added Cs₂CO₃ (21.16 g, 64.94 mmol) and 1-bromo-3-chloro-propane (17.04 g, 108.23 mmol, 10.68 mL). The mixture was stirred at 30° C. for 12 hours. LCMS showed clean conversion. The mixture was filtered off, the filter-cake was washed by EtOAc (20 mL×2), and filtrate was concentrated under reduced pressure. The residue was purified by flash silica gel chromatography (40 g SEPAFLASH® Silica Flash Column, Eluent of 30~50% Ethyl acetate/Petroleum ether gradient 30 mL/min) to afford the title compound (3) (6 g, 18.94 mmol, 87.51% yield) as a yellow oil. LCMS (ESI, m/z): 317.1 [M+H]⁺. ¹H NMR (400 MHz, CDCl₃) δ 8.10-8.06 (m, 1H), 7.74-7.64 (m, 3H), 4.23 (d, J=2.4 Hz, 2H), 3.61-3.55 (m, 4H), 2.22 (t, J=2.4 Hz, 1H), 2.14-2.07 (m, 2H).

### Ethyl 5-((2-nitrophenyl)sulfonyl)-5,6,7,8-tetrahydro-4H-pyrazolo[1,5-a][1,4]diazepine-2-carboxylate (4)

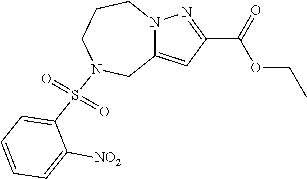 [SMILES: CCOC(=O)c1cc2n(n1)CCCN(S(=O)(=O)c1ccccc1[N+](=O)[O-])C2]

N-(3-chloropropyl)-2-nitro-N-prop-2-ynyl-benzenesulfonamide (600 mg, 1.89 mmol) (3), ethyl 2-diazoacetate (324.20 mg, 2.84 mmol, 298.80 μL) and DIEA (244.81 mg, 1.89 mmol, 329.93 μL) were taken up into a microwave tube in toluene (10 mL). The sealed tube was heated at 130° C. for 1 hour under microwave. Temperature was cooled to 25° C., Cs₂CO₃ (678.87 mg, 2.08 mmol) was added to the tube, the sealed tube was heated at 130° C. for 30 min. LCMS showed complete conversion. The reaction mixture was added H₂O (60 mL), and extracted with EtOAc (50 mL×3). The combined organic layers were washed with brine (10 mL), dried over Na₂SO₄, filtered and concentrated under reduced pressure. The residue was purified by flash silica gel chromatography (20 g SEPAFLASH® Silica Flash Column, Eluent of 50~90% Ethyl acetate/Petroleum ether gradient 30 mL/min) to afford the title compound (4) (950 mg, 1.73 mmol, 22.89% yield, 72% purity) as a yellow solid. LCMS (ESI, m/z): 395.1 [M+H]⁺. ¹H NMR (400 MHz, CDCl₃) δ 7.98 (dd, J=0.8, 7.2 Hz, 1H), 7.71-7.62 (m, 3H), 6.77 (s, 1H), 4.59 (s, 2H), 4.54-4.47 (m, 2H), 4.41-4.36 (m, 2H), 3.74-3.67 (m, 2H), 2.11-2.03 (m, 2H), 1.39 (t, J=7.2 Hz, 3H).

### 5-(Tert-butyl) 2-ethyl 7,8-dihydro-4H-pyrazolo[1,5-a][1,4]diazepine-2,5(6H)-dicarboxylate (5)

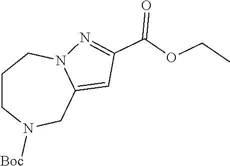 [SMILES: CCOC(=O)c1cc2n(n1)CCCN(C(=O)OC(C)(C)C)C2]

To a solution of ethyl 5-(2-nitrophenyl)sulfonyl-4,6,7,8-tetrahydropyrazolo[1,5-a][1,4]diazepine-2-carboxylate (4) (1 g, 2.54 mmol) in MeCN (2 mL) was added sodium phenylsulfanide (1.01 g, 7.61 mmol) and K₂CO₃ (1.05 g, 7.61 mmol). The mixture was stirred at 40° C. for 12 hours. The mixture was filtered off, filter-cake was washed by EtOAc (20 mL×3), the filtrate was concentrated under reduced pressure to afford crude ethyl 5,6,7,8-tetrahydro-4H-pyrazolo[1,5-a][1,4]diazepine-2-carboxylate (530 mg, crude) as a yellow oil. To a solution of ethyl 5,6,7,8-tetrahydro-4H-pyrazolo[1,5-a][1,4]diazepine-2-carboxylate (530 mg, 2.53 mmol) in THF (6 mL) was added TEA (768.91 mg, 7.60 mmol, 1.06 mL) and (Boc)₂O (608.08 mg, 2.79 mmol, 640.09 L). The mixture was stirred at 25° C. for 1 hour. LCMS showed complete conversion. The reaction mixture was added H₂O (20 mL), and extracted with EtOAc (20 mL×3). The combined organic layers were washed with brine (10 mL), dried over Na₂SO₄, filtered and concentrated under reduced pressure. The residue was purified by flash silica gel chromatography (20 g SEPAFLASH® Silica Flash Column, Eluent of 25~40% Ethyl acetate/Petroleum ether gradient 25 mL/min) to afford the title compound (5) (770 mg, 2.49 mmol, 98.27% yield) as a yellow solid. LCMS (ESI, m/z): 310.2 [M+H]⁺. ¹H NMR (400 MHz, CDCl₃) δ 6.82-6.60 (m, 1H), 4.52-4.33 (m, 6H), 3.72 (s, 2H), 1.94 (s, 2H), 1.41 (s, 9H), 1.40-1.36 (m, 3H).

### 5-(Tert-butyl) 2-ethyl 3-chloro-7,8-dihydro-4H-pyrazolo[1,5-a][1,4]diazepine-2,5(6H)-dicarboxylate (6)

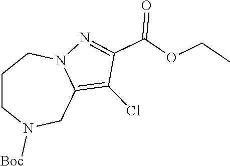 [SMILES: CCOC(=O)c1nn2c(c1Cl)CN(C(=O)OC(C)(C)C)CCC2]

To a solution of 5-tert-butyl 2-ethyl 4,6,7,8-tetrahydropyrazolo[1,5-a][1,4]diazepine-2,5-dicarboxylate (5) (700 mg, 2.26 mmol) in DMF (10 mL) was added NCS (362.58 mg, 2.72 mmol) at 0° C. The mixture was stirred at 55° C. for 1 hour. LCMS showed complete conversion. The reaction mixture was added to H₂O (10 mL), and extracted with EtOAc (10 mL×3). The combined organic layers were washed with brine (10 mL), dried over Na₂SO₄, filtered and concentrated under reduced pressure. The residue was purified by flash silica gel chromatography (12 g SEPAFLASH® Silica Flash Column, Eluent of 30~50% Ethyl acetate/Petroleum ether gradient 15 mL/min) to afford the title compound (750 mg, 2.18 mmol, 96.41% yield) as a white solid. LCMS (ESI, m/z): 344.2 [M+H]⁺. ¹H NMR (400 MHz, CDCl₃) δ 4.56-4.47 (m, 4H), 4.46-4.38 (m, 2H), 3.75 (s, 2H), 1.98 (s, 2H), 1.44 (s, 9H), 1.43-1.39 (m, 3H).

### 5-(Tert-butoxycarbonyl)-3-chloro-5,6,7,8-tetrahydro-4H-pyrazolo[1,5-a][1,4]diazepine-2-carboxylic acid (7)

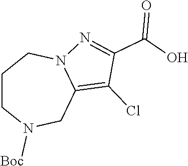 [SMILES: CC(C)(C)OC(=O)N1CCCn2nc(C(=O)O)c(Cl)c2C1]

To a solution of 5-tert-butyl 2-ethyl 3-chloro-4,6,7,8-tetrahydropyrazolo[1,5-a][1,4]diazepine-2,5-dicarboxylate (6) (750 mg, 2.18 mmol) in THF (3 mL) was added LiGH (2 M, 2.18 mL), MeOH (2 mL) and H₂O (1 mL). The mixture was stirred at 25° C. for 12 hours. LCMS showed complete conversion. THF was removed under reduced pressure, pH of the residue was adjusted to 4-5 by adding HCl (0.5 M), and extracted with EtOAc (30 mL×3). The combined organic layers were washed with brine (15 mL), dried over Na₂SO₄, filtered and concentrated under reduced pressure to afford the title compound (7) (650 mg, 2.06 mmol, 94.37% yield) as a white solid. LCMS (ESI, m/z): 316.2 [M+H]⁺. ¹H NMR (400 MHz, CDCl₃) δ 4.58-4.48 (m, 4H), 3.77 (s, 2H), 1.99 (s, 2H), 1.44 (s, 9H).

### Tert-butyl 3-chloro-2-(dimethylcarbamoyl)-7,8-dihydro-4H-pyrazolo[1,5-a][1,4]diazepine-5(6H)-carboxylate (8)

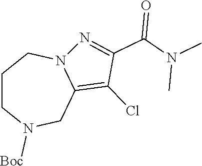 [SMILES: CN(C)C(=O)c1nn2c(c1Cl)CN(C(=O)OC(C)(C)C)CCC2]

To a solution of 5-tert-butoxycarbonyl-3-chloro-4,6,7,8-tetrahydropyrazolo[1,5-a][1,4]diazepine-2-carboxylic acid (7) (650 mg, 2.06 mmol) in DMF (2 mL) was added HATU (1.17 g, 3.09 mmol), dimethylamine hydrochloride (185.62 mg, 2.28 mmol, 208.56 L) and DIEA (1.06 g, 8.23 mmol, 1.43 mL). The mixture was stirred at 25° C. for 1 hour. LCMS showed complete conversion. The reaction mixture was added H₂O (10 mL), and extracted with EtOAc (20 mL×3). The combined organic layers were washed with brine (10 mL), dried over Na₂SO₄, filtered and concentrated under reduced pressure. The residue was purified by flash silica gel chromatography (12 g SEPAFLASH® Silica Flash Column, Eluent of 50~90% Ethyl acetate/Petroleum ether gradient 15 mL/min) to afford the title compound (8) (700 mg, 2.04 mmol, 99.19% yield) as a yellow oil. LCMS (ESI, m/z): 343.2 [M+H]⁺. ¹H NMR (400 MHz, CDCl₃) δ 4.50 (s, 2H), 4.44-4.37 (m, 2H), 3.75 (s, 2H), 3.10 (d, J=4.0 Hz, 6H), 1.97 (s, 2H), 1.44 (s, 9H).

### 3-Chloro-N,N-dimethyl-5,6,7,8-tetrahydro-4H-pyrazolo[1,5-a][1,4]diazepine-2-carboxamide (9)

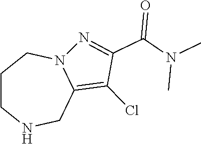 [SMILES: CN(C)C(=O)c1nn2c(c1Cl)CNCCC2]

To a solution of tert-butyl 3-chloro-2-(dimethylcarbamoyl)-4,6,7,8-tetrahydropyrazolo[1,5-a][1,4]diazepine-5-carboxylate (8) (500 mg, 1.46 mmol) in DCM (2 mL) was added HCl/dioxane (4 M, 1 mL). The mixture was stirred at 25° C. for 12 hours. LCMS showed clean conversion. The mixture was concentrated under reduced pressure to afford the title compound (9) (400 mg, crude, HCl) as a white solid. LCMS (ESI, m/z): 243.1 [M+H]⁺.

### 4-Bromo-2-fluoro-N,N-bis[(4-methoxyphenyl)methyl]aniline (11)

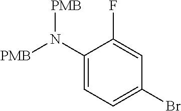

To a solution of 4-bromo-2-fluoro-aniline (10) (10.0 g, 52.63 mmol) in THF (50 mL) was added PMB-Cl (18.13 g, 115.78 mmol, 15.71 mL), KI (8.74 g, 52.63 mmol) and K₂CO₃ (18.18 g, 131.57 mmol). The mixture was stirred at 30° C. for 0.5 hour. TLC showed complete conversion. The reaction mixture was quenched by the addition of saturated aqueous NH₄Cl (500 ml) and extracted with EtOAc (500 mL×2). The combined organic layers were dried over Na₂SO₄, filtered and concentrated under reduced pressure. The residue was purified by column chromatography (SiO₂, Petroleum ether/Ethyl acetate=100/1 to 10/1) to afford the title compound (11) (22.3 g, 51.82 mmol, 98.47% yield) as a yellow oil. ¹H NMR (400 MHz, CDCl₃) δ 7.13-7.19 (m, 4H), 7.03 (dd, J=8.8, 1.2 Hz, 1H), 6.87-6.93 (m, 1H), 6.83 (d, J=8.8 Hz, 4H), 6.61-6.72 (m, 1H), 4.20 (br s, 4H), 3.79 (s, 6H).

### 3-[bis[(4-Methoxyphenyl)methyl]amino]-6-bromo-2-fluoro-benzaldehyde (12)

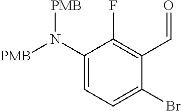

To a solution of 4-bromo-2-fluoro-N,N-bis[(4-methoxyphenyl)methyl]aniline (22 g, 51.13 mmol) (11) in THF (300 mL) was added dropwise LDA (2 M, 102.25 mL) at −60° C., the mixture was stirred at −60° C. for 0.5 hour, then DMF (88.00 g, 1.20 mol, 92.63 mL) was added. The mixture was stirred at −60° C. for 0.5 hour. TLC showed complete conversion. The reaction mixture was quenched by the addition of saturated aqueous NH₄Cl (500 ml) and extracted with EtOAc (500 mL×2). The combined organic layers were dried over Na₂SO₄, filtered and concentrated under reduced pressure. The residue was purified by column chromatography (SiO₂, Petroleum ether/Ethyl acetate=100/1 to 4/1) to afford the title compound (12) (23.1 g, 50.40 mmol, 98.58% yield) as a yellow oil. ¹H NMR (400 MHz, CDCl₃) δ 10.36 (s, 1H), 7.29 (d, J=1.6 Hz, 1H), 7.14-7.24 (m, 5H), 6.82-6.87 (m, 4H), 4.25 (s, 4H), 3.78-3.82 (m, 6H).

### 3-(bis(4-Methoxybenzyl)amino)-2-fluoro-6-(trifluoromethyl)benzaldehyde (13)

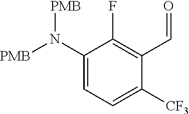

To a solution of 3-[bis[(4-methoxyphenyl)methyl]amino]-6-bromo-2-fluoro-benzaldehyde (12) (22 g, 48.00 mmol) in DMF (150 mL) was added methyl 2,2-difluoro-2-fluorosulfonyl-acetate (46.11 g, 240.01 mmol, 30.54 mL) and CuI (18.28 g, 96.00 mmol) under N₂. The mixture was stirred at 100° C. under N₂ for 2 hours. TLC showed complete conversion. The reaction mixture was diluted with water (500 mL), filtered and extracted with EtOAc (500 mL×2). The combined organic layers were dried over Na₂SO₄, filtered and concentrated under reduced pressure. The residue was purified by column chromatography (SiO₂, Petroleum ether/Ethyl acetate=30/1 to 5/1) to afford the title compound (13) (21 g, 46.94 mmol, 97.78% yield) as a yellow oil. ¹H NMR (400 MHz, CDCl₃) δ 10.44 (d, J=1.6 Hz, 1H), 7.33 (d, J=8.8 Hz, 1H), 7.17 (d, J=8.8 Hz, 4H), 6.97 (br t, J=8.4 Hz, 1H), 6.83-6.88 (m, 4H), 4.39 (s, 4H), 3.80 (s, 6H).

### Methyl 5-[3-[bis[(4-methoxyphenyl)methyl]amino]-2-fluoro-6-(trifluoromethyl)phenyl]-5-hydroxy-3-oxopentanoate (14)

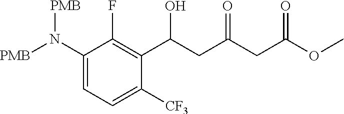

To a solution of methyl 3-oxobutanoate (15.57 g, 134.10 mmol, 14.46 mL) in THF (200 mL) was added NaH (5.36 g, 134.10 mmol, 60% purity) and n-BuLi (2.5 M, 53.64 mL) at 0° C. The mixture was stirred at 0° C. for 0.5 hour, then the mixture was cooled to −60° C., and 3-[bis[(4-methoxyphenyl)methyl]amino]-2-fluoro-6-(trifluoromethyl)benzaldehyde (13) (20 g, 44.70 mmol) in THF (100 mL) was added dropwise. The mixture was stirred at −60° C. for 0.5 hour. LCMS showed complete conversion. The reaction mixture was quenched by the addition of saturated aqueous NH₄Cl solution (30 mL), and extracted with EtOAc (30 mL×2). The combined organic layers were dried over Na₂SO₄, filtered and concentrated under reduced pressure. The residue was purified by column chromatography (SiO₂, Petroleum ether/Ethyl acetate=30/1 to 5/1) to afford the title compound (14) (20 g, 35.49 mmol, 79.40% yield) as a white solid. LCMS (ESI, m/z): 564.3 [M+H]⁺.

### Methyl 2-[3-[bis[(4-methoxyphenyl)methyl]amino]-2-fluoro-6-(trifluoromethyl)phenyl]-4-oxo-2,3-dihydropyran-5-carboxylate (15)

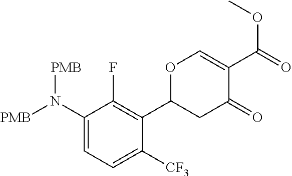

To a solution of methyl 5-[3-[bis[(4-methoxyphenyl)methyl]amino]-2-fluoro-6-(trifluoromethyl)phenyl]-5-hydroxy-3-oxo-pentanoate (14) (2.00 g, 3.55 mmol) in DCM (20 mL) was added DMF-DMA (507.49 mg, 4.26 mmol, 565.76 L), the mixture was stirred at 25° C. for 12 hours, then BF₃·Et₂O (604.45 mg, 4.26 mmol, 523.78 μL) was added. The mixture was stirred at 25° C. for 1 hour. LCMS showed complete conversion. The reaction mixture was concentrated under reduced pressure. The residue was purified by column chromatography (SiO₂, Petroleum ether/Ethyl acetate=30/1 to 5/1) to afford the title compound (15) (1.3 g, 2.27 mmol, 63.87% yield) as a white solid. LCMS (ESI, m/z): 574.3 [M+H]⁺. ¹H NMR (400 MHz, CDCl₃) δ 8.44 (s, 1H), 7.29 (d, J=8.8 Hz, 1H), 7.16 (d, J=8.8 Hz, 4H), 6.90-6.94 (m, 1H), 6.85-6.88 (m, 4H), 5.86 (dd, J=15.2, 3.2 Hz, 1H), 4.38 (s, 4H), 3.85 (s, 3H), 3.80 (s, 8H).

### Methyl 2-[3-[bis[(4-methoxyphenyl)methyl]amino]-2-fluoro-6-(trifluoromethyl)phenyl]-4-hydroxy-3,6-dihydro-2H-pyran-5-carboxylate (16)

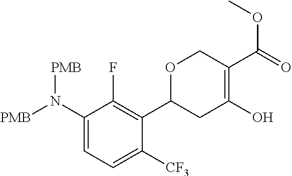

To a solution of methyl 2-[3-[bis[(4-methoxyphenyl)methyl]amino]-2-fluoro-6-(trifluoromethyl)phenyl]-4-oxo-2,3-dihydropyran-5-carboxylate (15) (1.2 g, 2.09 mmol) in THF (20 mL) was added LiBHEt₃ (1 M, 2.51 mL) at −60° C. The mixture was stirred at −60° C. for 0.5 hour. LCMS showed complete conversion. The reaction mixture was quenched by the addition of saturated aqueous NH₄Cl (30 mL), and extracted with EtOAc (30 ml×3). The combined organic layers were dried over Na₂SO₄, filtered and concentrated under reduced pressure. The residue was purified by column chromatography (SiO₂, Petroleum ether/Ethyl acetate=30/1 to 5/1) to afford the title compound (16) (600 mg, 1.04 mmol, 49.82% yield) as a white solid. LCMS (ESI, m/z): 576.3 [M+H]⁺. ¹H NMR (400 MHz, CDCl₃) δ 11.85 (s, 1H), 7.24 (br d, J=9.2 Hz, 1H), 7.17 (br d, J=8.4 Hz, 4H), 6.82-6.88 (m, 5H), 4.96 (br d, J=9.6 Hz, 1H), 4.60 (d, J=14.0 Hz, 1H), 4.28-4.40 (m, 5H), 3.80 (s, 9H), 3.04-3.15 (m, 1H), 2.41 (br d, J=17.6 Hz, 1H).

### 7-[3-[bis[(4-Methoxyphenyl)methyl]amino]-2-fluoro-6-(trifluoromethyl)phenyl]-2-methylsulfanyl-7,8-dihydro-5H-pyrano[4,3-d]pyrimidin-4-ol (17)

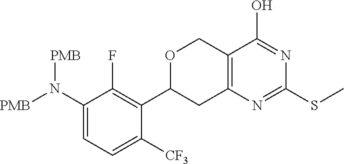

To a solution of methyl 2-[3-[bis[(4-methoxyphenyl)methyl]amino]-2-fluoro-6-(trifluoromethyl)phenyl]-4-hydroxy-3,6-dihydro-2H-pyran-5-carboxylate (16) (500 mg, 868.74 mol) and NaHCO₃ (1.46 g, 17.37 mmol, 676.05 μL) in EtOH (3 mL) and H₂O (1 mL) was added 2-methylisothiourea; sulfuric acid (2.42 g, 8.69 mmol). The mixture was stirred at 50° C. for 2 hours. LCMS showed complete conversion. The reaction mixture was filtered and concentrated under reduced pressure. The residue was purified by column chromatography (SiO₂, Petroleum ether/Ethyl acetate=30/1 to 3/1) to afford the title compound (17) (500 mg, 812.17 mol, 93.49% yield) as a white solid. LCMS (ESI, m/z): 616.4 [M+H]⁺. ¹H NMR (400 MHz, CDCl₃) δ 7.25 (s, 1H), 7.17 (br d, J=8.4 Hz, 4H), 6.78-6.88 (m, 5H), 5.01-5.06 (m, 1H), 4.93 (br d, J=15.2 Hz, 1H), 4.61 (br d, J=16.0 Hz, 1H), 4.24-4.41 (m, 5H), 3.80 (s, 6H), 3.33-3.43 (m, 1H), 2.72 (br d, J=18.0 Hz, 1H), 2.57 (s, 3H).

### 7-(3-(bis(4-Methoxybenzyl)amino)-2-fluoro-6-(trifluoromethyl)phenyl)-2-(methylthio)-7,8-dihydro-5H-pyrano[4,3-d]pyrimidin-4-yl trifluoromethanesulfonate (18)

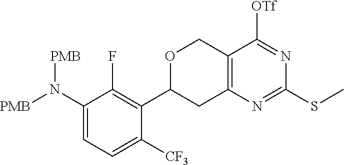

To a solution of 7-[3-[bis[(4-methoxyphenyl)methyl]amino]-2-fluoro-6-(trifluoromethyl)phenyl]-2-methylsulfanyl-7,8-dihydro-5H-pyrano[4,3-d]pyrimidin-4-ol (370 mg, 601.00 mol) (17) in DCM (5 mL) was added Tf₂O (254.35 mg, 901.51 mol, 148.74 μL) and DIEA (233.03 mg, 1.80 mmol, 314.05 μL) at 0° C. The mixture was stirred at 25° C. for 1 hour. LCMS showed clean conversion. The reaction mixture was added to a saturated aqueous NH₄Cl solution (20 mL), and extracted with EtOAc (20 mL×3). The combined organic layers were washed with brine (10 mL), dried over Na₂SO₄, filtered and concentrated under reduced pressure. The residue was purified by prep-TLC (SiO₂, Petroleum ether:Ethyl acetate=5:1) to afford the title compound (18) (330 mg, 441.35 mol, 73.44% yield) as a white solid. LCMS (ESI, m/z): 748.3 [M+H]⁺. ¹H NMR (400 MHz, CDCl₃) δ 7.29 (s, 1H), 7.17 (d, J=8.4 Hz, 4H), 6.88-6.84 (m, 5H), 5.12 (d, J=9.1 Hz, 1H), 5.02 (d, J=15.6 Hz, 1H), 4.78 (d, J=15.6 Hz, 1H), 4.41-4.34 (m, 3H), 3.80 (s, 6H), 3.59 (dd, J=11.6, 18.0 Hz, 1H), 3.03 (dd, J=3.2, 18.4 Hz, 1H), 2.56 (s, 3H).

### 5-(7-(3-(bis(4-Methoxybenzyl)amino)-2-fluoro-6-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (19)

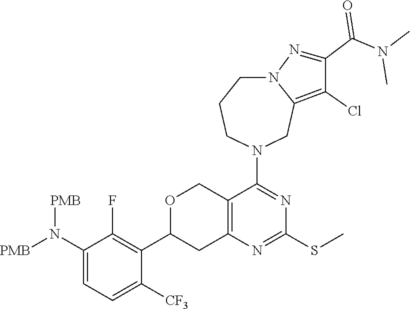

To a solution of [7-[3-[bis[(4-methoxyphenyl)methyl]amino]-2-fluoro-6-(trifluoromethyl)phenyl]-2-methylsulfanyl-7,8-dihydro-5H-pyrano[4,3-d]pyrimidin-4-yl]trifluoromethanesulfonate (18) (320 mg, 427.98 mol) in DMF (3 mL) was added DIEA (110.63 mg, 855.96 mol, 149.09 μL) and 3-chloro-N,N-dimethyl-5,6,7,8-tetrahydro-4H-pyrazolo[1,5-a][1,4]diazepine-2-carboxamide (9) (103.87 mg, 427.98 mol). The mixture was stirred at 50° C. for 1 hour. LCMS showed clean conversion. The reaction mixture was added to a saturated aqueous NH₄Cl solution (10 mL), and extracted with EtOAc (30 mL×3). The combined organic layers were washed with brine (20 mL), dried over Na₂SO₄, filtered and concentrated under reduced pressure. The residue was purified by prep-TLC (SiO₂, DCM:MeOH=20:1) to afford the title compound (19) (235 mg, 279.65 mol, 65.34% yield) as a white solid. LCMS (ESI, m/z): 840.5 [M+H]⁺. ¹H NMR (400 MHz, CDCl₃) δ 7.24 (s, 1H), 7.15 (d, J=8.8 Hz, 4H), 6.86-6.82 (m, 5H), 5.17 (dd, J=3.2, 11.2 Hz, 1H), 5.00 (d, J=14.0 Hz, 1H), 4.83 (d, J=16.8 Hz, 1H), 4.73 (d, J=14.0 Hz, 1H), 4.51-4.44 (m, 2H), 4.40-4.35 (m, 1H), 4.31 (d, J=9.6 Hz, 4H), 4.00-3.92 (m, 1H), 3.80 (s, 1H), 3.79 (s, 6H), 3.69-3.59 (m, 1H), 3.46 (dd, J=11.2, 18.0 Hz, 1H), 3.10 (s, 6H), 2.50 (s, 3H), 2.39-2.28 (m, 1H), 2.22-2.10 (m, 1H).

### 5-(7-(3-(bis(4-Methoxybenzyl)amino)-2-fluoro-6-(trifluoromethyl)phenyl)-2-(methyl sulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (20)

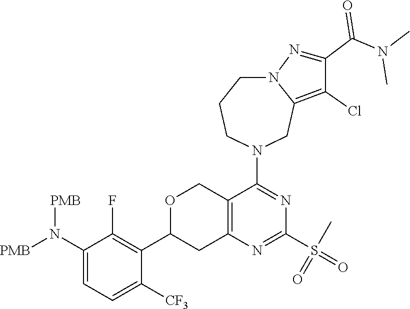

To a solution of 5-[7-[3-[bis[(4-methoxyphenyl)methyl]amino]-2-fluoro-6-(trifluoromethyl)phenyl]-2-methylsulfanyl-7,8-dihydro-5H-pyrano[4,3-d]pyrimidin-4-yl]-3-chloro-N,N-dimethyl-4,6,7,8-tetrahydropyrazolo[1,5-a][1,4]diazepine-2-carboxamide (19) (230 mg, 273.70 mol) in DCM (3 mL) was added m-CPBA (111.13 mg, 547.41 mol, 85% purity), the mixture was stirred at 25° C. for 1 hour. LCMS showed clean conversion. The pH of the mixture was adjusted to 8-9 by adding a saturated aqueous NaHCO₃ solution, and extracted with EtOAc (20 mL×3). The combined organic layers were washed with brine (20 mL), dried over Na₂SO₄, filtered and concentrated under reduced pressure. The residue was purified by prep-TLC (SiO₂, DCM:MeOH=20:1) to afford the title compound (20) (115 mg, 105.47 mol, 38.53% yield, 80% purity) as a white solid. LCMS (ESI, m/z): 872.5 [M+H]⁺.

### 5-(7-(3-(bis(4-Methoxybenzyl)amino)-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (21)

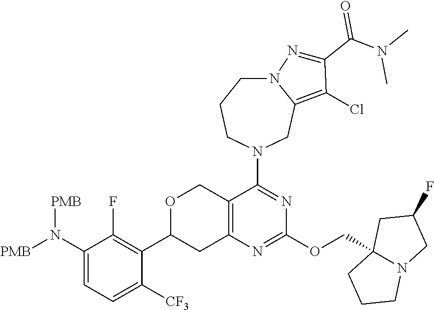

To a solution of [(2R,8S)-2-fluoro-1,2,3,5,6,7-hexahydropyrrolizin-8-yl]methanol (40.15 mg, 252.20 mol) in THF (2 mL) was added t-BuONa (36.36 mg, 378.30 mol) and 5-[7-[3-[bis[(4-methoxyphenyl)methyl]amino]-2-fluoro-6-(trifluoromethyl)phenyl]-2-methylsulfonyl-7,8-dihydro-5H-pyrano[4,3-d]pyrimidin-4-yl]-3-chloro-N,N-dimethyl-4,6,7,8-tetrahydropyrazolo[1,5-a][1,4]diazepine-2-carboxamide (20) (110 mg, 126.10 mol). The mixture was stirred at 25° C. for 1 hour. LCMS showed clean conversion. The reaction mixture was added to H₂O (10 mL), and extracted with EtOAc (10 mL×3). The combined organic layers were washed with brine (10 mL), dried over Na₂SO₄, filtered and concentrated under reduced pressure to afford the title compound (21) (100 mg, crude) as a yellow oil. LCMS (ESI, m/z): 951.7 [M+H]⁺.

### 5-(7-(3-Amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (22)

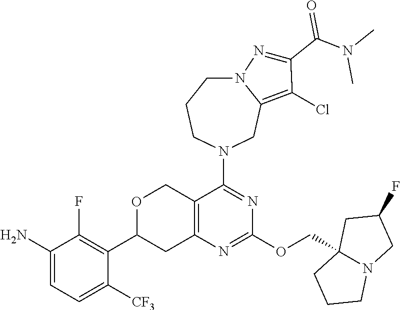 [SMILES: CN(C)C(=O)c1nn2c(c1Cl)CN(c1nc(OC[C@@]34CCCN3C[C@H](F)C4)nc3c1COC(c1c(C(F)(F)F)ccc(N)c1F)C3)CCC2]

To a solution of 5-[7-1[3-[bis[(4-methoxyphenyl)methyl]amino]-2-fluoro-6-(trifluoromethyl)phenyl]-2-[[(2R,8S)-2-fluoro-1,2,3,5,6,7-hexahydropyrrolizin-8-yl]methoxy]-7,8-dihydro-5H-pyrano[4,3-d]pyrimidin-4-yl]-3-chloro-N,N-dimethyl-4,6,7,8-tetrahydropyrazolo[1,5-a][1,4]diazepine-2-carboxamide (21) (100 mg, 105.11 mol) in DCM (1 mL) was added TFA (767.50 mg, 6.73 mmol, 0.5 mL). The mixture was stirred at 25° C. for 30 min. LCMS showed clean conversion. The pH of the mixture was adjusted to 8-9 by adding saturated aqueous NaHCO₃ solution, and extracted with EtOAc (10 mL×3). The combined organic layers were washed with brine (10 mL), dried over Na₂SO₄, filtered and concentrated under reduced pressure. The residue was purified by prep-TLC (SiO₂, DCM:MeOH=10:1) to give the crude product, and then purified by prep-HPLC (column: C18 Waters Xbridge 150×25 mm, 5 m; mobile phase: [H₂O w/0.1% NH₄HCO₃-MeCN]; B %: gradient: 38%-68 over 9 min) to afford the title compound (22) (19.24 mg, 26.79 μmol, 25.48% yield, 99% purity) as a white solid. LCMS (ESI, m/z): 711.3 [M+H]⁺. ¹H NMR (400 MHz, CDCl₃) δ 7.29 (s, 1H), 6.77 (t, J=8.4 Hz, 1H), 5.44-5.20 (m, 1H), 5.13 (dd, J=4.4, 12.0 Hz, 1H), 5.05-4.97 (m, 1H), 4.85-4.77 (m, 1H), 4.72 (br d, J=13.2 Hz, 1H), 4.55-4.44 (m, 2H), 4.41-4.30 (m, 1H), 4.10 (s, 4H), 3.99-3.91 (m, 1H), 3.69-3.60 (m, 1H), 3.44-3.32 (m, 2H), 3.30-3.17 (m, 2H), 3.10 (s, 6H), 3.05-2.91 (m, 2H), 2.34-2.19 (m, 5H), 2.04-1.85 (m, 3H).

Racemic 5-(7-(3-amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide was separated by chiral SFC to afford arbitrarily assigned.

### 5-((S)-7-(3-Amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 1)

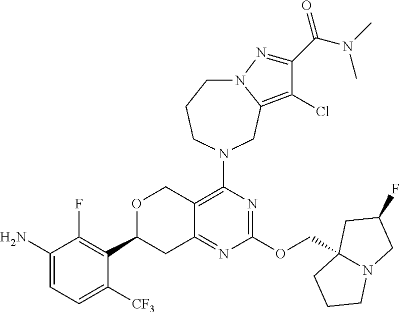 [SMILES: CN(C)C(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2]

5-((S)-7-(3-amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (peak 1, retention time=1.153 min) (5.52 mg, 7.25 mol, 32.84% yield, 93.4% purity) was obtained by chiral SFC separation as an off-white solid. LCMS (ESI, m/z): 711.3 [M+H]⁺. ¹H NMR (400 MHz, CDCl₃) δ 7.29 (s, 1H), 6.77 (t, J=8.4 Hz, 1H), 5.44-5.21 (m, 1H), 5.17-5.06 (m, 1H), 4.99 (d, J=13.2 Hz, 1H), 4.81-4.67 (m, 2H), 4.55-4.45 (m, 2H), 4.39-4.29 (m, 1H), 4.20-4.05 (m, 4H), 3.99-3.89 (m, 1H), 3.66-3.58 (m, 1H), 3.46-3.35 (m, 2H), 3.34-3.19 (m, 2H), 3.10 (d, J=2.0 Hz, 6H), 3.03-2.92 (m, 2H), 2.35-2.16 (m, 5H), 2.05 (s, 3H).

### 5-((S)-7-(3-Amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (23)

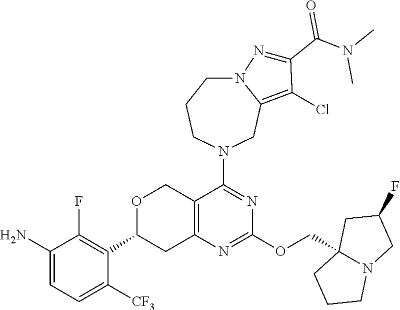 [SMILES: CN(C)C(=O)c1nn2c(c1Cl)CN(c1nc(OC[C@@]34CCCN3C[C@H](F)C4)nc3c1CO[C@@H](c1c(C(F)(F)F)ccc(N)c1F)C3)CCC2]

5-((S)-7-(3-amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (peak 2, retention time=1.785 min), (6.44 mg, 8.17 mol, 37.00% yield, 90.2% purity) was obtained by chiral SFC separation as a yellow solid. LCMS (ESI, m/z): 711.3 [M+H]⁺. ¹H NMR (400 MHz, CDCl₃) δ 7.29 (s, 1H), 6.77 (t, J=8.4 Hz, 1H), 5.41-5.19 (m, 1H), 5.12 (dd, J=2.8, 10.4 Hz, 1H), 5.00 (d, J=13.6 Hz, 1H), 4.81 (d, J=16.8 Hz, 1H), 4.71 (d, J=13.6 Hz, 1H), 4.54-4.42 (m, 2H), 4.40-4.29 (m, 1H), 4.21-4.01 (m, 4H), 3.99-3.88 (m, 1H), 3.70-3.58 (m, 1H), 3.42-3.34 (m, 2H), 3.31-3.17 (m, 2H), 3.10 (d, J=2.4 Hz, 6H), 3.02-2.90 (m, 2H), 2.31-2.17 (m, 5H), 1.89 (s, 3H).

### 3-((S)-4-(3-Chloro-2-(methylsulfonyl)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-7,8-dihydro-5H-pyrano[4,3-d]pyrimidin-7-yl)-2-fluoro-4-(trifluoromethyl)aniline (Example 211a)

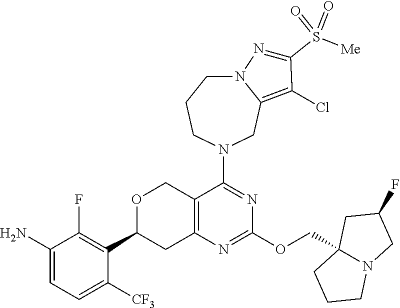 [SMILES: CS(=O)(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2]

The title compound was prepared analogously to Example 1, where 3-chloro-N,N-dimethyl-5,6,7,8-tetrahydro-4H-pyrazolo[1,5-a][1,4]diazepine-2-carboxamide was replaced with 3-chloro-2-(methylsulfonyl)-5,6,7,8-tetrahydro-4H-pyrazolo[1,5-a][1,4]diazepine. The title compound was isolated as a white solid after purification of the mixture of 3-((R)-4-(3-chloro-2-(methylsulfonyl)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-7,8-dihydro-5H-pyrano[4,3-d]pyrimidin-7-yl)-2-fluoro-4-(trifluoromethyl)aniline and 3-((S)-4-(3-chloro-2-(methylsulfonyl)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-7,8-dihydro-5H-pyrano[4,3-d]pyrimidin-7-yl)-2-fluoro-4-(trifluoromethyl)aniline by SFC (WHELK-01 250 nm*25 mm, 10 m; isocratic elution using 45% isopropanol (containing 0.1% of NH₄OH) in CO₂). MS (ESI) m/z: 718.2[M+H]⁺. Analytical SFC: Retention time=1.0 minutes. Chiralpak IK-3 50*4.6 mm, ID=3 μm, temperature=35° C. Solvent: 50% ethanol (containing 0.05% of diethyl amine) in CO₂; flow rate=3 mL/min; detector=PDA. ¹H NMR (400 MHz, CDCl₃) δ, 7.30 (s, 1H), 6.78 (t, J=8.4 Hz, 1H), 5.37-5.10 (m, 2H), 4.92 (br d, J=13.6 Hz, 1H), 4.82-4.68 (m, 2H), 4.64-4.51 (m, 2H), 4.45-4.35 (m, 1H), 4.11 (s, 2H), 4.04-3.92 (m, 2H), 3.92-3.85 (m, 1H), 3.70-3.61 (m, 1H), 3.37 (dd, J=11.2, 18.0 Hz, 1H), 3.23 (s, 3H), 3.16-3.11 (m, 1H), 3.01-2.91 (m, 2H), 2.25-2.08 (m, 5H), 1.96-1.81 (m, 4H), 1.26 (br t, J=3.3 Hz, 1H).

### 11-((S)-7-(3-amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-4-methyl-3,4,9,10,11,12-hexahydro-8H-[1,4]diazepino[1′,2′:1,5]pyrazolo[3,4-f][1,4]oxazepin-5(2H)-one (Example 212a)

### Step 1: 5-(tert-butyl) 2-ethyl 3-bromo-7,8-dihydro-4H-pyrazolo[1,5-a][1,4]diazepine-2,5(6H)-dicarboxylate

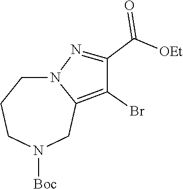 [SMILES: CCOC(=O)c1nn2c(c1Br)CN(C(=O)OC(C)(C)C)CCC2]

To a solution of 5-(tert-butyl) 2-ethyl 7,8-dihydro-4H-pyrazolo[1,5-a][1,4]diazepine-2,5(6H)-dicarboxylate (5.50 mmol) in dimethylformamide (20 mL) was added N-bromosuccinimide (6.59 mmol). After 2 hours, the reaction mixture was partitioned between water and ethyl acetate. The organic layer was separated, washed with brine, dried over sodium sulfate, filtered and concentrated under reduced pressure to afford the title compound in 98% as a white solid. This material was used in the next step without further purification. MS (ESI) m/z: 388.1[M+H]⁺.

### Step 2: 3-Bromo-5-(tert-butoxycarbonyl)-5,6,7,8-tetrahydro-4H-pyrazolo[1,5-a][1,4]diazepine-2-carboxylic acid

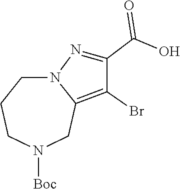 [SMILES: CC(C)(C)OC(=O)N1CCCn2nc(C(=O)O)c(Br)c2C1]

To a solution of 5-(tert-butyl) 2-ethyl 3-bromo-7,8-dihydro-4H-pyrazolo[1,5-a][1,4]diazepine-2,5(6H)-dicarboxylate (5.41 mmol) in tetrahydrofuran (10 mL), methanol (10 mL) and water (5 mL) was added lithium hydroxide monohydrate (10.8 mmol). After 2 hours, the reaction was quenched with water and the pH was adjusted to 5 by the addition of 1M hydrochloric acid solution. The resulting mixture was extracted with ethyl acetate three times, the combined organic layers were washed with brine, dried over anhydrous sodium sulfate, filtered and concentrated under reduced pressure to afford the title compound in 69% yield as a white solid. This material was used in the next step without further purification. MS (ESI) m/z: 360.1 [M+H]⁺.

### Step 3: tert-butyl 3-bromo-2-((2-hydroxyethyl)(methyl)carbamoyl)-7,8-dihydro-4H-pyrazolo[1,5-a][1,4]diazepine-5(6H)-carboxylate

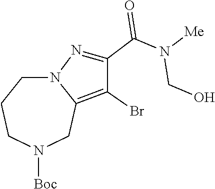 [SMILES: CN(CO)C(=O)c1nn2c(c1Br)CN(C(=O)OC(C)(C)C)CCC2]

### Step 4: tert-butyl 3-bromo-2-((2-((tert-butyldimethylsilyl)oxy)ethyl)(methyl)carbamoyl)-7,8-dihydro-4H-pyrazolo[1,5-a][1,4]diazepine-5(6H)-carboxylate

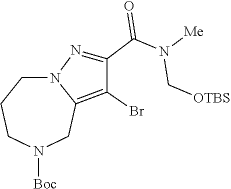 [SMILES: CN(CO[Si](C)(C)C(C)(C)C)C(=O)c1nn2c(c1Br)CN(C(=O)OC(C)(C)C)CCC2]

To a solution of tert-butyl 3-bromo-2-((2-((tert-butyldimethylsilyl)oxy)ethyl)(methyl)carbamoyl)-7,8-dihydro-4H-pyrazolo[1,5-a][1,4]diazepine-5(6H)-carboxylate (4.79 mmol) in dichloromethane (15 mL) was added tert-butylchlorodimethylsilane (5.75 mmol) and N,N-diisopropylethylamine (9.59 mmol). The mixture was stirred at 40° C. for 12 hours, cooled down to room temperature and partitioned between water and ethyl acetate. The organic phase was separated, washed with brine, dried over sodium sulfate, filtered and concentrated under reduced pressure to afford a residue that was purified by silica gel chromatography (30-45% ethyl acetate in hexanes). The title compound was isolated in 69% yield as a white solid. MS (ESI) m/z: 531.2[M+H]⁺.

### Step 5: (5-(tert-butoxycarbonyl)-2-((2-((tert-butyldimethylsilyl)oxy)ethyl)(methyl)carbamoyl)-5,6,7,8-tetrahydro-4H-pyrazolo[1,5-a][1,4]diazepin-3-yl)boronic acid

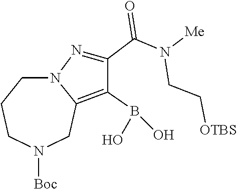 [SMILES: CN(CCO[Si](C)(C)C(C)(C)C)C(=O)c1nn2c(c1B(O)O)CN(C(=O)OC(C)(C)C)CCC2]

A solution of tert-butyl 3-bromo-2-((2-((tert-butyldimethylsilyl)oxy)ethyl)(methyl)carbamoyl)-7,8-dihydro-4H-pyrazolo[1,5-a][1,4]diazepine-5(6H)-carboxylate (3.18 mmol) and hypodiboric acid (12.7 mmol) in methanol (16 mL) and ethylene glycol (4 mL) was treated with potassium acetate (12.7 mmol) and Xphos Pd G2 (0.16 mmol). The mixture was stirred at 60° C. for 30 minutes, cooled down to room temperature and partitioned between water and ethyl acetate. The organic phase was separated, washed with brine, dried over sodium sulfate, filtered and concentrated under reduced pressure to afford a residue that was purified by preparative TLC (33% ethyl acetate in hexanes). The title compound was isolated in 8% yield as a white solid. MS (ESI) m/z: 497.3[M+H]⁺.

### Step 6: tert-butyl 2-((2-((tert-butyldimethylsilyl)oxy)ethyl)(methyl)carbamoyl)-3-hydroxy-7,8-dihydro-4H-pyrazolo[1,5-a][1,4]diazepine-5(6H)-carboxylate

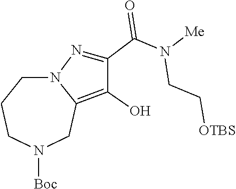 [SMILES: CN(CCO[Si](C)(C)C(C)(C)C)C(=O)c1nn2c(c1O)CN(C(=O)OC(C)(C)C)CCC2]

To a solution of (5-(tert-butoxycarbonyl)-2-((2-((tert-butyldimethylsilyl)oxy)ethyl)(methyl)carbamoyl)-5,6,7,8-tetrahydro-4H-pyrazolo[1,5-a][1,4]diazepin-3-yl)boronic acid (0.39 mmol) in tetrahydrofuran (2 mL) and water (2 mL) was added sodium carbonate (0.78 mmol) and 30% hydrogen peroxide (0.78 mmol). After 30 minutes, the reaction was quenched with water and aqueous sodium sulfite. The pH of the resulting mixture was adjusted to 6 by addition of 1M hydrochloric acid solution. The aqueous solution was extracted with ethyl acetate three times. The combined organic layers were dried over soldium sulfatem filtered and concentrated to afford a residue that was purified by preparative TLC (66% ethyl acetate in hexanes). The title compound was isolated in 40% yield as a white solid. MS (ESI) m/z: 469.3 [M+H]⁺.

### Step 7: tert-butyl 3-hydroxy-2-((2-hydroxyethyl)(methyl)carbamoyl)-7,8-dihydro-4H-pyrazolo[1,5-a][1,4]diazepine-5(6H)-carboxylate

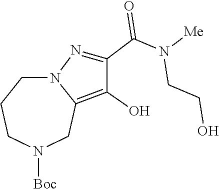 [SMILES: CN(CCO)C(=O)c1nn2c(c1O)CN(C(=O)OC(C)(C)C)CCC2]

A 1M solution of tetrabuylammonium fluoride in THF (0.24 mmol) was added over a solution of tert-butyl 2-((2-((tert-butyldimethylsilyl)oxy)ethyl)(methyl)carbamoyl)-3-hydroxy-7,8-dihydro-4H-pyrazolo[1,5-a][1,4]diazepine-5(6H)-carboxylate (0.20 mol) in THF (5 mL). After 1 hour, the reaction was quenched with water and extracted with ethyl acetate three times. The combined organic layers were dried over anhydrous sodium sulfate, filtered and concentrated under reduced pressure to afford a residue that was purified by silica gel chromatography (66% ethyl acetate in hexanes). The title compound was isolated in 76% yield as white solid. MS (ESI) m/z: 355.3 [M+H]⁺.

### Step 8: tert-butyl 4-methyl-5-oxo-2,3,4,5,9,10-hexahydro-8H-[1,4]diazepino[1′,2′:1,5]pyrazolo[3,4-f][1,4]oxazepine-11(12H)-carboxylate

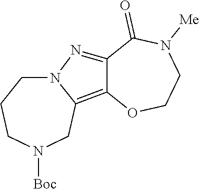 [SMILES: CN1CCOc2c(nn3c2CN(C(=O)OC(C)(C)C)CCC3)C1=O]

A solution of tert-butyl 3-hydroxy-2-((2-hydroxyethyl)(methyl)carbamoyl)-7,8-dihydro-4H-pyrazolo[1,5-a][1,4]diazepine-5(6H)-carboxylate (0.15 mmol) in tetrahydrofuran (5 mL) was treated with triphenylphosphine (0.90 mmol) and diisopropyl azodicarboxylate (0.90 mmol). After 30 minutes, the volatiles were removed under reduced pressure and the residue was purified by preparative TLC (5% methanol in ethyl acetate). The title compound was isolated in 93% yield as a white solid. MS (ESI) m/z: 337.2 [M+H]⁺.

### Step 9: 4-methyl-3,4,9,10,11,12-hexahydro-8H-[1,4]diazepino[1′,2′:1,5]pyrazolo[3,4-f][1,4]oxazepin-5(2H)-one

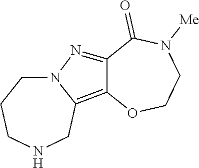 [SMILES: CN1CCOc2c(nn3c2CNCCC3)C1=O]

The title compound was prepared analogously to example 1, where tert-butyl 3-chloro-2-(dimethylcarbamoyl)-4,6,7,8-tetrahydropyrazolo[1,5-a][1,4]diazepine-5-carboxylate was replaced with tert-butyl 4-methyl-5-oxo-2,3,4,5,9,10-hexahydro-8H-[1,4]diazepino[1′,2′:1,5]pyrazolo[3,4-f][1,4]oxazepine-11(12H)-carboxylate. The title compound was isolated as a white solid. MS (ESI) m/z: 237.1 [M+H]⁺.

### Step 10: 11-((S)-7-(3-amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-4-methyl-3,4,9,10,11,12-hexahydro-8H-[1,4]diazepino[1′,2′:1,5]pyrazolo[3,4-f][1,4]oxazepin-5(2H)-one

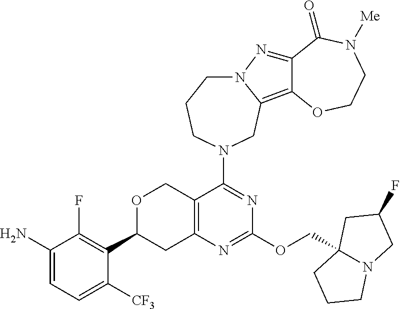 [SMILES: CN1CCOc2c(nn3c2CN(c2nc(OC[C@@]45CCCN4C[C@H](F)C5)nc4c2CO[C@H](c2c(C(F)(F)F)ccc(N)c2F)C4)CCC3)C1=O]

The title compound was prepared analogously to Example 1, where 3-chloro-N,N-dimethyl-5,6,7,8-tetrahydro-4H-pyrazolo[1,5-a][1,4]diazepine-2-carboxamide was replaced with 4-methyl-3,4,9,10,11,12-hexahydro-8H-[1,4]diazepino[1′,2′:1,5]pyrazolo[3,4-f][1,4]oxazepin-5(2H)-one. MS (ESI) m/z: 705.3 [M+H]⁺.

### 5-((S)-7-(3-amino-5-chloro-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 213a)

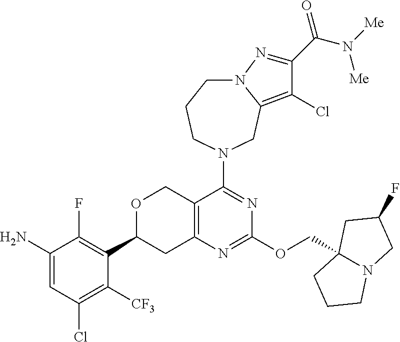 [SMILES: CN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2]

The title compound was prepared analogously to Example 1, where 4-bromo-2-fluoro-aniline was replaced with 4-bromo-5-chloro-2-fluoroaniline. The title compound was isolated as a white solid after purification of the mixture of 5-((R)-7-(3-amino-5-chloro-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((S)-7-(3-amino-5-chloro-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralpak IF 250 nm*30 mm, 10 m; isocratic elution using 50% CH₃CN/methanol (containing 0.1% of NH₄OH) in CO₂). MS (ESI) m/z: 745.2[M+H]⁺. Analytical SFC: Retention time=1.62 minutes. Chiralpak IF-3 50*4.6 mm, ID=3 μm, temperature=35° C. Solvent: 40% methanol (containing 0.05% of diethyl amine) in CO₂; flow rate=3 mL/min; detector=PDA. ¹H NMR (400 MHz, CDCl₃) δ 6.84 (d, J=8.0 Hz, 1H), 5.35-5.21 (m, 1H), 5.20-5.19 (m, 2H), 4.96 (br d, J=13.2 Hz, 1H), 4.81 (br d, J=16.4 Hz, 1H), 4.69 (d, J=13.6 Hz, 1H), 4.55-4.40 (m, 1H), 4.38-4.31 (m, 1H), 4.18 (s, 2H), 4.03 (s, 2H), 3.96-3.89 (m, 1H), 3.68-3.59 (m, 1H), 3.37-3.14 (m, 4H), 3.10 (s, 6H), 3.02-2.94 (m, 2H), 2.24 (br d, J=1.6 Hz, 2H), 2.20-2.10 (m, 3H), 1.99-1.84 (m, 3H).

### 5-((S)-7-(3-amino-2,5-difluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 214a)

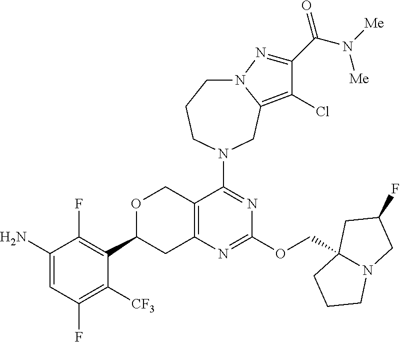 [SMILES: CN(C)C(=O)c1nn2c(c1Cl)CN(c1nc(OC[C@@]34CCCN3C[C@H](F)C4)nc3c1CO[C@H](c1c(F)c(N)cc(F)c1C(F)(F)F)C3)CCC2]

The title compound was prepared analogously to Example 1, where 4-bromo-2-fluoro-aniline was replaced with 4-bromo-2,5-difluoroaniline. The title compound was isolated as a white solid after purification of the mixture of 5-((R)-7-(3-amino-2,5-difluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((S)-7-(3-amino-2,5-difluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralcel OJ 250 nm*30 mm, m; isocratic elution using 20% methanol (containing 0.1% of NH₄OH) in CO₂). MS (ESI) m/z: 729.3 [M+H]+. Analytical SFC: Retention time=1.51 minutes. Chiralcel OJ-3 50*4.6 mm, ID=3 μm, temperature=35° C. Solvent: 5-40% methanol (containing 0.05% of diethyl amine) in CO₂; flow rate=3 mL/min; total run-time=3.00 minutes; detector=PDA. ¹H NMR (400 MHz, CDCl₃) δ 6.50 (dd, J=7.2, 12.4 Hz, 1H), 5.36-5.17 (m, 1H), 5.17-5.10 (m, 1H), 4.97 (br d, J=13.6 Hz, 1H), 4.81 (d, J=16.4 Hz, 1H), 4.70 (d, J=13.6 Hz, 1H), 4.56-4.40 (m, 2H), 4.34 (br dd, J=10.4, 12.8 Hz, 1H), 4.27 (s, 2H), 4.02 (s, 2H), 3.97-3.86 (m, 1H), 3.64 (ddd, J=3.2, 10.4, 13.7 Hz, 1H), 3.39-3.16 (m, 3H), 3.10 (s, 6H), 3.01-2.91 (m, 2H), 2.37-2.07 (m, 5H), 1.98-1.79 (m, 3H).

### 5-((S)-7-(3-amino-2-fluoro-5-methyl-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 215a)

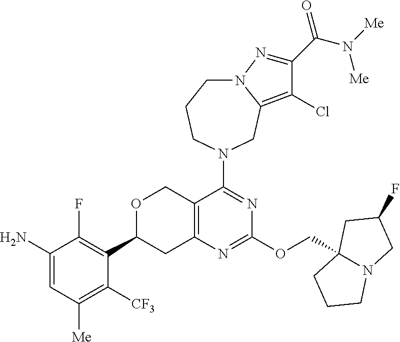 [SMILES: Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F]

The title compound was prepared analogously to Example 1, where 4-bromo-2-fluoro-aniline was replaced with 4-bromo-2-fluoro-5-methyl-aniline. The title compound was isolated as a white solid after purification of the mixture of 5-((R)-7-(3-amino-2-fluoro-5-methyl-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((S)-7-(3-amino-2-fluoro-5-methyl-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralpak IK 250 nm*30 mm, 10 m; isocratic elution using 70% acetonitrile/isopropanol (containing 0.1% of NH₄OH) in CO₂). MS (ESI) m/z: 725.3 [M+H]+. Analytical SFC: Retention time=1.28 minutes. Chiralpak IK-3 50*4.6 mm, ID=3 μm, temperature=35° C. Solvent: 40% acetonitrile/isopropanol (containing 0.05% of diethyl amine) in CO₂; flow rate=3 mL/min; detector=PDA. ¹H NMR (400 MHz, CDCl₃) δ 6.8 (d, J=8.4 Hz, 1H), 5.37-5.13 (m, 2H), 4.97 (br d, J=13.6 Hz, 1H), 4.81 (br d, J=16.4 Hz, 1H), 4.69 (d, J=13.6 Hz, 1H), 4.58-4.38 (m, 2H), 4.34 (br dd, J=10.4, 12.8 Hz, 1H), 4.02 (br d, J=14.8 Hz, 4H), 3.95-3.83 (m, 1H), 3.63 (ddd, J=3.2, 10.4, 14.0 Hz, 1H), 3.36 (dd, J=12.0, 18.0 Hz, 1H), 3.31-3.17 (m, 2H), 3.15 (br s, 1H), 3.10 (s, 6H), 3.01-2.93 (m, 2H), 2.39 (q, J=4.0 Hz, 3H), 2.34-2.21 (m, 2H), 2.20-2.04 (m, 3H), 1.98-1.82 (m, 3H).

### 5-((S)-7-(3-amino-2,4-difluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 216a)

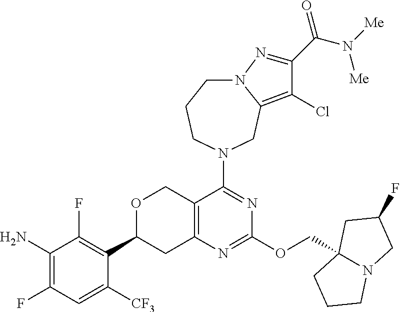 [SMILES: CN(C)C(=O)c1nn2c(c1Cl)CN(c1nc(OC[C@@]34CCCN3C[C@H](F)C4)nc3c1CO[C@H](c1c(C(F)(F)F)cc(F)c(N)c1F)C3)CCC2]

The title compound was prepared analogously to Example 1, where 4-bromo-2-fluoro-aniline was replaced with 4-bromo-2,5-difluoro-aniline. The title compound was isolated as a white solid after purification of the mixture of 5-((R)-7-(3-amino-2,4-difluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((S)-7-(3-amino-2,4-difluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (Welch Ultimate XB-CN 250 nm*50 mm, 10 m; 1-30% ethanol/hexanes (containing 1% of formic acid) in CO₂). Run-time=15 minutes. MS (ESI) m/z: 729.3 [M+H]+. Analytical SFC: Retention time=2.00 minutes. Chiralpak IH-3 50*4.6 mm, ID=3 μm, temperature=35° C. Solvent: 5-40% ethanol (containing 0.05% of diethyl amine) in CO₂; flow rate=3 mL/min; run-time=3.00 minutes; detector=PDA. ¹H NMR (400 MHz, CDCl₃) δ ppm 1.83-1.94 (m, 3H) 2.08-2.18 (m, 3H) 2.20-2.25 (m, 1H) 2.28-2.37 (m, 1H) 2.89-3.00 (m, 2H) 3.10 (s, 6H) 3.12-3.19 (m, 1H) 3.19-3.28 (m, 2H) 3.33 (brdd, J=18.0, 11.6 Hz, 1H) 3.58-3.70 (m, 1H) 3.94 (brd, J=14.4 Hz, 1H) 3.98-4.08 (m, 2H) 4.13 (s, 2H) 4.29-4.40 (m, 1H) 4.41-4.56 (m, 2H) 4.70 (d, J=13.6 Hz, 1H) 4.82 (br d, J=16.4 Hz, 1H) 4.99 (br d, J=13.6 Hz, 1H) 5.10 (br dd, J=11.2, 3.2 Hz, 1H) 5.17-5.39 (m, 1H) 7.19 (br d, J=10.4 Hz, 1H).

### 5-(7-(6-amino-3-(trifluoromethyl)pyridin-2-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 217)

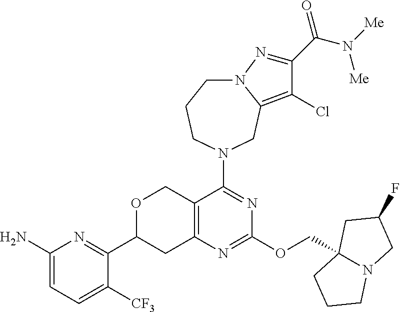 [SMILES: CN(C)C(=O)c1nn2c(c1Cl)CN(c1nc(OC[C@@]34CCCN3C[C@H](F)C4)nc3c1COC(c1nc(N)ccc1C(F)(F)F)C3)CCC2]

The title compound was prepared analogously to Example 1, where 4-bromo-2-fluoro-aniline was replaced with 2-bromo-6-fluoropyridine. The title compound was isolated as a white solid. MS (ESI) m/z: 694.3 [M+H]+. ¹H NMR (400 MHz, methanol-d₄) δ 7.68 (d, J=8.8 Hz, 1H), 6.56 (d, J=8.8 Hz, 1H), 5.36-5.22 (m, 1H), 5.11-5.06 (m, 2H), 4.80-4.72 (m, 3H), 4.50-4.45 (m, 1H), 4.38-4.29 (m, 1H), 4.13 (t, J=10.0 Hz, 1H), 3.99-3.96 (m, 2H), 3.79-3.74 (m, 1H), 3.40-3.35 (m, 1H), 3.27-3.15 (m, 3H), 3.11-3.08 (m, 6H), 3.02-2.96 (m, 1H), 2.75 (dd, J=18.0, 3.6 Hz, 1H), 2.38-2.05 (m, 5H), 1.98-1.81 (m, 3H).

### 5-(7-(5-Amino-2-(trifluoromethyl)phenyl)-2-(((2R,7aS)-2-fluorotetrahydro-1H-pyrrolizi n-7a(5H)-yl)methoxy)-7,8-dihydro-5H-pyrano[4,3-d]pyrimidin-4-yl)-3-chloro-N,N-dime thyl-5,6,7,8-tetrahydro-4H-pyrazolo[1,5-a][1,4]diazepine-2-carboxamide (Example 230)

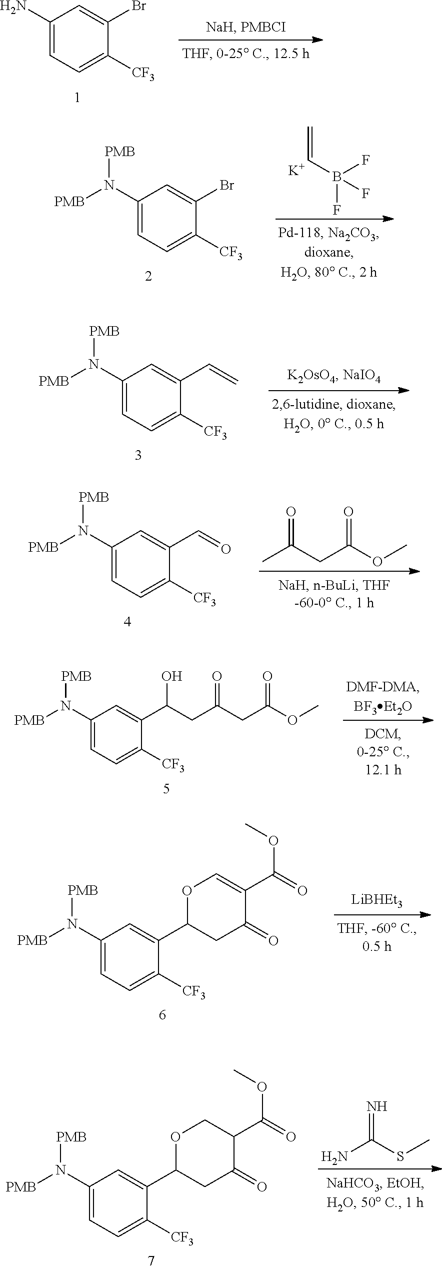

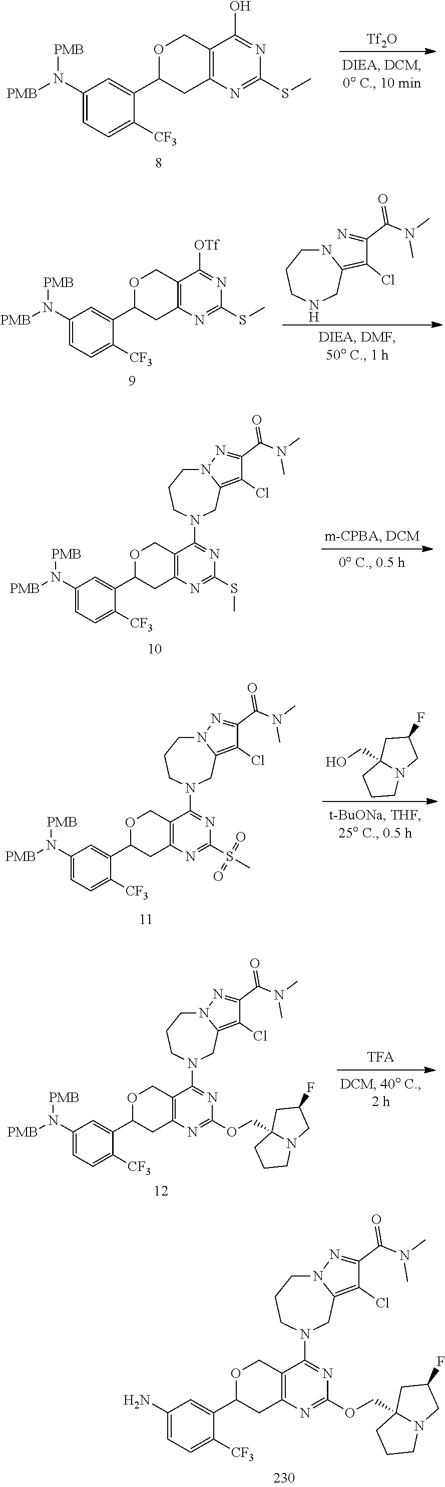

### Step 1: 3-bromo-N,N-bis(4-methoxybenzyl)-4-(trifluoromethyl)aniline

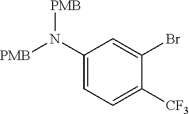

A mixture of 3-bromo-4-(trifluoromethyl)aniline (15 g, 62.49 mmol, 1.0 eq) in tetrahydrofuran (300 mL) was degassed and purged with nitrogen for three times, then to the mixture was slowly added sodium hydride (6.25 g, 156.24 mmol, 60% purity, 2.5 eq) at 0° C. and stirred at 0° C. for 0.5 h under nitrogen. Then p-methoxybenzyl chloride (23.49 g, 149.99 mmol, 20.3 mL, 2.4 eq) was added dropwise at 0° C. and stirred at 25° C. for 12 h. The reaction mixture was quenched by the addition of saturated aqueous ammonium chloride (1500 mL) and extracted with ethyl acetate (150 mL×2). The combined organic layers were washed with brine (300 mL×2), dried over anhydrous sodium sulfate, filtered and concentrated in vacuum to give a residue which was purified by flash silica gel chromatography (Petroleum ether/Ethyl acetate=10/1 to 5/1) to afford the title compound (21 g, 34.98 mmol, 56% yield, 80% purity) as a red solid. ¹H NMR (400 MHz, DMSO-d₆) δ: 7.46 (d, J=8.8 Hz, 1H), 7.17 (d, J=8.8 Hz, 4H), 7.02 (d, J=2.8 Hz, 1H), 6.91 (d, J=8.8 Hz, 4H), 6.78-6.72 (m, 1H), 4.69 (s, 4H), 3.73 (s, 6H).

### Step 2: N,N-bis(4-methoxybenzyl)-4-(trifluoromethyl)-3-vinylaniline

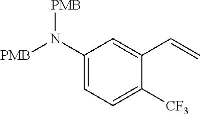

A mixture of 3-bromo-N,N-bis(4-methoxybenzyl)-4-(trifluoromethyl)aniline (20 g, 41.64 mmol, 1.0 eq), potassium trifluoro(vinyl)borate (8.36 g, 62.44 mmol, 1.5 eq), [1,1′-Bis(di-tert-butylphosphino)ferrocene]palladium(II) dichloride (2.7 g, 4.16 mmol, 0.1 eq), sodium carbonate (13.24 g, 124.92 mmol, 3.0 eq) in dioxane (300 mL) and water (60 mL) was degassed and purged with nitrogen for three times, and then the mixture was stirred at 80° C. for 2 h under nitrogen. LCMS showed the reaction was completed. The reaction mixture was quenched by water (400 mL) and extracted with ethyl acetate (200 mL×3). The combined organic layers were washed with brine (300 mL×2), dried over anhydrous sodium sulfate, filtered and concentrated under reduced pressure to give a residue which was purified by flash silica gel chromatography (Petroleum ether/Ethyl acetate=10/1 to 5/1) to afford the title compound (17.2 g, 31.39 mmol, 75% yield, 78% purity) as a brown oil. MS (ESI, m/z): 428.1[M+H]⁺. ¹H NMR (400 MHz, DMSO-d₆) δ: 7.37 (d, J=8.8 Hz, 1H), 7.20 (d, J=8.8 Hz, 4H), 6.95 (d, J=2.8 Hz, 1H), 6.92-6.89 (m, 4H), 6.88-6.82 (m, 1H), 6.71 (dd, J=2.4, 8.8 Hz, 1H), 5.59 (d, J=17.6 Hz, 1H), 5.36-5.28 (m, 1H), 4.72 (s, 4H), 3.72 (s, 6H).

### Step 3: 5-(bis(4-methoxybenzyl)amino)-2-(trifluoromethyl)benzaldehyde

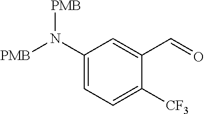

A mixture of N,N-bis[(4-methoxyphenyl)methyl]-4-(trifluoromethyl)-3-vinyl-aniline (8.2 g, 19.81 mmol, 1.0 eq), potassium osmate(vi) dihydrate (706 mg, 1.91 mmol, 0.1 eq), 2,6-dimethylpyridine (4.12 g, 38.36 mmol, 4.46 mL, 2.0 eq) in dioxane (120 mL) and water (20 mL) was degassed and purged with nitrogen for three times, then to the mixture was slowly added sodium periodate (16.42 g, 76.74 mmol, 4.26 mL, 4.0 eq) at 0° C. The reaction mixture was stirred at 0° C. for 0.5 h, then was quenched by saturated aqueous sodium sulfite (300 mL) and extracted with ethyl acetate (60 mL×3). The combined organic layers were washed with hydrochloric acid (1N, 400 mL×2) and brine (200 mL×2), dried over anhydrous sodium sulfate, filtered and concentrated in vacuum. The residue was purified by flash silica gel chromatography (Petroleum ether/Ethyl acetate=1/0 to 10/1) to afford the desired product (5.1 g, 11.88 mmol, 62% yield) as a yellow oil.

### Step 4: Methyl 5-(5-(bis(4-methoxybenzyl)amino)-2-(trifluoromethyl)phenyl)-5-hydroxy-3-oxopentanoate

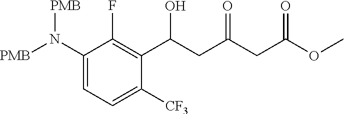

To a mixture of methyl 3-oxobutanoate (3.45 g, 29.69 mmol, 3.2 mL, 2.5 eq) in tetrahydrofuran (150 mL) was added sodium hydride (1.19 g, 29.69 mmol, 60% purity, 2.5 eq) at 0° C. and the resultant reaction mixture was stirred at 0° C. for 10 min. Then n-butyllithium (2.5 M, 11.9 mL, 2.5 eq) was added and stirred at −15° C. for 20 min. Finally, 5-(bis(4-methoxybenzyl)amino)-2-(trifluoromethyl) benzaldehyde (5.1 g, 11.88 mmol, 1.0 eq) was added and stirred at −60° C. for another 30 min. The reaction mixture was quenched by the addition of saturated aqueous ammonium chloride (200 mL) and extracted with ethyl acetate (40 mL×2). The combined organic layers were washed with brine (60 mL×2), dried over anhydrous sodium sulfate, filtered and concentrated in vacuum to give a residue which was purified by flash silica gel chromatography (PE/EA=10/1 to 3/1) to afford the desired product (2.2 g, 4.03 mmol, 34% yield) as a light yellow oil.

### Step 5: methyl 2-(5-(bis(4-methoxybenzyl)amino)-2-(trifluoromethyl)phenyl)-4-oxo-3,4-dihydro-2H-pyran-5-carboxylate

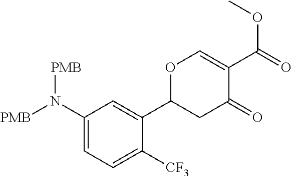

To a mixture of methyl 5-(5-(bis(4-methoxybenzyl) amino)-2-(trifluoromethyl)phenyl)-5-hydroxy-3-oxopentanoate (2.2 g, 4.03 mmol, 1.0 eq) in dichloromethane (25 mL) was added N,N-dimethyl-formamide dimethyl acetal (961 mg, 8.07 mmol, 1.1 mL, 2.0 eq). The resultant mixture was stirred at 25° C. for 12 h. Then boron trifluoride-ethyl complex (1.03 g, 7.26 mmol, 0.9 mL, 1.8 eq) was added at 0° C. and stirring continued at 0° C. for 10 min. LCMS showed the reaction was completed. The reaction mixture was quenched by saturated aqueous sodium bicarbonate (30 mL) and extracted with dichloromethane (20 mL×3). The combined organic layers were washed with brine (40 mL×2), dried over anhydrous sodium sulfate, filtered and concentrated in vacuum to give the crude product (2.2 g, crude) as a yellow oil. LCMS (ESI, m/z): 556.2[M+H]⁺.

### Step 6: methyl 6-(5-(bis(4-methoxybenzyl)amino)-2-(trifluoromethyl)phenyl)-4-oxotetrahydro-2H-pyran-3-carboxylate

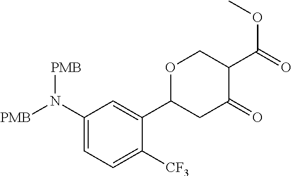

To a mixture of methyl 2-(5-(bis(4-methoxybenzyl)amino)-2-(trifluoromethyl)phenyl)-4-oxo-3,4-dihydro-2H-pyran-5-carboxylate (2.2 g, 3.96 mmol, 1.0 eq) in tetrahydrofuran (30 mL) was slowly added lithium triethylborohydride (1 M, 4.7 mL, 1.2 eq) at −65° C. The mixture was stirred at −65° C. for 0.5 h. LCMS showed the reaction was completed. The reaction mixture was quenched by saturated aqueous ammonium chloride (50 mL) and extracted with ethyl acetate (40 mL×3). The combined organic layers were washed with brine (40 mL×2), dried over anhydrous sodium sulfate, filtered and concentrated in vacuum to give a residue which was purified by flash silica gel chromatography (PE/EA=1/0 to 10/1) to afford the desired product (376 mg, 0.67 mmol, 17% yield) as a colorless oil. LCMS (ESI, m/z): 558.2 [M+H]⁺.

### Step 7: 7-(5-(bis(4-methoxybenzyl)amino)-2-(trifluoromethyl)phenyl)-2-(methylthio)-7,8-dihydro-5H-pyrano[4,3-d]pyrimidin-4-ol

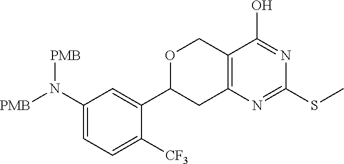

To a mixture of methyl 6-(5-(bis(4-methoxybenzyl)amino)-2-(trifluoromethyl)phenyl)-4-oxotetrahydro-2H-pyran-3-carboxylate (376 mg, 0.67 mmol, 1.0 eq), methyl carbamimidothioate (607 mg, 6.75 mmol, 10.0 eq) in ethanol (10 mL) and water (2 mL) was added sodium hydrogencarbonate (1.18 g, 13.49 mmol, 0.5 mL, 20.0 eq). The resultant mixture was stirred at 50° C. for 1 h. LCMS showed the reaction was completed. The reaction mixture was quenched by water (20 mL) and extracted with ethyl acetate (15 mL×3). The combined organic layers were washed with brine (20 mL×2), dried over anhydrous sodium sulfate, filtered and concentrated in vacuum to give the crude product (370 mg, crude) as a white solid. LCMS (ESI, m/z): 598.3[M+H]⁺.

### Step 8: 7-(5-(bis(4-methoxybenzyl)amino)-2-(trifluoromethyl)phenyl)-2-(methylthio)-7,8-dihydro-5H-pyrano[4,3-d]pyrimidin-4-yl trifluoromethanesulfonate

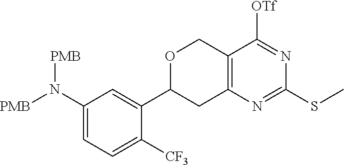

To a mixture of 7-(5-(bis(4-methoxybenzyl)amino)-2-(trifluoromethyl)phenyl)-2-(methylthio)-7,8-dihydro-5H-pyrano[4,3-d]pyrimidin-4-ol (370 mg, 0.61 mmol, 1.0 eq) in dichloromethane (3 mL) was added N,N-diisopropylethylamine (400 mg, 3.10 mmol, 0.53 mL, 5.0 eq). Then trifluoromethylsulfonic anhydride (349 mg, 1.24 mmol, 0.2 mL, 2.0 eq) was added at 0° C. and the stirring was continued at 0° C. for 10 min. LCMS showed the reaction was completed. The reaction mixture was quenched by saturated aqueous ammonium chloride (20 mL) and extracted with dichloromethane (20 mL×3). The combined organic layers were washed with brine (20 mL×2), dried over anhydrous sodium sulfate, filtered and concentrated in vacuum to give a residue which was purified by Prep-TLC (PE/EA=1/0 to 10/1) to afford the desired product (140 mg, 0.19 mmol, 31% yield) as a yellow oil. LCMS (ESI, m/z): 730.2[M+H]⁺.

### Step 9: 5-(7-(5-(bis(4-methoxybenzyl)amino)-2-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

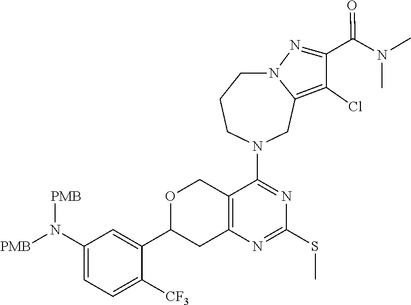

To a mixture of 3-chloro-3-chloro-N,N-dimethyl-5,6,7,8-tetrahydro-4H-pyrazolo[1,5-a][1,4]diazepine-2-carboxamide (64 mg, 0.23 mmol, 1.2 eq, hydrochloride) in N,N-dimethylformamide (2 mL) was added N,N-diisopropylethylamine (74 mg, 0.57 mmol, 0.10 mL, 3.00 eq). Then 7-(5-(bis(4-methoxybenzyl)amino)-2-(trifluoromethyl) phenyl)-2-(methylthio)-7,8-dihydro-5H-pyrano[4,3-d]pyrimidin-4-yl trifluoromethanesulfonate (140 mg, 0.19 mmol, 1.0 eq) was added and the stirring was continued at 50° C. for 1 h. LCMS showed the reaction was completed. The reaction mixture was quenched by water (10 mL) and extracted with ethyl acetate (10 mL×3). The combined organic layers were washed with brine (20 mL×2), dried over anhydrous sodium sulfate, filtered and concentrated in vacuum to give a residue which was purified by Prep-TLC (dichloromethane/methanol=10/1) to afford the desired product (85 mg, 0.10 mmol, 54% yield) as a light yellow solid. LCMS (ESI, m/z): 822.3[M+H]⁺.

### Step 10: 5-(7-(5-(bis(4-methoxybenzyl)amino)-2-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

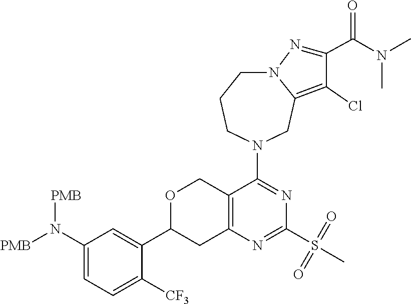

To a mixture of 5-(7-(5-(bis(4-methoxybenzyl)amino)-2-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (85 mg, 0.10 mmol, 1.0 eq) in dichloromethane (2 mL) was added 3-chloro-benzenecarboperoxoic acid (46 mg, 0.22 mmol, 85% purity, 2.2 eq) at 0° C. and stirred at 0° C. for 0.5 h. LCMS showed the reaction was completed. The reaction mixture was quenched by saturated aqueous sodium bicarbonate (15 mL) and saturated aqueous sodium sulfite (15 mL), the aqueous phase was extracted with dichloromethane (20 mL×2). The combined organic layers were washed with brine (20 mL×2), dried over anhydrous sodium sulfate, filtered and concentrated in vacuum to give the crude product (85 mg, crude) as a light yellow solid. LCMS (ESI, m/z): 853.3[M]⁺.

### Step 11: 5-(7-(5-(bis(4-methoxybenzyl)amino)-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

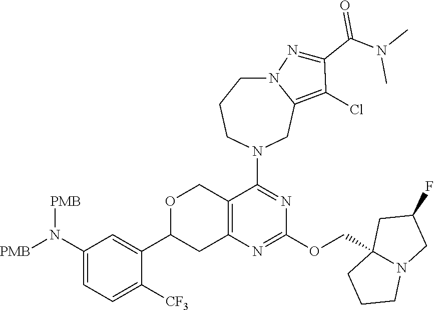

To a mixture of 5-(7-(5-(bis(4-methoxybenzyl)amino)-2-(trifluoromethyl) phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (85 mg, 0.10 mmol, 1.0 eq), ((2R,7aS)-2-fluorotetrahydro-1H-pyrrolizin-7a(5H)-yl)methanol (32 mg, 0.19 mmol, 2.0 eq) in tetrahydrofuran (2 mL) was added sodium t-butanolate (29 mg, 0.29 mmol, 3.0 eq). The reaction mixture was stirred at 25° C. for 0.5 h. LCMS showed the reaction was completed. The reaction mixture was quenched by saturated aqueous ammonium chloride (20 mL) and extracted with ethyl acetate (10 mL×3). The combined organic layers were washed with brine (20 mL), dried over anhydrous sodium sulfate, filtered and then concentrated. The residue was purified by Prep-TLC (Dichloromethane/Methanol=9/1) to afford the desired product (46 mg, 0.05 mmol, 40% yield) as a light yellow solid. LCMS (ESI, m/z): 933.5[M+H]⁺.

### Step 12: 5-(7-(5-amino-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

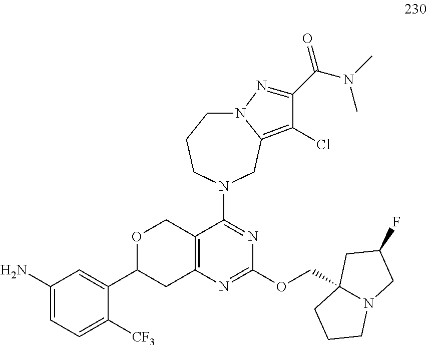 [SMILES: CN(C)C(=O)c1nn2c(c1Cl)CN(c1nc(OC[C@@]34CCCN3C[C@H](F)C4)nc3c1COC(c1cc(N)ccc1C(F)(F)F)C3)CCC2]

To a mixture of 5-(7-(5-(bis(4-methoxybenzyl)amino)-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (46 mg, 0.05 mmol, 1.0 eq) in dichloromethane (2 mL) was added trifluoroacetic acid (3.07 g, 26.92 mmol, 2 mL, 546 eq). The mixture was stirred at 40° C. for 2 h. LCMS showed the reaction was completed. The reaction mixture was concentrated under reduced pressure to give a residue which was purified by Prep-HPLC (column: Welch Xtimate C18 150*25 mm*5 um; mobile phase: [water(FA)-ACN]; gradient:10%-40% B over 40 min) to afford the title compound (4.61 mg, 0.006 mmol, 12% yield, 91% purity) as a white solid. LCMS (ESI, m/z): 693.3 [M+H]⁺. ¹H NMR (400 MHz, DMSO-d₆) δ: 7.33 (d, J=8.8 Hz, 1H), 6.83 (s, 1H), 6.50 (s, 1H), 5.88 (s, 2H), 5.36-5.13 (m, 1H), 5.08-4.98 (m, 1H), 4.91 (br dd, J=2.8, 10.8 Hz, 1H), 4.75-4.64 (m, 2H), 4.45-4.33 (m, 2H), 3.94-3.78 (m, 3H), 3.75-3.66 (m, 1H), 3.11-3.02 (m, 3H), 2.99 (s, 3H), 2.96 (s, 3H), 2.88-2.65 (m, 4H), 2.26-2.17 (m, 1H), 2.08-1.91 (m, 4H), 1.82-1.66 (m, 3H).

### Example 230a and 230b

5-((S)-7-(5-amino-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5, 6,7,8-tetrahydro-4H-pyrazolo[1,5-a][1,4]diazepine-2-carboxamide (Example 230a) and 5-((R)-7-(5-amino-2-(trifluoromethyl)phenyl)-2-(((2R,7aS)-2-fluorotetrahydro-1H-pyrrolizin-7a( 5H)-yl)methoxy)-7,8-dihydro-5H-pyrano[4,3-d]pyrimidin-4-yl)-3-chloro-N,N-dimethyl-5,6,7, 8-tetrahydro-4H-pyrazolo[1,5-a][1,4]diazepine-2-carboxamide

### Example 230b

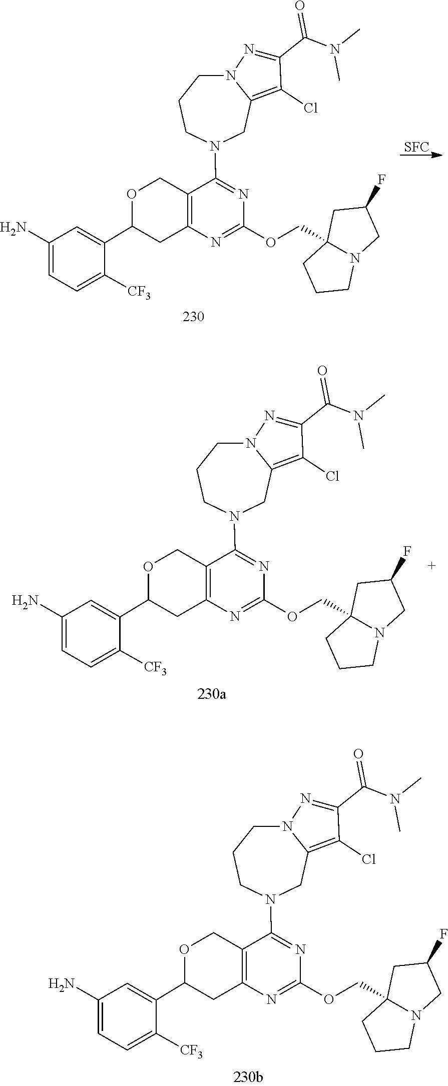

(5-(7-(5-Amino-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide) (110 mg, 0.15 mmol, 1.0 eq) was separated by SFC (column: DAICEL CHIRALCEL OD(250 mm*30 mm, 10 um); mobile phase: [CO₂-MeOH (0.1% NH₃H₂O)]; B %:40%, isocratic elution mode).

The first eluent was identified as 230a, 5-((S)-7-(5-amino-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (17.24 mg, 0.02 mmol, 15% yield, 95% purity, t_{R}=2.390 min) as a white solid. LCMS (ESI, m/z): 693.3 [M+H]⁺, ¹H NMR (400 MHz, DMSO-d₆) δ: 7.33 (d, J=8.8 Hz, 1H), 6.87 (d, J=2.0 Hz, 1H), 6.58 (dd, J=2.0, 8.8 Hz, 1H), 5.89 (s, 2H), 5.32 (br s, 1H), 5.18 (br s, 1H), 5.03 (br d, J=13.6 Hz, 1H), 4.94-4.79 (m, 2H), 4.75-4.60 (m, 2H), 4.47-4.34 (m, 2H), 3.95 (d, J=10.4 Hz, 1H), 3.91-3.78 (m, 2H), 3.76-3.63 (m, 1H), 3.06 (br d, J=7.6 Hz, 2H), 2.98 (s, 3H), 2.96 (s, 3H), 2.77 (br s, 2H), 2.74-2.67 (m, 1H), 2.25-2.15 (m, 1H), 2.09-2.01 (m, 2H), 1.98-1.85 (m, 2H), 1.83-1.68 (m, 3H).

The second eluent was identified as 230b, 5-((R)-7-(5-amino-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (26.37 mg, 0.03 mmol, 28% yield, 95% purity, t_{R}=2.534 min) as a white solid. LCMS (ESI, m/z): 693.3 [M+H]⁺, ¹H NMR (400 MHz, DMSO-d₆) δ: 7.33 (d, J=8.8 Hz, 1H), 6.89-6.84 (m, 1H), 6.57 (dd, J=2.0, 8.8 Hz, 1H), 5.89 (s, 2H), 5.28 (s, 1H), 5.21-5.14 (m, 1H), 5.07-4.99 (m, 1H), 4.94-4.79 (m, 2H), 4.75-4.64 (m, 2H), 4.47-4.32 (m, 2H), 3.93-3.84 (m, 3H), 3.03 (br s, 2H), 2.99 (s, 3H), 2.96 (s, 3H), 2.83-2.76 (m, 2H), 2.75-2.65 (m, 2H), 2.24-2.16 (m, 1H), 2.10-2.01 (m, 2H), 1.98-1.89 (m, 2H), 1.84-1.71 (m, 3H).

### 5-(7-(3-(bis(4-Methoxybenzyl)amino)-2-fluoro-6-(trifluoromethyl)phenyl)-2-((2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (3) (Example 231) (Mix of 4 diasteromers)

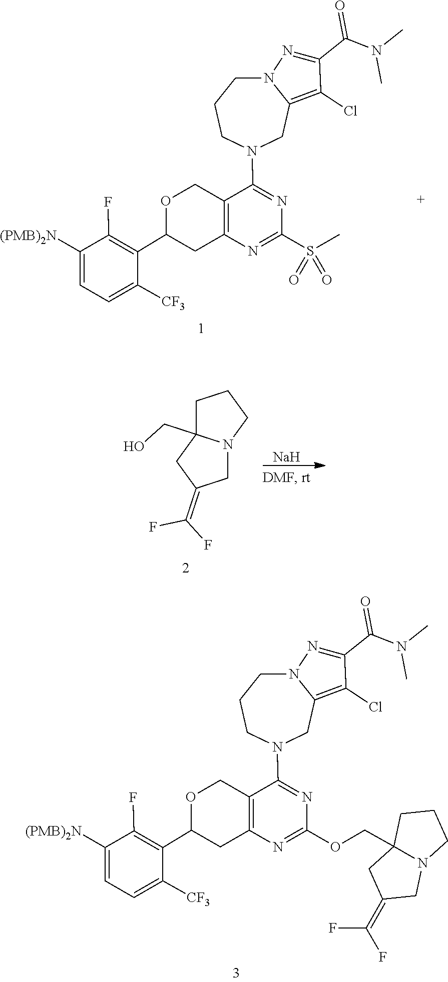

A flame dried 1 dram vial was charged with (2-(difluoromethylene)tetrahydro-1H-pyrrolizin-7a(5H)-yl)methanol (2, 43.5 mg 0.23 mmol) and dry dimethylformamide (5 mL). To this was added sodium hydride (100 mg, 21.8 mmol). The mixture was stirred at room temperature for 30 minutes. To this stirred solution was added 5-(7-(3-(bis(4-methoxybenzyl)amino)-2-fluoro-6-(trifluoromethyl)phenyl)-2-(methylsulfonyl)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (1, 100 mg, 0.115 mmol) in dimethylformamide (1 mL). The mixture was stirred at room temperature for 2 h. The mixture poured into saturated aqueous ammonium chloride. The aqueous phase was extracted with ethyl acetate three times. The combined organic layers were washed with brine, dried over anhydrous sodium sulfate, filtered, and concentrated under reduced pressure. Purification by flash column chromatography (C18 column, 10-100% acetonitrile in 0.1% aqueous ammonium bicarbonate) gave 5-(7-(3-(bis(4-methoxybenzyl)amino)-2-fluoro-6-(trifluoromethyl)phenyl)-2-((2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (3) as white solid. Yield: 97 mg, 87%; MS 981.2 [M+H]⁺.

### 5-(7-(3-Amino-2-fluoro-6-(trifluoromethyl)phenyl)-2-((2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 231, mixture of 4 diasteromers)

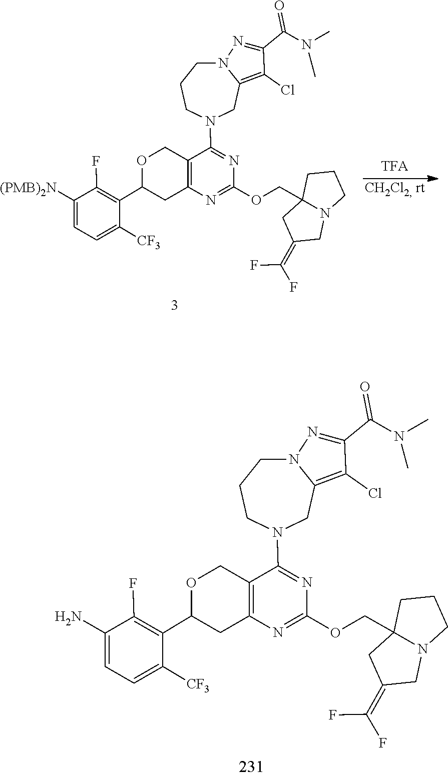

A 1 dram vial was charged with 5-(7-(3-(bis(4-methoxybenzyl)amino)-2-fluoro-6-(trifluoromethyl)phenyl)-2-((2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (3, 92.0 mg, 0.937 mmol) and dichloromethane (2 mL). To this stirred solution was added trifluoroacetic acid (0.44 mL) at 0° C. and stirred for 30 min. Then temperature was increased to room temperature and the reaction was stirred for further 3 hours. The mixture was concentrated to dryness under reduced pressure. Purification by flash column chromatography (C18 column, 5-100% acetonitrile in 0.1% aqueous ammonium bicarbonate) gave 5-(7-(3-amino-2-fluoro-6-(trifluoromethyl)phenyl)-2-((2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 231) as white solid. Yield; 22 mg, 32%; MS 741.18 [M+H]+; ¹H NMR (DMSO-d₆, 400 MHz): δ 7.24 (1H, d, J=8.7 Hz), 6.81 (1H, t, J=8.5 Hz), 5.94 (2H, s), 5.01-4.93 (2H, m), 4.85 (1H, m), 4.65-4.60 (2H, m), 4.42-4.31 (2H, m), 3.99-3.84 (3H, m), 3.68-3.55 (2H, m), 3.25 (1H, s), 3.10 (1H, m), 2.95 (3H, s), 2.93 (3H, s), 2.82-2.70 (1H, m), 2.52 (2H, brs), 2.39-2.27 (1H, m), 2.26-2.12 (1H, m), 2.09-1.95 (1H, m), 1.91-1.64 (4H, m). Note: One proton is unaccounted for. ¹⁹F NMR (DMSO-d₆, 376 MHz): δ−132.4, −91.2, −54.2.

### Examples 231a and 231b

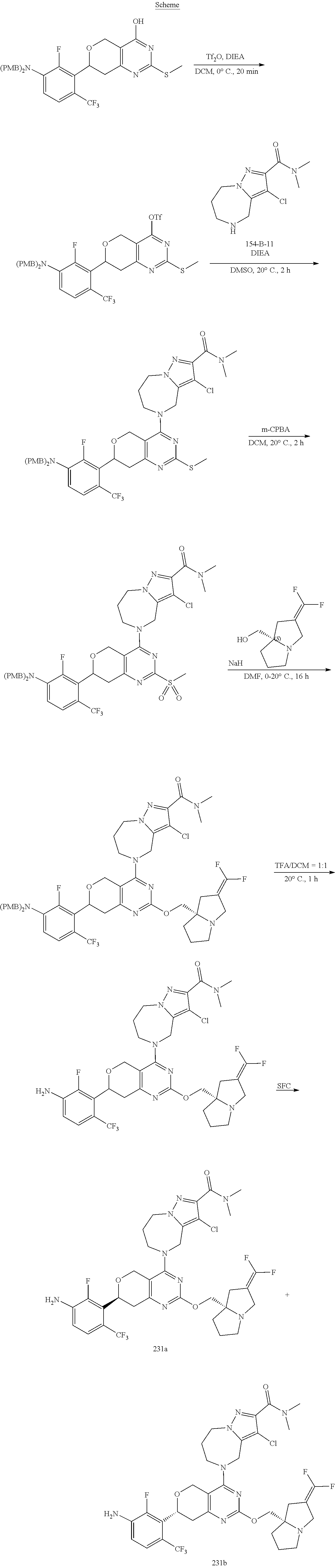

### Step 1: Synthesis of 7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-yl trifluoromethanesulfonate

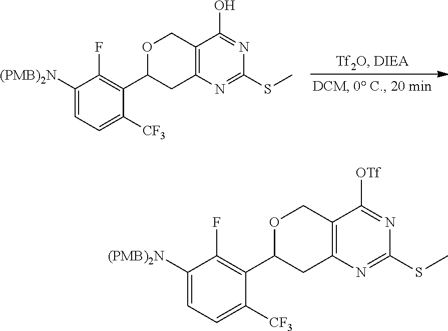

To a mixture of 7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-ol (500 mg, 0.812 mmol, 1 eq.) and DIEA (524 mg, 4.06 mmol, 5 eq.) in DCM (15 mL) was added dropwise of Tf₂O (460 mg, 1.62 mmol, 2 eq.) at 0° C. The mixture was stirred at 0° C. for 20 min. Then the mixture was diluted with NH₄Cl solution and extracted with DCM (50 mL) and water (50 mL). The organic layer was washed with water followed by brine. The organic phase was dried over anhydrous Na₂SO₄. The organic phase was concentrated under reduced pressure to give residue. The residue was purified by chromatography silica gel (PE:EA=5:1) to afford 7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-yl trifluoromethanesulfonate (625 g, 92.6% yield) as white solid. ESI-MS m/z=748.0 [M+H]⁺. Calculated MW: 747.13.

### Step 2: Synthesis of 5-(7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

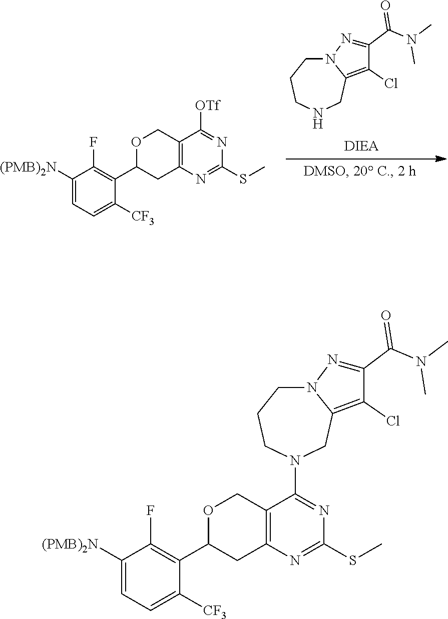

To a mixture of 7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-yl trifluoromethanesulfonate (400 mg, 0.535 mmol, 1 eq.) and 3-chloro-N,N-dimethyl-5,6,7,8-tetrahydro-4H-pyrazolo[1,5-a][1,4]diazepine-2-carboxamide (324 mg, 1.34 mmol, 2.5 eq.) in DMSO (15 mL) was added DIEA (690 mg, 5.35 mmol, 10 eq.) at 20° C. The mixture was stirred at 20° C. for 2 h. Then the mixture was extracted with EA (100 mL) and water (50 mL*3). The organic phase was washed with water followed by brine. The organic phase was dried over anhydrous Na₂SO₄. The organic phase was concentrated under reduced pressure to give residue. The residue was purified by chromatography silica gel (EA) to afford 5-(7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (425 mg, 75.6% yield) as white solid. ESI-MS m/z=840.2[M+H]⁺. Calculated MW: 839.26

### Step 3: Synthesis of 5-(7-(3-(bis(4-methoxybenzyl)amino)-2-fluoro-6-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

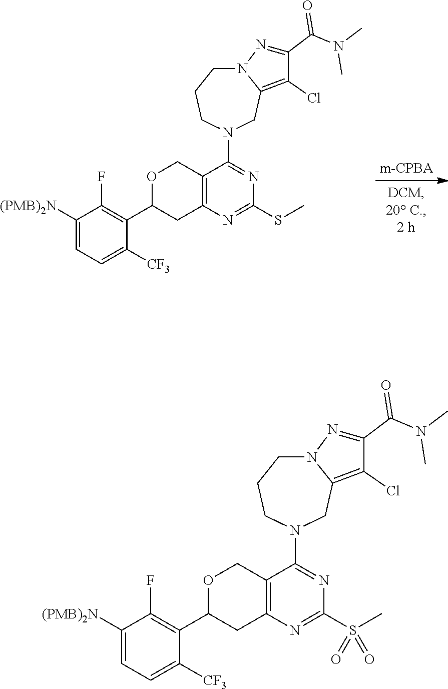

To a solution of 5-(7-(3-(bis(4-methoxybenzyl)amino)-2-fluoro-6-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (400 mg, 0.47 mmol, 1 eq.) in DCM (15 mL) was added m-CPBA (247 mg, 1.43 mmol, 3.0 eq.) at 20° C. The mixture was stirred at 20° C. for 2 h. Then the mixture was extracted with DCM (30 mL) and water (30 mL). The organic phase was washed with water followed by brine. The organic phase was dried over anhydrous Na₂SO₄. The organic phase was concentrated under reduced pressure to give residue. The residue was purified by Pre-TLC (EA ) to afford 5-(7-(3-(bis(4-methoxybenzyl)amino)-2-fluoro-6-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (305 mg, 62.4% yield) as solid. ESI-MS m/z=872.2 [M+H]⁺. Calculated MW: 871.25

### Step 4: Synthesis of 5-(7-(3-(bis(4-methoxybenzyl)amino)-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

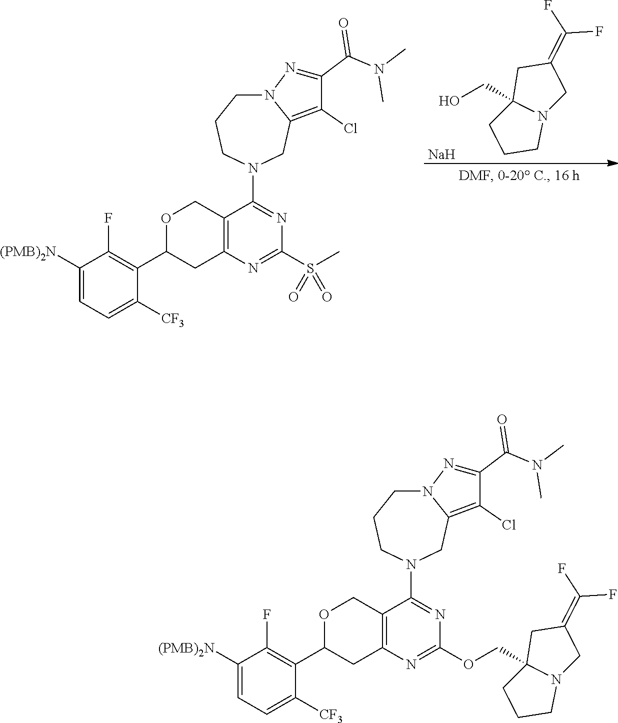

To the solution of (S)-(2-(difluoromethylene)tetrahydro-1H-pyrrolizin-7a(5H)-yl)methanol (91 mg, 0.482 mmol, 3 eq.) in 10 mL DMF was added 60% NaH (32.1 mg, 0.803 mmol, 5 eq.) at 0° C. and stirred for 20 min. Then 5-(7-(3-(bis(4-methoxybenzyl)amino)-2-fluoro-6-(trifluoromethyl)phenyl)-2-(methylsulfonyl)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (140 mg, 0.16 mmol, 1 eq.) was added into the mixture and stirred for 16 h at 20° C. The solution was diluted with NH₄Cl solution (5 mL) and extracted with EA (50 mL×3). The combined organic layers were washed with brine, dried over anhydrous sodium sulfate, filtered, and concentrated in vacuo. The residue was purified by Pre-TLC (DCM:MeOH=15:1) to afford 5-(7-(3-(bis(4-methoxybenzyl)amino)-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (142 mg, 72.1% yield) as solid. ESI-MS m/z=981.2 [M+H]⁺. Calculated MW: 980.36

### Step 5: Synthesis of 5-(7-(3-amino-2-fluoro-6-(trifluoromethyl)phenyl)-2-(((S)-2-(difluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

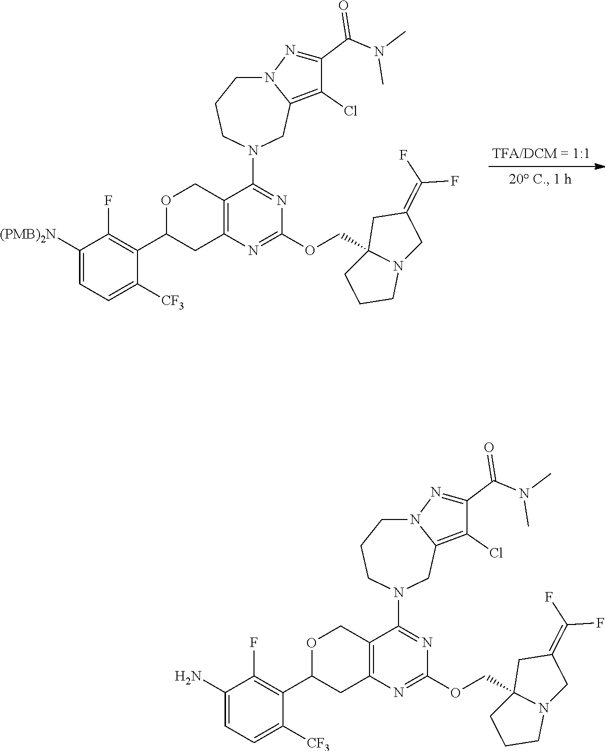

To the solution of 5-(7-(3-(bis(4-methoxybenzyl)amino)-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (130 mg, 0.132 mmol, 1 eq.) in 5 mL DCM was added TFA (5 mL) at 20° C. and stirred for 1 h. Upon completion, the solution was evaporated under reduced pressure. The mixture was purified by Prep-HPLC (ACN:H₂O=25%-55%, mobile phase: 0.1% TFA) to afford of 5-(7-(3-amino-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (35 mg, 32.1% yield) as solid. ESI-MS m/z=741.2[M+H]⁺. Calculated MW: 740.24.

### Step 6: Synthesis of 5-((S)-7-(3-amino-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((R)-7-(3-amino-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

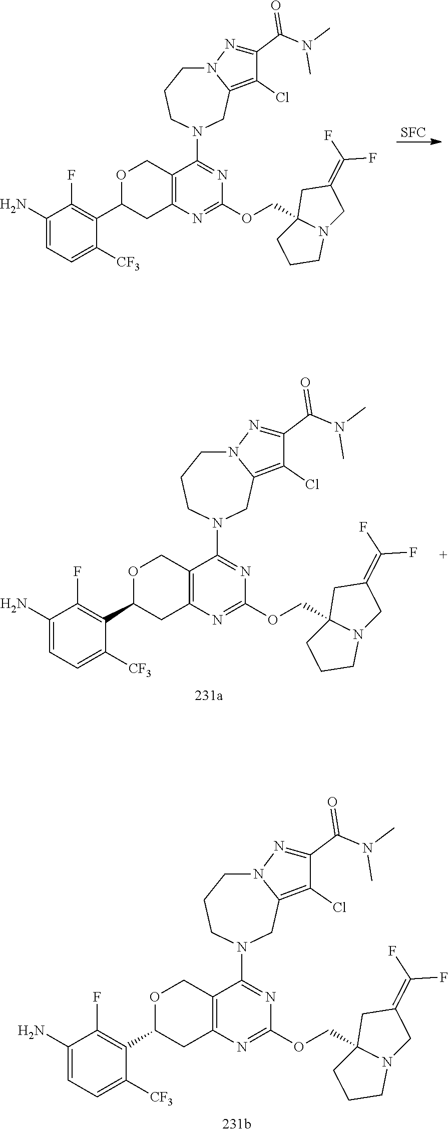

The product 5-(7-(3-amino-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (80 mg, TFA salt, 0.108 mmol) was sent to chiral separation by SFC (Mobile Phase:CO₂-MeOH) to afford 5-((S)-7-(3-amino-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 231a) (12.9 mg, 98.47% purity) as white solid and 5-((R)-7-(3-amino-2-fluoro-6-(trifluoromethyl)phenyl)-2-(((S)-2-(difluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 231b) (14.2 mg, 98.04% purity) as white solid.

### Example 231a

ESI-MS m/z=741.2 [M+H]⁺. Calculated MW: 740.24. ¹H NMR (400 MHz, DMSO) δ 7.25 (d, J=8.6 Hz, 1H), 6.84 (t, J=8.5 Hz, 1H), 5.14 (dd, J=11.2, 4.0 Hz, 1H), 5.04 (d, J=13.7 Hz, 1H), 4.81 (d, J=1.9 Hz, 2H), 4.74 (d, J=13.7 Hz, 1H), 4.52-4.44 (m, 1H), 4.35 (dd, J=13.6, 9.5 Hz, 1H), 4.11 (dd, J=24.0, 10.7 Hz, 2H), 4.00 (dt, J=13.8, 3.9 Hz, 1H), 3.79-3.71 (m, 2H), 3.39 (d, J=14.3 Hz, 1H), 3.30-3.24 (m, 1H), 3.14-3.05 (m, 7H), 2.84 (dd, J=17.9, 4.2 Hz, 1H), 2.67 (dd, J=15.5, 4.6 Hz, 2H), 2.44 (d, J=15.9 Hz, 1H), 2.34 (dd, J=13.2, 6.4 Hz, 1H), 2.20-2.13 (m, 1H), 2.04 (dd, J=9.2, 5.9 Hz, 1H), 1.98-1.91 (m, 1H), 1.89-1.82 (m, 2H).

### Example 231b

ESI-MS m/z=741.2 [M+H]⁺. Calculated MW: 740.24

¹H NMR (400 MHz, DMSO) δ 7.25 (d, J=8.6 Hz, 1H), 6.85 (t, J=8.5 Hz, 1H), 5.14 (dd, J=11.2, 4.0 Hz, 1H), 5.04 (d, J=13.7 Hz, 1H), 4.81 (d, J=5.0 Hz, 2H), 4.74 (t, J=8.4 Hz, 1H), 4.52-4.44 (m, 1H), 4.36 (dd, J=13.5, 9.5 Hz, 1H), 4.21 (d, J=10.7 Hz, 1H), 3.99 (d, J=10.8 Hz, 2H), 3.79-3.71 (m, 2H), 3.39 (d, J=14.2 Hz, 1H), 3.30-3.24 (m, 1H), 3.08 (t, J=4.9 Hz, 7H), 2.84 (dd, J=17.8, 4.2 Hz, 1H), 2.74-2.62 (m, 2H), 2.46 (d, J=15.8 Hz, 1H), 2.35 (dd, J=19.6, 9.8 Hz, 1H), 2.16 (dd, J=11.7, 6.9 Hz, 1H), 2.06 (dd, J=9.5, 5.8 Hz, 1H), 1.94 (ddd, J=8.6, 6.6, 3.1 Hz, 1H), 1.90-1.83 (m, 2H).

### 5-(7-(3-amino-2-fluoro-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N-cyclopropyl-N-methyl-5,6,7,8-tetrahydro-4H-pyrazolo[1,5-a][1,4]diazepine-2-carboxamide (Example 232)

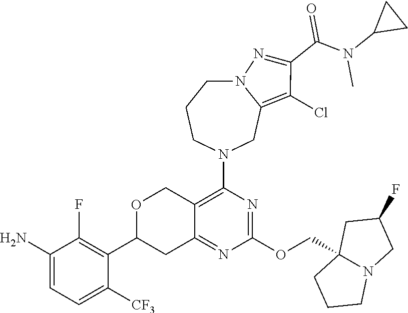 [SMILES: CN(C(=O)c1nn2c(c1Cl)CN(c1nc(OC[C@@]34CCCN3C[C@H](F)C4)nc3c1COC(c1c(C(F)(F)F)ccc(N)c1F)C3)CCC2)C1CC1]

¹H NMR (400 MHz, MeOH-d₄) δ 8.43 (br s, 1H), 7.26 (d, J=8.5 Hz, 1H), 6.86 (t, J=8.5 Hz, 1H), 5.68-5.41 (m, 1H), 5.16 (dd, J=11.4, 4.3 Hz, 1H), 5.02 (d, J=13.9 Hz, 1H), 4.98-4.90 (m, 1H), 4.77 (dd, J=13.8, 3.2 Hz, 1H), 4.66 (d, J=16.4 Hz, 1H), 4.53-4.37 (m, 2H), 4.32-4.14 (m, 2H), 4.05-3.66 (m, 5H), 3.44-3.33 (m, 1H), 3.30-3.22 (m, 1H), 3.17-3.01 (m, 3H), 3.00-2.92 (m, 1H), 2.87 (dd, J=18.0, 4.4 Hz, 1H), 2.71-2.46 (m, 2H), 2.45-2.32 (m, 2H), 2.31-2.20 (m, 2H), 2.19-2.05 (m, 2H), 1.00-0.69 (m, 1H), 0.68-0.40 (m, 3H); LRMS-ESI (m/z) [M+H]⁺ calculated for C₃₄H₃₉ClF₅N₈O₃ 737.28, found 737.3.

### 5-(7-(3-amino-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 233)

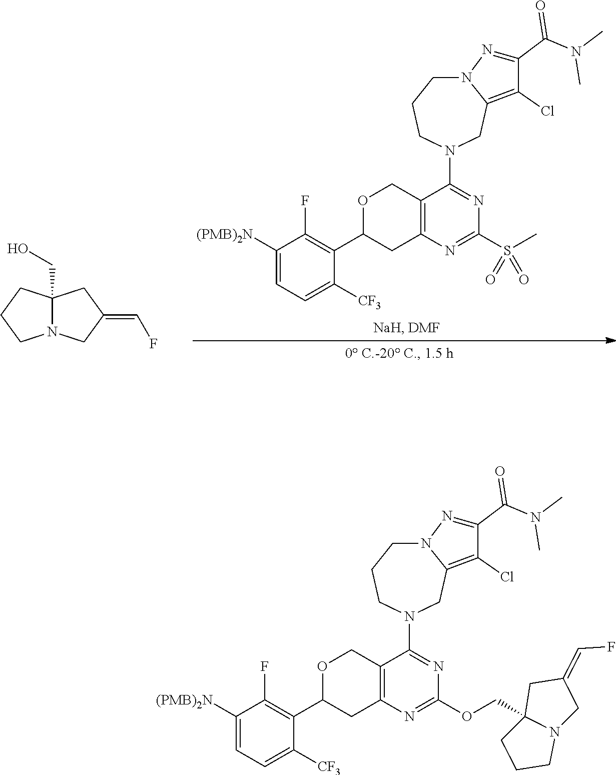

To the solution of (S,Z)-(2-(fluoromethylene)tetrahydro-1H-pyrrolizin-7a(5H)-yl)methanol (59 mg, 0.34 mmol) in 8 mL DMF was added 60% NaH (23 mg, 0.57 mmol) at 0° C. and stirred for 20 min. Then 5-(7-(3-(bis(4-methoxybenzyl)amino)-2-fluoro-6-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (100 mg, 0.115 mmol) was added into the mixture and stirred for 1.5 h at 20° C. The solution was diluted with NH₄Cl solution and extracted with ethyl acetate three times. The combined organic layers were washed with brine, dried over anhydrous sodium sulfate, filtered and concentrated in vacuo. The residue was purified by preparative thin layer chromatography (ethyl acetate:methanol=20:1) to afford 5-(7-(3-(bis(4-methoxybenzyl)amino)-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (90 mg, 73% yield) as solid. ESI-MS m/z=963.2 [M+H]⁺. Calculated MW: 962.37

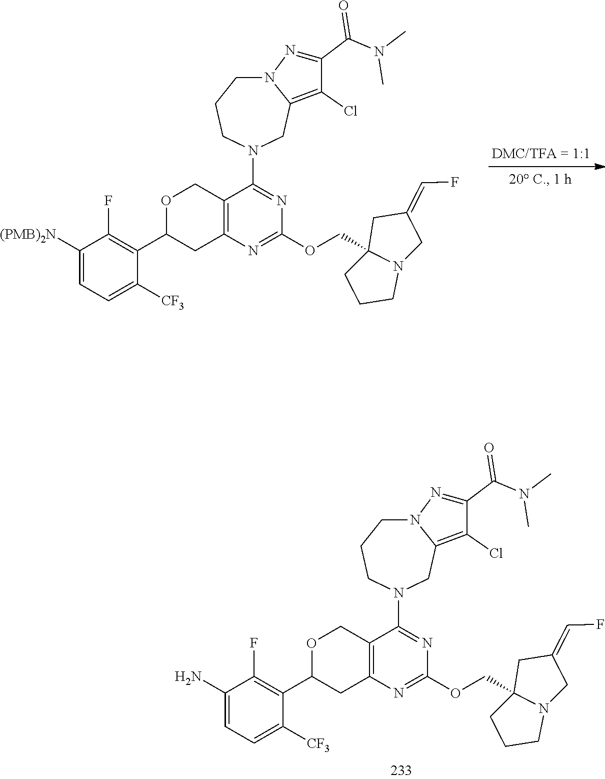

To the solution of 5-(7-(3-(bis(4-methoxybenzyl)amino)-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (85 mg, 0.0882 mmol) in 5 mL dichloromethane was added trifluoriacetic acid (5 mL) at 20′C and stirred for 1 h. Upon completion, the solution was evaporated under reduced pressure. The residue was purified by preparative HPLC (acetonitrile:water=25%-55%, mobile phase: 0.1% trifluoroacetic acid) to afford 5-(7-(3-amino-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (30 mg, 45.6% yield) as solid. ESI-MS m/z=723.2[M+H]⁺. Calculated MW: 722.25. ¹H NMR (400 MHz, CD3OD) δ 7.29 (d, J=8.6 Hz, 1H), 7.04-6.79 (m, 2H), 5.19 (dd, J=11.1, 4.1 Hz, 1H), 5.04 (d, J=10.3 Hz, 2H), 4.83-4.68 (m, 2H), 4.65-4.36 (m, 4H), 4.27 (d, J=9.9 Hz, 1H), 4.15 (d, J=14.5 Hz, 1H), 4.02 (s, 1H), 3.93-3.76 (m, 2H), 3.37 (s, 1H), 3.28 (d, J=6.9 Hz, 1H), 3.18-3.10 (m, 6H), 3.07-2.90 (m, 2H), 2.80 (t, J=15.6 Hz, 1H), 2.49-2.33 (m, 2H), 2.31-2.12 (m, 4H).

### 5-((S)-7-(5-amino-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 234)

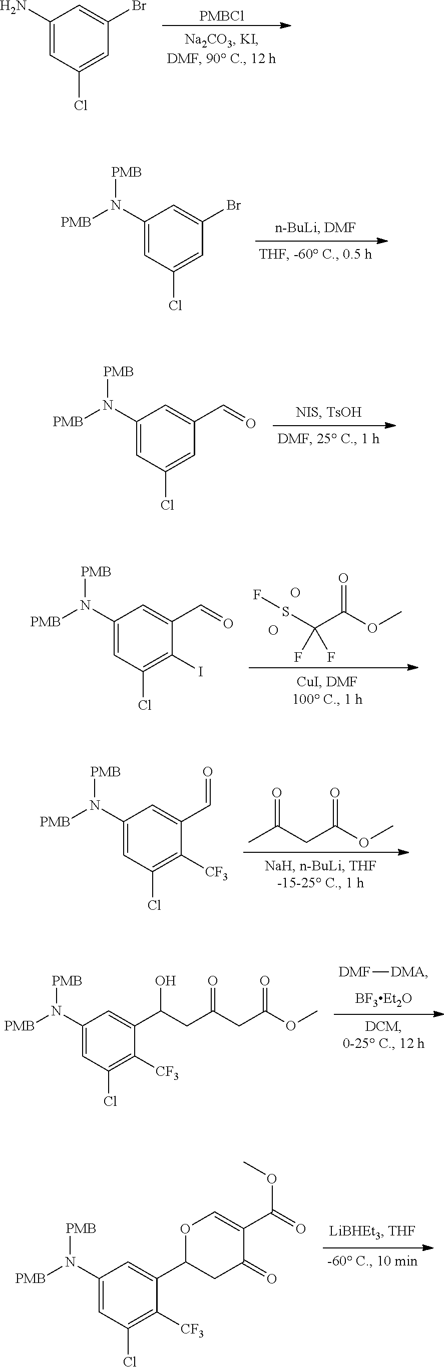

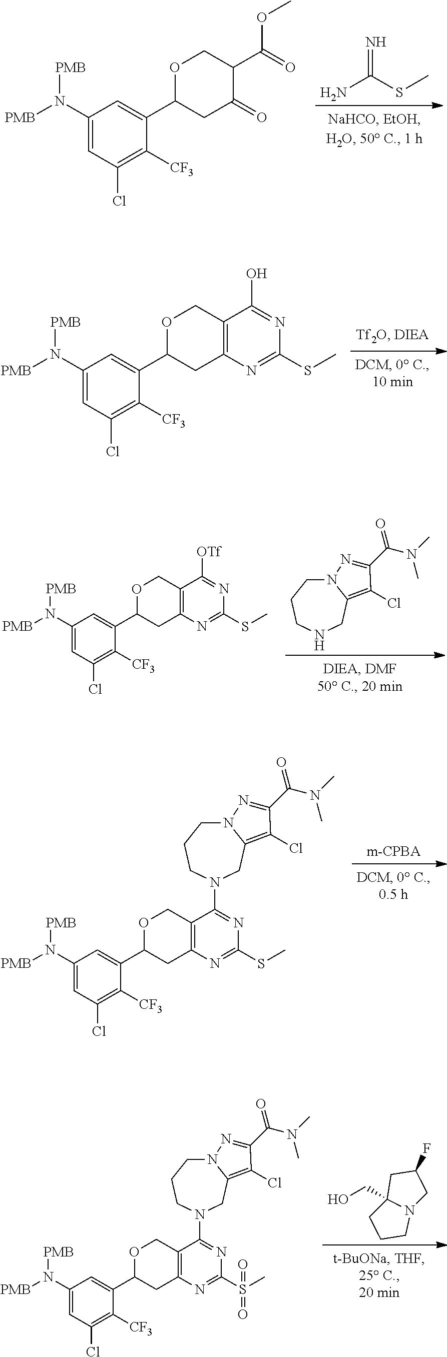

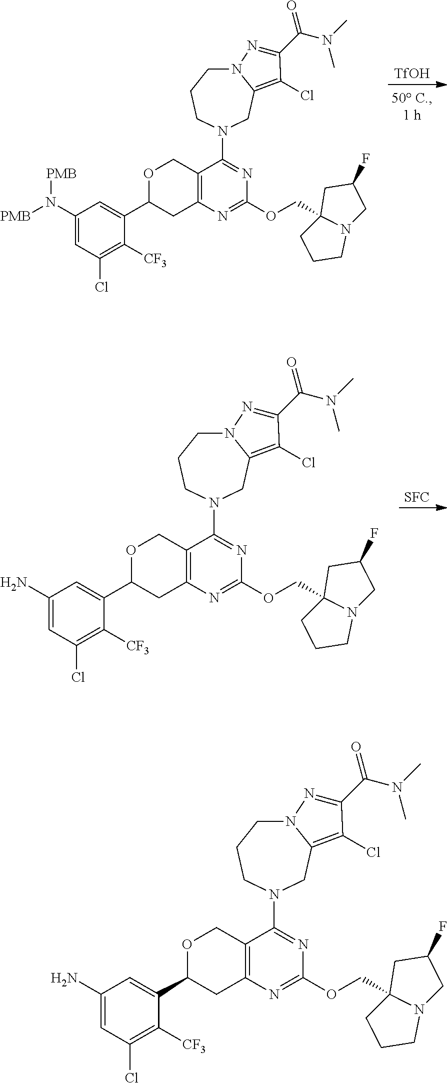

### Step 1: 3-bromo-5-chloro-N,N-bis(4-methoxybenzyl)aniline

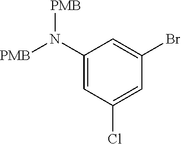

To a solution of 3-bromo-5-chloroaniline (25 g, 121.08 mmol, 1.0 eq) in N,N-dimethylformamide (200 mL) was added p-methoxybenzyl chloride (43.61 g, 278.49 mmol, 37.8 mL, 2.3 eq), potassium iodide (12.06 g, 72.65 mmol, 0.6 eq) and sodium carbonate (32.08 g, 302.71 mmol, 2.5 eq), then the mixture was stirred at 90° C. for 12 h. The reaction mixture was diluted with water (800 mL) and extracted with ethyl acetate (300 mL×3). The combined organic phase was washed with brine (500 mL×2), dried over anhydrous sodium sulfate, filtered and concentrated in vacuum to give a residue. The residue was purified by silica gel chromatography (petroleum ether/ethyl acetate=50/1 to 10/1) to afford 3-bromo-5-chloro-N,N-bis(4-methoxybenzyl) aniline (21 g, 42.30 mmol, 38% yield, 90% purity) as a yellow solid. ¹H NMR (400 MHz, DMSO-d₆) δ: 7.34-7.20 (m, 5H), 7.05 (s, 1H), 6.88-6.71 (m, 5H), 4.55 (s, 4H), 3.70 (s, 6H).

### Step 2: 3-(bis(4-methoxybenzyl)amino)-5-chlorobenzaldehyde

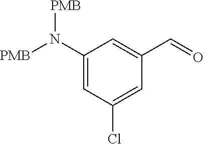

A solution of 3-bromo-5-chloro-N,N-bis(4-methoxybenzyl) aniline (21 g, 42.30 mmol, 1.0 eq) in tetrahydrofuran (200 mL) was degassed and purged with nitrogen three times, n-butyllithium (2.5 M, 27.5 mL, 1.5 eq) was added at −60° C. The reaction mixture was stirred at −60° C. for 0.5 h under nitrogen. Then N,N-dimethylformamide (10.2 g, 129.2 mmol, 10.8 mL, 3.0 eq) was added at −60° C., and the mixture was stirred at −60° C. for 0.5 h. The reaction mixture was quenched by adding saturated aqueous ammonium chloride (400 mL) and extracted with ethyl acetate (60 mL×3). The combined organiclayers were washed with brine (200 mL×2), dried over anhydrous sodium sulfate, filtered and concentrated in vacuum to give a residue. The residue was purified by flash silica gel chromatography (petroleum ether/ethyl acetate=10/1) to afford 3-(bis(4-methoxybenzyl)amino)-5-chlorobenzaldehyde (12 g, 30.31 mmol, 24% yield) as a yellow solid. ¹H NMR (400 MHz, DMSO-d₆) δ: 9.86-9.83 (m, 1H), 7.25-7.18 (m, 5H), 7.13 (s, 1H), 6.99-6.91 (m, 5H), 4.76 (s, 4H), 3.76 (s, 6H).

### Step 3: 5-(bis(4-methoxybenzyl)amino)-3-chloro-2-iodobenzaldehyde

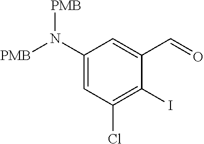

To a solution of 3-(bis(4-methoxybenzyl)amino)-5-chlorobenzaldehyde (12 g, 30.31 mmol, 1.0 eq) in N,N-dimethylformamide (150 mL) was added toluene-4-sulfonic acid (5.22 g, 30.31 mmol, 1.0 eq) and N-iodo-succinimide (8.18 g, 36.37 mmol, 1.2 eq). The mixture was stirred at 25° C. for 1 h. The reaction mixture was quenched by the addition of saturated aqueous sodium bicarbonate (200 mL) and extracted with ethyl acetate (50 mL×3). The combined organic layers were washed with brine (100 mL×2), dried over anhydrous sodium sulfate, filtered and concentrated in vacuum to give a residue. The residue was purified by flash silica gel chromatography (petroleum ether/ethyl acetate=10/1 to 5/1) to afford 5-(bis(4-methoxybenzyl)amino)-3-chloro-2-iodobenzaldehyde (12.7 g, 24.34 mmol, 80% yield) as a yellow solid. ¹H NMR (400 MHz, DMSO-d₆) δ: 9.91 (s, 1H), 7.21 (br d, J=8.8 Hz, 5H), 7.06 (d, J=2.8 Hz, 1H), 6.94 (d, J=8.8 Hz, 4H), 4.73 (s, 4H), 3.76 (s, 6H).

### Step 4: 5-(bis(4-methoxybenzyl)amino)-3-chloro-2-(trifluoromethyl)benzaldehyde

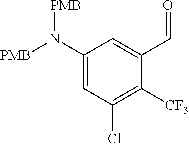

To a solution of 5-(bis(4-methoxybenzyl)amino)-3-chloro-2-iodobenzaldehyde (12.7 g, 24.34 mmol, 1.0 eq) in N,N-dimethylformamide (150 mL) was added methyl 2,2-difluoro-2-(fluorosulfonyl)acetate (18.70 g, 97.36 mmol, 12.4 mL, 4.0 eq) and cuprous iodide (13.91 g, 73.02 mmol, 3.0 eq), the mixture was stirred at 100° C. for 1 h. The reaction mixture was diluted with water (300 mL) and extracted with ethyl acetate (60 mL×3). The combined organic layers were was washed with brine (200 mL×2), dried over anhydrous sodium sulfate, filtered and concentrated in vacuum to give a residue. The residue was purified by column chromatography (petroleum ether/ethyl acetate=100/1 to 20/1) to afford 5-(bis(4-methoxybenzyl)amino)-3-chloro-2-(trifluoromethyl)benzaldehyde (8.8 g, 18.98 mmol, 78% yield) as a white solid. ¹H NMR (400 MHz, DMSO-d₆) δ: 10.24 (s, J=3.6 Hz, 1H), 7.24 (d, J=8.8 Hz, 4H), 7.10 (d, J=2.4 Hz, 1H), 7.05 (d, J=2.4 Hz, 1H), 6.98 (d, J=8.8 Hz, 4H), 4.82 (s, 4H), 3.79 (s, 6H).

### Step 5: methyl 5-(5-(bis(4-methoxybenzyl)amino)-3-chloro-2-(trifluoromethyl)phenyl)-5-hydroxy-3-oxopentanoate

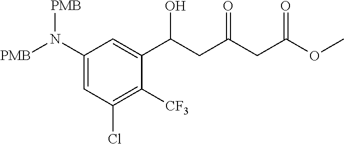

To a solution of methyl 3-oxobutanoate (5.5 g, 47.42 mmol, 5.1 mL, 2.5 eq) in tetrahydrofuran (100 mL) was added sodium hydride (1.89 g, 47.42 mmol, 60% purity, 2.5 eq) at 0° C., the mixture was stirred at 0° C. for 10 min. After cooling to −15° C., n-butyllithium (2.5 M, 19.0 mL, 2.5 eq) was added and the reaction mixture was stirred at −15° C. for 20 min. Then 5-(bis(4-methoxybenzyl)amino)-3-chloro-2-(trifluoromethyl)benzaldehyde (8.8 g, 18.98 mmol, 1.0 eq) was added, and the reaction mixture was stirred at −15° C. for 30 min. The reaction mixture was quenched by addition of saturated aqueous ammonium chloride (200 mL) and extracted with ethyl acetate (50 mL×2). The combined organic layers were was washed with brine (100 mL×2), dried over anhydrous sodium sulfate, filtered and concentrated in vacuum to give a residue. The residue was purified by flash silica gel chromatography (petroleum ether/ethyl acetate=10/1 to 3/1) to afford methyl 5-(5-(bis(4-methoxybenzyl)amino)-3-chloro-2-(trifluoromethyl)phenyl)-5-hydroxy-3-oxopentanoate (7.3 g, 12.59 mmol, 66% yield) as a yellow oil. ¹H NMR (400 MHz, DMSO-d₆) δ: 7.26-7.18 (m, 5H), 6.95 (d, J=8.8 Hz, 4H), 6.76 (s, 1H), 5.69-5.62 (m, 1H), 5.42-5.32 (m, 1H), 4.71 (br d, J=10.0 Hz, 2H), 3.77 (s, 6H), 3.65 (s, 3H), 2.69-2.61 (m, 1H), 2.61-2.57 (m, 2H), 2.03 (s, 3H).

### Step 6: methyl 2-(5-(bis(4-methoxybenzyl)amino)-3-chloro-2-(trifluoromethyl)phenyl)-4-oxo-3,4-dihydro-2H-pyran-5-carboxylate

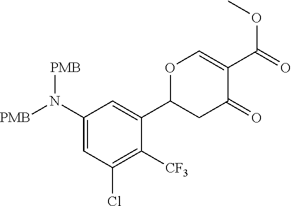

To a solution of methyl 5-(5-(bis(4-methoxybenzyl)amino)-3-chloro-2-(trifluoromethyl)phenyl)-5-hydroxy-3-oxopentanoate (7.3 g, 12.59 mmol, 1.0 eq) in dichloromethane (50 mL) was added N,N-dimethyl-formamide dimethyl acetal (3.00 g, 25.17 mmol, 3.3 mL, 2.0 eq). The mixture was stirred at 25° C. for 12 h. Then trifluoroborane diethyl ether (3.57 g, 25.17 mmol, 3.1 mL, 2.0 eq) was added at 0° C., and the reaction mixture was stirred at 0° C. for 10 min. LCMS showed the reaction was completed. The reaction mixture was quenched by adding saturated aqueous sodium bicarbonate (150 mL) and extracted with dichloromethane (40 mL×3). The combined organic layers were washed with brine (100 mL×2), dried over anhydrous sodium sulfate, filtered and concentrated in vacuum to give a crude product methyl 2-(5-(bis(4-methoxybenzyl)amino)-3-chloro-2-(trifluoromethyl)phenyl)-4-oxo-3,4-dihydro-2H-pyran-5-carboxylate (7.3 g, crude) as a yellow oil. LCMS (ESI, m/z): 590.2 [M+H]⁺.

### Step 7: methyl 6-(5-(bis(4-methoxybenzyl)amino)-3-chloro-2-(trifluoromethyl)phenyl)-4-oxotetrahydro-2H-pyran-3-carboxylate

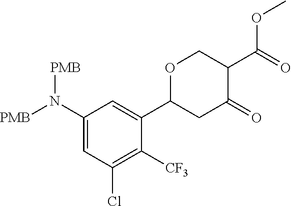

To a solution of methyl 2-(5-(bis(4-methoxybenzyl)amino)-3-chloro-2-(trifluoromethyl)phenyl)-4-oxo-3,4-dihydro-2H-pyran-5-carboxylate (7.3 g, 12.37 mmol, 1.0 eq) in tetrahydrofuran (70 mL) was added lithium triethylborohydride (1 M, 14.8 mL, 1.2 eq) slowly at −60° C. The mixture was stirred at −60° C. for 10 min. LCMS showed the reaction was completed. The reaction mixture was quenched by adding saturated aqueous ammonium chloride (200 mL) and extracted with ethyl acetate (40 mL×3). The combined organic layers were washed with brine (100 mL×2), dried over anhydrous sodium sulfate, filtered and concentrated in vacuum to give a residue. The residue was purified by flash silica gel chromatography (petroleum ether/ethyl acetate=10/1) to afford methyl 6-(5-(bis(4-methoxybenzyl)amino)-3-chloro-2-(trifluoromethyl)phenyl)-4-oxotetrahydro-2H-pyran-3-carboxylate (3.6 g, 6.08 mmol, 49% yield) as a light yellow solid. LCMS (ESI, m/z): 592.3 [M+H]⁺.

### Step 8: 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-ol

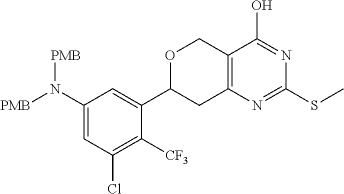

To a solution of methyl 6-(5-(bis(4-methoxybenzyl)amino)-3-chloro-2-(trifluoromethyl)phenyl)-4-oxotetrahydro-2H-pyran-3-carboxylate (3.6 g, 6.08 mmol, 1.0 eq), 2-methylisothiourea (5.48 g, 60.81 mmol, 10.0 eq) in ethanol (40 mL) and water (8 mL) was added sodium bicarbonate (10.22 g, 121.62 mmol, 4.7 mL, 20.0 eq). The mixture was stirred at 50° C. for 1 h. LCMS showed the reaction was completed. The reaction mixture was diluted with water (50 mL) and extracted with ethyl acetate (30 mL×3). The combined organic layers were washed with brine (60 mL×2), dried over anhydrous sodium sulfate, filtered and concentrated in vacuum to give a crude product 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-ol (3.8 g, crude) as a yellow oil. LCMS (ESI, m/z): 632.3 [M+H]⁺.

### Step 9: 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl trifluoromethanesulfonate

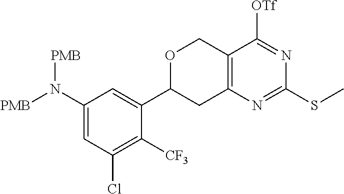

To a solution of 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-ol (3.8 g, 6.01 mmol, 1.0 eq) in dichloromethane (40 mL) was added N,N-diisopropylethylamine (3.88 g, 30.06 mmol, 5.2 mL, 5.0 eq). Then trifluoromethylsulfonic anhydride (3.39 g, 12.02 mmol, 2.0 mL, 2.0 eq) was added at 0° C., and the mixture was stirred at 0° C. for 10 min. LCMS showed the reaction was completed. The reaction mixture was diluted with saturated aqueous ammonium chloride (100 mL) and extracted with dichloromethane (20 mL×3). The combined organic layers were washed with brine (50 mL×2), dried over anhydrous sodium sulfate, filtered and concentrated in vacuum to give a residue. The residue was purified by flash silica gel chromatography (petroleum ether/ethyl acetate=1/0 to 10/1) to afford 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl trifluoromethanesulfonate (2 g, 2.62 mmol, 43% yield) as a yellow oil. LCMS (ESI, m/z): 764.2 [M+H]⁺.

### Step 10: 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

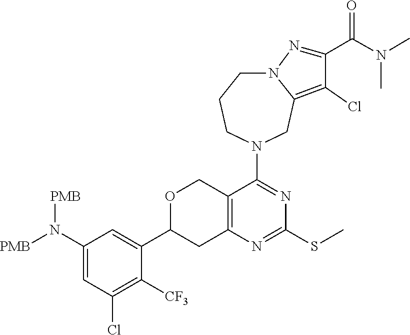

To a solution of 3-chloro-N,N-dimethyl-5,6,7,8-tetrahydro-4H-pyrazolo[1,5-a][1,4]diazepine-2-carboxamide (1.03 g, 2.88 mmol, 1.1 eq, trifluoroacetate) in N,N-diisopropylethylamine (1.01 g, 7.85 mmol, 1.4 mL, 3.0 eq) and N,N-dimethylformamide (10 mL) was added 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl trifluoromethanesulfonate (2 g, 2.62 mmol, 1.0 eq). The mixture was stirred at 50° C. for 20 min. LCMS showed the reaction was completed. The reaction mixture was diluted with water (40 mL) and extracted with ethyl acetate (20 mL×2). The combined organic layers were washed with brine (50 mL×2), dried over anhydrous sodium sulfate, filtered and concentrated in vacuum to give a residue. The residue was purified by flash silica gel chromatography (dichloromethane/methanol=10/1) to afford 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (1.5 g, 1.75 mmol, 66% yield) as a yellow solid. LCMS (ESI, m/z): 856.3 [M+H]⁺.

### Step 11: 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a]1[1,4]diazepine-2-carboxamide

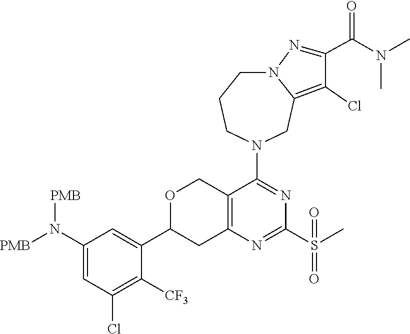

To a solution of 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (1.5 g, 1.75 mmol, 1.0 eq) in dichloromethane (10 mL) was added 3-chloro-benzenecarboperoxoic acid (781 mg, 3.85 mmol, 85% purity, 2.2 eq) at 0° C., and the reaction mixture was stirred at 0° C. for 0.5 h. LCMS showed the reaction was completed. The reaction mixture was quenched by adding saturated aqueous sodium sulfite (50 mL) and extracted with dichloromethane (15 mL×3). The combined organic layers were washed with saturated aqueous sodium bicarbonate (20 mL×2) and brine (30 mL×2), dried over anhydrous sodium sulfate, filtered and concentrated in vacuum to give a crude product 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (900 mg, crude) as a light yellow solid. LCMS (ESI, m/z): 872.3 [M+H]⁺.

### Step 12: 5-(7-(5-(bis(4-methoxybenzyl)amino)-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

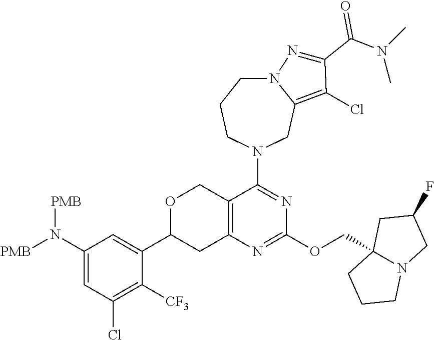

To a solution of 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (300 mg, 0.51 mmol, 1.0 eq) and ((2R,7aS)-2-fluorotetrahydro-1H-pyrrolizin-7a(5H)-yl)methanol (162 mg, 1.02 mmol, 2.0 eq) in tetrahydrofuran (8 mL) was added sodium t-butanolate (146 mg, 1.5 mmol, 3.0 eq) at 25° C. The mixture was stirred at 25° C. for 20 min. LCMS showed the reaction was completed. The reaction mixture was diluted with saturated aqueous ammonium chloride (50 mL) and extracted with ethyl acetate (15 mL×3), washed with brine (30 mL), dried over anhydrous sodium sulfate, filtered and then concentrated in vacuum to give a residue. The residue was purified by prep-TLC (dichloromethane/methanol=9/1) to afford 5-(7-(5-(bis(4-methoxybenzyl)amino)-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (150 mg, 0.18 mmol, 60% yield) as a yellow solid. LCMS (ESI, m/z): 967.3 [M+H]⁺.

### Step 13: 5-(7-(5-amino-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

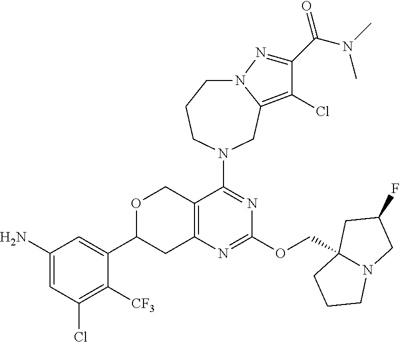 [SMILES: CN(C)C(=O)c1nn2c(c1Cl)CN(c1nc(OC[C@@]34CCCN3C[C@H](F)C4)nc3c1COC(c1cc(N)cc(Cl)c1C(F)(F)F)C3)CCC2]

A solution of 5-(7-(5-(bis(4-methoxybenzyl)amino)-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (150 mg, 0.18 mmol, 1.0 eq) in trifluoromethanesulfonic acid (10.72 g, 0.08 mmol, 7 mL, 199.5 eq) was stirred at 50° C. for 1 h. LCMS showed the reaction was completed. The reaction mixture was quenched by adding saturated aqueous sodium bicarbonate (20 mL), extracted with ethyl acetate (15 mL×2), dried over anhydrous sodium sulfate, filtered and then concentrated in vacuum to give a residue. The residue was purified by prep-TLC (dichloromethane/methanol=9/1) to afford 5-(7-(5-amino-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (55 mg, 0.05 mmol, 49% yield, 94% purity) as a light yellow solid. LCMS (ESI, m/z): 727.3 [M+H]⁺. ¹H NMR (400 MHz, DMSO-d₆) δ: 6.88 (s, 1H), 6.70 (d, J=2.0 Hz, 1H), 6.22 (s, 2H), 5.35-5.14 (m, 1H), 5.09-5.00 (m, 2H), 4.88-4.77 (m, 1H), 4.75-4.64 (m, 2H), 4.48-4.30 (m, 2H), 3.95 (d, J=10.8 Hz, 1H), 3.89-3.80 (m, 2H), 3.79-3.67 (m, 1H), 3.10-3.02 (m, 2H), 2.99 (s, 3H), 2.96 (s, 3H), 2.91-2.79 (m, 2H), 2.69-2.55 (m, 1H), 2.46-2.13 (m, 2H), 2.11-2.01 (m, 2H), 2.00-1.95 (m, 1H), 1.94-1.87 (m, 1H), 1.85-1.78 (m, 1H), 1.77-1.68 (m, 2H).

### Step 14: 5-((S)-7-(5-amino-3-chloro-2-(trifluoromethyl)phenyl)-2-(((2R,7aS)-2-fluorotetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 234)

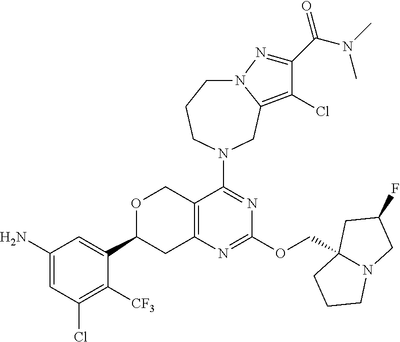 [SMILES: CN(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2]

The mixture of diastereomers 5-(7-(5-amino-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (55 mg, 0.05 mmol, 1.00 eq) was separated by SFC (column: DAICEL CHIRALPAK AD (250 mm*30 mm, 10 um); mobile phase: [CO₂-ACN/i-PrOH (0.1% NH₃H₂O)]; B %:45%, isocratic elution mode). The first eluent was identified as the desired diastereomer 5-((S)-7-(5-amino-3-chloro-2-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (23.56 mg, 0.032 mmol, 45% yield, 99% purity, t_{R}=1.575 min) as an off-white solid. LCMS (ESI, m/z): 727.3 [M+H]⁺. ¹H NMR (400 MHz, DMSO-d₆) δ: 6.91-6.84 (m, 1H), 6.70 (d, J=2.0 Hz, 1H), 6.26-6.15 (m, 2H), 5.33-5.13 (m, 1H), 5.10-4.98 (m, 2H), 4.88-4.76 (m, 1H), 4.75-4.64 (m, 2H), 4.47-4.27 (m, 2H), 3.91-3.79 (m, 3H), 3.78-3.66 (m, 1H), 3.08-3.01 (m, 2H), 2.99 (s, 3H), 2.96 (s, 3H), 2.89-2.79 (m, 2H), 2.68-2.57 (m, 1H), 2.36-2.11 (m, 2H), 2.10-2.00 (m, 2H), 1.98-1.89 (m, 2H), 1.85-1.77 (m, 1H), 1.75-1.62 (m, 2H).

### 5-((S)-7-(5-amino-3-chloro-2-(trifluoromethyl)phenyl)-2-(((S,Z)-2-(fluoromethylene)tetr ahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxa mide (Example 235)

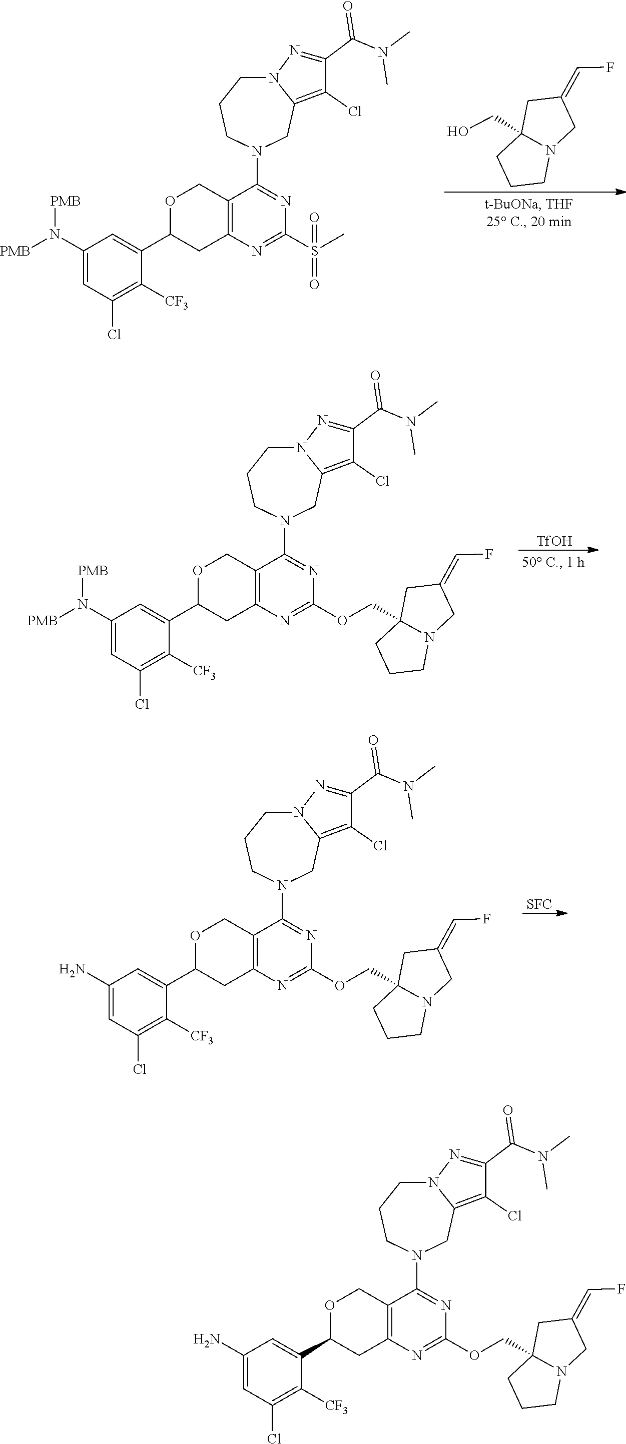

### Step 1: 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

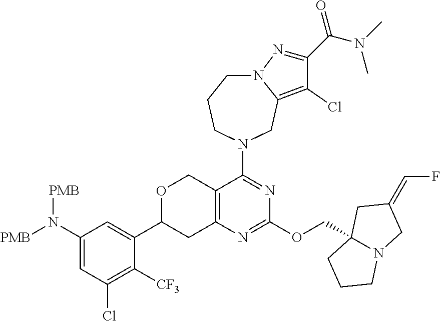

To a solution of 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (300 mg, 0.33 mmol, 1.0 eq), (S,Z)-(2-(fluoromethylene)tetrahydro-1H-pyrrolizin-7a(5H)-yl)methanol (140 mg, 0.68 mmol, 2.0 eq, hydrochloride) in tetrahydrofuran (5 mL) was added sodium t-butanolate (97 mg, 1.01 mmol, 3.0 eq), and the mixture was stirred at 25° C. for 20 min. LCMS showed the reaction was completed. The reaction mixture was quenched by adding saturated aqueous ammonium chloride (30 mL) and extracted with ethyl acetate (15 mL×2), washed with brine (20 mL), dried over anhydrous sodium sulfate, filtered and then concentrated in vacuum to give a residue. The residue was purified by prep-TLC (dichloromethane/methanol=9/1) to afford 5-(7-(5-(bis(4-methoxybenzyl)amino)-3-chloro-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (130 mg, 0.13 mmol, 40% yield) as a light yellow solid. LCMS (ESI, m/z): 979.3 [M+H]⁺.

### Step 2: 5-(7-(5-amino-3-chloro-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

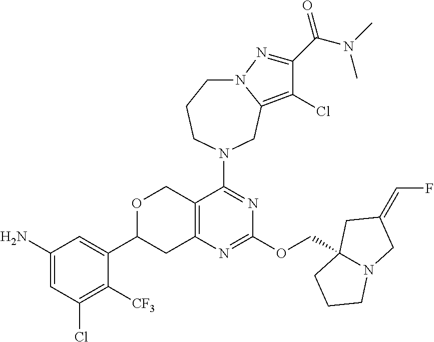 [SMILES: CN(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2]

A solution of 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (130 mg, 0.13 mmol, 1.0 eq) in trifluoromethanesulfonic acid (3.39 g, 22.60 mmol, 4 mL, 56.8 eq) was stirred at 50° C. for 1 h. LCMS showed the reaction was completed. The reaction mixture was quenched by adding saturated aqueous sodium bicarbonate (20 mL) and extracted with ethyl acetate (15 mL×2), dried over anhydrous sodium sulfate, filtered and then concentrated in vacuum to give a residue. The residue was purified by prep-HPLC (column: YMC-Actus Triart C18 150*30 mm*7 um; mobile phase: [water(FA)-ACN]; gradient:25%-55% B over 10 min) to afford 5-(7-(5-amino-3-chloro-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (50 mg, 0.067 mmol, 17% yield) as a white solid. LCMS (ESI, m/z): 739.3 [M+H]⁺. ¹H NMR (400 MHz, DMSO-d₆) δ: 6.88 (s, 1H), 6.84 (br s, 1H), 6.70 (d, J=2.0 Hz, 1H), 6.25-6.17 (m, 2H), 5.09-5.00 (m, 2H), 4.91-4.82 (m, 1H), 4.75-4.64 (m, 2H), 4.47-4.33 (m, 2H), 3.97-3.78 (m, 4H), 3.77-3.62 (m, 3H), 2.98 (s, 3H), 2.96 (s, 3H), 2.91-2.68 (m, 2H), 2.68-2.54 (m, 2H), 2.30-2.14 (m, 2H), 2.12-1.98 (m, 1H), 1.93-1.81 (m, 2H), 1.78-1.61 (m, 2H).

### Step 3: 5-((S)-7-(5-amino-3-chloro-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (Example 235)

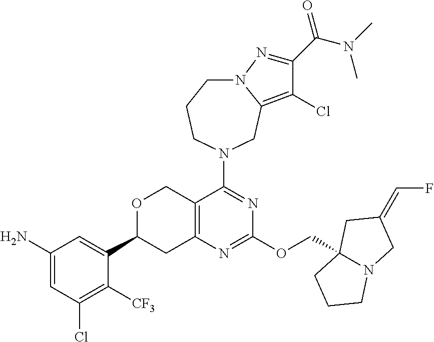 [SMILES: CN(C)C(=O)c1nn2c(c1Cl)CN(c1nc(OC[C@@]34CCCN3C/C(=C\F)C4)nc3c1CO[C@H](c1cc(N)cc(Cl)c1C(F)(F)F)C3)CCC2]

The mixture of diastereomers 5-(7-(5-amino-3-chloro-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (50 mg, 0.067 mmol, 1.0 eq) was separated by SFC (column: REGIS(S,S)WHELK-O1(250 mm*25 mm, 10 um); mobile phase: [CO₂-ACN/i-PrOH (0.1% NH3H2O)]; B %:60%, isocratic elution mode). The second eluent was identified as the desired diastereomer 5-((S)-7-(5-amino-3-chloro-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide (18.34 mg, 0.024 mmol, 36% yield, 98% purity, t_{R}=2.279 min) as an off-white solid. LCMS (ESI, m/z): 739.3 [M+H]⁺. ¹H NMR (400 MHz, DMSO-d₆) δ: 6.88 (s, 1H), 6.84 (br s, 1H), 6.70 (d, J=2.0 Hz, 1H), 6.22 (s, 2H), 5.10-5.00 (m, 2H), 4.88 (br d, J=16.8 Hz, 1H), 4.74-4.63 (m, 2H), 4.48-4.35 (m, 2H), 3.95-3.88 (m, 1H), 3.88-3.81 (m, 2H), 3.78-3.68 (m, 1H), 3.65 (br d, J=14.8 Hz, 1H), 2.98 (s, 3H), 2.96 (s, 3H), 2.90-2.82 (m, 1H), 2.70-2.59 (m, 1H), 2.45 (s, 1H), 2.35-2.22 (m, 2H), 2.22-2.14 (m, 1H), 2.11-1.94 (m, 2H), 1.93-1.76 (m, 3H), 1.76-1.64 (m, 2H).

### Example 247a: 5-((S)-7-(3-amino-5-chloro-2-fluoro-6-(trifluoromethyl)phenyl)-2-(((S,Z)-2-(fluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

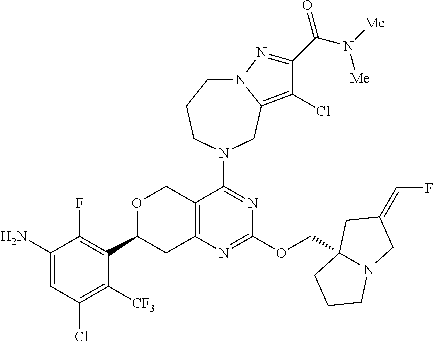 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2]

The title compound was prepared analogously to Example 213a, where ((2R,7aS)-2-fluorotetrahydro-1H-pyrrolizin-7a(5H)-yl)methanol was replaced with (S,Z)-(2-(fluoromethylene)tetrahydro-1H-pyrrolizin-7a(5H)-yl)methanol. The title compound was isolated as a white solid after purification of the mixture of 5-((S)-7-(3-amino-5-chloro-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((R)-7-(3-amino-5-chloro-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralpak IK 250 nm*30 mm, 10 m; isocratic elution using 40% acetonitrile/methanol (containing 0.1% of NH₄OH) in CO₂). MS (ESI) m/z: 757.2 [M+H]⁺. Analytical SFC: Retention time=1.30 minutes. Chiralpak IK-3 50*4.6 mm, ID=3 μm, temperature=35° C. Solvent: 40% methanol/acetonitrile (containing 0.05% of diethyl amine) in CO2; flow rate=3 mL/min; runtime=3.0 minutes; detector=PDA. ¹H NMR (400 MHz, CDCl3) δ 6.84 (d, J=8.0 Hz, 1H), 6.62-6.37 (m, 1H), 5.21 (dd, J=4.2, 11.6 Hz, 1H), 4.99 (br d, J=13.2 Hz, 1H), 4.84 (d, J=16.8 Hz, 1H), 4.69 (d, J=13.6 Hz, 1H), 4.55-4.47 (m, 1H), 4.45-4.33 (m, 2H), 4.18 (s, 2H), 4.08-3.98 (m, 2H), 3.97-3.91 (m, 1H), 3.83 (br d, J=15.2 Hz, 1H), 3.72-3.62 (m, 1H), 3.43-3.28 (m, 2H), 3.18-3.12 (m, 1H), 3.10 (s, 6H), 2.98 (br dd, J=4.0, 18.0 Hz, 1H), 2.71-2.59 (m, 2H), 2.36-2.27 (m, 2H), 2.13 (ddd, J=4.8, 7.6, 12.4 Hz, 2H), 1.94-1.87 (m, 2H), 1.79 (br d, J=8.0 Hz, 1H)

### Example 247b: 5-((R)-7-(3-amino-5-chloro-2-fluoro-6-(trifluoromethyl)phenyl)-2-(((S,Z)-2-(fluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

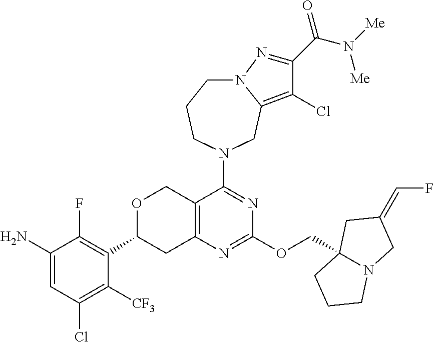 [SMILES: CN(C)C(=O)c1nn2c(c1Cl)CN(c1nc(OC[C@@]34CCCN3C/C(=C\F)C4)nc3c1CO[C@@H](c1c(F)c(N)cc(Cl)c1C(F)(F)F)C3)CCC2]

The title compound was prepared analogously to Example 213a, where ((2R,7aS)-2-fluorotetrahydro-1H-pyrrolizin-7a(5H)-yl)methanol was replaced with (S,Z)-(2-(fluoromethylene)tetrahydro-1H-pyrrolizin-7a(5H)-yl)methanol. The title compound was isolated as a white solid after purification of the mixture of 5-((S)-7-(3-amino-5-chloro-2-fluoro-6-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((R)-7-(3-amino-5-chloro-2-fluoro-6-(trifluoromethyl)phenyl)-2-(((S,Z)-2-(fluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralpak IK 250 nm*30 mm, 10 m; isocratic elution using 40% acetonitrile/methanol (containing 0.1% of NH₄OH) in CO₂). MS (ESI) m/z: 757.2 [M+H]⁺. Analytical SFC: Retention time=1.12 minutes. Chiralpak IK-3 50*4.6 mm, ID=3 μm, temperature=35° C. Solvent: 40% methanol/acetonitrile (containing 0.05% of diethyl amine) in CO2; flow rate=3 mL/min; runtime=3.0 minutes; detector=PDA. ¹H NMR (400 MHz, CDCl3) δ 6.84 (d, J=8.0 Hz, 1H), 6.63-6.33 (m, 1H), 5.21 (br dd, J=4.0, 10.8 Hz, 1H), 4.99 (br d, J=13.2 Hz, 1H), 4.85 (d, J=16.4 Hz, 1H), 4.68 (d, J=13.6 Hz, 1H), 4.55-4.48 (m, 1H), 4.41 (d, J=16.4 Hz, 1H), 4.38-4.32 (m, 1H), 4.18 (s, 2H), 4.09-3.92 (m, 3H), 3.84 (br d, J=15.2 Hz, 1H), 3.65 (ddd, J=3.2, 10.8, 13.8 Hz, 1H), 3.44-3.29 (m, 2H), 3.18-3.12 (m, 1H), 3.10 (s, 6H), 3.03-2.95 (m, 1H), 2.73-2.60 (m, 2H), 2.37-2.27 (m, 2H), 2.18-2.07 (m, 2H), 1.93-1.85 (m, 2H), 1.77 (br d, J=4.8 Hz, 1H)

### Example 248a: 5-((S)-7-(3-amino-5-chloro-2-fluoro-6-(trifluoromethyl)phenyl)-2-(((S)-2-(difluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

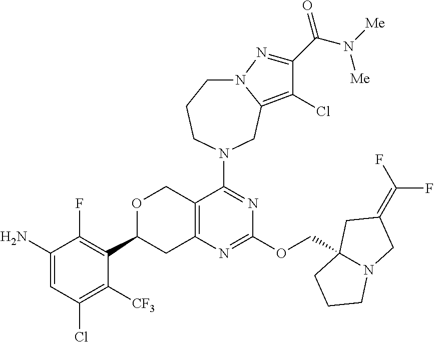 [SMILES: CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2]

The title compound was prepared analogously to Example 213a, where ((2R,7aS)-2-fluorotetrahydro-1H-pyrrolizin-7a(5H)-yl)methanol was replaced with (S)-(2-(difluoromethylene)tetrahydro-1H-pyrrolizin-7a(5H)-yl)methanol. The title compound was isolated as a white solid after purification of the mixture of 5-((S)-7-(3-amino-5-chloro-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((R)-7-(3-amino-5-chloro-2-fluoro-6-(trifluoromethyl)phenyl)-2-(((S)-2-(difluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralpak AD 250 nm*30 mm, 10 m; isocratic elution using 35% isopropanol (containing 0.1% of NH4OH) in CO₂). MS (ESI) m/z: 775.3 [M+H]+. Analytical SFC: Retention time=1.46 minutes. Chiralpak AD-3 50*4.6 mm, ID=3 μm, temperature=35° C. Solvent: 20-60% methanol (containing 0.05% of diethyl amine) in CO₂; flow rate=3 mL/min; runtime=3.0 minutes; detector=PDA. ¹H NMR (400 MHz, CDCl3) δ 6.84 (d, J=7.6 Hz, 1H), 5.20 (br dd, J=3.6, 11.2 Hz, 1H), 4.98 (d, J=13.6 Hz, 1H), 4.84 (d, J=16.4 Hz, 1H), 4.69 (d, J=13.6 Hz, 1H), 4.56-4.47 (m, 1H), 4.43 (d, J=16.4 Hz, 1H), 4.39-4.31 (m, 1H), 4.19 (s, 2H), 4.10-4.04 (m, 1H), 4.03-3.97 (m, 1H), 3.97-3.89 (m, 1H), 3.76-3.61 (m, 2H), 3.41-3.28 (m, 2H), 3.17-3.12 (m, 1H), 3.10 (s, 6H), 2.98 (br dd, J=3.6, 18.0 Hz, 1H), 2.76 (br d, J=15.2 Hz, 1H), 2.67-2.58 (m, 1H), 2.41-2.30 (m, 2H), 2.19-2.11 (m, 2H), 1.96-1.88 (m, 2H), 1.87 (br s, 1H)

### Example 248b: 5-((R)-7-(3-amino-5-chloro-2-fluoro-6-(trifluoromethyl)phenyl)-2-(((S)-2-(difluoromethylene)tetrahydro-1H-pyrrolizin-7a(5H)-yl)methoxy)-7, 8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide

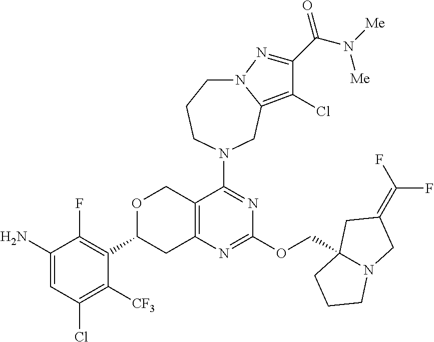 [SMILES: CN(C)C(=O)c1nn2c(c1Cl)CN(c1nc(OC[C@@]34CCCN3CC(=C(F)F)C4)nc3c1CO[C@@H](c1c(F)c(N)cc(Cl)c1C(F)(F)F)C3)CCC2]

The title compound was prepared analogously to Example 213a, where ((2R,7aS)-2-fluorotetrahydro-1H-pyrrolizin-7a(5H)-yl)methanol was replaced with (S)-(2-(difluoromethylene)tetrahydro-1H-pyrrolizin-7a(5H)-yl)methanol. The title compound was isolated as a white solid after purification of the mixture of 5-((S)-7-(3-amino-5-chloro-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide and 5-((R)-7-(3-amino-5-chloro-2-fluoro-6-(trifluoromethyl)phenyl)-2-(((S)-2-(difluoromethylene)tetrahydro-1H-pyrrolizin-7a(5H)-yl)methoxy)-7,8-dihydro-5H-pyrano[4,3-d]pyrimidin-4-yl)-3-chloro-N,N-dimethyl-5,6,7,8-tetrahydro-4H-pyrazolo[1,5-a][1,4]diazepine-2-carboxamide by SFC (DAICEL Chiralpak AD 250 nm*30 mm, 10 m; isocratic elution using 35% isopropanol (containing 0.1% of NH₄OH) in CO₂). MS (ESI) m/z: 775.3 [M+H]+. Analytical SFC: Retention time=1.35 minutes. Chiralpak AD-3 50*4.6 mm, ID=3 μm, temperature=35° C. Solvent: 20-60% methanol (containing 0.05% of diethyl amine) in CO₂; flow rate=3 mL/min; runtime=3.0 minutes; detector=PDA. ¹H NMR (400 MHz, CDCl3) δ 6.84 (d, J=7.6 Hz, 1H), 5.26-5.17 (m, 1H), 4.97 (br d, J=13.2 Hz, 1H), 4.85 (br d, J=16.4 Hz, 1H), 4.68 (d, J=13.6 Hz, 1H), 4.55-4.47 (m, 1H), 4.42 (br d, J=16.8 Hz, 1H), 4.39-4.32 (m, 1H), 4.18 (s, 2H), 4.10-3.99 (m, 2H), 3.96-3.88 (m, 1H), 3.79-3.61 (m, 2H), 3.40-3.28 (m, 2H), 3.17-3.12 (m, 1H), 3.10 (s, 6H), 3.02-2.94 (m, 1H), 2.77 (br d, J=15.6 Hz, 1H), 2.66-2.58 (m, 1H), 2.41-2.29 (m, 2H), 2.20-2.08 (m, 2H), 1.95-1.87 (m, 2H), 1.82 (br d, J=8.4 Hz, 1H).

### 5-((S)-7-(5-amino-3-chloro-2-(trifluoromethyl)phenyl)-2-(((S,Z)-2-(fluoromethylene)tetr ahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide

### Example 300

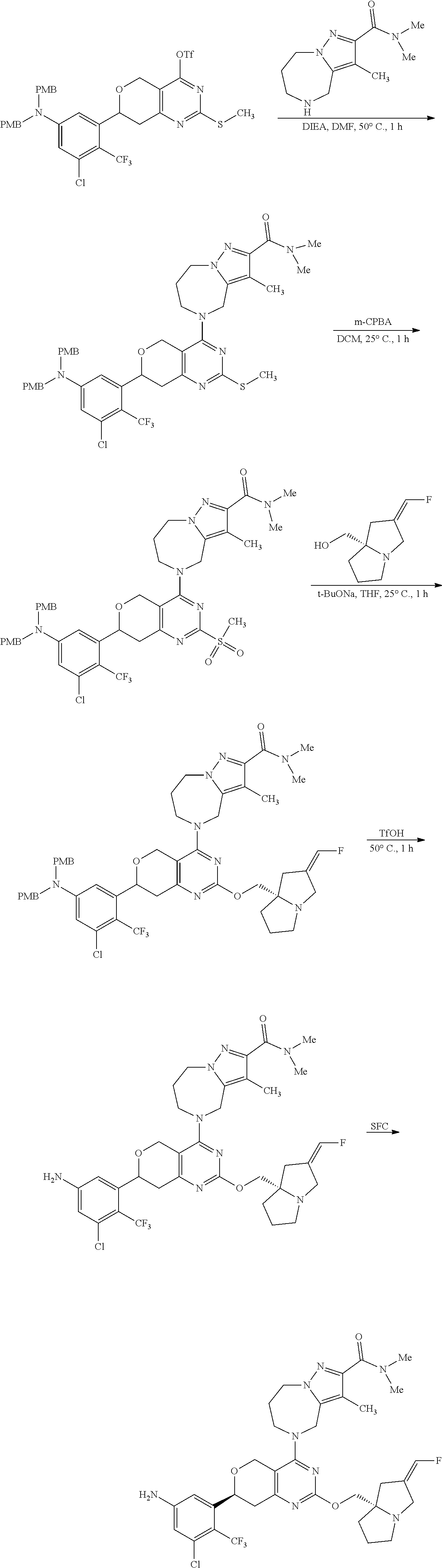

### Step 1: 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide

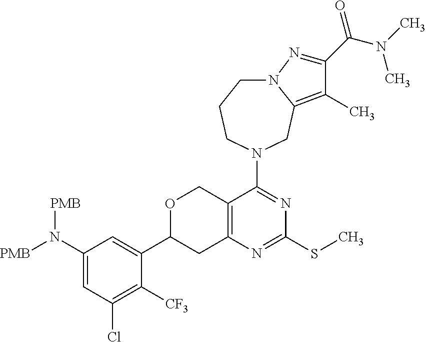

To a mixture of 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl trifluoromethanesulfonate (500 mg, 0.65 mmol, 1.0 eq) and N,N,3-trimethyl-5,6,7,8-tetrahydro-4H-pyrazolo[1,5-a][1,4]diazepine-2-carboxamide (264 mg, 0.79 mmol, 1.2 eq, trifluoroacetate) in dimethylformamide (5 mL) was added diisopropylethylamine (88 mg, 0.65 mmol, 0.11 mL, 1.0 eq). The mixture was stirred at 50° C. for 0.5 h. LCMS showed the reaction was completed. The mixture was diluted with water (40 mL) and extracted with ethyl acetate (30 mL×3). The combined organic layers were washed with brine (30 mL×3), dried over anhydrous sodium sulfate, filtered and concentrated under reduced pressure to give a residue. The residue was purified by prep-TLC (dichloromethane/methanol=10/1) to afford 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide. (440 mg, 0.52 mmol, 80% yield) as a white solid. LCMS (ESI, m/z): 836.2 [M+H]⁺.

### Step 2: 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide

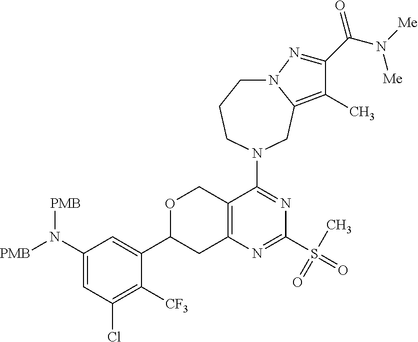

To a solution of 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (440 mg, 0.53 mmol, 1.0 eq) in dichloromethane (4 mL) was added m-chloroperoxybenzoic acid (214 mg, 1.0 mmol, 85% purity, 2.0 eq). The mixture was stirred at 25° C. for 0.5 h. LCMS showed the reaction was completed. The reaction mixture was quenched by adding saturated aqueous sodium bicarbonate (50 mL) and extracted with ethyl acetate (30 mL×3). The combined organic layers were washed with brine (40 mL×3), dried over anhydrous sodium sulfate, filtered and concentrated under reduced pressure. 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (450 mg, 0.52 mmol, 98% yield) was obtained as a white solid. LCMS (ESI, m/z): 852.3 [M+H]⁺.

### Step 3: 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide

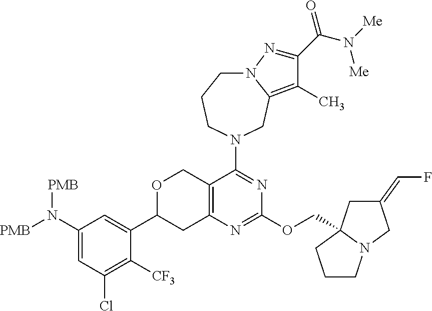

To a mixture of 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (450 mg, 0.52 mmol, 1.0 eq) and [(6Z,8S)-6-(fluoromethylene)-2,3,5,7-tetrahydro-1H-pyrrolizin-8-yl]methanol (129 mg, 0.62 mmol, 1.2 eq, hydrochloride) in tetrahydrofuran (5 mL) was added sodium t-butanolate (149 mg, 1.55 mmol, 3.0 eq). The mixture was stirred at 25° C. for 0.3 h. LCMS showed the reaction was completed. The mixture was diluted with water (20 mL) and extracted with ethyl acetate (10 mL×3). The combined organic layers were washed with brine (10 mL×3), dried over anhydrous sodium sulfate, filtered and concentrated under reduced pressure to give a residue. The residue was purified by prep-TLC (dichloromethane/methanol=10/1) to afford 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (250 mg, 0.26 mmol, 50% yield) as a white solid. LCMS (ESI, m/z): 959.4 [M+H]⁺.

### Step 4: 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a]1[1,4]diazepine-2-carboxamide

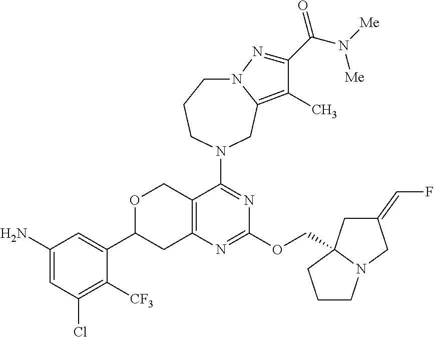 [SMILES: Cc1c(C(=O)N(C)C)nn2c1CN(c1nc(OC[C@@]34CCCN3C/C(=C\F)C4)nc3c1COC(c1cc(N)cc(Cl)c1C(F)(F)F)C3)CCC2]

A solution of 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (250 mg, 0.26 mmol, 1.0 eq) in trifluoromethanesulfonic acid (3 mL) was stirred at 50° C. for 1 h. LCMS showed the reaction was completed. The reaction mixture was filtered and concentrated under reduced pressure to give a residue. The residue was purified by prep-HPLC (column: YMC-Actus Triart C18 150×30 mm×7 um; mobile phase: [water(FA)-ACN]; gradient:23%-53% B over 10 min) to afford 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a]1[1,4]diazepine-2-carboxamide (83 mg, 0.12 mmol, 44% yield) as a white solid. LCMS (ESI, m/z): 719.2 [M+H]⁺. ¹H NMR (400 MHz, DMSO-d₆) δ: 7.11-6.80 (m, 2H), 6.69 (d, J=2.0 Hz, 1H), 6.21 (s, 2H), 5.04 (br d, J=12.4 Hz, 2H), 4.89-4.49 (m, 4H), 4.48-4.03 (m, 5H), 3.90-3.78 (m, 2H), 3.35 (br s, 2H), 3.18-3.03 (m, 4H), 2.94 (s, 3H), 2.85 (br d, J=17.6 Hz, 1H), 2.64 (br dd, J=11.6, 17.4 Hz, 3H), 2.18-2.00 (m, 6H), 1.98-1.89 (m, 2H).

### Step 5: 5-((S)-7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a]1[1,4]diazepine-2-carboxamide (Example 300)

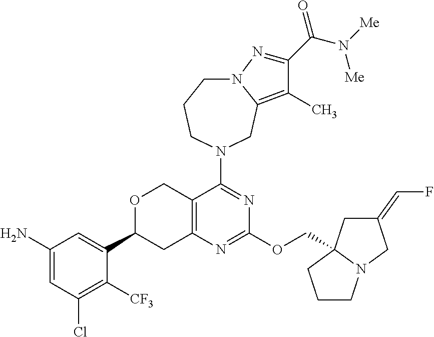 [SMILES: Cc1c(C(=O)N(C)C)nn2c1CN(c1nc(OC[C@@]34CCCN3C/C(=C\F)C4)nc3c1CO[C@H](c1cc(N)cc(Cl)c1C(F)(F)F)C3)CCC2]

The mixture of diastereomers 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7, 8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (79 mg, 0.11 mmol, 1.0 eq) was separated by SFC (column: DAICEL CHIRALCEL OX (250 mm*30 mm, 0 um); mobile phase: [CO₂-ACN/i-PrOH (0.1% NH₃H2O)]; B %:60%, isocratic elution mode), and the first eluent was identified as the desired diastereomer 5-((S)-7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N,3-trimethyl-5,6,7,8-tetrahydro-4H-pyrazolo[1,5-a][1,4]diazepine-2-carboxamide (23 mg, 0.03 mmol, 28% yield, 98% purity) was obtained as a yellow solid. LCMS (ESI, m/z): 719.4 [M+H]⁺. ¹H NMR (400 MHz, DMSO-d₆) δ: 6.90-6.61 (m, 3H), 6.21 (s, 2H), 5.08-4.96 (m, 2H), 4.84-4.57 (m, 3H), 4.46-4.31 (m, 2H), 3.89-3.76 (m, 4H), 3.65 (br d, J=15.2 Hz, 1H), 3.33-3.23 (m, 3H), 3.06 (s, 3H), 2.93 (s, 5H), 2.62 (br dd, J=10.8, 17.4 Hz, 1H), 2.45 (s, 1H), 2.26 (br d, J=16.4 Hz, 1H), 2.11 (s, 3H), 2.04-1.95 (m, 1H), 1.91-1.79 (m, 2H), 1.78-1.61 (m, 2H).

### 5-((S)-7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (Example 301)

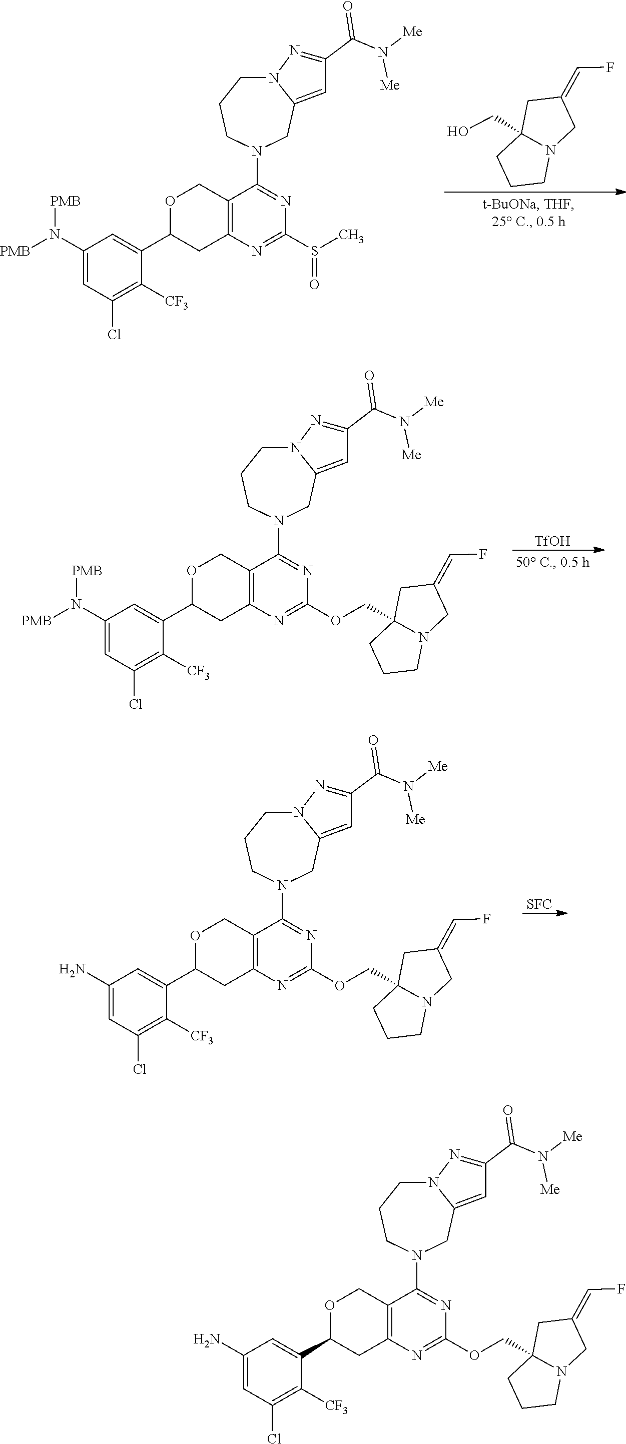

### Step 1: 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide

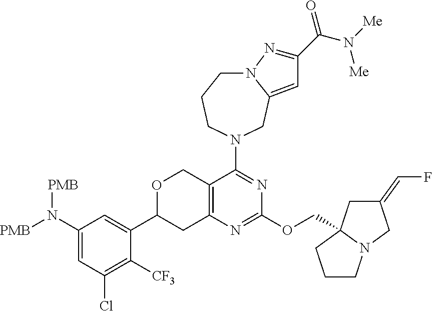

To a solution of 5-(7-(5-(bis(4-methoxybenzyl)amino)-3-chloro-2-(trifluoromethyl)phenyl)-2-(methylsulfinyl)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (340 mg, 0.40 mmol, 1.0 eq) and (S,Z)-(2-(fluoromethylene)tetrahydro-1H-pyrrolizin-7a(5H)-yl)methanol (168 mg, 0.81 mmol, 2.0 eq, hydrochloride) in tetrahydrofuran (6 mL) was added sodium tert-butoxide (117 mg, 1.22 mmol, 3.0 eq). The mixture was stirred at 25° C. for 0.5 h. LCMS showed the reaction was completed. The reaction mixture was quenched by saturated aqueous ammonium chloride (50 mL), and then extracted with ethyl acetate (15 mL×3). The combined organic layers were washed with brine (20 mL×2), dried over anhydrous sodium sulfate, filtered and concentrated under reduced pressure to give a residue. The residue was purified by prep-TLC (dichloromethane/methanol=10/1) to afford 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (220 mg, 0.23 mmol, 57% yield) as a yellow solid. LCMS (ESI, m/z): 945.5 [M+H]⁺.

### Step 2: 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide

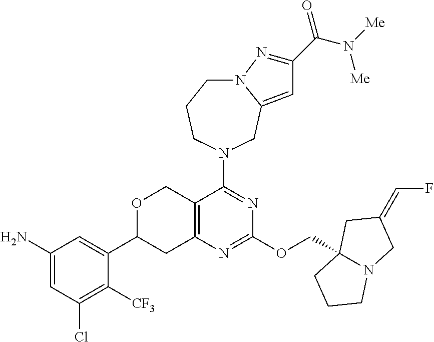 [SMILES: CN(C)C(=O)c1cc2n(n1)CCCN(c1nc(OC[C@@]34CCCN3C/C(=C\F)C4)nc3c1COC(c1cc(N)cc(Cl)c1C(F)(F)F)C3)C2]

A solution of 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (220 mg, 0.23 mmol, 1.0 eq) in trifluoromethanesulfonic acid (5 mL) was stirred at 50° C. for 0.5 h. LCMS showed the reaction was completed. The reaction mixture was diluted with ethyl acetate (30 mL), and then quenched by saturated aqueous sodium bicarbonate (100 mL). The mixture was extracted with ethyl acetate (30 mL×3). The combined organic layers were washed with brine (50 mL×2), dried over anhydrous sodium sulfate, filtered and concentrated under reduced pressure to give a residue. The residue was purified by prep-HPLC (column: YMC-Actus Triart C18 150*30 mm*7 um; mobile phase: [water(FA)-ACN]; gradient:23%-53% B over 10 min) to afford 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (73.3 mg, 0.10 mmol, 44.67% yield) as an off-white solid. LCMS (ESI, m/z): 705.4 [M+H]⁺. ¹H NMR (400 MHz, DMSO-d₆) δ: 6.92 (br s, 1H), 6.89 (d, J=1.6 Hz, 1H), 6.73-6.70 (m, 1H), 6.69 (d, J=2.0 Hz, 1H), 6.60 (s, 1H), 6.20 (s, 2H), 5.04 (br d, J=13.6 Hz, 2H), 4.77-4.67 (m, 3H), 4.45 (br s, 2H), 4.20-3.95 (m, 2H), 3.85 (br s, 3H), 3.24 (s, 3H), 2.93 (s, 3H), 2.88-2.74 (m, 2H), 2.67-2.58 (m, 2H), 2.48-2.32 (m, 2H), 2.24-2.05 (m, 2H), 2.02-1.89 (m, 3H), 1.88-1.71 (m, 2H).

### Step 3: 5-((S)-7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (Example 301)

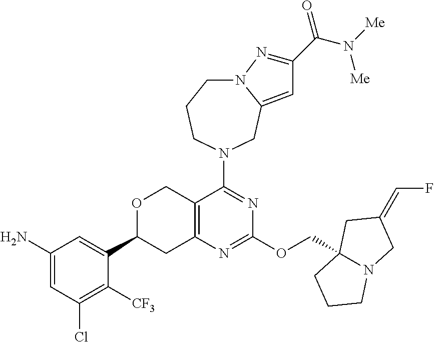 [SMILES: CN(C)C(=O)c1cc2n(n1)CCCN(c1nc(OC[C@@]34CCCN3C/C(=C\F)C4)nc3c1CO[C@H](c1cc(N)cc(Cl)c1C(F)(F)F)C3)C2]

The mixture of diastereomers 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][11,4]diazepine-2-carboxamide (70 mg, 0.10 mmol, 1.0 eq) was separated by SFC (column: (s,s) WHELK-O1 (250 mm*30 mm, 10 um); mobile phase: [CO₂-ACN/i-PrOH (0.1% NH3H2O)]; B %:58%, isocratic elution mode). The second eluent was identified as the desired diastereomer 5-((R)-7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (24.12 mg, 0.03 mmol, 34% yield, 100% purity, t_{R}=1.656 min) as an off-white solid. LCMS (ESI, m/z): 705.4 [M+H]⁺. ¹H NMR (400 MHz, DMSO-d₆) δ: 6.89 (d, J=2.0 Hz, 1H), 6.84 (br s, 1H), 6.69 (d, J=2.0 Hz, 1H), 6.62 (s, 1H), 6.57 (s, 1H), 6.20 (s, 2H), 5.04 (br d, J=12.8 Hz, 2H), 4.73-4.66 (m, 3H), 4.47-4.42 (m, 2H), 3.87-3.82 (m, 3H), 3.66 (br d, J=15.6 Hz, 1H), 3.24 (s, 3H), 2.93 (s, 3H), 2.90-2.78 (m, 2H), 2.73-2.59 (m, 2H), 2.34-2.24 (m, 2H), 2.18-2.06 (m, 2H), 1.98-1.81 (m, 4H), 1.75-1.63 (m, 2H).

### Synthesis of 5-((S)-7-(5-amino-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (Example 302)

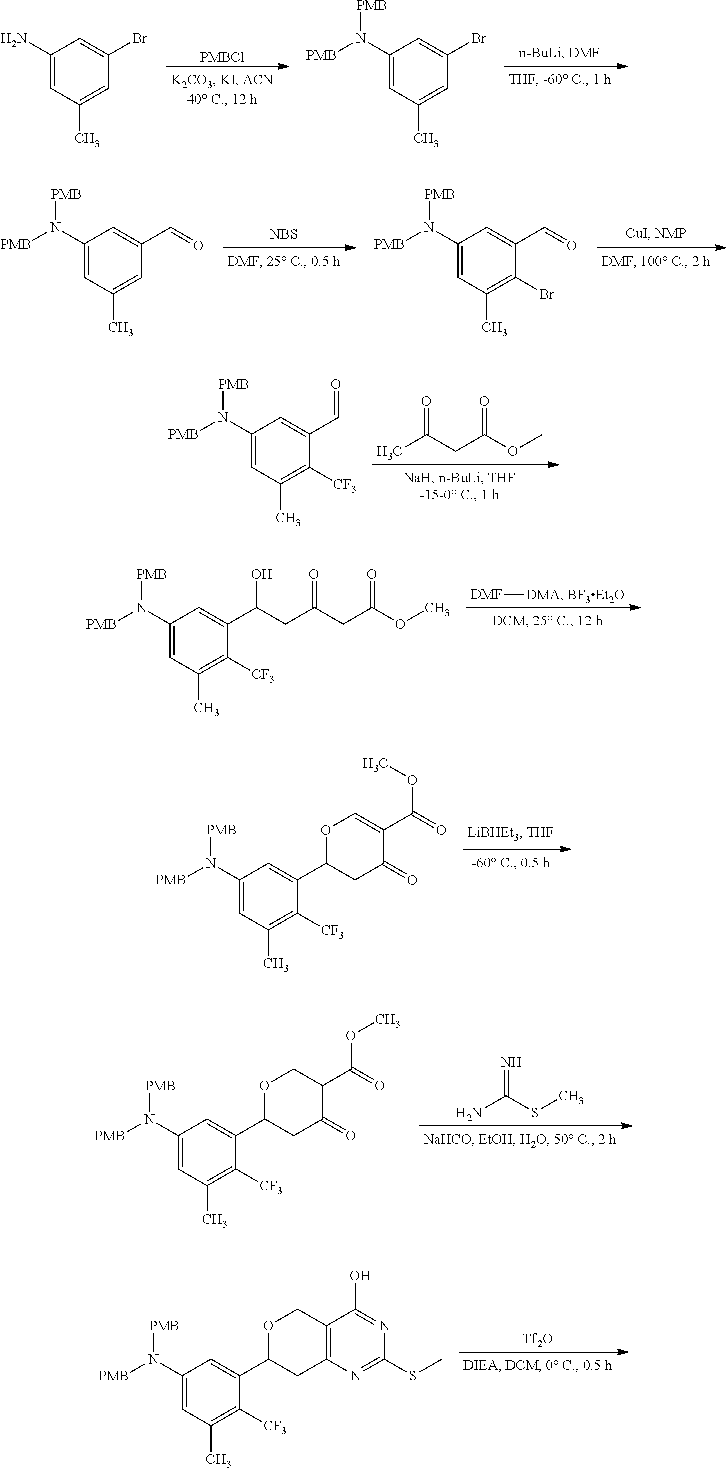

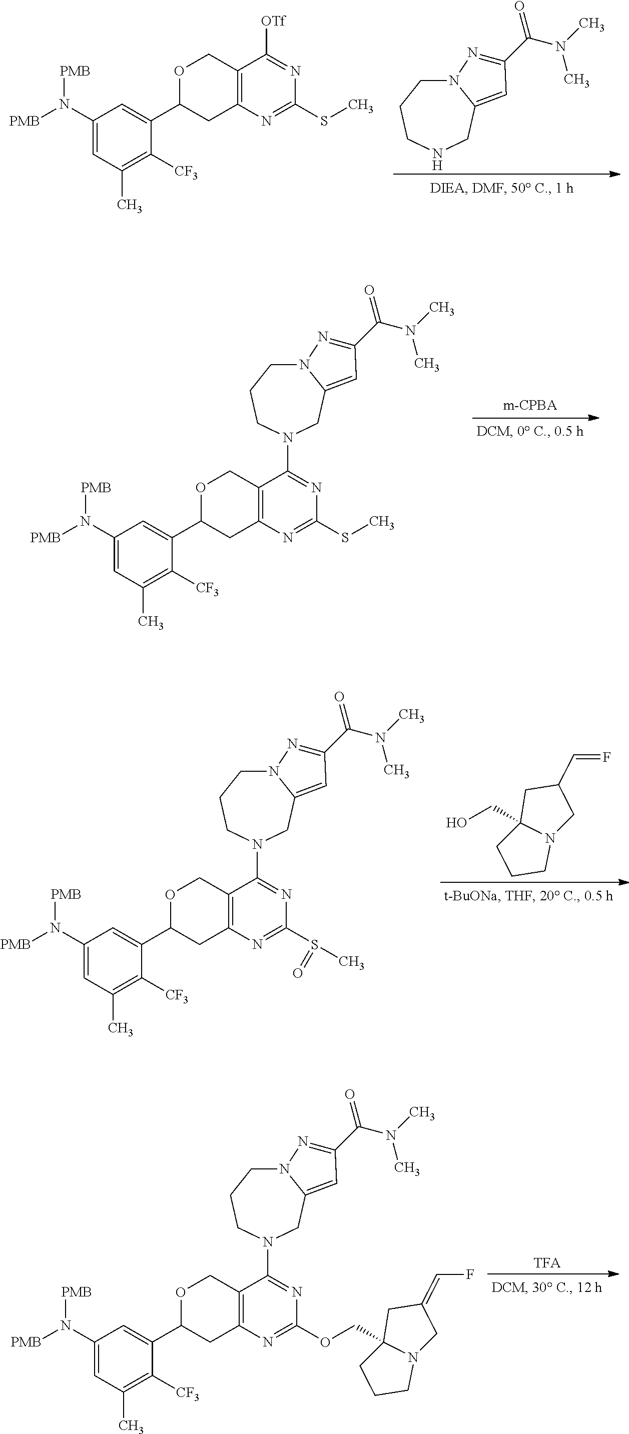

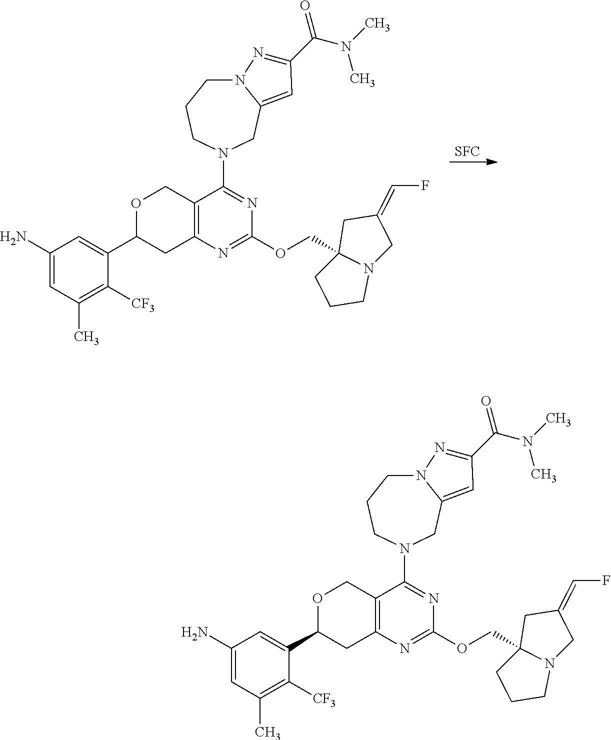

### Step 1: 3-bromo-N,N-bis(4-methoxybenzyl)-5-methylaniline

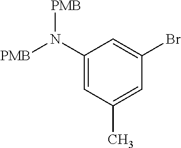

To a solution of 3-bromo-5-methylaniline (25 g, 134.37 mmol, 1.0 eq), potassium carbonate (56 g, 403.12 mmol, 3.0 eq) and potassium iodide (2 g, 13.44 mmol, 0.1 eq) in acetonitrile (300 mL) was added 1-(chloromethyl)-4-methoxy-benzene (44.19 g, 282.18 mmol, 2.1 eq). The mixture was stirred at 40° C. for 12 h. LCMS showed the desired mass was detected. The mixture was filtered and the filtrate was concentrated under reduced pressure to give a residue. The residue was purified by column chromatography on silica gel (Petroleum ether/Ethyl acetate=10/1 to 1/1) to afford 3-bromo-N,N-bis(4-methoxybenzyl)-5-methylaniline (220 g, crude) as a brown oil. LCMS (ESI, m/z): 428.1 [M+H]⁺.

### Step 2: 3-(bis(4-methoxybenzyl)amino)-5-methylbenzaldehyde

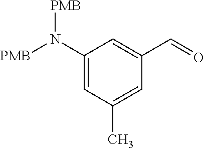

To a solution of 3-bromo-N,N-bis(4-methoxybenzyl)-5-methylaniline (15 g, 35.18 mmol, 1.0 eq) in tetrahydrofuran (150 mL) was added n-butyl lithium (2.5 M, 16.89 mL, 1.2 eq) at −60° C. in nitrogen. The mixture was stirred at −60° C. for 30 min. Then a solution of N,N-dimethylformamide (3.09 g, 42.22 mmol, 3.25 mL, 1.2 eq) in tetrahydrofuran (10 mL) was added at −60° C., and the mixture was stirred at −60° C. for 30 min. LCMS showed the desired mass was detected. The reaction mixture was quenched by water (40 mL) at 0° C., and then extracted with ethyl acetate (100 mL×2). The combined organic layers were washed with brine (30 mL×3), dried over anhydrous sodium sulfate, filtered and concentrated under reduced pressure to give a residue. The residue was purified by column chromatography on silica gel (Petroleum ether/Ethyl acetate=100/1 to 3/1) to afford 3-(bis(4-methoxybenzyl)amino)-5-methylbenzaldehyde (15 g, crude) as a yellow solid. LCMS (ESI, m/z): 398.2 [M+H]⁺, ¹H NMR (400 MHz, DMSO-d₆) δ: 9.78 (s, 1H), 7.16 (d, J=8.4 Hz, 4H), 6.99 (s, 1H), 6.95 (s, 1H), 6.90-6.86 (m, 5H), 4.62 (s, 4H), 3.71 (s, 6H), 2.23 (s, 3H).

### Step 3: 5-(bis(4-methoxybenzyl)amino)-2-bromo-3-methylbenzaldehyde

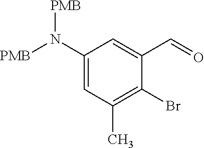

To a solution of 3-(bis(4-methoxybenzyl)amino)-5-methylbenzaldehyde (25 g, 66.59 mmol, 1.0 eq) in N,N-dimethylformamide (100 mL) was added 1-bromopyrrolidine-2,5-dione (13.04 g, 73.24 mmol, 1.1 eq). The mixture was stirred at 25° C. for 0.5 h. LCMS showed the desired mass was detected. The reaction mixture was diluted with water (400 mL) and extracted with ethyl acetate (150 mL×3). The combined organic layers were washed with brine (100 mL×3), dried over anhydrous sodium sulfate, filtered and concentrated under reduced pressure to give a residue. The residue was triturated using a mixed solvent of petroleum ether and ethyl acetate (10/1, 100 mL) at 25° C. for 10 min to afford 5-(bis(4-methoxybenzyl)amino)-2-bromo-3-methylbenzaldehyde (35 g, crude) as a yellow solid. LCMS (ESI, m/z): 456.1 [M+H]⁺, ¹H NMR (400 MHz, DMSO-d₆) δ: 10.16 (s, 1H), 7.15 (d, J=8.4 Hz, 4H), 7.09 (d, J=3.2 Hz, 1H), 6.93 (d, J=3.2 Hz, 1H), 6.89 (s, 4H), 4.62 (s, 4H), 3.72 (s, 6H), 2.29 (s, 3H).

### Step 4: 5-(bis(4-methoxybenzyl)amino)-3-methyl-2-(trifluoromethyl)benzaldehyde

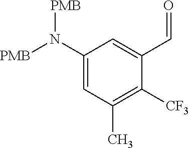

A mixture of 5-(bis(4-methoxybenzyl)amino)-2-bromo-3-methylbenzaldehyde (2.3 g, 5.06 mmol, 1.0 eq), methyl 2,2-difluoro-2-fluorosulfonyl-acetate (4.86 g, 25.31 mmol, 3.22 mL, 5.0 eq), cuprous iodide (2.41 g, 12.66 mmol, 2.5 eq) in N,N-dimethylformamide (20 mL) was degassed and purged with nitrogen 3 times, and then the mixture was stirred at 100° C. for 2 h in nitrogen. TLC showed the 5-(bis(4-methoxybenzyl) amino)-2-bromo-3-methylbenzaldehyde was consumed completely and one new spot was formed. The reaction mixture was filtered, diluted with water (100 mL) and extracted with ethyl acetate (50 mL×3). The combined organic layers were washed with brine (30 mL×3), dried over anhydrous sodium sulfate, filtered and concentrated under reduced pressure to give a residue. The residue was purified by flash silica gel chromatography (ISCO®; 12 g SepaFlash® Silica Flash Column, Eluent of 0~20% Ethyl acetate/Petroleum ether gradient @100 mL/min) to afford 5-(bis(4-methoxybenzyl)amino)-3-methyl-2-(trifluoromethyl)benzaldehyde (2 g, 4.51 mmol, 89% yield) as a yellow solid. ¹H NMR (400 MHz, DMSO-d₆) δ: 10.22-10.14 (m, 1H), 7.19-7.13 (m, 4H), 6.95-6.88 (m, 6H), 4.70 (s, 4H), 3.72 (s, 6H), 2.38-2.32 (m, 3H).

### Step 5: methyl 5-(5-(bis(4-methoxybenzyl)amino)-3-methyl-2-(trifluoromethyl)phenyl)-5-hydroxy-3-oxopentanoate

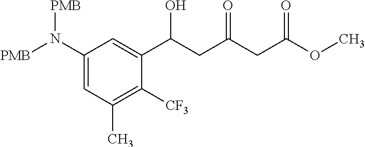

To a solution of methyl 3-oxobutanoate (3.27 g, 28.19 mmol, 2.5 eq) in tetrahydrofuran (30 mL) was added sodium hydride (1.13 g, 28.19 mmol, 60% purity, 2.5 eq) and n-butyl lithium (2.5 M, 11.28 mL, 2.5 eq) at 0° C. The mixture was stirred at 0° C. for 0.5 h, then the mixture was cooled to −15° C., and 5-(bis(4-methoxybenzyl)amino)-3-methyl-2-(trifluoromethyl) benzaldehyde (5 g, 11.28 mmol, 1.0 eq) in tetrahydrofuran (20 mL) was added dropwise. The mixture was stirred at −15° C. for 0.5 h. LCMS showed the desired mass was detected. The reaction mixture was quenched by saturated aqueous ammonium chloride (50 mL), and extracted with ethyl acetate (50 mL×3). The combined organic layers were washed with brine (50 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0~35% Ethyl acetate/Petroleum ether gradient @150 mL/min) to afford methyl 5-(5-(bis(4-methoxybenzyl)amino)-3-methyl-2-(trifluoromethyl)phenyl)-5-hydroxy-3-oxopentanoate (9.8 g, crude) as a yellow oil. LCMS (ESI, m/z): 560.3 [M+H]⁺, ¹H NMR (400 MHz, DMSO-d₆) δ: 7.16 (d, J=8.8 Hz, 4H), 7.07 (d, J=2.0 Hz, 1H), 6.89 (d, J=8.8 Hz, 4H), 6.56 (d, J=1.6 Hz, 1H), 5.75 (s, 1H), 5.45 (d, J=4.4 Hz, 1H), 5.35-5.24 (m, 1H), 4.66-4.56 (m, 4H), 3.72 (s, 6H), 3.62 (s, 1H), 3.61 (s, 3H), 3.57 (d, J=5.6 Hz, 2H), 2.30-2.24 (m, 3H).

### Step 6: methyl 2-(5-(bis(4-methoxybenzyl)amino)-3-methyl-2-(trifluoromethyl)phenyl)-4-oxo-3,4-dihydro-2H-pyran-5-carboxylate

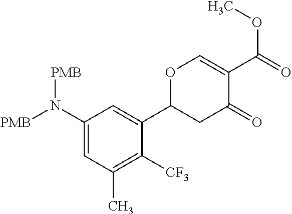

To a solution of methyl 5-[5-[bis[(4-methoxyphenyl)methyl]amino]-3-methyl-2-(trifluoromethyl) phenyl]-5-hydroxy-3-oxo-pentanoate (9.8 g, 17.51 mmol, 1.0 eq) in dichloromethane (100 mL) was added N,N-dimethylformamide dimethyl acetal (2.50 g, 21.02 mmol, 1.2 eq). The mixture was stirred at 25° C. for 12 h. Then boron trifluoride diethyl etherate (2.98 g, 21.02 mmol, 1.2 eq) was added, and the mixture was stirred at 25° C. for 1 h. LCMS showed the desired mass was detected. The reaction mixture was quenched by adding saturated aqueous sodium bicarbonate (50 mL) at 20° C., and then extracted with dichloromethane (50 mL×3). The combined organic layers were washed with brine (50 mL), dried over anhydrous sodium sulfate, filtered and concentrated under reduced pressure. Compound methyl 2-(5-(bis(4-methoxybenzyl)amino)-3-methyl-2-(trifluoromethyl)phenyl)-4-oxo-3,4-dihydro-2H-pyran-5-carboxylate (9.9 g, crude) was obtained as a yellow oil. LCMS (ESI, m/z): 570.3 [M+H]⁺.

### Step 7: methyl 6-(5-(bis(4-methoxybenzyl)amino)-3-methyl-2-(trifluoromethyl)phenyl)-4-oxotetrahydro-2H-pyran-3-carboxylate

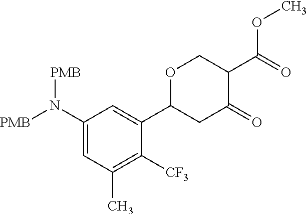

To a solution of methyl 2-(5-(bis(4-methoxybenzyl)amino)-3-methyl-2-(trifluoromethyl)phenyl)-4-oxo-3,4-dihydro-2H-pyran-5-carboxylate (9.9 g, 17.38 mmol, 1.0 eq) in tetrahydrofuran (100 mL) was added lithium triethylborohydride (1 M, 19.12 mL, 1.1 eq) at −60° C. The mixture was stirred at −60° C. for 0.5 h. LCMS showed the desired mass was detected. The reaction mixture was quenched by the addition of saturated aqueous ammonium chloride (40 mL) at 0° C., and then extracted with ethyl acetate (30 mL×3). The combined organic layers were washed with brine (40 mL), dried over anhydrous sodium sulfate, filtered and concentrated under reduced pressure. Methyl 6-(5-(bis(4-methoxybenzyl)amino)-3-methyl-2-(trifluoromethyl) phenyl)-4-oxotetrahydro-2H-pyran-3-carboxylate (10 g, crude) was obtained as a yellow oil. LCMS (ESI, m/z): 572.2 [M+H]⁺.

### Step 8: 7-(5-(bis(4-methoxybenzyl)amino)-3-methyl-2-(trifluoromethyl)phenyl)-2-(methylthio)-7,8-dihydro-5H-pyrano[4,3-d]pyrimidin-4-ol

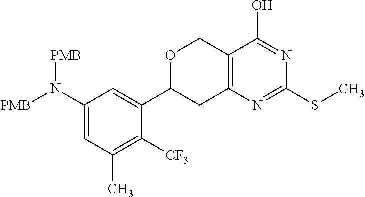

To a solution of methyl 6-(5-(bis(4-methoxybenzyl)amino)-3-methyl-2-(trifluoromethyl)phenyl)-4-oxotetrahydro-2H-pyran-3-carboxylate (10 g, 17.50 mmol, 1.0 eq) and 2-methylisothiourea; sulfuric acid (48.70 g, 174.95 mmol, 10.0 eq) in ethanol (100 mL) and water (30 mL) was added sodium bicarbonate (29.39 g, 349.91 mmol, 20.0 eq). The mixture was stirred at 50° C. for 2 h. LCMS showed the desired mass was detected. The reaction mixture was diluted with water (200 mL), and then extracted with ethyl acetate (100 mL×3). The combined organic layers were washed with brine (50 mL×3), dried over anhydrous sodium sulfate, filtered and concentrated under reduced pressure to give a residue. The residue was purified by prep-HPLC (column: Phenomenex luna C18 (250*70 mm, 10 um); mobile phase: [water(FA)-ACN]; gradient: 70%-100% B over 20 min). 7-(5-(bis(4-methoxybenzyl)amino)-3-methyl-2-(trifluoromethyl) phenyl)-2-(methylthio)-7,8-dihydro-5H-pyrano[4,3-d]pyrimidin-4-ol (2.06 g, 3.37 mmol, 19.25% yield) was obtained as a white solid. LCMS (ESI, m/z): 612.3 [M+H]⁺.

### Step 9: 7-(5-(bis(4-methoxybenzyl)amino)-3-methyl-2-(trifluoromethyl)phenyl)-2-(methylthio)-7,8-dihydro-5H-pyrano[4,3-d]pyrimidin-4-yl trifluoromethanesulfonate

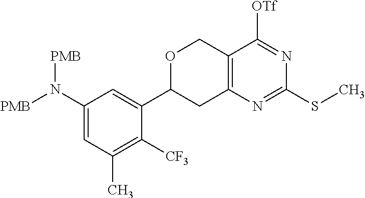

To a solution of 7-(5-(bis(4-methoxybenzyl)amino)-3-methyl-2-(trifluoromethyl)phenyl)-2-(methylthio)-7,8-dihydro-5H-pyrano[4,3-d]pyrimidin-4-ol (1.3 g, 2.13 mmol, 1.0 eq) in dichloromethane (15 mL) was added diisopropylethylamine (1.37 g, 10.63 mmol, 5.0 eq) and trifluoromethanesulfonic anhydride (1.20 g, 4.25 mmol, 2.0 eq) at 0° C. The mixture was stirred at 0° C. for 0.5 h. LCMS showed the desired mass was detected. The reaction mixture was diluted with water (20 mL) and then extracted with dichloromethane (15 mL×3). The combined organic layers were washed with brine (15 mL), dried over anhydrous sodium sulfate, filtered and concentrated under reduced pressure to give a residue. The residue was purified by column chromatography on silica gel (Petroleum ether/Ethyl acetate=100/1 to 5/1) to afford 7-(5-(bis(4-methoxybenzyl)amino)-3-methyl-2-(trifluoromethyl)phenyl)-2-(methylthio)-7,8-dihydro-5H-pyrano[4,3-d]pyrimidin-4-yl trifluoromethanesulfonate (1 g, 1.34 mmol, 63% yield) as a yellow solid. LCMS (ESI, m/z): 774.3 [M+H]⁺.

### Step 10: 5-(7-(5-(bis(4-methoxybenzyl)amino)-3-methyl-2-(trifluoromethyl)phenyl)-2-(methylthio)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide

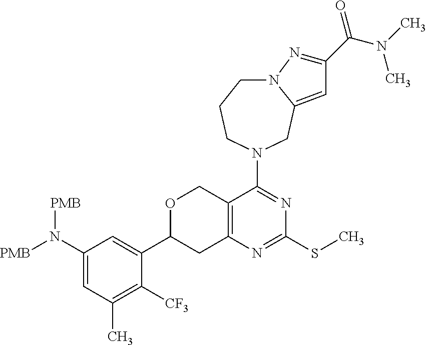

To a solution of N,N-dimethyl-5,6,7,8-tetrahydro-4H-pyrazolo[1,5-a][1,4]diazepine-2-carboxamide (420 mg, 2.02 mmol, 1.0 eq) in N,N-dimethylformamide (10 mL) was added N,N-diisopropylethylamine (1.30 g, 10.08 mmol, 5.0 eq) and 7-(5-(bis(4-methoxybenzyl)amino)-3-methyl-2-(trifluoromethyl)phenyl)-2-(methylthio)-7,8-dihydro-5H-pyrano[4,3-d]pyrimidin-4-yl trifluoromethanesulfonate (1.5 g, 2.02 mmol, 1.0 eq). The mixture was stirred at 50° C. for 1 h. LCMS showed the desired mass was detected. The reaction mixture was diluted with water (20 mL) and then extracted with ethyl acetate (20 mL×3). The combined organic layers were washed with brine (20 mL×2), dried over anhydrous sodium sulfate, filtered and concentrated under reduced pressure to give a residue. The residue was purified by column chromatography on silica gel (petroleum ether/ethyl acetate=1/1, then dichloromethane/methanol=10/1) to afford 5-(7-(5-(bis(4-methoxybenzyl)amino)-3-methyl-2-(trifluoromethyl)phenyl)-2-(methylthio)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (1.4 g, 1.75 mmol, 87% yield) as a yellow solid. LCMS (ESI, m/z): 802.3 [M+H]⁺.

### Step 11: 5-(7-(5-(bis(4-methoxybenzyl)amino)-3-methyl-2-(trifluoromethyl)phenyl)-2-(methylsulfinyl)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide

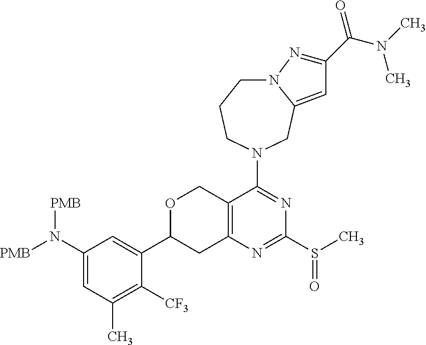

To a solution of 5-(7-(5-(bis(4-methoxybenzyl)amino)-3-methyl-2-(trifluoromethyl)phenyl)-2-(methylthio)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (700 mg, 0.87 mmol, 1.0 eq) in dichloromethane (10 mL) was added 3-chloro-benzenecarboperoxoic acid (177 mg, 0.87 mmol, 85% purity, 1.0 eq) at 0° C. The mixture was stirred at 0° C. for 0.5 h. LCMS showed the desired mass was detected. The reaction mixture was quenched by adding saturated aqueous sodium sulfite (50 mL) at 0° C., and then extracted with dichloromethane (20 mL×3). The combined organic layers were washed with saturated sodium sulfite solution (20 mL), brine (20 mL), dried over anhydrous sodium sulfate, filtered and concentrated under reduced pressure to afford 5-(7-(5-(bis(4-methoxybenzyl)amino)-3-methyl-2-(trifluoromethyl)phenyl)-2-(methylsulfinyl)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (700 mg, crude) as a yellow solid. LCMS (ESI, m/z): 818.3 [M+H]⁺.

### Step 12: 5-(7-(5-(bis(4-methoxybenzyl)amino)-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide

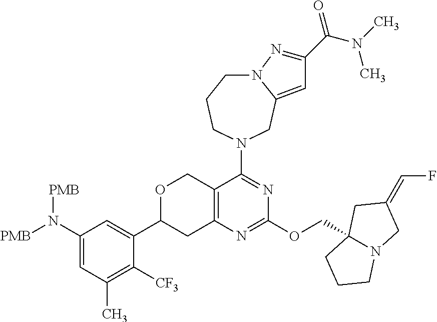

To a solution of 5-(7-(5-(bis(4-methoxybenzyl)amino)-3-methyl-2-(trifluoromethyl)phenyl)-2-(methylsulfinyl)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (700 mg, 0.86 mmol, 1.0 eq) and [(6Z,8S)-6-(fluoromethylene)-2,3,5,7-tetrahydro-1H-pyrrolizin-8-yl]methanol (213 mg, 1.03 mmol, 1.2 eq, hydrochloride) in tetrahydrofuran (10 mL) was added sodium tert-butoxide (329 mg, 3.42 mmol, 4.0 eq). The mixture was stirred at 20° C. for 0.5 h. LCMS showed the desired mass was detected. The reaction mixture was diluted with water (10 mL), and then extracted with ethyl acetate (10 mL×3). The combined organic layers were washed with brine (10 mL), dried over anhydrous sodium sulfate, filtered and concentrated under reduced pressure to give a residue. The residue was purified by prep-TLC (dichloromethane/methanol=10/1) to afford 5-(7-(5-(bis(4-methoxybenzyl)amino)-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (390 mg, 0.42 mmol, 49% yield) as a yellow solid. LCMS (ESI, m/z): 925.4 [M+H]⁺.

### Step 13: 5-(7-(5-amino-3-methyl-2-(trifluoromethyl)phenyl)-2-(((S,Z)-2-(fluoromethylene) tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a]1[1,4]diazepine-2-carboxamide

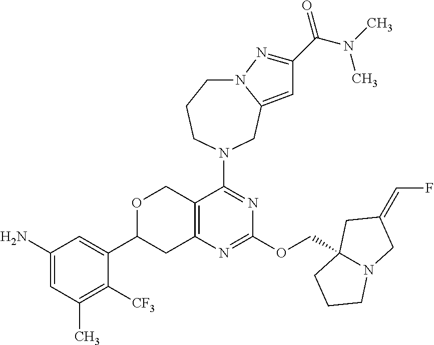 [SMILES: Cc1cc(N)cc(C2Cc3nc(OC[C@@]45CCCN4C/C(=C\F)C5)nc(N4CCCn5nc(C(=O)N(C)C)cc5C4)c3CO2)c1C(F)(F)F]

To a solution of 5-(7-(5-(bis(4-methoxybenzyl)amino)-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (390 mg, 0.42 mmol, 1.0 eq) in dichloromethane (1 mL) was added trifluoroacetic acid (3 mL). The mixture was stirred at 30° C. for 12 h. LCMS showed the desired mass was detected. The reaction mixture was concentrated under reduced pressure to give a residue. The residue was purified by prep-HPLC (column: YMC-Actus Triart C18 150×30 mm×7 um; mobile phase: [water(FA)-ACN]; gradient:20%-50% B over 10 min) to afford 5-(7-(5-amino-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (100 mg, 0.15 mmol, 35% yield) as a white solid. LCMS (ESI, m/z): 685.3 [M+H]⁺, ¹H NMR (400 MHz, DMSO-d₆) δ: 6.84-6.55 (m, 3H), 6.41 (s, 1H), 5.76 (s, 2H), 5.05-4.93 (m, 2H), 4.74-4.63 (m, 3H), 4.50-4.40 (m, 2H), 3.92-3.79 (m, 4H), 3.69-3.63 (m, 1H), 3.29 (s, 1H), 3.24 (s, 3H), 3.00-2.96 (m, 1H), 2.93 (s, 3H), 2.84-2.77 (m, 1H), 2.68-2.60 (m, 1H), 2.34-2.26 (m, 4H), 2.26-2.06 (m, 2H), 2.03-1.59 (m, 6H).

### Step 14: 5-((S)-7-(5-amino-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (Example 302)

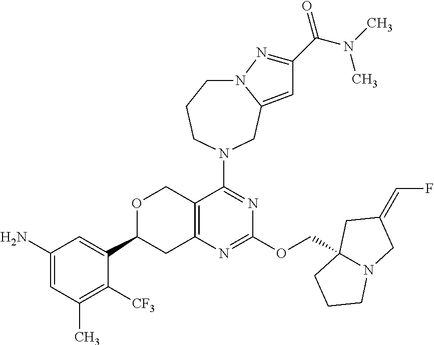 [SMILES: Cc1cc(N)cc([C@@H]2Cc3nc(OC[C@@]45CCCN4C/C(=C\F)C5)nc(N4CCCn5nc(C(=O)N(C)C)cc5C4)c3CO2)c1C(F)(F)F]

The mixture of diastereomers 5-(7-(5-amino-3-methyl-2-(trifluoromethyl)phenyl)-2-(((S,Z)-2-(fluoromethylene)tetrahydro-1H-pyrrolizin-7a(5H)-yl)methoxy)-7, 8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (50 mg, 0.07 mmol, 1.0 eq) was separated by SFC (column: DAICEL CHIRALPAK AD (250 mm*30 mm,10 um); mobile phase: [CO2-i-PrOH(0.1% NH₃H2O)]; B %:45%, isocratic elution mode). The second eluent was identified as the diastereomer 5-((S)-7-(5-amino-3-methyl-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide (12.52 mg, 0.02 mmol, 25% yield, 97.89% purity, t_{R}=1.903 min) as a yellow solid. LCMS (ESI, m/z): 685.3 [M+H]⁺, ¹H NMR (400 MHz, DMSO-d₆) δ: 6.90-6.64 (m, 1H), 6.57 (s, 1H), 6.41 (s, 1H), 5.76 (s, 2H), 5.06-4.92 (m, 2H), 4.78-4.65 (m, 3H), 4.62-4.37 (m, 3H), 3.99-3.80 (m, 4H), 3.52-3.39 (m, 3H), 3.24 (s, 3H), 2.93 (s, 3H), 2.84-2.78 (m, 1H), 2.68-2.64 (m, 1H), 2.63-2.60 (m, 1H), 2.32-2.28 (m, 3H), 2.20-1.67 (m, 8H).

### Synthesis of 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide (Example 450)

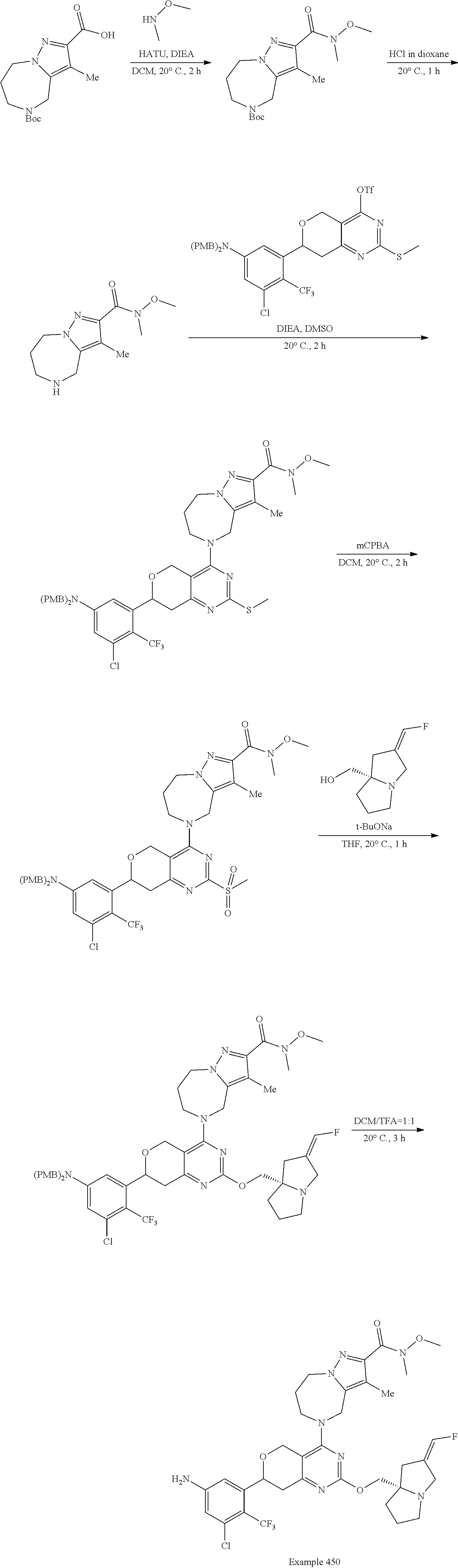

### Step 1: Synthesis of tert-butyl 2-(methoxy(methyl)carbamoyl)-3-methyl-7,8-dihydro-4H-pyrazolo[1,5-a][1,4]diazepine-5(6H)-carboxylate

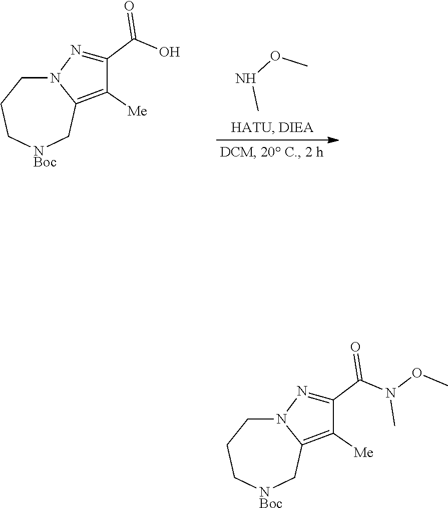

To the mixture of 5-[(tert-butoxy)carbonyl]-3-methyl-4H,6H,7H,8H-pyrazolo[1,5-a][1,4]diazepine-2-carboxylic acid (300 mg, 1.01 mmol, 1 eq, the procedure for 155-BQ-1, please see the procedure of Erasca-155-BQ-P1, P2) in DCM (10 mL) was added N,O-Dimethylhydroxylamine Hydrochloride (198.1 mg, 2.03 mmol, 2 eq), DIEA (525.1 mg, 4.06 mmol, 4 eq) and HATU (579.4 mg, 1.5 mmol, 1.5 eq). The mixture was stirred at 23° C. for 1 h. The mixture was diluted with H₂O, extracted with DCM (10 mL×3). The combined organic layer was washed with brine (50 mL), dried over Na₂SO₄, filtered then concentrated in vacuo to give a residue. Then the residue was purified by column chromatography (PE/EA=10/1 to 5/1) to give tert-butyl 2-(methoxy(methyl)carbamoyl)-3-methyl-7, 8-dihydro-4H-pyrazolo[1,5-a][1,4]diazepine-5(6H)-carboxylate (270 mg, 79.1% yield) as yellow solid. ESI-MS m/z=339.2[M+1]+. Calculated MW: 338.2

### Step 2: Synthesis of N-methoxy-N,3-dimethyl-5,6,7,8-tetrahydro-4H-pyrazolo[1,5-a][1,4]diazepine-2-carboxamide

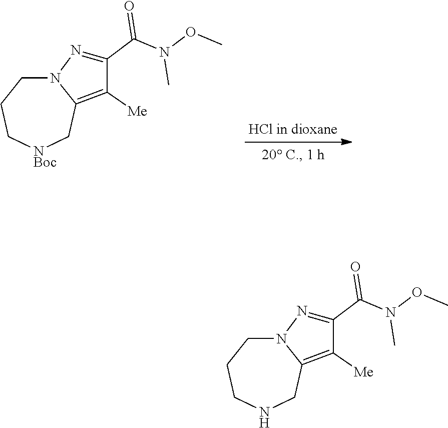 [SMILES: CON(C)C(=O)c1nn2c(c1C)CN(C(=O)OC(C)(C)C)CCC2 ; CON(C)C(=O)c1nn2c(c1C)CNCCC2]

A mixture of tert-butyl 2-[methoxy(methyl)carbamoyl]-3-methyl-4H,6H,7H,8H-pyrazolo[1,5-a][1,4]diazepine-5-carboxylate (270 mg, 0.8 mmol, 1.0 eq.) and HCl in dioxane (2 ml, 1.6 mmol, 2 eq) in 1,4-dioxane (2 mL) was stirred at 23° C. for 1 h. The reaction was concentrated under vacuum to give N-methoxy-N,3-dimethyl-5,6,7,8-tetrahydro-4H-pyrazolo[1,5-a][1,4]diazepine-2-carboxamide. (180 mg, 94.8% yield) as white solid, which was used directly without further purification. ESI-MS m/z=239.2 [M+1]*. Calculated MW: 238.2.

### Step 3: Synthesis of 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-N-methoxy-N,3-dimethyl-5,6,7,8-tetrahydro-4H-pyrazolo[1,5-a]1[1,4]diazepine-2-carboxamide

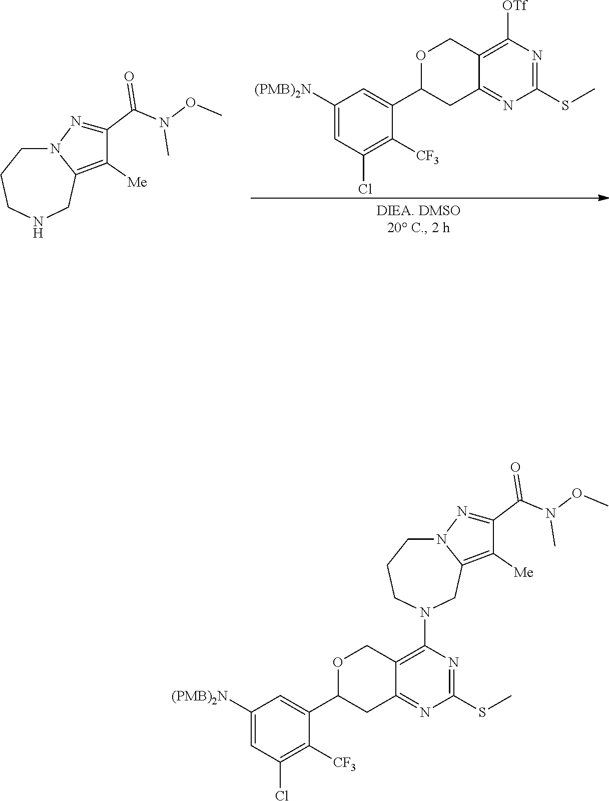

To the mixture of 7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl trifluoromethanesulfonate (200 mg, 0.26 mmol, 1 eq) in DMSO (6 mL) was added N-methoxy-N,3-dimethyl-4H,5H,6H,7H,8H-pyrazolo[1,5-a][1,4]diazepine-2-carboxamide (124.7 mg, 0.52 mmol, 2 eq) and DIEA (135.3 mg, 1.05 mmol, 4 eq). The mixture was stirred at 23° C. for 1 h. The mixture was diluted with H₂O, extracted with DCM (10 mL×3). The combined organic layer was washed with brine (10 mL), dried over Na₂SO₄, filtered then concentrated in vacuo to give a residue. Then the residue was purified by column chromatography (PE/EA=5/1 to 1/1) to give 5-(7-(5-(bis(4-methoxybenzyl)amino)-3-chloro-2-(trifluoromethyl)phenyl)-2-(methylthio)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide (210 mg, 94.9% yield) as yellow solid. ESI-MS m/z=852.2 [M+1]⁺. Calculated MW: 851.2.

### Step 4: Synthesis of 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide

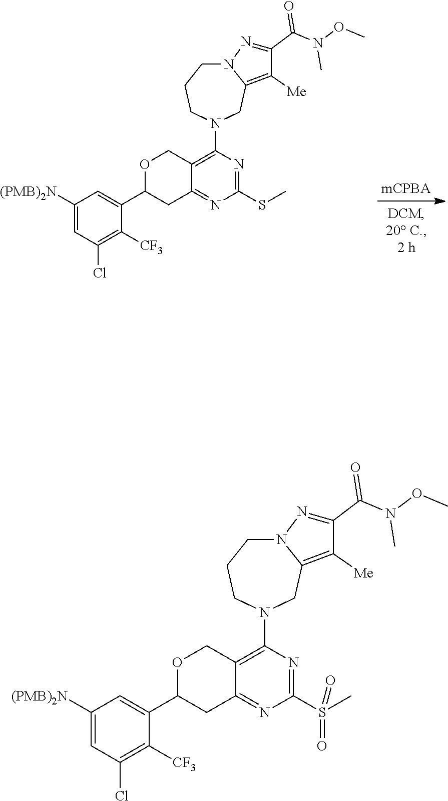

A mixture of 5-[7-(5-{bis[(4-methoxyphenyl)methyl]aminoI-3-chloro-2-(trifluoromethyl)phenyl)-2-(methylsulfanyl)-5H,7H,8H-pyrano[4,3-d]pyrimidin-4-yl]-N-methoxy-N,3-dimethyl-4H,6H,7H,8H-pyrazolo[1,5-a][1,4]diazepine-2-carboxamide (210 mg, 0.25 mmol, 1.0 eq.) and mCPBA (127.3 mg, 0.75 mmol, 3 eq) in DCM (4 mL) was stirred at 23° C. for 1 h. The reaction was diluted with water (30 mL) and the mixture was neutralized with aqueous solution of Na₂₀₃S2 and extracted 3 times with EA. Then the residue was purified by column chromatography (DCM/MeOH=10/1) to give 5-(7-(5-(bis(4-methoxybenzyl)amino)-3-chloro-2-(trifluoromethyl)phenyl)-2-(methylsulfonyl)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide (210 mg, 95.1% yield) as yellow solid. ESI-MS m/z=884.2 [M+1]⁺. Calculated MW: 883.2

### Step 5: Synthesis of 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide

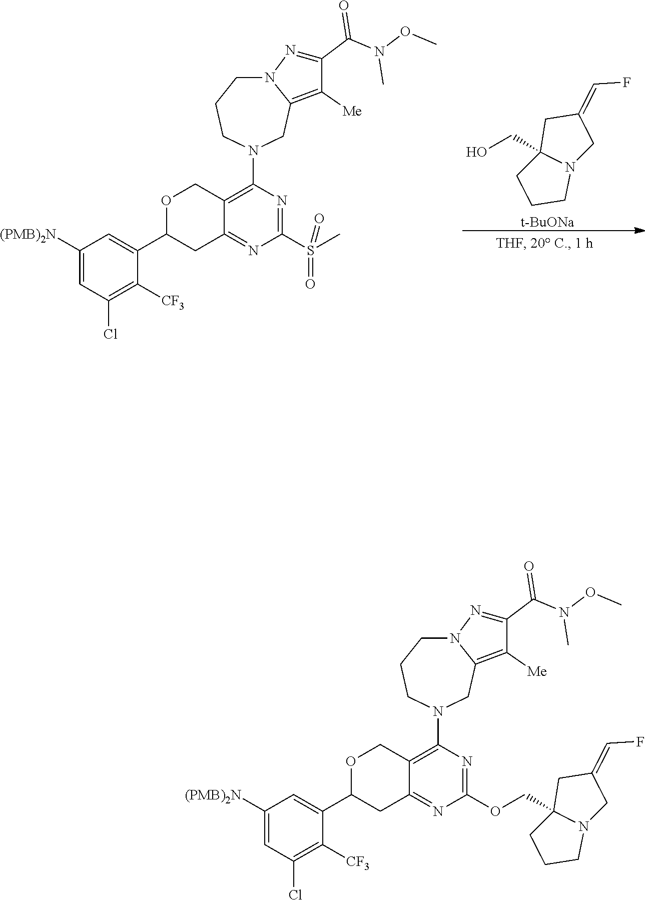

To the mixture of 5-[7-(5-{bis[(4-methoxyphenyl)methyl]amino}-3-chloro-2-(trifluoromethyl)phenyl)-2-methanesulfonyl-5H,7H,8H-pyrano[4,3-d]pyrimidin-4-yl]-N-methoxy-N,3-dimethyl-4H,6H,7H,8H-pyrazolo[1,5-a][1,4]diazepine-2-carboxamide (150 mg, 0.17 mmol, 1 eq) in THF (4 mL) was added [(2Z,7aS)-2-(fluoromethylidene)-tetrahydro-1H-pyrrolizin-7a-yl]methanol (43.6 mg, 0.25 mmol, 1.5 eq) and sodium tert-butoxide (32.6 mg, 0.34 mmol, 2 eq). The mixture was stirred at 23° C. for 1 h. The mixture was diluted with H₂O, extracted with DCM (10 mL×3). The combined organic layer was washed with brine (10 mL), dried over Na₂SO₄, filtered then concentrated in vacuo to give a residue. Then the residue was purified by column chromatography (DCM/MeOH=10/1) to give 5-(7-(5-(bis(4-methoxybenzyl)amino)-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide (100 mg, 60.4% yield) as yellow solid. ESI-MS m/z=975.2 [M+1]⁺. Calculated MW: 974.2.

### Step 6: Synthesis of 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide

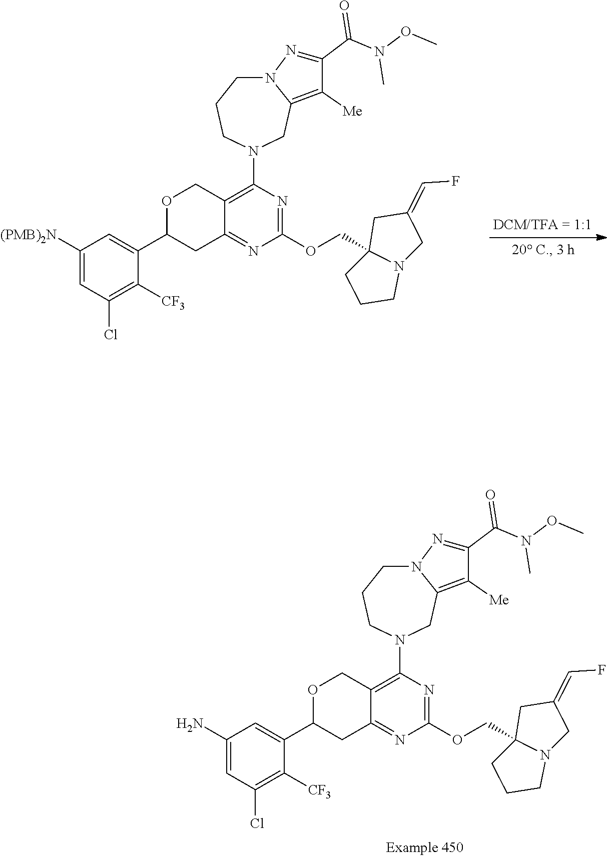

A mixture of 5-(2-{[(2Z,7aS)-2-(fluoromethylidene)-tetrahydro-1H-pyrrolizin-7a-yl]methoxy}-7-(5-{bis[(4-methoxyphenyl)methyl]amino}-3-chloro-2-(trifluoromethyl)phenyl)-5H,7H,8H-pyrano[4,3-d]pyrimidin-4-yl)-N-methoxy-N,3-dimethyl-4H,6H,7H,8H-pyrazolo[1,5-a][1,4]diazepine-2-carboxamide (100 mg, 0.1 mmol, 1.0 eq.) in DCM:TFA=1:1 (4 mL) was stirred at 23° C. for 1 h. The reaction was concentrated in vacuo to give a residue. Then the residue was purified by HPLC(H₂O (0.1% TFA)/ACN=80%/20% to 70%/20%) to give 5-(7-(5-amino-3-chloro-2-(trifluoromethyl)phenyl)-2-(((S,Z)-2-(fluoromethylene)tetrahydro-1H-pyrrolizin-7a(5H)-yl)methoxy)-7,8-dihydro-5H-pyrano[4,3-d]pyrimidin-4-yl)-N-methoxy-N,3-dimethyl-5,6,7,8-tetrahydro-4H-pyrazolo[1,5-a][1,4]diazepine-2-carboxamide (31 mg, 4.2% yield) as white solid. ESI-MS m/z=735.2[M+1]⁺. Calculated MW: 734.2. ¹H NMR (400 MHz, MeOD) δ 6.99 (s, 0.5H), 6.96 (d, J=2.1 Hz, 1H), 6.77 (d, J=11.5 Hz, 0.5H), 6.73 (d, J=2.2 Hz, 1H), 5.15 (d, J=8.2 Hz, 1H), 5.05 (d, J=13.7 Hz, 1H), 4.82 (d, J=4.2 Hz, 4H), 4.50 (d, J=21.0 Hz, 2H), 4.33 (m, 2H), 4.12 (d, J=14.8 Hz, 1H), 3.97 (d, J=14.0 Hz, 1H), 3.85 (d, J=8.0 Hz, 1H), 3.74 (d, J=18.1 Hz, 4H), 3.40 (s, 3H), 3.27 (d, J=10.8 Hz, 1H), 2.96 (dd, J=51.2, 17.1 Hz, 2H), 2.77 (t, J=12.8 Hz, 2H), 2.27 (m, 6H), 2.14 (dd, J=19.1, 12.1 Hz, 3H).

### Synthesis of 5-(7-(3-amino-2-fluoro-5-methyl-6-(trifluoromethyl)phenyl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-chloro-N-methoxy-N-methyl-5,6,7,8-tetrahydro-4H-pyrazolo[1,5-a][1,4]diazepine-2-carboxamide (Example 464)

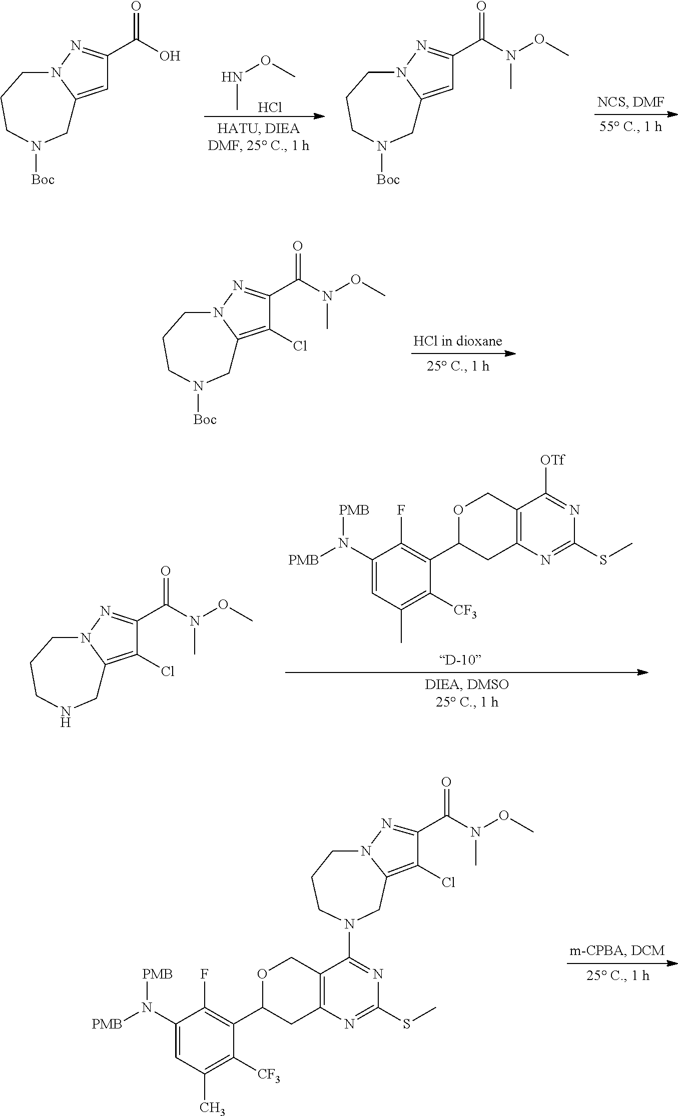

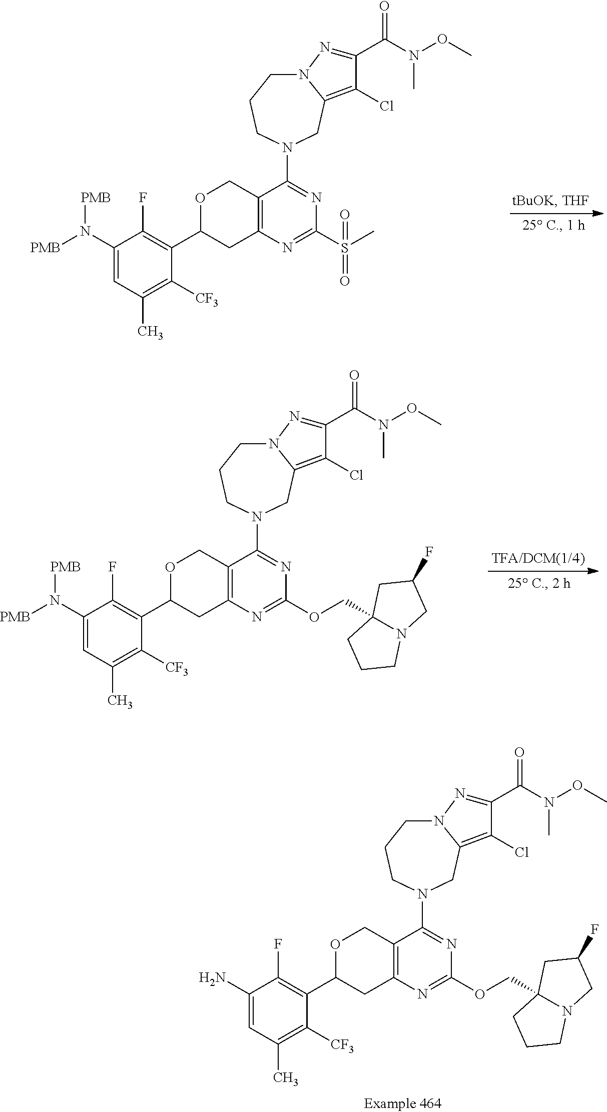

### Step 1: Synthesis of tert-butyl 2-[methoxy(methyl)carbamoyl]-4H,6H,7H,8H-pyrazolo[1,5-a][1,4]diazepine-5-carboxylate

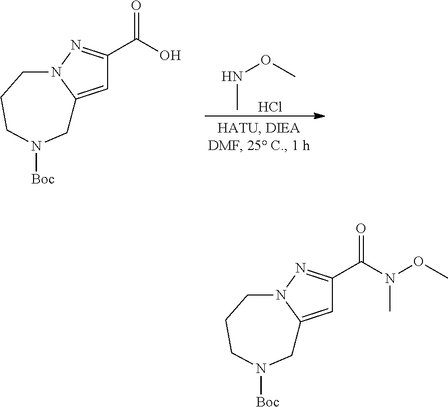

To a mixture of 5-[(tert-butoxy)carbonyl]-4H,6H,7H,8H-pyrazolo[1,5-a][1,4]diazepine-2-carboxylic acid (300 mg, 1.0 mmol, 1.0 eq, the procedure for 154-B-8, please see the procedure of Erasca-154-B-11) and N,O-Dimethylhydroxylamine hydrochloride (208 mg, 2.1 mmol, 2.0 eq, cas; 6638-79-5) in DMF (10 mL) was added DIEA (413 mg, 3.2 mmol, 3.0 eq) and HATU (608 mg, 1.6 mmol, 1.5 eq) at 25° C. The mixture was stirred at 25° C. for lh. After completion, the mixture was quenched with brine (10 mL), extracted with EtOAc (15 mL*3). The organic layer was washed with brine (8 mL*3), dried over anhydrous Na₂SO₄, filtrated and concentrated in vacuo to give a residue. The residue was purified by SGC (UV254, DCM:MeOH=20:1) to afford tert-butyl 2-[methoxy(methyl)carbamoyl]-4H,6H,7H,8H-pyrazolo[1,5-a]1[1,4]diazepine-5-carboxylate (300 mg, yield:78%) as a yellow oil. MS (ESI, pos. ion) m/z: 325.3. (M+Na). Calculated MW: 324.38.

### Step 2: Synthesis of tert-butyl 3-chloro-2-[methoxy(methyl)carbamoyl]-4H,6H,7H,8H-pyrazolo[1,5-a][1,4]diazepine-5-carboxylate

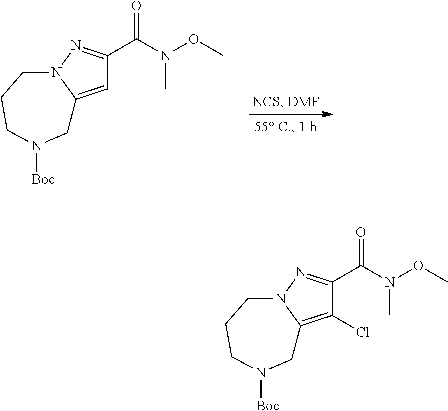 [SMILES: CON(C)C(=O)c1cc2n(n1)CCCN(C(=O)OC(C)(C)C)C2 ; CON(C)C(=O)c1nn2c(c1Cl)CN(C(=O)OC(C)(C)C)CCC2]

A mixture of tert-butyl 2-[methoxy(methyl)carbamoyl]-4H,6H,7H,8H-pyrazolo[1,5-a][1,4]diazepine-5-carboxylate (300 mg, 0.92 mmol, 1.0 eq) and NCS (148 mg, 1.1 mmol, 1.2 eq) in DMF (10 mL) was stirred at 55° C. for 1 h. After completion, the mixture was quenched with brine (10 mL), extracted with EtOAc (15 mL*3). The organic layer was washed with brine (8 mL*3), dried over anhydrous Na₂SO₄, filtrated and concentrated in vacuo to give a residue. The residue was purified by SGC (UV254, DCM:MeOH=20:1) to afford tert-butyl 3-chloro-2-[methoxy(methyl)carbamoyl]-4H,6H,7H,8H-pyrazolo[1,5-a][1,4]diazepine-5-carboxylate (300 mg, yield:81%) as a yellow oil. MS (ESI, pos. ion) m/z: 359.1. (M+1). Calculated MW: 358.82.

### Step 3: Synthesis of 3-chloro-N-methoxy-N-methyl-4H,5H,6H,7H,8H-pyrazolo[1,5-a][1,4]diazepine-2-carboxamide

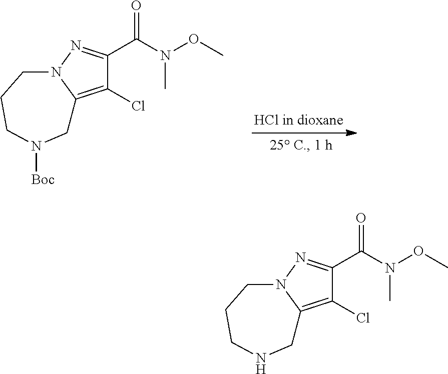 [SMILES: CON(C)C(=O)c1nn2c(c1Cl)CN(C(=O)OC(C)(C)C)CCC2 ; CON(C)C(=O)c1nn2c(c1Cl)CNCCC2]

A mixture of tert-butyl 3-chloro-2-[methoxy(methyl)carbamoyl]-4H,6H,7H,8H-pyrazolo[1,5-a][1,4]diazepine-5-carboxylate (300 mg, 0.84 mmol, 1.0 eq) in HCl in dioxane (2M) (10 mL) was stirred at 25° C. for 1 h. After completion, the mixture was concentrated in vacuo to afford 3-chloro-N-methoxy-N-methyl-4H,5H,6H,7H,8H-pyrazolo[1,5-a][1,4]diazepine-2-carboxamide (230 mg, yield:96%) as a colorless oil. MS (ESI, pos. ion) m/z: 259.1. (M+1). Calculated MW: 258.71.

### Step 4: Synthesis of 5-(7-{3-[bis(2-methoxy-5-methylphenyl)amino]-2-fluoro-5-methyl-6-(trifluoromethyl)phenyl}-2-(methylsulfanyl)-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide

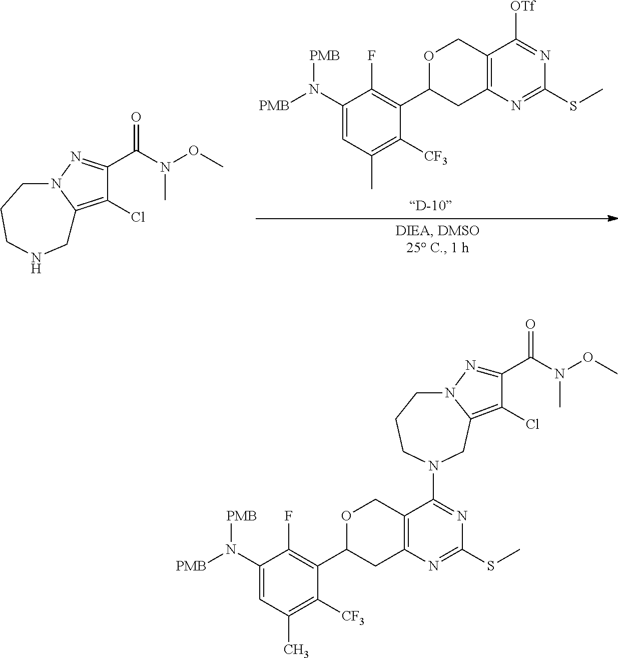

To a mixture of 3-chloro-N-methoxy-N-methyl-4H,5H,6H,7H,8H-pyrazolo[1,5-a][1,4]diazepine-2-carboxamide (100 mg, 0.39 mmol, 1.0 eq) and 7-(3-(bis(4-methoxybenzyl)amino)-2-fluoro-5-methyl-6-(trifluoromethyl)phenyl)-2-(methylthio)-7,8-dihydro-5H-pyrano[4,3-d]pyrimidin-4-yl trifluoromethanesulfonate (147 mg, 0.19 mmol, 0.5 eq, for the procedure of 155-D-10, please see the report of Erasca-155-D) in DMSO (8 mL) was added DIEA (150 mg, 1.2 mmol, 3.0 eq) at 25° C. The mixture was stirred at 25° C. for 1 h. After completion, the mixture was quenched with brine (10 mL), extracted with EtOAc (15 mL*3). The organic layer was washed with brine, dried over anhydrous Na₂SO₄, filtrated and concentrated in vacuo to give a residue. The residue was purified by SGC (UV254, DCM:MeOH=20:1) to afford 5-(7-{3-[bis(2-methoxy-5-methylphenyl)amino]-2-fluoro-5-methyl-6-(trifluoromethyl)phenyl}-2-(methylsulfanyl)-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (100 mg, yield:28%) as a yellow solid. MS (ESI, pos. ion) m/z: 870.2. (M+1). Calculated MW: 870.36.

### Step 5: Synthesis of 5-(7-{3-[bis(2-methoxy-5-methylphenyl)amino]-2-fluoro-5-methyl-6-(trifluoromethyl)phenyl}-2-methanesulfonyl-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide

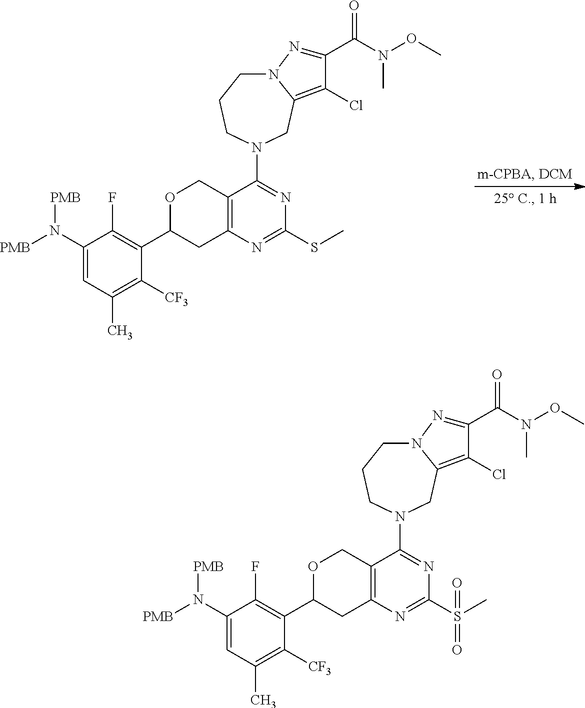

To a mixture of 5-(7-{3-[bis(2-methoxy-5-methylphenyl)amino]-2-fluoro-5-methyl-6-(trifluoromethyl)phenyl}-2-(methylsulfanyl)-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (90 mg, 0.10 mmol, 1.0 eq) in DCM (10 mL) was added m-CPBA (54 mg, 0.31 mmol, 1.5 eq) at 25° C. The mixture was stirred at 25° C. for 1 h. After completion, the mixture was quenched with a. q. Na₂S204, extracted with DCM (15 mL*3). The organic layer was washed with a. q. NaHCO₃ and brine, dried over anhydrous Na₂SO₄, filtrated and concentrated in vacuo to give a residue. The residue was purified by SGC (UV254, DCM:MeOH=20:1) to afford 5-(7-{3-[bis(2-methoxy-5-methylphenyl)amino]-2-fluoro-5-methyl-6-(trifluoromethyl)phenyl}-2-methanesulfonyl-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (90 mg, yield:87%) as a yellow oil. MS (ESI, pos. ion) m/z: 902.1. (M+1). Calculated MW: 902.36.

### Step 6: Synthesis of 5-(2-{[(2R,7aS)-2-fluoro-hexahydropyrrolizin-7a-yl]methoxy}-7-{3-[bis(2-methoxy-5-methylphenyl)amino]-2-fluoro-5-methyl-6-(trifluoromethyl)phenyl}-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide

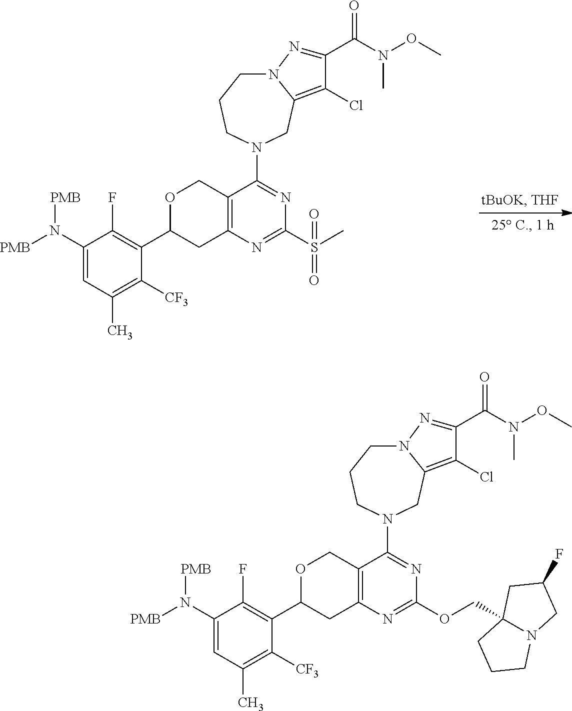

To a mixture of [(2R,7aS)-2-fluoro-hexahydropyrrolizin-7a-yl]methanol (53 mg, 0.33 mmol, 1.0 eq) in THF (10 mL) was added NaO^{t}Bu (32 mg, 0.33 mmol, 3.0 eq) and 5-(7-{3-[bis(2-methoxy-5-methylphenyl)amino]-2-fluoro-5-methyl-6-(trifluoromethyl)phenyl}-2-methanesulfonyl-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (100 mg, 0.11 mmol, 3.0 eq) at 0° C. The mixture was stirred at 25° C. for 1 h. After completion, the mixture was quenched with H₂O (10 mL), extracted with EA (15 mL*3). The organic layer was washed with brine, dried over anhydrous Na₂SO₄, filtrated and concentrated in vacuo to give a residue. The residue was purified by SGC (UV254, EA:MeOH=20:1) to afford 5-(2-{[(2R,7aS)-2-fluoro-hexahydropyrrolizin-7a-yl]methoxy}-7-{3-[bis(2-methoxy-5-methylphenyl)amino]-2-fluoro-5-methyl-6-(trifluoromethyl)phenyl}-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (90 mg, yield:75%) as a yellow oil. MS (ESI, pos. ion) m/z: 981.1. (M+1). Calculated MW: 981.46.

### Step 7: Synthesis of 5-(2-{[(2R,7aS)-2-fluoro-hexahydropyrrolizin-7a-yl]methoxy}-7-[3-amino-2-fluoro-5-methyl-6-(trifluoromethyl)phenyl]-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide

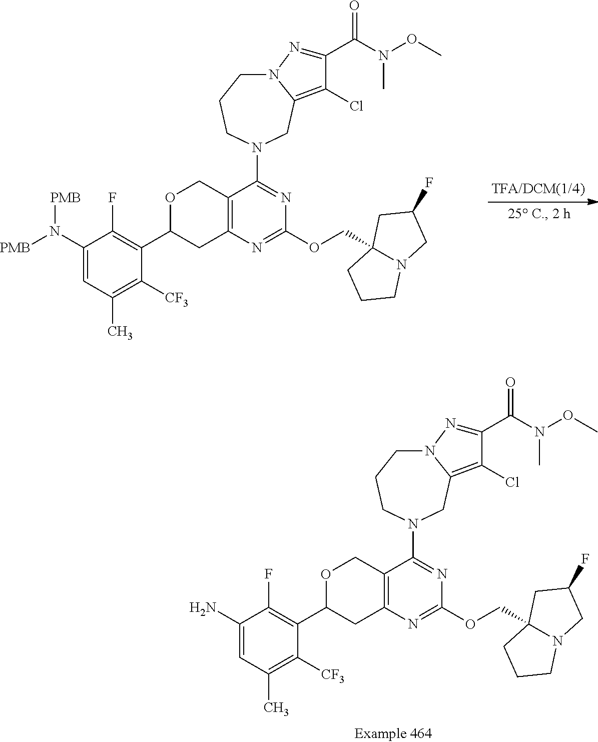

A mixture of 5-(2-{[(2R,7aS)-2-fluoro-hexahydropyrrolizin-7a-yl]methoxy}-7-{3-[bis(2-methoxy-5-methylphenyl)amino]-2-fluoro-5-methyl-6-(trifluoromethyl)phenyl}-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (60 mg, 0.061 mmol, 1.0 eq) in TFA/DCM=1/4 (5 mL) was stirred at 25° C. for 2 h. After completion, the mixture concentrated in vacuo to give a residue. The residue was purified by reverse phase (c-18, CH₃CN/H₂O (0.1% TFA)=40%) to afford 5-(2-{[(2R,7aS)-2-fluoro-hexahydropyrrolizin-7a-yl]methoxy}-7-[3-amino-2-fluoro-5-methyl-6-(trifluoromethyl)phenyl]-5H,7H,8H-pyrano[4,3-d]pyrimidin-4-yl)-3-chloro-N-methoxy-N-methyl-4H,6H,7H,8H-pyrazolo[1,5-a][1,4]diazepine-2-carboxamide (30 mg, yield:66%) as a white solid. MS (ESI, pos. ion) m/z: 741.1. (M+1). Calculated MW: 741.16. ¹H NMR (400 MHz, MeOD) δ 6.70 (d, J=8.5 Hz, 1H), 5.63 (s, 0.5H), 5.50 (s, 0.5H), 5.22 (d, J=6.9 Hz, 1H), 4.98 (t, J=13.4 Hz, 2H), 4.77 (d, J=13.7 Hz, 2H), 4.61 (d, J=17.3 Hz, 1H), 4.53-4.45 (m, 2H), 4.21 (d, J=9.3 Hz, 2H), 4.01-3.89 (m, 2H), 3.85 (d, J=14.4 Hz, 3H), 3.75 (s, 3H), 3.45-3.36 (m, 4H), 2.89 (d, J=17.1 Hz, 1H), 2.74-2.52 (m, 2H), 2.46-2.28 (m, 7H), 2.17 (s, 2H).

### Synthetic Schemes of Vinyl “Tail”

### (2-(2,2-difluorovinyl)tetrahydro-1H-pyrrolizin-7a(5H)-yl)methanol (5-1) and ((2R,7aR)-2-(2,2-difluorovinyl)tetrahydro-1H-pyrrolizin-7a(5H)-yl)methanol

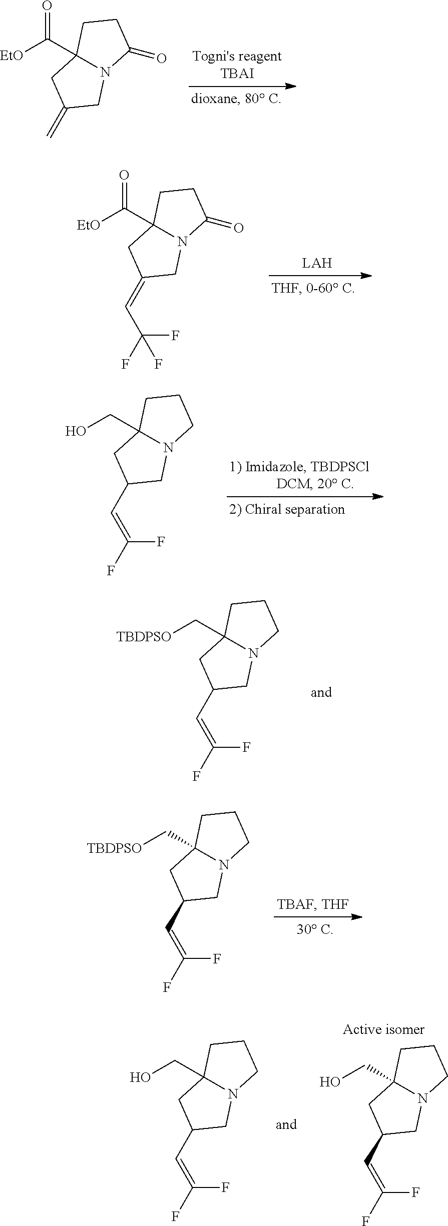

### Step 1: Synthesis of ethyl 5-oxo-2-(2,2,2-trifluoroethylidene)tetrahydro-1H-pyrrolizine-7a(5H)-carboxylate

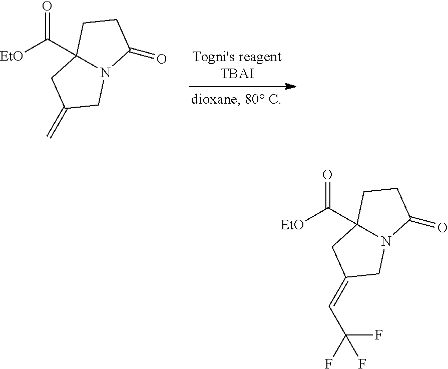 [SMILES: C=C1CN2C(=O)CCC2(C(=O)OCC)C1 ; CCOC(=O)C12CCC(=O)N1C/C(=C\C(F)(F)F)C2]

A mixture of ethyl 2-methylidene-5-oxo-tetrahydropyrrolizine-7a-carboxylate (1, 1.3 g, 6.2 mmol), 1-trifluoroMethyl-1,2-benziodoxol-3(1H)-one (3.9 g, 0.012 mol) and TBAI (1.2 g, 0.0031 mol) in dioxane (20 mL) was stirred at 80° C. for 10 h under N₂. After completion, the mixture was quenched with H₂O (20 mL), extracted with EtOAc (30 mL*3). The organic layer was washed with brine, dried over anhydrous Na₂SO₄, filtrated and concentrated in vacuo to give a residue. The residue was purified by SGC (UV254, Petroleum ether:EtOAc=4:1) to afford ethyl 5-oxo-2-(2,2,2-trifluoroethylidene)tetrahydro-1H-pyrrolizine-7a(5H)-carboxylate as a yellow oil (1.6 g, yield: 84%). LCMS (ESI) m/z=278.2. [M+H]⁺. Calculated MS: 277.24.

### Step 2: synthesis of 2-fluoro-N,N-bis(4-methoxybenzyl)-5-(2-(2,2-difluorovinyl)tetrahydro-1H-pyrrolizin-7a(5H)-yl)methanol

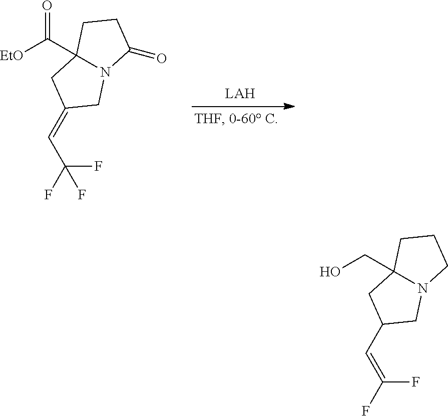 [SMILES: CCOC(=O)C12CCC(=O)N1C/C(=C\C(F)(F)F)C2 ; OCC12CCCN1CC(C=C(F)F)C2]

To a mixture of ethyl 5-oxo-2-(2,2,2-trifluoroethylidene)tetrahydro-1H-pyrrolizine-7a(5H)-carboxylate (2, 1.5 g, 5.4 mmol) in THF (60 mL) was added LiAlH₄ (16 mL, 16 mmol, 1M in THF) at 0° C. The mixture was stirred at 60° C. for 3 h. After completion, the mixture was quenched with Na₂SO₄·10H₂O, filtrated and concentrated in vacuo to afford (2-(2,2-difluorovinyl)tetrahydro-1H-pyrrolizin-7a(5H)-yl)methanol as a yellow oil (1.1 g, yield: 91%). LCMS (ESI) m/z=204.2. [M+H]⁺. Calculated MS: 203.23.

### Step 3: synthesis of 7a-(((tert-butyldiphenylsilyl)oxy)methyl)-2-(2,2-difluorovinyl)hexahydro-1H-pyrrolizine (4-1) and (2R,7aR)-7a-(((tert-butyldiphenylsilyl)oxy)methyl)-2-(2,2-difluorovinyl)hexahydro-1H-pyrrolizine

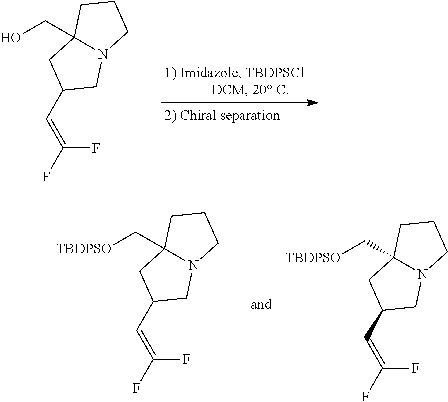

To a mixture of (2-(2,2-difluorovinyl)tetrahydro-1H-pyrrolizin-7a(5H)-yl)methanol (3, 1.1 g, 5.4 mmol) and imidazole (1.1 g, 16 mmol) in dioxane (30 mL) was added TBDPSCl (3.0 g, 11 mmol) at 20° C. The mixture was stirred at 20° C. for 16 h. After completion, the mixture was quenched with H₂O (15 mL), extracted with EtOAc (20 mL*3). The organic layer was washed with brine, dried over anhydrous Na₂SO₄, filtrated, and concentrated in vacuo to give a residue. The residue was purified by SGC (UV254, Petroleum ether:EtOAc=10:1) to afford crude product as a yellow oil. MS (ESI, pos. ion) m/z: 442.2. (M+1). Calculated MW: 441.64. The product was separated by SFC (Daicel CHIRALPAK IC_3, 3.0*150 mm, 3 um, CO₂/IPA (0.1% DEA)=90/10) to afford 7a-(((tert-butyldiphenylsilyl)oxy)methyl)-2-(2,2-difluorovinyl)hexahydro-1H-pyrrolizine (4-1) (0.68 g, RT=0.980) and (2R,7aR)-7a-(((tert-butyldiphenylsilyl)oxy)methyl)-2-(2,2-difluorovinyl)hexahydro-1H-pyrrolizine (0.7 g, RT=1.254) as yellow oil. Absolute configuration of product was determined as (R,R) by X-ray crystallography of Compound 447.

### Step 4: synthesis of [2-(2,2-difluoroethenyl)-hexahydropyrrolizin-7a-yl]methanol

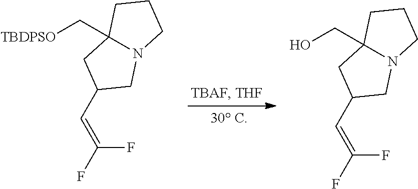

To a mixture of 7a-{[(tert-butyldiphenylsilyl)oxy]methyl}-2-(2,2-difluoroethenyl)-hexahydropyrrolizine (4-1, 150 mg, 0.34 mmol) in THF (10 mL) was added TBAF (0.68 mL, 0.68 mmol, 1M in THF) at 25° C. The mixture was stirred at 30° C. for 16 h. After completion, the mixture was concentrated in vacuo to afford [2-(2,2-difluoroethenyl)-hexahydropyrrolizin-7a-yl]methanol as a yellow oil (140 mg, crude). MS (ESI, pos. ion) m/z: 204.1. (M+1). Calculated MW: 203.23.

### Step 4: synthesis of ((2R,7aR)-2-(2,2-difluorovinyl)tetrahydro-1H-pyrrolizin-7a(5H)-yl)methanol

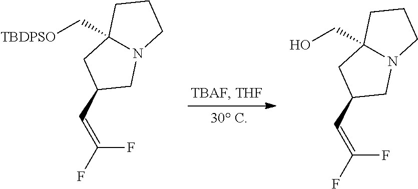

To a mixture of (2R,7aR)-7a-(((tert-butyldiphenylsilyl)oxy)methyl)-2-(2,2-difluorovinyl)hexahydro-1H-pyrrolizine (4-2, 150 mg, 0.34 mmol) in THF (10 mL) was added TBAF (0.68 mL, 0.68 mmol, 1M in THF) at 25° C. The mixture was stirred at 30° C. for 16 h. After completion, the mixture was concentrated in vacuo to give a residue. The residue was purified by reverse phase (c-18, CH₃CN/H₂O+(0.1% FA)=3%) to afford ((2R,7aR)-2-(2,2-difluorovinyl)tetrahydro-1H-pyrrolizin-7a(5H)-yl)methanol as a yellow oil (70 mg, yield: 91%). MS (ESI, pos. ion) m/z: 204.1. (M+1). Calculated MW: 203.23.

The pERK IC₅₀ activities of exemplary compounds of the present application are provided in Tables 1a and 1b below.

TABLE 1a

| | | PERK | | | |
|---|---|---|---|---|---|
| | | IC₅₀ | | | |
| | | (AsPC- | | | |
| | | 1, 4H) | | MS | |
| Ex. | Structure | nM | * | (ESI) | 1H-NMR |
| 201 | 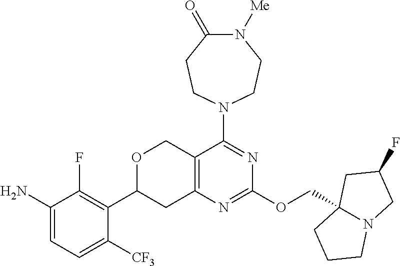 [SMILES: CN1CCN(c2nc(OC[C@@]34CCCN3C[C@H](F)C4)nc3c2COC(c2c(C(F)(F)F)ccc(N)c2F)C3)CCC1=O] | >10,000 | D | 597.2 | ¹H NMR (400 MHz, CDCl₃) δ 7.30 (s, 1H) 6.78 (t, J = 8.4 Hz, 1H) 5.09-5.37 (m, 2H) 4.63-4.81 (m, 2H) 4.06-4.17 (m, 3H) 3.92-4.05 (m, 1H) 3.58-3.66 (m, 3H) 3.49-3.58 (m, 2H) 3.15-3.46 (m, 5H) 3.04 (s, 3H) 2.94- 3.01 (m, 2H) 2.83- 2.92 (m, 1H) 2.71- 2.80 (m, 1H) 2.09- 2.33 (m, 3H) 1.79- 2.01 (m, 3H) |
| 202 | 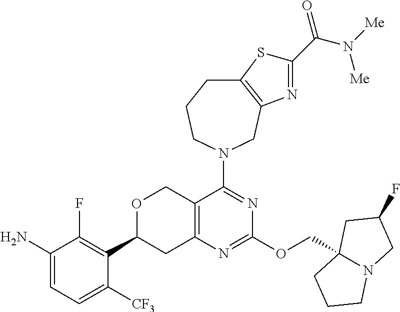 [SMILES: CN(C)C(=O)c1nc2c(s1)CCCN(c1nc(OC[C@@]34CCCN3C[C@H](F)C4)nc3c1CO[C@H](c1c(C(F)(F)F)ccc(N)c1F)C3)C2] | >10,000 | D | 694.2 | ¹H NMR (400 MHz, CDCl₃) δ 7.29 (s, 1H), 6.77 (t, J = 8.4 Hz, 1H), 5.35 (br s, 1H), 5.11-5.05 (m, 1H), 4.99-4.91 (m, 1H), 4.74-4.63 (m, 3H), 4.09 (s, 3H), 4.00- 3.73 (m, 3H), 3.57 (s, 3H), 3.33 (s, 3H), 3.12 (s, 4H), 3.09-3.04 (m, 1H), 3.03-2.84 (m, 3H), 2.22 (br d, J = 4.0 Hz, 6H), 1.95 (br s, 2H) |
| 203 | 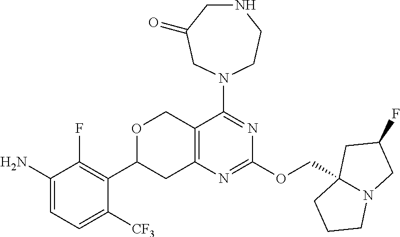 [SMILES: Nc1ccc(C(F)(F)F)c(C2Cc3nc(OC[C@@]45CCCN4C[C@H](F)C5)nc(N4CCNCC(=O)C4)c3CO2)c1F] | >10,000 | D | 583.3 | ¹H NMR (400 MHz, CDCl₃) δ 7.29 (d, J = 10.4 Hz, 1H), 6.78 (t, J = 8.0 Hz, 1H), 5.96- 5.84 (m, 1H), 5.35- 5.18 (m, 1H), 5.17- 5.10 (m, 1H), 4.81- 4.62 (m, 2H), 4.16- 4.06 (m, 3H), 3.95 (d, J = 10.0 Hz, 1H), 3.72-3.48 (m, 5H), 3.43-3.31 (m, 2H), 3.29-3.19 (m, 2H), 3.14 (dd, J = 2.0, 10.4 Hz, 1H), 3.02-2.93 (m, 2H), 2.92-2.84 (m, 1H), 2.76-2.68 (m, 1H), 2.28-2.18 (m, 2H), 2.16-2.07 (m, 2H), 1.94-1.87 (m, 2H) |
| 204 | 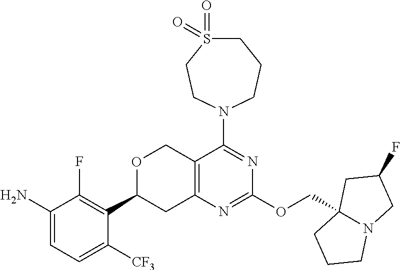 [SMILES: Nc1ccc(C(F)(F)F)c([C@@H]2Cc3nc(OC[C@@]45CCCN4C[C@H](F)C5)nc(N4CCCS(=O)(=O)CC4)c3CO2)c1F] | >10,000 | D | 618.1 | ¹H NMR (400 MHz, methanol-d4) δ 7.25 (d, J = 8.8 Hz, 1H), 6.85 (t, J = 8.4 Hz, 1H), 5.35-5.21 (m, 1H), 5.09 (dd, J = 11.2, 3.6 Hz, 1H), 4.92-4.89 (m, 2H), 4.13-4.02 (m, 3H), 3.98-3.88 (m, 2H), 3.83-3.72 (m, 2H), 3.43-3.38 (m, 1H), 3.36-3.33 (m, 1H), 3.28-3.16 (m, 5H), 3.00 (td, J = 9.2, 5.6 Hz, 1H), 2.82 (dd, J = 17.6, 4.0 Hz, 1H), 2.33-2.07 (m, 5H), 2.02-1.83 (m, 3H). |
| 205 | 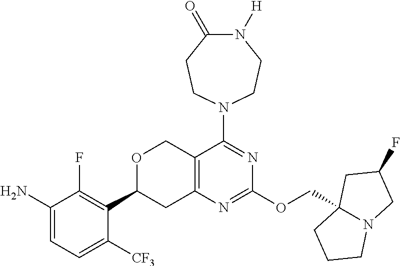 [SMILES: Nc1ccc(C(F)(F)F)c([C@@H]2Cc3nc(OC[C@@]45CCCN4C[C@H](F)C5)nc(N4CCNC(=O)CC4)c3CO2)c1F] | >10,000 | D | 583.3 | ¹H NMR (400 MHz, CDCl₃) δ 7.34-7.29 (m, 1H), 6.86-6.71 (m, 1H), 5.94-5.82 (m, 1H), 5.36-5.19 (m, 1H), 5.10 (br d, J = 2.4 Hz, 1H), 4.80 (br d, J = 2.8 Hz, 2H), 4.02 (s, 4H), 3.73- 3.47 (m, 5H), 3.43- 3.32 (m, 2H), 3.29- 3.20 (m, 2H), 3.20- 3.06 (m, 1H), 3.04- 2.92 (m, 2H), 2.92- 2.83 (m, 1H), 2.78- 2.65 (m, 1H), 2.28 (s, 2H), 2.15-2.11 (m, 1H), 2.06-1.88 (m, 3H) |
| 206 | 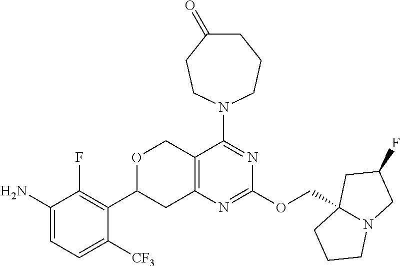 [SMILES: Nc1ccc(C(F)(F)F)c(C2Cc3nc(OC[C@@]45CCCN4C[C@H](F)C5)nc(N4CCCC(=O)CC4)c3CO2)c1F] | >10,000 | D | 582.2 | ¹H NMR (400 MHz, CDCl₃) δ 7.29 (s, 1H), 6.77 (t, J = 8.8 Hz, 1H), 5.34-5.18 (m, 1H), 5.12-5.07 (m, 1H), 4.78 (s, 2H), 4.11- 4.09 (m, 2H), 4.07- 3.92 (m, 3H), 3.69- 3.62 (m, 3H), 3.42- 3.35 (m, 1H), 3.25- 3.20 (m, 2H), 3.18- 3.12 (m, 1H), 2.98- 2.91 (m, 2H), 2.88- 2.82 (m, 1H), 2.75- 2.67 (m, 3H), 2.28- 2.23 (m, 1H), 2.19- 2.08 (m, 2H), 1.99- 1.83 (m, 5H) |
| 207 | 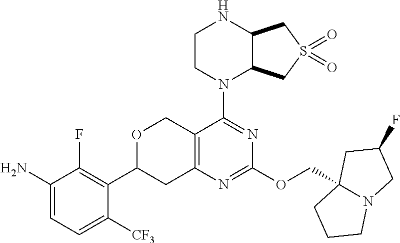 | >10,000 | D | 645.3 | ¹H NMR (400 MHz, CDCl₃) δ 7.30 (s, 1H), 6.78 (t, J = 8.4 Hz, 1H), 5.37-5.18 (m, 1H), 5.14 (dd, J = 10.8, 3.6 Hz, 1H), 5.01- 4.60 (m, 3H), 4.19- 3.96 (m, 4H), 3.93- 3.64 (m, 3H), 3.50- 2.81 (m, 13H), 2.29- 2.04 (m, 3H), 2.01- 1.85 (m, 3H) |
| 208 | 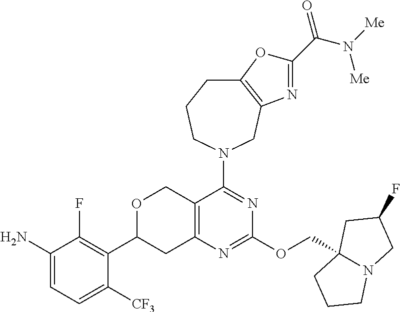 [SMILES: CN(C)C(=O)c1nc2c(o1)CCCN(c1nc(OC[C@@]34CCCN3C[C@H](F)C4)nc3c1COC(c1c(C(F)(F)F)ccc(N)c1F)C3)C2] | 1,441 | D | 678.2 | ¹H NMR (400 MHz, CDCl₃) δ 7.32-7.28 (m, 1H), 6.77 (t, J = 8.4 Hz, 1H), 5.36-5.16 (m, 1H), 5.10 (br dd, J = 3.2, 11.6 Hz, 1H), 4.90-4.81 (m, 1H), 4.80-4.71 (m, 1H), 4.64-4.48 (m, 2H), 4.15-4.03 (m, 3H), 4.00-3.84 (m, 2H), 3.68-3.59 (m, 1H), 3.48 (s, 3H), 3.42- 3.31 (m, 1H), 3.31- 3.19 (m, 2H), 3.18 (br s, 1H), 3.13 (s, 3H), 3.02-2.88 (m, 4H), 2.28-2.08 (m, 4H), 2.04-1.82 (m, 4H) |
| 209 | 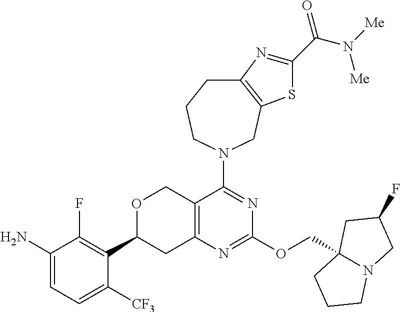 [SMILES: CN(C)C(=O)c1nc2c(s1)CN(c1nc(OC[C@@]34CCCN3C[C@H](F)C4)nc3c1CO[C@H](c1c(C(F)(F)F)ccc(N)c1F)C3)CCC2] | 479 | C | 694.3 | ¹H NMR (400 MHz, CDCl₃) δ 7.29 (s, 1H), 6.77 (t, J = 8.0 Hz, 1H), 5.35-5.21 (m, 1H), 5.10-5.08 (m, 1H), 4.87-4.71 (m, 4H), 4.15-3.99 (m, 4H), 3.84-3.73 (m, 2H), 3.56 (s, 3H), 3.41- 3.08 (m, 8H), 3.06- 2.87 (m, 3H), 2.29- 1.87 (m, 8H) |
| 210 | 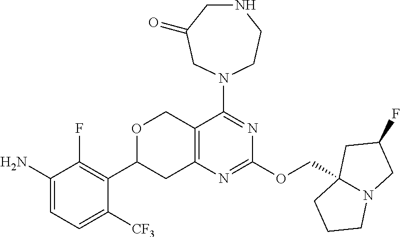 [SMILES: Nc1ccc(C(F)(F)F)c(C2Cc3nc(OC[C@@]45CCCN4C[C@H](F)C5)nc(N4CCNCC(=O)C4)c3CO2)c1F] | >10,000 | D | 583.4 | ¹H NMR (400 MHz, CDCl₃) δ 7.33-7.28 (m, 1H), 6.78 (t, J = 8.4 Hz, 1H), 5.40-5.15 (m, 1H), 5.15-5.07 (m, 1H), 4.75 (br s, 2H), 4.26-4.17 (m, 1H), 4.16-4.04 (m, 3H), 4.01-3.95 (m, 1H), 3.92-3.85 (m, 1H), 3.78 (br dd, J = 2.0, 13.4 Hz, 1H), 3.62-3.53 (m, 1H), 3.52-3.46 (m, 1H), 3.46-3.39 (m, 1H), 3.37 (s, 1H), 3.31- 3.22 (m, 2H), 3.21 (br s, 1H), 3.16 (br d, J = 4.4 Hz, 1H), 3.00-2.84 (m, 3H), 2.23-2.06 (m, 3H), 1.96-1.86 (m, 3H |
| 211a | 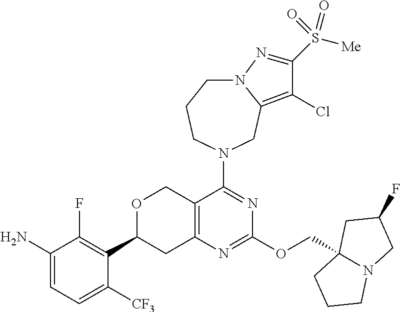 [SMILES: CS(=O)(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2] | 126 | C | 718.2 | ¹H NMR (400 MHz, CDCl₃) δ , 7.30 (s, 1H), 6.78 (t, J = 8.4 Hz, 1H), 5.37-5.10 (m, 2H), 4.92 (br d, J = 13.6 Hz, 1H), 4.82-4.68 (m, 2H), 4.64-4.51 (m, 2H), 4.45-4.35 (m, 1H), 4.11 (s, 2H), 4.04-3.92 (m, 2H), 3.92-3.85 (m, 1H), 3.70-3.61 (m, 1H), 3.37 (dd, J = 11.2, 18.0 Hz, 1H), 3.23 (s, 3H), 3.16- 3.11 (m, 1H), 3.01- 2.91 (m, 2H), 2.25- 2.08 (m, 5H), 1.96- 1.81 (m, 4H), 1.26 (br t, J = 3.3 Hz, 1H) |
| 211b | 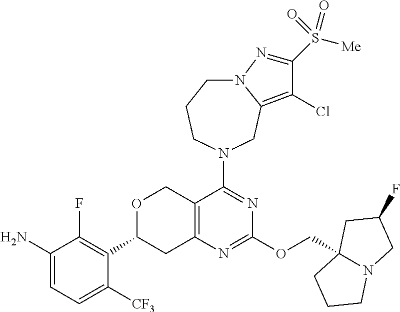 [SMILES: CS(=O)(=O)c1nn2c(c1Cl)CN(c1nc(OC[C@@]34CCCN3C[C@H](F)C4)nc3c1CO[C@@H](c1c(C(F)(F)F)ccc(N)c1F)C3)CCC2] | >10,000 | D | 718.2 | ¹H NMR (400 MHz, CDCl₃) δ , 7.30 (s, 1H), 6.78 (t, J = 8.4 Hz, 1H), 5.34-5.10 (m, 2H), 4.93 (br d, J = 13.6 Hz, 1H), 4.81 (d, J = 16.4 Hz, 2H), 4.63-4.49 (m, 2H), 4.47-4.37 (m, 1H), 4.15-4.10 (m, 2H), 4.07 (s, 1H), 3.94- 3.84 (m, 2H), 3.70- 3.62 (m, 1H), 3.38 (dd, J = 11.2, 18.2 Hz, 1H), 3.22 (s, 3H), 3.14 (s, 1H), 3.01-2.91 (m, 2H), 2.26-2.04 (m, 5H), 1.97-1.83 (m, 4H), 1.27-1.25 (m, 1H) |
| 212a | 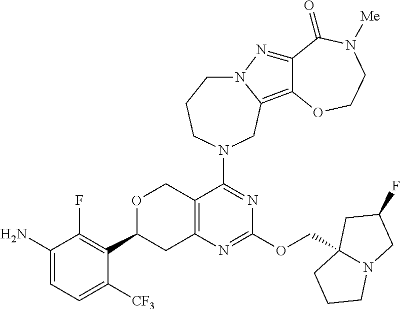 [SMILES: CN1CCOc2c(nn3c2CN(c2nc(OC[C@@]45CCCN4C[C@H](F)C5)nc4c2CO[C@H](c2c(C(F)(F)F)ccc(N)c2F)C4)CCC3)C1=O] | 810.0 | D | 705.3 | ¹H NMR (400 MHz, CDCl₃) δ 7.30 (s, 1H), 6.78 (t, J = 8.4 Hz, 1H), 5.34-5.17 (m, 1H), 5.15-5.02 (m, 2H), 4.72-4.61 (m, 2H), 4.61-4.52 (m, 1H), 4.47-4.34 (m, 2H), 4.34-4.28 (m, 2H), 4.14 (br s, 3H), 4.04 (d, J = 10.4 Hz, 1H), 3.94-3.90 (m, 1H), 3.69-3.53 (m, 3H), 3.39 (br dd, J = 11.6, 17.6 Hz, 1H), 3.28-3.20 (m, 2H), 3.17 (s, 3H), 3.15-3.07 (m, 1H), 3.00-2.88 (m, 2H), 2.45-2.33 (m, 1H), 2.28-2.09 (m, 3H), 2.06-1.99 (m, 1H), 1.95-1.82 (m, 3H) |
| 212b | 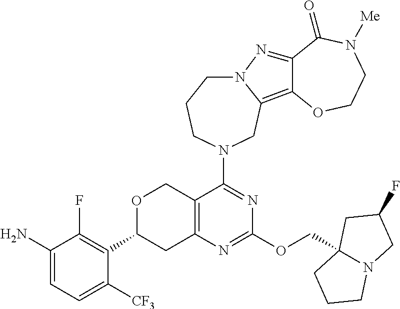 [SMILES: CN1CCOc2c(nn3c2CN(c2nc(OC[C@@]45CCCN4C[C@H](F)C5)nc4c2CO[C@@H](c2c(C(F)(F)F)ccc(N)c2F)C4)CCC3)C1=O] | >10,000 | D | 705.3 | ¹H NMR (400 MHz, CDCl₃) δ 6.78 (t, J = 8.4 Hz, 1H), 5.34-5.17 (m, 1H), 5.13-5.03 (m, 2H), 4.72-4.62 (m, 2H), 4.61-4.54 (m, 1H), 4.45-4.35 (m, 2H), 4.34-4.27 (m, 2H), 4.17-4.08 (m, 3H), 4.06 (d, J = 10.4 Hz, 1H), 3.93- 3.85 (m, 1H), 3.69- 3.53 (m, 3H), 3.40 (br dd, J = 11.6, 17.6 Hz, 1H), 3.26- 3.19 (m, 2H), 3.17 (s, 3H), 3.15-3.05 (m, 1H), 3.00-2.87 (m, 2H), 2.46-2.33 (m, 1H), 2.32-2.10 (m, 3H), 2.08-2.01 (m, 1H), 2.00-1.84 (m, 3H) |
| 213b | 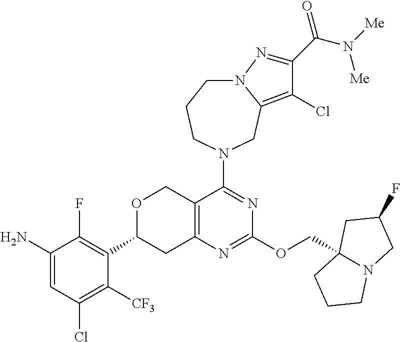 [SMILES: CN(C)C(=O)c1nn2c(c1Cl)CN(c1nc(OC[C@@]34CCCN3C[C@H](F)C4)nc3c1CO[C@@H](c1c(F)c(N)cc(Cl)c1C(F)(F)F)C3)CCC2] | 320 | C | 745.2 | ¹H NMR (400 MHz, CDCl₃) δ 6.84 (d, J = 8.0 Hz, 1H), 5.37-5.21 (m, 1H), 5.19 (br d, J = 3.2 Hz, 1H), 4.96 (br d, J = 13.2 Hz, 1H), 4.82 (d, J = 16.4 Hz, 1H), 4.69 (d, J = 13.6 Hz, 1H), 4.48 (br s, 1H), 4.44 (d, J = 16.8 Hz, 1H), 4.39- 4.30 (m, 1H), 4.17 (s, 2H), 4.11 (d, J = 10.0 Hz, 1H), 3.98- 3.85 (m, 2H), 3.69- 3.60 (m, 1H), 3.37- 3.19 (m, 3H), 3.15 (br s, 1H), 3.10 (s, 6H), 2.97 (br dd, J = 3.2, 17.2 Hz, 2H), 2.25 (br s, 2H), 2.21-2.09 (m, 3H), 1.97-1.80 (m, 3H) |
| 214b | 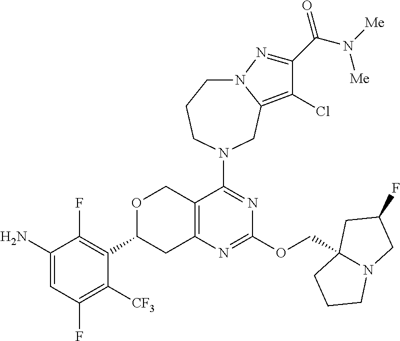 [SMILES: CN(C)C(=O)c1nn2c(c1Cl)CN(c1nc(OC[C@@]34CCCN3C[C@H](F)C4)nc3c1CO[C@@H](c1c(F)c(N)cc(F)c1C(F)(F)F)C3)CCC2] | 1,551 | D | 729.3 | ¹H NMR (400 MHz, CDCl₃) δ 6.51 (dd, J = 7.2, 12.4 Hz, 1H), 5.36- 5.18 (m, 1H), 5.17 (br s, 1H), 4.98 (br d, J = 13.4 Hz, 1H), 4.83 (d, J = 16.8 Hz, 1H), 4.70 (d, J = 13.2 Hz, 1H), 4.56-4.41 (m, 2H), 4.39-4.29 (m, 1H), 4.29-4.20 (m, 2H), 4.10 (d, J = 10.4 Hz, 1H), 3.99-3.88 (m, 2H), 3.70-3.58 (m, 1H), 3.41-3.19 (m, 3H), 3.17-3.05 (m, 7H), 3.01-2.91 (m, 2H), 2.35-2.08 (m, 5H), 1.97-1.79 (m, 3H) |
| 215b | 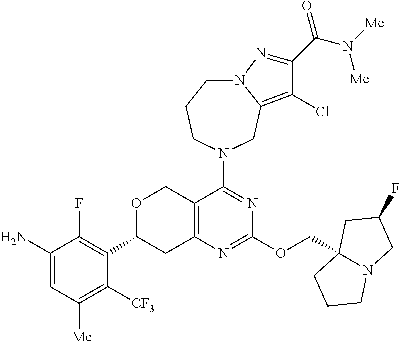 [SMILES: Cc1cc(N)c(F)c([C@H]2Cc3nc(OC[C@@]45CCCN4C[C@H](F)C5)nc(N4CCCn5nc(C(=O)N(C)C)c(Cl)c5C4)c3CO2)c1C(F)(F)F] | 132 | C | 725.3 | ¹H NMR (400 MHz, CDCl₃) δ 6.8 (d, J = 8.4 Hz, 1H), 5.34-5.15 (m, 2H), 4.98 (br d, J = 13.2 Hz, 1H), 4.83 (d, J = 16.8 Hz, 1H), 4.69 (d, J = 13.6 Hz, 1H), 4.53-4.31 (m, 3H), 4.11 (d, J = 10.4 Hz, 1H), 4.00 (s, 2H), 3.97- 3.89 (m, 2H), 3.64 (ddd, J = 3.2, 10.4, 14.0 Hz, 1H), 3.37 (dd, J = 12.0, 18.0 Hz, 1H), 3.29-3.20 (m, 2H), 3.15 (br s, 1H), 3.10 (s, 6H), 2.99-2.92 (m, 2H), 2.39 (q, J = 4.0 Hz, 3H), 2.35-2.26 (m, 1H), 2.26-2.22 (m, 1H), 2.20-2.08 (m, 3H), 1.96-1.80 (m, 3H) |
| 216a | 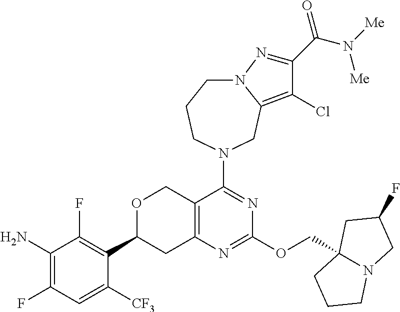 [SMILES: CN(C)C(=O)c1nn2c(c1Cl)CN(c1nc(OC[C@@]34CCCN3C[C@H](F)C4)nc3c1CO[C@H](c1c(C(F)(F)F)cc(F)c(N)c1F)C3)CCC2] | 49 | B | 729.3 | ¹H NMR (400 MHz, CDCl₃) δ ppm 1.83-1.94 (m, 3H) 2.08-2.18 (m, 3H) 2.20-2.25 (m, 1H) 2.28-2.37 (m, 1H) 2.89-3.00 (m, 2H) 3.10 (s, 6H) 3.12-3.19 (m, 1H) 3.19-3.28 (m, 2H) 3.33 (br dd, J = 18.0, 11.6 Hz, 1H) 3.58-3.70 (m, 1H) 3.94 (br d, J = 14.4 Hz, 1H) 3.98-4.08 (m, 2H) 4.13 (s, 2H) 4.29-4.40 (m, 1H) 4.41-4.56 (m, 2H) 4.70 (d, J = 13.6 Hz, 1H) 4.82 (br d, J = 16.4 Hz, 1H) 4.99 (br d, J = 13.6 Hz, 1H) 5.10 (br dd, J = 11.2, 3.2 Hz, 1H) 5.17-5.39 (m, 1H) 7.19 (br d, J = 10.4 Hz, 1H) |
| 216b | 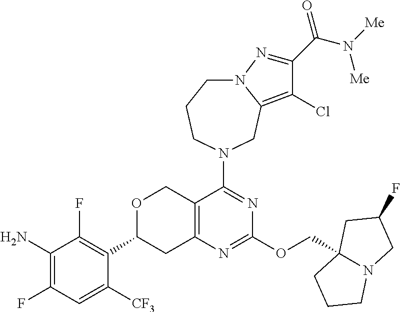 [SMILES: CN(C)C(=O)c1nn2c(c1Cl)CN(c1nc(OC[C@@]34CCCN3C[C@H](F)C4)nc3c1CO[C@@H](c1c(C(F)(F)F)cc(F)c(N)c1F)C3)CCC2] | >10,000 | D | 729.3 | ¹H NMR (400 MHz, CDCl₃) δ ppm 1.83-1.97 (m, 3H) 2.07-2.19 (m, 3H) 2.21-2.26 (m, 1H) 2.28-2.38 (m, 1H) 2.88-3.00 (m, 2H) 3.10 (s, 6H) 3.12-3.19 (m, 1H) 3.19-3.28 (m, 2H) 3.35 (br dd, J = 18.0, 11.6 Hz, 1H) 3.56-3.70 (m, 1H) 3.94 (br d, J = 10.4 Hz, 2H) 4.04-4.20 (m, 3H) 4.29-4.56 (m, 3H) 4.70 (d, J = 13.6 Hz, 1H) 4.85 (d, J = 16.8 Hz, 1H) 5.00 (br d, J = 13.6 Hz, 1H) 5.06-5.15 (m, 1H) 5.16-5.38 (m, 1H) 7.19 (d, J = 10.4 Hz, 1H) |
| 217 | 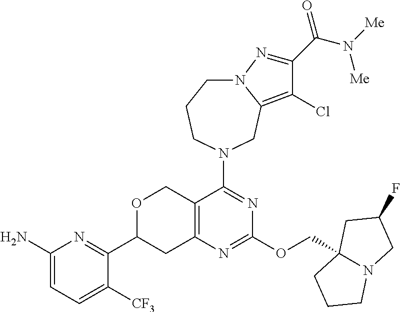 [SMILES: CN(C)C(=O)c1nn2c(c1Cl)CN(c1nc(OC[C@@]34CCCN3C[C@H](F)C4)nc3c1COC(c1nc(N)ccc1C(F)(F)F)C3)CCC2] | 312 | C | 694.3 | ¹H NMR (400 MHz, methanol-d₄) δ 7.68 (d, J = 8.8 Hz, 1H), 6.56 (d, J = 8.8 Hz, 1H), 5.36- 5.22 (m, 1H), 5.11- 5.06 (m, 2H), 4.80- 4.72 (m, 3H), 4.50- 4.45 (m, 1H), 4.38- 4.29 (m, 1H), 4.13 (t, J = 10.0 Hz, 1H), 3.99-3.96 (m, 2H), 3.79-3.74 (m, 1H), 3.40-3.35 (m, 1H), 3.27-3.15 (m, 3H), 3.11-3.08 (m, 6H), 3.02-2.96 (m, 1H), 2.75 (dd, J = 18.0, 3.6 Hz, 1H), 2.38- 2.05 (m, 5H), 1.98- 1.81 (m, 3H). |
| 218 | 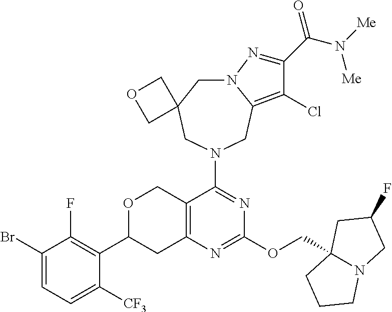 [SMILES: CN(C)C(=O)c1nn2c(c1Cl)CN(c1nc(OC[C@@]34CCCN3C[C@H](F)C4)nc3c1COC(c1c(C(F)(F)F)ccc(Br)c1F)C3)CC1(COC1)C2] | >10,000 | D | 816.2 | ¹H NMR (400 MHz, MeOH-d₄) δ 7.88 (t, J = 7.6 Hz, 1H), 7.55 (d, J = 8.6 Hz, 1H), 5.51- 5.24 (m, 2H), 5.09 (d, J = 13.8 Hz, 1H), 4.99-4.92 (m, 2H), 4.78 (dd, J = 22.0, 14.0 Hz, 2H), 4.71 (d, J = 6.5 Hz, 2H), 4.61 (dd, J = 14.1, 8.8 Hz, 1H), 4.39 (d, J = 6.5 Hz, 1H), 4.37-4.28 (m, 3H), 4.23 (dd, J = 11.0, 5.2 Hz, 1H), 4.20-4.12 (m, 1H), 4.11-3.97 (m, 2H), 3.55-3.35 (m, 4H), 3.29-3.15 (m, 3H), 3.14 (s, 3H), 3.11 (s, 3H), 3.10-3.05 (m, 1H), 2.94 (dd, J = 17.5, 4.6 Hz, 1H), 2.88-2.59 (m, 2H), 2.50-2.13 (m, 3H), 2.12-1.83 (m, 3H). |
| 219 | 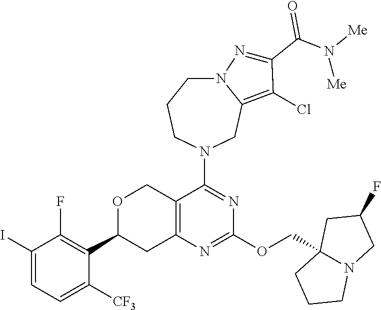 [SMILES: CN(C)C(=O)c1nn2c(c1Cl)CN(c1nc(OC[C@@]34CCCN3C[C@H](F)C4)nc3c1CO[C@H](c1c(C(F)(F)F)ccc(I)c1F)C3)CCC2] | 896 | D | 882.1 | ¹H NMR (400 MHz, MeOH-d₄) δ 8.05 (dd, J = 8.3, 5.9 Hz, 1H), 7.37 (d, J = 8.4 Hz, 1H), 5.46-5.26 (m, 1H), 5.22 (dd, J = 11.1, 4.4 Hz, 1H), 5.04 (d, J = 13.7 Hz, 1H), 4.75 (dd, J = 20.2, 15.0 Hz, 2H), 4.47 (dd, J = 14.2, 7.1 Hz, 1H), 4.29 (dd, J = 14.6, 9.4 Hz, 1H), 4.21 (d, J = 10.9 Hz, 1H), 4.03 (d, J = 11.0 Hz, 1H), 4.00-3.88 (m, 1H), 3.84-3.66 (m, 1H), 3.51-3.33 (m, 3H), 3.20 (dd, J = 17.8, 11.2 Hz, 1H), 3.15-3.12 (m, 1H), 3.11 (s, 3H), 3.09 (s, 3H), 3.08- 3.04 (m, 1H), 2.88 (dd, J = 18.1, 4.5 Hz, 1H), 2.45-2.25 (m, 3H), 2.24-2.10 (m, 3H), 2.09-1.85 (m, 3H). |
| 220 | 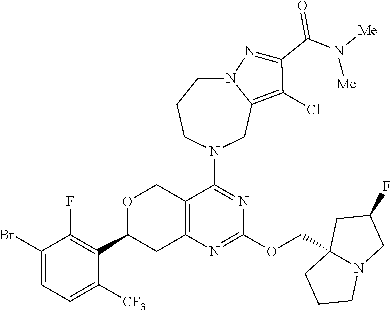 [SMILES: CN(C)C(=O)c1nn2c(c1Cl)CN(c1nc(OC[C@@]34CCCN3C[C@H](F)C4)nc3c1CO[C@H](c1c(C(F)(F)F)ccc(Br)c1F)C3)CCC2] | 1,036 | D | 774.2 | ¹H NMR (400 MHz, MeOH-d₄) δ 7.87 (dd, J = 8.5, 6.5 Hz, 1H), 7.53 (d, J = 8.6 Hz, 1H), 5.43-5.33 (m, 1H), 5.31-5.18 (m, 1H), 5.05 (d, J = 13.7 Hz, 1H), 4.85-4.69 (m, 3H), 4.47 (dd, J = 14.7, 5.9 Hz, 1H), 4.30 (dd, J = 13.2, 10.2 Hz, 1H), 4.15 (d, J = 10.7 Hz, 1H), 4.04-3.92 (m, 2H), 3.82-3.69 (m, 1H), 3.40-3.16 (m, 4H), 3.11 (s, 3H), 3.09 (s, 3H), 3.07- 2.98 (m, 1H), 2.89 (dd, J = 17.9, 4.4 Hz, 1H), 2.44-2.05 (m, 5H), 2.04-1.80 (m, 3H). |
| 221 | 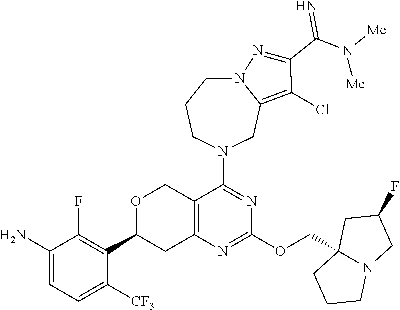 [SMILES: CN(C)C(=N)c1nn2c(c1Cl)CN(c1nc(OC[C@@]34CCCN3C[C@H](F)C4)nc3c1CO[C@H](c1c(C(F)(F)F)ccc(N)c1F)C3)CCC2] | >10,000 | D | 710.3 | ¹H NMR (400 MHz, CDCl₃) δ 8.51 (s, 1H), 7.28 (d, J = 8.9 Hz, 1H), 6.78 (t, J = 8.4 Hz, 1H), 5.40-5.20 (m, 1H), 5.12 (dd, J = 12.0, 3.9 Hz, 1H), 4.87 (d, J = 13.6 Hz, 1H), 4.79-4.62 (m, 2H), 4.60-4.43 (m, 2H), 4.41-4.28 (m, 1H), 4.23-3.96 (m, 3H), 3.89-3.76 (m, 2H), 3.75-3.61 (m, 2H), 3.47-3.28 (m, 6H), 3.24 (s, 3H), 3.10-2.91 (m, 3H), 2.37-2.05 (m, 5H), 2.04-1.88 (m, 2H), 1.87-1.70 (m, 1H). |
| 222 | 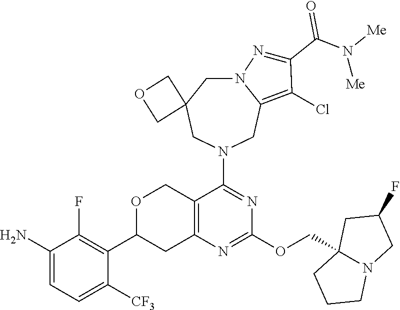 [SMILES: CN(C)C(=O)c1nn2c(c1Cl)CN(c1nc(OC[C@@]34CCCN3C[C@H](F)C4)nc3c1COC(c1c(C(F)(F)F)ccc(N)c1F)C3)CC1(COC1)C2] | >10,000 | D | 753.3 | ¹H NMR (400 MHz, MeOH-d₄) δ 7.26 (d, J = 8.6 Hz, 1H), 6.85 (t, J = 8.5 Hz, 1H), 5.38-5.20 (m, 1H), 5.17 (dd, J = 11.5, 4.6 Hz, 1H), 5.06 (d, J = 13.7 Hz, 1H), 4.95-4.83 (m, 2H), 4.76 (d, J = 5.1 Hz, 1H), 4.74-4.69 (m, 2H), 4.63 (dd, J = 14.2, 10.7 Hz, 1H), 4.41- 4.27 (m, 4H), 4.19- 4.02 (m, 3H), 3.94 (dd, J = 10.6, 9.5 Hz, 1H), 3.30-3.15 (m, 3H), 3.13 (d, J = 2.7 Hz, 3H), 3.10 (s, 3H), 3.06-2.83 (m, 3H), 2.35-2.01 (m, 3H), 2.00-1.77 (m, 3H). |
| 223 | 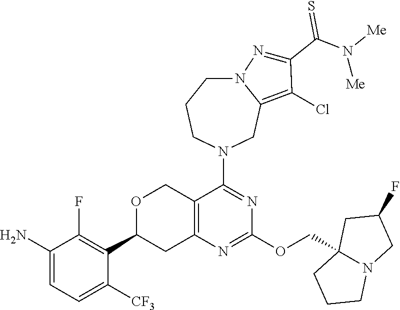 [SMILES: CN(C)C(=S)c1nn2c(c1Cl)CN(c1nc(OC[C@@]34CCCN3C[C@H](F)C4)nc3c1CO[C@H](c1c(C(F)(F)F)ccc(N)c1F)C3)CCC2] | 147 | C | 727.2 | ¹H NMR (400 MHz, MeOH-d₄) δ 7.26 (d, J = 8.6 Hz, 1H), 6.85 (t, J = 8.5 Hz, 1H), 5.57-5.33 (m, 1H), 5.17 (dd, J = 11.3, 4.4 Hz, 1H), 5.05-4.90 (m, 3H), 4.77 (d, J = 13.7 Hz, 1H), 4.61 (d, J = 16.3 Hz, 1H), 4.46-4.31 (m, 2H), 4.24-4.09 (m, 2H), 4.00-3.85 (m, 1H), 3.86-3.73 (m, 2H), 3.72-3.57 (m, 3H), 3.54 (s, 3H), 3.29- 3.26 (m, 1H), 3.23 (s, 3H), 2.87 (dd, J = 17.8, 4.4 Hz, 1H), 2.66-2.30 (m, 4H), 2.28-1.98 (m, 4H). |
| 224 | 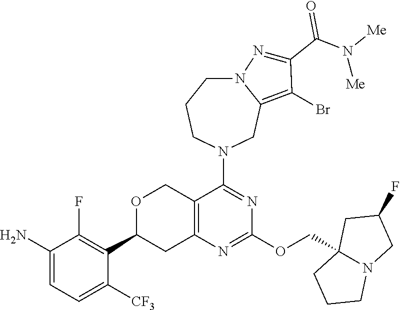 [SMILES: CN(C)C(=O)c1nn2c(c1Br)CN(c1nc(OC[C@@]34CCCN3C[C@H](F)C4)nc3c1CO[C@H](c1c(C(F)(F)F)ccc(N)c1F)C3)CCC2] | 22 | A | 755.2 | ¹H NMR (400 MHz, MeOH-d₄) δ 7.25 (d, J = 8.6 Hz, 1H), 6.85 (t, J = 8.5 Hz, 1H), 5.38-5.19 (m, 1H), 5.15 (dd, J = 11.5, 4.3 Hz, 1H), 5.06 (d, J = 13.7 Hz, 1H), 4.85-4.66 (m, 5H), 4.49 (dd, J = 14.2, 6.8 Hz, 1H), 4.32 (dd, J = 13.9, 10.4 Hz, 1H), 4.12 (d, J = 10.6 Hz, 1H), 4.03-3.98 (m, 1H), 3.95 (d, J = 10.6 Hz, 1H), 3.72 (ddd, J = 14.1, 10.2, 3.8 Hz, 1H), 3.29- 3.12 (m, 5H), 3.09 (s, 3H), 3.08 (s, 3H), 3.04-2.93 (m, 1H), 2.90-2.60 (m, 3H), 2.46-2.01 (m, 5H), 2.00-1.80 (m, 3H). |
| 225 | 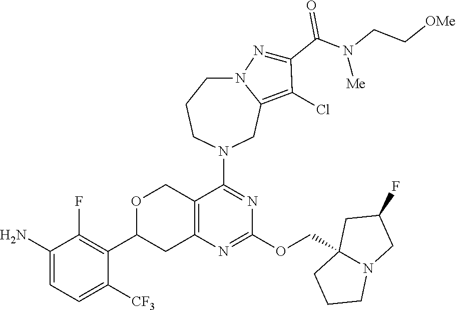 [SMILES: COCCN(C)C(=O)c1nn2c(c1Cl)CN(c1nc(OC[C@@]34CCCN3C[C@H](F)C4)nc3c1COC(c1c(C(F)(F)F)ccc(N)c1F)C3)CCC2] | 82 | B | 755.3 | ¹H NMR (400 MHz, MeOD) δ 7.25 (d, J = 8.7 Hz, 1H), 6.85 (t, J = 8.6 Hz, 1H), 5.43- 5.20 (m, 1H), 5.19- 5.09 (m, 2H), 4.64-4.58 (m, 4H), 4.51-4.43 (m, 1H) 4.40-4.23 (m, 3H), 4.22- 4.08 (m, 1H), 4.04- 3.91 (m, 2H), 3.79-3.60 (m, 3H), 3.54-3.45 (m, 1H), 3.39- 3.32 (m, 3H), 3.22 (s, 1H), 3.17-3.07 (m, 5H), 3.08- 2.99 (m, 1H), 2.93- 2.60 (m, 4H), 2.40-2.29 (m, 1H), 2.27-2.06 (m, 1H), 2.05- 1.92 (m, 2H), 1.35- 1.24 (m, 2H). |
| 226 | 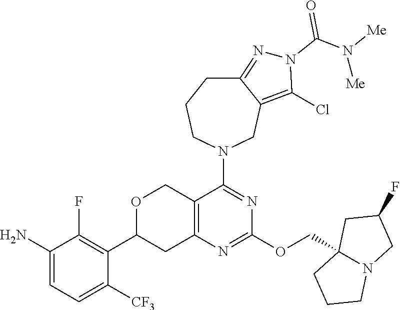 [SMILES: CN(C)C(=O)n1nc2c(c1Cl)CN(c1nc(OC[C@@]34CCCN3C[C@H](F)C4)nc3c1COC(c1c(C(F)(F)F)ccc(N)c1F)C3)CCC2] | 943 | D | 711.3 | ¹H NMR (400 MHz, MeOD) δ 7.23 (d, J = 8.6 Hz, 1H), 6.82 (t, J = 8.5 Hz, 1H), 5.40- 5.17 (m, 1H), 5.10- 5.03 (m, 1H), 4.80-4.65 (m, 3H), 4.60-4.49 (m, 2H), 4.16- 3.98 (m, 3H), 3.97- 3.90 (m, 1H), 3.89-3.80 (m, 1H), 3.63-3.55 (m, 2H), 3.49- 3.44 (m, 1H), 3.25- 3.19 (m, 1H), 3.17 (s, 6H), 3.13- 3.05 (m, 2H), 3.01- 2.84 (m, 3H), 2.82-2.70 (m, 3H), 2.31-2.02 (m, 2H), 1.98- 1.80 (m, 2H), 1.27 (s, 1H) |
| 227 | 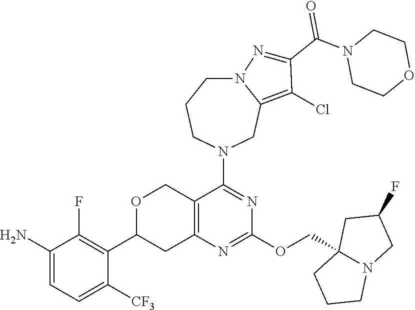 [SMILES: Nc1ccc(C(F)(F)F)c(C2Cc3nc(OC[C@@]45CCCN4C[C@H](F)C5)nc(N4CCCn5nc(C(=O)N6CCOCC6)c(Cl)c5C4)c3CO2)c1F] | 97 | B | 753.3 | ¹H NMR (400 MHz, MeOD) δ 7.26 (d, J = 8.6 Hz, 1H), 6.85 (t, J = 8.5 Hz, 1H), 5.57- 5.34 (m, 1H), 5.24- 5.10 (m, 1H), 5.06-4.95 (m, 1H), 4.81-4.63 (m, 2H), 4.60 (s, 1H), 4.53-4.40 (m, 1H), 4.36- 4.21 (m, 2H), 4.12 (t, J = 10.9 Hz, 1H), 4.03-3.91 (m, 1H), 3.80-3.71 (m, 4H), 3.69- 3.62 (m, 4H), 3.62- 3.46 (m, 3H), 3.27-3.19 (m, 2H), 3.15-3.11 (m, 1H), 2.91- 2.59 (m, 4H), 2.61- 2.44 (m, 1H), 2.42-2.22 (m, 2H), 2.21-2.09 (m, 3H), 2.07-1.98 (m, 1H |
| 228 | 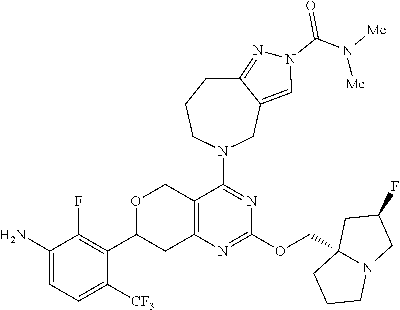 [SMILES: CN(C)C(=O)n1cc2c(n1)CCCN(c1nc(OC[C@@]34CCCN3C[C@H](F)C4)nc3c1COC(c1c(C(F)(F)F)ccc(N)c1F)C3)C2] | 3,074 | D | 677.3 | 1H NMR (400 MHz, MeOD) δ 8.09 (d, J = 3.9 Hz, 1H), 7.25 (d, J = 8.6 Hz, 1H), 6.84 (t, J = 8.5 Hz, 1H), 5.40- 5.18 (m, 1H), 5.14- 5.03 (m, 1H), 4.82-4.67 (m, 3H), 4.61-4.52 (m, 2H), 4.14- 4.01 (m, 3H), 3.98- 3.92 (m, 1H), 3.88 (s, 1H), 3.66- 3.56 (m, 1H), 3.48 (m, 1H), 3.25- 3.20 (m, 1H), 3.17 (s, 6H), 3.14-3.06 (m, 2H), 3.04- 2.88 (m, 3H), 2.83- 2.73 (m, 3H), 2.23-2.05 (m, 2H), 2.01-1.82 (m, 2H), 1.29 (s, 1H) |
| 229 | 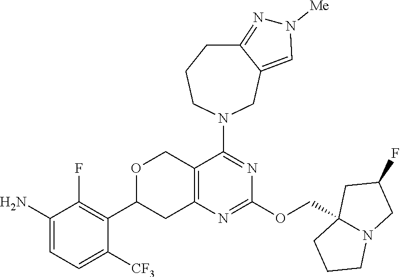 [SMILES: Cn1cc2c(n1)CCCN(c1nc(OC[C@@]34CCCN3C[C@H](F)C4)nc3c1COC(c1c(C(F)(F)F)ccc(N)c1F)C3)C2] | >10,000 | D | 620.3 | 1H NMR (400 MHz, MeOD) δ 7.29 (d, J = 2.9 Hz, 1H), 7.16 (d, J = 8.6 Hz, 1H), 6.82- 6.70 (m, 1H), 5.40 (s, 2H), 5.06-4.96 (m, 1H), 4.66- 4.53 (m, 2H), 4.41- 4.33 (m, 1H), 4.21-4.10 (m, 1H), 3.82-3.72 (m, 1H), 3.67 (s, 2H), 3.64 (d, J = 2.8 Hz, 3H), 3.52- 3.45 (m, 4H), 2.92- 2.81 (m, 1H), 2.77-2.61 (m, 4H), 2.43-2.21 (m, 2H), 2.18- 2.04 (m, 3H), 1.99- 1.87 (m, 4H) |
| 230 | 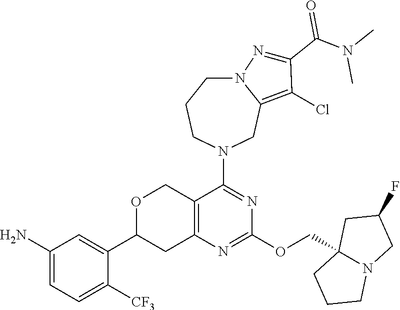 [SMILES: CN(C)C(=O)c1nn2c(c1Cl)CN(c1nc(OC[C@@]34CCCN3C[C@H](F)C4)nc3c1COC(c1cc(N)ccc1C(F)(F)F)C3)CCC2] | 37 | B | 693.3 | ¹H NMR (400 MHz, DMSO-d₆) δ: 7.33 (d, J = 8.8 Hz, 1H), 6.83 (s, 1H), 6.50 (s, 1H), 5.88 (s, 2H), 5.36-5.13 (m, 1H), 5.08-4.98 (m, 1H), 4.91 (br dd, J = 2.8, 10.8 Hz, 1H), 4.75-4.64 (m, 2H), 4.45-4.33 (m, 2H), 3.94-3.78 (m, 3H), 3.75-3.66 (m, 1H), 3.11-3.02 (m, 3H), 2.99 (s, 3H), 2.96 (s, 3H), 2.88-2.65 (m, 4H), 2.26-2.17 (m, 1H), 2.08-1.91 (m, 4H), 1.82-1.66 (m, 3H) |
| 230a | 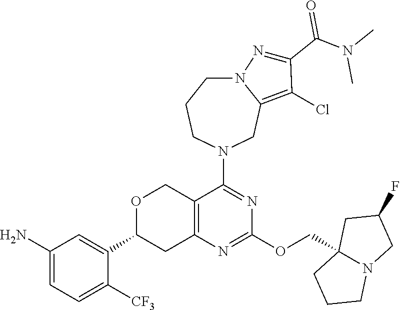 [SMILES: CN(C)C(=O)c1nn2c(c1Cl)CN(c1nc(OC[C@@]34CCCN3C[C@H](F)C4)nc3c1CO[C@@H](c1cc(N)ccc1C(F)(F)F)C3)CCC2] | >10,000 | D | 693.3 | ¹H NMR (400 MHz, DMSO-d₆) δ: 7.33 (d, J = 8.8 Hz, 1H), 6.87 (d, J = 2.0 Hz, 1H), 6.58 (dd, J = 2.0, 8.8 Hz, 1H), 5.89 (s, 2H), 5.32 (br s, 1H), 5.18 (br s, 1H), 5.03 (br d, J = 13.6 Hz, 1H), 4.94-4.79 (m, 2H), 4.75-4.60 (m, 2H), 4.47-4.34 (m, 2H), 3.95 (d, J = 10.4 Hz, 1H), 3.91-3.78 (m, 2H), 3.76-3.63 (m, 1H), 3.06 (br d, J = 7.6 Hz, 2H), 2.98 (s, 3H), 2.96 (s, 3H), 2.77 (br s, 2H), 2.74-2.67 (m, 1H), 2.25-2.15 (m, 1H), 2.09-2.01 (m, 2H), 1.98-1.85 (m, 2H), 1.83-1.68 (m, 3H) |
| 230b | 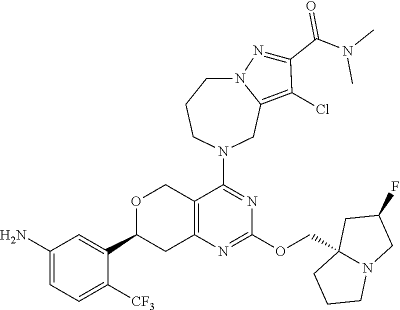 [SMILES: CN(C)C(=O)c1nn2c(c1Cl)CN(c1nc(OC[C@@]34CCCN3C[C@H](F)C4)nc3c1CO[C@H](c1cc(N)ccc1C(F)(F)F)C3)CCC2] | 13 | B | 693.3 | ¹H NMR (400 MHz, DMSO-d₆) δ: 7.33 (d, J = 8.8 Hz, 1H), 6.89-6.84 (m, 1H), 6.57 (dd, J = 2.0, 8.8 Hz, 1H), 5.89 (s, 2H), 5.28 (s, 1H), 5.21-5.14 (m, 1H), 5.07-4.99 (m, 1H), 4.94-4.79 (m, 2H), 4.75-4.64 (m, 2H), 4.47-4.32 (m, 2H), 3.93-3.84 (m, 3H), 3.03 (br s, 2H), 2.99 (s, 3H), 2.96 (s, 3H), 2.83- 2.76 (m, 2H), 2.75- 2.65 (m, 2H), 2.24- 2.16 (m, 1H), 2.10- 2.01 (m, 2H), 1.98- 1.89 (m, 2H), 1.84- 1.71 (m, 3H) |
| 231 | 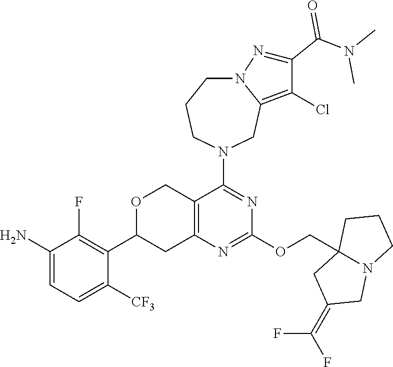 [SMILES: CN(C)C(=O)c1nn2c(c1Cl)CN(c1nc(OCC34CCCN3CC(=C(F)F)C4)nc3c1COC(c1c(C(F)(F)F)ccc(N)c1F)C3)CCC2] | 56 (as mix of 4 diasteromers) | B | 741.18 | ¹H NMR (DMSO- d₆, 400 MHz): δ 7.24 (1H, d, J = 8.7 Hz), 6.81 (1H, t, J = 8.5 Hz), 5.94 (2H, s), 5.01-4.93 (2H, m), 4.85 (1H, m), 4.65-4.60 (2H, m), 4.42-4.31 (2H, m), 3.99-3.84 (3H, m), 3.68-3.55 (2H, m), 3.25 (1H, s), 3.10 (1H, m), 2.95 (3H, s), 2.93 (3H, s), 2.82-2.70 (1H, m), 2.52 (2H, brs), 2.39-2.27 (1H, m), 2.26-2.12 (1H, m), 2.09-1.95 (1H, m), 1.91-1.64 (4H, m) |
| 231a | 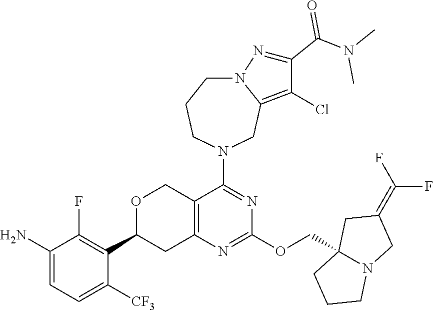 [SMILES: CN(C)C(=O)c1nn2c(c1Cl)CN(c1nc(OC[C@@]34CCCN3CC(=C(F)F)C4)nc3c1CO[C@H](c1c(C(F)(F)F)ccc(N)c1F)C3)CCC2] | 32 | B | 741.2 | ¹H NMR (400 MHz, DMSO) δ 7.25 (d, J = 8.6 Hz, 1H), 6.84 (t, J = 8.5 Hz, 1H), 5.14 (dd, J = 11.2, 4.0 Hz, 1H), 5.04 (d, J = 13.7 Hz, 1H), 4.81 (d, J = 1.9 Hz, 2H), 4.74 (d, J = 13.7 Hz, 1H), 4.52-4.44 (m, 1H), 4.35 (dd, J = 13.6, 9.5 Hz, 1H), 4.11 (dd, J = 24.0, 10.7 Hz, 2H), 4.00 (dt, J = 13.8, 3.9 Hz, 1H), 3.79- 3.71 (m, 2H), 3.39 (d, J = 14.3 Hz, 1H), 3.30-3.24 (m, 1H), 3.14- 3.05 (m, 7H), 2.84 (dd, J = 17.9, 4.2 Hz, 1H), 2.67 (dd, J = 15.5, 4.6 Hz, 2H), 2.44 (d, J = 15.9 Hz, 1H), 2.34 (dd, J = 13.2, 6.4 Hz, 1H), 2.20-2.13 (m, 1H), 2.04 (dd, J = 9.2, 5.9 Hz, 1H), 1.98-1.91 (m, 1H), 1.89-1.82 (m, 2H) |
| 231b | 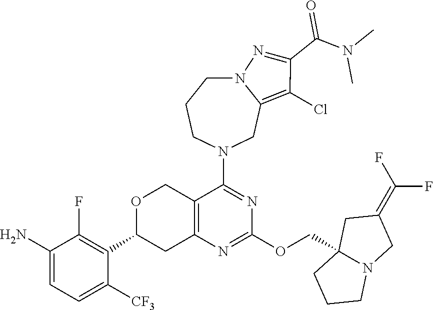 [SMILES: CN(C)C(=O)c1nn2c(c1Cl)CN(c1nc(OC[C@@]34CCCN3CC(=C(F)F)C4)nc3c1CO[C@@H](c1c(C(F)(F)F)ccc(N)c1F)C3)CCC2] | >10,000 | D | 741.2 | ¹H NMR (400 MHz, DMSO) δ 7.25 (d, J = 8.6 Hz, 1H), 6.85 (t, J = 8.5 Hz, 1H), 5.14 (dd, J = 11.2, 4.0 Hz, 1H), 5.04 (d, J = 13.7 Hz, 1H), 4.81 (d, J = 5.0 Hz, 2H), 4.74 (t, J = 8.4 Hz, 1H), 4.52-4.44 (m, 1H), 4.36 (dd, J = 13.5, 9.5 Hz, 1H), 4.21 (d, J = 10.7 Hz, 1H), 3.99 (d, J = 10.8 Hz, 2H), 3.79-3.71 (m, 2H), 3.39 (d, J = 14.2 Hz, 1H), 3.30- 3.24 (m, 1H), 3.08 (t, J = 4.9 Hz, 7H), 2.84 (dd, J = 17.8, 4.2 Hz, 1H), 2.74-2.62 (m, 2H), 2.46 (d, J = 15.8 Hz, 1H), 2.35 (dd, J = 19.6, 9.8 Hz, 1H), 2.16 (dd, J = 11.7, 6.9 Hz, 1H), 2.06 (dd, J = 9.5, 5.8 Hz, 1H), 1.94 (ddd, J = 8.6, 6.6, 3.1 Hz, 1H), 1.90- 1.83 (m, 2H) |
| 232 | 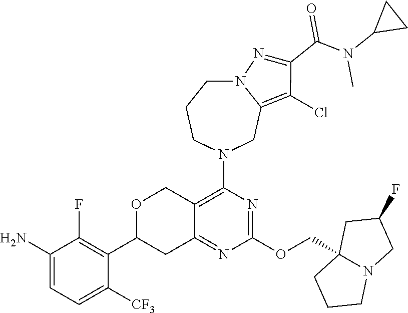 [SMILES: CN(C(=O)c1nn2c(c1Cl)CN(c1nc(OC[C@@]34CCCN3C[C@H](F)C4)nc3c1COC(c1c(C(F)(F)F)ccc(N)c1F)C3)CCC2)C1CC1] | 247 As mix of diastereo- mers; individual diastereo- mers: 166 (S) and >1,000 (R) | C | 737.3 | ¹H NMR (400 MHz, MeOH-d₄) δ 8.43 (br s, 1H), 7.26 (d, J = 8.5 Hz, 1H), 6.86 (t, J = 8.5 Hz, 1H), 5.68-5.41 (m, 1H), 5.16 (dd, J = 11.4, 4.3 Hz, 1H), 5.02 (d, J = 13.9 Hz, 1H), 4.98-4.90 (m, 1H), 4.77 (dd, J = 13.8, 3.2 Hz, 1H), 4.66 (d, J = 16.4 Hz, 1H), 4.53-4.37 (m, 2H), 4.32-4.14 (m, 2H), 4.05-3.66 (m, 5H), 3.44-3.33 (m, 1H), 3.30-3.22 (m, 1H), 3.17-3.01 (m, 3H), 3.00-2.92 (m, 1H), 2.87 (dd, J = 18.0, 4.4 Hz, 1H), 2.71-2.46 (m, 2H), 2.45-2.32 (m, 2H), 2.31-2.20 (m, 2H), 2.19-2.05 (m, 2H), 1.00-0.69 (m, 1H), 0.68-0.40 (m, 3H). |
| 233 | 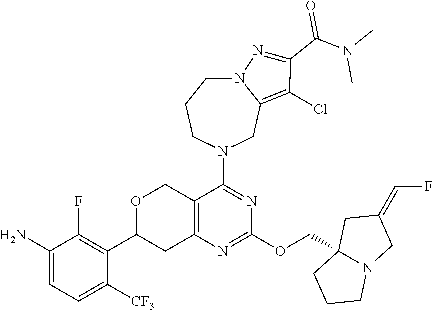 [SMILES: CN(C)C(=O)c1nn2c(c1Cl)CN(c1nc(OC[C@@]34CCCN3C/C(=C\F)C4)nc3c1COC(c1c(C(F)(F)F)ccc(N)c1F)C3)CCC2] | 30 | A | 723.2 | ¹H NMR (400 MHz, CD₃OD) δ 7.29 (d, J = 8.6 Hz, 1H), 7.04-6.79 (m, 2H), 5.19 (dd, J = 11.1, 4.1 Hz, 1H), 5.04 (d, J = 10.3 Hz, 2H), 4.83- 4.68 (m, 2H), 4.65- 4.36 (m, 4H), 4.27 (d, J = 9.9 Hz, 1H), 4.15 (d, J = 14.5 Hz, 1H), 4.02 (s, 1H), 3.93-3.76 (m, 2H), 3.37 (s, 1H), 3.28 (d, J = 6.9 Hz, 1H), 3.18- 3.10 (m, 6H), 3.07- 2.90 (m, 2H), 2.80 (t, J = 15.6 Hz, 1H), 2.49-2.33 (m, 2H), 2.31- 2.12 (m, 4H). |
| 237 | 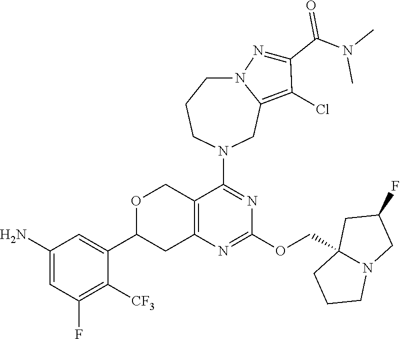 [SMILES: CN(C)C(=O)c1nn2c(c1Cl)CN(c1nc(OC[C@@]34CCCN3C[C@H](F)C4)nc3c1COC(c1cc(N)cc(F)c1C(F)(F)F)C3)CCC2] | 20.4 | A | 711.4 | ¹H NMR (400 MHz, DMSO-d₆) δ: 6.73 (s, 1H), 6.38 (dd, J = 1.6, 14.8 Hz, 1H), 6.27 (s, 2H), 5.73-5.36 (m, 1H), 5.14-4.94 (m, 2H), 4.85-4.66 (m, 3H), 4.45 (br dd, J = 7.2, 13.6 Hz, 1H), 4.39-4.10 (m, 3H), 3.97-3.50 (m, 5H), 3.28-3.13 (m, 1H), 3.04-2.95 (m, 6H), 2.88 (br d, J = 18.0 Hz, 1H), 2.69 (br dd, J = 10.8, 17.6 Hz, 1H), 2.49-2.29 (m, 2H), 2.29-1.91 (m, 6H) |
| 238 | 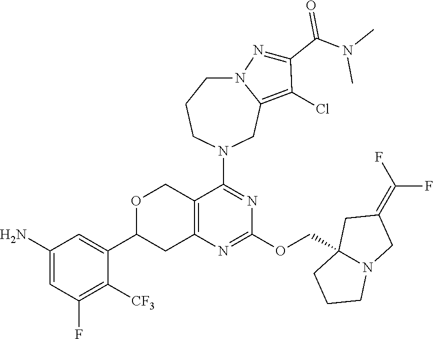 [SMILES: CN(C)C(=O)c1nn2c(c1Cl)CN(c1nc(OC[C@@]34CCCN3CC(=C(F)F)C4)nc3c1COC(c1cc(N)cc(F)c1C(F)(F)F)C3)CCC2] | 29.3 | A | 741.3 | ¹H NMR (400 MHz, DMSO-d₆) δ: 6.69 (s, 1H), 6.42- 6.30 (m, 1H), 6.23 (s, 2H), 5.15-4.91 (m, 2H), 4.81-4.64 (m, 3H), 4.53-4.00 (m, 5H), 3.97-3.59 (m, 3H), 3.57-3.35 (m, 1H), 3.19-3.01 (m, 1H), 2.94 (d, J = 12.8 Hz, 6H), 2.88-2.58 (m, 4H), 2.24-1.81 (m, 6H) |
| 239 | 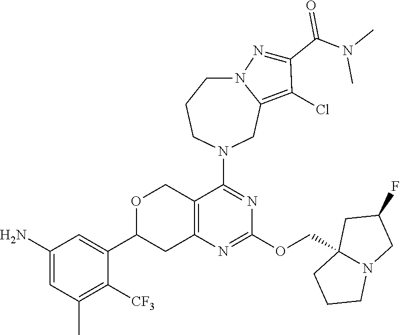 [SMILES: Cc1cc(N)cc(C2Cc3nc(OC[C@@]45CCCN4C[C@H](F)C5)nc(N4CCCn5nc(C(=O)N(C)C)c(Cl)c5C4)c3CO2)c1C(F)(F)F] | 12.8 | A | 707.2 | ¹H NMR (400 MHz, DMSO-d₆) δ: 6.75 (s, 1H), 6.43 (s, 1H), 5.78 (s, 2H), 5.40 (s, 1H), 5.12-4.93 (m, 2H), 4.84-4.67 (m, 3H), 4.44 (dd, J = 7.2, 12.8 Hz, 1H), 4.38- 4.06 (m, 3H), 3.96- 3.69 (m, 3H), 3.60- 3.48 (m, 1H), 3.30 (s, 1H), 3.18-3.08 (m, 1H), 2.99 (d, J = 13.2 Hz, 6H), 2.90-2.81 (m, 1H), 2.72-2.64 (m, 1H), 2.38-2.18 (m, 6H), 2.17-1.81 (m, 5H) |
| 241 | 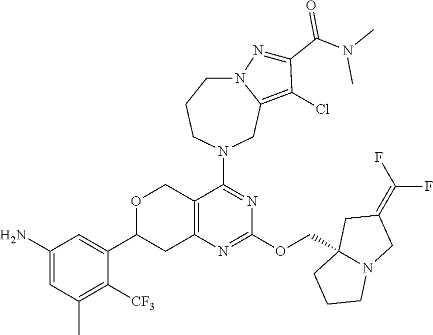 [SMILES: Cc1cc(N)cc(C2Cc3nc(OC[C@@]45CCCN4CC(=C(F)F)C5)nc(N4CCCn5nc(C(=O)N(C)C)c(Cl)c5C4)c3CO2)c1C(F)(F)F] | 58.9 | B | 737.1 | ¹H NMR (400 MHz, DMSO-d₆) δ: 6.75 (s, 1H), 6.43 (s, 1H), 4.95-5.12 (m, 2H), 4.82- 4.71 (m, 3H), 4.60- 4.37 (m, 3H), 4.36-4.15 (m, 3H), 4.13-4.01 (m, 1H), 3.95-3.69 (m, 3H), 3.28 (dd, J = 6.4, 4.4 Hz, 1H), 3.10 (d, J =4.4 Hz, 1H), 2.99 (d, J = 14.4 Hz, 6H), 2.93-2.77 (m, 3H), 2.68 (dd, J = 3.2, 1.6 Hz, 1H), 2.31 (d, J = 3.2 Hz, 3H), 2.26-2.10 (m, 4H), 2.07- 1.98 (m, 2H) |
| 243 | 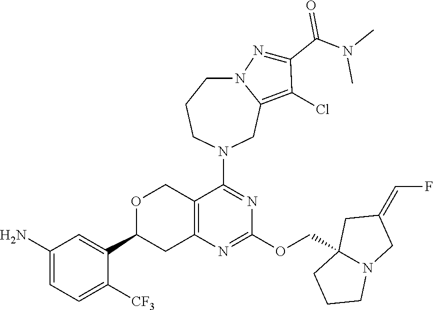 [SMILES: CN(C)C(=O)c1nn2c(c1Cl)CN(c1nc(OC[C@@]34CCCN3C/C(=C\F)C4)nc3c1CO[C@H](c1cc(N)ccc1C(F)(F)F)C3)CCC2] | 10.7 | A | 705.4 | ¹H NMR (400 MHz, DMSO-d₆) δ: 7.33 (d, J = 8.8 Hz, 1H), 6.80-6.61 (m, 2H), 6.58 (dd, J = 2.0, 8.8 Hz, 1H), 5.92-5.84 (m, 2H), 5.13-4.97 (m, 1H), 4.96-4.81 (m, 2H), 4.76-4.65 (m, 2H), 4.48-4.31 (m, 2H), 4.10-3.85 (m, 3H), 3.84-3.67 (m, 2H), 3.65-3.43 (m, 1H), 3.25-3.08 (m, 1H), 2.99 (s, 3H), 2.96 (s, 3H), 2.88-2.80 (m, 1H), 2.78-2.61 (m, 2H), 2.47-2.10 (m, 3H), 2.08-1.90 (m, 3H), 1.86-1.71 (m, 2H) |
| 244 | 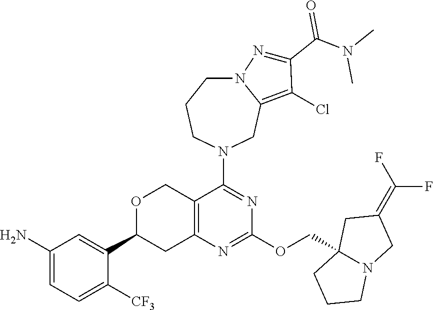 [SMILES: CN(C)C(=O)c1nn2c(c1Cl)CN(c1nc(OC[C@@]34CCCN3CC(=C(F)F)C4)nc3c1CO[C@H](c1cc(N)ccc1C(F)(F)F)C3)CCC2] | 22.2 | A | 723.4 | ¹H NMR (400 MHz, DMSO-d₆) δ: 7.33 (d, J = 8.8 Hz, 1H), 6.87 (d, J = 2.0 Hz, 1H), 6.62 (s, 1H), 5.88 (s, 2H), 5.09-4.96 (m, 1H), 4.95-4.80 (m, 2H), 4.78-4.65 (m, 2H), 4.48-4.29 (m, 2H), 4.14-4.00 (m, 1H), 3.99-3.84 (m, 2H), 3.80-3.64 (m, 2H), 2.99 (s, 3H), 2.96 (s, 3H), 2.86- 2.78 (m, 1H), 2.76- 2.58 (m, 3H), 2.30- 2.13 (m, 2H), 2.10- 1.97 (m, 2H), 1.96- 1.86 (m, 2H), 1.84- 1.71 (m, 3H) |
| 246 | 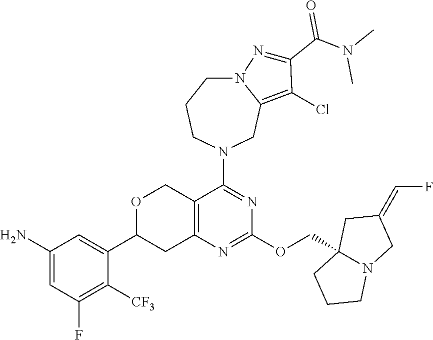 [SMILES: CN(C)C(=O)c1nn2c(c1Cl)CN(c1nc(OC[C@@]34CCCN3C/C(=C\F)C4)nc3c1COC(c1cc(N)cc(F)c1C(F)(F)F)C3)CCC2] | 15.9 | A | 723.4 | ¹H NMR (400 MHz, DMSO-d₆) δ: 7.01-6.75 (m, 1H), 6.72 (d, J = 1.6 Hz, 1H), 6.45-6.32 (m, 1H), 6.26 (s, 2H), 5.09-4.94 (m, 2H), 4.86-4.65 (m, 3H), 4.57-4.00 (m, 4H), 3.96-3.66 (m, 3H), 3.09 (br d, J = 7.6 Hz, 2H), 2.97 (d, J = 10.4 Hz, 6H), 2.91-2.61 (m, 4H), 2.29-2.14 (m, 2H), 2.13-1.69 (m, 5H) |
| 247b | 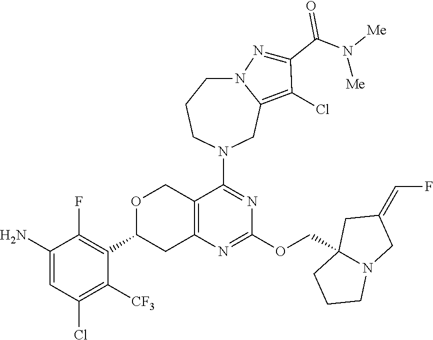 [SMILES: CN(C)C(=O)c1nn2c(c1Cl)CN(c1nc(OC[C@@]34CCCN3C/C(=C\F)C4)nc3c1CO[C@@H](c1c(F)c(N)cc(Cl)c1C(F)(F)F)C3)CCC2] | 615 | D | 757.2 | ¹H NMR (400 MHz, CDCl₃) δ 6.84 (d, J = 8.0 Hz, 1H), 6.63-6.33 (m, 1H), 5.21 (br dd, J = 4.0, 10.8 Hz, 1H), 4.99 (br d, J = 13.2 Hz, 1H), 4.85 (d, J = 16.4 Hz, 1H), 4.68 (d, J = 13.6 Hz, 1H), 4.55-4.48 (m, 1H), 4.41 (d, J = 16.4 Hz, 1H), 4.38-4.32 (m, 1H), 4.18 (s, 2H), 4.09- 3.92 (m, 3H), 3.84 (br d, J = 15.2 Hz, 1H), 3.65 (ddd, J = 3.2, 10.8, 13.8 Hz, 1H), 3.44-3.29 (m, 2H), 3.18-3.12 (m, 1H), 3.10 (s, 6H), 3.03-2.95 (m, 1H), 2.73-2.60 (m, 2H), 2.37-2.27 (m, 2H), 2.18-2.07 (m, 2H), 1.93-1.85 (m, 2H), 1.77 (br d, J = 4.8 Hz, 1H) |
| 248b | 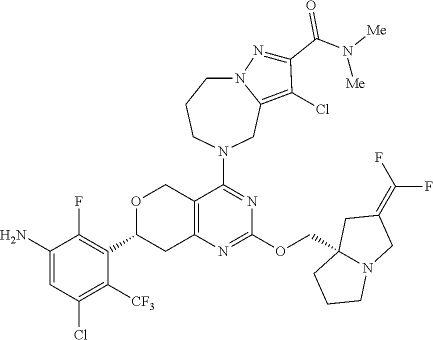 [SMILES: CN(C)C(=O)c1nn2c(c1Cl)CN(c1nc(OC[C@@]34CCCN3CC(=C(F)F)C4)nc3c1CO[C@@H](c1c(F)c(N)cc(Cl)c1C(F)(F)F)C3)CCC2] | >2,500 | D | 775.3 | ¹H NMR (400 MHz, CDCl₃) δ 6.84 (d, J = 7.6 Hz, 1H), 5.26-5.17 (m, 1H), 4.97 (br d, J = 13.2 Hz, 1H), 4.85 (br d, J = 16.4 Hz, 1H), 4.68 (d, J = 13.6 Hz, 1H), 4.55- 4.47 (m, 1H), 4.42 (br d, J = 16.8 Hz, 1H), 4.39-4.32 (m, 1H), 4.18 (s, 2H), 4.10-3.99 (m, 2H), 3.96-3.88 (m, 1H), 3.79-3.61 (m, 2H), 3.40-3.28 (m, 2H), 3.17-3.12 (m, 1H), 3.10 (s, 6H), 3.02- 2.94 (m, 1H), 2.77 (br d, J = 15.6 Hz, 1H), 2.66-2.58 (m, 1H), 2.41-2.29 (m, 2H), 2.20-2.08 (m, 2H), 1.95-1.87 (m, 2H), 1.82 (br d, J = 8.4 Hz, 1H). |
| 249 | 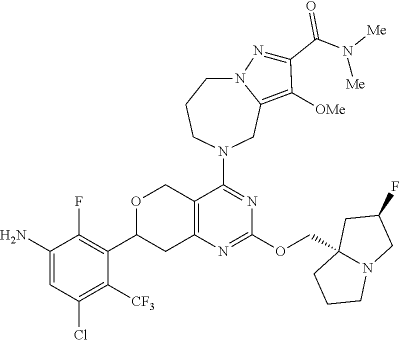 [SMILES: COc1c(C(=O)N(C)C)nn2c1CN(c1nc(OC[C@@]34CCCN3C[C@H](F)C4)nc3c1COC(c1c(F)c(N)cc(Cl)c1C(F)(F)F)C3)CCC2] | 113 | C | 741.3 | ¹H NMR (400 MHz, CDCl₃) δ 6.84 (d, J = 7.6 Hz, 1H), 5.35-5.16 (m, 2H), 4.99 (br d, J = 13.6 Hz, 1H), 4.76- 4.63 (m, 2H), 4.49- 4.40 (m, 1H), 4.38- 4.27 (m, 2H), 4.20 (s, 2H), 4.10-3.99 (m, 2H), 3.98-3.91 (m, 1H), 3.79 (d, J = 1.2 Hz, 3H), 3.71- 3.60 (m, 1H), 3.30 (br dd, J = 4.0, 12.0 Hz, 1H), 3.27-3.20 (m, 2H), 3.14 (br d, J = 13.2 Hz, 1H), 3.09 (s, 6H), 3.01- 2.88 (m, 2H), 2.41- 2.22 (m, 2H), 2.20 (br s, 2H), 2.08- 2.00 (m, 1H), 1.97- 1.81 (m, 3H) |
| 250a | 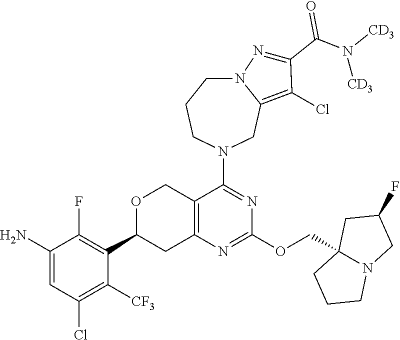 [SMILES: [2H]C([2H])([2H])N(C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2)C([2H])([2H])[2H]] | | | 751.3 | ¹H NMR (400 MHz, CDCl₃) δ 6.84 (d, J = 7.6 Hz, 1H), 5.49-5.27 (m, 1H), 5.20 (br dd, J = 4.0, 11.6 Hz, 1H), 4.94 (br d, J = 13.6 Hz, 1H), 4.70 (d, J = 13.2 Hz, 2H), 4.62-4.53 (m, 1H), 4.46 (br d, J = 6.8 Hz, 1H), 4.50 (br d, J = 6.0 Hz, 1H), 4.32 (br dd, J = 10.8, 12.8 Hz, 2H), 4.20 (s, 2H), 4.16- 4.09 (m, 1H), 3.94- 3.87 (m, 1H), 3.68- 3.60 (m, 1H), 3.32 (br dd, J = 11.2, 17.8 Hz, 2H), 3.26- 3.01 (m, 2H), 2.97 (br dd, J = 4.0, 18.4 Hz, 1H), 2.48-2.22 (m, 4H), 2.20-2.01 (m, 4H) |
| 250b | 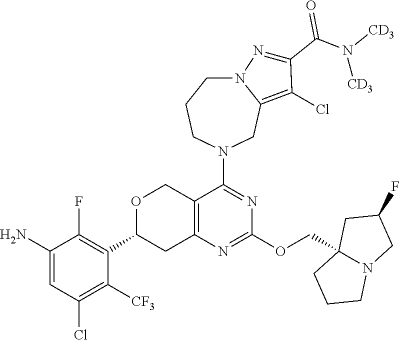 [SMILES: [2H]C([2H])([2H])N(C(=O)c1nn2c(c1Cl)CN(c1nc(OC[C@@]34CCCN3C[C@H](F)C4)nc3c1CO[C@@H](c1c(F)c(N)cc(Cl)c1C(F)(F)F)C3)CCC2)C([2H])([2H])[2H]] | | A | 751.3 | ¹H NMR (400 MHz, CDCl₃) δ 6.84 (d, J = 8.0 Hz, 1H), 5.46-5.28 (m, 1H), 5.21 (br dd, J = 4.2, 11.2 Hz, 1H), 4.94 (br d, J = 13.2 Hz, 1H), 4.77-4.66 (m, 2H), 4.55-4.44 (m, 2H), 4.38-4.30 (m, 1H), 4.25 (br s, 1H), 4.21 (s, 2H), 4.16 (br s, 1H), 3.94-3.89 (m, 1H), 3.72-3.60 (m, 2H), 3.33 (br dd, J = 11.6, 17.6 Hz, 2H), 3.21-3.12 (m, 1H), 3.09 (br d, J = 6.0 Hz, 1H), 2.97 (br dd, J = 3.2, 18.4 Hz, 1H), 2.39-2.22 (m, 4H), 2.04 (br d, J = 5.6 Hz, 4H) |
| 401 | 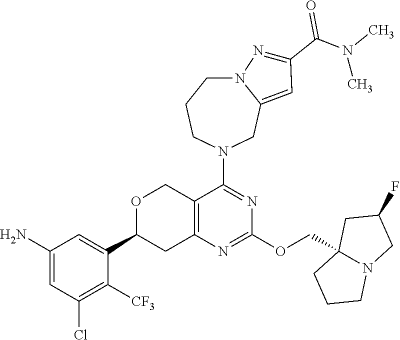 [SMILES: CN(C)C(=O)c1cc2n(n1)CCCN(c1nc(OC[C@@]34CCCN3C[C@H](F)C4)nc3c1CO[C@H](c1cc(N)cc(Cl)c1C(F)(F)F)C3)C2] | | A | 711.4 | ¹H NMR (400 MHz, DMSO-d6) δ: 6.89 (d, J = 1.6 Hz, 1H), 6.69 (d, J = 2.4 Hz, 1H), 6.58 (s, 1H), 6.20 (s, 2H), 5.35-5.15 (m, 1H), 5.03 (br d, J = 12.8 Hz, 2H), 4.75- 4.66 (m, 3H), 4.48- 4.41 (m, 2H), 3.93- 3.89 (m, 1H), 3.82 (br s, 3H), 3.25 (s, 3H), 3.08-3.03 (m, 2H), 2.97 (br d, J = 3.2 Hz, 1H), 2.93 (s, 3H), 2.84-2.78 (m, 2H), 2.69-2.58 (m, 2H), 2.14-2.04 (m, 2H), 2.00-1.92 (m, 3H), 1.80-1.72 (m, 2H) |
| 402 | 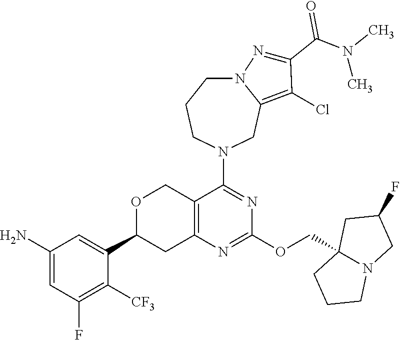 [SMILES: CN(C)C(=O)c1nn2c(c1Cl)CN(c1nc(OC[C@@]34CCCN3C[C@H](F)C4)nc3c1CO[C@H](c1cc(N)cc(F)c1C(F)(F)F)C3)CCC2] | | A | 711.2 | ¹H NMR (400 MHz, DMSO-d6) δ: 6.73 (s, 1H), 6.38 (dd, J = 1.6, 14.8 Hz, 1H), 6.27 (s, 2H), 5.66-5.43 (m, 1H), 5.13-4.94 (m, 2H), 4.83-4.70 (m, 3H), 4.51-4.40 (m, 1H), 4.39-4.11 (m, 3H), 3.95-3.53 (m, 5H), 3.31-3.18 (m, 1H), 2.99 (d, J = 13.2 Hz, 6H), 2.93- 2.82 (m, 1H), 2.69 (br dd, J = 10.8, 17.6 Hz, 1H), 2.49- 2.30 (m, 2H), 2.29- 1.91 (m, 6H) |
| 403 | 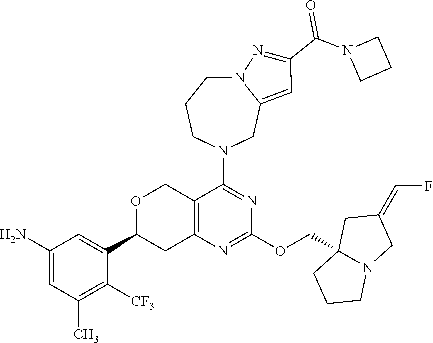 [SMILES: Cc1cc(N)cc([C@@H]2Cc3nc(OC[C@@]45CCCN4C/C(=C\F)C5)nc(N4CCCn5nc(C(=O)N6CCC6)cc5C4)c3CO2)c1C(F)(F)F] | | A | 696.4 | ¹H NMR (400 MHz, DMSO-d6) δ: 6.84-6.61 (m, 3H), 6.42 (s, 1H), 5.75 (s, 2H), 4.97 (m, J = 3.2 Hz, 1H), 4.74-4.61 (m, 3H), 4.48-4.38 (m, 4H), 3.96 (m, J = 7.6 Hz, 2H), 3.89-3.79 (m, 4H), 3.66 (m, J = 14.4 Hz, 1H), 3.05- 2.90 (m, 2H), 2.83- 2.77 (m, 1H), 2.68- 2.56 (m, 2H), 2.32- 2.02 (m, 9H), 1.95- 1.62 (m, 6H) |
| 404 | 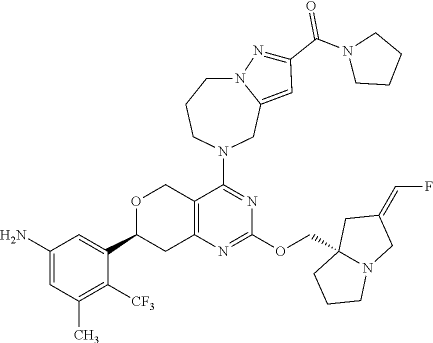 [SMILES: Cc1cc(N)cc([C@@H]2Cc3nc(OC[C@@]45CCCN4C/C(=C\F)C5)nc(N4CCCn5nc(C(=O)N6CCCC6)cc5C4)c3CO2)c1C(F)(F)F] | | A | 711.5 | ¹H NMR (400 MHz, DMSO-d6) δ: 7.06 (d, J = 84 Hz, 1H), 6.75 (s, 1H), 6.71 (s ,1H), 6.40 (s, 1H), 5.65 (s, 2H), 4.95-5.10 (m, 2H), 4.66-4.81 (m, 3H), 4.36-4.52 (m, 3H), 4.17-4.31 (m, 2H), 3.95-4.05 (m, 1H), 3.83-3.91 (m, 2H), 3.77-3.81 (m, 2H), 3.49-3.62 (m, 1H), 3.43 (m t, J = 6.8 Hz, 2H), 3.15-3.21 (m, 1H), 2.75-2.86 (m, 2H), 2.61-2.70 (m, 2H), 2.30 (m, J = 3.2 Hz, 3H), 2.08-2.17 (m, 3H), 1.92-2.03 (m, 3H), 1.78-1.89 (m, 4H) |
| 405 | 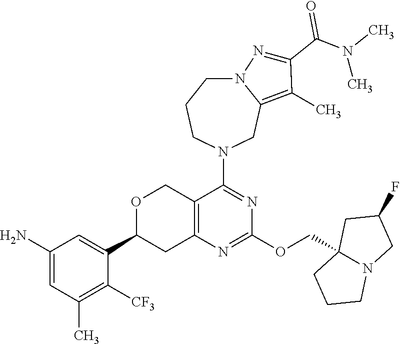 [SMILES: Cc1cc(N)cc([C@@H]2Cc3nc(OC[C@@]45CCCN4C[C@H](F)C5)nc(N4CCCn5nc(C(=O)N(C)C)c(C)c5C4)c3CO2)c1C(F)(F)F] | | A | 687.4 | ¹H NMR (400 MHz, DMSO-d6) δ: 6.75 (s, 1H), 6.41 (s, 1H), 5.65 (s, 2H), 5.16-5.33 (m, 1H), 4.90-5.02 (m, 2H), 4.61-4.77 (m, 3H), 4.31-4.43 (m, 2H), 3.75-3.92 (m, 5H), 3.06 (m, 5H), 2.97 (m, 1H), 2.93 (m, 3H), 2.77-2.84 (m, 2H), 2.30 (m d, J = 3.2 Hz, 3H), 2.11 (m, 3H), 2.02- 2.08 (m, 2H), 1.89- 1.98 (m, 3H), 1.79- 1.85 (m, 1H), 1.68- 1.76 (m, 2H) |
| 406 | 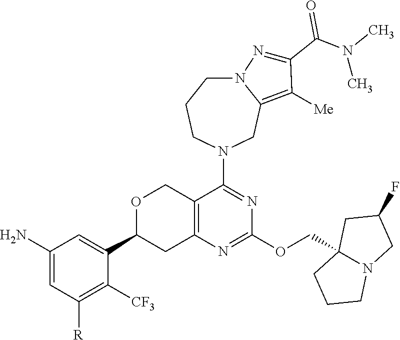 | | A | 691.4 | ¹H NMR (400 MHz, DMSO-d6) δ: 6.72 (s, 1H), 6.38 (dd, J = 1.6, 14.8 Hz, 1H), 6.24 (s, 2H), 5.33-5.14 (m, 1H), 5.04- 4.91 (m, 2H), 4.79- 4.60 (m, 3H), 4.46-4.27 (m, 2H), 3.90-3.75 (m, 4H), 3.11- 3.01 (m, 5H), 2.98- 2.90 (m, 4H), 2.87-2.75 (m, 2H), 2.64 (br dd, J = 10.8, 17.2 Hz, 1H), 2.11 (s, 3H), 2.07-1.99 (m, 2H), 1.98-1.87 (m, 3H), 1.85- 1.66 (m, 3H) |
| 407 | 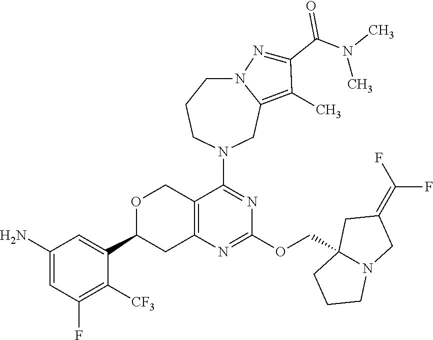 [SMILES: Cc1c(C(=O)N(C)C)nn2c1CN(c1nc(OC[C@@]34CCCN3CC(=C(F)F)C4)nc3c1CO[C@H](c1cc(N)cc(F)c1C(F)(F)F)C3)CCC2] | | A | 721.2 | ¹H NMR (400 MHz, DMSO-d6) δ: 6.73 (d, J = 1.6 Hz, 1H), 6.38 (dd, J = 1.6, 14.8 Hz, 1H), 6.25 (s, 2H), 5.15- 4.92 (m, 2H), 4.85- 4.60 (m, 3H), 4.47- 4.29 (m, 2H), 4.25- 3.98 (m, 2H), 3.97- 3.54 (m, 4H), 3.25 (br dd, J = 2.8, 7.6 Hz, 1H), 3.13-2.89 (m, 7H), 2.85 (br d, J = 15.6 Hz, 1H), 2.76-2.56 (m, 3H), 2.12 (s, 3H), 2.09- 1.79 (m, 6H) |
| 408 | 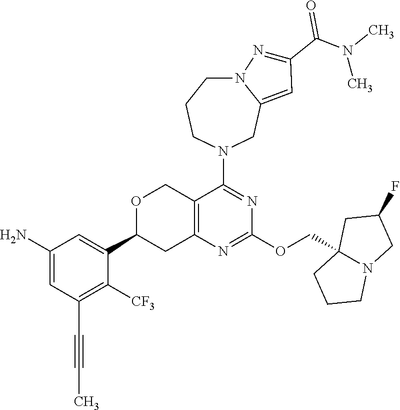 [SMILES: CC#Cc1cc(N)cc([C@@H]2Cc3nc(OC[C@@]45CCCN4C[C@H](F)C5)nc(N4CCCn5nc(C(=O)N(C)C)cc5C4)c3CO2)c1C(F)(F)F] | | A | 697.6 | ¹H NMR (400 MHz, DMSO-d6) δ: 6.86 (d, J = 2.0 Hz, 1H), 6.65 (d, J = 2.0 Hz, 1H), 6.57 (s, 1H), 5.93 (s, 2H), 5.33-5.15 (m, 1H), 5.04-4.92 (m, 2H), 4.72-4.65 (m, 3H), 4.48-4.40 (m, 2H), 3.94-3.88 (m, 1H), 3.87-3.80 (m, 3H), 3.25 (s, 3H), 3.09-3.03 (m, 2H), 2.96 (br d, J = 3.2 Hz, 1H), 2.93 (s, 3H), 2.84-2.76 (m, 2H), 2.61 (br dd, J = 10.8, 18.0 Hz, 1H), 2.17-2.08 (m, 1H), 2.07-2.02 (m, 4H), 1.97 (br d, J = 2.0 Hz, 2H), 1.93 (br d, J = 3.6 Hz, 1H), 1.84-1.78 (m, 1H), 1.75-1.69 (m, 2H) |
| 409 | 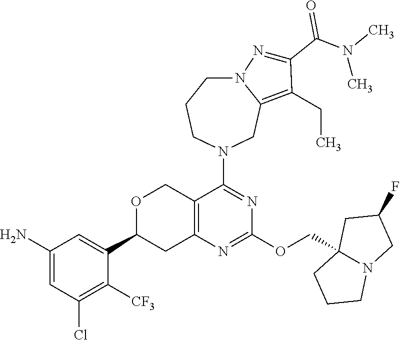 [SMILES: CCc1c(C(=O)N(C)C)nn2c1CN(c1nc(OC[C@@]34CCCN3C[C@H](F)C4)nc3c1CO[C@H](c1cc(N)cc(Cl)c1C(F)(F)F)C3)CCC2] | | A | 721.4 | ¹H NMR (400 MHz, DMSO-d6) δ: 6.88 (d, J = 2.0 Hz, 1H), 6.69 (d, J = 2.0 Hz, 1H), 6.19 (s, 2H), 5.45-5.10 (m, 1H), 5.08-4.96 (m, 2H), 4.93-4.82 (m, 1H), 4.75-4.66 (m, 1H), 4.66-4.56 (m, 1H), 4.44-4.31 (m, 2H), 3.94-3.79 (m, 3H), 3.78-3.66 (m, 1H), 3.08-3.01 (m, 5H), 3.00-2.93 (m, 4H), 2.87-2.78 (m, 2H), 2.67-2.56 (m, 3H), 2.12-2.03 (m, 2H), 2.02-1.96 (m, 2H), 1.94-1.88 (m, 1H), 1.86-1.79 (m, 1H), 1.77-1.69 (m, 2H), 1.01 (t, J = 7.6 Hz, 3H) |
| 450 | 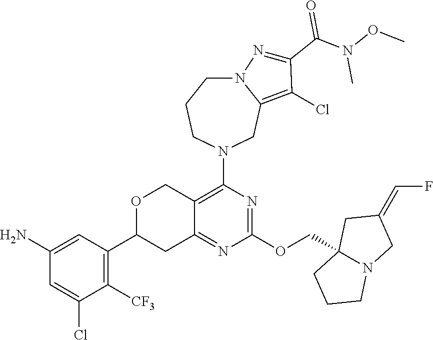 [SMILES: CON(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2] | | A | 735.3 | ¹H NMR (400 MHz, MeOD) δ 6.99 (s, 0.5H), 6.96 (d, J = 2.1 Hz, 1H), 6.77 (d, J = 11.5 Hz, 0.5H), 6.73 (d, J = 2.2 Hz, 1H), 5.15 (d, J = 8.2 Hz, 1H), 5.05 (d, J = 13.7 Hz, 1H), 4.82 (d, J = 4.2 Hz, 4H), 4.50 (d, J = 21.0 Hz, 2H), 4.33 (m, 2H), 4.12 (d, J = 14.8 Hz, 1H), 3.97 (d, J = 14.0 Hz, 1H), 3.85 (d, J = 8.0 Hz, 1H), 3.74 (d, J = 18.1 Hz, 4H), 3.40 (s, 3H), 3.27 (d, J = 10.8 Hz, 1H), 2.96 (dd, J = 51.2, 17.1 Hz, 2H), 2.77 (t, J = 12.8 Hz, 2H), 2.27 (m, 6H), 2.14 (dd, J = 19.1, 12.1 Hz, 3H). |
| 451 | 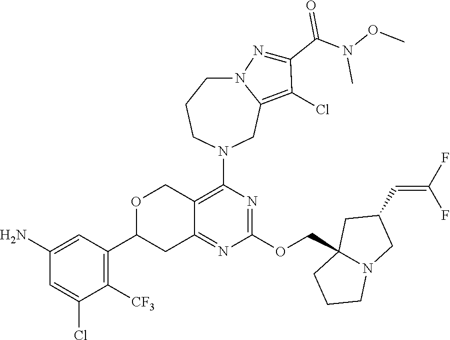 [SMILES: CON(C)C(=O)c1nn2c(c1Cl)CN(c1nc(OC[C@]34CCCN3C[C@@H](C=C(F)F)C4)nc3c1COC(c1cc(N)cc(Cl)c1C(F)(F)F)C3)CCC2] | | A | 787.1 | ¹H NMR (400 MHz, MeOD) δ 6.97 (d, J = 2.2 Hz, 1H), 6.73 (d, J = 2.2 Hz, 1H), 5.18 (d, J = 11.1 Hz, 1H), 5.07 (d, J = 13.9 Hz, 1H), 4.99 (dd, J = 16.3, 4.4 Hz, 1H), 4.90-4.86 (m, 2H), 4.67 (dd, J = 16.3, 8.6 Hz, 1H), 4.57-4.15 (m, 5H), 3.95 (dd, J = 8.7, 4.4 Hz, 1H), 3.90-3.76 (m, 2H), 3.75 (s, 3H), 3.54-3.45 (m, 1H), 3.42-3.34 (m, 4H), 3.07- 2.92 (m, 2H), 2.75 (dd, J = 18.3, 10.6 Hz, 1H), 2.51 (ddd, J = 19.3, 12.8, 6.9 Hz, 1H), 2.43- 2.27 (m, 2H), 2.20 (tt, J = 11.2, 5.6 Hz, 3H), 2.12-2.02 (m, 1H), 1.92 (dt, J = 10.6, 4.3 Hz, 1H). |
| 452 | 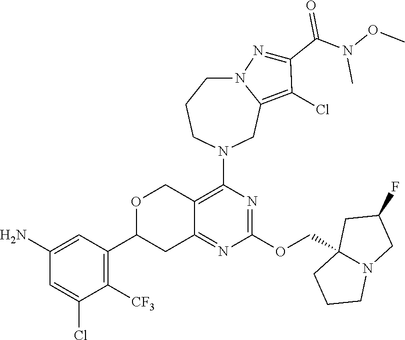 [SMILES: CON(C)C(=O)c1nn2c(c1Cl)CN(c1nc(OC[C@@]34CCCN3C[C@H](F)C4)nc3c1COC(c1cc(N)cc(Cl)c1C(F)(F)F)C3)CCC2] | | A | 743.2 | ¹H NMR (400 MHz, MeOD) δ 6.97 (d, J = 2.3 Hz, 1H), 6.74 (d, J = 2.3 Hz, 1H), 5.66- 5.47 (m, 1H), 5.17 (d, J = 9.1 Hz, 1H), 5.10-4.95 (m, 2H), 4.66 (d, J = 16.3 Hz, 1H), 4.53- 4.46 (m, 2H), 4.32-4.18 (m, 2H), 3.99-3.81 (m, 5H), 3.76 (s, 3H), 3.46-3.35 (m, 4H), 3.02 (d, J = 18.0 Hz, 1H), 2.84-2.03 (m, 10H) |
| 453 | 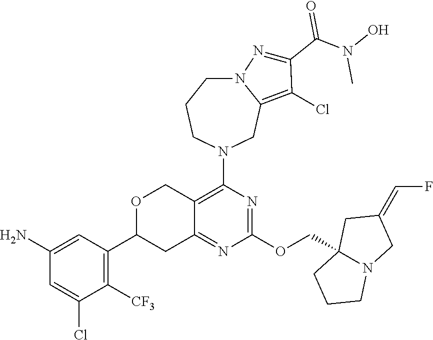 [SMILES: CN(O)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2] | | B | 725.2 | ¹H NMR (400 MHz, MeOD) δ 7.26 (d, J = 8.6 Hz, 1H), 6.98 (d, J = 7.3 Hz, 0.5H), 6.86 (t, J = 8.5 Hz, 1H), 6.77 (d, J = 7.3 Hz, 0.5H), 5.16 (dd, J = 11.2, 3.8 Hz, 1H), 5.03-4.94 (m, 2H), 4.79 (d, J = 13.9 Hz, 1H), 4.64 (d, J = 16.2 Hz, 1H), 4.55-4.43 (m, 2H), 4.40 (d, J = 14.4 Hz, 1H), 4.29 (dd, J = 16.8, 12.1 Hz, 1H), 4.23- 4.16 (m, 1H), 4.11 (d, J = 15.6 Hz, 1H), 3.95 (dd, J = 9.5, 4.4 Hz, 1H), 3.76 (ddd, J = 17.6, 16.5, 8.0 Hz, 2H), 3.40 (s, 3H), 3.32 (s, 1H), 3.25 (dd, J = 12.8, 6.3 Hz, 1H), 3.05-2.86 (m, 2H), 2.81-2.70 (m, 1H), 2.44- 2.30 (m, 2H), 2.27- 2.05 (m, 4H) |
| 454 | 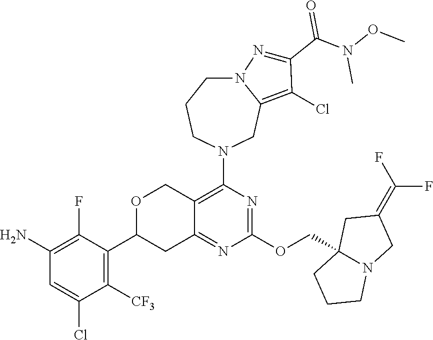 [SMILES: CON(C)C(=O)c1nn2c(c1Cl)CN(c1nc(OC[C@@]34CCCN3CC(=C(F)F)C4)nc3c1COC(c1c(F)c(N)cc(Cl)c1C(F)(F)F)C3)CCC2] | | A | 791.1 | ¹H NMR (400 MHz, MeOD) δ 6.92 (d, J = 7.9 Hz, 1H), 5.26 (dd, J = 11.2, 4.0 Hz, 1H), 4.97 (dd, J = 15.1, 6.1 Hz, 2H), 4.77 (d, J = 13.8 Hz, 1H), 4.62 (d, J = 16.4 Hz, 1H), 4.51- 4.36 (m, 4H), 4.22 (dd, J = 14.3, 8.8 Hz, 1H), 4.08 (d, J = 14.3 Hz, 1H), 3.93 (dt, J = 13.4, 4.3 Hz, 1H), 3.82-3.72 (m, 5H), 3.37 (s, 4H), 3.27-3.20 (m, 1H), 2.86 (dd, J = 44.9, 17.3 Hz, 3H), 2.37 (dd, J = 12.4, 6.8 Hz, 2H), 2.28- 2.12 (m, 4H) |
| 455 | 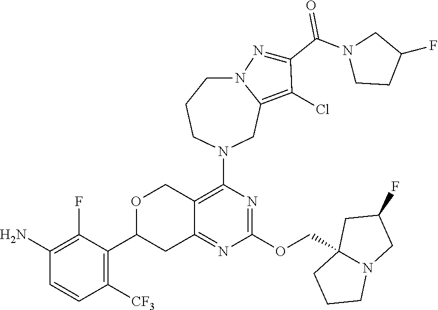 [SMILES: Nc1ccc(C(F)(F)F)c(C2Cc3nc(OC[C@@]45CCCN4C[C@H](F)C5)nc(N4CCCn5nc(C(=O)N6CCC(F)C6)c(Cl)c5C4)c3CO2)c1F] | | A | 805.2 | ¹H NMR (400 MHz, MeOD) δ 7.20 (d, J = 8.6 Hz, 1H), 6.79 (t, J = 8.5 Hz, 1H), 5.22 (d, J = 55.0 Hz, 1H), 5.12-5.06 (m, 1H), 4.97 (d, J = 13.7 Hz, 1H), 4.78- 4.73 (m, 1H), 4.67 (dd, J = 15.2, 4.1 Hz, 2H), 4.44 (dt, J = 13.9, 6.9 Hz, 1H), 4.26 (dd, J = 16.7, 7.5 Hz, 1H), 4.02 (ddd, J = 12.2, 9.3, 3.8 Hz, 2H), 3.89 (dt, J = 12.8, 9.3 Hz, 3H), 3.83- 3.56 (m, 4H), 3.22- 3.12 (m, 3H), 3.07 (s, 1H), 2.96- 2.89 (m, 1H), 2.79 (dd, J = 17.8, 3.7 Hz, 1H), 2.29 (d, J = 10.7 Hz, 1H), 2.23-2.17 (m, 1H), 2.15-1.98 (m, 5H), 1.92- 1.80 (m, 3H) |
| 463 | 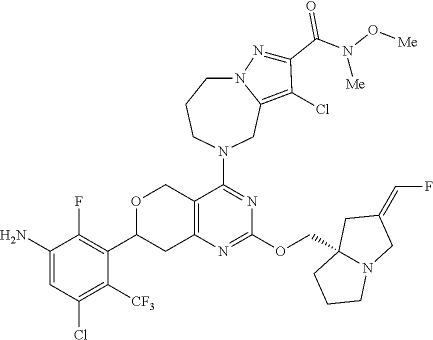 [SMILES: CON(C)C(=O)c1nn2c(c1Cl)CN(c1nc(OC[C@@]34CCCN3C/C(=C\F)C4)nc3c1COC(c1c(F)c(N)cc(Cl)c1C(F)(F)F)C3)CCC2] | | A | 773.2 | ¹H NMR (400 MHz, MeOD) δ 6.99 (d, J = 7.3 Hz, 1H), 6.92 (d, J = 7.9 Hz, 1H), 6.78 (d, J = 7.3 Hz, 1H), 5.26 (dd, J = 11.1, 4.4 Hz, 1H), 4.98 (dd, J = 16.1, 2.5 Hz, 2H), 4.78 (d, J = 13.9 Hz, 1H), 4.64 (d, J = 16.4 Hz, 1H), 4.56-4.44 (m, 2H), 4.42-4.16 (m, 3H), 4.12 (d, J = 15.5 Hz, 1H), 3.99-3.91 (m, 1H), 3.84-3.70 (m, 5H), 3.38 (s, 3H), 3.26 (dd, J = 12.0, 7.8 Hz, 2H), 3.05-2.88 (m, 2H), 2.76 (dd, J = 22.1, 16.0 Hz, 1H), 2.46-2.31 (m, 2H), 2.16 (dddd, J = 22.9, 19.4, 14.2, 6.8 Hz, 4H) |
| 464 | 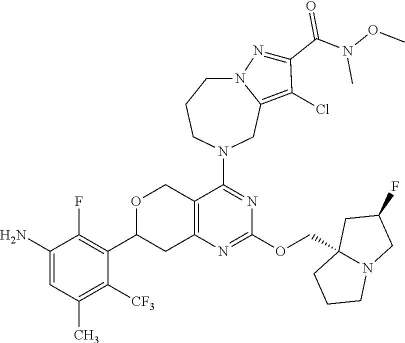 [SMILES: CON(C)C(=O)c1nn2c(c1Cl)CN(c1nc(OC[C@@]34CCCN3C[C@H](F)C4)nc3c1COC(c1c(F)c(N)cc(C)c1C(F)(F)F)C3)CCC2] | | A | 741.1 | ¹H NMR (400 MHz, MeOD) δ 6.70 (d, J = 8.5 Hz, 1H), 5.63 (s, 0.5H), 5.50 (s, 0.5H), 5.22 (d, J = 6.9 Hz, 1H), 4.98 (t, J = 13.4 Hz, 2H), 4.77 (d, J = 13.7 Hz, 2H), 4.61 (d, J = 17.3 Hz, 1H), 4.53-4.45 (m, 2H), 4.21 (d, J = 9.3 Hz, 2H), 4.01- 3.89 (m, 2H), 3.85 (d, J = 14.4 Hz, 3H), 3.75 (s, 3H), 3.45-3.36 (m, 4H), 2.89 (d, J = 17.1 Hz, 1H), 2.74-2.52 (m, 2H), 2.46-2.28 (m, 7H), 2.17 (s, 2H) |
| 465 | 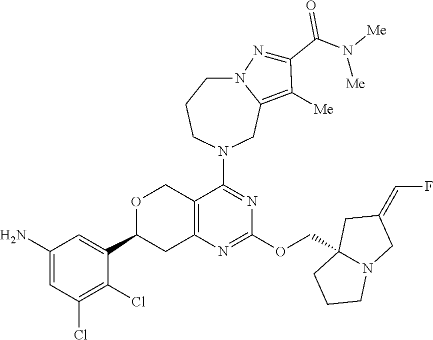 [SMILES: Cc1c(C(=O)N(C)C)nn2c1CN(c1nc(OC[C@@]34CCCN3C/C(=C\F)C4)nc3c1CO[C@H](c1cc(N)cc(Cl)c1Cl)C3)CCC2] | | N.D. | 685.2 | ¹H NMR (400 MHz, CDCl₃) δ 6.7- 6.8 (m, 2H) 6.4- 6.6 (m, 1H) 5.0 (dd, J = 11.2, 3.6 Hz, 1H) 4.9-5.0 (m, 2H) 4.7 (d, J = 13.6 Hz, 1H) 4.4- 4.5 (m, 1H) 4.3- 4.4 (m, 2H) 4.0 (s, 2H) 3.7-3.9 (m, 4H) 3.6-3.7 (m, 1H) 3.4 (br d, J = 14.8 Hz, 1H) 3.2- 3.3 (m, 1H) 3.1- 3.2 (m, 4H) 3.1 (br s, 3H) 2.6-2.8 (m, 3H) 2.3 (br d, J = 15.6 Hz, 1H) 2.2 (s, 4H) 2.1 (br d, J = 4.8 Hz, 2H) 1.8- 1.9 (m, 2H) 1.8 (br dd, J = 12.4, 8.4 Hz, 1H) |
| 466 | 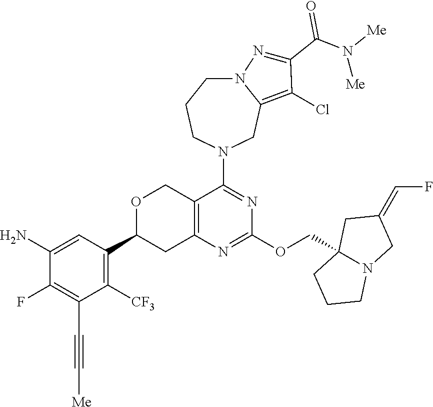 [SMILES: CC#Cc1c(F)c(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F] | | N.D. | 761.4 | ¹H NMR (400 MHz, CDCl₃) δ 6.7- 6.8 (m, 2H) 6.4- 6.6 (m, 1H) 5.0 (dd, J = 11.2, 3.6 Hz, 1H) 4.9-5.0 (m, 2H) 4.7 (d, J = 13.6 Hz, 1H) 4.4- 4.5 (m, 1H) 4.3- 4.4 (m, 2H) 4.0 (s, 2H) 3.7-3.9 (m, 4H) 3.6-3.7 (m, 1H) 3.4 (br d, J = 14.8 Hz, 1H) 3.2- 3.3 (m, 1H) 3.1- 3.2 (m, 4H) 3.1 (br s, 3H) 2.6-2.8 (m, 3H) 2.3 (br d, J = 15.6 Hz, 1H) 2.2 (s, 4H) 2.1 (br d, J = 4.8 Hz, 2H) 1.8- 1.9 (m, 2H) 1.8 (br dd, J = 12.4, 8.4 Hz, 1H) |

pERK IC₅₀ activity ranges: A = 10 to 30 nM; B = 31-100 nM; C = 101-500 nM; D = >500 nM

TABLE 1b

| | | PERK | | |
|---|---|---|---|---|
| | | IC₅₀ | | |
| | | (AsPC-1, | MS | |
| Ex. | Structure | 4h) nM | (ESI) | ¹H-NMR |
| 213a | 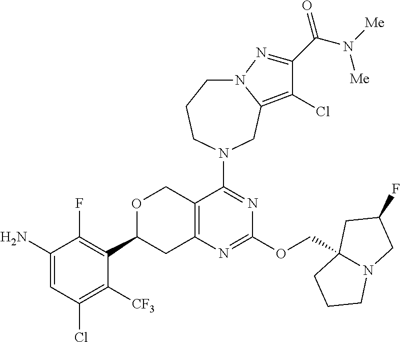 [SMILES: CN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2] | <10 | 745.2 | ¹H NMR (400 MHz, CDCl₃) δ 6.84 (d, J = 8.0 Hz, 1H), 5.35-5.21 (m, 1H), 5.20-5.19 (m, 2H), 4.96 (br d, J = 13.2 Hz, 1H), 4.81 (br d, J = 16.4 Hz, 1H), 4.69 (d, J = 13.6 Hz, 1H), 4.55-4.40 (m, 1H), 4.38-4.31 (m, 1H), 4.18 (s, 2H), 4.03 (s, 2H), 3.96-3.89 (m, 1H), 3.68-3.59 (m, 1H), 3.37- 3.14 (m, 4H), 3.10 (s, 6H), 3.02-2.94 (m, 2H), 2.24 (br d, J = 1.6 Hz, 2H), 2.20-2.10 (m, 3H), 1.99-1.84 (m, 3H) |
| 214a | 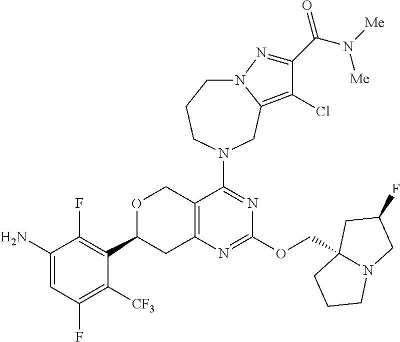 [SMILES: CN(C)C(=O)c1nn2c(c1Cl)CN(c1nc(OC[C@@]34CCCN3C[C@H](F)C4)nc3c1CO[C@H](c1c(F)c(N)cc(F)c1C(F)(F)F)C3)CCC2] | <10 | 729.3 | ¹H NMR (400 MHz, CDCl₃) δ 6.50 (dd, J = 7.2, 12.4 Hz, 1H), 5.36- 5.17 (m, 1H), 5.17-5.10 (m, 1H), 4.97 (br d, J = 13.6 Hz, 1H), 4.81 (d, J = 16.4 Hz, 1H), 4.70 (d, J = 13.6 Hz, 1H), 4.56-4.40 (m, 2H), 4.34 (br dd, J = 10.4, 12.8 Hz, 1H), 4.27 (s, 2H), 4.02 (s, 2H), 3.97-3.86 (m, 1H), 3.64 (ddd, J = 3.2, 10.4, 13.7 Hz, 1H), 3.39-3.16 (m, 3H), 3.10 (s, 6H), 3.01- 2.91 (m, 2H), 2.37-2.07 (m, 5H), 1.98-1.79 (m, 3H) |
| 215a | 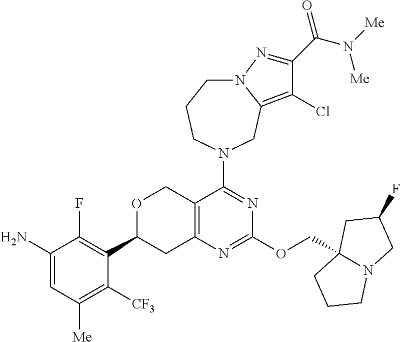 [SMILES: Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F] | <10 | 725.3 | ¹H NMR (400 MHz, CDCl₃) δ 6.8 (d, J = 8.4 Hz, 1H), 5.37-5.13 (m, 2H), 4.97 (br d, J = 13.6 Hz, 1H), 4.81 (br d, J = 16.4 Hz, 1H), 4.69 (d, J = 13.6 Hz, 1H), 4.58-4.38 (m, 2H), 4.34 (br dd, J = 10.4, 12.8 Hz, 1H), 4.02 (br d, J = 14.8 Hz, 4H), 3.95-3.83 (m, 1H), 3.63 (ddd, J = 3.2, 10.4, 14.0 Hz, 1H), 3.36 (dd, J = 12.0, 18.0 Hz, 1H), 3.31- 3.17 (m, 2H), 3.15 (br s, 1H), 3.10 (s, 6H), 3.01- 2.93 (m, 2H), 2.39 (q, J = 4.0 Hz, 3H), 2.34- 2.21 (m, 2H), 2.20-2.04 (m, 3H), 1.98-1.82 (m, 3H). |
| 234 | 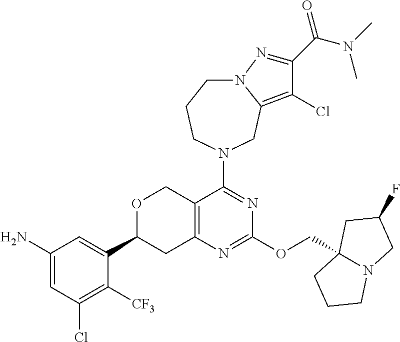 [SMILES: CN(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2] | <10 | 727.3 | ¹H NMR (400 MHz, DMSO-d₆) δ: 6.91-6.84 (m, 1H), 6.70 (d, J = 2.0 Hz, 1H), 6.26-6.15 (m, 2H), 5.33-5.13 (m, 1H), 5.10-4.98 (m, 2H), 4.88- 4.76 (m, 1H), 4.75- 4.64 (m, 2H), 4.47-4.27 (m, 2H), 3.91-3.79 (m, 3H), 3.78-3.66 (m, 1H), 3.08-3.01 (m, 2H), 2.99 (s, 3H), 2.96 (s, 3H), 2.89-2.79 (m, 2H), 2.68- 2.57 (m, 1H), 2.36- 2.11 (m, 2H), 2.10-2.00 (m, 2H), 1.98-1.89 (m, 2H), 1.85-1.77 (m, 1H), 1.75-1.62 (m, 2H) |
| 235 | 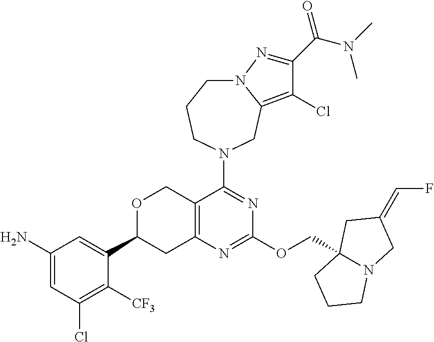 [SMILES: CN(C)C(=O)c1nn2c(c1Cl)CN(c1nc(OC[C@@]34CCCN3C/C(=C\F)C4)nc3c1CO[C@H](c1cc(N)cc(Cl)c1C(F)(F)F)C3)CCC2] | <10 | 739.3 | ¹H NMR (400 MHz, DMSO-d₆) δ: 6.88 (s, 1H), 6.84 (br s, 1H), 6.70 (d, J = 2.0 Hz, 1H), 6.22 (s, 2H), 5.10-5.00 (m, 2H), 4.88 (br d, J = 16.8 Hz, 1H), 4.74-4.63 (m, 2H), 4.48-4.35 (m, 2H), 3.95-3.88 (m, 1H), 3.88- 3.81 (m, 2H), 3.78- 3.68 (m, 1H), 3.65 (br d, J = 14.8 Hz, 1H), 2.98 (s, 3H), 2.96 (s, 3H), 2.90- 2.82 (m, 1H), 2.70-2.59 (m, 1H), 2.45 (s, 1H), 2.35-2.22 (m, 2H), 2.22- 2.14 (m, 1H), 2.11- 1.94 (m, 2H), 1.93-1.76 (m, 3H), 1.76-1.64 (m, 2H) |
| 236 | 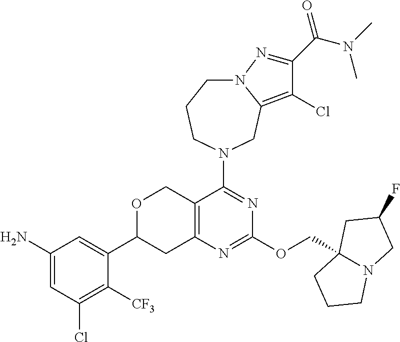 [SMILES: CN(C)C(=O)c1nn2c(c1Cl)CN(c1nc(OC[C@@]34CCCN3C[C@H](F)C4)nc3c1COC(c1cc(N)cc(Cl)c1C(F)(F)F)C3)CCC2] | <10 | 727.1 | ¹H NMR (400 MHz, DMSO-d₆) δ: 6.88 (s, 1H), 6.70 (d, J = 2.0 Hz, 1H), 6.22 (s, 2H), 5.35- 5.14 (m, 1H), 5.09-5.00 (m, 2H), 4.88-4.77 (m, 1H), 4.75-4.64 (m, 2H), 4.48-4.30 (m, 2H), 3.95 (d, J = 10.8 Hz, 1H), 3.89-3.80 (m, 2H), 3.79- 3.67 (m, 1H), 3.10- 3.02 (m, 2H), 2.99 (s, 3H), 2.96 (s, 3H), 2.91- 2.79 (m, 2H), 2.69-2.55 (m, 1H), 2.46-2.13 (m, 2H), 2.11-2.01 (m, 2H), 2.00-1.95 (m, 1H), 1.94- 1.87 (m, 1H), 1.85- 1.78 (m, 1H), 1.77-1.68 (m, 2H) |
| 240 | 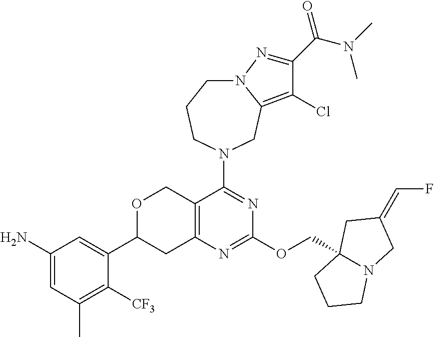 [SMILES: Cc1cc(N)cc(C2Cc3nc(OC[C@@]45CCCN4C/C(=C\F)C5)nc(N4CCCn5nc(C(=O)N(C)C)c(Cl)c5C4)c3CO2)c1C(F)(F)F] | <10 | 719.4 | ¹H NMR (400 MHz, DMSO-d₆) δ: 7.19-6.76 (m, 1H), 6.74 (s, 1H), 6.42 (s, 1H), 5.78 (s, 2H), 5.11-4.92 (m, 2H), 4.87-4.68 (m, 3H), 4.51- 4.26 (m, 3H), 4.25- 4.02 (m, 2H), 3.94-3.66 (m, 3H), 3.44-3.36 (m, 1H), 2.97 (d, J = 12.0 Hz, 6H), 2.88-2.80 (m, 1H), 2.74-2.64 (m, 2H), 2.59-2.53 (m, 1H), 2.48- 2.44 (m, 1H), 2.34- 2.27 (m, 3H), 2.24-2.16 (m, 1H), 2.12-1.82 (m, 5H) |
| 242 | 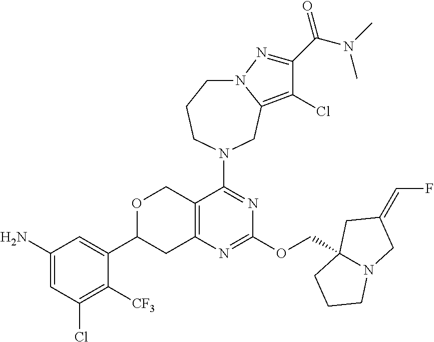 [SMILES: CN(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2] | <10 | 739.3 | ¹H NMR (400 MHz, DMSO-d₆) δ: 6.88 (s, 1H), 6.84 (br s, 1H), 6.70 (d, J = 2.0 Hz, 1H), 6.25- 6.17 (m, 2H), 5.09- 5.00 (m, 2H), 4.91-4.82 (m, 1H), 4.75-4.64 (m, 2H), 4.47-4.33 (m, 2H), 3.97-3.78 (m, 4H), 3.77- 3.62 (m, 3H), 2.98 (s, 3H), 2.96 (s, 3H), 2.91- 2.68 (m, 2H), 2.68-2.54 (m, 2H), 2.30-2.14 (m, 2H), 2.12-1.98 (m, 1H), 1.93-1.81 (m, 2H), 1.78- 1.61 (m, 2H) |
| 245 | 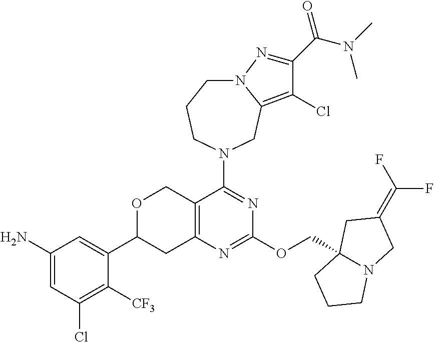 [SMILES: CN(C)C(=O)c1nn2c(c1Cl)CN(c1nc(OC[C@@]34CCCN3CC(=C(F)F)C4)nc3c1COC(c1cc(N)cc(Cl)c1C(F)(F)F)C3)CCC2] | <10 | 757.3 | ¹H NMR (400 MHz, DMSO-d₆) δ: 6.91-6.86 (m, 1H), 6.70 (d, J = 2.0 Hz, 1H), 6.23 (s, 2H), 5.14-5.00 (m, 2H), 4.84- 4.72 (m, 3H), 4.44 (br dd, J = 7.6, 14.4 Hz, 1H), 4.39-4.27 (m, 2H), 4.24 (br d, J = 8.8 Hz, 2H), 3.94-3.71 (m, 3H), 3.18- 3.06 (m, 1H), 2.98 (d, J = 12.8 Hz, 7H), 2.88 (br d, J = 15.2 Hz, 1H), 2.81- 2.72 (m, 1H), 2.71- 2.60 (m, 2H), 2.27-2.18 (m, 1H), 2.13-1.80 (m, 5H) |
| 247a | 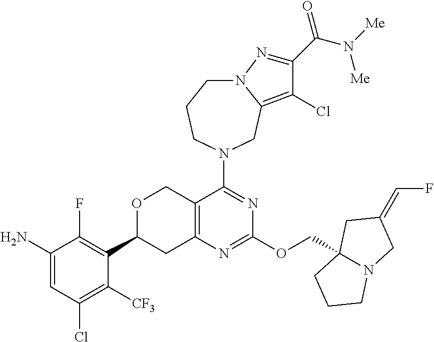 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2] | <10 | 757.2 | ¹H NMR (400 MHz, CDCl3) δ 6.84 (d, J = 8.0 Hz, 1H), 6.62-6.37 (m, 1H), 5.21 (dd, J = 4.2, 11.6 Hz, 1H), 4.99 (br d, J = 13.2 Hz, 1H), 4.84 (d, J = 16.8 Hz, 1H), 4.69 (d, J = 13.6 Hz, 1H), 4.55- 4.47 (m, 1H), 4.45-4.33 (m, 2H), 4.18 (s, 2H), 4.08-3.98 (m, 2H), 3.97- 3.91 (m, 1H), 3.83 (br d, J = 15.2 Hz, 1H), 3.72- 3.62 (m, 1H), 3.43- 3.28 (m, 2H), 3.18-3.12 (m, 1H), 3.10 (s, 6H), 2.98 (br dd, J = 4.0, 18.0 Hz, 1H), 2.71-2.59 (m, 2H), 2.36-2.27 (m, 2H), 2.13 (ddd, J = 4.8, 7.6, 12.4 Hz, 2H), 1.94-1.87 (m, 2H), 1.79 (br d, J = 8.0 Hz, 1H) |
| 248a | 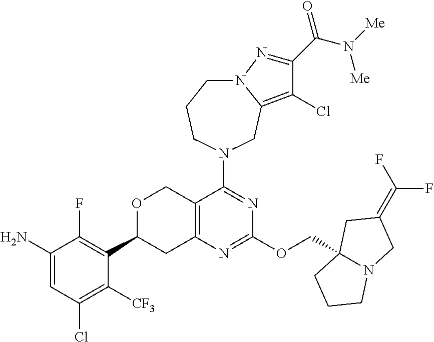 [SMILES: CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2] | <10 | 775.3 | ¹H NMR (400 MHz, CDCl3) δ 6.84 (d, J = 7.6 Hz, 1H), 5.20 (br dd, J = 3.6, 11.2 Hz, 1H), 4.98 (d, J = 13.6 Hz, 1H), 4.84 (d, J = 16.4 Hz, 1H), 4.69 (d, J = 13.6 Hz, 1H), 4.56- 4.47 (m, 1H), 4.43 (d, J = 16.4 Hz, 1H), 4.39- 4.31 (m, 1H), 4.19 (s, 2H), 4.10-4.04 (m, 1H), 4.03-3.97 (m, 1H), 3.97- 3.89 (m, 1H), 3.76- 3.61 (m, 2H), 3.41-3.28 (m, 2H), 3.17-3.12 (m, 1H), 3.10 (s, 6H), 2.98 (br dd, J = 3.6, 18.0 Hz, 1H), 2.76 (br d, J = 15.2 Hz, 1H), 2.67-2.58 (m, 1H), 2.41-2.30 (m, 2H), 2.19-2.11 (m, 2H), 1.96- 1.88 (m, 2H), 1.87 (br s, 1H) |
| 300 | 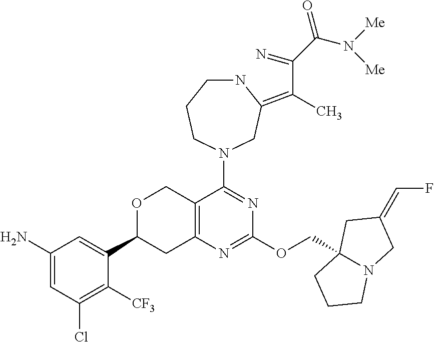 [SMILES: C/C(C(=N)C(=O)N(C)C)=C1\CN(c2nc(OC[C@@]34CCCN3C/C(=C\F)C4)nc3c2CO[C@H](c2cc(N)cc(Cl)c2C(F)(F)F)C3)CCCN1] | <10 | 719.4 | ¹H NMR (400 MHz, DMSO-d₆) δ: 6.90-6.61 (m, 3H), 6.21 (s, 2H), 5.08-4.96 (m, 2H), 4.84- 4.57 (m, 3H), 4.46- 4.31 (m, 2H), 3.89-3.76 (m, 4H), 3.65 (br d, J = 15.2 Hz, 1H), 3.33-3.23 (m, 3H), 3.06 (s, 3H), 2.93 (s, 5H), 2.62 (br dd, J = 10.8, 17.4 Hz, 1H), 2.45 (s, 1H), 2.26 (br d, J = 16.4 Hz, 1H), 2.11 (s, 3H), 2.04-1.95 (m, 1H), 1.91-1.79 (m, 2H), 1.78- 1.61 (m, 2H) |
| 301 | 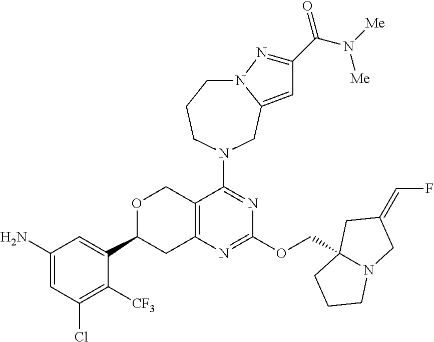 [SMILES: CN(C)C(=O)c1cc2n(n1)CCCN(c1nc(OC[C@@]34CCCN3C/C(=C\F)C4)nc3c1CO[C@H](c1cc(N)cc(Cl)c1C(F)(F)F)C3)C2] | <10 | 705.4 | ¹H NMR (400 MHz, DMSO-d₆) δ: 6.89 (d, J = 2.0 Hz, 1H), 6.84 (br s, 1H), 6.69 (d, J = 2.0 Hz, 1H), 6.62 (s, 1H), 6.57 (s, 1H), 6.20 (s, 2H), 5.04 (br d, J = 12.8 Hz, 2H), 4.73-4.66 (m, 3H), 4.47-4.42 (m, 2H), 3.87- 3.82 (m, 3H), 3.66 (br d, J = 15.6 Hz, 1H), 3.24 (s, 3H), 2.93 (s, 3H), 2.90-2.78 (m, 2H), 2.73- 2.59 (m, 2H), 2.34- 2.24 (m, 2H), 2.18-2.06 (m, 2H), 1.98-1.81 (m, 4H), 1.75-1.63 (m, 2H) |
| 302 | 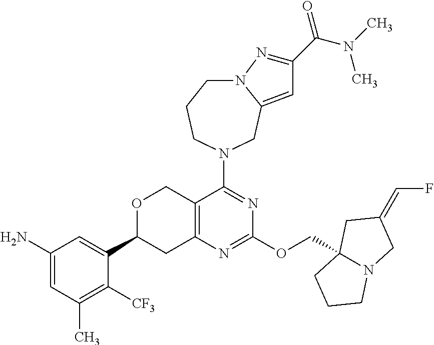 [SMILES: Cc1cc(N)cc([C@@H]2Cc3nc(OC[C@@]45CCCN4C/C(=C\F)C5)nc(N4CCCn5nc(C(=O)N(C)C)cc5C4)c3CO2)c1C(F)(F)F] | <10 | 685.3 | ¹H NMR (400 MHz, DMSO-d₆) δ: 6.90-6.64 (m, 1H), 6.57 (s, 1H), 6.41 (s, 1H), 5.76 (s, 2H), 5.06-4.92 (m, 2H), 4.78-4.65 (m, 3H), 4.62- 4.37 (m, 3H), 3.99- 3.80 (m, 4H), 3.52-3.39 (m, 3H), 3.24 (s, 3H), 2.93 (s, 3H), 2.84-2.78 (m, 1H), 2.68-2.64 (m, 1H), 2.63-2.60 (m, 1H), 2.32-2.28 (m, 3H), 2.20- 1.67 (m, 8H) |
| 410 | 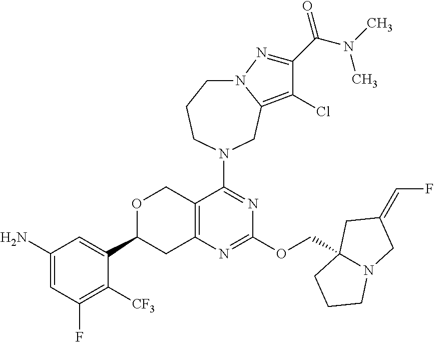 [SMILES: CN(C)C(=O)c1nn2c(c1Cl)CN(c1nc(OC[C@@]34CCCN3C/C(=C\F)C4)nc3c1CO[C@H](c1cc(N)cc(F)c1C(F)(F)F)C3)CCC2] | <10 | 723.5 | ¹H NMR (400 MHz, DMSO-d₆) δ: 6.93-6.65 (m, 2H), 6.39 (d, J = 14.4 Hz, 1H), 6.25 (s, 2H), 5.10-4.93 (m, 2H), 4.89- 4.79 (m, 1H), 4.76- 4.64 (m, 2H), 4.48-4.31 (m, 2H), 4.11-3.91 (m, 2H), 3.88-3.68 (m, 3H), 3.18-3.07 (m, 1H), 2.97 (d, J = 9.6 Hz, 7H), 2.89- 2.81 (m, 1H), 2.74- 2.61 (m, 2H), 2.44-2.29 (m, 1H), 2.26-2.14 (m, 1H), 2.13-1.64 (m, 6H) |
| 411 | 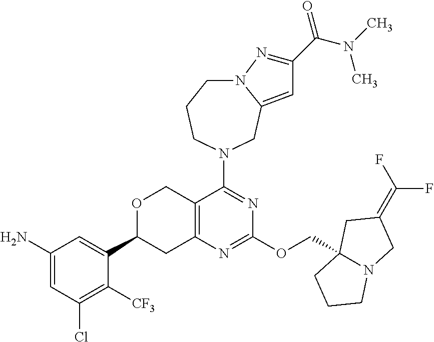 [SMILES: CN(C)C(=O)c1cc2n(n1)CCCN(c1nc(OC[C@@]34CCCN3CC(=C(F)F)C4)nc3c1CO[C@H](c1cc(N)cc(Cl)c1C(F)(F)F)C3)C2] | <10 | 723.3 | ¹H NMR (400 MHz, DMSO-d₆) δ: 6.90 (s, 1H), 6.70 (s, 1H), 6.58 (s, 1H), 6.23 (s, 2H), 5.10 (s, 2H), 4.62-4.76 (m, 3H), 4.38-4.50 (m, 2H), 3.94 (s, 2H), 3.80- 3.88 (m, 2H), 3.54-3.66 (m, 1H), 3.26-3.28 (m, 1H), 3.25 (s, 3H), 2.97- 3.03 (m, 1H), 2.94 (s, 3H), 2.79-2.87 (m, 1H), 2.61-2.69 (m, 1H), 2.53- 2.60 (m, 1H), 2.27- 2.43 (m, 2H), 2.09-2.18 (m, 1H), 1.69-1.96 (m, 5H) |
| 412 | 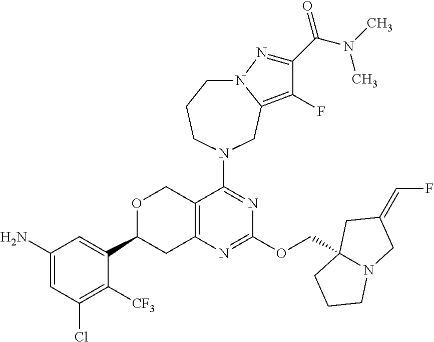 [SMILES: CN(C)C(=O)c1nn2c(c1F)CN(c1nc(OC[C@@]34CCCN3C/C(=C\F)C4)nc3c1CO[C@H](c1cc(N)cc(Cl)c1C(F)(F)F)C3)CCC2] | <10 | 723.3 | ¹H NMR (400 MHz, DMSO-d₆) δ: 6.89 (d, J = 2.0 Hz, 1H), 6.86-6.60 (m, 2H), 6.22 (s, 2H), 5.06-4.94 (m, 2H), 4.80- 4.68 (m, 3H), 4.46- 4.34 (m, 2H), 3.93-3.76 (m, 4H), 3.65 (br d, J = 14.4 Hz, 1H), 3.28 (br d, J = 15.6 Hz, 1H), 3.10 (s, 3H), 3.01-2.92 (m, 4H), 2.88-2.79 (m, 1H), 2.70- 2.59 (m, 1H), 2.45 (s, 1H), 2.27 (br d, J = 15.6 Hz, 1H), 2.17-2.05 (m, 1H), 2.05-1.78 (m, 4H), 1.78-1.62 (m, 2H) |
| 413 | 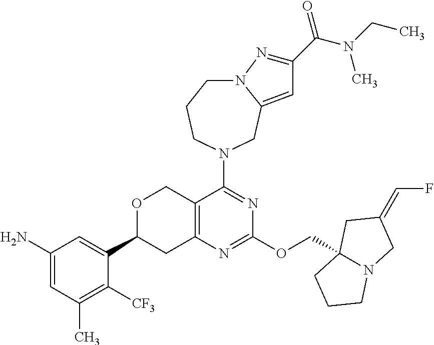 [SMILES: CCN(C)C(=O)c1cc2n(n1)CCCN(c1nc(OC[C@@]34CCCN3C/C(=C\F)C4)nc3c1CO[C@H](c1cc(N)cc(C)c1C(F)(F)F)C3)C2] | <10 | 699.6 | ¹H NMR (400 MHz, DMSO-d₆) δ: 7.05 (d, J = 81.6 Hz, 1H), 6.76 (s, 1H), 6.63 (d, J = 9.6 Hz, 1H), 6.43 (s, 1H), 5.77 (s, 2H), 5.08-4.95 (m, 2H), 4.84-4.64 (m, 3H), 4.54-4.12 (m, 5H), 4.09- 3.77 (m, 3H), 3.72- 3.63 (m, 1H), 3.43-3.38 (m, 2H), 3.24 (s, 2H), 2.99-2.88 (m, 2H), 2.87- 2.74 (m, 2H), 2.70- 2.60 (m, 2H), 2.34-2.28 (m, 3H), 2.20-1.92 (m, 6H), 1.16-1.06 (m, 3H) |
| 414 | 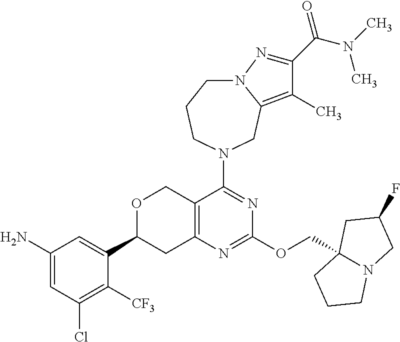 [SMILES: Cc1c(C(=O)N(C)C)nn2c1CN(c1nc(OC[C@@]34CCCN3C[C@H](F)C4)nc3c1CO[C@H](c1cc(N)cc(Cl)c1C(F)(F)F)C3)CCC2] | <10 | 707.4 | ¹H NMR (400 MHz, DMSO-d₆) δ: 6.88 (d, J = 2.0 Hz, 1H), 6.69 (d, J = 2.0 Hz, 1H), 6.20 (s, 2H), 5.35-5.12 (m, 1H), 5.08- 4.91 (m, 2H), 4.79- 4.63 (m, 3H), 4.44-4.29 (m, 2H), 3.89-3.74 (m, 4H), 3.10-3.02 (m, 5H), 2.94 (s, 3H), 2.87-2.77 (m, 2H), 2.69-2.59 (m, 1H), 2.12 (s, 3H), 2.08- 1.97 (m, 3H), 1.93-1.66 (m, 5H) |
| 415 | 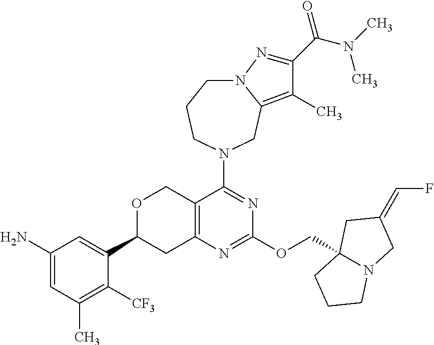 [SMILES: Cc1cc(N)cc([C@@H]2Cc3nc(OC[C@@]45CCCN4C/C(=C\F)C5)nc(N4CCCn5nc(C(=O)N(C)C)c(C)c5C4)c3CO2)c1C(F)(F)F] | <10 | 699.3 | ¹H NMR (400 MHz, DMSO-d₆) δ: 6.88-6.58 (m, 2H), 6.41 (s, 1H), 5.75 (s, 2H), 5.05-4.89 (m, 2H), 4.85-4.74 (m, 1H), 4.73-4.57 (m, 2H), 4.47-4.27 (m, 2H), 3.90- 3.72 (m, 4H), 3.69- 3.59 (m, 1H), 3.29-3.24 (m, 1H), 3.06 (s, 3H), 3.00-2.95 (m, 1H), 2.93 (s, 3H), 2.78 (br d, J = 1.6 Hz, 1H), 2.69-2.58 (m, 1H), 2.46-2.44 (m, 1H), 2.33-2.24 (m, 4H), 2.10 (s, 3H), 2.08-2.00 (m, 1H), 1.98-1.77 (m, 4H), 1.76-1.60 (m, 2H) |
| 416 | 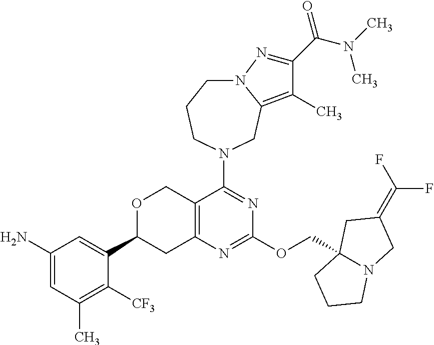 [SMILES: Cc1cc(N)cc([C@@H]2Cc3nc(OC[C@@]45CCCN4CC(=C(F)F)C5)nc(N4CCCn5nc(C(=O)N(C)C)c(C)c5C4)c3CO2)c1C(F)(F)F] | <10 | 716.7 | ¹H NMR (400 MHz, DMSO-d₆) δ: 6.74 (s, 1H), 6.41 (s, 1H), 5.76 (s, 2H), 5.07-4.90 (m, 2H), 4.80-4.61 (m, 3H), 4.46-4.29 (m, 2H), 3.96- 3.88 (m, 2H), 3.84- 3.77 (m, 2H), 3.66-3.51 (m, 1H), 3.32-3.21 (m, 3H), 3.05 (s, 3H), 2.93 (s, 3H), 2.85-2.77 (m, 1H), 2.66-2.54 (m, 2H), 2.36-2.28 (m, 4H), 2.11 (s, 3H), 1.97-1.66 (m, 6H) |
| 417 | 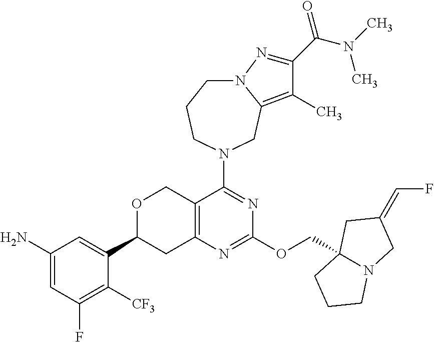 [SMILES: Cc1c(C(=O)N(C)C)nn2c1CN(c1nc(OC[C@@]34CCCN3C/C(=C\F)C4)nc3c1CO[C@H](c1cc(N)cc(F)c1C(F)(F)F)C3)CCC2] | <10 | 703.5 | ¹H NMR (400 MHz, DMSO-d₆) δ: 6.89-6.61 (m, 2H), 6.38 (dd, J = 2.0, 14.8 Hz, 1H), 6.24 (s, 2H), 5.04-4.91 (m, 2H), 4.83-4.58 (m, 3H), 4.46-4.27 (m, 2H), 3.93- 3.84 (m, 2H), 3.83- 3.59 (m, 4H), 3.15-2.97 (m, 4H), 2.93 (s, 3H), 2.83 (br dd, J = 2.8, 17.6 Hz, 1H), 2.72-2.55 (m, 2H), 2.33-2.24 (m, 1H), 2.11 (s, 3H), 2.08-2.00 (m, 1H), 1.99-1.82 (m, 3H), 1.82-1.46 (m, 3H) |
| 418 | 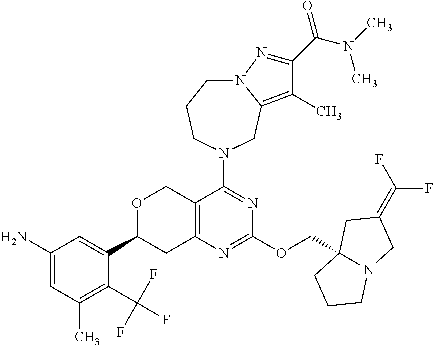 [SMILES: Cc1cc(N)cc([C@@H]2Cc3nc(OC[C@@]45CCCN4CC(=C(F)F)C5)nc(N4CCCn5nc(C(=O)N(C)C)c(C)c5C4)c3CO2)c1C(F)(F)F] | <10 | 737.4 | ¹H NMR (400 MHz, DMSO-d₆) δ: 6.92 (d, J = 2.0 Hz, 1H), 6.74 (d, J = 2.0 Hz, 1H), 6.31 (s, 2H), 5.13-5.04 (m, 2H), 4.86- 4.65 (m, 3H), 4.50- 4.36 (m, 2H), 3.86 (br d, J = 1.6 Hz, 2H), 3.95- 3.79 (m, 1H), 3.34-3.27 (m, 2H), 3.11 (s, 3H), 2.98 (s, 3H), 2.93-2.85 (m, 2H), 2.73-2.63 (m, 2H), 2.58 (s, 3H), 2.16 (s, 3H), 2.13-2.08 (m, 3H), 2.02-1.92 (m, 3H) |
| 419 | 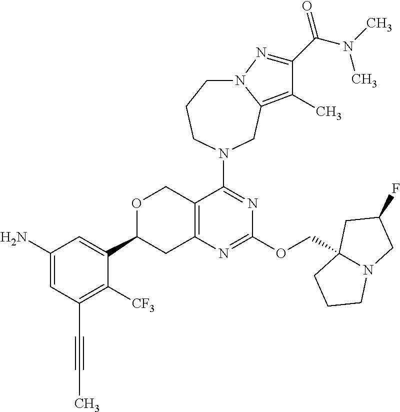 [SMILES: CC#Cc1cc(N)cc([C@@H]2Cc3nc(OC[C@@]45CCCN4C[C@H](F)C5)nc(N4CCCn5nc(C(=O)N(C)C)c(C)c5C4)c3CO2)c1C(F)(F)F] | <10 | 711.4 | ¹H NMR (400 MHz, DMSO-d₆) δ: 6.89-6.83 (m, 1H), 6.69-6.63 (m, 1H), 5.93 (s, 2H), 5.36- 5.16 (m, 1H), 5.04-4.92 (m, 2H), 4.79-4.61 (m, 3H), 4.46-4.30 (m, 2H), 3.94-3.73 (m, 4H), 3.07 (s, 5H), 3.01-2.91 (m, 4H), 2.81 (br d, J = 15.2 Hz, 2H), 2.64 (br dd, J = 11.2, 17.6 Hz, 1H), 2.12 (s, 3H), 2.04 (s, 5H), 2.00-1.89 (m, 3H), 1.83 (br d, J = 7.2 Hz, 1H), 1.78-1.66 (m, 2H) |
| 420 | 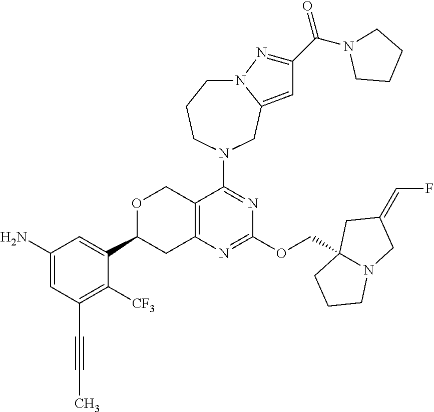 [SMILES: CC#Cc1cc(N)cc([C@@H]2Cc3nc(OC[C@@]45CCCN4C/C(=C\F)C5)nc(N4CCCn5nc(C(=O)N6CCCC6)cc5C4)c3CO2)c1C(F)(F)F] | <10 | 735.4 | ¹H NMR (400 MHz, DMSO-d₆) δ: 7.01-6.75 (m, 2H), 6.71-6.63 (m, 2H), 5.93 (s, 2H), 5.05- 4.91 (m, 2H), 4.79-4.64 (m, 3H), 4.51-4.39 (m, 2H), 4.30-3.94 (m, 3H), 3.93-3.72 (m, 5H), 3.46- 3.39 (m, 3H), 3.06- 2.91 (m, 1H), 2.85-2.77 (m, 1H), 2.60 (br s, 3H), 2.03 (s, 5H), 1.98-1.73 (m, 8H) |
| 421 | 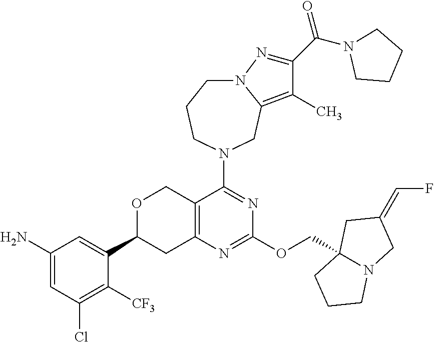 [SMILES: Cc1c(C(=O)N2CCCC2)nn2c1CN(c1nc(OC[C@@]34CCCN3C/C(=C\F)C4)nc3c1CO[C@H](c1cc(N)cc(Cl)c1C(F)(F)F)C3)CCC2] | <10 | 745.3 | ¹H NMR (400 MHz, DMSO-d₆) δ: 7.03-6.68 (m, 3H), 6.21 (s, 2H), 5.03 (m, J = 13.6 Hz, 2H), 4.83-4.62 (m, 3H), 4.47-4.32 (m, 2H), 4.21- 3.97 (m, 2H), 3.82 (m, 3H), 3.67 (m, J = 6.0 Hz, 2H), 3.43 (m, J = 6.4 Hz, 3H), 3.29-3.21 (m, 1H), 3.03-2.78 (m, 2H), 2.69- 2.59 (m, 2H), 2.21 (s, 3H), 2.08 (m, 11H) |
| 422 | 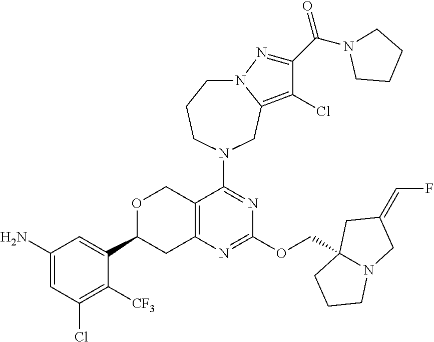 [SMILES: Nc1cc(Cl)c(C(F)(F)F)c([C@@H]2Cc3nc(OC[C@@]45CCCN4C/C(=C\F)C5)nc(N4CCCn5nc(C(=O)N6CCCC6)c(Cl)c5C4)c3CO2)c1] | <10 | 765.3 | ¹H NMR (400 MHz, DMSO-d₆) δ: 6.88-6.61 (m, 3H), 6.24 (s, 2H), 5.13-4.98 (m, 2H), 4.88 (d, J = 16.4 Hz, 1H), 4.76-4.61 (m, 2H), 4.50- 4.37 (m, 2H), 3.94- 3.80 (m, 3H), 3.76-3.68 (m, 1H), 3.64 (d, J = 14.8 Hz, 1H), 3.59-3.50 (m, 2H), 3.45-3.40 (m, 2H), 3.29-3.24 (m, 1H), 3.00- 2.94 (m, 1H), 2.91- 2.81 (m, 1H), 2.68-2.60 (m, 1H), 2.45 (s, 1H), 2.30-2.15 (m, 2H), 2.09- 2.03 (m, 1H), 2.01- 1.88 (m, 1H), 1.87-1.78 (m, 6H), 1.77-1.71 (m, 1H), 1.68-1.62 (m, 1H) |
| 423 | 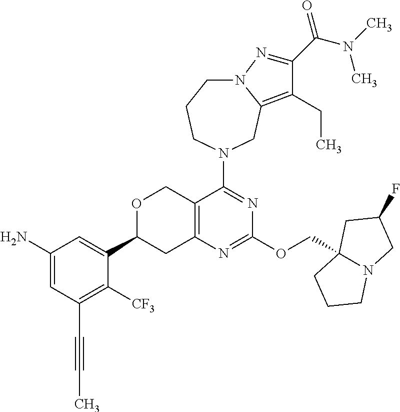 [SMILES: CC#Cc1cc(N)cc([C@@H]2Cc3nc(OC[C@@]45CCCN4C[C@H](F)C5)nc(N4CCCn5nc(C(=O)N(C)C)c(CC)c5C4)c3CO2)c1C(F)(F)F] | <10 | 725.4 | ¹H NMR (400 MHz, DMSO-d₆) δ: 6.88 (s, 1H), 6.69 (d, J = 2.0 Hz, 1H), 6.19 (s, 2H), 5.46- 5.11 (m, 1H), 5.06-4.96 (m, 2H), 4.93-4.82 (m, 1H), 4.76-4.68 (m, 1H), 4.65-4.56 (m, 1H), 4.44- 4.28 (m, 2H), 3.98- 3.78 (m, 3H), 3.77-3.67 (m, 1H), 3.17-2.98 (m, 6H), 2.94 (s, 3H), 2.88- 2.76 (m, 2H), 2.71-2.52 (m, 4H), 2.39-2.19 (m, 1H), 2.15-1.89 (m, 6H), 1.89-1.72 (m, 3H), 1.04- 0.98 (m, 3H) |
| 424 | 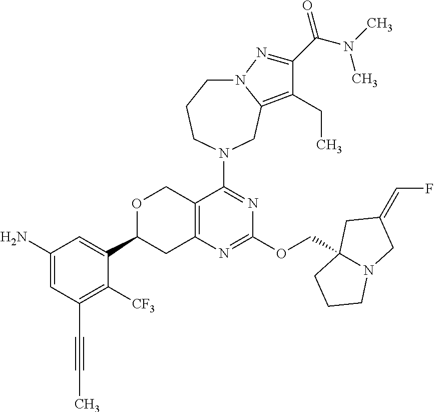 [SMILES: CC#Cc1cc(N)cc([C@@H]2Cc3nc(OC[C@@]45CCCN4C/C(=C\F)C5)nc(N4CCCn5nc(C(=O)N(C)C)c(CC)c5C4)c3CO2)c1C(F)(F)F] | <10 | 737.3 | ¹H NMR (400 MHz, DMSO-d₆) δ: 6.89-6.63 (m, 3H), 6.20 (s, 2H), 5.11-4.88 (m, 3H), 4.73- 4.53 (m, 2H), 4.45- 4.33 (m, 2H), 3.92-3.80 (m, 3H), 3.76-3.62 (m, 2H), 3.26 (s, 1H), 3.03 (s, 3H), 2.99-2.92 (m, 4H), 2.88-2.81 (m, 1H), 2.70-2.54 (m, 4H), 2.35- 2.24 (m, 2H), 2.13- 1.95 (m, 3H), 1.95-1.50 (m, 6H), 1.01 (t, J = 7.6 Hz, 3H) |
| 425 | 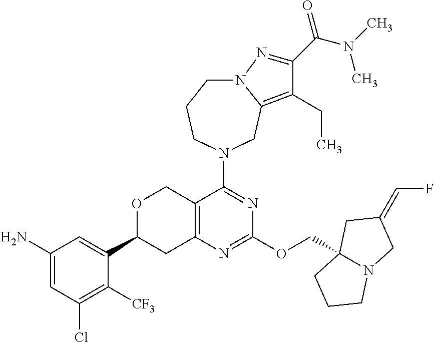 [SMILES: CCc1c(C(=O)N(C)C)nn2c1CN(c1nc(OC[C@@]34CCCN3C/C(=C\F)C4)nc3c1CO[C@H](c1cc(N)cc(Cl)c1C(F)(F)F)C3)CCC2] | <10 | 733.3 | 1H NMR (400 MHz, DMSO-d₆) δ: 6.93-6.63 (m, 3H), 6.20 (s, 2H), 5.10-4.84 (m, 3H), 4.75- 4.56 (m, 2H), 4.46- 4.31 (m, 2H), 3.98-3.78 (m, 3H), 3.77-3.61 (m, 2H), 3.04-2.91 (m, 6H), 2.87-2.79 (m, 1H), 2.69- 2.53 (m, 6H), 2.32- 2.25 (m, 1H), 2.12-1.98 (m, 2H), 1.96-1.53 (m, 5H), 1.01 (t, J = 7.6 Hz, 3H) |
| 426 | 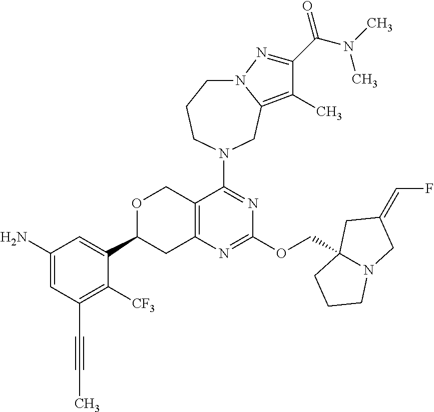 [SMILES: CC#Cc1cc(N)cc([C@@H]2Cc3nc(OC[C@@]45CCCN4C/C(=C\F)C5)nc(N4CCCn5nc(C(=O)N(C)C)c(C)c5C4)c3CO2)c1C(F)(F)F] | <10 | 723.4 | ¹H NMR (400 MHz, DMSO-d₆) δ: 6.89-6.59 (m, 3H), 5.92 (s, 2H), 5.06-4.91 (m, 2H), 4.84- 4.75 (m, 1H), 4.73- 4.57 (m, 2H), 4.45-4.27 (m, 2H), 3.85 (s, 2H), 3.82-3.72 (m, 2H), 3.69- 3.62 (m, 1H), 3.38- 3.33 (m, 1H), 3.30-3.23 (m, 1H), 3.06 (s, 3H), 3.01-2.90 (m, 4H), 2.86- 2.77 (m, 1H), 2.70- 2.59 (m, 1H), 2.57-2.53 (m, 1H), 2.46 (s, 1H), 2.31-2.23 (m, 1H), 2.11 (s, 3H), 2.04 (s, 3H), 2.00-1.93 (m, 1H), 1.91- 1.81 (m, 2H), 1.77- 1.62 (m, 2H) |
| 427 | 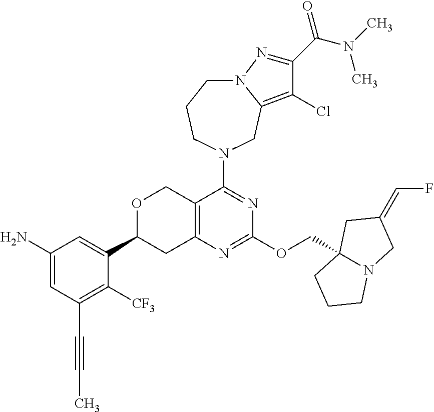 [SMILES: CC#Cc1cc(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F] | <10 | 743.3 | ¹H NMR (400 MHz, DMSO-d₆) δ: 6.89-6.58 (m, 3H), 5.92 (s, 2H), 5.13-4.81 (m, 3H), 4.77- 4.61 (m, 2H), 4.50- 4.31 (m, 2H), 3.99-3.78 (m, 3H), 3.77-3.57 (m, 2H), 3.25 (s, 1H), 2.97 (d, J = 8.4 Hz, 7H), 2.88- 2.76 (m, 1H), 2.65 (dd, J = 10.4, 17.6 Hz, 1H), 2.57-2.52 (m, 1H), 2.45 (s, 1H), 2.30-2.14 (m, 2H), 2.09-1.98 (m, 4H), 1.91-1.79 (m, 2H), 1.77- 1.60 (m, 2H) |
| 428 | 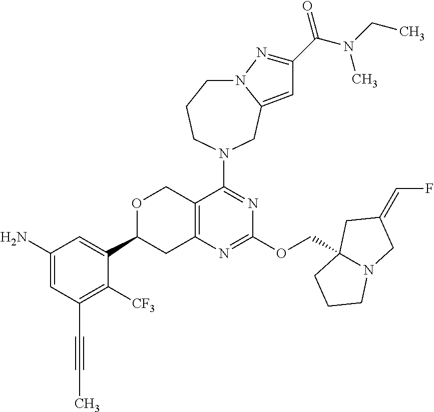 [SMILES: CC#Cc1cc(N)cc([C@@H]2Cc3nc(OC[C@@]45CCCN4C/C(=C\F)C5)nc(N4CCCn5nc(C(=O)N(C)CC)cc5C4)c3CO2)c1C(F)(F)F] | <10 | 723.3 | ¹H NMR (400 MHz, DMSO-d₆) δ: 6.87-6.53 (m, 4H), 5.92 (s, 2H), 5.05-4.93 (m, 2H), 4.76- 4.60 (m, 3H), 4.52- 4.38 (m, 2H), 3.94-3.76 (m, 4H), 3.71-3.61 (m, 2H), 3.45-3.35 (m, 1H), 3.30-3.17 (m, 3H), 3.03- 2.95 (m, 1H), 2.90 (s, 1H), 2.86-2.76 (m, 1H), 2.68-2.54 (m, 2H), 2.49- 2.40 (m, 2H), 2.35- 2.09 (m, 3H), 2.04 (s, 3H), 2.00-1.93 (m, 1H), 1.92-1.59 (m, 5H) |
| 429 | 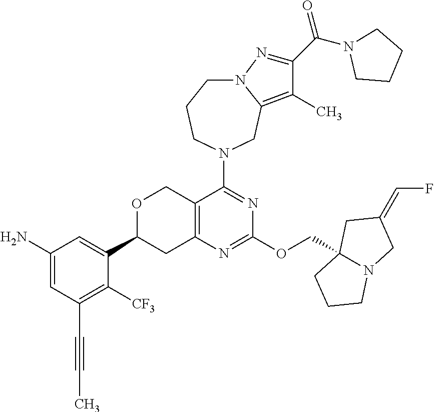 [SMILES: CC#Cc1cc(N)cc([C@@H]2Cc3nc(OC[C@@]45CCCN4C/C(=C\F)C5)nc(N4CCCn5nc(C(=O)N6CCCC6)c(C)c5C4)c3CO2)c1C(F)(F)F] | <10 | 749.4 | ¹H NMR (400 MHz, DMSO-d₆) δ: 6.67-6.64 (m, 1H), 6.86-6.62 (m, 2H), 5.93 (s, 2H), 5.07- 4.87 (m, 2H), 4.84-4.58 (m, 3H), 4.46-4.31 (m, 2H), 3.91-3.72 (m, 4H), 3.71-3.62 (m, 3H), 3.45- 3.38 (m, 2H), 3.30- 3.24 (m, 1H), 3.01-2.91 (m, 1H), 2.87-2.75 (m, 1H), 2.68-2.59 (m, 1H), 2.46 (s, 1H), 2.30-2.24 (m, 1H), 2.20 (s, 3H), 2.10 (s, 1H), 2.04 (s, 3H), 1.99-1.93 (m, 1H), 1.92-1.84 (m, 2H), 1.83- 1.74 (m, 5H), 1.74- 1.61 (m, 2H) |
| 430 | 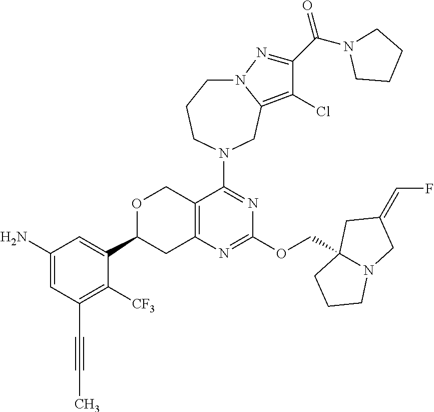 [SMILES: CC#Cc1cc(N)cc([C@@H]2Cc3nc(OC[C@@]45CCCN4C/C(=C\F)C5)nc(N4CCCn5nc(C(=O)N6CCCC6)c(Cl)c5C4)c3CO2)c1C(F)(F)F] | <10 | 769.3 | ¹H NMR (400 MHz, DMSO-d₆) δ: 6.86-6.59 (m, 3H), 6.02-5.81 (m, 2H), 5.11-4.85 (m, 3H), 4.75-4.62 (m, 2H), 4.49- 4.35 (m, 2H), 3.92- 3.80 (m, 3H), 3.76-3.61 (m, 2H), 3.59-3.47 (m, 2H), 3.45-3.38 (m, 2H), 3.29-3.24 (m, 2H), 2.99- 2.93 (m, 1H), 2.87- 2.77 (m, 1H), 2.69-2.61 (m, 1H), 2.42-2.12 (m, 3H), 2.11-2.01 (m, 4H), 1.88-1.77 (m, 6H), 1.75- 1.62 (m, 2H) |
| 431 | 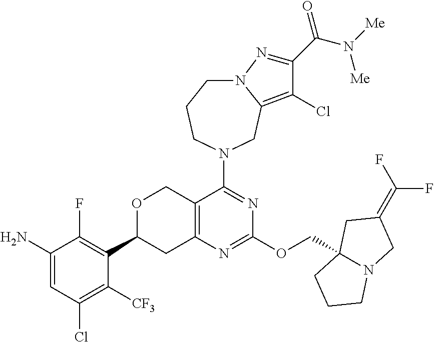 [SMILES: CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2] | <10 | 775.3 | ¹H NMR (400 MHz, CDCl₃) δ 6.84 (d, J = 7.6 Hz, 1H), 5.20 (br dd, J = 3.6, 11.2 Hz, 1H), 4.98 (br d, J = 13.6 Hz, 1H), 4.84 (d, J = 16.4 Hz, 1H), 4.69 (d, J = 13.6 Hz, 1H), 4.56-4.47 (m, 1H), 4.43 (d, J = 16.4 Hz, 1H), 4.39- 4.31 (m, 1H), 4.19 (s, 2H), 4.10-4.04 (m, 1H), 4.03-3.97 (m, 1H), 3.97- 3.89 (m, 1H), 3.76- 3.61 (m, 2H), 3.41-3.28 (m, 2H), 3.17-3.12 (m, 1H), 3.10 (s, 6H), 2.98 (br dd, J = 3.6, 18.0 Hz, 1H), 2.76 (br d, J = 15.2 Hz, 1H), 2.67-2.58 (m, 1H), 2.41-2.30 (m, 2H), 2.19-2.11 (m, 2H), 1.96- 1.88 (m, 2H), 1.87 (br s, 1H) |
| 432 | 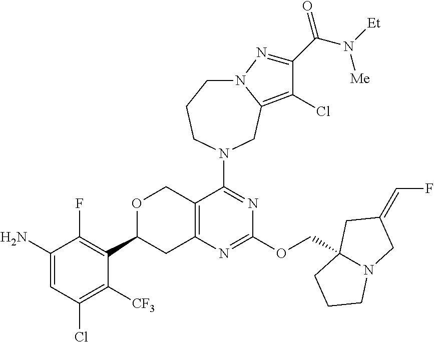 [SMILES: CCN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2] | <10 | 771.3 | ¹H NMR (400 MHz, CDCl₃) δ 6.84 (d, J = 7.6 Hz, 1H), 6.62-6.35 (m, 1H), 5.21 (br dd, J = 11.2, 3.2 Hz, 1H), 4.99 (br d, J = 13.6 Hz, 1H), 4.92-4.78 (m, 1H), 4.69 (br d, J = 13.6 Hz, 1H), 4.57-4.46 (m, 1H), 4.44- 4.31 (m, 2H), 4.17 (s, 2H), 4.08-3.92 (m, 3H), 3.89-3.79 (m, 1H), 3.70- 3.53 (m, 2H), 3.48- 3.27 (m, 3H), 3.22-3.11 (m, 1H), 3.06 (d, J = 5.2 Hz, 3H), 3.02-2.93 (m, 1H), 2.75-2.57 (m, 2H), 2.38-2.27 (m, 2H), 2.19- 2.10 (m, 2H), 1.94- 1.76 (m, 3H), 1.25-1.17 (m, 3H) |
| 433 | 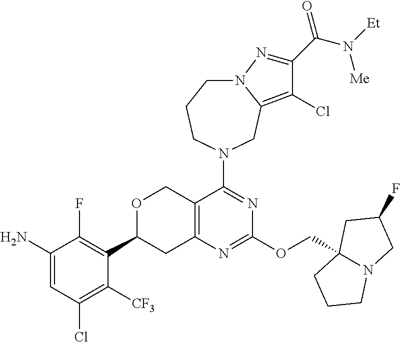 [SMILES: CCN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2] | <10 | 759.3 | ¹H NMR (400 MHz, CDCl₃) δ 6.8 (d, J = 7.6 Hz, 1 H) 5.2-5.3 (m, 2 H) 5.0 (br d, J = 13.6 Hz, 1 H) 4.7-4.9 (m, 1 H) 4.7 (br d, J = 13.2 Hz, 1 H) 4.4-4.5 (m, 2 H) 4.3- 4.4 (m, 1 H) 4.2 (s, 2 H) 3.9-4.1 (m, 3 H) 3.5- 3.7 (m, 2 H) 3.4 (q, J = 7.2 Hz, 1 H) 3.2-3.4 (m, 3 H) 3.1 (br s, 1 H) 3.1 (d, J = 2.4 Hz, 3 H) 3.0 (br dd, J = 14.4, 4.8 Hz, 2 H) 2.2-2.4 (m, 2 H) 2.1- 2.2 (m, 3 H) 1.8-2.0 (m, 3 H) 1.2 (dt, J = 16.8, 7.2 Hz, 3 H) |
| 434 | 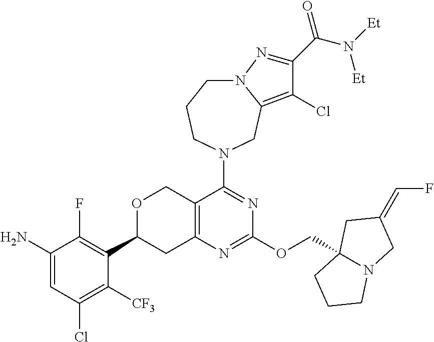 [SMILES: CCN(CC)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2] | <10 | 785.3 | ¹H NMR (400 MHz, CDCl₃) δ 6.89-6.78 (m, 1H), 6.69-6.32 (m, 1H), 5.21 (br dd, J = 10.4, 3.6 Hz, 1H), 4.99 (br d, J = 13.6 Hz, 1H), 4.91-4.80 (m, 1H), 4.69 (d, J = 13.6 Hz, 1H), 4.50 (br dd, J = 14.0, 5.6 Hz, 1H), 4.44- 4.31 (m, 2H), 4.17 (s, 2H), 4.10-3.98 (m, 2H), 3.98-3.90 (m, 1H), 3.88- 3.79 (m, 1H), 3.71- 3.60 (m, 1H), 3.59-3.49 (m, 2H), 3.46-3.37 (m, 3H), 3.37-3.27 (m, 1H), 3.22-3.11 (m, 1H), 3.03- 2.92 (m, 1H), 2.74- 2.67 (m, 1H), 2.66-2.56 (m, 1H), 2.38-2.26 (m, 2H), 2.19-2.09 (m, 2H), 1.95-1.85 (m, 2H), 1.81- 1.74 (m, 1H), 1.24 (br t, J = 7.2 Hz, 3H), 1.17 (br t, J = 7.2 Hz, 3H) |
| 435 | 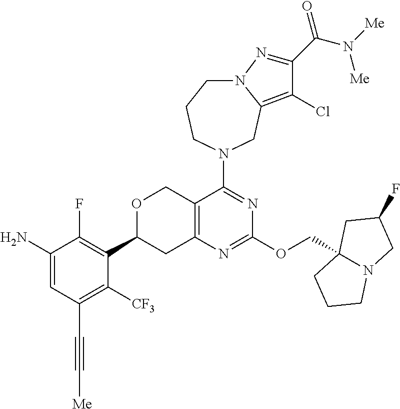 [SMILES: CC#Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F] | <10 | 749.4 | ¹H NMR (400 MHz, CDCl₃) δ 6.82 (d, J = 8.4 Hz, 1H), 5.44-5.16 (m, 2H), 4.99 (d, J = 13.6 Hz, 1H), 4.81-4.70 (m, 2H), 4.54-4.33 (m, 2H), 4.40- 4.26 (m, 1H), 4.22- 4.10 (m, 1H), 4.10-3.99 (m, 2H), 3.98-3.89 (m, 1H), 3.68-3.61 (m, 1H), 3.44-3.30 (m, 2H), 3.27- 3.14 (m, 2H), 3.11 (s, 6H), 3.05-2.91 (m, 1H), 2.39-2.28 (m, 4H), 2.28- 2.12 (m, 2H), 2.05 (s, 3H), 2.02-1.85 (m, 2H). |
| 436 | 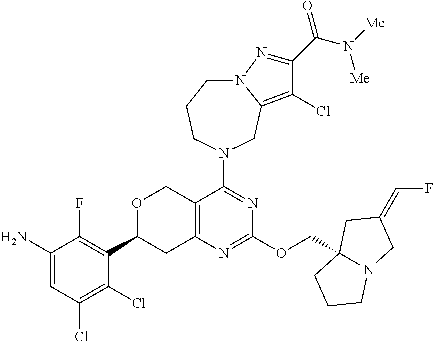 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1Cl)C3)CCC2] | <10 | 725.2 | ¹H NMR (400 MHz, CDCl₃) δ ppm 6.90 (d, J = 8.4 Hz, 1 H) 6.33- 6.69 (m, 1 H) 5.37 (dd, J = 11.6, 4.4 Hz, 1 H) 5.03 (br d, J = 13.6 Hz, 1 H) 4.61-4.91 (m, 2 H) 4.31- 4.57 (m, 3 H) 3.72- 4.30 (m, 6 H) 3.60-3.71 (m, 1 H) 3.28-3.56 (m, 2 H) 3.10 (d, J = 3.6 Hz, 6 H) 2.88-3.00 (m, 1 H) 2.50-2.85 (m, 2 H) 2.27- 2.45 (m, 2 H) 2.07- 2.26 (m, 2 H) 1.74-2.04 (m, 3 H) 1.72 (br s, 1 H) |
| 437 | 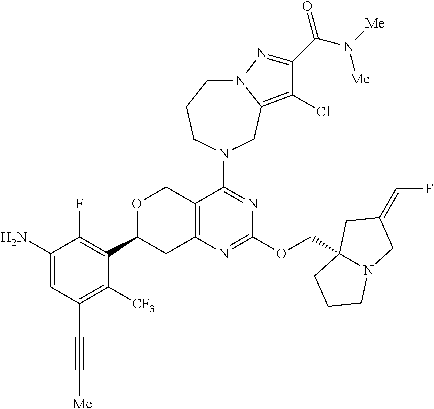 [SMILES: CC#Cc1cc(N)c(F)c([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F] | <10 | 761.3 | ¹H NMR (400 MHz, CDCl₃) δ 6.84 (d, J = 8.4 Hz, 1H), 6.71-6.36 (m, 1H), 5.16 (br dd, J = 11.2, 4.0 Hz, 1H), 5.01 (br d, J = 14.0 Hz, 1H), 4.85-4.67 (m, 2H), 4.51 (br dd, J = 12.4, 6.4 Hz, 2H), 4.35 (br dd, J = 13.6, 10.4 Hz, 2H), 4.18 (br s, 1H), 4.04 (s, 4H), 3.67-3.60 (m, 1H), 3.37- 3.30 (m, 1H), 3.14 (br d, J = 6.0 Hz, 1H), 3.10 (s, 6H), 2.95 (br dd, J = 18.0, 3.2 Hz, 1H), 2.86- 2.58 (m, 2H), 2.43-2.30 (m, 2H), 2.29-2.25 (m, 1H), 2.30-2.06 (m, 2H), 2.05 (s, 3H), 2.02-1.71 (m, 3H) |
| 438 | 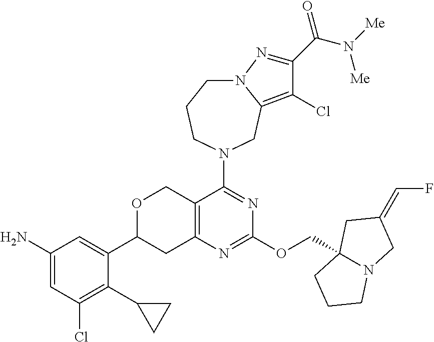 [SMILES: CN(C)C(=O)c1nn2c(c1Cl)CN(c1nc(OC[C@@]34CCCN3C/C(=C\F)C4)nc3c1COC(c1cc(N)cc(Cl)c1C1CC1)C3)CCC2] | <10 | 711.4 | ¹H NMR (400 MHz, CDCl₃) δ 6.71 (d, J = 2.4 Hz, 1H), 6.68 (d, J = 2.4 Hz, 1H), 6.63-6.38 (m, 1H), 5.47 (dd, J = 11.2, 3.6 Hz, 1H), 5.09 (br d, J = 13.6 Hz, 1H), 4.80 (br d, J = 16.4 Hz, 1H), 4.70 (d, J = 13.6 Hz, 1H), 4.57- 4.35 (m, 3H), 4.05 (br d, J = 13.6 Hz, 2H), 3.95- 3.80 (m, 1H), 3.73- 3.59 (m, 3H), 3.51-3.39 (m, 1H), 3.31-3.14 (m, 1H), 3.10 (s, 6H), 3.06- 3.00 (m, 1H), 2.94-2.86 (m, 1H), 2.78-2.61 (m, 2H), 2.44-2.30 (m, 2H), 2.21-2.09 (m, 2H), 1.98- 1.86 (m, 2H), 1.84- 1.76 (m, 1H), 1.66-1.61 (m, 2H), 1.11-0.99 (m, 2H), 0.66 (br d, J = 4.4 Hz, 2H) |
| 439 | 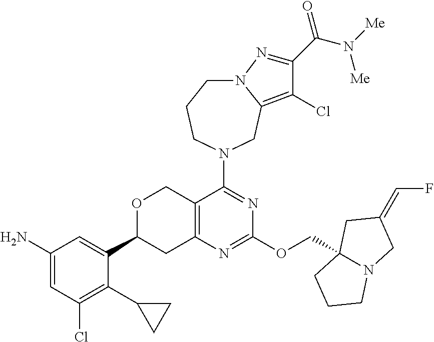 [SMILES: CN(C)C(=O)c1nn2c(c1Cl)CN(c1nc(OC[C@@]34CCCN3C/C(=C\F)C4)nc3c1CO[C@H](c1cc(N)cc(Cl)c1C1CC1)C3)CCC2] | <10 | 711.4 | ¹H NMR (400 MHz, CDCl₃) δ 6.71 (d, J = 2.4 Hz, 1H), 6.68 (d, J = 2.4 Hz, 1H), 6.63-6.34 (m, 1H), 5.47 (dd, J = 11.2, 3.6 Hz, 1H), 5.09 (br d, J = 13.2 Hz, 1H), 4.84- 4.75 (m, 1H), 4.71 (br d, J = 13.6 Hz, 1H), 4.55- 4.35 (m, 3H), 4.14-4.02 (m, 2H), 3.93-3.75 (m, 1H), 3.71-3.57 (m, 3H), 3.53-3.35 (m, 1H), 3.23- 3.12 (m, 1H), 3.11- 3.05 (m, 6H), 3.02 (br d, J = 3.2 Hz, 1H), 2.95- 2.86 (m, 1H), 2.81-2.60 (m, 2H), 2.48-2.32 (m, 2H), 2.26-2.11 (m, 2H), 2.05-1.89 (m, 2H), 1.86- 1.76 (m, 1H), 1.61 (br s, 2H), 1.10-1.01 (m, 2H), 0.70-0.60 (m, 2H) |
| 440 | 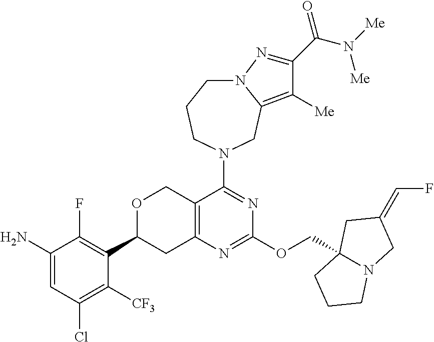 [SMILES: Cc1c(C(=O)N(C)C)nn2c1CN(c1nc(OC[C@@]34CCCN3C/C(=C\F)C4)nc3c1CO[C@H](c1c(F)c(N)cc(Cl)c1C(F)(F)F)C3)CCC2] | <10 | 737.4 | ¹H NMR (400 MHz, CDCl₃) δ 6.84 (d, J = 7.6 Hz, 1H), 6.75-6.30 (m, 1H), 5.18 (br dd, J = 11.2, 3.2 Hz, 1H), 5.03- 4.90 (m, 1H), 4.86 (br d, J = 13.6 Hz, 1H), 4.71 (br d, J = 13.6 Hz, 1H), 4.54-4.44 (m, 1H), 4.56- 4.25 (m, 3H), 4.18 (s, 2H), 4.12-3.99 (m, 1H), 3.97-3.74 (m, 2H), 3.71- 3.44 (m, 2H), 3.36- 3.28 (m, 1H), 3.18-3.05 (m, 6H), 2.96 (br dd, J = 17.6, 4.2 Hz, 1H), 2.84- 2.24 (m, 4H), 2.19 (s, 3H), 2.16-1.84 (m, 4H) |
| 441 | 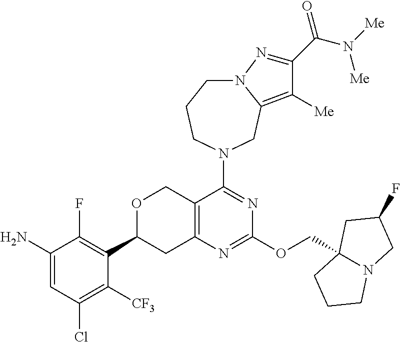 [SMILES: Cc1c(C(=O)N(C)C)nn2c1CN(c1nc(OC[C@@]34CCCN3C[C@H](F)C4)nc3c1CO[C@H](c1c(F)c(N)cc(Cl)c1C(F)(F)F)C3)CCC2] | <10 | 725.3 | ¹H NMR (400 MHz, CDCl₃) δ 6.83 (d, J = 8.0 Hz, 1H), 5.42-5.09 (m, 2H), 4.94 (br d, J = 16.0 Hz, 1H), 4.88-4.79 (m, 1H), 4.70 (d, J = 13.2 Hz, 1H), 4.53-4.42 (m, 1H), 4.39-4.28 (m, 2H), 4.19 (s, 2H), 4.13-4.05 (m, 1H), 4.04-3.90 (m, 1H), 3.86-3.76 (m, 1H), 3.71- 3.57 (m, 1H), 3.55- 3.17 (m, 4H), 3.17 (br s, 3H), 3.08 (br s, 3H), 3.01- 2.92 (m, 2H), 2.36- 2.19 (m, 2H), 2.19-2.01 (m, 6H), 1.98-1.80 (m, 3H) |
| 442 | 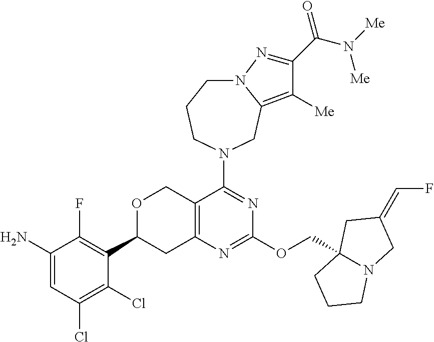 [SMILES: Cc1c(C(=O)N(C)C)nn2c1CN(c1nc(OC[C@@]34CCCN3C/C(=C\F)C4)nc3c1CO[C@H](c1c(F)c(N)cc(Cl)c1Cl)C3)CCC2] | <10 | 703.3 | ¹H NMR (400 MHz, CDCl₃) δ ppm 6.90 (d, J = 8.4 Hz, 1 H) 6.31- 6.71 (m, 1 H) 5.35 (dd, J = 11.6, 4.4 Hz, 1 H) 4.84- 5.02 (m, 2 H) 4.73 (br d, J = 13.6 Hz, 1 H) 4.31- 4.53 (m, 3 H) 4.00- 4.29 (m, 2 H) 3.75-4.00 (m, 4 H) 3.63-3.74 (m, 1 H) 3.41-3.59 (m, 1 H) 3.34 (br dd, J = 17.6, 11.6 Hz, 1 H) 3.06-3.20 (m, 6 H) 2.89-2.98 (m, 1 H) 2.61-2.89 (m, 2 H) 2.29-2.43 (m, 1 H) 2.09- 2.27 (m, 6 H) 1.70- 2.08 (m, 4 H) |
| 443 | 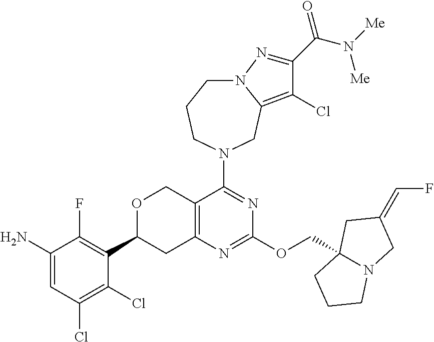 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1Cl)C3)CCC2] | <10 | 707.2 | ¹H NMR (400 MHz, CDCl₃) δ 6.8 (d, J = 2.8 Hz, 1 H) 6.7-6.8 (m, 1 H) 6.3-6.7 (m, 1 H) 5.0- 5.1 (m, 2 H) 4.7-4.8 (m, 2 H) 4.5-4.6 (m, 2 H) 4.3-4.4 (m, 1 H) 3.9- 4.2 (m, 3 H) 3.8 (br s, 2 H) 3.7-3.7 (m, 1 H) 3.4- 3.6 (m, 1 H) 3.3 (br dd, J = 17.6, 3.2 Hz, 2 H) 3.1 (d, J = 3.2 Hz, 6 H) 2.6- 2.8 (m, 3 H) 2.3-2.4 (m, 2 H) 2.1-2.2 (m, 2 H) 1.7-2.1 (m, 4 H) |
| 444 | 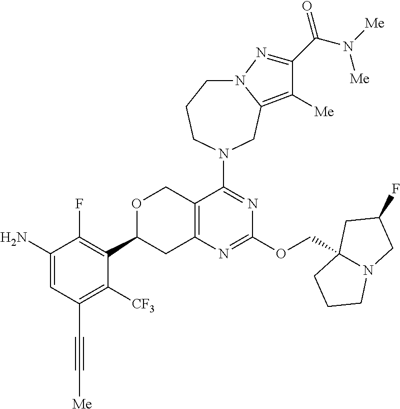 [SMILES: CC#Cc1cc(N)c(F)c([C@@H]2Cc3nc(OC[C@@]45CCCN4C[C@H](F)C5)nc(N4CCCn5nc(C(=O)N(C)C)c(C)c5C4)c3CO2)c1C(F)(F)F] | <10 | 729.3 | ¹H NMR (400 MHz, CDCl₃) δ 6.87 (d, J = 8.4 Hz, 1H), 5.39-5.20 (m, 1H), 5.18-5.10 (m, 1H), 4.99-4.81 (m, 2H), 4.71 (d, J = 13.2 Hz, 1H), 4.51- 4.44 (m, 1H), 4.39- 4.29 (m, 2H), 4.09 (br d, J = 13.6 Hz, 2H), 4.05 (s, 2H), 3.96-3.47 (m, 3H), 3.31 (br dd, J = 18.0, 11.2 Hz, 3H), 3.18-3.07 (m, 6H), 3.04-2.90 (m, 2H), 2.29 (br d, J = 1.2 Hz, 2H), 2.19 (s, 3H), 2.13 (br d, J = 3.2 Hz, 2H), 2.04 (s, 3H), 1.97 (br d, J = 1.6 Hz, 4H) |
| 445 | 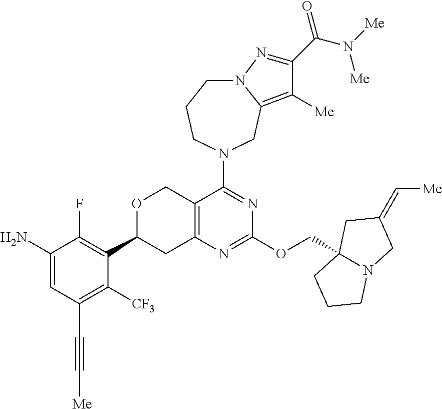 [SMILES: CC#Cc1cc(N)c(F)c([C@@H]2Cc3nc(OC[C@@]45CCCN4C/C(=C\C)C5)nc(N4CCCn5nc(C(=O)N(C)C)c(C)c5C4)c3CO2)c1C(F)(F)F] | <10 | 741.4 | ¹H NMR (400 MHz, CDCl₃) δ 6.87 (d, J = 8.4 Hz, 1H), 6.66-6.38 (m, 1H), 5.17-5.11 (m, 1H), 5.03-4.84 (m, 2H), 4.70 (d, J = 13.6 Hz, 1H), 4.54- 4.45 (m, 1H), 4.38- 4.29 (m, 2H), 4.11-4.02 (m, 3H), 3.94-3.61 (m, 3H), 3.46 (br s, 1H), 3.32 (br dd, J = 17.6, 11.2 Hz, 2H), 3.18-3.06 (m, 6H), 2.94 (br dd, J = 17.6, 3.2 Hz, 1H), 2.80-2.62 (m, 2H), 2.41-2.22 (m, 2H), 2.18 (s, 3H), 2.17-2.09 (m, 2H), 2.04 (s, 3H), 1.95 (br s, 4H) |
| 446 | 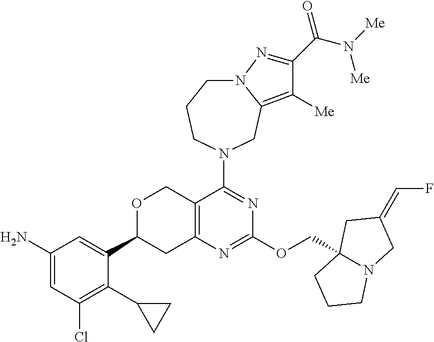 [SMILES: Cc1c(C(=O)N(C)C)nn2c1CN(c1nc(OC[C@@]34CCCN3C/C(=C\F)C4)nc3c1CO[C@H](c1cc(N)cc(Cl)c1C1CC1)C3)CCC2] | <10 | 725.3 | ¹H NMR (400 MHz, CDCl₃) δ 6.71 (d, J = 2.4Hz, 1H), 6.67 (d, J = 2.4 Hz, 1H), 6.53 (s, 1H), 5.44 (dd, J = 4.0, 10.8 Hz, 1H), 4.99-4.86 (m, 2H), 4.73 (d, J = 13.6 Hz, 1H), 4.53-4.46 (m, 1H), 4.43-4.32 (m, 2H), 4.29- 4.00 (m, 2H), 3.99- 3.80 (m, 2H), 3.74-3.62 (m, 3H), 3.56-3.35 (m, 1H), 3.33-3.18 (m, 1H), 3.17-3.05 (m, 5H), 3.04- 2.95 (m, 1H), 2.93- 2.85 (m, 1H), 2.84 (br d, J = 1.2 Hz, 2H), 2.45- 2.33 (m, 1H), 2.20 (s, 3H), 2.19-2.09 (m, 2H), 2.06-1.92 (m, 2H), 1.89- 1.70 (m, 2H), 1.05 (br t, J = 9.2 Hz, 2H), 0.70- 0.60 (m, 2H) |
| 447 | 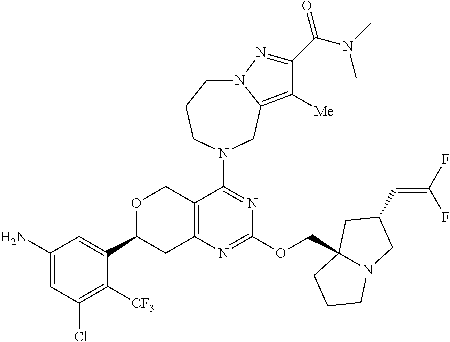 [SMILES: Cc1c(C(=O)N(C)C)nn2c1CN(c1nc(OC[C@]34CCCN3C[C@@H](C=C(F)F)C4)nc3c1CO[C@H](c1cc(N)cc(Cl)c1C(F)(F)F)C3)CCC2] | <10 | 751.2 | ¹H NMR (400 MHz, MeOD) δ 6.94 (d, J = 2.3 Hz, 1H), 6.71 (d, J = 2.2 Hz, 1H), 5.12 (d, J = 8.3 Hz, 1H), 4.99 (t, J = 15.4 Hz, 2H), 4.74 (d, J = 13.7 Hz, 1H), 4.63 (d, J = 16.2 Hz, 1H), 4.50-4.43 (m, 1H), 4.40- 4.26 (m, 2H), 4.04 (s, 2H), 3.96-3.90 (m, 1H), 3.82-3.74 (m, 1H), 3.23- 3.18 (m, 1H), 3.11 (s, 3H), 3.08 (d, J = 8.4 Hz, 4H), 3.00 (d, J = 3.1 Hz, 1H), 2.98-2.89 (m, 2H), 2.79-2.67 (m, 2H), 2.42- 2.36 (m, 1H), 2.27 (dd, J = 12.6, 6.4 Hz, 1H), 2.22-2.15 (m, 4H), 2.12 (s, 1H), 2.04-1.95 (m, 1H), 1.86 (dd, J = 7.7, 4.4 Hz, 1H), 1.81-1.75 (m, 1H), 1.47 (t, J = 12.3 Hz, 1H) |
| 448 | 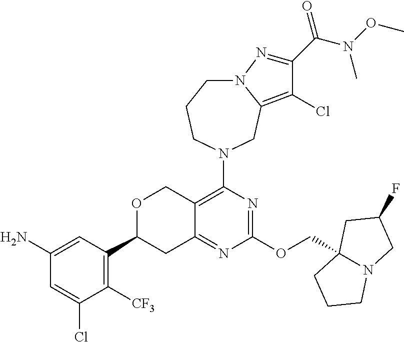 [SMILES: CON(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2] | <10 | 743.2 | ¹H NMR (400 MHz, MeOD) δ 6.96 (d, J = 2.1 Hz, 1H), 6.72 (d, J = 2.2 Hz, 1H), 5.35-5.19 (m, 1H), 5.17-5.12 (m, 1H), 5.06 (d, J = 13.8 Hz, 1H), 4.81 (d, J = 13.6 Hz, 1H), 4.75 (d, J = 7.1 Hz, 1H), 4.48 (dd, J = 14.2, 6.3 Hz, 1H), 4.29 (dd, J = 13.3, 9.9 Hz, 1H), 4.08 (d, J = 10.6 Hz, 1H), 3.96- 3.89 (m, 2H), 3.81- 3.72 (m, 4H), 3.37 (s, 3H), 3.18 (ddd, J = 21.6, 11.1, 8.5 Hz, 3H), 2.98 (dd, J = 23.1, 6.4 Hz, 2H), 2.71 (dd, J = 17.9, 10.8 Hz, 1H), 2.39-2.02 (m, 6H), 1.97-1.82 (m, 3H) |
| 449 | 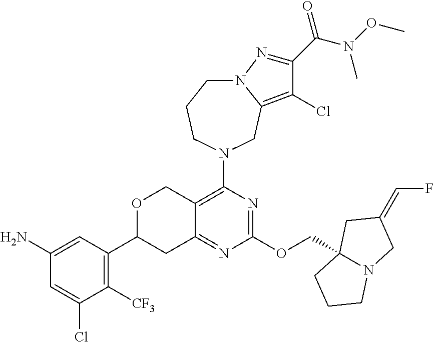 [SMILES: CON(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2] | <10 | 755.2 | ¹H NMR (400 MHz, MeOD) δ 7.00-6.71 (m, 3H), 5.17 (d, J = 8.4 Hz, 1H), 5.07 (d, J = 13.8 Hz, 1H), 4.98 (dd, J = 16.3, 2.3 Hz, 1H), 4.83 (s, 1H), 4.66 (d, J = 16.3 Hz, 1H), 4.50 (m, 2H), 4.41-4.19 (m, 3H), 4.11 (d, J = 14.9 Hz, 1H), 3.95 (m, 1H), 3.84 (m, 1H), 3.79-3.69 (m, 4H), 3.37 (s, 3H), 3.29-3.21 (m, 1H), 3.06- 2.87 (m, 2H), 2.76 (m, 2H), 2.22 (m, 6H) |
| 456 | 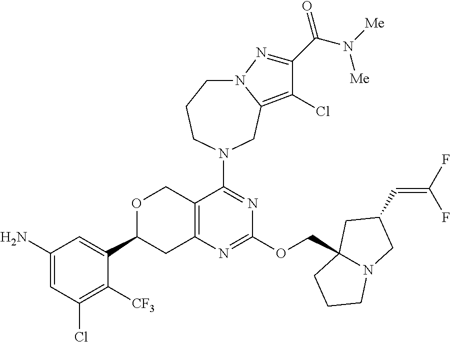 [SMILES: CN(C)C(=O)c1nn2c(c1Cl)CN(c1nc(OC[C@]34CCCN3C[C@@H](C=C(F)F)C4)nc3c1CO[C@H](c1cc(N)cc(Cl)c1C(F)(F)F)C3)CCC2] | <10 | 771.2 | ¹H NMR (400 MHz, MeOD) δ 6.95 (d, J = 2.2 Hz, 1H), 6.72 (d, J = 2.3 Hz, 1H), 5.11 (dd, J = 24.0, 11.0 Hz, 2H), 4.88 (s, 1H), 4.81-4.73 (m, 2H), 4.49 (dd, J = 14.1, 5.8 Hz, 1H), 4.39-4.25 (m, 2H), 4.14 (d, J = 10.7 Hz, 1H), 3.97 (d, J = 10.7 Hz, 2H), 3.77 (dd, J = 12.0, 8.3 Hz, 1H), 3.24- 3.17 (m, 1H), 3.10 (s, 3H), 3.08 (s, 3H), 2.98 (dd, J = 26.0, 8.3 Hz, 2H), 2.80-2.67 (m, 2H), 2.44-2.24 (m, 3H), 2.18 (d, J = 7.3 Hz, 1H), 2.05- 1.71 (m, 5H), 1.48 (t, J = 12.3 Hz, 1H) |
| 457 | 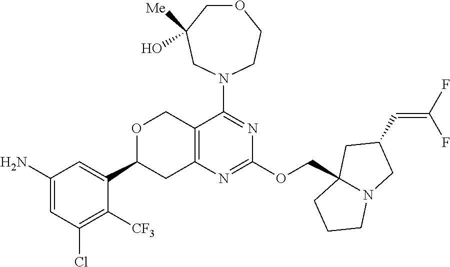 [SMILES: C[C@@]1(O)COCCN(c2nc(OC[C@]34CCCN3C[C@@H](C=C(F)F)C4)nc3c2CO[C@H](c2cc(N)cc(Cl)c2C(F)(F)F)C3)C1] | <10 | 660.1 | ¹H NMR (400 MHz, MeOD) δ 6.98 (d, J = 2.1 Hz, 1H), 6.73 (d, J = 2.2 Hz, 1H), 5.13-5.03 (m, 2H), 4.82 (d, J = 5.0 Hz, 1H), 4.46-4.34 (m, 3H), 4.22-4.11 (m, 2H), 4.00 (d, J = 15.3 Hz, 1H), 3.94- 3.87 (m, 1H), 3.71- 3.61 (m, 2H), 3.45-3.35 (m, 4H), 3.28-3.22 (m, 2H), 2.96 (d, J = 16.9 Hz, 1H), 2.85 (t, J = 11.5 Hz, 1H), 2.73 (dd, J = 17.8, 10.7 Hz, 1H), 2.46 (dd, J = 13.6, 6.4 Hz, 1H), 2.30- 2.20 (m, 1H), 2.19- 2.09 (m, 2H), 2.08-2.01 (m, 1H), 1.83 (t, J = 12.6 Hz, 1H), 1.18 (d, J = 4.3 Hz, 3H) |
| 458 | 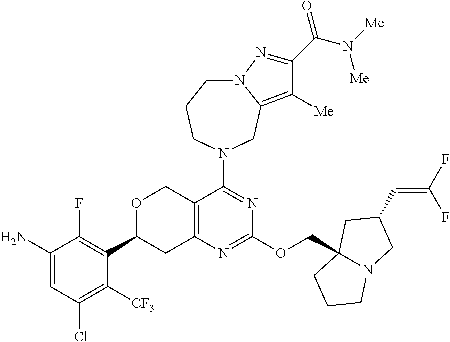 [SMILES: Cc1c(C(=O)N(C)C)nn2c1CN(c1nc(OC[C@]34CCCN3C[C@@H](C=C(F)F)C4)nc3c1CO[C@H](c1c(F)c(N)cc(Cl)c1C(F)(F)F)C3)CCC2] | <10 | 769.2 | ¹H NMR (400 MHz, CD₃OD) δ 6.90 (d, J = 7.9 Hz, 1H), 5.21 (dd, J = 11.4, 4.3 Hz, 1H), 4.97- 4.89 (m, 2H), 4.65 (dd, J = 24.5, 14.9 Hz, 2H), 4.49-4.43 (m, 1H), 4.39- 4.26 (m, 2H), 4.08- 4.02 (m, 2H), 3.93 (dt, J = 8.0, 3.8 Hz, 1H), 3.78- 3.71 (m, 1H), 3.23-3.19 (m, 2H), 3.11 (s, 3H), 3.07 (s, 3H), 2.97-2.85 (m, 2H), 2.79-2.74 (m, 1H), 2.38 (dd, J = 11.0, 9.4 Hz, 1H), 2.33-2.20 (m, 2H), 2.17 (s, 3H), 2.14-1.75 (m, 6H), 1.47 (t, J = 12.3 Hz, 1H). |
| 459 | 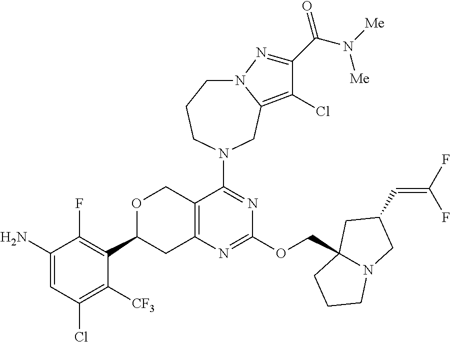 [SMILES: CN(C)C(=O)c1nn2c(c1Cl)CN(c1nc(OC[C@]34CCCN3C[C@@H](C=C(F)F)C4)nc3c1CO[C@H](c1c(F)c(N)cc(Cl)c1C(F)(F)F)C3)CCC2] | <10 | 789.2 | ¹H NMR (400 MHz, MeOD) d 6.91 (d, J = 7.9 Hz, 1H), 5.25 (dd, J = 10.9, 4.2 Hz, 1H), 4.95 (s, 2H), 4.75 (d, J = 13.9 Hz, 1H), 4.62 (d, J = 16.5 Hz, 1H), 4.53-4.38 (m, 3H), 4.22-4.13 (m, 2H), 3.93 (d, J = 13.7 Hz, 1H), 3.78 (dt, J = 14.1, 6.8 Hz, 2H), 3.53 (dd, J = 11.8, 6.3 Hz, 1H), 3.41-3.36 (m, 2H), 3.25 (d, J = 6.8 Hz, 1H), 3.11 (t, J = 7.7 Hz, 6H), 2.99-2.89 (m, 2H), 2.56 (dd, J = 13.4, 6.2 Hz, 1H), 2.43-2.28 (m, 2H), 2.26-2.07 (m, 4H), 1.96-1.87 (m, 1H). |
| 460 | 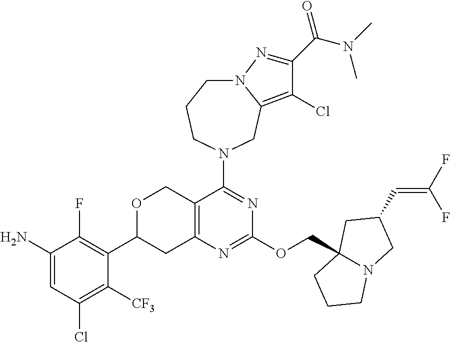 [SMILES: CN(C)C(=O)c1nn2c(c1Cl)CN(c1nc(OC[C@]34CCCN3C[C@@H](C=C(F)F)C4)nc3c1COC(c1c(F)c(N)cc(Cl)c1C(F)(F)F)C3)CCC2] | <10 | 789.2 | ¹H NMR (400 MHz, MeOD) d 6.94 (d, J = 7.9 Hz, 1H), 5.28 (d, J = 11.1 Hz, 1H), 4.99 (t, J = 12.2 Hz, 2H), 4.80 (dd, J = 13.8, 4.4 Hz, 1H), 4.68 (d, J = 16.4 Hz, 1H), 4.61- 4.43 (m, 3H), 4.40 (dd, J = 13.6, 7.1 Hz, 1H), 4.23 (dd, J = 22.1, 11.3 Hz, 2H), 3.96 (s, 1H), 3.82 (dd, J = 11.0, 5.3 Hz, 2H), 3.53 (s, 1H), 3.43 (d, J = 5.8 Hz, 1H), 3.31-3.25 (m, 1H), 3.13 (dd, J = 12.2, 2.3 Hz, 6H), 3.03-2.92 (m, 2H), 2.55 (ddd, J = 30.6, 13.2, 6.8 Hz, 1H), 2.47-2.31 (m, 2H), 2.20 (ddt, J = 19.2, 11.1, 5.5 Hz, 4H), 1.95 (t, J = 12.9 Hz, 1H) |
| 461 | 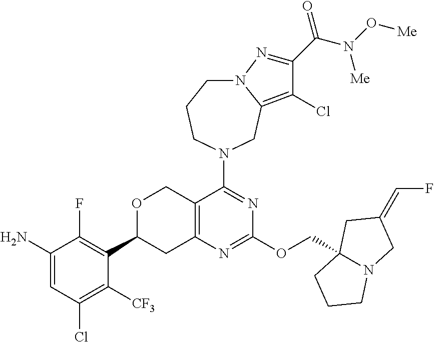 [SMILES: CON(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2] | <10 | 773.1 | ¹H NMR (400 MHz, MeOD) δ 6.92 (dd, J = 44.7, 36.7 Hz, 2H), 5.28 (dd, J = 10.9, 3.9 Hz, 1H), 4.98 (dd, J = 15.0, 5.8 Hz, 3H), 4.71 (dd, J = 62.3, 15.1 Hz, 1H), 4.50 (dd, J = 13.1, 6.3 Hz, 2H), 4.40 (d, J = 14.6 Hz, 1H), 4.33-4.20 (m, 2H), 4.14 (d, J = 14.6 Hz, 1H), 4.02-3.91 (m, 1H), 3.85- 3.74 (m, 5H), 3.40 (s, 3H), 3.30-3.22 (m, 2H), 2.93 (d, J = 17.7 Hz, 2H), 2.74 (d, J = 16.0 Hz, 1H), 2.39 (dd, J = 10.2, 5.0 Hz, 2H), 2.28-2.11 (m, 4H) |
| 462 | 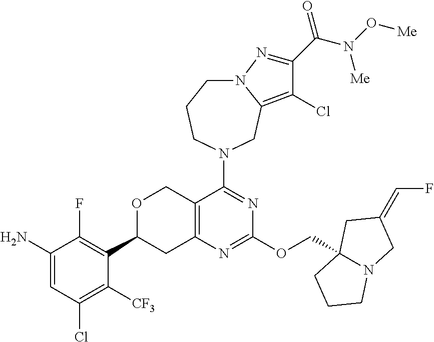 [SMILES: CON(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2] | <10 | 723.2 | ¹H NMR (400 MHz, MeOD) δ 7.25 (d, J = 8.6 Hz, 1H), 6.85 (t, J = 8.5 Hz, 1H), 6.74-6.49 (m, 1H), 5.14 (dd, J = 11.2, 3.9 Hz, 1H), 5.05 (d, J = 13.6 Hz, 1H), 4.77 (dd, J = 33.0, 16.6 Hz, 3H), 4.49 (dd, J = 14.4, 5.4 Hz, 1H), 4.41-4.29 (m, 2H), 4.20-4.12 (m, 2H), 4.06-3.98 (m, 2H), 3.83- 3.71 (m, 2H), 3.45 (d, J = 14.3 Hz, 1H), 3.29- 3.23 (m, 1H), 3.16-3.11 (m, 1H), 3.09 (d, J = 4.7 Hz, 6H), 2.84 (dd, J = 17.9, 4.0 Hz, 1H), 2.70- 2.61 (m, 2H), 2.36 (dd, J = 14.5, 11.8 Hz, 2H), 2.20-2.14 (m, 1H), 1.95- 1.89 (m, 1H), 1.82 (dt, J = 7.6, 4.7 Hz, 1H) |

### Assays & Activity Data

KRAS G12D or G12V and KRAS G12V enzyme assays were carried out as follows:

### KRAS G12D or G12V and Wild-Type KRAS-In-Vitro RAS-RAF Binding Assay (RRB)

Biotinylated KRAS protein amino acids 1-169 (produced at Erasca) was labeled with streptavidin-terbium (lanthanide cryptate donor fluorophore) in assay buffer (50 mM HEPES, pH 7.5, 100 mM NaCl, 1 mM MgCl₂, 1 mM DTT) at a final concentration of 30 nM. In a separate reaction mixture, 30 nM cRAF (RBD) (Abcam, Cambridge MA) was labeled with anti-GST d2 (acceptor fluorophore). Labeling reactions were incubated for one hour at room temperature.

Compounds of interest were incubated with the labeled-KRAS for 60 minutes at room temperature at a final DMSO concentration of 5% in a black ½ area microtiter plate (50 μL final reaction volume). Following the compound incubation, SOS1 catalytic domain (produced at Erasca) and GTPgS were added to the reaction to initiate nucleotide exchange (60 minutes exchange reaction). Once in the GTP state KRAS will bind to cRAF. No binding will occur if KRAS remains in the GDP state. Compounds may block nucleotide exchange or may create a steric obstruction to cRAF-KRAS interaction by binding to the RAS effector site.

Following the exchange reaction, the labeled KRAS and cRAF were mixed in equal volume (30 μL each) and incubated for 30-60 minutes at room temperature. A portion of this mixture was transferred to a white 384-well plate (20 μL per well in duplicate) and read on an HTRF compatible plate reader (ClarioSTAR). Fluorescent resonance energy transfer (FRET) was measured at equilibrium. FRET signal will be high if KRAS-cRAF binding occurs. FRET signal will be low if KRAS-cRAF binding is inhibited by the test compound. The results for exemplary compounds are shown below in Table 2.

TABLE 2
Inhibition of RAS-RAF Binding (in-vitro)

| | KRAS (G12V) | KRAS (G12D) |
|---|---|---|
| Compound | IC₅₀ (nM) | IC₅₀ (nM) |
| Example 1 | 17.52 | 10.06 |

### Inhibition of KRAS Codon 12 Mutant-Mediated Phospho-ERK1/2 Inhibition by Exemplary Compounds of Formula

KRAS G12D or G12V and G12V mutant cell lines, AsPC-1 cells (ATCC CRL-1682), and SW-620 (ATCC CCL-227), respectively, were cultured according to published protocols and maintained at 37° C. in either 5% CO₂ for AsPC-1 or no CO₂ for SW-620. The phospho-ERK HTRF assay was executed following provider's protocol (CisBio #64AERPEH). AsPC-1 or SW-620 were plated at a density of 50,000 cells per well in a 96-well plate (Coming #3903) in respective medias (for AsPC-1, RPMI+10% FBS+1% Pen/Strep, and for SW-620, L-15+10% FBS+1% Pen/Strep) and maintained at 37° C. in either 5% CO₂ for AsPC-1 or no CO₂ for SW-620. Cells were allowed to adhere overnight and treated the following day with a Tecan D300e Digital Dispenser (Tecan Group Ltd., Switzerland) using an 11-point dose response starting at 2,500 nM of exemplified compounds followed by sequential 1:3 dilutions for 4 hours. Following compound treatment, the cells were washed once with ice-cold PBS. Cells were lysed by adding 50 μL of lysis buffer (lx) supplemented with lx Pierce Halt Protease and Phosphatase inhibitor and incubated for 60 minutes at 4° C. with shaking. After lysis, 16 μL of cell lysate from the 96-well cell-culture plate was transferred to a 384-well plate (Perkin Elmer #6007290). The premixed antibody solution was prepared by mixing (vol/vol) advanced phospho-ERK1/2 d2 antibody and advanced phospho-ERK1/2 Eu Cryptate antibody. The premixed antibody solution (4 μL) was added to the detection plate containing cell lysate. The detection plate was incubated overnight at 4° C., the HTRF signal was read the next day by using either a Spectramax M5 or Spectramax i3 microplate reader (Molecular Devices, San Jose, CA, USA), and data was processed according to manufacturer's protocol, and shown in Table 3.

TABLE 3
pERK Inhibition

| | AsPC-1 | SW-620 |
|---|---|---|
| Compound | IC₅₀ (nM) | IC₅₀ (nM) |
| Example 1 | 12.79 | 14.44 |

Although the foregoing embodiments have been described in some detail by way of illustration and Example for purposes of clarity of understanding, one skilled in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula (I-1) or Formula (I-2): 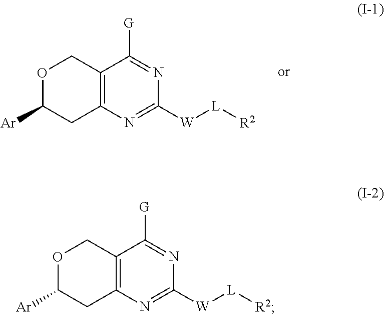
or a stereoisomer, a tautomer, or a pharmaceutically acceptable salt thereof, wherein:
G is 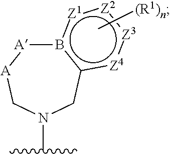
Ar is aryl or heteroaryl;
wherein Ar is optionally substituted with 1 to 5 groups selected from —OH, halo, C₁₋₃ alkyl, C₂-C₄ alkynyl, C₁₋₃ haloalkyl, C₃₋₆ cycloalkyl, a fused C₃₋₆ cycloalkyl, —CN, —CD₃, —SF₅ and —NH₂;
A is —CF₂, —CR^{7a}R^{7b}, or bond;
A′ is —CH₂—, —O—, —NH—, —S—, and —C═O;
B is C or N;
W is —O—, —NR³, —S—, 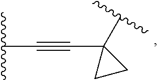 [SMILES: *C#CC1(*)CC1]
or absent;
L is C₁₋₃ alkyl or absent;
each of Z¹, Z², Z³, Z⁴ is independently null, CR¹, CH, 0, N(R′), NH, N, or S;
wherein Z¹, Z², Z³, Z⁴ combine to form a 5- or 6-membered aromatic or heteroaromatic ring;
each R¹ is an optional substitution and is independently halo, C₁₋₆ alkyl, —OC₁-C₆, —(CH₂)ₙ—N(R^{1b})(R^{1c}), —C(O)R^{1a}, —C(O)—N(R^{1b})(R^{1c}), —C(═NH)N(R^{1b})(R^{1c}), —C(═S)N(R^{1b})(R^{1c}), —NH—(CH₂)ₙ—(SO)ₚR^{1a}, —S(O)ₚR^{1a}, —S(O)ₚ(R^{1b})(R^{1c}), —S(O)ₚN(R^{1b})(R^{1c}), —P(O)(R^{1b})(R^{1c}), C₃₋₆ cycloalkyl, 5- or 6-membered heteroaromatic ring or 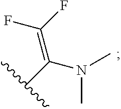 [SMILES: *C(=C(F)F)N(C)C]
or
two adjacent Ri combine to form a 5- to 7-membered aryl, 5- to 7-membered heteroaryl, 3- to 6-membered carbocylic, or 4- to 6-membered heterocyclic ring, any of which are optionally substituted with OH, amino, —C(O)N(R^{1b})(R^{1c}), halo, CN, CF₃, C₁₋₄ alkyl, C₁₋₄ alkoxy, and C₁₋₃ hydroxyalkyl; wherein the 5- to 7-membered heteroaryl or 4- to 6-membered heterocyclic ring comprises carbon atoms and 1-4 groups selected from halo, —O—, C═O, —S(O)ₚ, —S(O)ₚNH—, —NH—, and —N(C₁₋₄ alkyl);
R^{1a} is selected from halo, C₁₋₆ alkyl, C₁₋₃ hydroxyalkyl, C₁₋₃ haloalkyl, and 4- to 6-membered heterocyclyl;
R^{1b} and R^{1c} are each independently selected from hydrogen, CD₃, C₁₋₆ alkyl, —(CH₂)ₙOH, —(CH₂)ₙOR^{1a}, —(CH₂)ₙCN, and C₁₋₃ haloalkyl; or
R^{1b} and R^{1c} combine to form a 5- to 7-membered aryl, 5- to 7-membered heteroaryl, 3- to 6-membered carbocyclic, or 4- to 10-membered heterocyclic ring, wherein the 5- to 7-membered heteroaryl or 4- to 10-membered heterocyclic ring comprises carbon atoms and 1-4 groups selected from —O—, C═O, S(O)ₚ, S(O)ₚNH, —NH—, and —N(C₁₋₄ alkyl), wherein the 5-to 7-membered aryl, 5- to 7-membered heteroaryl, 3- to 6-membered carbocyclic, or 4- to 10-membered heterocyclic rings are optionally substituted with halo, —OH, C₁₋₄ alkyl, C₁₋₃ haloalkyl, C₁₋₄ alkoxy, —(CH₂)ₙN(R^{1d})(R^{1e}), C₁₋₄ hydroxyalkyl, —(CH₂)ₙOR^{1a}, or —C(O)N(R^{1d})(R^{1c});
R^{1d} and R^{1e} are each independently selected from hydrogen, CD₃, and C₁₋₆ alkyl;
wherein when R¹ is aryl or heteroaryl, Ri is optionally substituted with —OH, halo, C₁₋₃ alkyl, C₂-C₄ alkynyl, C₁₋₃ haloalkyl, —CN, —NH₂, or combinations thereof;
R² is 4- to 10-membered heterocyclyl, 3- to 10-membered cycloalkyl, 6- to 10-membered aryl or 5- to 10-membered heteroaryl, wherein said heterocyclyl, cycloalkyl, aryl and heteroaryl are optionally substituted with deuterium, OH, halo, CN, CF₃, C₁₋₄ alkyl, C₁₋₄ alkoxy, C₂₋₄ alkenyl, C₁₋₄ haloalkenyl, —N(R^{1d})(R^{1e}), oxo, and C₁₋₃ hydroxyalkyl;
R^{7a} and R^{7b} are independently selected from hydrogen and C₁₋₄ alkoxy, or R^{7a} and R^{7a} may optionally be taken together with the atom to which they are attached to form a 4- to 6-membered spirocyclic ring wherein any one of the carbon atoms may be replaced by —O—, —S—, or —NH—;
n, at each occurrence, is an integer independently selected from 0, 1, 2, 3, and 4; and,
p, at each occurrence, is an integer independently selected from 0, 1, and 2.

2. The compound of claim 1, wherein the compound is of Formula (I-1): 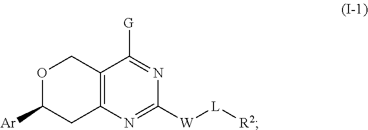
or a stereoisomer, a tautomer, a pharmaceutically acceptable salt thereof, wherein:
G is 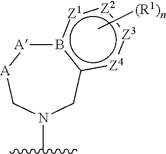
Ar is aryl or heteroaryl;
wherein Ar is optionally substituted with —OH, halo, C₁₋₃ alkyl, C₂-C₄ alkynyl, C₁₋₃ haloalkyl, —CN, CF₃, —NH₂, or combinations thereof;
A is —CH₂, —CF₂, or bond;
A′ is —CH₂, O, NH, O, S, and —C═O;
B is C or N;
each of Z¹, Z², Z³, Z⁴ is independently null, CR¹, O, N(R¹), NH, or S;
wherein of Z¹, Z², Z³, Z⁴ combine to form a 5- or 6-membered aromatic ring;
each is independently CH or N;
W is O, NR³, S, or absent;
L is C₁₋₃ alkyl or absent;
each R¹ is independently N-alkylamido, N,N-dialkylamido, halo, C₁₋₆ alkyl or C₃₋₆ cycloalkyl, aryl, or heteroaryl, wherein when R¹ is aryl or heteroaryl, R¹ is optionally substituted with —OH, halo, C₁₋₃ alkyl, C₂-C₄ alkynyl, C₁₋₃ haloalkyl, —CN, CF₃, —NH₂, or combinations thereof; or
two R¹ combine to form a 5- to 7-membered aryl, heteroaryl, carbocylic, or heterocyclic ring, any of which are optionally substituted with OH, amino, N-alkylamido, N,N-dialkylamido, halo, CN, CF₃, C₁₋₄ alkyl, C₁₋₄ alkoxy, and C₁₋₃ alkyl-OH;
R² is 4-10 membered heterocyclyl, 3-10 membered cycloalkyl, 6-10 membered aryl or 5-10 membered heteroaryl, wherein said heterocyclyl, cycloalkyl, aryl and heteroaryl are optionally substituted with deuterium, OH, halo, CN, CF₃, C₁₋₄ alkyl, C₁₋₄ alkoxy, and C₁₋₃ alkyl-OH;
and,
n is 0, 1, 2, 3, or 4.

3. The compound of claim 1, wherein the compound is of Formula (Ia):

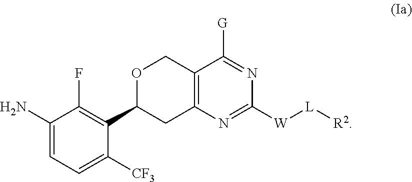

4. The compound of claim 1, wherein the compound is of Formula (Ib):

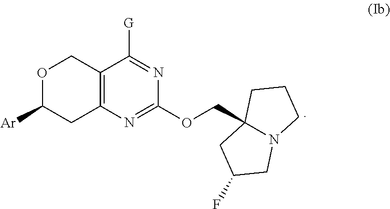

5. The compound of claim 1, wherein the compound is of Formula (Ic):

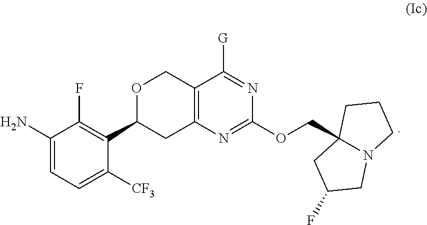

6. The compound of claim 1, wherein the compound is of Formula (Id): 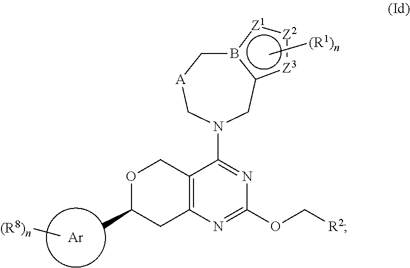
or a stereoisomer, a tautomer, or a pharmaceutically acceptable salt thereof,
wherein:
Ar is phenyl or pyridine;
each R⁸ is independently selected from NH₂, OH, CH₃, F, Cl, Br, I, CF₃ and CD₃;
Z¹ is O or N,
Z² is CR¹, CH, O, N(R¹), NH, or N;
Z³ is CR¹, CH, O, N(R¹), NH, N, or S; and,
n, at each occurrence, is an integer independently selected from 2, 3 and 4.

7. The compound of claim 1 or 4 wherein Ar is: 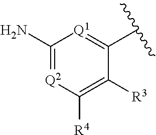
wherein Q¹ and Q² are independently CR⁷ or N; and,
each R³, R⁴, R⁵ and R⁷ are independently selected from H, CH₃, F, Cl, Br, I, CF₃ and CD₃.

8. The compound of claim 1 or 4, wherein Ar is: 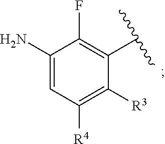
wherein R³ is H, CH₃, CF₃, Br, Cl, or CD₃; and
R⁴ is H, CH₃, CF₃, Cl, Br, or I.

9. The compound of claim 1 or 4, wherein Ar is: 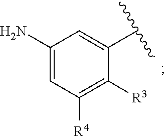
wherein R³ is H, CH₃, CF₃, Br, Cl, or CD₃; and
R⁴ is H, CH₃, CF₃, Cl, Br, or I.

10. The compound of claim 1 or 4, wherein Ar is selected from:

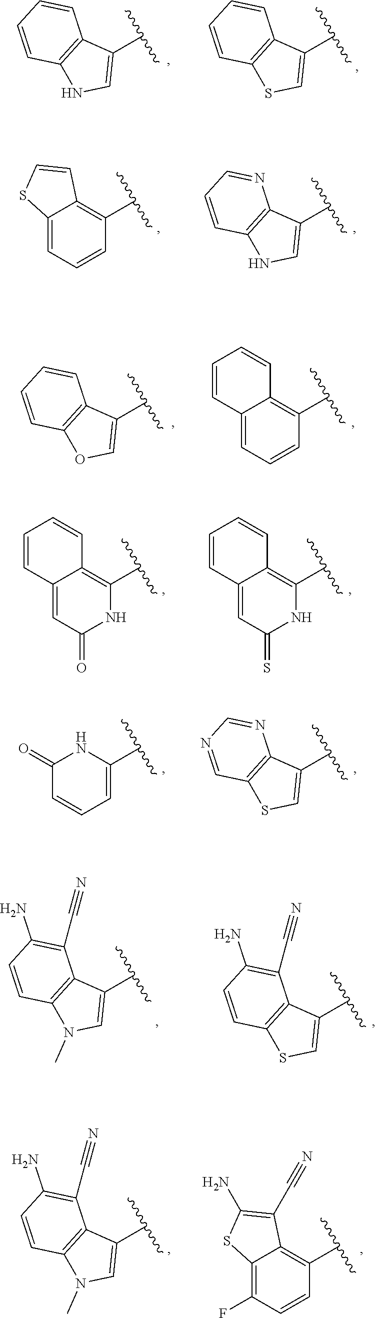

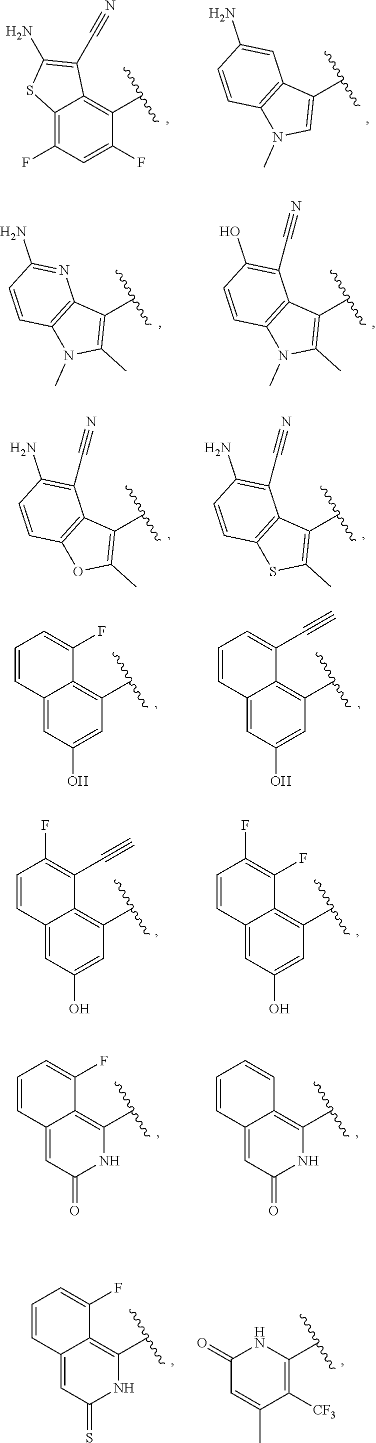

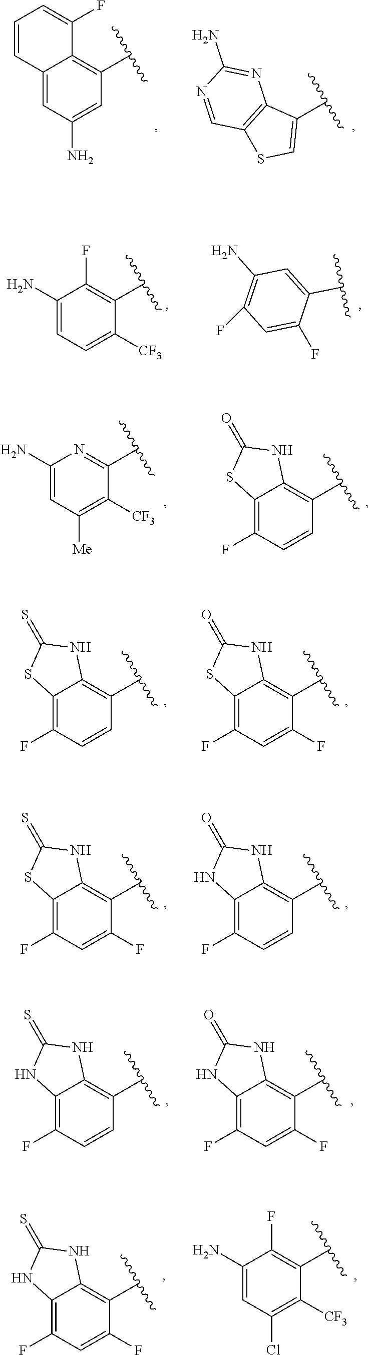

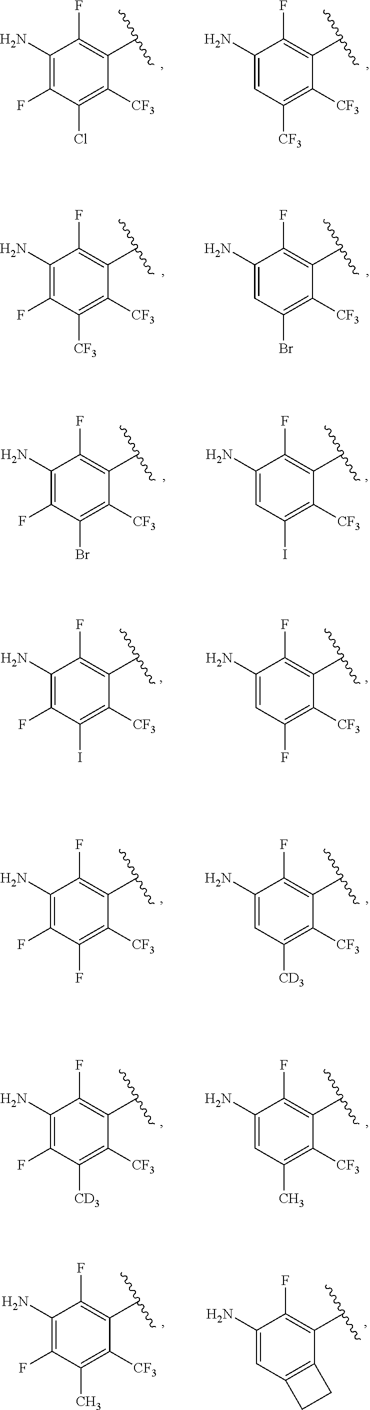

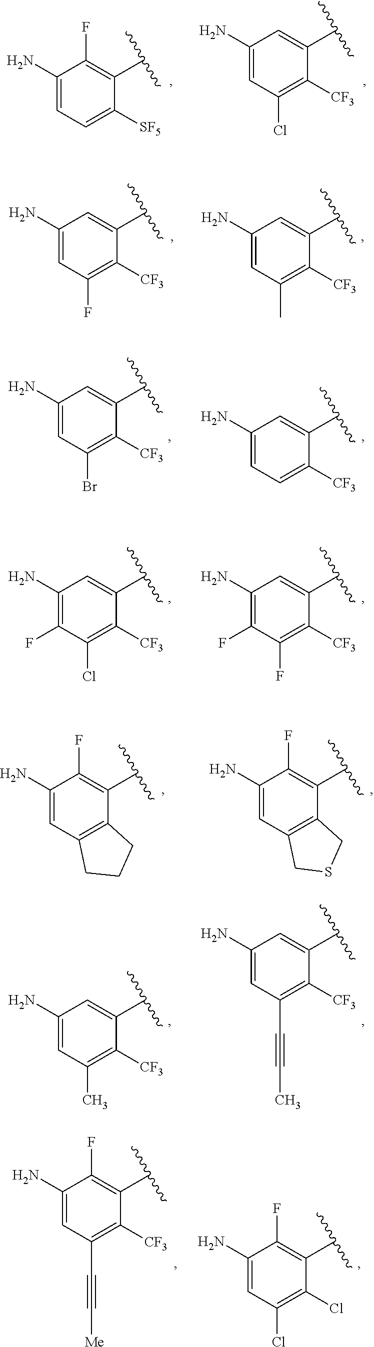

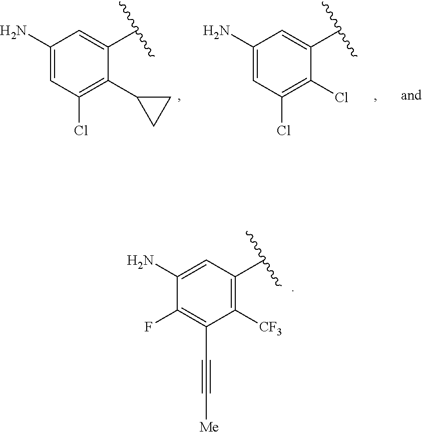 [SMILES: *c1cc(N)cc(Cl)c1C1CC1 ; *c1cc(N)cc(Cl)c1Cl ; *c1cc(N)c(F)c(C#CC)c1C(F)(F)F]

11. The compound of claim 1 or 3, wherein W-L-R² is selected from: 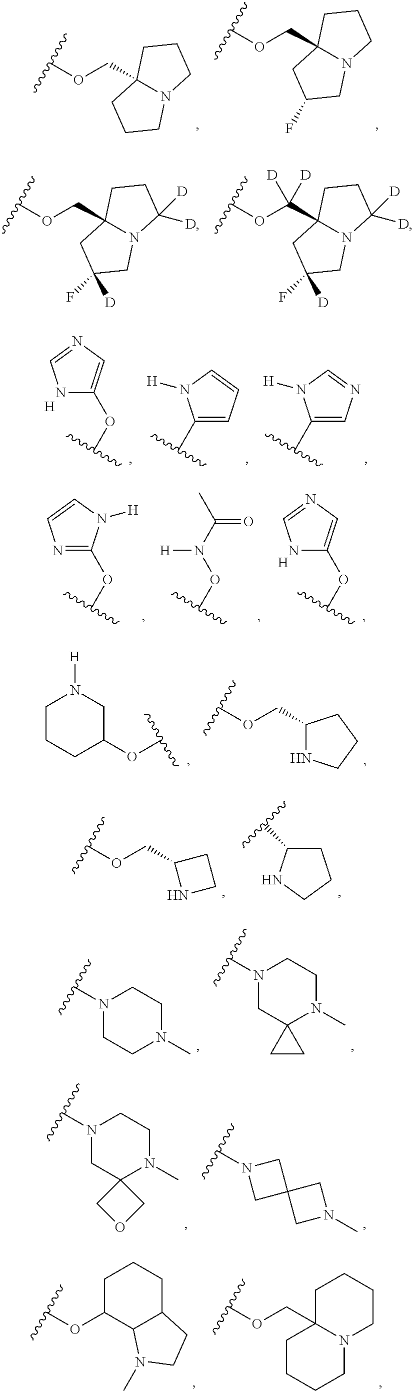 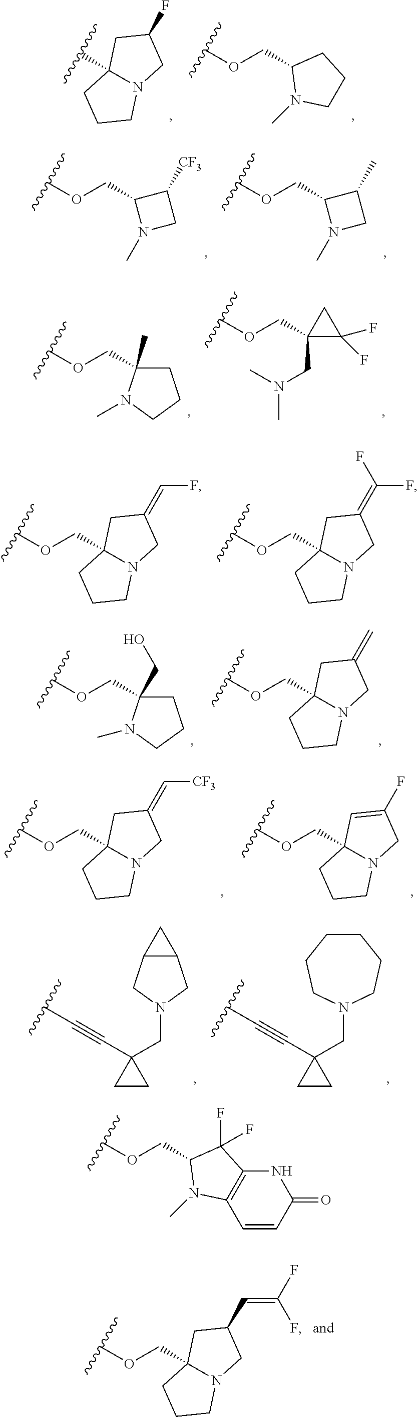 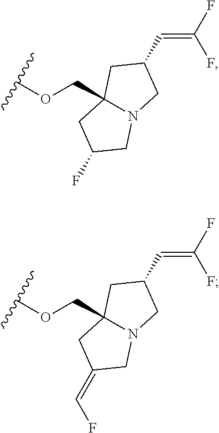 [SMILES: *OC[C@]12C[C@@H](F)CN1C[C@@H](C=C(F)F)C2 ; *OC[C@]12C/C(=C/F)CN1C[C@@H](C=C(F)F)C2]
wherein each are optionally substituted with OH, halo, CN, CF₃, C₁₋₄ alkyl, C₁₋₄ alkoxy, and C₁₋₃ hydroxyalkyl.

12. The compound of any one of claims 1 to 11, wherein G is: 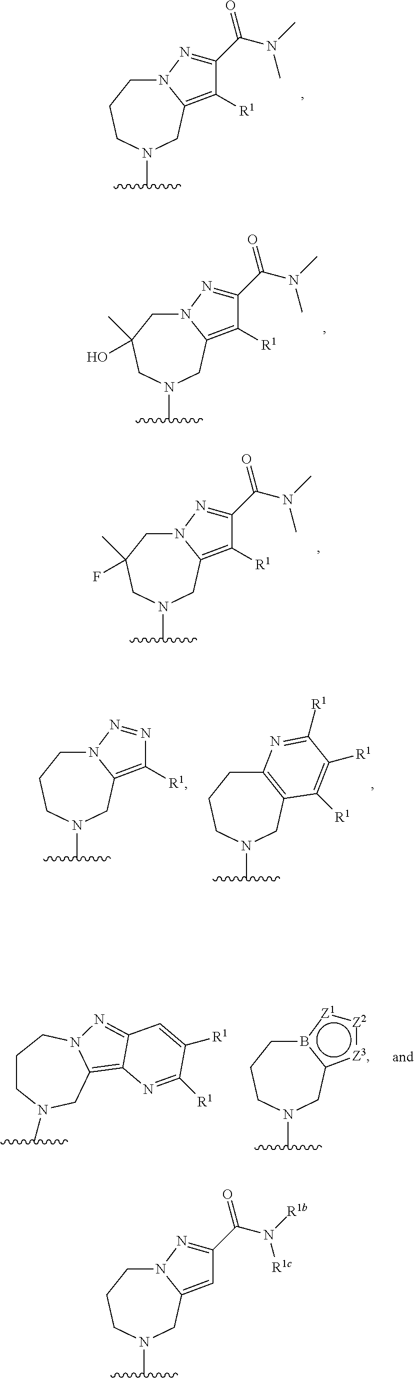
wherein Z¹, Z², Z³ are each independently selected from CR¹, CH, O, N(R¹), NH, N, and S.

13. The compound of any one of claims 1 to 10, wherein G is selected from:

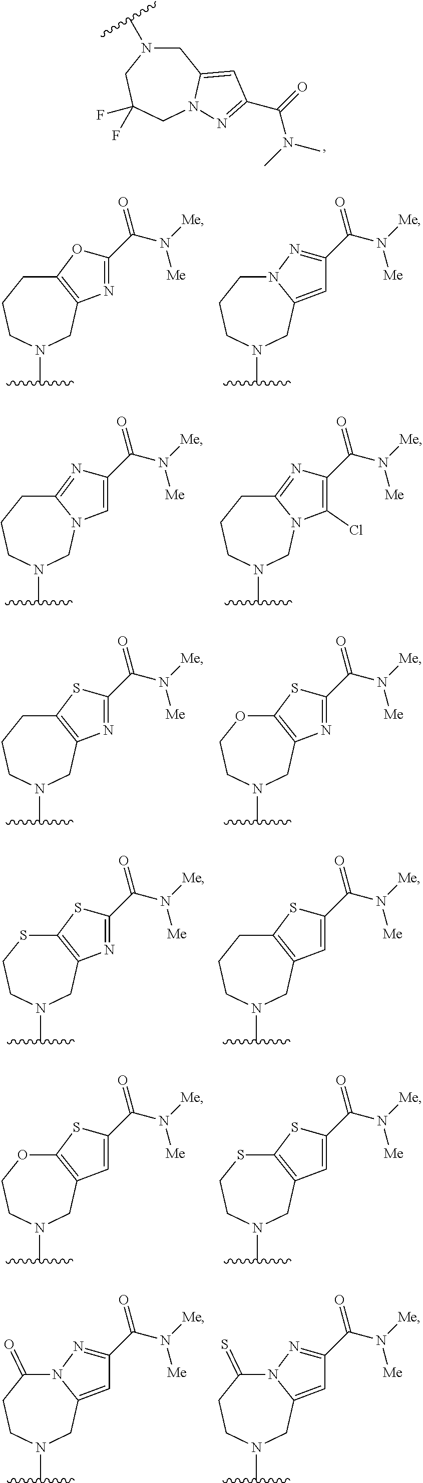

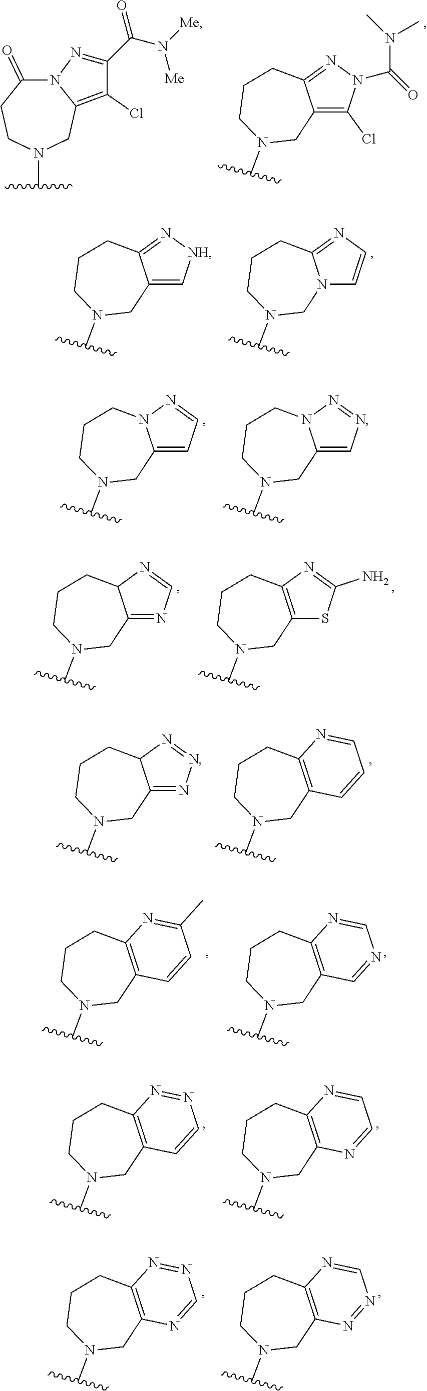

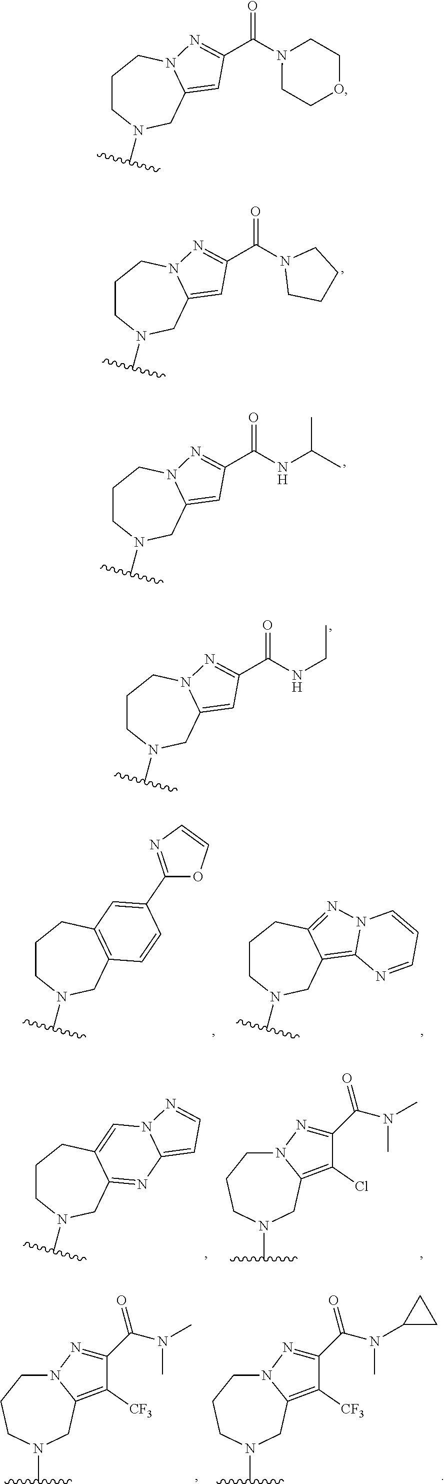

14. The compound of claim 1 selected from: 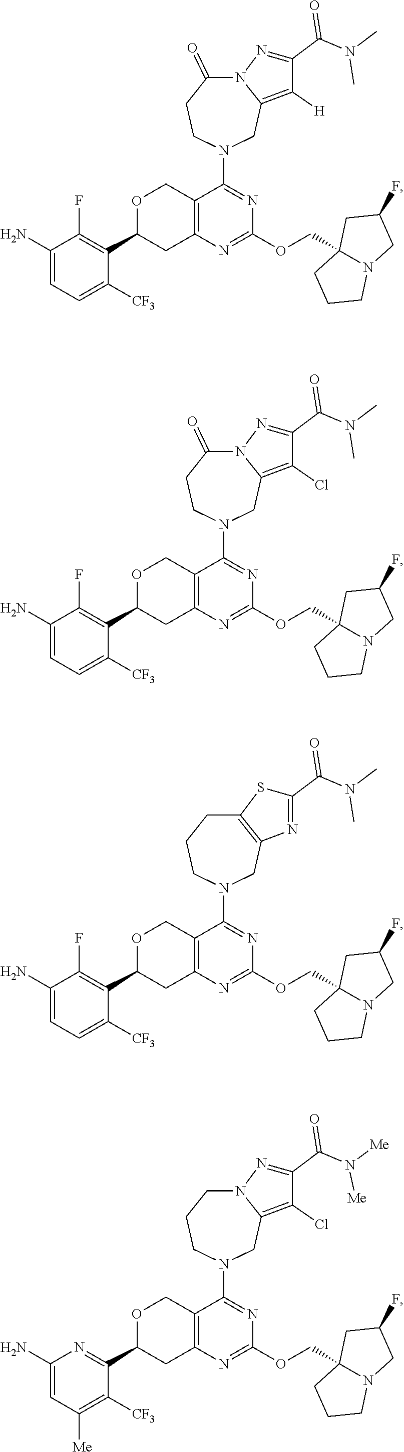 [SMILES: CN(C)C(=O)c1cc2n(n1)C(=O)CCN(c1nc(OC[C@@]34CCCN3C[C@H](F)C4)nc3c1CO[C@H](c1c(C(F)(F)F)ccc(N)c1F)C3)C2 ; CN(C)C(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2=O ; CN(C)C(=O)c1nc2c(s1)CCCN(c1nc(OC[C@@]34CCCN3C[C@H](F)C4)nc3c1CO[C@H](c1c(C(F)(F)F)ccc(N)c1F)C3)C2 ; Cc1cc(N)nc([C@@H]2Cc3nc(OC[C@@]45CCCN4C[C@H](F)C5)nc(N4CCCn5nc(C(=O)N(C)C)c(Cl)c5C4)c3CO2)c1C(F)(F)F] 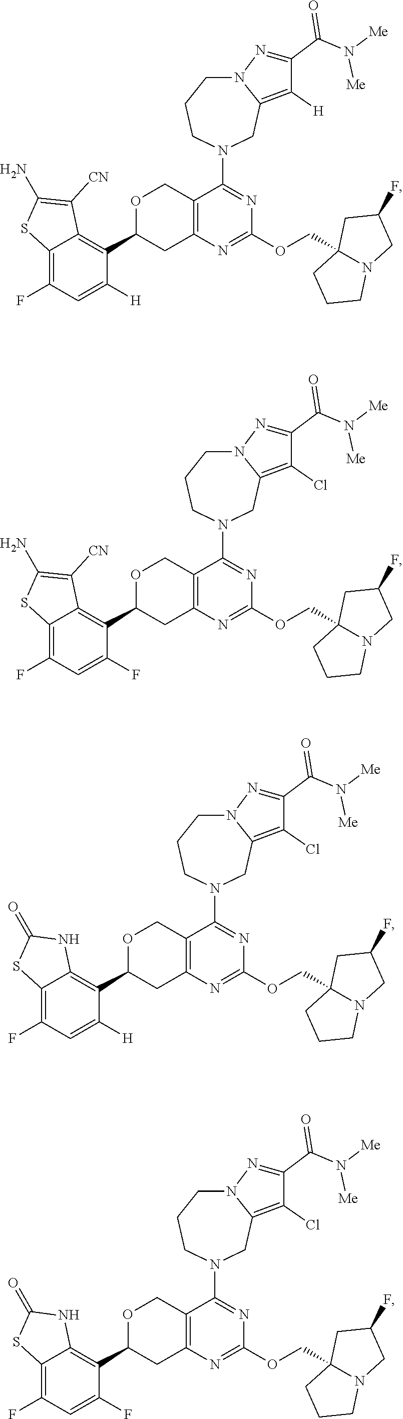 [SMILES: CN(C)C(=O)c1cc2n(n1)CCCN(c1nc(OC[C@@]34CCCN3C[C@H](F)C4)nc3c1CO[C@H](c1ccc(F)c4sc(N)c(C#N)c14)C3)C2 ; CN(C)C(=O)c1nn2c(c1Cl)CN(c1nc(OC[C@@]34CCCN3C[C@H](F)C4)nc3c1CO[C@H](c1c(F)cc(F)c4sc(N)c(C#N)c14)C3)CCC2 ; CN(C)C(=O)c1nn2c(c1Cl)CN(c1nc(OC[C@@]34CCCN3C[C@H](F)C4)nc3c1CO[C@H](c1ccc(F)c4sc(=O)[nH]c14)C3)CCC2 ; CN(C)C(=O)c1nn2c(c1Cl)CN(c1nc(OC[C@@]34CCCN3C[C@H](F)C4)nc3c1CO[C@H](c1c(F)cc(F)c4sc(=O)[nH]c14)C3)CCC2] 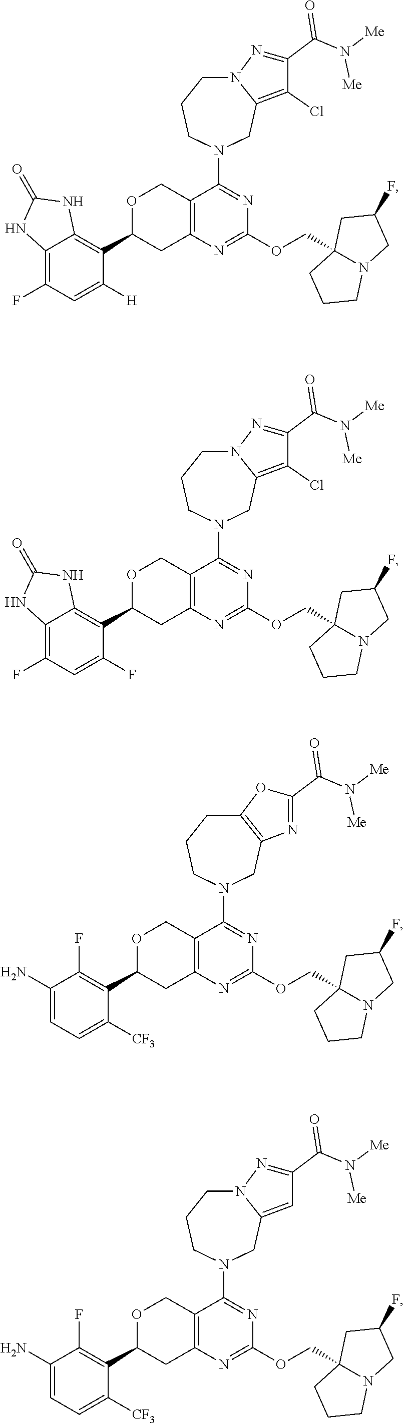 [SMILES: CN(C)C(=O)c1nn2c(c1Cl)CN(c1nc(OC[C@@]34CCCN3C[C@H](F)C4)nc3c1CO[C@H](c1ccc(F)c4[nH]c(=O)[nH]c14)C3)CCC2 ; CN(C)C(=O)c1nn2c(c1Cl)CN(c1nc(OC[C@@]34CCCN3C[C@H](F)C4)nc3c1CO[C@H](c1c(F)cc(F)c4[nH]c(=O)[nH]c14)C3)CCC2 ; CN(C)C(=O)c1nc2c(o1)CCCN(c1nc(OC[C@@]34CCCN3C[C@H](F)C4)nc3c1CO[C@H](c1c(C(F)(F)F)ccc(N)c1F)C3)C2 ; CN(C)C(=O)c1cc2n(n1)CCCN(c1nc(OC[C@@]34CCCN3C[C@H](F)C4)nc3c1CO[C@H](c1c(C(F)(F)F)ccc(N)c1F)C3)C2] 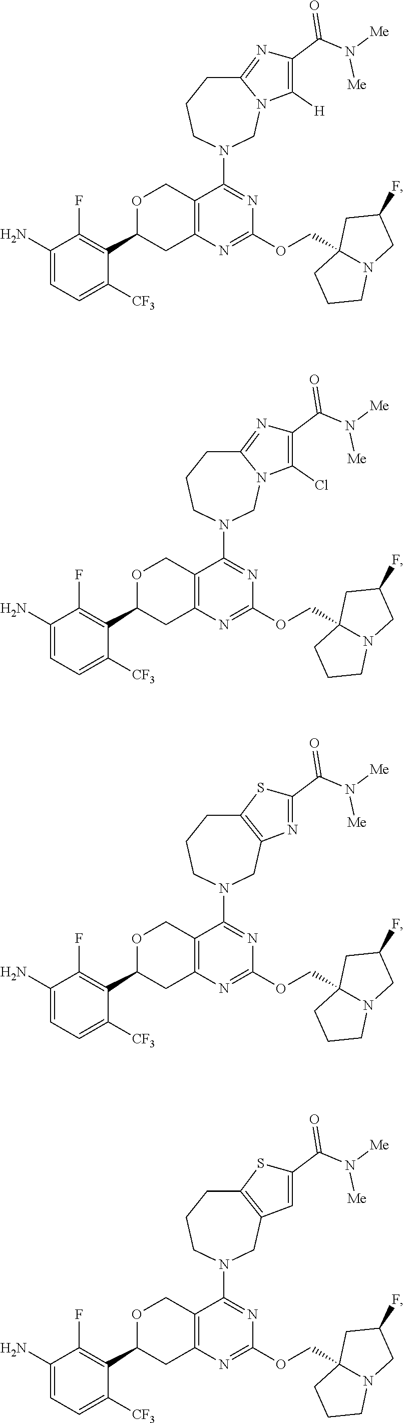 [SMILES: CN(C)C(=O)c1cn2c(n1)CCCN(c1nc(OC[C@@]34CCCN3C[C@H](F)C4)nc3c1CO[C@H](c1c(C(F)(F)F)ccc(N)c1F)C3)C2 ; CN(C)C(=O)c1nc2n(c1Cl)CN(c1nc(OC[C@@]34CCCN3C[C@H](F)C4)nc3c1CO[C@H](c1c(C(F)(F)F)ccc(N)c1F)C3)CCC2 ; CN(C)C(=O)c1nc2c(s1)CCCN(c1nc(OC[C@@]34CCCN3C[C@H](F)C4)nc3c1CO[C@H](c1c(C(F)(F)F)ccc(N)c1F)C3)C2 ; CN(C)C(=O)c1cc2c(s1)CCCN(c1nc(OC[C@@]34CCCN3C[C@H](F)C4)nc3c1CO[C@H](c1c(C(F)(F)F)ccc(N)c1F)C3)C2] 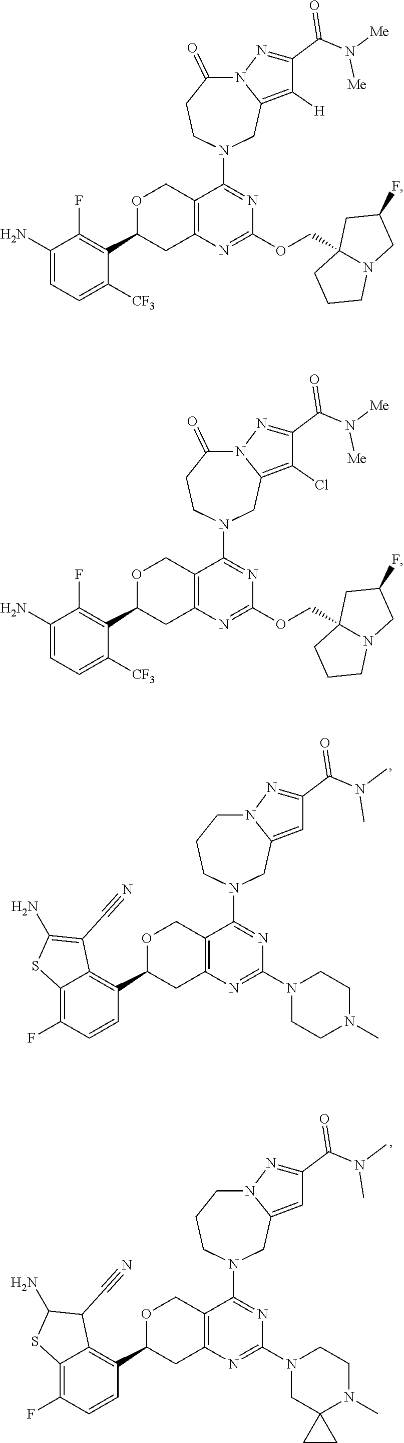 [SMILES: CN(C)C(=O)c1cc2n(n1)C(=O)CCN(c1nc(OC[C@@]34CCCN3C[C@H](F)C4)nc3c1CO[C@H](c1c(C(F)(F)F)ccc(N)c1F)C3)C2 ; CN(C)C(=O)c1nn2c(c1Cl)CN(c1nc(OC[C@@]34CCCN3C[C@H](F)C4)nc3c1CO[C@H](c1c(C(F)(F)F)ccc(N)c1F)C3)CCC2=O ; CN1CCN(c2nc3c(c(N4CCCn5nc(C(=O)N(C)C)cc5C4)n2)CO[C@H](c2ccc(F)c4sc(N)c(C#N)c24)C3)CC1 ; CN(C)C(=O)c1cc2n(n1)CCCN(c1nc(N3CCN(C)C4(CC4)C3)nc3c1CO[C@H](c1ccc(F)c4c1C(C#N)C(N)S4)C3)C2] 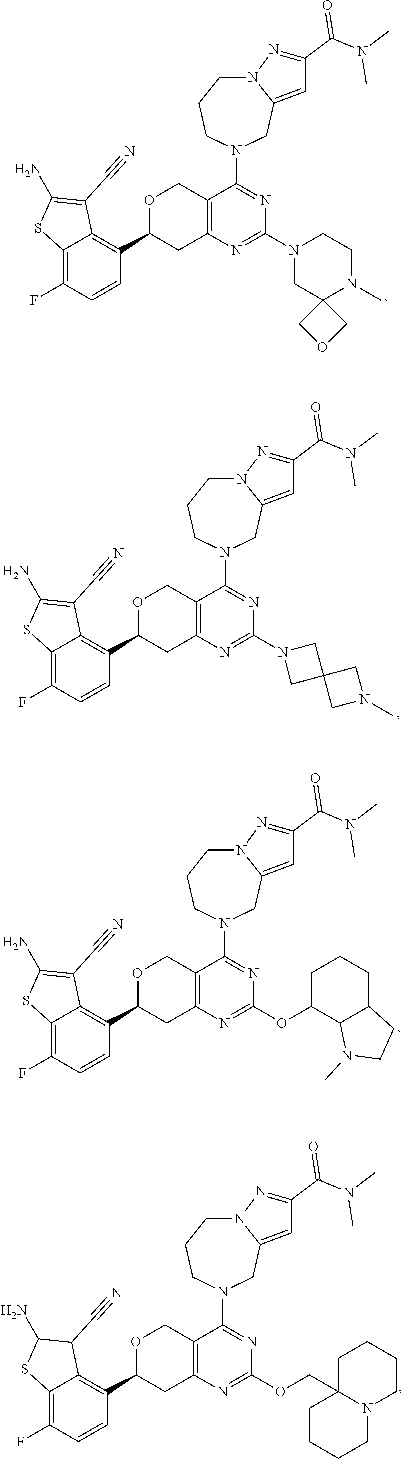 [SMILES: CN(C)C(=O)c1cc2n(n1)CCCN(c1nc(N3CCN(C)C4(COC4)C3)nc3c1CO[C@H](c1ccc(F)c4sc(N)c(C#N)c14)C3)C2 ; CN1CC2(C1)CN(c1nc3c(c(N4CCCn5nc(C(=O)N(C)C)cc5C4)n1)CO[C@H](c1ccc(F)c4sc(N)c(C#N)c14)C3)C2 ; CN(C)C(=O)c1cc2n(n1)CCCN(c1nc(OC3CCCC4CCN(C)C43)nc3c1CO[C@H](c1ccc(F)c4sc(N)c(C#N)c14)C3)C2 ; CN(C)C(=O)c1cc2n(n1)CCCN(c1nc(OCC34CCCCN3CCCC4)nc3c1CO[C@H](c1ccc(F)c4c1C(C#N)C(N)S4)C3)C2] 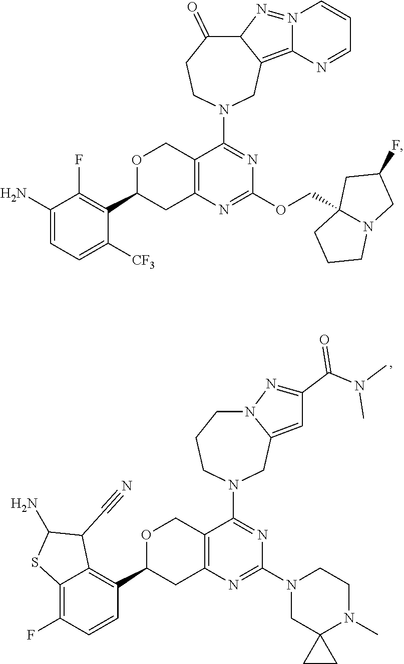 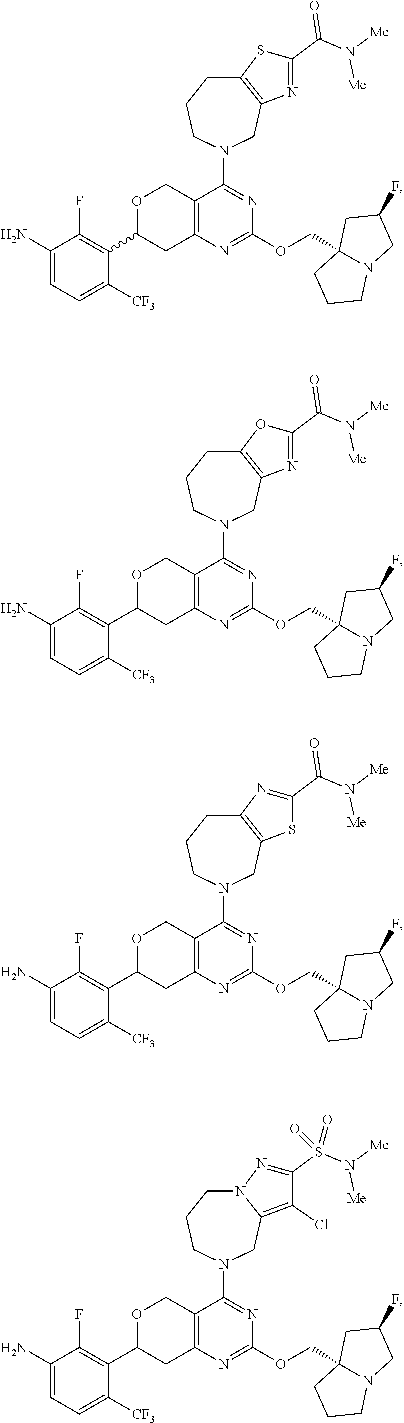 [SMILES: CN(C)C(=O)c1nc2c(s1)CCCN(c1nc(OC[C@@]34CCCN3C[C@H](F)C4)nc3c1COC(c1c(C(F)(F)F)ccc(N)c1F)C3)C2 ; CN(C)C(=O)c1nc2c(o1)CCCN(c1nc(OC[C@@]34CCCN3C[C@H](F)C4)nc3c1COC(c1c(C(F)(F)F)ccc(N)c1F)C3)C2 ; CN(C)C(=O)c1nc2c(s1)CN(c1nc(OC[C@@]34CCCN3C[C@H](F)C4)nc3c1COC(c1c(C(F)(F)F)ccc(N)c1F)C3)CCC2 ; CN(C)S(=O)(=O)c1nn2c(c1Cl)CN(c1nc(OC[C@@]34CCCN3C[C@H](F)C4)nc3c1COC(c1c(C(F)(F)F)ccc(N)c1F)C3)CCC2] 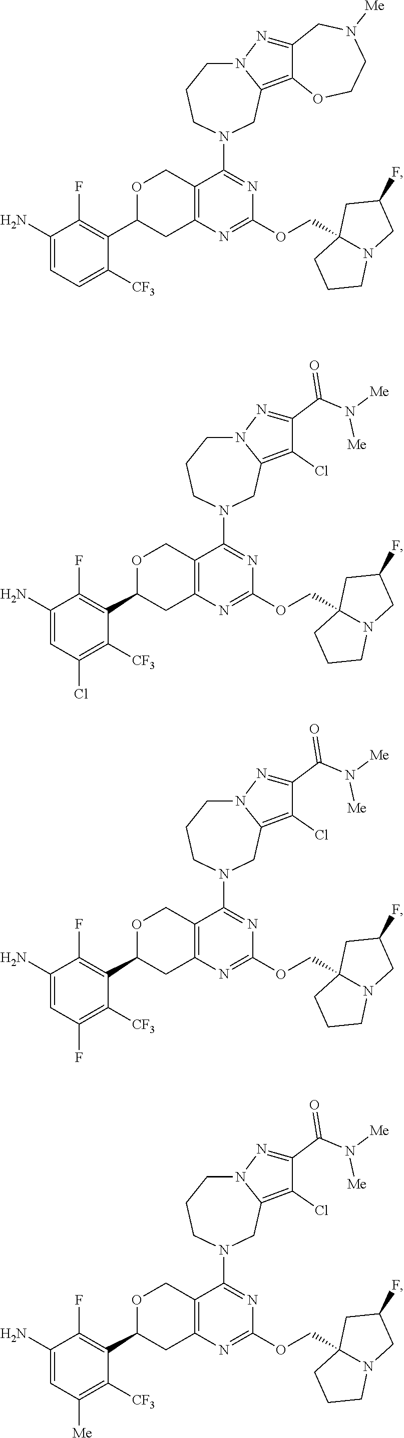 [SMILES: CN1CCOc2c(nn3c2CN(c2nc(OC[C@@]45CCCN4C[C@H](F)C5)nc4c2COC(c2c(C(F)(F)F)ccc(N)c2F)C4)CCC3)C1 ; CN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2 ; CN(C)C(=O)c1nn2c(c1Cl)CN(c1nc(OC[C@@]34CCCN3C[C@H](F)C4)nc3c1CO[C@H](c1c(F)c(N)cc(F)c1C(F)(F)F)C3)CCC2 ; Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F] 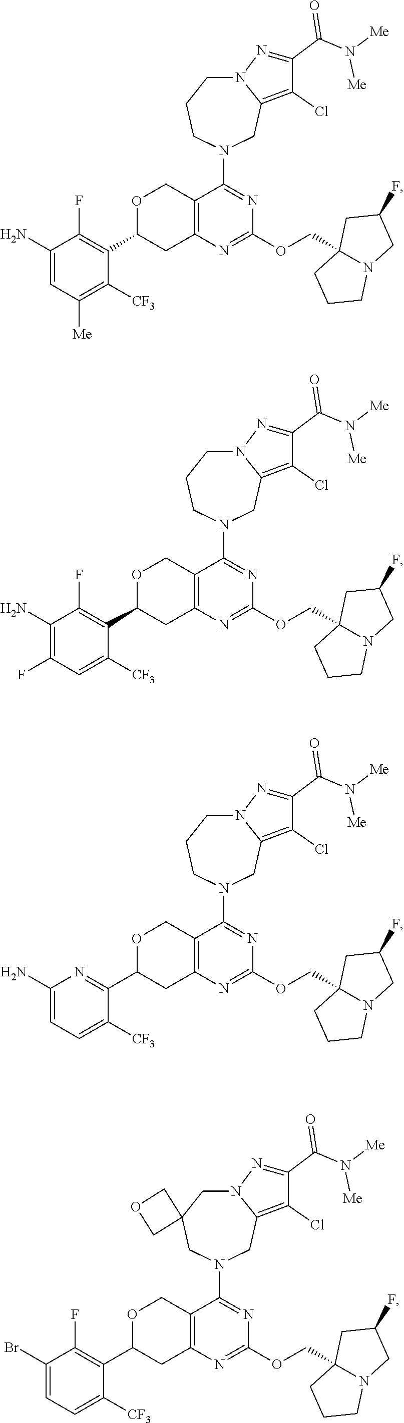 [SMILES: Cc1cc(N)c(F)c([C@H]2Cc3nc(OC[C@@]45CCCN4C[C@H](F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C[C@H](F)C4)nc3c1CO[C@H](c1c(C(F)(F)F)cc(F)c(N)c1F)C3)CCC2 ; CN(C)C(=O)c1nn2c(c1Cl)CN(c1nc(OC[C@@]34CCCN3C[C@H](F)C4)nc3c1COC(c1nc(N)ccc1C(F)(F)F)C3)CCC2 ; CN(C)C(=O)c1nn2c(c1Cl)CN(c1nc(OC[C@@]34CCCN3C[C@H](F)C4)nc3c1COC(c1c(C(F)(F)F)ccc(Br)c1F)C3)CC1(COC1)C2] 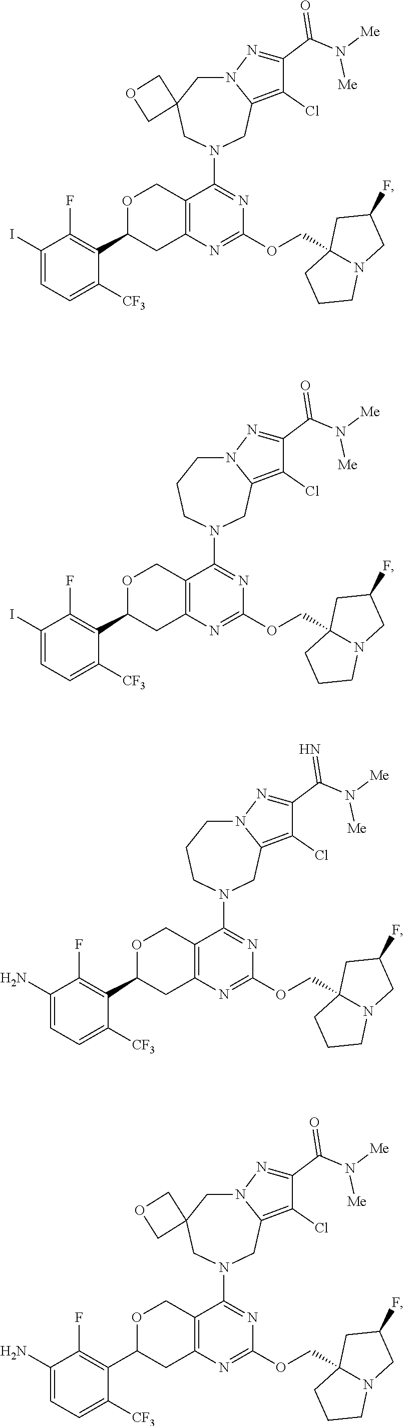 [SMILES: CN(C)C(=O)c1nn2c(c1Cl)CN(c1nc(OC[C@@]34CCCN3C[C@H](F)C4)nc3c1CO[C@H](c1c(C(F)(F)F)ccc(I)c1F)C3)CC1(COC1)C2 ; CN(C)C(=O)c1nn2c(c1Cl)CN(c1nc(OC[C@@]34CCCN3C[C@H](F)C4)nc3c1CO[C@H](c1c(C(F)(F)F)ccc(I)c1F)C3)CCC2 ; CN(C)C(=N)c1nn2c(c1Cl)CN(c1nc(OC[C@@]34CCCN3C[C@H](F)C4)nc3c1CO[C@H](c1c(C(F)(F)F)ccc(N)c1F)C3)CCC2 ; CN(C)C(=O)c1nn2c(c1Cl)CN(c1nc(OC[C@@]34CCCN3C[C@H](F)C4)nc3c1COC(c1c(C(F)(F)F)ccc(N)c1F)C3)CC1(COC1)C2] 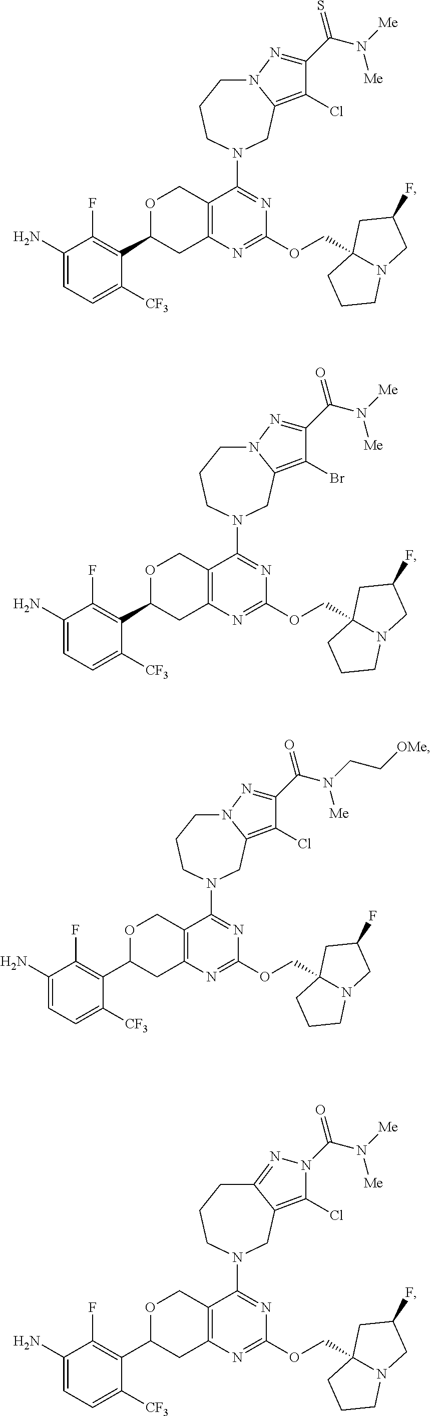 [SMILES: CN(C)C(=S)c1nn2c(c1Cl)CN(c1nc(OC[C@@]34CCCN3C[C@H](F)C4)nc3c1CO[C@H](c1c(C(F)(F)F)ccc(N)c1F)C3)CCC2 ; CN(C)C(=O)c1nn2c(c1Br)CN(c1nc(OC[C@@]34CCCN3C[C@H](F)C4)nc3c1CO[C@H](c1c(C(F)(F)F)ccc(N)c1F)C3)CCC2 ; COCCN(C)C(=O)c1nn2c(c1Cl)CN(c1nc(OC[C@@]34CCCN3C[C@H](F)C4)nc3c1COC(c1c(C(F)(F)F)ccc(N)c1F)C3)CCC2 ; CN(C)C(=O)n1nc2c(c1Cl)CN(c1nc(OC[C@@]34CCCN3C[C@H](F)C4)nc3c1COC(c1c(C(F)(F)F)ccc(N)c1F)C3)CCC2] 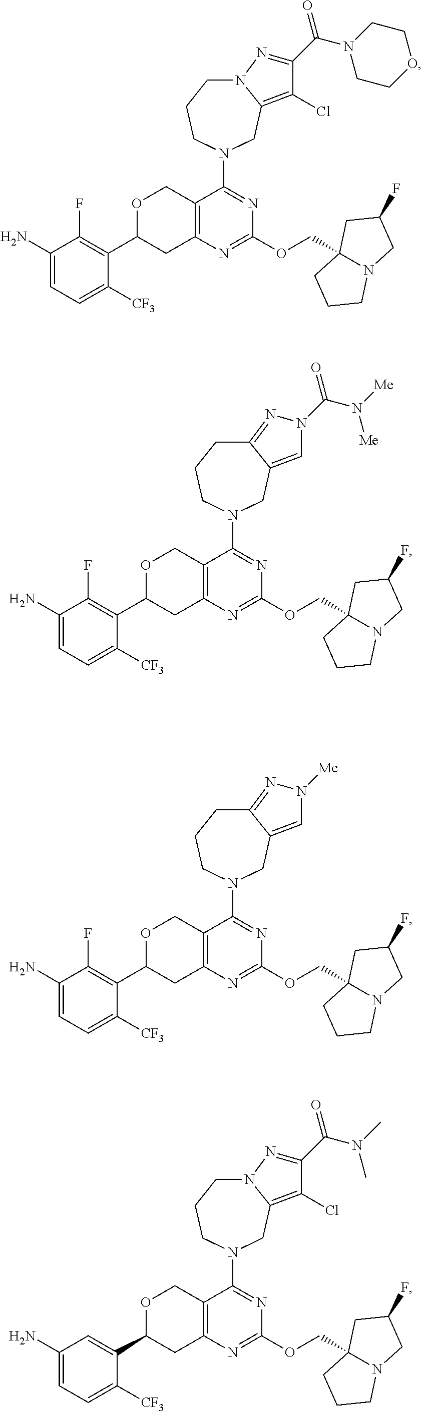 [SMILES: Nc1ccc(C(F)(F)F)c(C2Cc3nc(OC[C@@]45CCCN4C[C@H](F)C5)nc(N4CCCn5nc(C(=O)N6CCOCC6)c(Cl)c5C4)c3CO2)c1F ; CN(C)C(=O)n1cc2c(n1)CCCN(c1nc(OC[C@@]34CCCN3C[C@H](F)C4)nc3c1COC(c1c(C(F)(F)F)ccc(N)c1F)C3)C2 ; Cn1cc2c(n1)CCCN(c1nc(OC[C@@]34CCCN3C[C@H](F)C4)nc3c1COC(c1c(C(F)(F)F)ccc(N)c1F)C3)C2 ; CN(C)C(=O)c1nn2c(c1Cl)CN(c1nc(OC[C@@]34CCCN3C[C@H](F)C4)nc3c1CO[C@H](c1cc(N)ccc1C(F)(F)F)C3)CCC2] 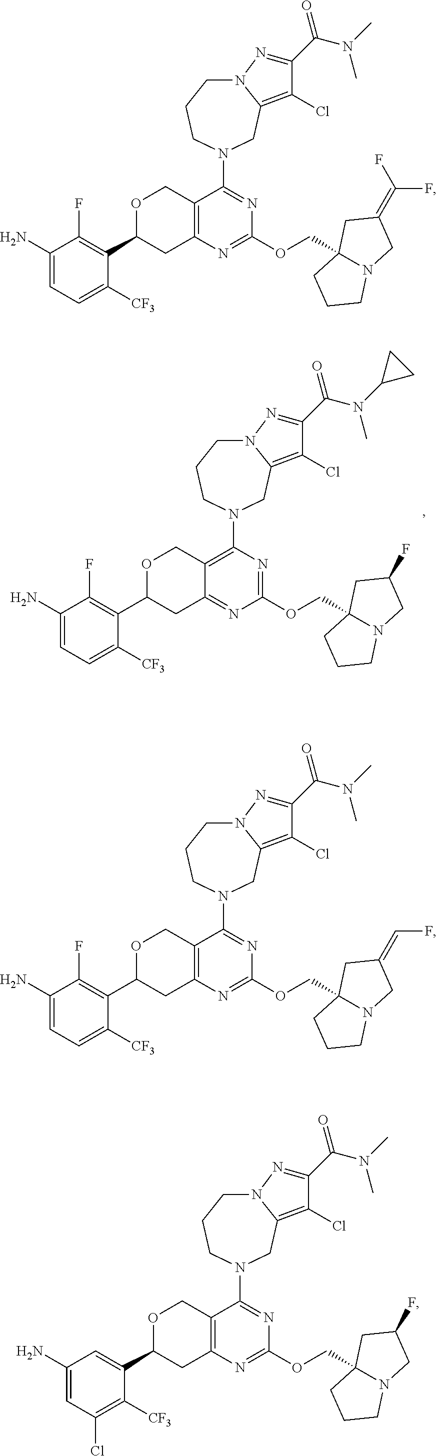 [SMILES: CN(C)C(=O)c1nn2c(c1Cl)CN(c1nc(OC[C@@]34CCCN3CC(=C(F)F)C4)nc3c1CO[C@H](c1c(C(F)(F)F)ccc(N)c1F)C3)CCC2 ; CN(C(=O)c1nn2c(c1Cl)CN(c1nc(OC[C@@]34CCCN3C[C@H](F)C4)nc3c1COC(c1c(C(F)(F)F)ccc(N)c1F)C3)CCC2)C1CC1 ; CN(C)C(=O)c1nn2c(c1Cl)CN(c1nc(OC[C@@]34CCCN3C/C(=C\F)C4)nc3c1COC(c1c(C(F)(F)F)ccc(N)c1F)C3)CCC2 ; CN(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2] 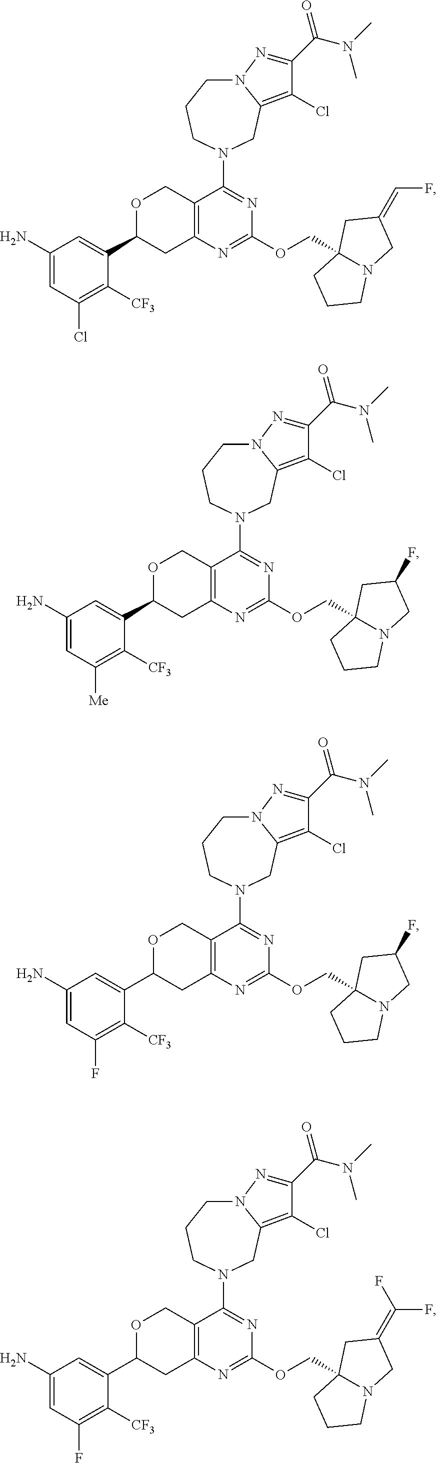 [SMILES: CN(C)C(=O)c1nn2c(c1Cl)CN(c1nc(OC[C@@]34CCCN3C/C(=C\F)C4)nc3c1CO[C@H](c1cc(N)cc(Cl)c1C(F)(F)F)C3)CCC2 ; Cc1cc(N)cc([C@@H]2Cc3nc(OC[C@@]45CCCN4C[C@H](F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C[C@H](F)C4)nc3c1COC(c1cc(N)cc(F)c1C(F)(F)F)C3)CCC2 ; CN(C)C(=O)c1nn2c(c1Cl)CN(c1nc(OC[C@@]34CCCN3CC(=C(F)F)C4)nc3c1COC(c1cc(N)cc(F)c1C(F)(F)F)C3)CCC2] 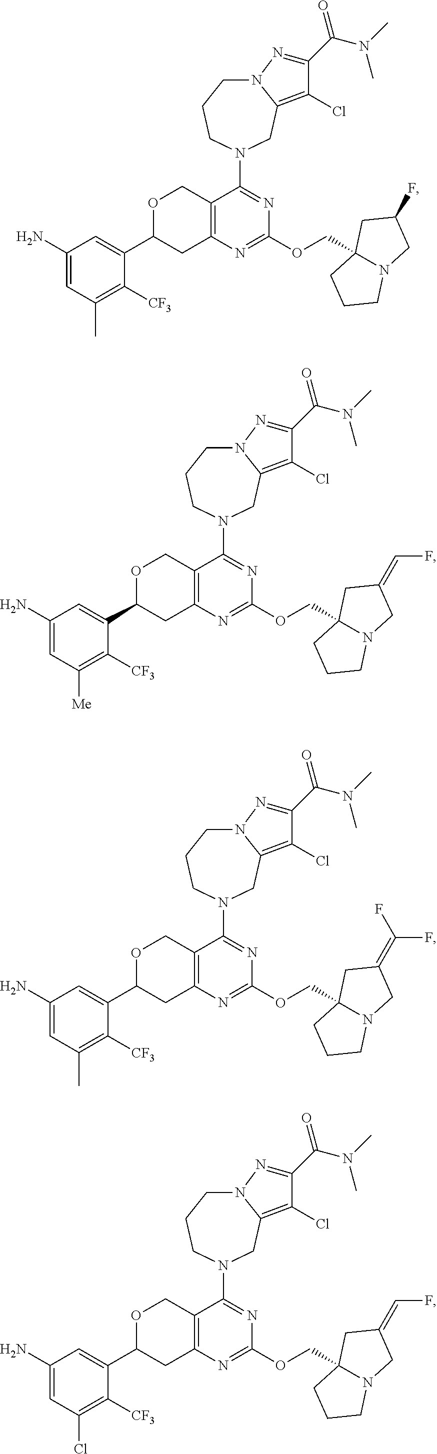 [SMILES: Cc1cc(N)cc(C2Cc3nc(OC[C@@]45CCCN4C[C@H](F)C5)nc(N4CCCn5nc(C(=O)N(C)C)c(Cl)c5C4)c3CO2)c1C(F)(F)F ; Cc1cc(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F ; Cc1cc(N)cc(C2Cc3nc(OC[C@@]45CCCN4CC(=C(F)F)C5)nc(N4CCCn5nc(C(=O)N(C)C)c(Cl)c5C4)c3CO2)c1C(F)(F)F ; CN(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2] 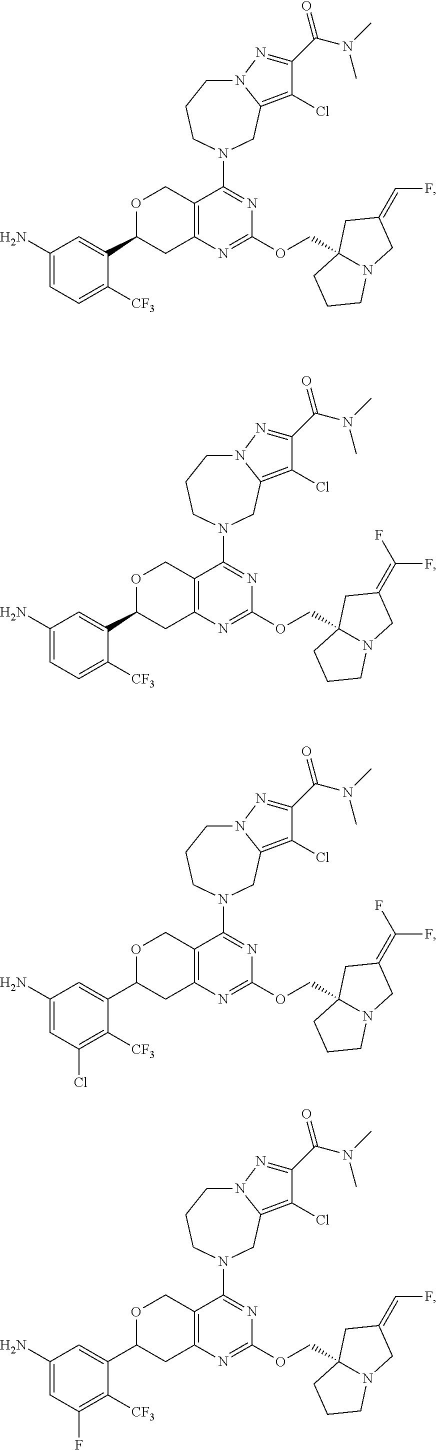 [SMILES: CN(C)C(=O)c1nn2c(c1Cl)CN(c1nc(OC[C@@]34CCCN3C/C(=C\F)C4)nc3c1CO[C@H](c1cc(N)ccc1C(F)(F)F)C3)CCC2 ; CN(C)C(=O)c1nn2c(c1Cl)CN(c1nc(OC[C@@]34CCCN3CC(=C(F)F)C4)nc3c1CO[C@H](c1cc(N)ccc1C(F)(F)F)C3)CCC2 ; CN(C)C(=O)c1nn2c(c1Cl)CN(c1nc(OC[C@@]34CCCN3CC(=C(F)F)C4)nc3c1COC(c1cc(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1COC(c1cc(N)cc(F)c1C(F)(F)F)C3)CCC2] 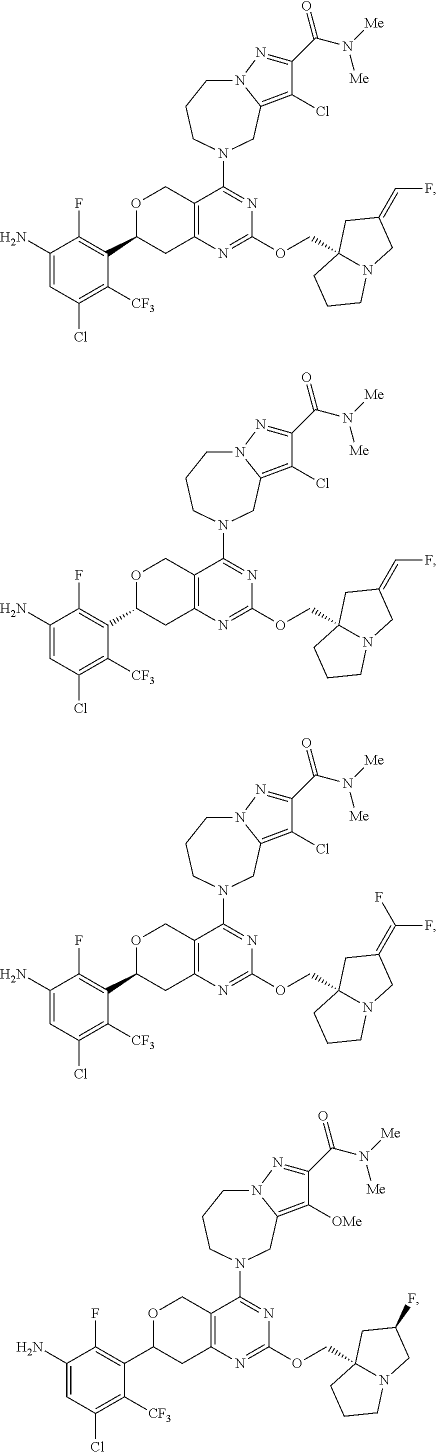 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1CO[C@@H](c1c(F)c(N)cc(Cl)c1C(F)(F)F)C3)CCC2 ; CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2 ; COc1c(C(=O)N(C)C)nn2c1CN(c1nc(OC[C@@]34CCCN3C[C@H](F)C4)nc3c1COC(c1c(F)c(N)cc(Cl)c1C(F)(F)F)C3)CCC2] 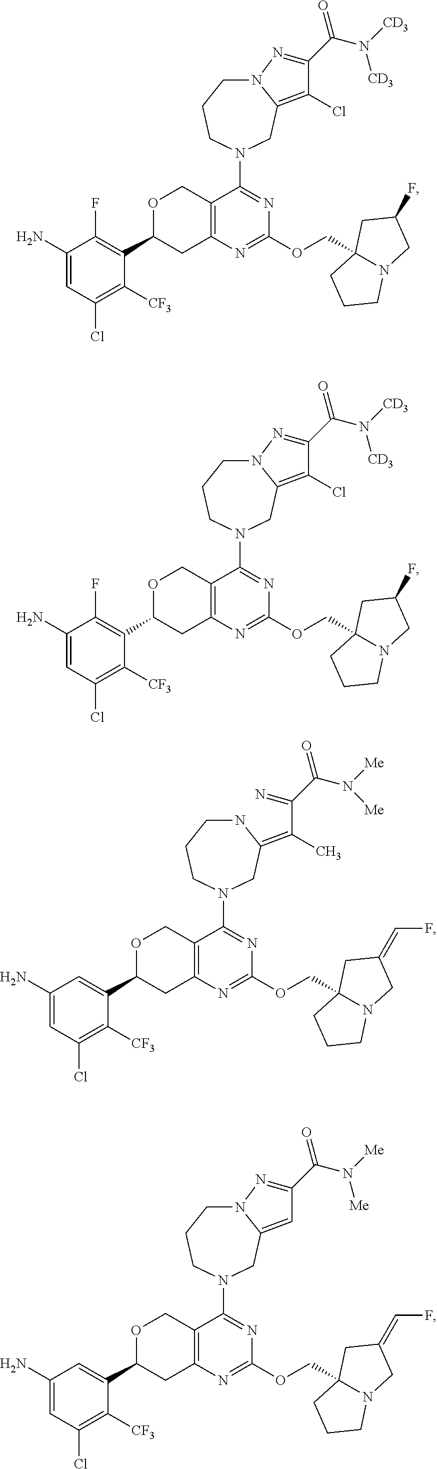 [SMILES: [2H]C([2H])([2H])N(C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2)C([2H])([2H])[2H] ; [2H]C([2H])([2H])N(C(=O)c1nn2c(c1Cl)CN(c1nc(OC[C@@]34CCCN3C[C@H](F)C4)nc3c1CO[C@@H](c1c(F)c(N)cc(Cl)c1C(F)(F)F)C3)CCC2)C([2H])([2H])[2H] ; C/C(C(=N)C(=O)N(C)C)=C1\CN(c2nc(OC[C@@]34CCCN3C/C(=C\F)C4)nc3c2CO[C@H](c2cc(N)cc(Cl)c2C(F)(F)F)C3)CCCN1 ; CN(C)C(=O)c1cc2n(n1)CCCN(c1nc(OC[C@@]34CCCN3C/C(=C\F)C4)nc3c1CO[C@H](c1cc(N)cc(Cl)c1C(F)(F)F)C3)C2] 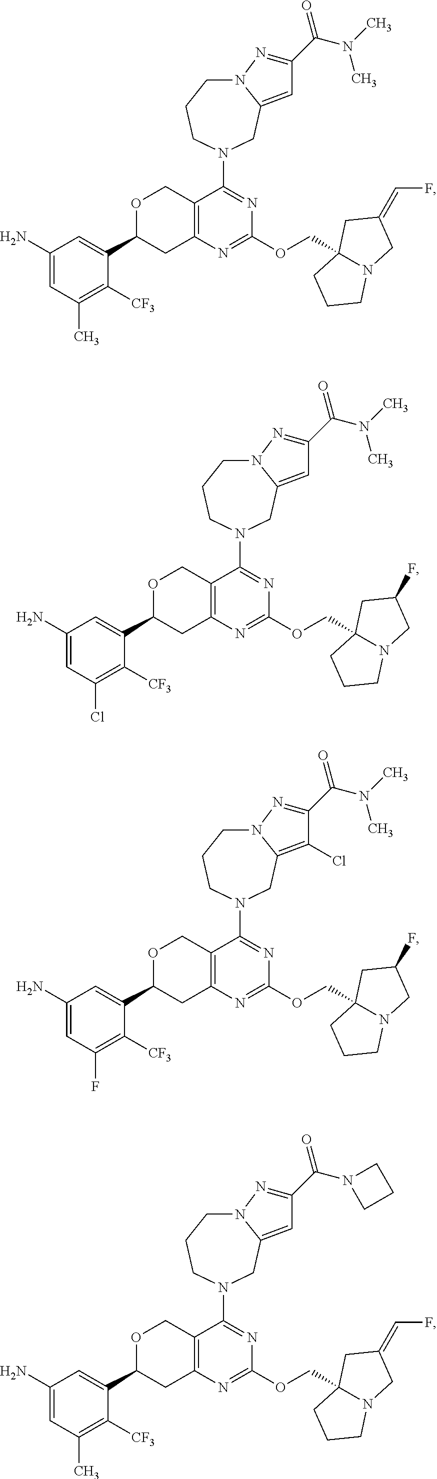 [SMILES: Cc1cc(N)cc([C@@H]2Cc3nc(OC[C@@]45CCCN4C/C(=C\F)C5)nc(N4CCCn5nc(C(=O)N(C)C)cc5C4)c3CO2)c1C(F)(F)F ; CN(C)C(=O)c1cc2n(n1)CCCN(c1nc(OC[C@@]34CCCN3C[C@H](F)C4)nc3c1CO[C@H](c1cc(N)cc(Cl)c1C(F)(F)F)C3)C2 ; CN(C)C(=O)c1nn2c(c1Cl)CN(c1nc(OC[C@@]34CCCN3C[C@H](F)C4)nc3c1CO[C@H](c1cc(N)cc(F)c1C(F)(F)F)C3)CCC2 ; Cc1cc(N)cc([C@@H]2Cc3nc(OC[C@@]45CCCN4C/C(=C\F)C5)nc(N4CCCn5nc(C(=O)N6CCC6)cc5C4)c3CO2)c1C(F)(F)F] 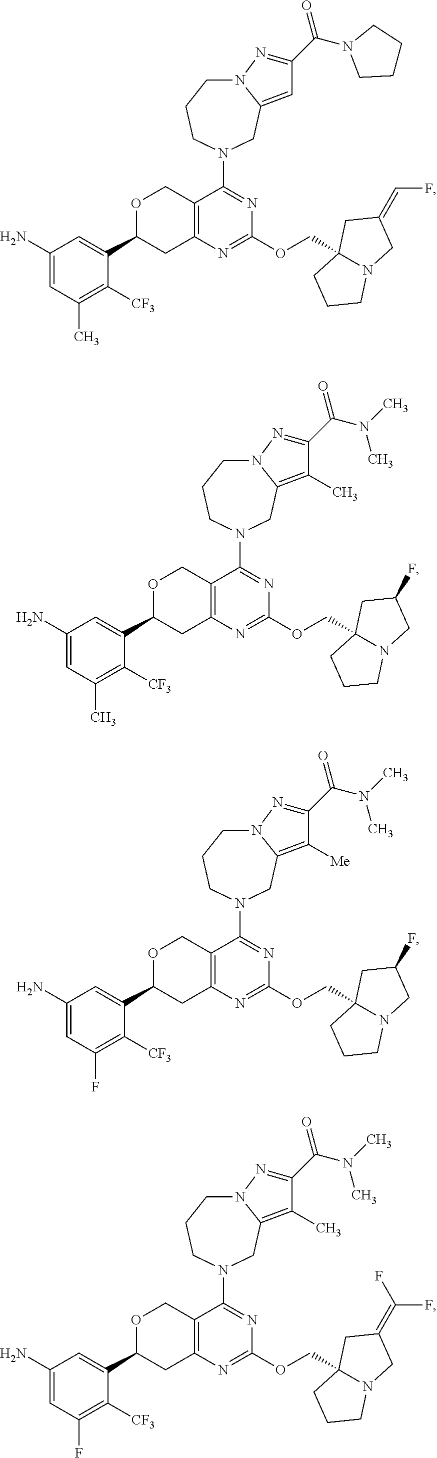 [SMILES: Cc1cc(N)cc([C@@H]2Cc3nc(OC[C@@]45CCCN4C/C(=C\F)C5)nc(N4CCCn5nc(C(=O)N6CCCC6)cc5C4)c3CO2)c1C(F)(F)F ; Cc1cc(N)cc([C@@H]2Cc3nc(OC[C@@]45CCCN4C[C@H](F)C5)nc(N4CCCn5nc(C(=O)N(C)C)c(C)c5C4)c3CO2)c1C(F)(F)F ; Cc1c(C(=O)N(C)C)nn2c1CN(c1nc(OC[C@@]34CCCN3C[C@H](F)C4)nc3c1CO[C@H](c1cc(N)cc(F)c1C(F)(F)F)C3)CCC2 ; Cc1c(C(=O)N(C)C)nn2c1CN(c1nc(OC[C@@]34CCCN3CC(=C(F)F)C4)nc3c1CO[C@H](c1cc(N)cc(F)c1C(F)(F)F)C3)CCC2] 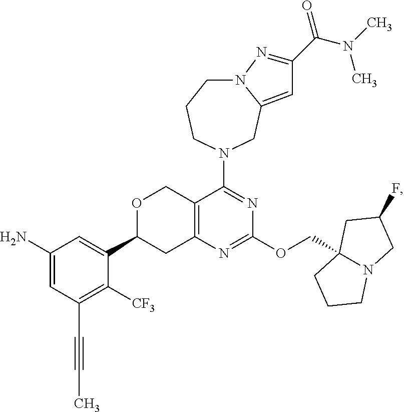 [SMILES: CC#Cc1cc(N)cc([C@@H]2Cc3nc(OC[C@@]45CCCN4C[C@H](F)C5)nc(N4CCCn5nc(C(=O)N(C)C)cc5C4)c3CO2)c1C(F)(F)F] 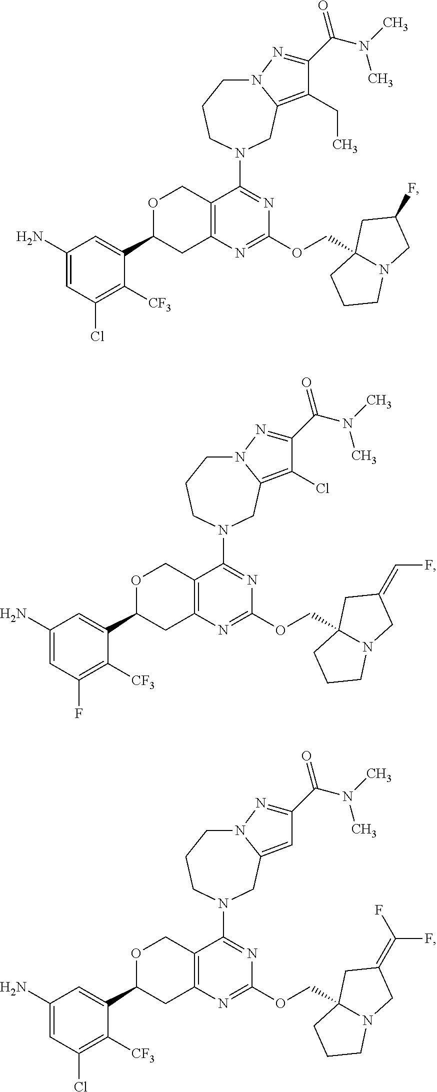 [SMILES: CCc1c(C(=O)N(C)C)nn2c1CN(c1nc(OC[C@@]34CCCN3C[C@H](F)C4)nc3c1CO[C@H](c1cc(N)cc(Cl)c1C(F)(F)F)C3)CCC2 ; CN(C)C(=O)c1nn2c(c1Cl)CN(c1nc(OC[C@@]34CCCN3C/C(=C\F)C4)nc3c1CO[C@H](c1cc(N)cc(F)c1C(F)(F)F)C3)CCC2 ; CN(C)C(=O)c1cc2n(n1)CCCN(c1nc(OC[C@@]34CCCN3CC(=C(F)F)C4)nc3c1CO[C@H](c1cc(N)cc(Cl)c1C(F)(F)F)C3)C2] 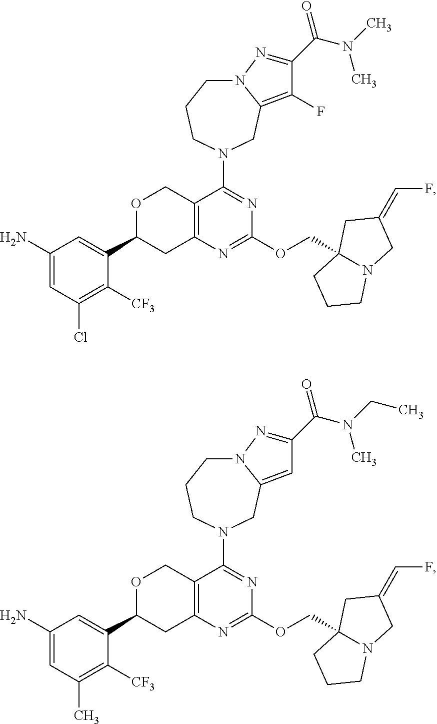 [SMILES: CN(C)C(=O)c1nn2c(c1F)CN(c1nc(OC[C@@]34CCCN3C/C(=C\F)C4)nc3c1CO[C@H](c1cc(N)cc(Cl)c1C(F)(F)F)C3)CCC2 ; CCN(C)C(=O)c1cc2n(n1)CCCN(c1nc(OC[C@@]34CCCN3C/C(=C\F)C4)nc3c1CO[C@H](c1cc(N)cc(C)c1C(F)(F)F)C3)C2] 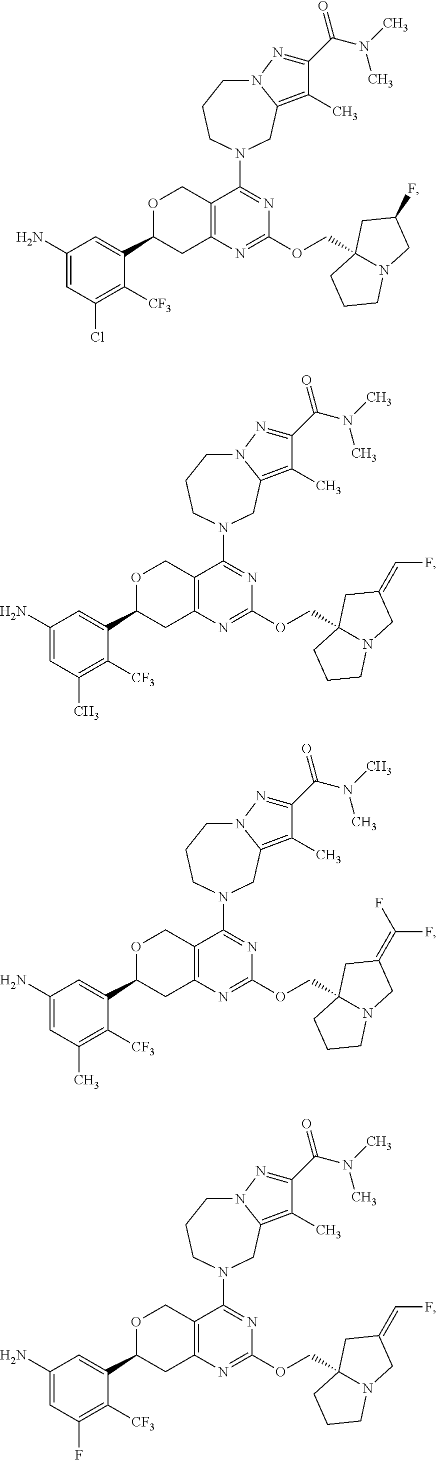 [SMILES: Cc1c(C(=O)N(C)C)nn2c1CN(c1nc(OC[C@@]34CCCN3C[C@H](F)C4)nc3c1CO[C@H](c1cc(N)cc(Cl)c1C(F)(F)F)C3)CCC2 ; Cc1cc(N)cc([C@@H]2Cc3nc(OC[C@@]45CCCN4C/C(=C\F)C5)nc(N4CCCn5nc(C(=O)N(C)C)c(C)c5C4)c3CO2)c1C(F)(F)F ; Cc1cc(N)cc([C@@H]2Cc3nc(OC[C@@]45CCCN4CC(=C(F)F)C5)nc(N4CCCn5nc(C(=O)N(C)C)c(C)c5C4)c3CO2)c1C(F)(F)F ; Cc1c(C(=O)N(C)C)nn2c1CN(c1nc(OC[C@@]34CCCN3C/C(=C\F)C4)nc3c1CO[C@H](c1cc(N)cc(F)c1C(F)(F)F)C3)CCC2] 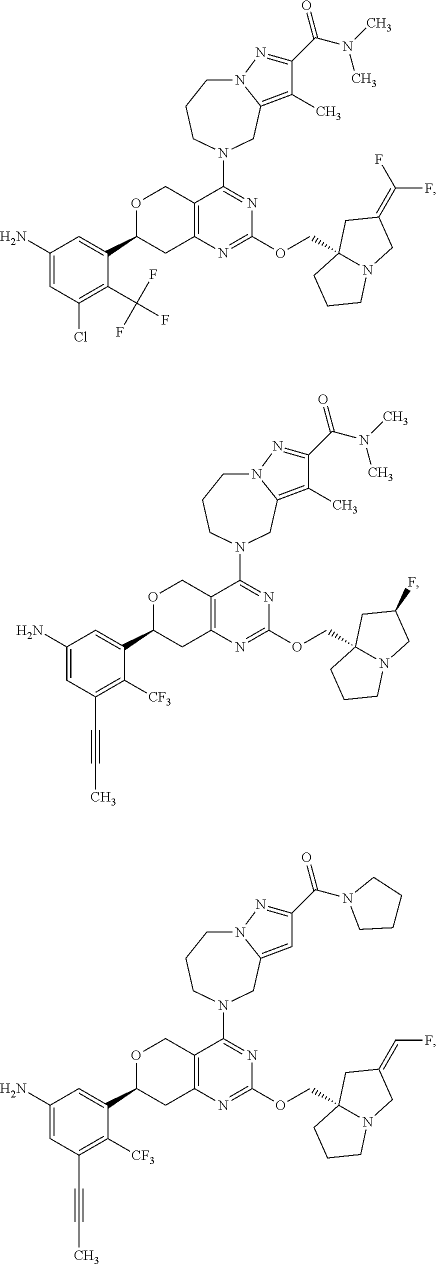 [SMILES: Cc1c(C(=O)N(C)C)nn2c1CN(c1nc(OC[C@@]34CCCN3CC(=C(F)F)C4)nc3c1CO[C@H](c1cc(N)cc(Cl)c1C(F)(F)F)C3)CCC2 ; CC#Cc1cc(N)cc([C@@H]2Cc3nc(OC[C@@]45CCCN4C[C@H](F)C5)nc(N4CCCn5nc(C(=O)N(C)C)c(C)c5C4)c3CO2)c1C(F)(F)F ; CC#Cc1cc(N)cc([C@@H]2Cc3nc(OC[C@@]45CCCN4C/C(=C\F)C5)nc(N4CCCn5nc(C(=O)N6CCCC6)cc5C4)c3CO2)c1C(F)(F)F] 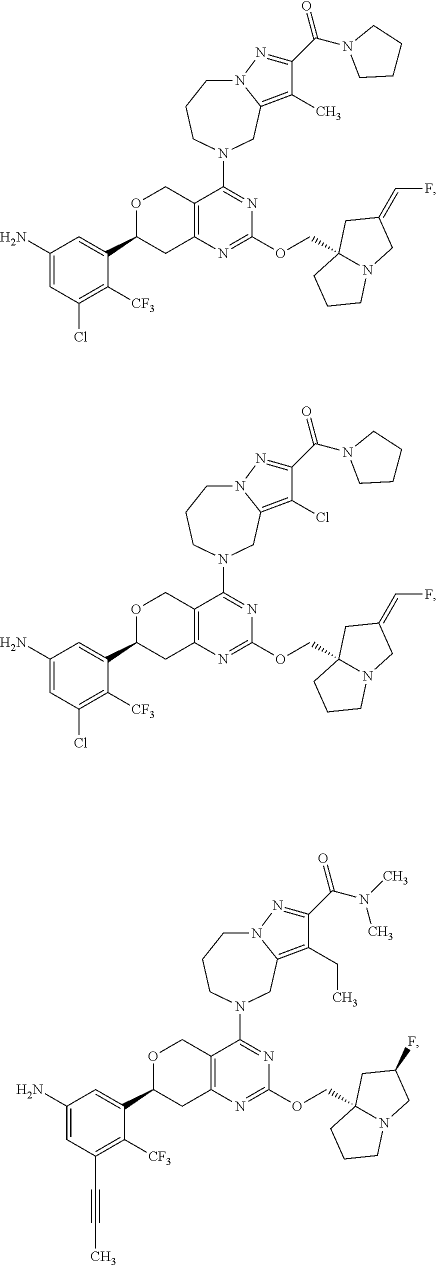 [SMILES: Cc1c(C(=O)N2CCCC2)nn2c1CN(c1nc(OC[C@@]34CCCN3C/C(=C\F)C4)nc3c1CO[C@H](c1cc(N)cc(Cl)c1C(F)(F)F)C3)CCC2 ; Nc1cc(Cl)c(C(F)(F)F)c([C@@H]2Cc3nc(OC[C@@]45CCCN4C/C(=C\F)C5)nc(N4CCCn5nc(C(=O)N6CCCC6)c(Cl)c5C4)c3CO2)c1 ; CC#Cc1cc(N)cc([C@@H]2Cc3nc(OC[C@@]45CCCN4C[C@H](F)C5)nc(N4CCCn5nc(C(=O)N(C)C)c(CC)c5C4)c3CO2)c1C(F)(F)F] 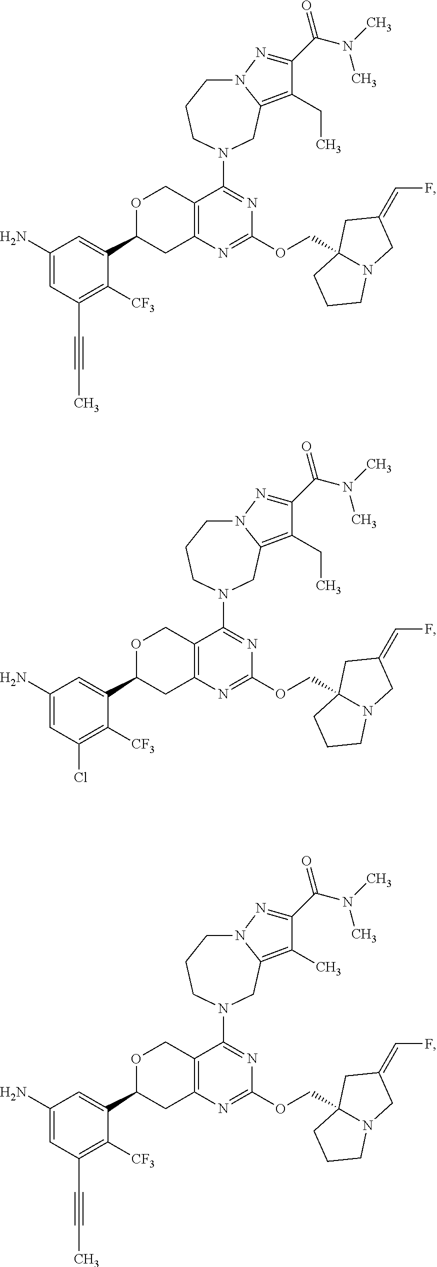 [SMILES: CC#Cc1cc(N)cc([C@@H]2Cc3nc(OC[C@@]45CCCN4C/C(=C\F)C5)nc(N4CCCn5nc(C(=O)N(C)C)c(CC)c5C4)c3CO2)c1C(F)(F)F ; CCc1c(C(=O)N(C)C)nn2c1CN(c1nc(OC[C@@]34CCCN3C/C(=C\F)C4)nc3c1CO[C@H](c1cc(N)cc(Cl)c1C(F)(F)F)C3)CCC2 ; CC#Cc1cc(N)cc([C@@H]2Cc3nc(OC[C@@]45CCCN4C/C(=C\F)C5)nc(N4CCCn5nc(C(=O)N(C)C)c(C)c5C4)c3CO2)c1C(F)(F)F] 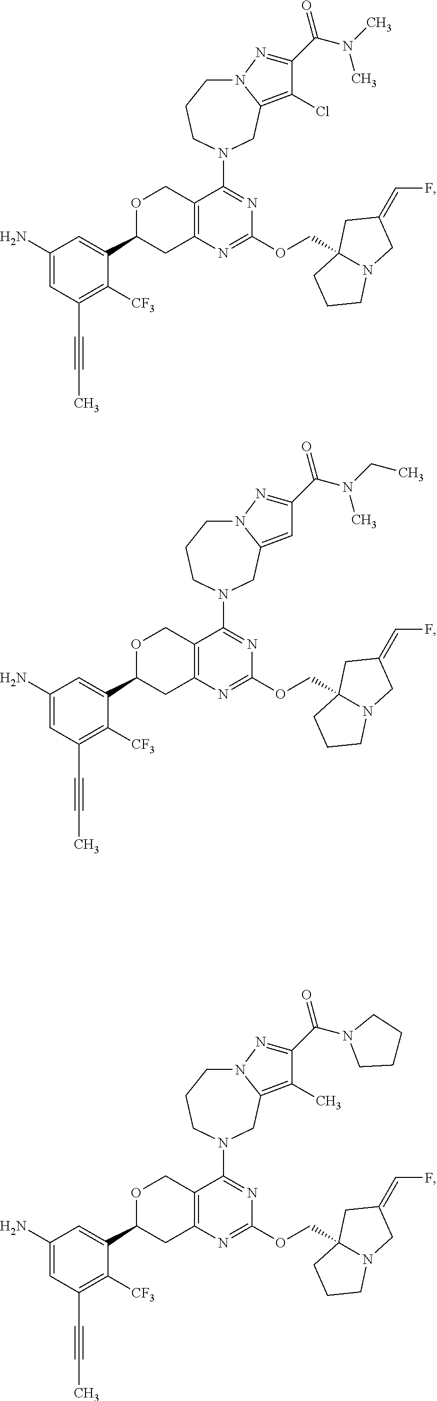 [SMILES: CC#Cc1cc(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F ; CC#Cc1cc(N)cc([C@@H]2Cc3nc(OC[C@@]45CCCN4C/C(=C\F)C5)nc(N4CCCn5nc(C(=O)N(C)CC)cc5C4)c3CO2)c1C(F)(F)F ; CC#Cc1cc(N)cc([C@@H]2Cc3nc(OC[C@@]45CCCN4C/C(=C\F)C5)nc(N4CCCn5nc(C(=O)N6CCCC6)c(C)c5C4)c3CO2)c1C(F)(F)F] 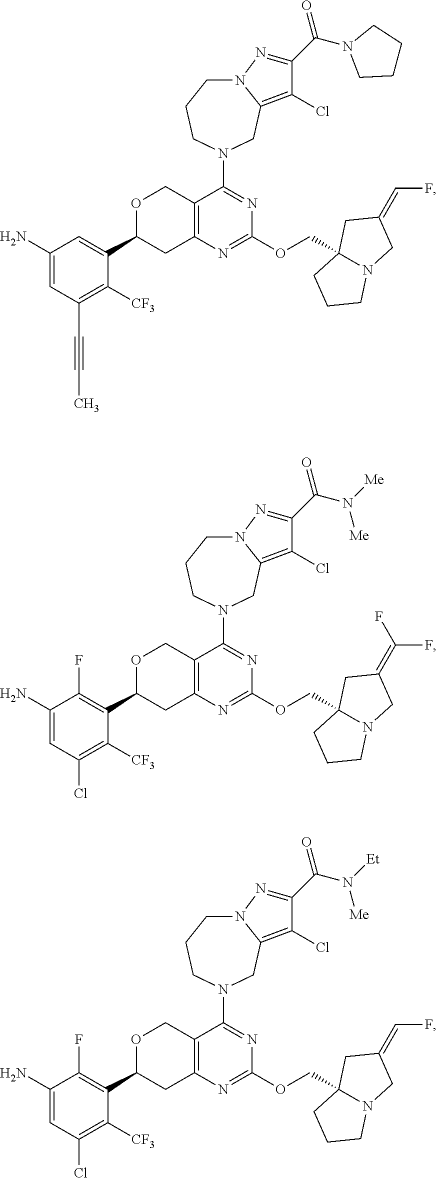 [SMILES: CC#Cc1cc(N)cc([C@@H]2Cc3nc(OC[C@@]45CCCN4C/C(=C\F)C5)nc(N4CCCn5nc(C(=O)N6CCCC6)c(Cl)c5C4)c3CO2)c1C(F)(F)F ; CN(C)C(=O)c1nn2c(c1Cl)CN(c1nc(OC[C@@]34CCCN3CC(=C(F)F)C4)nc3c1CO[C@H](c1c(F)c(N)cc(Cl)c1C(F)(F)F)C3)CCC2 ; CCN(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2] 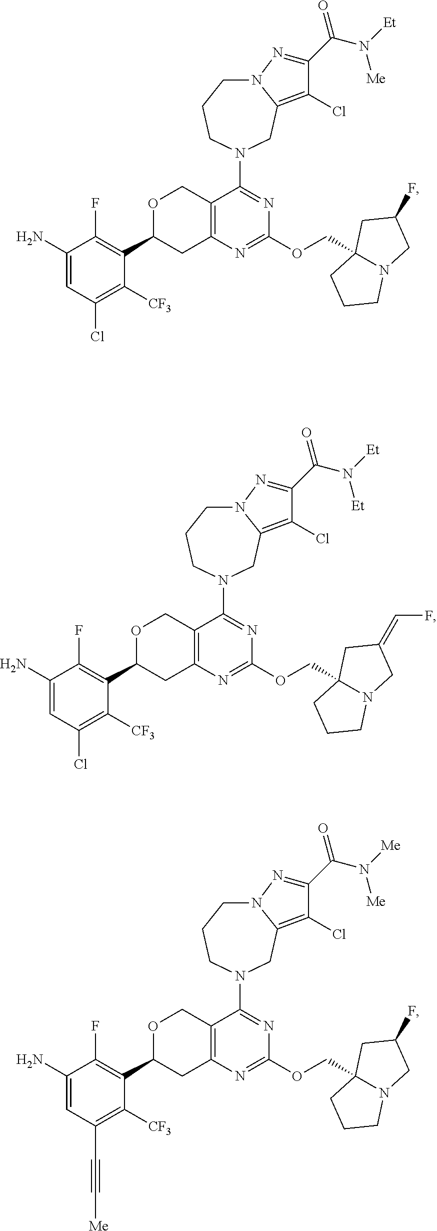 [SMILES: CCN(C)C(=O)c1nn2c(c1Cl)CN(c1nc(OC[C@@]34CCCN3C[C@H](F)C4)nc3c1CO[C@H](c1c(F)c(N)cc(Cl)c1C(F)(F)F)C3)CCC2 ; CCN(CC)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2 ; CC#Cc1cc(N)c(F)c([C@@H]2Cc3nc(OC[C@@]45CCCN4C[C@H](F)C5)nc(N4CCCn5nc(C(=O)N(C)C)c(Cl)c5C4)c3CO2)c1C(F)(F)F] 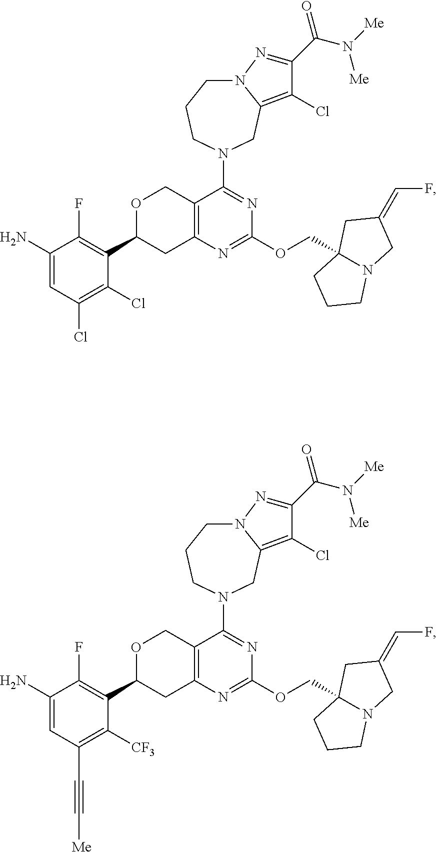 [SMILES: CN(C)C(=O)c1nn2c(c1Cl)CN(c1nc(OC[C@@]34CCCN3C/C(=C\F)C4)nc3c1CO[C@H](c1c(F)c(N)cc(Cl)c1Cl)C3)CCC2 ; CC#Cc1cc(N)c(F)c([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F] 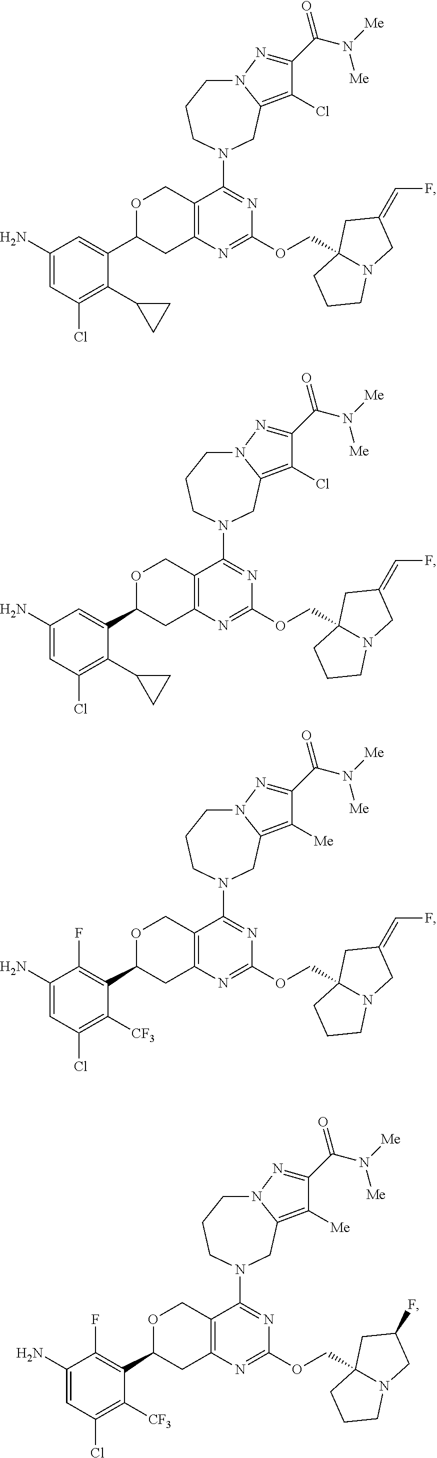 [SMILES: CN(C)C(=O)c1nn2c(c1Cl)CN(c1nc(OC[C@@]34CCCN3C/C(=C\F)C4)nc3c1COC(c1cc(N)cc(Cl)c1C1CC1)C3)CCC2 ; CN(C)C(=O)c1nn2c(c1Cl)CN(c1nc(OC[C@@]34CCCN3C/C(=C\F)C4)nc3c1CO[C@H](c1cc(N)cc(Cl)c1C1CC1)C3)CCC2 ; Cc1c(C(=O)N(C)C)nn2c1CN(c1nc(OC[C@@]34CCCN3C/C(=C\F)C4)nc3c1CO[C@H](c1c(F)c(N)cc(Cl)c1C(F)(F)F)C3)CCC2 ; Cc1c(C(=O)N(C)C)nn2c1CN(c1nc(OC[C@@]34CCCN3C[C@H](F)C4)nc3c1CO[C@H](c1c(F)c(N)cc(Cl)c1C(F)(F)F)C3)CCC2] 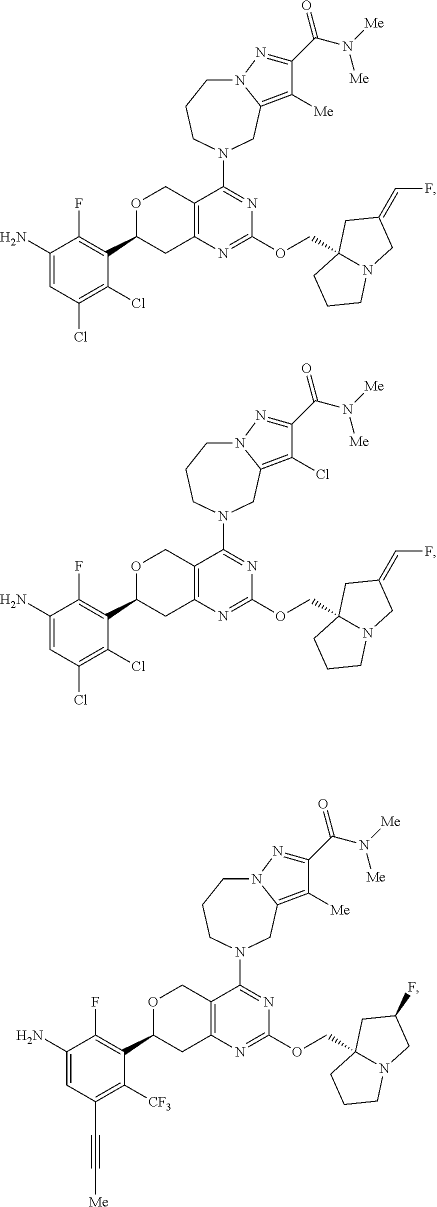 [SMILES: Cc1c(C(=O)N(C)C)nn2c1CN(c1nc(OC[C@@]34CCCN3C/C(=C\F)C4)nc3c1CO[C@H](c1c(F)c(N)cc(Cl)c1Cl)C3)CCC2 ; CN(C)C(=O)c1nn2c(c1Cl)CN(c1nc(OC[C@@]34CCCN3C/C(=C\F)C4)nc3c1CO[C@H](c1c(F)c(N)cc(Cl)c1Cl)C3)CCC2 ; CC#Cc1cc(N)c(F)c([C@@H]2Cc3nc(OC[C@@]45CCCN4C[C@H](F)C5)nc(N4CCCn5nc(C(=O)N(C)C)c(C)c5C4)c3CO2)c1C(F)(F)F] 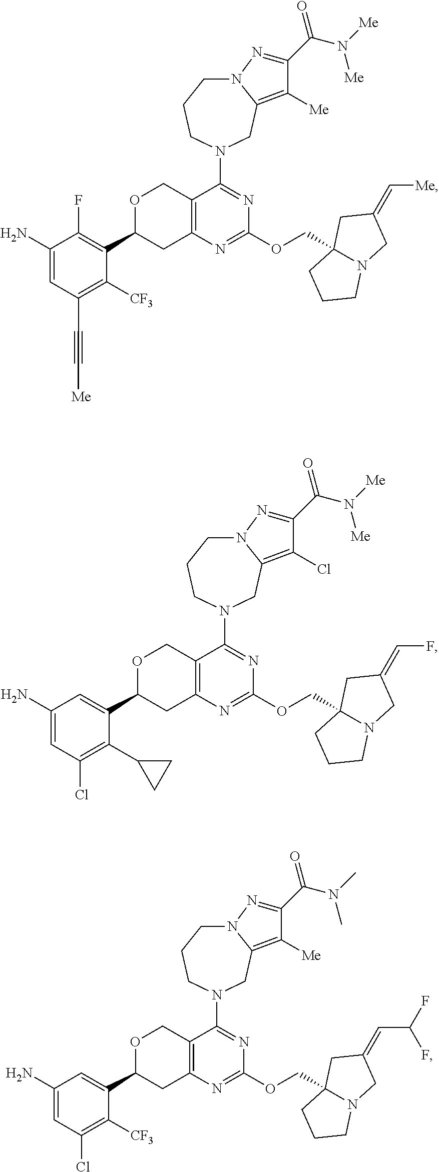 [SMILES: CC#Cc1cc(N)c(F)c([C@@H]2Cc3nc(OC[C@@]45CCCN4C/C(=C\C)C5)nc(N4CCCn5nc(C(=O)N(C)C)c(C)c5C4)c3CO2)c1C(F)(F)F ; CN(C)C(=O)c1nn2c(c1Cl)CN(c1nc(OC[C@@]34CCCN3C/C(=C\F)C4)nc3c1CO[C@H](c1cc(N)cc(Cl)c1C1CC1)C3)CCC2 ; Cc1c(C(=O)N(C)C)nn2c1CN(c1nc(OC[C@@]34CCCN3C/C(=C\C(F)F)C4)nc3c1CO[C@H](c1cc(N)cc(Cl)c1C(F)(F)F)C3)CCC2] 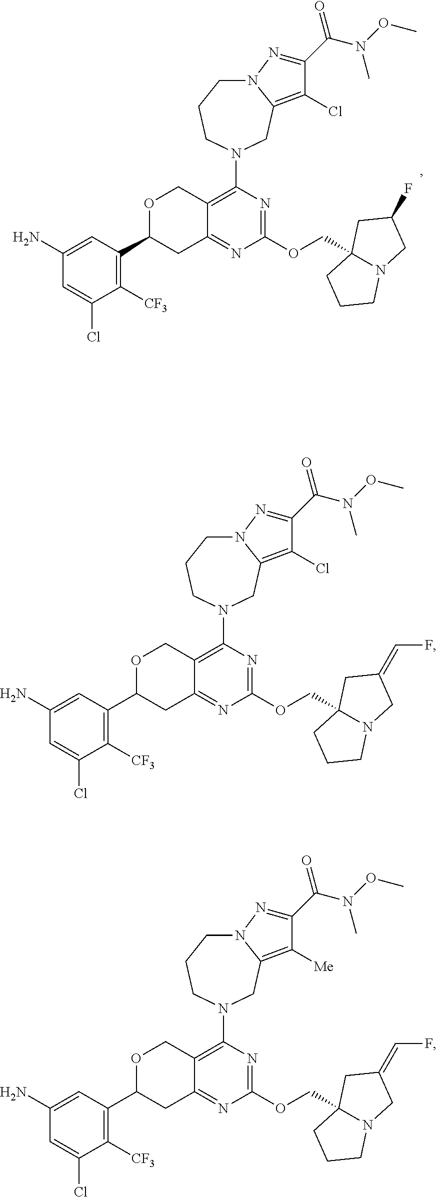 [SMILES: CON(C)C(=O)c1nn2c(c1Cl)CN(c1nc(OC[C@@]34CCCN3C[C@H](F)C4)nc3c1CO[C@H](c1cc(N)cc(Cl)c1C(F)(F)F)C3)CCC2 ; CON(C)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2 ; CON(C)C(=O)c1nn2c(c1C)CN(c1nc(OC[C@@]34CCCN3C/C(=C\F)C4)nc3c1COC(c1cc(N)cc(Cl)c1C(F)(F)F)C3)CCC2] 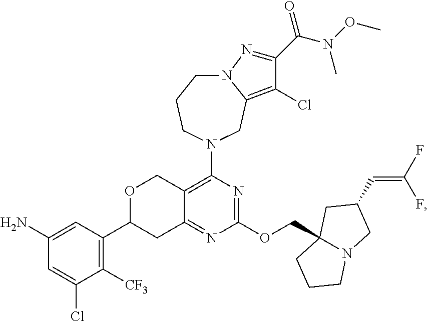 [SMILES: CON(C)C(=O)c1nn2c(c1Cl)CN(c1nc(OC[C@]34CCCN3C[C@@H](C=C(F)F)C4)nc3c1COC(c1cc(N)cc(Cl)c1C(F)(F)F)C3)CCC2] 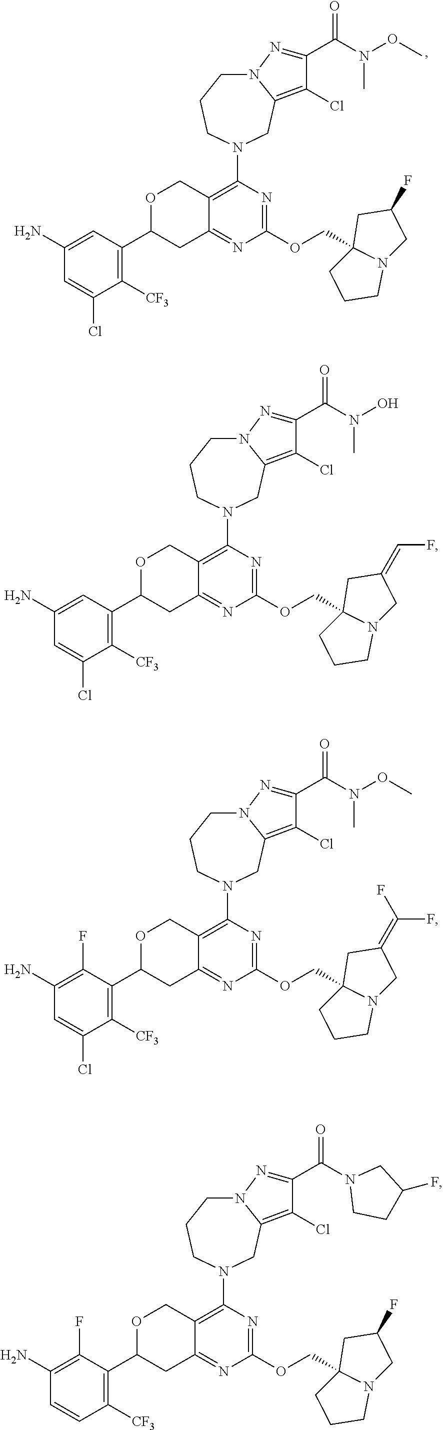 [SMILES: CON(C)C(=O)c1nn2c(c1Cl)CN(c1nc(OC[C@@]34CCCN3C[C@H](F)C4)nc3c1COC(c1cc(N)cc(Cl)c1C(F)(F)F)C3)CCC2 ; CN(O)C(=O)c1nn2c(c1Cl)CN(c1nc(OC[C@@]34CCCN3C/C(=C\F)C4)nc3c1COC(c1cc(N)cc(Cl)c1C(F)(F)F)C3)CCC2 ; CON(C)C(=O)c1nn2c(c1Cl)CN(c1nc(OC[C@@]34CCCN3CC(=C(F)F)C4)nc3c1COC(c1c(F)c(N)cc(Cl)c1C(F)(F)F)C3)CCC2 ; Nc1ccc(C(F)(F)F)c(C2Cc3nc(OC[C@@]45CCCN4C[C@H](F)C5)nc(N4CCCn5nc(C(=O)N6CCC(F)C6)c(Cl)c5C4)c3CO2)c1F] 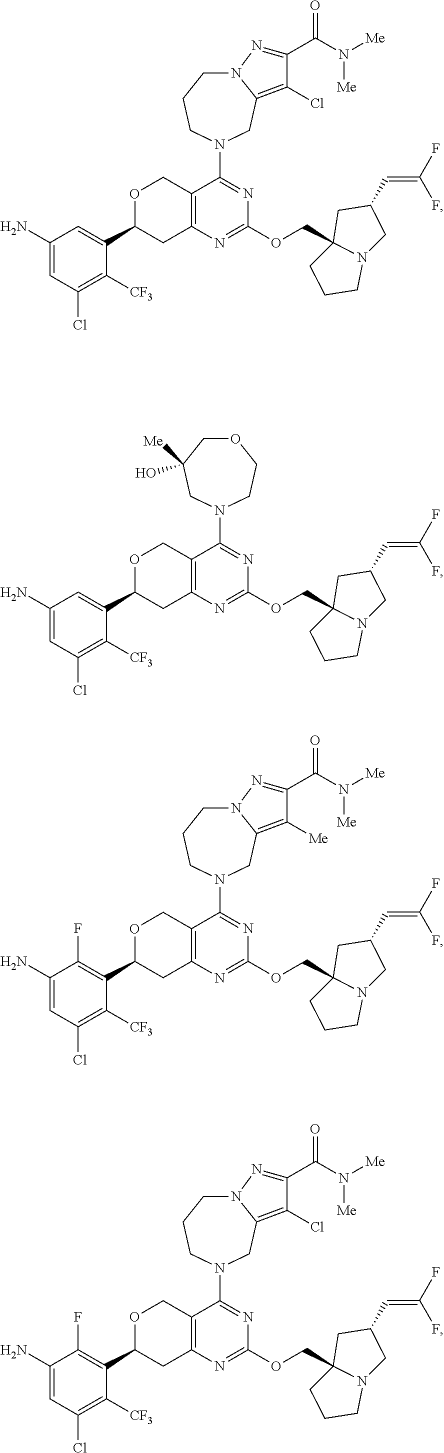 [SMILES: CN(C)C(=O)c1nn2c(c1Cl)CN(c1nc(OC[C@]34CCCN3C[C@@H](C=C(F)F)C4)nc3c1CO[C@H](c1cc(N)cc(Cl)c1C(F)(F)F)C3)CCC2 ; C[C@@]1(O)COCCN(c2nc(OC[C@]34CCCN3C[C@@H](C=C(F)F)C4)nc3c2CO[C@H](c2cc(N)cc(Cl)c2C(F)(F)F)C3)C1 ; Cc1c(C(=O)N(C)C)nn2c1CN(c1nc(OC[C@]34CCCN3C[C@@H](C=C(F)F)C4)nc3c1CO[C@H](c1c(F)c(N)cc(Cl)c1C(F)(F)F)C3)CCC2 ; CN(C)C(=O)c1nn2c(c1Cl)CN(c1nc(OC[C@]34CCCN3C[C@@H](C=C(F)F)C4)nc3c1CO[C@H](c1c(F)c(N)cc(Cl)c1C(F)(F)F)C3)CCC2] 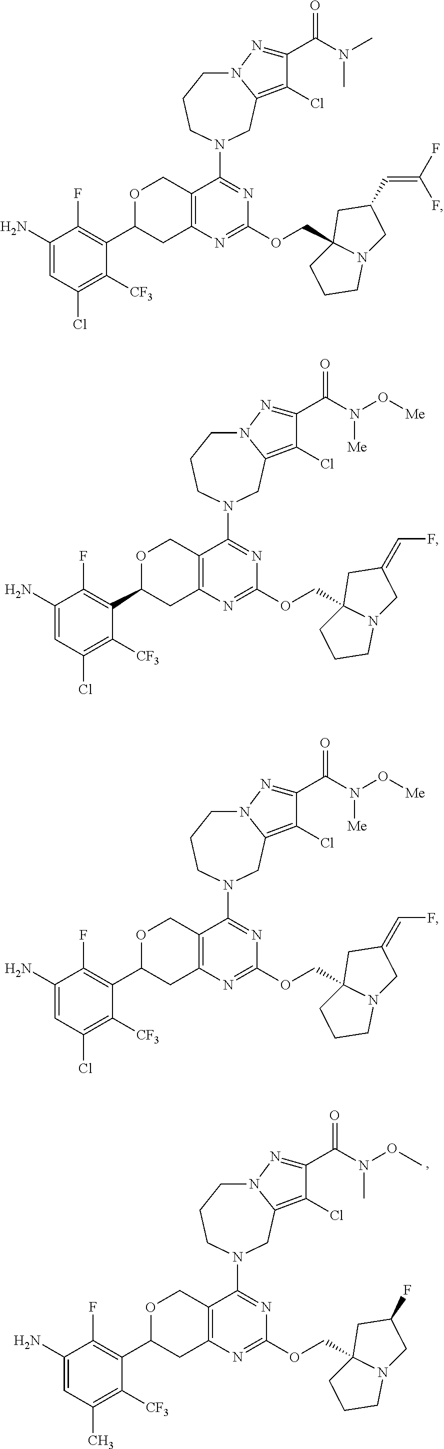 [SMILES: CN(C)C(=O)c1nn2c(c1Cl)CN(c1nc(OC[C@]34CCCN3C[C@@H](C=C(F)F)C4)nc3c1COC(c1c(F)c(N)cc(Cl)c1C(F)(F)F)C3)CCC2 ; CON(C)C(=O)c1nn2c(c1Cl)CN(c1nc(OC[C@@]34CCCN3C/C(=C\F)C4)nc3c1CO[C@H](c1c(F)c(N)cc(Cl)c1C(F)(F)F)C3)CCC2 ; CON(C)C(=O)c1nn2c(c1Cl)CN(c1nc(OC[C@@]34CCCN3C/C(=C\F)C4)nc3c1COC(c1c(F)c(N)cc(Cl)c1C(F)(F)F)C3)CCC2 ; CON(C)C(=O)c1nn2c(c1Cl)CN(c1nc(OC[C@@]34CCCN3C[C@H](F)C4)nc3c1COC(c1c(F)c(N)cc(C)c1C(F)(F)F)C3)CCC2] 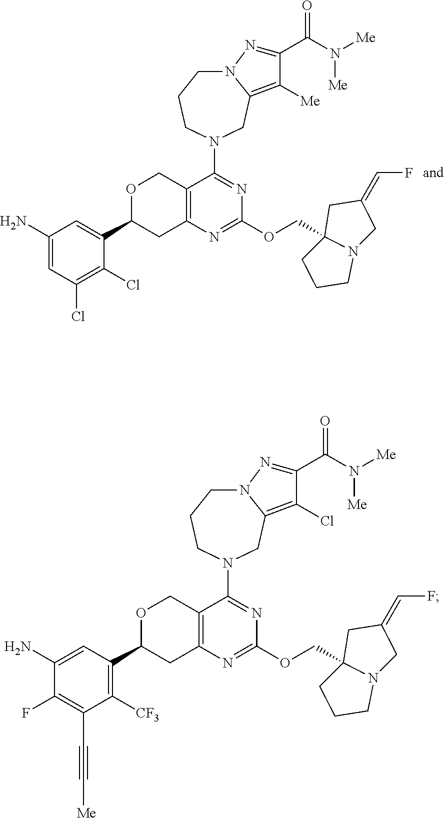 [SMILES: Cc1c(C(=O)N(C)C)nn2c1CN(c1nc(OC[C@@]34CCCN3C/C(=C\F)C4)nc3c1CO[C@H](c1cc(N)cc(Cl)c1Cl)C3)CCC2 ; CC#Cc1c(F)c(N)cc([C@@H]2Cc3nc(OC[C@@]45CCCN4C/C(=C\F)C5)nc(N4CCCn5nc(C(=O)N(C)C)c(Cl)c5C4)c3CO2)c1C(F)(F)F]
or a stereoisomer, a tautomer, or a pharmaceutically acceptable salt thereof.

15. A pharmaceutical composition comprising a pharmaceutically effective amount of a compound of any one of claims 1 to 14, or a pharmaceutically acceptable salt thereof, and a pharmaceutically acceptable excipient.

16. The pharmaceutical composition of claim 15 further comprising an additional therapeutic agent.

17. A method of treating a subject having cancer, the cancer characterized by the presence of a KRAS G12D or G12V or KRAS G12V mutation, the method comprising administering to the subject a therapeutically effective amount of a compound of any one of claims 1 to 14, or a pharmaceutically acceptable salt thereof, or a pharmaceutical composition thereof.

18. The method of claim 17, wherein the cancer is Cardiac: sarcoma, (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

19. The method of claim 17, wherein the cancer is non-small cell lung cancer, small cell lung cancer, colorectal cancer, rectal cancer, or pancreatic cancer.

20. Use of a compound of any one of claims 1 to 14, or a pharmaceutically acceptable salt thereof, or a a pharmaceutical composition thereof, in the manufacture of a medicament for the treatment of cancer in a subject, the cancer characterized by the presence of a KRAS G12D or G12V mutation.

21. The use of claim 20, wherein the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

22. The use of claim 20, wherein the cancer is non-small cell lung cancer, small cell lung cancer, colorectal cancer, rectal cancer, or pancreatic cancer.
